(19) United States
(12) Patent Application Publication
(10) Pub. No.: US 2025/0059247 A1
(43) Pub. Date: Feb. 20, 2025

(54) INTERLEUKIN 21 VARIANTS

(71) Applicant: A-Alpha Bio, Inc., Seattle, WA (US)

(72) Inventors: Davis Goodnight, Seattle, WA (US); Emily Engelhart, Seattle, WA (US); Ryan Swanson, Seattle, WA (US)

(21) Appl. No.: 18/804,738

(22) Filed: Aug. 14, 2024

Related U.S. Application Data
(60) Provisional application No. 63/519,389, filed on Aug. 14, 2023.
(60) Provisional application No. 63/574,538, filed on Apr. 4, 2024.

(51) Int. Cl. C07K 14/54

(52) U.S. Cl. CPC C07K 14/54

(57) ABSTRACT

The disclosure provides interleukin 21 (IL-21) variants that have (i) decreased or no binding to a human interleukin 21 receptor (IL-21R) as compared to a wild-type human IL-21 polypeptide; or (ii) increased stability as compared to a wild-type human IL-21 polypeptide; or (iii) decreased or no binding to a human interleukin 21 receptor (IL-21R) as compared to a wild-type human IL-21 polypeptide and increased stability as compared to a wild-type human IL-21 polypeptide. The disclosure also provides fusion proteins including an antibody or fragment thereof bound, e.g., covalently linked, to an IL-21 variant.

### CROSS-REFERENCE TO RELATED APPLICATIONS

This application claims the benefit of U.S. Provisional Patent Application No. 63/519,389, filed Aug. 14, 2023, and U.S. Provisional Patent Application No. 63/574,538, filed Apr. 4, 2024, the contents of which are incorporated herein by reference in their entirety.

### TECHNICAL FIELD

The present disclosure provides human interleukin 21 (IL-21) variants and fusion proteins including the IL-21 variants, and methods of making and using them.

### BACKGROUND

Cytokines, including IL-21, are potent immune modulators with potential therapeutic value in immuno-oncology and infectious disease. However, in human subjects their potency can present challenges, including only modest efficacies accompanied by significant toxicities and adverse side effects due to immune activation in healthy tissues. Therefore, there exists a need for therapeutic agents based on a targeted cytokine, which would be of great clinical value in treatments of various diseases including cancer.

### SUMMARY

The present disclosure provides human interleukin 21 (IL-21) variants and fusion proteins including those variants and methods for measuring binding affinities between IL-21 variants and the human interleukin 21 receptor (IL-21R). The provided IL-21 variants and fusion proteins comprising IL-21 variants can have therapeutic use. Also provided are methods of stimulating an immune response or suppressing cellular/tumor proliferation in a mammal, and methods of treating a disorder (e.g., cancer) using the IL-21 variants or the fusion proteins of such IL-21 variants.

In a therapeutic context, these cytokine variants can be targeted to a particular target cell, e.g., a type of immune cell, of interest by linking the cytokine to an antibody or portion thereof against a specific cell surface marker of the target cell. To ensure immune activation occurs primarily or only at the cell type of interest, the disclosure provides “detuned” cytokine variants, e.g., IL-21 variants, with weakened affinity for their receptors (in this case, IL-21 variants with weakened affinity for interleukin 21 receptor (IL-21R)). Detuning the cytokine can decrease or eliminate toxicities and adverse side effects by localizing the cytokine activity to specific cellular or tumor contexts. By reducing affinity for the receptor, the activity of the detuned cytokines is decreased for most cell types, e.g., in healthy tissues, which, in turn, decreases potential toxicity of the molecule when administered as a therapy. However, at the surface of the cell type targeted by the antibody, the residual activity of the cytokine is sufficient to bind its receptor and activate pro-inflammatory pathways leading to immune activation.

The compositions and methods provided herein are based, at least in part, on the identification of IL-21 variants with decreased affinity for the IL-21R receptor by high-throughput screening using a protein-protein interaction (PPI) assay based on a yeast sexual agglutination method, termed AlphaSeq™ (see, e.g., U.S. Pat. Nos. 10,988,759 and 11,136,573).

Also described herein are IL-21 variants that exhibit increased stability, either expressed alone or in the context of a fusion protein. Given the biophysical liabilities of IL-21, stabilization of an IL-21 therapeutic is desirable. Stabilization can include various parameters and be measured in by various methods known in the art. Increased stability can include higher expression titers in cells, e.g., CHO cells, during manufacturing; increased monomeric species of interest measured by analytical size exclusion chromatography (SEC) following purification; or higher melting temperature in differential scanning fluorimetry (DSF). Additionally, stabilized can have longer half-lives in serum stability assays in vitro or enhanced pharmacokinetic properties when administered to an animal or patient as a drug.

Cytokine signaling can trigger multifaceted and even opposing activities in different cell types, often leading to toxicity or poor response rates in the therapeutic setting. Targeting specific cellular signaling, such as defined immune subsets or antigen experienced cells at the tumor, has the potential to widen a cytokine therapeutic index and improve patient outcomes.

In a first aspect, the disclosure provides human interleukin 21 (IL-21) variants, wherein the IL-21 variants each have (i) decreased or no binding to a human interleukin 21 receptor (IL-21R) as compared to a wild-type human IL-21 polypeptide; or (ii) increased stability as compared to a wild-type human IL-21 polypeptide; or (iii) decreased or no binding to a human interleukin 21 receptor (IL-21R) as compared to a wild-type human IL-21 polypeptide and increased stability as compared to a wild-type human IL-21 polypeptide. In some embodiments, the IL-21 variant has a binding affinity to the human IL-21R that is decreased by 1.5-fold, 2-fold, 3-fold, 5-fold, 10-fold, 50-fold, 100-fold, 500-fold, 1000-fold, 2000-fold, or 3000-fold, or more, relative to the binding affinity between the wild-type human IL-21 polypeptide and the human IL-21R.

In some embodiments, the IL-21 variant includes one or more amino acid substitutions at one or more positions of SEQ ID NO: 1 selected from the group consisting of: Q32, D33, R34, H35, M36, I37, R38, M39, Q41, L42, I43, D44, I45, V46, D47, Q48, L49, N51, Y52, V53, N54, D55, L56, V57, P58, E59, F60, L61, P62, A63, E65, D66, V67, E68, N70, E72, W73, 574, A75, F76, 577, F79, Q80, K81, A82, Q83, L84, K85, S86, A87, N88, T89, G90, N92, E93, R94, I95, I96, N97, V98, S99, K101, I100, K102, L103, K104, R105, K106, P107, P108, S109, T110, N111, A112, G113, R114, R115, H118, L120, K131, P132, P133, R139, S142, L144, Q145, I148, Q150, H151, and L152.

In some embodiments, the IL-21 variant includes one or more amino acid substitutions selected from the group consisting of: Q32K, Q32I, D33H, D33A, D33N, D33P, D33Q, D33R, D33S, D33T, D33W, D33V, R34A, R34D, R34E, R34F, R34G, R34H, R34K, R34L, R34M, R34N, R34P, R34Q, R34S, R34T, R34V, R34W, R34Y, H35P, H35I, H35F, M36T, M36R, M36P, I37E, I37G, I37K, I37N, I37Q, I37R, I37S, I37V, I37P, I37A, I37W, I37H, I37D, I37T, I37F, R38A, R38P, R38E, R38F, R38K, R38M, R38Q, R38V, R38W, R38N, R38G, R38L, R38S, R38T, R38I, R38D, M39D, M39E, M39G, M39I, M39K, M39P, M39Q, M39R, M39A, Q41K, Q41P, Q41S, Q41E, Q41D, Q41W, Q41R, L42D, L42E, L42G, L42H, L42K, L42N, L42Q, L42R, L42S, L42Y, I43D, I43L, I43M, I43P, I43S, I43Y, I43F, I43R, D44A, D44G, D44N, D44P, D44T, D44W, D44Y, D44F, D44Q, D44S, D44V, D44M, D44L, D44P, D44H, D44R, D44K, I45A, I45D, I45G, I45H, I45N, I45P, I45R, I45S, I45Y, I45T, I45Q, I45F, I45W, I45K, V46D, V46E, V46G, V46K, V46N, V46P, V46Q, V46T, D47P, D47S, D47R, D47K, D47F, D47G, Q48G, Q48K, Q48R, L49D, L49E, L49H, L49K, L49N, L49P, L49R, L49S, L49F, N51E, N51G, N51P, N51V, Y52D, Y52H, Y52K, Y52N, Y52Q, Y52S, Y52T, Y52R, Y52G, V53A, V53E, V53H, V53N, V53P, V53Q, V53S, V53T, V53M, N54H, N54K, N54P, D55K, D55R, D55Y, D55M, L56E, L56F, L56M, L56N, L56S, L56T, L56V, L56D, V57H, V57L, V57M, V57P, V57R, V57T, V57Y, P58M, P58K, E59R, E59S, E59V, E59Y, F60H, F60I, F60R, L61H, L61I, L61K, P62H, P62S, P62T, P62V, P62L, A63T, E65R, D66M, D66R, D66K, V67L, V67K, V67G, E68K, E68R, E68G, N70A, N70H, N70G, E72H, E72I, E72L, E72S, E72T, E72R, E72W, W73A, W73G, W73K, W73L, W73N, W73P, W73Q, W73S, W73T, W73V, W73Y, S74A, S74H, S74N, S74R, S74K, A75N, A75Y, A75V, F76D, F76E, F76H, F76M, F76P, F76Q, F76R, F76T, F76S, F76L, F76V, F76I, F76K, S77K, S77M, S77R, S77W, F79D, F79E, F79K, F79P, F79R, F79T, F79Y, F79I, F79A, F79L, F79V, Q80A, Q80D, Q80L, Q80M, Q80V, Q80I, Q80E, Q80N, Q80H, Q80W, Q80F, Q80K, Q80R, Q80G, Q80Y, K81D, K81H, A82V, A82W, A82M, A82K, A82L, A82I, A82H, A82T, A82Y, A82V, Q83L, Q83W, Q83F, Q83K, Q83H, Q83V, L84P, L84F, L84T, K85S, K85P, S86I, S86M, S86L, S86R, A87N, A87P, N88K, N88R, T89L, T89F, G90Y, G90Q, G90I, G90K, N92G, N92Y, N92W, N92P, E93G, E93T, E93R, E93F, E93P, E93K, E93S, E93V, E93I, E93Y, R94S, R94T, I95H, I95K, I95P, I95T, I95R, I95G, I96A, I96D, I96G, I96P, I96S, I96T, I96V, I96F, I96Y, I96E, I96Q, I96M, I96H, I96N, I96R, I96K, N97G, N97P, N97H, N97W, N97K, N97R, V98H, V98K, V98R, V98E, V98A, V98G, V98Y, V98N, V98S, V98M, V98T, V98P, V98F, S99H, S99R, S99P, S99K, S99G, I100E, I100R, I100S, HOOD, I100G, I100F, I100Y, I100Q, I100H, I100A, I100R, K101E, K101D, K102P, K102G, K102L, K102V, K102E, K102I, K102T, K102D, L103E, L103A, L103S, L103T, L103F, L103I, L103W, L103Y, L103V, L103Q, L103H, L103N, K104G, K104P, R105A, R105D, R105K, R105Q, R105S, R105W, R105Y, R105L, R105H, R105M, R105F, R105N, R105I, R105V, R105T, R105E, R105G, R105P, K106E, K106N, K106P, K106S, K106T, K106G, K106H, K106D, P107D, P107Y, P107V, P107H, P107V, P107K, P107R, P108D, P108E, P108G, P108I, P108L, P108Q, P108T, P108A, P108H, P108K, P108Y, P108M, P108N, P108W, P108R, S109K, S109M, S109R, T110D, T110S, T110H, T110N, N111R, N111K, A112P, A112R, A112K, G113R, G113H, R114F, R114Q, R115I, R115P, R115Q, R115D, H118I, H118K, H118R, L120H, L120K, L120M, L120V, L120Y, L120S, K131M, K131V, P132A, P133I, R139S, S142D, S142H, S142I, L144M, Q145H, I148S, Q150D, Q150R, Q150S, H151E, and L152H.

In some embodiments, the IL-21 variant is has at least 90%, 95%, 98%, 99%, or 100% sequence identity to any one of SEQ ID NOs: 462, 463, 1192, 950, 933, and 894. In some embodiments, the IL-21 variant includes one or more substitutions selected from the group consisting of R105W, R105L, R38N, R38L, R105Y, and R38W. In some embodiments, the IL-21 variant includes a polypeptide sequence selected from one of SEQ ID NOs: 15-3117.

In another aspect, the disclosure provides fusion proteins including an antibody or binding fragment thereof and a human IL-21 variant, wherein the IL-21 variant is covalently linked to the antibody or binding fragment thereof.

In another aspect, the disclosure provides a fusion protein including an antibody or binding fragment thereof including an Fc domain and a human IL-21 variant, wherein the IL-21 variant is covalently linked to the Fc domain of the antibody or binding fragment thereof.

In some embodiments, the antibody or binding fragment thereof includes an amino acid sequence 80%, 90%, 95%, 96%, 97%, 98%, 99%, or 100% identical to an amino acid sequence selected from the group consisting of SEQ ID NO: 5, 6, 7, 8, 9, 10, 11, 12, 13, or 14. In some embodiments, the antibody or binding fragment thereof includes a human antibody Fc domain including an effectorless mutations selected from NG, DANG, LALA, and LALA-PG. In some embodiments, the antibody binds to an antigen selected from the group consisting of CD8, TIGIT, PD-1, CD160, BTLA, CD25, ICOS and TNFRSF9.

In some embodiments, the antibody is selected from the group consisting of an anti-CTLA-4 antibody, an anti-CD3 antibody, an anti-CD4 antibody, an anti-CD8 antibody, an anti-4-1 BB antibody, an anti-PD-I antibody, an anti-PD-LI antibody, an anti-TlM3 antibody, an anti-LAG3 antibody, an anti-TlGIT antibody, an anti-OX40 antibody, an anti-lL-7Ralpha (CD127) antibody-, an anti-HVEM antibody, an anti-BTLA antibody, an anti-CD40 antibody, an anti-CD40L antibody, anti-CD47 antibody, an anti-CSF1R antibody, an anti-MARCO antibody, an anti-CXCR4 antibodies, an anti-VEGFRl antibody, an anti-VEGFR2 antibody, an anti-TNFRl antibody, an anti-TNFR2 antibody, an anti-CD3 bispecific antibody, an anti-CD19 antibody, an anti-CD20, an anti-Her2 antibody, an anti-EGFR antibody, an anti-lCOS antibody, an anti-CD22 antibody, an anti-CD52 antibody, an anti-CCR4 antibody, an anti-CCR5 antibody, an anti-CD200R antibody, an anti-VlSG4 antibody, an anti-CCR2 antibody, an anti-LILRB2 antibody, an anti-CXCR4 antibody, an anti-CD206 antibody, an anti-CD163 antibody, an anti-KLRGl antibody, an anti-FLT3 antibody, an anti-B7-H4 antibody, an anti-B7-H3 antibody, an KLRGl antibody, and an anti-GITR antibody, anti-CD160 antibody, anti-KLRD1 antibody, anti-KLRC1 antibody, anti-BTLA antibody, anti-LILRB4 antibody.

In some embodiments, the IL-21 variant is covalently linked to the antibody by a polypeptide linker. In some embodiments, the polypeptide linker is selected from the group consisting of (G4S)2 and (G4S)3. In some embodiments, the polypeptide linker is a polypeptide tag selected from the group consisting of FLAG, MYC, HA, and 6×his.

In another aspect, the disclosure provides cell lines that produce IL-21 variants or fusion proteins including IL-21 variants. In some embodiments, the cell line is a CHO cell line or HEK293 cell line.

In another aspect, the disclosure provides nucleic acids encoding IL-21 variants or fusion proteins including IL-21 variants. In another aspect, the disclosure provides recombinant expression vectors including nucleic acids encoding IL-21 variants or fusion proteins including IL-21 variants. In another aspect, the disclosure provides host cells including either nucleic acids encoding IL-21 variants or fusion proteins including IL-21 variants or recombinant expression vectors including nucleic acids encoding IL-21 variants or fusion proteins including IL-21 variants.

In another aspect, the disclosure provides pharmaceutical compositions including IL-21 variants fusion protein including IL-21 variants, and a pharmaceutically acceptable carrier.

In another aspect, the disclosure provides methods of treating a disease in a subject in need thereof, the method including administering to the subject the pharmaceutical composition in an amount effective to reduce one or more symptoms associated with the disease in the subject. In some embodiments, the disease is cancer. In some embodiments, the cancer is a solid cancer or a liquid cancer.

In some embodiments, the solid cancer is selected from the group consisting of gastric cancer, small intestine cancer, sarcoma, head and neck cancer, thymic cancer, epithelial cancer, salivary cancer, liver cancer, biliary cancer, neuroendocrine tumors, stomach cancer, thyroid cancer, lung cancer, mesothelioma, ovarian cancer, breast cancer, prostate cancer, esophageal cancer, pancreatic cancer, glioma, renal cancer, bladder cancer, cervical cancer, uterine cancer, vulvar cancer, penile cancer, testicular cancer, anal cancer, choriocarcinoma, colorectal cancer, oral cancer, skin cancer, Merkel cell carcinoma, glioblastoma, brain tumor, bone cancer, eye cancer, and melanoma.

In some embodiments, the liquid cancer is selected from the group consisting of multiple myeloma, malignant plasma cell neoplasm, Hodgkin's lymphoma, nodular lymphocyte predominant Hodgkin's lymphoma, Kahler's disease and Myelomatosis, plasma cell leukemia, plasmacytoma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, follicular lymphoma, marginal zone lymphoma, mucosa-associated lymphatic tissue lymphoma, small cell lymphocytic lymphoma, mantle cell lymphoma, Burkitt lymphoma, primary mediastinal (thymic) large B-cell lymphoma, lymphoplasmactyic lymphoma, Waldenstrom macroglobulinemia, nodal marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBY positive diffuse large B-cell lymphoma of the elderly, diffuse large B-cell lymphoma associated with inflammation, intravascular large B-cell lymphoma, ALKpositive large B-cell lymphoma, plasmablastic lymphoma, large B-cell lymphoma arising in HHVS-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other hematopoietic cells related cancer. In some embodiments, the cancer is relapsed, refractory, or metastatic.

In some embodiments, the method further includes administering an effective amount of a second therapeutic agent, optionally wherein the administration is separate, sequential, or simultaneous. In some embodiments, the second therapeutic agent is an antibody selected from the group consisting of an anti-CTLA-4 antibody, an anti-CD3 antibody, an anti-CD4 antibody, an anti-CD8 antibody, an anti-4-1BB antibody, an anti-PD-1 antibody, an anti-PD-Ll antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSF1R antibody, an anti-CSFl antibody, an anti-IL-7R antibody, an anti-MARCO antibody, an antiCXCR4 antibodies, an anti-VEGF antibody, an anti-VEGFRl antibody, an anti-VEGFR2 antibody, an anti-TNFRl antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD 52 antibody, an anti-CCR4 antibody, an anti-CCRS antibody, an antiCD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRGl antibody, an anti-FLT3 antibody, an anti-B7-H4 antibody, an anti-B7-H3 antibody, an KLRGl antibody, a BTNlAl antibody, and an anti-GITR antibody.

In some embodiments, the second therapeutic agent is a cytokine, an immunocytokine, TNFa, a PAP inhibitor, an oncolytic virus, a kinase inhibitor, an ALK inhibitor, a MEK inhibitor, an IDO inhibitor, a GLSl inhibitor, a tyrosine kinase inhibitor, a CART cell or T cell therapy, a TLR agonist, or a tumor vaccine.

In another aspect, the disclosure provides pharmaceutical compositions for use in the treatment of cancer, optionally wherein the cancer is a solid cancer or a liquid cancer and/or the cancer is relapsed, refractory, or metastatic. In some embodiments, the use is in combination with a second therapeutic agent, optionally wherein the combination is for administration simultaneously, concurrently, or simultaneously.

As used herein, two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison as disclosed herein is conducted using the MegAlign® program in the Lasergene® suite of bioinformatics software (DNASTAR®, Inc., Madison, Wis.), using default parameters. This program embodies several alignment schemes described in the following references: Dayhoff, M. 0., 1978, A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes, pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

As defined herein, a “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

As used herein, “protein-protein interaction” or “PPI” refers to physical contacts of high specificity established between two or more proteins (or polypeptides) as a result of biochemical events driven by electrostatic forces including the hydrophobic effect. Many are physical contacts with molecular associations between chains that occur in a cell or in a living organism in a specific biomolecular context. In some embodiments, the protein-protein interactions are strong enough to replace the function of the native sexual agglutination proteins. For example, it can be possible to couple mating efficiency to the interaction strength of a particular protein-protein interaction. In certain embodiments, the assay can characterize or determine protein-protein interactions between synthetic adhesion proteins (SAPs).

As used herein, a “synthetic adhesion protein” refers to any protein or polypeptide to be assayed for binding to or interacting with any other protein or polypeptide. The proteins can be heterologous or exogenously expressed. Synthetic adhesion proteins are referred to as such because they are not typically associated with the adhesion required for agglutination as natively performed by the sexual agglutination proteins. In certain embodiments, the synthetic adhesion proteins have sufficiently strong interactions to allow agglutination in yeast where the native sexual agglutination proteins are not natively expressed. In some embodiments, the SAPs of the first and second expression cassettes of the first and second nucleic acid constructs, respectively, bind to a cell wall GPI anchored protein. In some embodiments, the SAPs can be fused to a cell wall GPI anchored protein or fused to a protein that forms a disulfide bond with a cell wall GPI anchored protein. In some embodiments, the SAP of the first expression cassette of the first nucleic acid construct is fused to the sexual agglutination protein Aga2, and the SAP of the first expression cassette of the second nucleic acid construct is fused to the sexual agglutination protein Aga2.

As used herein, “affinity” is a measure of the strength of the binding interaction between a single biomolecule to its ligand or binding partner. Affinity is usually measured and described using the equilibrium dissociation constant, K_{d}. The lower the K_{d} value, the greater the affinity between the protein and its binding partner. Affinity may be affected by hydrogen bonding, electrostatic interactions, hydrophobic and Van der Waals forces between the binding partners, or by the presence of other molecules, e.g., binding agonists or antagonists.

In some implementations, affinity may be described using arbitrary units, wherein a certain binding affinity within an assay, for example, the binding affinity between two wildtype protein binding partners or the wild-type species of a first protein binding partner and the wild-type species of a second protein binding partner, is set to an arbitrary unit of 1.0 and binding affinities for other pairs of protein binding partners, for example the mutant species of a first protein binding partner and the mutant species of a second protein binding partner, are measured relative proportionally to that certain binding affinity.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is a heatmap of AlphaSeq™ predicted affinities (log 10 K_{d} nM) for the IL-21/IL-21R validation network in standard media conditions. Strong binding is considered below 3, weak binding is considered between 3 and 4, no binding is considered above 4.

FIG. 1B is a heatmap of AlphaSeq™ predicted affinities (log 10 K_{d} nM) for the IL-21/IL-21R validation network in pH 7 buffered conditions. Strong binding is considered below 2, weak binding is considered between 2 and 3.5, no binding is considered above 3.5.

FIG. 2A is a histogram of expression values for wild-type replicates of human IL-21. A value of 3 is the maximum possible expression in this assay and a value of 1 is the minimum possible expression.

FIG. 2B is a histogram of expression values for site-saturation mutagenesis (SSM) variants of human IL-21. A value of 3 is the maximum possible expression in this assay and a value of 1 is the minimum possible expression.

FIG. 3A is a histogram of expression values for wild-type replicates of mouse IL-21. A value of 3 is the maximum possible expression in this assay and a value of 1 is the minimum possible expression.

FIG. 3B is a histogram of expression values for site-saturation mutagenesis (SSM) variants of mouse IL-21. A value of 3 is the maximum possible expression in this assay and a value of 1 is the minimum possible expression.

FIG. 4A is a histogram of AlphaSeq™ affinity values for the replicates of wild-type human IL-21 binding to human IL-21R in unbuffered media.

FIG. 4B is a histogram of AlphaSeq™ affinity values for the replicates of wild-type human IL-21 binding to human IL-21R in pH 5 media.

FIG. 4C is a histogram of AlphaSeq™ affinity values for the replicates of wild-type human IL-21 binding to human IL-21R in pH 6 media.

FIG. 4D is a histogram of AlphaSeq™ affinity values for the replicates of wild-type human IL-21 binding to human IL-21R in pH 7 media.

FIG. 5A is a histogram of AlphaSeq™ affinity values for the full human IL-21 SSM binding to human IL-21R in unbuffered media.

FIG. 5B is a histogram of AlphaSeq™ affinity values for the full human IL-21 SSM binding to human IL-21R in pH 5 media.

FIG. 5C is a histogram of AlphaSeq™ affinity values for the full human IL-21 SSM binding to human IL-21R in pH 6 media.

FIG. 5D is a histogram of AlphaSeq™ affinity values for the full human IL-21 SSM binding to human IL-21R in pH 7 media.

FIG. 6A is a scatter plot showing the relationship between AlphaSeq™ affinity to human IL-21R (x-axis) and AlphaSeq™ affinity to cynomolgus monkey IL-21R (y-axis) for each variant in the IL-21 SSM library, measured in unbuffered media.

FIG. 6B is a scatter plot showing the relationship between AlphaSeq™ affinity to human IL-21R (x-axis) and AlphaSeq™ affinity to cynomolgus monkey IL-21R (y-axis) for each variant in the IL-21 SSM library, measured in pH 7 media.

FIG. 7A is a histogram of AlphaSeq™ affinity values for the replicates of wild-type mouse IL-21 binding to mouse IL-21R in unbuffered media.

FIG. 7B is a histogram of AlphaSeq™ affinity values for the replicates of wild-type mouse IL-21 binding to mouse IL-21R in pH 5 media.

FIG. 7C is a histogram of AlphaSeq™ affinity values for the replicates of wild-type mouse IL-21 binding to mouse IL-21R in pH 6 media.

FIG. 7D is a histogram of AlphaSeq™ affinity values for the replicates of wild-type mouse IL-21 binding to mouse IL-21R in pH 7 media.

FIG. 8A is a histogram of AlphaSeq™ affinity values for the full mouse IL-21 SSM binding to mouse IL-21R in unbuffered media.

FIG. 8B is a histogram of AlphaSeq™ affinity values for the full mouse IL-21 SSM binding to mouse IL-21R in pH 5 media.

FIG. 8C is a histogram of AlphaSeq™ affinity values for the full mouse IL-21 SSM binding to mouse IL-21R in pH 6 media.

FIG. 8D is a histogram of AlphaSeq™ affinity values for the full mouse IL-21 SSM binding to mouse IL-21R in pH 7 media.

FIG. 9A is a scatter plot showing the relationship between AlphaSeq™ affinity to mouse IL-21R (x-axis) and AlphaSeq™ affinity to rat IL-21R (y-axis) for each variant in the mouse IL-21 SSM library, measured in pH 7 media (left) and unbuffered media (right).

FIG. 9B is a scatter plot showing the relationship between AlphaSeq™ affinity to mouse IL-21R (x-axis) and AlphaSeq™ affinity to human IL-21R (y-axis) for each variant in the mouse IL-21 SSM library, measured in pH 7 media (left) and unbuffered media (right).

FIG. 9C is a scatter plot showing the relationship between AlphaSeq™ affinity to mouse IL-21R (x-axis) and AlphaSeq™ affinity to cynomolgus monkey IL-21R (y-axis) for each variant in the mouse IL-21 SSM library, measured in pH 7 media (left) and unbuffered media (right).

FIG. 10A is a histogram of AlphaSeq™ affinity values for the replicates of wild-type human IL-21 binding to human IL-21R in unbuffered media. These results serve as controls for the experiment in which stabilized IL-21 variants were identified, the results of which are shown in FIG. 10B.

FIG. 10B is a histogram of AlphaSeq™ affinity values for a library of double, triple, quadruple, and quintuple mutant variants of human IL-21 binding to human IL-21R in unbuffered media.

FIG. 11 shows BLI binding affinities for the interaction between human IL-21R and Fc fusions of wild-type human IL-21 and the three stabilized human IL-21 variants, IL-21_3610, IL-21_4177, and IL-21_5mut.

FIG. 12 shows the potency of wild-type human IL-21 and the three stabilized human IL-21 variants, IL-21_3610, IL-21_4177, and IL-21_5mut, measured as the EC50 for phosphorylation of STAT3 in a human PBMC Phosflow assay.

FIG. 13A is a histogram of AlphaSeq™ affinity values for the replicates of wild-type human IL-21 binding to human IL-21R in unbuffered media. These results serve as controls for the experiment in which combinations of stabilizing mutations were tested, the results of which are shown in FIG. 13B.

FIG. 13B is a histogram of AlphaSeq™ affinity values for a library of double, triple, and quadruple mutant variants of human IL-21 binding to human IL-21R in unbuffered media. These mutants were designed as combinations of substitutions that had stabilizing effects.

FIG. 14A is a histogram of expression values for replicates of parental human IL-21_0910. A value of 3 is the maximum possible expression in this assay and a value of 1 is the minimum possible expression.

FIG. 14B is a histogram of expression values for replicates of parental human IL-21_3610. A value of 3 is the maximum possible expression in this assay and a value of 1 is the minimum possible expression.

FIG. 14C is a histogram of expression values for replicates of parental human IL-21_4177. A value of 3 is the maximum possible expression in this assay and a value of 1 is the minimum possible expression.

FIG. 14D is a histogram of expression values for replicates of parental human IL-21_5474. A value of 3 is the maximum possible expression in this assay and a value of 1 is the minimum possible expression.

FIG. 14E is a histogram of expression values for replicates of parental human IL-21_5707. A value of 3 is the maximum possible expression in this assay and a value of 1 is the minimum possible expression.

FIG. 14F is a histogram of expression values for replicates of parental human IL-21_5845. A value of 3 is the maximum possible expression in this assay and a value of 1 is the minimum possible expression.

FIG. 14G is a histogram of expression values for replicates of parental human IL-21_6018. A value of 3 is the maximum possible expression in this assay and a value of 1 is the minimum possible expression.

FIG. 14H is a histogram of expression values for replicates of parental human IL-21_6318. A value of 3 is the maximum possible expression in this assay and a value of 1 is the minimum possible expression.

FIG. 15A is a histogram of expression values for SSM variants of human IL-21_0910. A value of 3 is the maximum possible expression in this assay and a value of 1 is the minimum possible expression.

FIG. 15B is a histogram of expression values for SSM variants of human IL-21_3610. A value of 3 is the maximum possible expression in this assay and a value of 1 is the minimum possible expression.

FIG. 15C is a histogram of expression values for SSM variants of human IL-21_4177. A value of 3 is the maximum possible expression in this assay and a value of 1 is the minimum possible expression.

FIG. 15D is a histogram of expression values for SSM variants of human IL-21_5474. A value of 3 is the maximum possible expression in this assay and a value of 1 is the minimum possible expression.

FIG. 15E is a histogram of expression values for SSM variants of human IL-21_5707. A value of 3 is the maximum possible expression in this assay and a value of 1 is the minimum possible expression.

FIG. 15F is a histogram of expression values for SSM variants of human IL-21_5845. A value of 3 is the maximum possible expression in this assay and a value of 1 is the minimum possible expression.

FIG. 15G is a histogram of expression values for SSM variants of human IL-21_6018. A value of 3 is the maximum possible expression in this assay and a value of 1 is the minimum possible expression.

FIG. 15H is a histogram of expression values for SSM variants of human IL-21_6318. A value of 3 is the maximum possible expression in this assay and a value of 1 is the minimum possible expression.

FIG. 16A is a histogram of AlphaSeq™ affinity values for the replicates of parental IL-21_0910 binding to human IL-21R in unbuffered media.

FIG. 16B is a histogram of AlphaSeq™ affinity values for the replicates of parental IL-21_3610 binding to human IL-21R in unbuffered media.

FIG. 16C is a histogram of AlphaSeq™ affinity values for the replicates of parental IL-21_4177 binding to human IL-21R in unbuffered media.

FIG. 16D is a histogram of AlphaSeq™ affinity values for the replicates of parental IL-21_5474 binding to human IL-21R in unbuffered media.

FIG. 16E is a histogram of AlphaSeq™ affinity values for the replicates of parental IL-21_5707 binding to human IL-21R in unbuffered media.

FIG. 16F is a histogram of AlphaSeq™ affinity values for the replicates of parental IL-21_5845 binding to human IL-21R in unbuffered media.

FIG. 16G is a histogram of AlphaSeq™ affinity values for the replicates of parental IL-21_6018 binding to human IL-21R in unbuffered media.

FIG. 16H is a histogram of AlphaSeq™ affinity values for the replicates of parental IL-21_6318 binding to human IL-21R in unbuffered media.

FIG. 17A is a histogram of AlphaSeq™ affinity values for the SSM variants of IL-21_0910 binding to human IL-21R in unbuffered media.

FIG. 17B is a histogram of AlphaSeq™ affinity values for the SSM variants of IL-21_3610 binding to human IL-21R in unbuffered media.

FIG. 17C is a histogram of AlphaSeq™ affinity values for the SSM variants of IL-21_4177 binding to human IL-21R in unbuffered media.

FIG. 17D is a histogram of AlphaSeq™ affinity values for the SSM variants of IL-21_5474 binding to human IL-21R in unbuffered media.

FIG. 17E is a histogram of AlphaSeq™ affinity values for the SSM variants of IL-21_5707 binding to human IL-21R in unbuffered media.

FIG. 17F is a histogram of AlphaSeq™ affinity values for the SSM variants of IL-21_5845 binding to human IL-21R in unbuffered media.

FIG. 17G is a histogram of AlphaSeq™ affinity values for the SSM variants of IL-21_6018 binding to human IL-21R in unbuffered media.

FIG. 17H is a histogram of AlphaSeq™ affinity values for the SSM variants of IL-21_6318 binding to human IL-21R in unbuffered media.

FIG. 18 is a bar plot showing % tumor growth inhibition for CD8-localized IL-21 molecules tested in an MC38 tumor model. % tumor growth inhibition was calculated using the formula (1−(mean tumor volume treatment group/mean tumor volume control group))*100 on the last day in which all vehicle control mice were on study (day 9). N=10 mice per group.

FIG. 19 shows survival curves for control and treatment groups in an MC38 tumor model study. Data is shown through day 32 of the study. N=10 mice per group.

### DETAILED DESCRIPTION

The present disclosure provides human interleukin 21 (IL-21) variants and fusion proteins including those variants. In some embodiments, the IL-21 variants are detuned IL-21 variants. “Detuned” means that the wildtype IL-21 is engineered to have a reduced or weakened affinity for the interleukin 21 receptor (IL-21R). Different modifications of the wildtype IL-21 results in a large number of detuned IL-21 variants.

Also described herein are IL-21 variants that exhibit increased stability, either expressed alone or in the context of a fusion protein. Given the biophysical liabilities of IL-21, stabilization of an IL-21 therapeutic is desirable. Stabilization can include various parameters and be measured in by various methods known in the art. Increased stability can include higher expression titers in cells, e.g., CHO cells, during manufacturing; increased monomeric species of interest measured by analytical size exclusion chromatography (SEC) following purification; or higher melting temperature in differential scanning fluorimetry (DSF). Additionally, stabilized can have longer half-lives in serum stability assays in vitro or enhanced pharmacokinetic properties when administered to an animal or patient as a drug.

The IL-21 variants disclosed herein can be detuned, exhibit increased stability, or both. In a therapeutic context, these IL-21 variants can be targeted to particular types of target cells, e.g., types of immune cells, of interest by linking, e.g., covalently linking, the detuned variants to an antibody or portion thereof against a specific cell surface marker. By reducing affinity for the receptor, the activity of the detuned cytokines is decreased for other cell types, e.g., in healthy tissues, which, in turn, decreases potential toxicity of the molecule when administered as a therapy. However, at the surface of the cell type targeted by the antibody, the residual activity of the cytokine is sufficient to bind its receptor and activate pro-inflammatory pathways leading to immune activation.

Cytokine signaling can trigger multifaceted and even opposing activities in different cell types, meaning that even if cytokine activity is limited specifically to the cells expressing the antibody, targeted antigen activation of other cell types may be detrimental to the therapeutic efficacy of the cytokine. Targeting cytokine-dependent activation to only a subset of the immune cells present in a tumor, for example, activated T cells as opposed to all cells with a cytokine receptor, can allow improved targeting of the therapeutic effects of the molecule.

The compositions disclosed herein are human IL-21 variants and fusion proteins made from these variants. As measured by, e.g., AlphaSeq™, and demonstrated in further detail in the Examples below, the IL-21 variants of the compositions and methods disclosed herein have decreased or no binding to the IL-21R receptor, as compared to the wild-type human IL-21 polypeptide or a wild-type IL-21 fusion polypeptide. In some embodiments, these reduced affinity IL-21 variants, when presented as an antibody fusion chimeric protein, are targeted selectively to desired cell types (those cells expressing the antibody target). Cell types that express the IL-21R receptor complex, but not the antibody target, are activated at a reduced level, or not activated at all, compared to those cells that express both components.

Accordingly, the IL-21 variants and the IL-21 fusion proteins of the compositions and methods disclosed herein selectively modulate the activation of cell subsets to promote biological activity, such as an anti-tumor activity, efficaciously and safely.

### Methods of Determining IL-21 Variants

In some embodiments, measuring the affinity of the cytokine IL-21 to its receptor IL-21R is performed using a high-throughput synthetic yeast agglutination protein-protein interaction (PPI) screening platform termed AlphaSeq™. Synthetic yeast agglutination relies on reprogramming yeast sexual agglutination—a naturally-occurring protein-protein interaction—to link protein-protein interaction strength with mating efficiency between a-type recombinant haploid yeast cells and α-type recombinant haploid yeast cells in liquid culture. For a screen of IL-21 variants for binding affinity to IL-21R based on synthetic yeast agglutination, mating efficiency, represented by the number of diploid yeast cells formed in a turbulent liquid culture, is a proxy for affinity between IL-21 and IL-21R.

The AlphaSeq™ method is disclosed in, e.g., U.S. Pat. Nos. 10,988,759 and 11,136,573, which are incorporated herein by reference in their entireties. AlphaSeq™ can be used to perform library-by-library screens of a library of variants of one protein binding partner, e.g., IL-21, against a library of variants of another protein binding partner, e.g., IL-21R. AlphaSeq™ can be also used to perform a library-based screen of a library of variants of one protein binding partner, e.g., IL-21, against a single species of another protein binding partner, e.g., wild-type human IL-21R or wild-type mouse IL-21R. Each variant, i.e., protein of interest (POI) is assigned a unique oligonucleotide molecular barcode, and after diploid formation events, these protein-specific barcodes can be recombined and sequenced to identify the individual synthetic adhesion proteins (SAPs) that mediated the corresponding diploid formation event. Quantifying sequencing reads of unique barcode-barcode combinations acts as a proxy measure of the number of diploid formation events, and thus, PPI affinity.

In native yeast sexual agglutination, in a turbulent liquid culture, MATa and MATα haploid cells stick to one another due to the binding of sexual agglutinin proteins, which allows them to mate. The native sexual agglutinin proteins consist of Agal and Aga2, expressed by MATa cells, and Sagl, expressed by MATα cells. Agal and Sagl form GPI anchors with the cell wall and extend outside of the cell wall with glycosylated stalks. Aga2 is secreted by MATa cells and forms a disulfide bond with Agal. The interaction between Aga2 and Sag1 is essential for wild-type sexual agglutination.

The native sexual agglutinin interaction can be replaced with an engineered one by expressing Agal in both mating types and fusing complementary binders to Aga2. In this case, a synthetic adhesion protein (SAP) comprises the fusion of Aga2 and the binder of interest, e.g., the library of IL-21 variants expressed by cells of one mating type and IL-21R expressed by cells of the other mating type. Interaction of the SAPs therefore mediates adhesion, and subsequently the agglutination process. In some embodiments, instead of direct agglutination, it may be possible to express SAPs for a multivalent target, such that agglutination and mating only occurs in the presence of the target.

In some embodiments, each MATa and MATα haploid cell contains an SAP fused to Aga2 integrated into a target chromosome (for example, chromosome III). Upon mating, both copies of the target chromosome are present in the same diploid cell. In addition to the SAP/Aga2 cassette, each copy of the target chromosome has a unique primer binding site, one of a plurality of unique oligonucleotide barcodes operably linked to the particular SAP, and a lox recombination site.

The plurality of oligonucleotide barcodes can be synthesized and assembled with the library of SAP expression cassettes such that a single SAP species is operably linked to a plurality of unique oligonucleotide barcodes. Upon expression of Cre recombinase, a chromosomal translocation occurs at the lox sites, resulting in a juxtaposition of the primer binding sites and barcodes onto the same copy of the target chromosome. A PCR is then performed to amplify a region of the chromosome containing the barcodes from both SAPs, such that sequences comprising unique barcode-barcode pairs, each representing a diploid formation event, are amplified. In a batched mating, the result is a pool of fragments, each containing the unique barcode-barcode pair associated with two SAPs that were responsible for the single diploid formation event. Paired-end next generation sequencing is then used to match the barcodes and determine the number of diploid formation events mediated by that SAP pair.

In some embodiments, the a-agglutinin, Sag1, is knocked out in MATa cells to eliminate native agglutination. MATa and MATα cells are unable to synthesize lysine or leucine, respectively. Diploids can then be selected for in media lacking both amino acids. MATa cells express ZEV4, a PE inducible transcription factor that activates Cre recombinase expression in diploid cells. MATa and MATα cells express mCherry and mTurquoise, respectively, for identification of strain types with flow cytometry. MATa and MATα cells constitutively express Agal along with a uniquely barcoded SAP fused to Aga2. When Cre recombinase expression is induced in diploids with PE, a chromosomal translocation at lox sites consolidates both SAP-Aga2 fusion expression cassettes onto the same chromosome. A single fragment containing the unique barcode-barcode sequence associated with that diploid formation event is then amplified by PCR with primers annealing to Pf and Pr (primers specific to the primers from the first and second nucleic acid constructs integrated at the genomic target site) and sequenced to quantify the number of diploid formation events and identify the interacting SAP pair.

In some embodiments, a CRE recombinase translocation scheme is utilized for high throughput analysis for interactions between synthetic adhesion proteins from a library-to-library screen, or, e.g., the library of IL-21 variants expressed by cells of one mating type and IL-21R expressed by cells of the other mating type. When CRE recombinase expression is induced in diploids with PE, a chromosomal translocation at lox sites consolidates both SAP-Aga2 expression cassettes onto the same chromosome. A single fragment containing the unique barcode-barcode sequence associated with that diploid formation event is then amplified by PCR with primers annealing to primer binding sites from each of the first and second nucleic acid constructs and sequenced (for example, using a paired end analysis of next generation sequencing) to quantify the number of diploid formation events and identify the interacting SAP pair, e.g., a unique variant of IL-21 and IL-21R, thereby yielding an estimation of the affinity between the variant of IL-21 and IL-21R.

In some embodiments, the methods for measuring the affinity of a cytokine and its receptor include a library of cytokine variants, e.g., variants of IL-21, comprising a plurality of cytokine variants and a receptor or library of receptor variants, e.g., IL-21R or variants of IL-21R. The cytokine variants and the receptor variants or library of receptor variants can be user-designated or randomly added mutants of a protein and the wild-type protein. In some embodiments, the amino acid substitutions may be generated by site saturation mutagenesis (SSM) to produce an SSM library of the cytokine and the receptor. In some embodiments, the variants and the receptor variants can be generated by alanine scanning. In some embodiments, the cytokine variants and the receptor variants can be generated by random mutagenesis, such as with error prone PCR, or another method to introduce variation into the amino acid sequence of the expressed protein. The cytokine variant library comprising a plurality of cytokine variants and the receptor variant library comprising a plurality of receptor variants are assayed for binding affinity, such that affinity is measured for interaction between each of the plurality of variants and the wild-type receptor or each of the plurality of receptor variants individually, in a parallelized high-throughput manner.

In some embodiments, AlphaSeq™ can be used to measure affinities in various buffer conditions, including for example unbuffered media, pH 5, pH 6, and pH 7 buffered media. Performing the AlphaSeq™ assay in culture media that is buffered to a lower pH can result in an increased dynamic range for detecting interactions between a cytokine and its receptor, and therefore, increased accuracy and resolution for detecting affinities between cytokine variants and the receptor. Affinities for IL-21 variants comprising single amino acid substitutions with detuned affinity to human IL-21R measured in each of several pH conditions are provided in Table 1.

In some embodiments, the cytokine variants and the receptor or receptor variants are full-length proteins. In some embodiments, the cytokine variants and the receptor or receptor variants are truncated proteins. In some embodiments, the cytokine variants and the receptor or receptor variants are fusion proteins. In some embodiments, the cytokine variants and the receptor or receptor variants are tagged proteins. Tagged proteins include proteins that are epitope tagged, e.g., FLAG-tagged, HA-tagged, His-tagged, Myc-tagged, among others known in the art. The cytokine variants and the receptor or receptor variants can each be any of the following: a full-length protein, truncated protein, fusion protein, tagged protein, or combinations thereof.

In some embodiments, surface expression of each displayed IL-21 variant is measured to select for higher expressing variants to ensure that displayed variant proteins are folded, functional variants and not poorly displayed proteins. In some embodiments, the library of cytokine variants is labeled with a FITC-conjugated anti-HA antibody. Labeled cells are then sorted by FACS into one of three bins—low (bin 1), medium (bin 2), or high (bin 3)—based on fluorescence intensity.

In some embodiments, the methods for measuring the affinity of a cytokine and its receptor include bio-layer interferometry (BLI). BLI measures kinetics and biomolecular interactions on a basis of wave interference. To prepare for BLI analysis between two unique biomolecules, the ligand is first immobilized onto a biocompatible biosensor while the analyte is in solution. After this, the biosensor tip is dipped into the solution and the target molecule will begin to associate with the analyte, producing a layer on top of the biosensor tip. This creates two separate surfaces: the substrate itself, and the substrate interacting with the molecule immobilized on the biosensor tip. This can create a thin-film interference, in which the created layer acts as a thin film bound by these two surfaces. White light from a tungsten lamp is shone onto the biosensor tip and reflected off both surfaces, creating two unique reflection patterns with different intensities.

In some cases, it is possible that cytokine variants, e.g., IL-21 variants, are measured by various affinity determination methods, e.g., AlphaSeq™ and/or BLI to have no detectable or measurable binding to the cytokine's receptor, e.g., IL-21R, while still inducing signaling in cells in vitro or in vivo. In such cases, a cytokine variant exhibiting no binding to its receptor as measured by AlphaSeq™ or BLI, or a fusion protein comprising the cytokine variant, may still have some therapeutic efficacy.

### IL-21 Variants

IL-21 variants (e.g., human IL-21 variants) as described herein have decreased or no binding to the IL-21R receptor and/or reduced interaction between IL-21 and the IL-21R receptor, as compared to the wild-type human IL-21 polypeptide, e.g., as described in further detail in the Examples section. The IL-21 variants provided herein are recited in Tables 1-3 (below) as including amino acid substitutions numbered according to the wild-type sequence of human IL-21 and mouse IL-21, respectively. The wild-type amino acid sequence of human IL-21 is provided as SEQ ID NO: 1 below:

(SEQ ID NO: 1)
MRSSPGNMERIVICLMVIFLGTLVHKSSSQGQDRHMIRMRQLIDI
VDQLKNYVNDLVPEFLPAPEDVETNCEWSAFSCFQKAQLKSANTG
NNERIINVSIKKLKRKPPSTNAGRRQKHRLTCPSCDSYEKKPPKE
FLERFKSLLQKMIHQHLSSRTHGSEDS

For example, a variant with the name “IL-21_Y52D” has an amino acid substitution in SEQ ID NO: 1 at location 52, where the existing tyrosine is replaced with aspartic acid.

IL-21 has a four-helix bundle structure and exists as a monomer. In humans, two isoforms of IL-21 are known, each of which are derived from a precursor molecule. The first IL-21 isoform comprises 162 amino acids (aa), the first 29 of which make up the signal peptide; and the second IL-21 isoform comprises 153 aa, the first 29 of which make up the signal peptide as in the first isoform. IL-21 as disclosed herein are numbered according to the full-length first isoform including the signal peptide, i.e., an 162 aa polypeptide.

Tables 1 and 3 also recite affinities between IL-21 and IL-21R as measured by AlphaSeq™ (in nM). The amino acid sequence of wild-type human IL-21R is provided as SEQ ID NO: 2 below:

(SEQ ID NO: 2)
MPRGWAAPLLLLLLQGGWGCPDLVCYTDYLQTVICILEMWNLHPS
TLTLTWQDQYEELKDEATSCSLHRSAHNATHATYTCHMDVFHFMA
DDIFSVNITDQSGNYSQECGSFLLAESIKPAPPENVTVTFSGQYN
ISWRSDYEDPAFYMLKGKLQYELQYRNRGDPWAVSPRRKLISVDS
RSVSLLPLEFRKDSSYELQVRAGPMPGSSYQGTWSEWSDPVIFQT
QSEELKEGWNPHLLLLLLLVIVFIPAFWSLKTHPLWRLWKKIWAV
PSPERFFMPLYKGCSGDFKKWVGAPFTGSSLELGPWSPEVPSTLE
VYSCHPPRSPAKRLQLTELQEPAELVESDGVPKPSFWPTAQNSGG
SAYSEERDRPYGLVSIDTVTVLDAEGPCTWPCSCEDDGYPALDLD
AGLEPSPGLEDPLLDAGTTVLSCGCVSAGSPGLGGPLGSLLDRLK
PPLADGEDWAGGLPWGGRSPGGVSESEAGSPLAGLDMDTFDSGFV
GSDCSSPVECDFTSPGDEGPPRSYLRQWVVIPPPLSSPGPQAS

In some embodiments, provided herein are variants of mouse IL-21 that have decreased or no binding to the mouse IL-21R receptor and/or reduced interaction between mouse IL-21 and the mouse IL-21R receptor, as compared to the wild-type mouse IL-21 polypeptide, as described in further detail in the Examples section. The amino acid sequence of wild-type mouse IL-21 is provided as SEQ ID NO: 3 below:

(SEQ ID NO: 3)
MERTLVCLVVIFLGTVAHKSSPQGPDRLLIRLRHLIDIVEQLKIY
ENDLDPELLSAPQDVKGHCEHAAFACFQKAKLKPSNPGNNKTFII
DLVAQLRRRLPARRGGKKQKHIAKCPSCDSYEKRTPKEFLERLKW
LLQKMIHQHLS

The amino acid sequence of wild-type mouse IL-21R is provided as SEQ ID NO: 4 below:

(SEQ ID NO: 4)
MPRGPVAALLLLILHGAWSCLDLTCYTDYLWTITCVLETRSPNPS
ILSLTWQDEYEELQDQETFCSLHRSGHNTTHIWYTCHMRLSQFLS
DEVFIVNVTDQSGNNSQECGSFVLAESIKPAPPLNVTVAFSGRYD
ISWDSAYDEPSNYVLRGKLQYELQYRNLRDPYAVRPVTKLISVDS
RNVSLLPEEFHKDSSYQLQVRAAPQPGTSFRGTWSEWSDPVIFQT
QAGEPEAGWDPHMLLLLAVLIIVLVEMGLKIHLPWRLWKKIWAPV
PTPESFFQPLYREHSGNFKKWVNTPFTASSIELVPQSSTTTSALH
LSLYPAKEKKFPGLPGLEEQLECDGMSEPGHWCIIPLAAGQAVSA
YSEERDRPYGLVSIDTVTVGDAEGLCVWPCSCEDDGYPAMNLDAG
RESGPNSEDLLLVTDPAFLSCGCVSGSGLRIGGSPGSLLDRLRLS
FAKEGDWTADPTWRTGSPGGGSESEAGSPPGLDMDTFDSGFAGSD
CGSPVETDEGPPRSYLRQWVVRTPPPVDSGAQSS

Table 1 below discloses 273 human IL-21 variants comprising single amino acid substitutions, with the single amino acid substitutions occurring at locations throughout the length of the wild-type human IL-21 polypeptide (SEQ ID NO: 1) with reduced affinity for human IL-21R (SEQ ID NO: 2). Any variant found to bind human IL-21R with a half-log weaker affinity than wild-type human IL-21 as measured by AlphaSeq™ (cutoff=2.0 μM in unbuffered media, 56 nM in pH 7 media, 41 nM in pH 6 media, and 94 nM in pH 5 media) and found to have an average expression bin>2.25 is included in Table 1. Table 1 is sorted in order of descending K_{d} in the unbuffered condition, with the highest values representing the lowest affinity of human IL-21 for human IL-21R.

TABLE 1
IL-21 Variants Comprising Single Amino Acid Substitutions
with Detuned Affinity to Human IL21R

| | Average | Affinity, | Affinity, | Affinity, | Affinity, |
|---|---|---|---|---|---|
| | expres- | pH 5 | pH 6 | pH 7 | unbuffered |
| Variant | sion bin | (nM) | (nM) | (nM) | (nM) |
| IL-21_Y52D | 2.46 | 3.8E+05 | 2.2E+03 | 6.1E+02 | 1.3E+06 |
| IL-21_V46Q | 2.47 | 4.0E+05 | 2.6E+05 | 7.1E+04 | 1.0E+06 |
| IL-21_W73K | 2.5 | 2.0E+05 | 3.0E+04 | 1.0E+04 | 8.9E+05 |
| IL-21_M39K | 2.9 | 1.9E+05 | 1.3E+04 | 2.2E+04 | 8.9E+05 |
| IL-21_M39D | 2.41 | 2.8E+05 | 7.6E+04 | NA | 8.0E+05 |
| IL-21_A75N | 2.47 | 2.1E+05 | 2.8E+04 | NA | 7.8E+05 |
| IL-21_V46K | 2.5 | 3.4E+05 | NA | 2.8E+04 | 7.3E+05 |
| IL-21_L56S | 2.68 | 3.1E+05 | 4.6E+04 | 4.4E+04 | 6.8E+05 |
| IL-21_Q80D | 2.32 | 5.8E+05 | NA | NA | 6.6E+05 |
| IL-21_V46T | 2.44 | 2.2E+04 | 7.1E+01 | 5.3E+01 | 6.4E+05 |
| IL-21_V57H | 2.55 | 1.6E+03 | 2.4E+01 | 1.9E+01 | 6.1E+05 |
| IL-21_F76Q | 2.25 | 3.1E+05 | 5.0E+04 | 6.3E+04 | 6.1E+05 |
| IL-21_M39Q | 2.36 | 2.2E+05 | 8.3E+04 | NA | 6.1E+05 |
| IL-21_D44P | 2.39 | 2.0E+05 | 7.3E+03 | 1.8E+03 | 5.9E+05 |
| IL-21_L103E | 2.32 | 2.4E+05 | 9.2E+04 | NA | 5.9E+05 |
| IL-21_I96D | 2.41 | 3.9E+05 | 6.0E+04 | 3.5E+04 | 5.8E+05 |
| IL-21_V53N | 2.34 | 1.8E+05 | 3.7E+03 | 9.9E+02 | 5.6E+05 |
| IL-21_L42H | 2.47 | 2.2E+05 | 6.7E+04 | 5.4E+03 | 5.5E+05 |
| IL-21_LA9E | 2.47 | 6.0E+05 | NA | NA | 5.4E+05 |
| IL-21_D47P | 2.27 | 1.3E+05 | 2.7E+04 | NA | 5.3E+05 |
| IL-21_I100E | 2.41 | 1.2E+05 | 7.8E+04 | NA | 5.1E+05 |
| IL-21_V46N | 2.41 | 3.7E+05 | 3.7E+04 | NA | 5.1E+05 |
| IL-21_R105Q | 2.37 | 2.0E+05 | NA | NA | 4.9E+05 |
| IL-21_V46G | 2.47 | 4.3E+05 | NA | NA | 4.9E+05 |
| IL-21_F76P | 2.31 | 2.5E+05 | 4.1E+04 | 3.8E+04 | 4.8E+05 |
| IL-21_W73V | 2.26 | 1.1E+05 | 6.5E+03 | 9.2E+02 | 4.8E+05 |
| IL-21_I43P | 2.54 | 3.3E+05 | 6.1E+04 | NA | 4.7E+05 |
| IL-21_Q80A | 2.25 | 2.1E+05 | 1.2E+04 | 2.4E+04 | 4.6E+05 |
| IL-21_R105D | 2.39 | 1.7E+05 | NA | NA | 4.6E+05 |
| IL-21_F76H | 2.26 | 7.9E+04 | 1.4E+04 | 9.2E+03 | 4.6E+05 |
| IL-21_LA2E | 2.56 | 1.9E+05 | NA | 2.6E+04 | 4.5E+05 |
| IL-21_W73T | 2.43 | 1.3E+05 | 4.1E+03 | 7.9E+02 | 4.5E+05 |
| IL-21_F79P | 2.36 | 1.3E+05 | 7.8E+03 | 3.3E+03 | 4.4E+05 |
| IL-21_LA2G | 2.44 | 2.1E+05 | 3.2E+04 | 2.4E+04 | 4.2E+05 |
| IL-21_I43D | 2.34 | 1.3E+05 | NA | 4.0E+03 | 4.2E+05 |
| IL-21_P108T | 2.51 | 5.7E+04 | 1.9E+03 | 1.1E+03 | 4.2E+05 |
| IL-21_Q41K | 2.6 | 1.5E+05 | NA | 7.2E+03 | 4.2E+05 |
| IL-21_LA9H | 2.34 | 1.6E+05 | NA | NA | 4.1E+05 |
| IL-21_F76R | 2.28 | 1.3E+05 | NA | NA | 4.1E+05 |
| IL-21_V46E | 2.41 | 3.5E+05 | NA | NA | 4.0E+05 |
| IL-21_R38V | 2.63 | 3.5E+05 | NA | NA | 3.8E+05 |
| IL-21_LA9D | 2.68 | 4.8E+05 | NA | NA | 3.7E+05 |
| IL-21_F76D | 2.45 | 2.2E+05 | 5.5E+04 | NA | 3.7E+05 |
| IL-21_W73S | 2.34 | 3.9E+04 | 2.3E+03 | 3.4E+02 | 3.5E+05 |
| IL-21_V57P | 2.32 | 6.8E+05 | 1.9E+04 | 3.0E+03 | 3.5E+05 |
| IL-21_M39R | 2.52 | 2.8E+05 | 1.7E+05 | NA | 3.4E+05 |
| IL-21_Y52K | 2.34 | 1.8E+05 | 2.8E+03 | 4.9E+02 | 3.4E+05 |
| IL-21_F76T | 2.26 | 7.5E+04 | NA | NA | 3.4E+05 |
| IL-21_F79Y | 2.29 | 7.2E+04 | 2.0E+03 | 5.3E+02 | 3.3E+05 |
| IL-21_M39E | 2.47 | 1.5E+05 | 4.2E+04 | NA | 3.3E+05 |
| IL-21_I45G | 2.61 | 2.0E+05 | 5.0E+03 | 1.2E+03 | 3.3E+05 |
| IL-21_D47S | 2.36 | 7.4E+05 | 4.6E+03 | 5.0E+02 | 3.3E+05 |
| IL-21_F79K | 2.29 | 7.4E+04 | 5.5E+03 | 1.8E+03 | 3.1E+05 |
| IL-21_LA9K | 2.45 | 2.0E+05 | NA | NA | 3.1E+05 |
| IL-21_L42Q | 2.35 | 3.2E+05 | 4.9E+03 | 4.6E+02 | 3.1E+05 |
| IL-21_I45H | 2.67 | 2.2E+05 | 6.2E+03 | 6.4E+02 | 3.1E+05 |
| IL-21_LA9N | 2.61 | 1.7E+05 | NA | NA | 3.0E+05 |
| IL-21_I45R | 2.55 | 3.7E+05 | 2.9E+04 | NA | 3.0E+05 |
| IL-21_I37R | 2.43 | 8.7E+02 | 1.7E+02 | 2.0E+02 | 3.0E+05 |
| IL-21_LA2N | 2.42 | 2.8E+05 | 2.6E+04 | 9.5E+02 | 3.0E+05 |
| IL-21_R105S | 2.3 | 1.3E+05 | NA | NA | 3.0E+05 |
| IL-21_I43S | 2.48 | 4.3E+04 | 1.1E+03 | 2.9E+02 | 3.0E+05 |
| IL-21_LA9P | 2.64 | 3.3E+05 | 5.5E+04 | NA | 2.9E+05 |
| IL-21_I95P | 2.32 | 6.4E+04 | 3.6E+03 | 7.0E+02 | 2.9E+05 |
| IL-21_R38A | 2.32 | 1.5E+04 | 1.7E+03 | NA | 2.8E+05 |
| IL-21_I45S | 2.74 | 1.5E+04 | 1.1E+02 | 1.6E+02 | 2.8E+05 |
| IL-21_R105W | 2.34 | 3.2E+05 | NA | NA | 2.8E+05 |
| IL-21_V46P | 2.5 | 5.1E+05 | NA | NA | 2.8E+05 |
| IL-21_I45Y | 2.34 | 2.0E+05 | 1.2E+04 | 3.8E+03 | 2.8E+05 |
| IL-21_R38Q | 2.25 | 1.4E+05 | 1.2E+04 | 4.2E+03 | 2.7E+05 |
| IL-21_I96P | 2.27 | 1.4E+05 | 5.7E+03 | 3.0E+03 | 2.7E+05 |
| IL-21_F76E | 2.26 | 1.0E+05 | 6.9E+03 | 2.4E+04 | 2.7E+05 |
| IL-21_R105A | 2.38 | 2.6E+05 | NA | 2.0E+04 | 2.7E+05 |
| IL-21_V46D | 2.71 | 8.6E+04 | 1.2E+04 | NA | 2.7E+05 |
| IL-21_V53P | 2.26 | 2.7E+05 | 2.7E+04 | 4.0E+03 | 2.7E+05 |
| IL-21_M39P | 2.7 | NA | NA | NA | 2.6E+05 |
| IL-21_I95K | 2.38 | 1.9E+04 | 6.8E+02 | 2.4E+02 | 2.6E+05 |
| IL-21_LA2K | 2.47 | 1.3E+05 | NA | 1.2E+04 | 2.5E+05 |
| IL-21_R105K | 2.53 | 1.3E+05 | 6.2E+02 | 2.2E+02 | 2.5E+05 |
| IL-21_R38K | 2.36 | 2.3E+05 | 1.8E+03 | 7.0E+03 | 2.5E+05 |
| IL-21_M39G | 2.34 | 1.5E+05 | 3.1E+04 | 7.8E+03 | 2.4E+05 |
| IL-21_L56F | 2.28 | 5.4E+04 | 7.1E+02 | 2.4E+02 | 2.4E+05 |
| IL-21_I37S | 2.41 | 1.6E+04 | 5.3E+02 | 3.2E+02 | 2.4E+05 |
| IL-21_D44A | 2.34 | 1.4E+02 | 5.2E+01 | 2.4E+02 | 2.4E+05 |
| IL-21_I37N | 2.27 | 3.3E+04 | 6.8E+02 | 3.3E+02 | 2.3E+05 |
| IL-21_I96T | 2.26 | 2.1E+05 | 6.6E+04 | NA | 2.2E+05 |
| IL-21_L56N | 2.42 | 7.2E+04 | NA | 9.7E+03 | 2.1E+05 |
| IL-21_I45P | 2.67 | 1.0E+05 | NA | NA | 2.1E+05 |
| IL-21_I96G | 2.44 | 1.9E+05 | 5.2E+04 | NA | 2.1E+05 |
| IL-21_E59R | 2.28 | 4.1E+04 | 1.5E+03 | 3.4E+02 | 2.1E+05 |
| IL-21_Q41P | 2.35 | 1.8E+05 | 3.5E+04 | NA | 2.0E+05 |
| IL-21_I45D | 2.54 | 2.0E+04 | 1.9E+02 | 1.8E+02 | 1.9E+05 |
| IL-21_P108Q | 2.29 | 3.8E+03 | 3.0E+02 | 1.1E+02 | 1.9E+05 |
| IL-21_LA9S | 2.4 | 2.4E+05 | NA | NA | 1.9E+05 |
| IL-21_LA2D | 2.5 | 1.6E+06 | 1.4E+04 | 3.7E+03 | 1.9E+05 |
| IL-21_LA2S | 2.46 | 1.8E+05 | NA | 1.1E+04 | 1.9E+05 |
| IL-21_Q48K | 2.41 | 1.1E+04 | 2.0E+02 | 2.2E+02 | 1.9E+05 |
| IL-21_F79T | 2.28 | 9.2E+04 | 3.3E+03 | 1.4E+03 | 1.8E+05 |
| IL-21_I45N | 2.52 | 3.2E+05 | 3.8E+03 | 1.0E+03 | 1.8E+05 |
| IL-21_W73P | 2.59 | 4.4E+05 | 8.7E+03 | 7.3E+02 | 1.8E+05 |
| IL-21_D44G | 2.46 | 4.8E+02 | 5.0E+01 | 2.0E+02 | 1.8E+05 |
| IL-21_L120V | 2.68 | 1.2E+03 | 2.1E+02 | 2.1E+02 | 1.8E+05 |
| IL-21_I148S | 2.34 | 3.1E+02 | 4.2E+01 | 2.9E+02 | 1.7E+05 |
| IL-21_F79D | 2.32 | 5.2E+04 | 2.7E+03 | 6.4E+02 | 1.7E+05 |
| IL-21_I37G | 2.31 | 8.2E+04 | 8.6E+02 | 6.6E+02 | 1.7E+05 |
| IL-21_LA9R | 2.57 | 3.9E+05 | NA | NA | 1.6E+05 |
| IL-21_I96A | 2.44 | 4.2E+05 | 1.1E+04 | NA | 1.6E+05 |
| IL-21_I95H | 2.32 | 5.3E+04 | 2.0E+03 | 5.8E+02 | 1.5E+05 |
| IL-21_W73G | 2.41 | 5.7E+04 | 2.2E+03 | 3.3E+02 | 1.5E+05 |
| IL-21_V98K | 2.59 | 4.9E+02 | 9.4E+01 | 2.5E+02 | 1.5E+05 |
| IL-21_V53Q | 2.55 | 1.8E+04 | 1.6E+02 | 2.0E+02 | 1.4E+05 |
| IL-21_P108G | 2.44 | 1.1E+04 | 7.9E+02 | 4.7E+02 | 1.4E+05 |
| IL-21_D33P | 2.47 | 4.0E+04 | 7.7E+02 | 2.7E+02 | 1.3E+05 |
| IL-21_F79R | 2.33 | 6.5E+04 | 4.4E+03 | 4.5E+03 | 1.3E+05 |
| IL-21_W73N | 2.34 | 6.5E+04 | 3.4E+03 | 8.5E+02 | 1.2E+05 |
| IL-21_I96S | 2.35 | 4.6E+05 | 1.3E+04 | NA | 1.2E+05 |
| IL-21_R38M | 2.38 | 8.8E+02 | 1.2E+03 | 2.0E+03 | 1.2E+05 |
| IL-21_V98R | 2.52 | 4.6E+01 | 1.9E+01 | 2.8E+01 | 1.2E+05 |
| IL-21_P132A | 2.29 | 4.7E+02 | 2.4E+01 | 1.2E+02 | 1.2E+05 |
| IL-21_LA2R | 2.43 | 1.5E+05 | NA | NA | 1.2E+05 |
| IL-21_D33R | 2.69 | 2.6E+04 | 8.1E+02 | 1.4E+02 | 1.2E+05 |
| IL-21_Y52N | 2.7 | 2.4E+04 | 3.8E+02 | 2.4E+02 | 1.1E+05 |
| IL-21_L56E | 2.25 | 1.2E+06 | 3.0E+04 | NA | 1.1E+05 |
| IL-21_I37E | 2.61 | 1.5E+03 | 5.5E+02 | 7.2E+02 | 1.1E+05 |
| IL-21_R38E | 2.62 | NA | 8.4E+03 | NA | 1.1E+05 |
| IL-21_F79E | 2.35 | 5.9E+04 | 7.0E+03 | 6.9E+03 | 1.1E+05 |
| IL-21_P62V | 2.3 | 1.1E+04 | 2.7E+02 | 1.2E+02 | 1.1E+05 |
| IL-21_L56T | 2.6 | 2.3E+05 | 6.9E+03 | NA | 1.0E+05 |
| IL-21_I37K | 2.61 | 3.3E+03 | 1.9E+02 | 1.2E+02 | 9.5E+04 |
| IL-21_S74H | 2.51 | 1.7E+02 | 2.6E+01 | 3.1E+01 | 8.5E+04 |
| IL-21_Y52H | 2.51 | 4.4E+03 | 2.9E+02 | 1.1E+02 | 6.9E+04 |
| IL-21_D44T | 2.51 | 3.5E+02 | 4.4E+01 | 1.6E+02 | 6.8E+04 |
| IL-21_Y52Q | 2.42 | 5.5E+04 | 3.9E+02 | 8.9E+01 | 6.3E+04 |
| IL-21_D44N | 2.28 | 1.2E+03 | 9.2E+01 | 2.9E+02 | 6.0E+04 |
| IL-21_S99H | 2.35 | 3.7E+03 | 3.2E+01 | 2.5E+01 | 6.0E+04 |
| IL-21_V53H | 2.62 | 2.2E+03 | 3.8E+01 | 3.8E+01 | 4.6E+04 |
| IL-21_S77R | 2.55 | 1.9E+01 | 1.2E+01 | 3.2E+01 | 4.6E+04 |
| IL-21_S109K | 2.25 | 4.3E+01 | 2.6E+01 | 3.0E+01 | 4.0E+04 |
| IL-21_I95T | 2.48 | 9.8E+03 | 5.5E+02 | 1.9E+02 | 3.4E+04 |
| IL-21_W73L | 2.38 | 5.2E+03 | 2.0E+02 | 1.3E+02 | 3.3E+04 |
| IL-21_S74N | 2.3. | 4.8E+04 | 2.9E+02 | 4.1E+02 | 3.2E+04 |
| IL-21_Q145H | 2.27 | 1.0E+03 | 5.4E+01 | 6.8E+01 | 3.2E+04 |
| IL-21_V53E | 2.72 | 1.8E+02 | 2.2E+01 | 1.9E+01 | 3.1E+04 |
| IL-21_V53T | 2.84 | 9.6E+03 | 6.3E+01 | 1.8E+02 | 3.0E+04 |
| IL-21_K131M | 2.34 | 4.6E+02 | 6.9E+01 | 8.6E+01 | 2.7E+04 |
| IL-21_W73A | 2.37 | 3.5E+03 | 3.6E+02 | 1.7E+02 | 2.6E+04 |
| IL-21_R34S | 2.29 | 3.8E+03 | 3.0E+02 | 2.7E+02 | 2.3E+04 |
| IL-21_L120H | 2.56 | 3.4E+01 | 1.9E+01 | 3.0E+01 | 2.2E+04 |
| IL-21_N88K | 2.28 | 1.7E+02 | 4.3E+01 | 1.4E+02 | 2.1E+04 |
| IL-21_R34W | 2.47 | 6.6E+02 | 9.6E+01 | 1.9E+02 | 2.0E+04 |
| IL-21_D33A | 2.41 | 5.8E+02 | 3.8E+01 | 8.3E+01 | 1.9E+04 |
| IL-21_L61I | 2.38 | 3.7E+02 | 3.7E+01 | 8.3E+01 | 1.9E+04 |
| IL-21_I37Q | 2.65 | 2.5E+02 | 3.5E+01 | 1.4E+02 | 1.9E+04 |
| IL-21_R34D | 2.58 | 2.3E+04 | 8.6E+02 | 7.4E+02 | 1.8E+04 |
| IL-21_T89L | 2.51 | 1.0E+03 | 1.1E+02 | 1.4E+02 | 1.6E+04 |
| IL-21_D33T | 2.25 | 1.3E+02 | 3.8E+01 | 2.2E+02 | 1.5E+04 |
| IL-21_W73Q | 2.37 | 6.7E+02 | 8.8E+01 | 1.2E+02 | 1.5E+04 |
| IL-21_S77K | 2.29 | 1.9E+02 | 1.3E+02 | 1.7E+02 | 1.4E+04 |
| IL-21_R34K | 2.42 | 3.9E+02 | 4.7E+01 | 7.4E+01 | 1.4E+04 |
| IL-21_D33N | 2.32 | 1.2E+02 | 2.7E+01 | 4.5E+01 | 1.4E+04 |
| IL-21_V57R | 2.49 | 5.0E+01 | 1.6E+01 | 5.0E+01 | 1.4E+04 |
| IL-21_Q150R | 2.34 | 1.8E+02 | 5.0E+01 | 9.4E+01 | 1.4E+04 |
| IL-21_D33S | 2.52 | 8.2E+01 | 1.9E+01 | 2.7E+01 | 1.3E+04 |
| IL-21_E72S | 2.39 | 5.3E+02 | 1.4E+02 | 3.4E+02 | 1.3E+04 |
| IL-21_H118R | 2.37 | 2.0E+04 | 9.5E+02 | 2.6E+02 | 1.1E+04 |
| IL-21_S142H | 2.39 | 5.1E+01 | 1.9E+01 | 3.8E+01 | 1.1E+04 |
| IL-21_R34H | 2.51 | 1.9E+03 | 6.9E+01 | 2.0E+02 | 1.0E+04 |
| IL-21_R34G | 2.69 | 6.4E+03 | 1.5E+02 | 1.8E+02 | 9.9E+03 |
| IL-21_V98H | 2.52 | 1.7E+02 | 2.6E+01 | 2.2E+01 | 8.8E+03 |
| IL-21_L120K | 2.48 | 4.7E+01 | 2.5E+01 | 4.6E+01 | 8.7E+03 |
| IL-21_N51P | 2.63 | 1.6E+02 | 2.4E+01 | 4.7E+01 | 8.5E+03 |
| IL-21_I45A | 2.65 | 1.8E+02 | 2.5E+01 | 6.1E+01 | 8.4E+03 |
| IL-21_P108L | 2.26 | 3.6E+02 | 3.5E+01 | 9.9E+01 | 8.1E+03 |
| IL-21_N54K | 2.28 | 1.9E+02 | 2.9E+01 | 3.2E+01 | 8.0E+03 |
| IL-21_A112R | 2.36 | 3.9E+01 | 1.9E+01 | 3.1E+01 | 7.9E+03 |
| IL-21_F60I | 2.44 | 3.4E+02 | 6.4E+01 | 1.9E+02 | 7.7E+03 |
| IL-21_N97G | 2.26 | 7.7E+01 | 2.4E+01 | 4.8E+01 | 7.6E+03 |
| IL-21_D33Q | 2.45 | 8.4E+01 | 2.1E+01 | 1.9E+01 | 7.5E+03 |
| IL-21_E93G | 2.36 | 7.3E+01 | 3.7E+01 | 1.5E+02 | 7.2E+03 |
| IL-21_H118K | 2.33 | 9.9E+04 | 3.1E+03 | 3.7E+02 | 6.8E+03 |
| IL-21_L56M | 2.44 | 3.5E+02 | 4.5E+01 | 8.4E+01 | 6.7E+03 |
| IL-21_P107Y | 2.49 | 1.2E+02 | 5.8E+01 | 2.2E+02 | 6.5E+03 |
| IL-21_E68R | 2.32 | 4.0E+01 | 1.6E+01 | 6.9E+01 | 6.3E+03 |
| IL-21_S109R | 2.32 | 3.6E+01 | 1.5E+01 | 1.4E+01 | 6.0E+03 |
| IL-21_N54P | 2.47 | 8.9E+01 | 2.0E+01 | 2.7E+01 | 5.4E+03 |
| IL-21_P62H | 2.43 | 1.6E+02 | 2.1E+01 | 1.1E+02 | 5.1E+03 |
| IL-21_E59S | 2.27 | 4.1E+01 | 1.6E+01 | 2.3E+01 | 4.9E+03 |
| IL-21_E72I | 2.5 | 4.2E+01 | 1.5E+01 | 1.3E+02 | 4.7E+03 |
| IL-21_P62T | 2.31 | 1.3E+02 | 2.8E+01 | 4.6E+01 | 4.5E+03 |
| IL-21_L152H | 2.47 | 2.6E+01 | 1.2E+01 | 3.0E+01 | 4.4E+03 |
| IL-21_S74A | 2.51 | 1.4E+02 | 3.8E+01 | 1.6E+02 | 4.4E+03 |
| IL-21_N70H | 2.26 | 6.4E+01 | 2.0E+01 | 2.2E+01 | 4.4E+03 |
| IL-21_F76M | 2.39 | 7.7E+01 | 2.1E+01 | 7.5E+01 | 4.3E+03 |
| IL-21_S86I | 2.42 | 1.1E+02 | 2.3E+01 | 5.2E+01 | 4.3E+03 |
| IL-21_F60R | 2.35 | 7.6E+01 | 2.6E+01 | 2.7E+01 | 4.2E+03 |
| IL-21_P108I | 2.36 | 1.8E+02 | 6.1E+01 | 7.1E+01 | 4.2E+03 |
| IL-21_N54H | 2.37 | 7.7E+01 | 2.0E+01 | 2.7E+01 | 4.1E+03 |
| IL-21_R34E | 2.63 | 8.7E+03 | 5.0E+02 | 2.1E+02 | 4.0E+03 |
| IL-21_A87N | 2.43 | 1.1E+02 | 2.5E+01 | 1.2E+02 | 3.9E+03 |
| IL-21_W73Y | 2.39 | 1.9E+02 | 8.5E+01 | 6.8E+01 | 3.7E+03 |
| IL-21_I43M | 2.3 | 1.1E+02 | 2.7E+01 | 4.9E+01 | 3.5E+03 |
| IL-21_E72H | 2.36 | 2.8E+01 | 1.1E+01 | 2.1E+01 | 3.4E+03 |
| IL-21_R34Y | 2.36 | 6.7E+03 | 3.5E+02 | 2.7E+02 | 3.4E+03 |
| IL-21_A63T | 2.54 | 6.4E+01 | 2.1E+01 | 2.1E+02 | 3.3E+03 |
| IL-21_E68K | 2.27 | 2.7E+01 | 1.4E+01 | 2.2E+01 | 3.3E+03 |
| IL-21_Q48G | 2.52 | 5.9E+01 | 1.6E+01 | 3.9E+01 | 3.2E+03 |
| IL-21_I37V | 2.64 | 5.8E+01 | 1.8E+01 | 3.1E+01 | 3.2E+03 |
| IL-21_M36T | 2.27 | 1.1E+02 | 2.8E+01 | 6.7E+01 | 3.1E+03 |
| IL-21_Q41S | 2.47 | 1.4E+02 | 2.6E+01 | 2.5E+01 | 3.0E+03 |
| IL-21_R139S | 2.45 | 8.1E+01 | 2.2E+01 | 2.3E+02 | 3.0E+03 |
| IL-21_I96V | 2.32 | 1.9E+02 | 3.2E+01 | 6.2E+01 | 3.0E+03 |
| IL-21_P133I | 2.3 | 1.8E+02 | 4.4E+01 | 1.1E+02 | 2.9E+03 |
| IL-21_E72T | 2.6 | 6.7E+01 | 2.7E+01 | 5.7E+01 | 2.8E+03 |
| IL-21_P108E | 2.32 | 1.9E+04 | 9.2E+02 | 5.5E+02 | 2.8E+03 |
| IL-21_P62S | 2.47 | 1.0E+02 | 1.9E+01 | 2.7E+01 | 2.8E+03 |
| IL-21_Q32K | 2.6 | 2.7E+01 | 1.2E+01 | 2.8E+01 | 2.7E+03 |
| IL-21_D66R | 2.42 | 2.5E+01 | 1.3E+01 | 1.3E+01 | 2.7E+03 |
| IL-21_V57L | 2.3 | 7.3E+01 | 2.2E+01 | 8.8E+01 | 2.7E+03 |
| IL-21_R34P | 2.54 | 1.4E+03 | 7.6E+02 | 4.2E+02 | 2.7E+03 |
| IL-21_S142I | 2.43 | 6.3E+01 | 2.8E+01 | 4.1E+01 | 2.6E+03 |
| IL-21_S109M | 2.51 | 7.2E+01 | 4.3E+02 | 8.1E+01 | 2.5E+03 |
| IL-21_M39I | 2.28 | 1.3E+02 | 3.0E+01 | 3.5E+01 | 2.5E+03 |
| IL-21_N51V | 2.3 | 4.6E+02 | 6.2E+01 | 2.9E+02 | 2.5E+03 |
| IL-21_E93T | 2.6 | 4.6E+01 | 1.3E+01 | 6.6E+01 | 2.4E+03 |
| IL-21_F60H | 2.64 | 6.2E+01 | 1.5E+01 | 3.5E+01 | 2.4E+03 |
| IL-21_N51G | 2.49 | 1.1E+02 | 3.0E+01 | 4.4E+01 | 2.3E+03 |
| IL-21_V57T | 2.41 | 1.7E+02 | 1.3E+02 | 7.8E+01 | 2.3E+03 |
| IL-21_V57M | 2.3 | 5.7E+01 | 2.2E+01 | 2.5E+01 | 2.3E+03 |
| IL-21_L61H | 2.42 | 1.2E+02 | 2.3E+01 | 2.4E+01 | 2.2E+03 |
| IL-21_LA2Y | 2.35 | 4.3E+01 | 1.1E+01 | 2.1E+01 | 2.1E+03 |
| IL-21_D66M | 2.3 | 5.5E+01 | 1.9E+01 | 2.6E+01 | 2.1E+03 |
| IL-21_R34N | 2.54 | 9.6E+02 | 5.2E+01 | 1.3E+02 | 2.1E+03 |
| IL-21_R34F | 2.47 | 3.9E+02 | 5.2E+01 | 1.1E+02 | 2.1E+03 |
| IL-21_V57Y | 2.43 | 4.6E+01 | 1.6E+01 | 1.7E+01 | 2.1E+03 |
| IL-21_K104G | 2.27 | 4.7E+02 | 7.9E+01 | 1.3E+02 | 1.9E+03 |
| IL-21_H151E | 2.48 | 2.9E+02 | 8.2E+02 | 3.0E+02 | 1.7E+03 |
| IL-21_L120M | 2.29 | 5.2E+01 | 3.6E+01 | 5.6E+01 | 1.7E+03 |
| IL-21_K106S | 2.5 | 7.3E+03 | 6.5E+02 | 1.3E+03 | 1.7E+03 |
| IL-21_E72L | 2.31 | 4.7E+01 | 5.2E+02 | 1.6E+02 | 1.7E+03 |
| IL-21_I43L | 2.31 | 5.8E+01 | 2.0E+01 | 6.1E+01 | 1.5E+03 |
| IL-21_P108D | 2.38 | 1.9E+04 | 5.9E+02 | 2.4E+02 | 1.4E+03 |
| IL-21_V53S | 2.77 | 4.7E+01 | 2.0E+01 | 5.6E+01 | 1.4E+03 |
| IL-21_R34Q | 2.51 | 2.0E+02 | 3.5E+01 | 1.2E+02 | 1.1E+03 |
| IL-21_V53A | 2.38 | 3.2E+01 | 1.7E+01 | 5.6E+01 | 1.1E+03 |
| IL-21_K106N | 2.48 | 1.0E+04 | 5.5E+02 | 6.5E+02 | 1.0E+03 |
| IL-21_A112P | 2.28 | 5.8E+01 | 2.1E+01 | 8.0E+01 | 1.0E+03 |
| IL-21_Q150D | 2.32 | 5.6E+01 | 2.1E+01 | 5.9E+01 | 9.6E+02 |
| IL-21_Q150S | 2.66 | 4.3E+01 | 1.8E+01 | 1.2E+02 | 8.7E+02 |
| IL-21_K106P | 2.46 | 2.9E+02 | 3.0E+01 | 6.8E+01 | 8.7E+02 |
| IL-21_R34A | 2.66 | 3.6E+02 | 3.8E+01 | 2.2E+02 | 8.6E+02 |
| IL-21_N70A | 2.26 | 3.3E+01 | 1.7E+01 | 9.4E+01 | 8.5E+02 |
| IL-21_R34V | 2.44 | 1.4E+02 | 2.7E+01 | 1.1E+02 | 8.4E+02 |
| IL-21_L120Y | 2.4 | 2.8E+01 | 1.3E+01 | 5.8E+01 | 7.9E+02 |
| IL-21_R38F | 2.48 | 4.9E+01 | 2.3E+01 | 2.9E+02 | 7.9E+02 |
| IL-21_S77M | 2.46 | 4.0E+01 | 1.5E+01 | 6.0E+01 | 7.7E+02 |
| IL-21_K85S | 2.42 | 1.5E+02 | 2.2E+01 | 3.2E+01 | 7.1E+02 |
| IL-21_R94S | 2.31 | 1.4E+02 | 2.7E+01 | 3.5E+01 | 5.7E+02 |
| IL-21_H35P | 2.4 | 4.1E+01 | 2.2E+01 | 5.9E+01 | 5.6E+02 |
| IL-21_R114F | 2.47 | 1.4E+02 | 4.8E+01 | 5.7E+01 | 4.5E+02 |
| IL-21_S142D | 2.46 | 3.8E+01 | 1.3E+01 | 6.7E+01 | 4.5E+02 |
| IL-21_R34M | 2.6 | 1.5E+02 | 2.8E+01 | 6.7E+01 | 3.9E+02 |
| IL-21_R34T | 2.58 | 1.8E+02 | 2.6E+01 | 1.4E+02 | 3.5E+02 |
| IL-21_R34L | 2.38 | 1.0E+02 | 2.2E+01 | 7.7E+01 | 3.3E+02 |
| IL-21_R94T | 2.48 | 2.1E+02 | 2.7E+01 | 8.0E+01 | 2.7E+02 |
| IL-21_R115I | 2.3 | 1.4E+02 | 3.5E+01 | 5.0E+01 | 2.6E+02 |
| IL-21_N51E | 2.51 | 2.0E+01 | 1.1E+01 | 1.4E+02 | 2.5E+02 |
| IL-21_K131V | 2.25 | 2.6E+01 | 1.3E+01 | 6.9E+01 | 2.2E+02 |
| IL-21_K106T | 2.32 | 2.0E+02 | 2.2E+02 | 2.9E+02 | 2.1E+02 |
| IL-21_P107D | 2.36 | 6.0E+01 | 2.3E+01 | 3.1E+02 | 1.9E+02 |
| IL-21_H118I | 2.35 | 3.6E+02 | 7.4E+01 | 2.9E+01 | 1.5E+02 |
| IL-21_K106E | 2.4 | 1.9E+04 | 8.5E+02 | 5.5E+02 | 1.2E+02 |
| IL-21_R115Q | 2.41 | 3.2E+01 | 1.1E+01 | 5.6E+01 | 7.4E+01 |
| IL-21_R115P | 2.32 | 4.2E+01 | 1.5E+01 | 5.7E+01 | 5.5E+01 |

Table 2 below discloses the polypeptide sequence of the 273 human IL-21 variants comprising single amino acid substitutions disclosed in Table 1, with the single amino acid substitutions occurring at locations throughout the length of the wild-type human IL-21 polypeptide (SEQ ID NO: 1).

TABLE 2
Polypeptide Sequences of Human IL-21 Variants Comprising Single Amino
Acid Substitutions

| | | SEQ |
|---|---|---|
| | | ID |
| Variant | Amino Acid Sequence | NO |
| IL-21_ | MRSSPGNMERIVICLMVIFLGTLVHKSSSQGQDRHMIRMR | 15 |
| Y52D | QLIDIVDQLKNDVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 16 |
| V46Q | QLIDIQDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 17 |
| W73K | QLIDIVDQLKNYVNDLVPEFLPAPEDVETNCEKSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRKR | 18 |
| M39K | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRDR | 19 |
| M39D | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 20 |
| A75N | QLIDIVDQLKNYVNDLVPEFLPAPEDVETNCEWSNFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 21 |
| V46K | QLIDIKDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 22 |
| L56S | QLIDIVDQLKNYVNDSVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 23 |
| Q80D | QLIDIVDQLKNYVNDLVPEFLPAPEDVETNCEWSAFSCFD | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 24 |
| V46T | QLIDITDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 25 |
| V57H | QLIDIVDQLKNYVNDLHPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 26 |
| F76Q | QLIDIVDQLKNYVNDLVPEFLPAPEDVETNCEWSAQSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRQR | 27 |
| M39Q | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 28 |
| D44P | QLIPIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 29 |
| L103E | QLIDIVDQLKNYVNDLVPEFLPAPEDVETNCEWSAFSCFQ | |
| | KAQLKSANTGNNERIINVSIKKEKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 30 |
| 196D | QLIDIVDQLKNYVNDLVPEFLPAPEDVETNCEWSAFSCFQ | |
| | KAQLKSANTGNNERIDNVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 31 |
| V53N | QLIDIVDQLKNYNNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 32 |
| L42H | QHIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 33 |
| L49E | QLIDIVDQEKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 34 |
| D47P | QLIDIVPQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 35 |
| 1100E | QLIDIVDQLKNYVNDLVPEFLPAPEDVETNCEWSAFSCFQ | |
| | KAQLKSANTGNNERIINVSEKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 36 |
| V46N | QLIDINDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 37 |
| R105Q | QLIDIVDQLKNYVNDLVPEFLPAPEDVETNCEWSAFSCFQ | |
| | KAQLKSANTGNNERIINVSIKKLKQKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 38 |
| V46G | QLIDIGDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 39 |
| F76P | QLIDIVDQLKNYVNDLVPEFLPAPEDVETNCEWSAPSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 40 |
| W73V | QLIDIVDQLKNYVNDLVPEFLPAPEDVETNCEVSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 41 |
| 143P | QLPDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 42 |
| Q80A | QLIDIVDQLKNYVNDLVPEFLPAPEDVETNCEWSAFSCFA | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 43 |
| R105D | QLIDIVDQLKNYVNDLVPEFLPAPEDVETNCEWSAFSCFQ | |
| | KAQLKSANTGNNERIINVSIKKLKDKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 44 |
| F76H | QLIDIVDQLKNYVNDLVPEFLPAPEDVETNCEWSAHSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 45 |
| L42E | QEIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 46 |
| W73T | QLIDIVDQLKNYVNDLVPEFLPAPEDVETNCETSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 47 |
| F79P | QLIDIVDQLKNYVNDLVPEFLPAPEDVETNCEWSAFSCPQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 48 |
| L42G | QGIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 49 |
| 143D | QLDDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 50 |
| P108T | QLIDIVDQLKNYVNDLVPEFLPAPEDVETNCEWSAFSCFQ | |
| | KAQLKSANTGNNERIINVSIKKLKRKPTSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 51 |
| Q41K | KLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 52 |
| L49H | QLIDIVDQHKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 53 |
| F76R | QLIDIVDQLKNYVNDLVPEFLPAPEDVETNCEWSARSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 54 |
| V46E | QLIDIEDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIVMR | 55 |
| R38V | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 56 |
| L49D | QLIDIVDQDKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 57 |
| F76D | QLIDIVDQLKNYVNDLVPEFLPAPEDVETNCEWSADSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 58 |
| W73S | QLIDIVDQLKNYVNDLVPEFLPAPEDVETNCESSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 59 |
| V57P | QLIDIVDQLKNYVNDLPPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRRR | 60 |
| M39R | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 61 |
| Y52K | QLIDIVDQLKNKVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 62 |
| F76T | QLIDIVDQLKNYVNDLVPEFLPAPEDVETNCEWSATSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 63 |
| F79Y | QLIDIVDQLKNYVNDLVPEFLPAPEDVETNCEWSAFSCYQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRER | 64 |
| M39E | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 65 |
| 145G | QLIDGVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 66 |
| D47S | QLIDIVSQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 67 |
| F79K | QLIDIVDQLKNYVNDLVPEFLPAPEDVETNCEWSAFSCKQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 68 |
| L49K | QLIDIVDQKKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 69 |
| L42Q | QQIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 70 |
| 145H | QLIDHVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 71 |
| L49N | QLIDIVDQNKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 72 |
| 145R | QLIDRVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMRRMR | 73 |
| 137R | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 74 |
| L42N | QNIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 75 |
| R105S | QLIDIVDQLKNYVNDLVPEFLPAPEDVETNCEWSAFSCFQ | |
| | KAQLKSANTGNNERIINVSIKKLKSKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 76 |
| 143S | QLSDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 77 |
| L49P | QLIDIVDQPKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 78 |
| 195P | QLIDIVDQLKNYVNDLVPEFLPAPEDVETNCEWSAFSCFQ | |
| | KAQLKSANTGNNERPINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIAMR | 79 |
| R38A | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 80 |
| 145S | QLIDSVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 81 |
| R105W | QLIDIVDQLKNYVNDLVPEFLPAPEDVETNCEWSAFSCFQ | |
| | KAQLKSANTGNNERIINVSIKKLKWKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 82 |
| V46P | QLIDIPDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 83 |
| 145Y | QLIDYVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIQMR | 84 |
| R38Q | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 85 |
| 196P | QLIDIVDQLKNYVNDLVPEFLPAPEDVETNCEWSAFSCFQ | |
| | KAQLKSANTGNNERIPNVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 86 |
| F76E | QLIDIVDQLKNYVNDLVPEFLPAPEDVETNCEWSAESCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 87 |
| R105A | QLIDIVDQLKNYVNDLVPEFLPAPEDVETNCEWSAFSCFQ | |
| | KAQLKSANTGNNERIINVSIKKLKAKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 88 |
| V46D | QLIDIDDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 89 |
| V53P | QLIDIVDQLKNYPNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRPR | 90 |
| M39P | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 91 |
| 195K | QLIDIVDQLKNYVNDLVPEFLPAPEDVETNCEWSAFSCFQ | |
| | KAQLKSANTGNNERKINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 92 |
| L42K | QKIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 93 |
| R105K | QLIDIVDQLKNYVNDLVPEFLPAPEDVETNCEWSAFSCFQ | |
| | KAQLKSANTGNNERIINVSIKKLKKKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIKMR | 94 |
| R38K | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRGR | 95 |
| M39G | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 96 |
| L56F | QLIDIVDQLKNYVNDFVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMSRMR | 97 |
| 137S | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 98 |
| D44A | QLIAIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMNRMR | 99 |
| 137N | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 100 |
| 196T | QLIDIVDQLKNYVNDLVPEFLPAPEDVETNCEWSAFSCFQ | |
| | KAQLKSANTGNNERITNVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 101 |
| L56N | QLIDIVDQLKNYVNDNVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 102 |
| 145P | QLIDPVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 103 |
| 196G | QLIDIVDQLKNYVNDLVPEFLPAPEDVETNCEWSAFSCFQ | |
| | KAQLKSANTGNNERIGNVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 104 |
| E59R | QLIDIVDQLKNYVNDLVPRFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 105 |
| Q41P | PLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 106 |
| 145D | QLIDDVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 107 |
| P108Q | QLIDIVDQLKNYVNDLVPEFLPAPEDVETNCEWSAFSCFQ | |
| | KAQLKSANTGNNERIINVSIKKLKRKPQSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 108 |
| L49S | QLIDIVDQSKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 109 |
| L42D | QDIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 110 |
| L42S | QSIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 111 |
| Q48K | QLIDIVDKLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 112 |
| F79T | QLIDIVDQLKNYVNDLVPEFLPAPEDVETNCEWSAFSCTQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 113 |
| 145N | QLIDNVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 114 |
| W73P | QLIDIVDQLKNYVNDLVPEFLPAPEDVETNCEPSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 115 |
| D44G | QLIGIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 116 |
| L120V | QLIDIVDQLKNYVNDLVPEFLPAPEDVETNCEWSAFSCFQ | |
| | KAQLKSANTGNNERIINVSIKKLKRKPPSTNAGRRQKHRV | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 117 |
| 1148S | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMSHQHLSSRTHGSE | |
| | DS | |
| IL-21_ | MRSSPGNMERIVICLMVIFLGTLVHKSSSQGQDRHMIRMR | 118 |
| F79D | QLIDIVDQLKNYVNDLVPEFLPAPEDVETNCEWSAFSCDQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMGRMR | 119 |
| 137G | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 120 |
| L49R | QLIDIVDQRKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 121 |
| 196A | QLIDIVDQLKNYVNDLVPEFLPAPEDVETNCEWSAFSCFQ | |
| | KAQLKSANTGNNERIANVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 122 |
| 195H | QLIDIVDQLKNYVNDLVPEFLPAPEDVETNCEWSAFSCFQ | |
| | KAQLKSANTGNNERHINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 123 |
| W73G | QLIDIVDQLKNYVNDLVPEFLPAPEDVETNCEGSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 124 |
| V98K | QLIDIVDQLKNYVNDLVPEFLPAPEDVETNCEWSAFSCFQ | |
| | KAQLKSANTGNNERIINKSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 125 |
| V53Q | QLIDIVDQLKNYQNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 126 |
| P108G | QLIDIVDQLKNYVNDLVPEFLPAPEDVETNCEWSAFSCFQ | |
| | KAQLKSANTGNNERIINVSIKKLKRKPGSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQPRHMIRMR | 127 |
| D33P | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 128 |
| F79R | QLIDIVDQLKNYVNDLVPEFLPAPEDVETNCEWSAFSCRQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 129 |
| W73N | QLIDIVDQLKNYVNDLVPEFLPAPEDVETNCENSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 130 |
| 196S | QLIDIVDQLKNYVNDLVPEFLPAPEDVETNCEWSAFSCFQ | |
| | KAQLKSANTGNNERISNVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIMMR | 131 |
| R38M | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 132 |
| V98R | QLIDIVDQLKNYVNDLVPEFLPAPEDVETNCEWSAFSCFQ | |
| | KAQLKSANTGNNERIINRSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 133 |
| P132A | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKAPKEFLERFKSLLQKMIHQHLSSRTHGSE | |
| | DS | |
| IL-21_ | MRSSPGNMERIVICLMVIFLGTLVHKSSSQGQDRHMIRMR | 134 |
| L42R | QRIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQRRHMIRMR | 135 |
| D33R | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 136 |
| Y52N | QLIDIVDQLKNNVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 137 |
| L56E | QLIDIVDQLKNYVNDEVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMERMR | 138 |
| 137E | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIEMR | 139 |
| R38E | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 140 |
| F79E | QLIDIVDQLKNYVNDLVPEFLPAPEDVETNCEWSAFSCEQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 141 |
| P62V | QLIDIVDQLKNYVNDLVPEFLVAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 142 |
| L56T | QLIDIVDQLKNYVNDTVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMKRMR | 143 |
| 137K | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 144 |
| S74H | QLIDIVDQLKNYVNDLVPEFLPAPEDVETNCEWHAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 145 |
| Y52H | QLIDIVDQLKNHVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 146 |
| D44T | QLITIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 147 |
| Y52Q | QLIDIVDQLKNQVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 148 |
| D44N | QLINIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 149 |
| S99H | QLIDIVDQLKNYVNDLVPEFLPAPEDVETNCEWSAFSCFQ | |
| | KAQLKSANTGNNERIINVHIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 150 |
| V53H | QLIDIVDQLKNYHNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 151 |
| S77R | QLIDIVDQLKNYVNDLVPEFLPAPEDVETNCEWSAFRCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 152 |
| S109K | QLIDIVDQLKNYVNDLVPEFLPAPEDVETNCEWSAFSCFQ | |
| | KAQLKSANTGNNERIINVSIKKLKRKPPKTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 153 |
| 195T | QLIDIVDQLKNYVNDLVPEFLPAPEDVETNCEWSAFSCFQ | |
| | KAQLKSANTGNNERTINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 154 |
| W73L | QLIDIVDQLKNYVNDLVPEFLPAPEDVETNCELSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 155 |
| S74N | QLIDIVDQLKNYVNDLVPEFLPAPEDVETNCEWNAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 156 |
| Q145H | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLHKMIHQHLSSRTHGSE | |
| | DS | |
| IL-21_ | MRSSPGNMERIVICLMVIFLGTLVHKSSSQGQDRHMIRMR | 157 |
| V53E | QLIDIVDQLKNYENDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 158 |
| V53T | QLIDIVDQLKNYTNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 159 |
| K131M | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKMPPKEFLERFKSLLQKMIHQHLSSRTHGSE | |
| | DS | |
| IL-21_ | MRSSPGNMERIVICLMVIFLGTLVHKSSSQGQDRHMIRMR | 160 |
| W73A | QLIDIVDQLKNYVNDLVPEFLPAPEDVETNCEASAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDSHMIRMR | 161 |
| R34S | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 162 |
| L120H | QLIDIVDQLKNYVNDLVPEFLPAPEDVETNCEWSAFSCFQ | |
| | KAQLKSANTGNNERIINVSIKKLKRKPPSTNAGRRQKHRH | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 163 |
| N88K | QLIDIVDQLKNYVNDLVPEFLPAPEDVETNCEWSAFSCFQ | |
| | KAQLKSAKTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDWHMIRMR | 164 |
| R34W | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQARHMIRMR | 165 |
| D33A | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 166 |
| L611 | QLIDIVDQLKNYVNDLVPEFIPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMQRMR | 167 |
| 137Q | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDDHMIRMR | 168 |
| R34D | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 169 |
| T89L | QLIDIVDQLKNYVNDLVPEFLPAPEDVETNCEWSAFSCFQ | |
| | KAQLKSANLGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQTRHMIRMR | 170 |
| D33T | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 171 |
| W73Q | QLIDIVDQLKNYVNDLVPEFLPAPEDVETNCEQSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 172 |
| S77K | QLIDIVDQLKNYVNDLVPEFLPAPEDVETNCEWSAFKCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDKHMIRMR | 173 |
| R34K | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQNRHMIRMR | 174 |
| D33N | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 175 |
| V57R | QLIDIVDQLKNYVNDLRPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 176 |
| Q150R | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHRHLSSRTHGSE | |
| | DS | |
| IL-21_ | MRSSPGNMERIVICLMVIFLGTLVHKSSSQGQSRHMIRMR | 177 |
| D33S | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 178 |
| E72S | QLIDIVDQLKNYVNDLVPEFLPAPEDVETNCSWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 179 |
| H118R | QLIDIVDQLKNYVNDLVPEFLPAPEDVETNCEWSAFSCFQ | |
| | KAQLKSANTGNNERIINVSIKKLKRKPPSTNAGRRQKRRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 180 |
| S142H | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKHLLQKMIHQHLSSRTHGSE | |
| | DS | |
| IL-21_ | MRSSPGNMERIVICLMVIFLGTLVHKSSSQGQDHHMIRMR | 181 |
| R34H | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDGHMIRMR | 182 |
| R34G | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 183 |
| V98H | QLIDIVDQLKNYVNDLVPEFLPAPEDVETNCEWSAFSCFQ | |
| | KAQLKSANTGNNERIINHSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 184 |
| L120K | QLIDIVDQLKNYVNDLVPEFLPAPEDVETNCEWSAFSCFQ | |
| | KAQLKSANTGNNERIINVSIKKLKRKPPSTNAGRRQKHRK | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 185 |
| N51P | QLIDIVDQLKPYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 186 |
| 145A | QLIDAVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 187 |
| P108L | QLIDIVDQLKNYVNDLVPEFLPAPEDVETNCEWSAFSCFQ | |
| | KAQLKSANTGNNERIINVSIKKLKRKPLSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 188 |
| N54K | QLIDIVDQLKNYVKDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 189 |
| A112R | QLIDIVDQLKNYVNDLVPEFLPAPEDVETNCEWSAFSCFQ | |
| | KAQLKSANTGNNERIINVSIKKLKRKPPSTNRGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 190 |
| F601 | QLIDIVDQLKNYVNDLVPEILPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 191 |
| N97G | QLIDIVDQLKNYVNDLVPEFLPAPEDVETNCEWSAFSCFQ | |
| | KAQLKSANTGNNERIIGVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQQRHMIRMR | 192 |
| D33Q | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 193 |
| E93G | QLIDIVDQLKNYVNDLVPEFLPAPEDVETNCEWSAFSCFQ | |
| | KAQLKSANTGNNGRIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 194 |
| H118K | QLIDIVDQLKNYVNDLVPEFLPAPEDVETNCEWSAFSCFQ | |
| | KAQLKSANTGNNERIINVSIKKLKRKPPSTNAGRRQKKRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 195 |
| L56M | QLIDIVDQLKNYVNDMVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 196 |
| P107Y | QLIDIVDQLKNYVNDLVPEFLPAPEDVETNCEWSAFSCFQ | |
| | KAQLKSANTGNNERIINVSIKKLKRKYPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 197 |
| E68R | QLIDIVDQLKNYVNDLVPEFLPAPEDVRTNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 198 |
| S109R | QLIDIVDQLKNYVNDLVPEFLPAPEDVETNCEWSAFSCFQ | |
| | KAQLKSANTGNNERIINVSIKKLKRKPPRTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 199 |
| N54P | QLIDIVDQLKNYVPDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 200 |
| P62H | QLIDIVDQLKNYVNDLVPEFLHAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 201 |
| E59S | QLIDIVDQLKNYVNDLVPSFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 202 |
| E721 | QLIDIVDQLKNYVNDLVPEFLPAPEDVETNCIWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 203 |
| P62T | QLIDIVDQLKNYVNDLVPEFLTAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 204 |
| L152H | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHHSSRTHGSE | |
| | DS | |
| IL-21_ | MRSSPGNMERIVICLMVIFLGTLVHKSSSQGQDRHMIRMR | 205 |
| S74A | QLIDIVDQLKNYVNDLVPEFLPAPEDVETNCEWAAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 206 |
| N70H | QLIDIVDQLKNYVNDLVPEFLPAPEDVETHCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 207 |
| F76M | QLIDIVDQLKNYVNDLVPEFLPAPEDVETNCEWSAMSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 208 |
| S861 | QLIDIVDQLKNYVNDLVPEFLPAPEDVETNCEWSAFSCFQ | |
| | KAQLKIANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 209 |
| F60R | QLIDIVDQLKNYVNDLVPERLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 210 |
| P1081 | QLIDIVDQLKNYVNDLVPEFLPAPEDVETNCEWSAFSCFQ | |
| | KAQLKSANTGNNERIINVSIKKLKRKPISTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 211 |
| N54H | QLIDIVDQLKNYVHDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDEHMIRMR | 212 |
| R34E | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 213 |
| A87N | QLIDIVDQLKNYVNDLVPEFLPAPEDVETNCEWSAFSCFQ | |
| | KAQLKSNNTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 214 |
| W73Y | QLIDIVDQLKNYVNDLVPEFLPAPEDVETNCEYSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 215 |
| 143M | QLMDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 216 |
| E72H | QLIDIVDQLKNYVNDLVPEFLPAPEDVETNCHWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDYHMIRMR | 217 |
| R34Y | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 218 |
| A63T | QLIDIVDQLKNYVNDLVPEFLPTPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 219 |
| E68K | QLIDIVDQLKNYVNDLVPEFLPAPEDVKTNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 220 |
| Q48G | QLIDIVDGLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMVRMR | 221 |
| 137V | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHTIRMR | 222 |
| M36T | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 223 |
| Q41S | SLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 224 |
| R139S | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLESFKSLLQKMIHQHLSSRTHGSE | |
| | DS | |
| IL-21_ | MRSSPGNMERIVICLMVIFLGTLVHKSSSQGQDRHMIRMR | 225 |
| 196V | QLIDIVDQLKNYVNDLVPEFLPAPEDVETNCEWSAFSCFQ | |
| | KAQLKSANTGNNERIVNVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 226 |
| P1331 | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPIKEFLERFKSLLQKMIHQHLSSRTHGSE | |
| | DS | |
| IL-21_ | MRSSPGNMERIVICLMVIFLGTLVHKSSSQGQDRHMIRMR | 227 |
| E72T | QLIDIVDQLKNYVNDLVPEFLPAPEDVETNCTWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 228 |
| P108E | QLIDIVDQLKNYVNDLVPEFLPAPEDVETNCEWSAFSCFQ | |
| | KAQLKSANTGNNERIINVSIKKLKRKPESTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 229 |
| P62S | QLIDIVDQLKNYVNDLVPEFLSAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGKDRHMIRMR | 230 |
| Q32K | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 231 |
| D66R | QLIDIVDQLKNYVNDLVPEFLPAPERVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 232 |
| V57L | QLIDIVDQLKNYVNDLLPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDPHMIRMR | 233 |
| R34P | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 234 |
| S1421 | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKILLQKMIHQHLSSRTHGSE | |
| | DS | |
| IL-21_ | MRSSPGNMERIVICLMVIFLGTLVHKSSSQGQDRHMIRMR | 235 |
| S109M | QLIDIVDQLKNYVNDLVPEFLPAPEDVETNCEWSAFSCFQ | |
| | KAQLKSANTGNNERIINVSIKKLKRKPPMTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRIR | 236 |
| M391 | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 237 |
| N51V | QLIDIVDQLKVYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 238 |
| E93T | QLIDIVDQLKNYVNDLVPEFLPAPEDVETNCEWSAFSCFQ | |
| | KAQLKSANTGNNTRIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 239 |
| F60H | QLIDIVDQLKNYVNDLVPEHLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 240 |
| N51G | QLIDIVDQLKGYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 241 |
| V57T | QLIDIVDQLKNYVNDLTPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 242 |
| V57M | QLIDIVDQLKNYVNDLMPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 243 |
| L61H | QLIDIVDQLKNYVNDLVPEFHPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 244 |
| L42Y | QYIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 245 |
| D66M | QLIDIVDQLKNYVNDLVPEFLPAPEMVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDNHMIRMR | 246 |
| R34N | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDFHMIRMR | 247 |
| R34F | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 248 |
| V57Y | QLIDIVDQLKNYVNDLYPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 249 |
| K104G | QLIDIVDQLKNYVNDLVPEFLPAPEDVETNCEWSAFSCFQ | |
| | KAQLKSANTGNNERIINVSIKKLGRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 250 |
| H151E | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQELSSRTHGSE | |
| | DS | |
| IL-21_ | MRSSPGNMERIVICLMVIFLGTLVHKSSSQGQDRHMIRMR | 251 |
| L120M | QLIDIVDQLKNYVNDLVPEFLPAPEDVETNCEWSAFSCFQ | |
| | KAQLKSANTGNNERIINVSIKKLKRKPPSTNAGRRQKHRM | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 252 |
| K106S | QLIDIVDQLKNYVNDLVPEFLPAPEDVETNCEWSAFSCFQ | |
| | KAQLKSANTGNNERIINVSIKKLKRSPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 253 |
| E72L | QLIDIVDQLKNYVNDLVPEFLPAPEDVETNCLWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 254 |
| 143L | QLLDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 255 |
| P108D | QLIDIVDQLKNYVNDLVPEFLPAPEDVETNCEWSAFSCFQ | |
| | KAQLKSANTGNNERIINVSIKKLKRKPDSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 256 |
| V53S | QLIDIVDQLKNYSNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDQHMIRMR | 257 |
| R34Q | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 258 |
| V53A | QLIDIVDQLKNYANDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 259 |
| K106N | QLIDIVDQLKNYVNDLVPEFLPAPEDVETNCEWSAFSCFQ | |
| | KAQLKSANTGNNERIINVSIKKLKRNPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 260 |
| A112P | QLIDIVDQLKNYVNDLVPEFLPAPEDVETNCEWSAFSCFQ | |
| | KAQLKSANTGNNERIINVSIKKLKRKPPSTNPGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 261 |
| Q150D | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHDHLSSRTHGSE | |
| | DS | |
| IL-21_ | MRSSPGNMERIVICLMVIFLGTLVHKSSSQGQDRHMIRMR | 262 |
| Q150S | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHSHLSSRTHGSE | |
| | DS | |
| IL-21_ | MRSSPGNMERIVICLMVIFLGTLVHKSSSQGQDRHMIRMR | 263 |
| K106P | QLIDIVDQLKNYVNDLVPEFLPAPEDVETNCEWSAFSCFQ | |
| | KAQLKSANTGNNERIINVSIKKLKRPPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDAHMIRMR | 264 |
| R34A | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 265 |
| N70A | QLIDIVDQLKNYVNDLVPEFLPAPEDVETACEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDVHMIRMR | 266 |
| R34V | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 267 |
| L120Y | QLIDIVDQLKNYVNDLVPEFLPAPEDVETNCEWSAFSCFQ | |
| | KAQLKSANTGNNERIINVSIKKLKRKPPSTNAGRRQKHRY | |
| | TCPSCDSYEKKPPKEFLEREKSLLQKMIHQHLSSRTHGSE | |
| | DS | |
| IL-21_ | MRSSPGNMERIVICLMVIFLGTLVHKSSSQGQDRHMIFMR | 268 |
| R38F | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 269 |
| S77M | QLIDIVDQLKNYVNDLVPEFLPAPEDVETNCEWSAFMCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 270 |
| K85S | QLIDIVDQLKNYVNDLVPEFLPAPEDVETNCEWSAFSCFQ | |
| | KAQLSSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 271 |
| R94S | QLIDIVDQLKNYVNDLVPEFLPAPEDVETNCEWSAFSCFQ | |
| | KAQLKSANTGNNESIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRPMIRMR | 272 |
| H35P | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 273 |
| R114F | QLIDIVDQLKNYVNDLVPEFLPAPEDVETNCEWSAFSCFQ | |
| | KAQLKSANTGNNERIINVSIKKLKRKPPSTNAGERQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 274 |
| S142D | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKDLLQKMIHQHLSSRTHGSE | |
| | DS | |
| IL-21_ | MRSSPGNMERIVICLMVIFLGTLVHKSSSQGQDMHMIRMR | 275 |
| R34M | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDTHMIRMR | 276 |
| R34T | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDLHMIRMR | 277 |
| R34L | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 278 |
| R94T | QLIDIVDQLKNYVNDLVPEFLPAPEDVETNCEWSAFSCFQ | |
| | KAQLKSANTGNNETIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 279 |
| R1151 | QLIDIVDQLKNYVNDLVPEFLPAPEDVETNCEWSAFSCFQ | |
| | KAQLKSANTGNNERIINVSIKKLKRKPPSTNAGRIQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 280 |
| N51E | QLIDIVDQLKEYVNDLVPEFLPAPEDVETNCEWSAFSCFQ | |
| | KAQLKSANTGNNERIINVSIKKLKRKPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 281 |
| K131V | QLIDIVDQLKNYVNDLVPEFLPAPEDVETNCEWSAFSCFQ | |
| | KAQLKSANTGNNERIINVSIKKLKRKPPSTNAGRRQKHRL | |
| | TCPSCDSYEKVPPKEFLERFKSLLQKMIHQHLSSRTHGSE | |
| | DS | |
| IL-21_ | MRSSPGNMERIVICLMVIFLGTLVHKSSSQGQDRHMIRMR | 282 |
| K106T | QLIDIVDQLKNYVNDLVPEFLPAPEDVETNCEWSAFSCFQ | |
| | KAQLKSANTGNNERIINVSIKKLKRTPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 283 |
| P107D | QLIDIVDQLKNYVNDLVPEFLPAPEDVETNCEWSAFSCFQ | |
| | KAQLKSANTGNNERIINVSIKKLKRKDPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 284 |
| H1181 | QLIDIVDQLKNYVNDLVPEFLPAPEDVETNCEWSAFSCFQ | |
| | KAQLKSANTGNNERIINVSIKKLKRKPPSTNAGRRQKIRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 285 |
| K106E | QLIDIVDQLKNYVNDLVPEFLPAPEDVETNCEWSAFSCFQ | |
| | KAQLKSANTGNNERIINVSIKKLKREPPSTNAGRRQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 286 |
| R115Q | QLIDIVDQLKNYVNDLVPEFLPAPEDVETNCEWSAFSCFQ | |
| | KAQLKSANTGNNERIINVSIKKLKRKPPSTNAGRQQKHRL | |
| | TCPSCDSYEKKPPKEFLERFKSLLQKMIHQHLSSRTHGSE | |
| | DS | |
| IL-21_ | MRSSPGNMERIVICLMVIFLGTLVHKSSSQGQDRHMIRMR | 287 |
| R115P | QLIDIVDQLKNYVNDLVPEFLPAPEDVETNCEWSAFSCFQ | |
| | KAQLKSANTGNNERIINVSIKKLKRKPPSTNAGRPQKHRL | |
| | TCPSCDSYEKKPPKEFLERFKSLLQKMIHQHLSSRTHGSE | |
| | DS | |

Table 3 below discloses 812 mouse IL-21 variants comprising single amino acid substitutions, with single amino acid substitutions throughout the length of the wild-type mouse IL-21 polypeptide (SEQ ID NO: 3) with reduced affinity for mouse IL-21R (SEQ ID NO: 4). Any variant found to bind mouse IL-21R with a half-log affinity weaker than wild-type mouse IL-21 as measured by AlphaSeq™ (cutoff=2.5 μM in unbuffered media, 10 nM in pH 7 media, 23 nM in pH 6 media, and 101 nM in pH 5 media) and found to have an average expression bin>2.0 is included in Table 3. Table 3 is sorted in order of descending K_{d} in the unbuffered condition, with the highest values representing the lowest affinity of mouse IL-21 for mouse IL-21R.

TABLE 3
Mouse IL-21 Variants Comprising Single Amino Acid Substitutions

| | Average | Affinity, | Affinity, | Affinity, | Affinity, |
|---|---|---|---|---|---|
| | expression | pH 5 | pH 6 | pH 7 | unbuffered |
| Variant | bin | (nM) | (nM) | (nM) | (nM) |
| IL-21m_Y121L | 2.57 | 1.0E+04 | 3.8E+02 | 5.2E+01 | 5.5E+05 |
| IL-21m_L136G | 2.47 | 1.1E+05 | NA | 2.5E+02 | 4.4E+05 |
| IL-21m_L130G | 2.34 | 1.2E+05 | 6.5E+04 | NA | 3.9E+05 |
| IL-21m_P101W | 2.05 | 5.7E+04 | 5.6E+03 | 3.2E+02 | 3.5E+05 |
| IL-21m_L35G | 2.49 | 1.3E+05 | 5.8E+03 | 4.7E+02 | 3.4E+05 |
| IL-21m_M140E | 2.08 | 1.3E+04 | 1.7E+02 | 6.2E+01 | 3.3E+05 |
| IL-21m_L130K | 2.03 | 9.0E+04 | NA | NA | 3.3E+05 |
| IL-21m_Q95V | 2.02 | 4.7E+04 | 1.2E+02 | 3.5E+01 | 3.2E+05 |
| IL-21m_N85F | 2.56 | 2.8E+04 | 1.2E+03 | 1.5E+02 | 3.1E+05 |
| IL-21m_R98V | 2.19 | 3.6E+02 | 2.3E+01 | 4.1E+01 | 3.1E+05 |
| IL-21m_I38P | 2.72 | 1.3E+05 | 5.0E+04 | NA | 3.0E+05 |
| IL-21m_L137W | 2.47 | 1.1E+05 | 4.9E+03 | 8.9E+02 | 3.0E+05 |
| IL-21m_P101Y | 2.32 | 7.8E+04 | 9.5E+02 | 1.0E+02 | 2.9E+05 |
| IL-21m_F72G | 2.56 | 1.5E+05 | 8.0E+03 | 5.5E+02 | 2.9E+05 |
| IL-21m_LA2T | 2.44 | 5.7E+04 | 2.7E+03 | 2.7E+02 | 2.9E+05 |
| IL-21m_F72N | 2.61 | 7.3E+04 | 3.2E+04 | 6.1E+03 | 2.7E+05 |
| IL-21m_F69H | 2.36 | 4.1E+04 | 5.3E+03 | 3.5E+02 | 2.6E+05 |
| IL-21m_E40P | 2.39 | 2.8E+05 | 2.3E+04 | NA | 2.6E+05 |
| IL-21m_L100S | 2.46 | 1.0E+05 | 3.5E+03 | 2.7E+02 | 2.6E+05 |
| IL-21m_I38E | 2.51 | 1.0E+05 | 2.3E+05 | 5.3E+04 | 2.6E+05 |
| IL-21m_L92K | 2.24 | 3.2E+04 | 1.6E+02 | 5.9E+01 | 2.6E+05 |
| IL-21m_L92N | 2.41 | 7.8E+04 | 3.9E+02 | 6.9E+01 | 2.6E+05 |
| IL-21m_L96T | 2.11 | 5.3E+04 | 5.5E+02 | 9.1E+01 | 2.5E+05 |
| IL-21m_A68N | 2.52 | 8.9E+04 | 6.4E+02 | 1.2E+02 | 2.5E+05 |
| IL-21m_I30P | 2.23 | 6.6E+04 | 1.1E+02 | 4.0E+01 | 2.5E+05 |
| IL-21m_P101T | 2.12 | 6.6E+04 | 5.0E+02 | 9.9E+01 | 2.5E+05 |
| IL-21m_Q73D | 2.21 | 3.7E+04 | 1.1E+04 | NA | 2.5E+05 |
| IL-21m_F69N | 2.48 | 1.1E+05 | 9.1E+03 | 3.0E+03 | 2.4E+05 |
| IL-21m_P101S | 2.12 | 9.4E+04 | 1.2E+03 | 1.0E+02 | 2.4E+05 |
| IL-21m_L137D | 2.15 | 7.0E+04 | 5.1E+04 | NA | 2.3E+05 |
| IL-21m_Q73G | 2.43 | 7.2E+03 | 5.2E+01 | 2.1E+01 | 2.3E+05 |
| IL-21m_F69R | 2.28 | 1.3E+05 | 5.0E+03 | 2.8E+03 | 2.3E+05 |
| IL-21m_F72W | 2.03 | 1.2E+05 | 3.3E+03 | 3.5E+02 | 2.3E+05 |
| IL-21m_L96N | 2.35 | 1.1E+05 | 4.5E+04 | NA | 2.3E+05 |
| IL-21m_L92Q | 2.58 | 8.7E+03 | 5.0E+01 | 2.9E+01 | 2.3E+05 |
| IL-21m_L137E | 2.18 | 3.0E+04 | 3.5E+03 | 6.8E+02 | 2.3E+05 |
| IL-21m_P101Q | 2.29 | 6.3E+04 | 1.1E+03 | 1.5E+02 | 2.2E+05 |
| IL-21m_F72P | 2.59 | 1.9E+05 | NA | 1.1E+04 | 2.2E+05 |
| IL-21m_I38W | 2.05 | 8.1E+04 | 1.1E+03 | 1.3E+02 | 2.2E+05 |
| IL-21m_I89W | 2.37 | 1.2E+05 | 2.7E+03 | 2.9E+02 | 2.2E+05 |
| IL-21m_LA2R | 2.38 | 1.8E+05 | 2.6E+04 | NA | 2.2E+05 |
| IL-21m_P101F | 2.09 | 8.8E+04 | 6.9E+03 | 5.7E+02 | 2.1E+05 |
| IL-21m_E122W | 2.36 | 2.6E+04 | 2.3E+02 | 6.5E+01 | 2.1E+05 |
| IL-21m_F129G | 2.29 | 5.3E+04 | 4.4E+02 | 5.9E+01 | 2.1E+05 |
| IL-21m_L32H | 2.69 | 9.6E+04 | 1.9E+04 | 8.6E+03 | 2.1E+05 |
| IL-21m_L100Q | 2.27 | 3.0E+05 | 1.2E+03 | 1.6E+02 | 2.0E+05 |
| IL-21m_L100H | 2.33 | 9.3E+04 | 3.1E+03 | 1.4E+02 | 2.0E+05 |
| IL-21m_V39Y | 2.03 | 8.8E+04 | 1.8E+03 | 2.1E+02 | 2.0E+05 |
| IL-21m_L77Y | 2.32 | 9.1E+04 | 1.5E+03 | 1.7E+02 | 2.0E+05 |
| IL-21m_P101K | 2.08 | 1.2E+05 | 9.2E+03 | 2.2E+02 | 2.0E+05 |
| IL-21m_LA9S | 2.35 | 8.6E+04 | 3.1E+04 | 8.2E+03 | 2.0E+05 |
| IL-21m_L35T | 2.5 | 9.6E+04 | 2.9E+02 | 8.2E+01 | 2.0E+05 |
| IL-21m_L42K | 2.41 | 1.9E+05 | 8.6E+04 | NA | 2.0E+05 |
| IL-21m_L136E | 2.41 | 1.5E+04 | 1.6E+02 | 4.9E+01 | 2.0E+05 |
| IL-21m_L54R | 2.21 | 1.0E+03 | 3.5E+01 | 1.7E+01 | 2.0E+05 |
| IL-21m_R98S | 2.4 | 2.4E+04 | 3.8E+01 | 5.0E+01 | 2.0E+05 |
| IL-21m_P79R | 2.35 | 1.1E+05 | 6.0E+02 | 9.4E+01 | 2.0E+05 |
| IL-21m_Q95R | 2.22 | 2.4E+04 | 2.9E+02 | 9.3E+01 | 2.0E+05 |
| IL-21m_F69D | 2.58 | 1.4E+05 | 1.1E+04 | 2.2E+03 | 2.0E+05 |
| IL-21m_L77Q | 2.44 | 7.9E+04 | 1.2E+04 | 3.3E+03 | 2.0E+05 |
| IL-21m_L77T | 2.31 | 1.2E+05 | 6.3E+04 | 2.5E+03 | 2.0E+05 |
| IL-21m_P57D | 2.39 | 6.3E+04 | 1.6E+04 | 1.7E+03 | 2.0E+05 |
| IL-21m_L42Q | 2.49 | 9.5E+04 | 1.4E+05 | NA | 1.9E+05 |
| IL-21m_P101M | 2.19 | 1.2E+05 | 1.8E+03 | 1.8E+02 | 1.9E+05 |
| IL-21m_P116W | 2.14 | 9.8E+03 | 7.9E+01 | 4.7E+01 | 1.9E+05 |
| IL-21m_L96G | 2.34 | 1.5E+05 | NA | 4.0E+03 | 1.9E+05 |
| IL-21m_L96D | 2.12 | 5.7E+04 | NA | NA | 1.9E+05 |
| IL-21m_F129Q | 2.15 | 3.1E+04 | 4.1E+03 | 5.5E+02 | 1.9E+05 |
| IL-21m_LA9R | 2.08 | 5.8E+04 | 2.2E+04 | NA | 1.9E+05 |
| IL-21m_A94P | 2.23 | 9.4E+04 | 8.4E+03 | 2.8E+02 | 1.9E+05 |
| IL-21m_A68D | 2.34 | 5.3E+04 | 6.4E+03 | 5.8E+02 | 1.9E+05 |
| IL-21m_L100Y | 2.06 | 9.4E+04 | 5.7E+02 | 1.3E+02 | 1.9E+05 |
| IL-21m_V39D | 2.46 | 1.2E+05 | 5.7E+04 | NA | 1.9E+05 |
| IL-21m_L54G | 2.36 | 1.5E+05 | 8.0E+02 | 1.3E+02 | 1.9E+05 |
| IL-21m_H34P | 2.29 | 1.0E+05 | 4.0E+04 | NA | 1.9E+05 |
| IL-21m_D37P | 2.46 | 9.1E+04 | 3.2E+02 | 8.8E+01 | 1.8E+05 |
| IL-21m_D26T | 2 | 7.9E+03 | 4.5E+01 | 2.0E+01 | 1.8E+05 |
| IL-21m_A68R | 2.32 | 6.3E+04 | 3.1E+03 | 8.0E+02 | 1.8E+05 |
| IL-21m_L100N | 2.33 | 1.1E+05 | 9.6E+02 | 1.2E+02 | 1.8E+05 |
| IL-21m_LA2Y | 2.27 | 1.4E+05 | 4.2E+04 | 1.2E+04 | 1.8E+05 |
| IL-21m_A70P | 2.33 | 6.7E+04 | 2.2E+03 | 1.6E+02 | 1.8E+05 |
| IL-21m_L77N | 2.25 | 6.5E+04 | 8.1E+03 | 2.8E+03 | 1.8E+05 |
| IL-21m_F129T | 2.18 | 5.9E+03 | 3.8E+01 | 2.3E+01 | 1.8E+05 |
| IL-21m_L35D | 2.68 | 8.8E+03 | 1.3E+02 | 8.0E+01 | 1.8E+05 |
| IL-21m_P101E | 2.15 | 6.1E+04 | 6.6E+02 | 2.2E+02 | 1.8E+05 |
| IL-21m_E65P | 2.53 | 6.6E+04 | 2.2E+03 | 1.1E+02 | 1.8E+05 |
| IL-21m_F72R | 2.48 | 5.3E+04 | 6.2E+03 | 2.7E+03 | 1.8E+05 |
| IL-21m_L100W | 2.26 | 5.4E+04 | 1.7E+02 | 5.8E+01 | 1.8E+05 |
| IL-21m_LA9K | 2.43 | 9.1E+04 | 3.4E+04 | NA | 1.8E+05 |
| IL-21m_Q138P | 2.01 | 4.7E+04 | 1.1E+03 | 1.9E+02 | 1.8E+05 |
| IL-21m_K74P | 2.2 | 1.2E+05 | 2.3E+03 | 1.7E+02 | 1.8E+05 |
| IL-21m_I141D | 2.2 | 8.6E+04 | 5.8E+02 | 1.0E+02 | 1.8E+05 |
| IL-21m_L35K | 2.55 | 1.1E+05 | NA | 3.6E+02 | 1.8E+05 |
| IL-21m_R98E | 2.15 | 1.9E+04 | 3.9E+02 | 5.0E+02 | 1.8E+05 |
| IL-21m_F69G | 2.58 | 3.5E+04 | 7.1E+03 | NA | 1.8E+05 |
| IL-21m_LA9P | 2.22 | 8.6E+04 | 5.4E+04 | 1.9E+04 | 1.8E+05 |
| IL-21m_F69P | 2.57 | 2.4E+05 | 2.0E+04 | 2.3E+04 | 1.8E+05 |
| IL-21m_E46P | 2.11 | 3.3E+04 | 2.2E+02 | 6.6E+01 | 1.7E+05 |
| IL-21m_I38Y | 2.29 | 1.8E+05 | 2.3E+03 | 2.3E+02 | 1.7E+05 |
| IL-21m_L137Y | 2.34 | 2.6E+04 | 2.4E+03 | 7.5E+02 | 1.7E+05 |
| IL-21m_V93H | 2.24 | 6.7E+04 | 4.1E+02 | 4.1E+01 | 1.7E+05 |
| IL-21m_L96A | 2.12 | 8.2E+04 | 4.2E+03 | 2.7E+02 | 1.7E+05 |
| IL-21m_LA2H | 2.49 | 8.9E+04 | 9.2E+03 | 2.4E+03 | 1.7E+05 |
| IL-21m_K139P | 2.04 | 6.2E+04 | 3.8E+03 | 6.1E+02 | 1.7E+05 |
| IL-21m_V60Q | 2.15 | 2.6E+04 | 2.9E+02 | 6.6E+01 | 1.7E+05 |
| IL-21m_E128N | 2.4 | 4.8E+04 | 7.6E+02 | 1.0E+02 | 1.7E+05 |
| IL-21m_P57H | 2.07 | 6.6E+04 | 6.0E+03 | 1.6E+03 | 1.7E+05 |
| IL-21m_E128P | 2.11 | 7.2E+04 | 6.7E+03 | 3.2E+02 | 1.7E+05 |
| IL-21m_A56G | 2.11 | 2.2E+04 | 1.3E+02 | 3.7E+01 | 1.7E+05 |
| IL-21m_F129D | 2.3 | 5.2E+04 | NA | 9.2E+03 | 1.7E+05 |
| IL-21m_N85L | 2.38 | 7.6E+04 | 4.4E+03 | 4.6E+02 | 1.7E+05 |
| IL-21m_F69T | 2.35 | 2.7E+05 | NA | NA | 1.7E+05 |
| IL-21m_L136P | 2.19 | 4.5E+04 | 1.0E+04 | 2.6E+03 | 1.7E+05 |
| IL-21m_LA2S | 2.39 | 1.5E+05 | 5.0E+04 | 1.1E+04 | 1.7E+05 |
| IL-21m_A68P | 2.35 | 9.1E+04 | 4.8E+03 | 1.6E+03 | 1.7E+05 |
| IL-21m_L100T | 2.34 | 2.4E+04 | 8.8E+01 | 4.7E+01 | 1.7E+05 |
| IL-21m_I30S | 2.09 | 1.4E+05 | 1.7E+02 | 4.2E+01 | 1.7E+05 |
| IL-21m_L137G | 2.27 | 6.8E+04 | 1.5E+03 | 3.0E+02 | 1.7E+05 |
| IL-21m_P101G | 2.16 | 9.8E+04 | 1.5E+03 | 3.0E+02 | 1.7E+05 |
| IL-21m_A56P | 2.09 | 2.3E+04 | 2.2E+02 | 4.8E+01 | 1.7E+05 |
| IL-21m_Q73F | 2.2 | 7.0E+04 | 2.1E+03 | 3.4E+02 | 1.7E+05 |
| IL-21m_L137F | 2.18 | 4.6E+04 | 5.8E+02 | 9.7E+01 | 1.7E+05 |
| IL-21m_L32D | 2.87 | 9.1E+04 | 6.0E+03 | 3.2E+03 | 1.6E+05 |
| IL-21m_L77G | 2.42 | 1.3E+05 | 2.7E+04 | NA | 1.6E+05 |
| IL-21m_LA9G | 2.28 | 8.5E+04 | 3.5E+04 | 1.7E+04 | 1.6E+05 |
| IL-21m_E128R | 2.26 | 5.9E+04 | 3.8E+02 | 8.7E+01 | 1.6E+05 |
| IL-21m_L92R | 2.33 | 1.8E+04 | 6.0E+01 | 2.8E+01 | 1.6E+05 |
| IL-21m_F69Q | 2.53 | 9.7E+04 | 2.4E+04 | 1.3E+04 | 1.6E+05 |
| IL-21m_F69A | 2.63 | 4.2E+04 | 5.1E+02 | 8.0E+01 | 1.6E+05 |
| IL-21m_E131P | 2 | 1.4E+04 | 2.2E+02 | 5.1E+01 | 1.6E+05 |
| IL-21m_P116F | 2.04 | 3.1E+03 | 4.8E+01 | 3.0E+01 | 1.6E+05 |
| IL-21m_F129N | 2.19 | 9.7E+05 | 4.0E+03 | 1.0E+03 | 1.6E+05 |
| IL-21m_LA9T | 2.54 | 7.9E+04 | 3.4E+02 | 1.0E+02 | 1.6E+05 |
| IL-21m_N85I | 2.36 | 1.3E+05 | 3.7E+03 | 3.2E+02 | 1.6E+05 |
| IL-21m_L100A | 2.39 | 1.1E+05 | 4.6E+02 | 6.1E+01 | 1.6E+05 |
| IL-21m_L100G | 2.39 | 4.0E+04 | 3.3E+03 | 5.0E+02 | 1.6E+05 |
| IL-21m_M140N | 2.07 | 5.8E+04 | 3.7E+02 | 6.9E+01 | 1.6E+05 |
| IL-21m_Q73E | 2.43 | 3.9E+04 | 5.0E+02 | 1.1E+02 | 1.6E+05 |
| IL-21m_F129I | 2.16 | 9.6E+04 | 4.7E+02 | 1.3E+02 | 1.6E+05 |
| IL-21m_Q95E | 2.09 | 5.9E+02 | 2.6E+01 | 2.9E+01 | 1.6E+05 |
| IL-21m_L133D | 2.32 | 4.0E+05 | 2.4E+04 | 1.1E+04 | 1.6E+05 |
| IL-21m_I38D | 2.57 | 1.4E+05 | 4.1E+03 | 8.0E+02 | 1.6E+05 |
| IL-21m_Q143P | 2.51 | 9.3E+03 | 9.2E+01 | 3.1E+01 | 1.6E+05 |
| IL-21m_F69E | 2.66 | 6.5E+04 | 8.1E+03 | 1.9E+03 | 1.6E+05 |
| IL-21m_L100E | 2.32 | 7.6E+04 | 1.0E+03 | 2.6E+02 | 1.5E+05 |
| IL-21m_V39G | 2.34 | 1.3E+05 | 2.4E+03 | 2.4E+02 | 1.5E+05 |
| IL-21m_L35Q | 2.44 | 5.1E+04 | 4.2E+02 | 1.1E+02 | 1.5E+05 |
| IL-21m_P57K | 2.06 | 2.6E+05 | 6.9E+04 | 6.7E+03 | 1.5E+05 |
| IL-21m_I38Q | 2.43 | 1.1E+05 | 3.6E+03 | 3.1E+02 | 1.5E+05 |
| IL-21m_P57N | 2.02 | 8.4E+04 | 1.3E+04 | 7.7E+03 | 1.5E+05 |
| IL-21m_P101H | 2.12 | 1.7E+05 | 9.2E+02 | 1.2E+02 | 1.5E+05 |
| IL-21m_V93R | 2.09 | 2.1E+03 | 4.7E+01 | 1.6E+01 | 1.5E+05 |
| IL-21m_P126D | 2.25 | 2.0E+05 | 9.4E+04 | NA | 1.5E+05 |
| IL-21m_L77D | 2.37 | 9.7E+04 | NA | NA | 1.5E+05 |
| IL-21m_L137Q | 2.03 | 5.4E+04 | 7.4E+03 | 2.5E+03 | 1.5E+05 |
| IL-21m_R97P | 2.18 | 7.8E+04 | 2.4E+02 | 7.9E+01 | 1.5E+05 |
| IL-21m_A75R | 2.59 | 5.0E+05 | 1.3E+04 | 3.6E+02 | 1.5E+05 |
| IL-21m_P101V | 2.26 | 8.5E+04 | 4.4E+02 | 9.5E+01 | 1.5E+05 |
| IL-21m_K43P | 2.27 | 1.0E+05 | 1.2E+04 | 3.0E+03 | 1.5E+05 |
| IL-21m_L32E | 2.68 | 1.3E+05 | 2.1E+04 | 1.0E+04 | 1.5E+05 |
| IL-21m_L136R | 2.19 | 5.4E+04 | 2.3E+02 | 5.5E+01 | 1.5E+05 |
| IL-21m_R98W | 2.15 | 4.1E+03 | 4.1E+01 | 5.7E+01 | 1.5E+05 |
| IL-21m_M140R | 2.01 | 5.4E+04 | 2.2E+02 | 4.5E+01 | 1.5E+05 |
| IL-21m_L77S | 2.18 | 5.3E+04 | 7.4E+03 | 2.2E+03 | 1.5E+05 |
| IL-21m_V93K | 2.07 | 9.1E+04 | 2.6E+03 | 2.0E+02 | 1.5E+05 |
| IL-21m_D26G | 2.39 | 9.7E+03 | 8.2E+01 | 3.1E+01 | 1.5E+05 |
| IL-21m_LA9H | 2.19 | 2.7E+05 | 3.0E+04 | 1.8E+04 | 1.5E+05 |
| IL-21m_P126Q | 2.16 | 4.5E+04 | 4.4E+02 | 1.7E+02 | 1.5E+05 |
| IL-21m_Q73R | 2.21 | 3.8E+03 | 4.1E+01 | 1.3E+01 | 1.5E+05 |
| IL-21m_I89D | 2.33 | 6.9E+04 | 1.9E+03 | 1.6E+02 | 1.5E+05 |
| IL-21m_Y121P | 2.32 | 5.0E+04 | 1.1E+03 | 1.5E+02 | 1.5E+05 |
| IL-21m_F129H | 2.4 | 3.5E+04 | 2.4E+02 | 5.6E+01 | 1.5E+05 |
| IL-21m_R98Y | 2.43 | 1.4E+02 | 1.5E+01 | 4.0E+01 | 1.5E+05 |
| IL-21m_L77K | 2.12 | 1.2E+05 | NA | NA | 1.5E+05 |
| IL-21m_P57T | 2.1 | 8.5E+04 | 1.2E+04 | 8.7E+02 | 1.5E+05 |
| IL-21m_Q41P | 2.27 | 8.4E+04 | 6.0E+04 | 2.9E+04 | 1.5E+05 |
| IL-21m_R33P | 2.38 | 1.5E+05 | 5.1E+04 | 6.5E+03 | 1.5E+05 |
| IL-21m_Q73P | 2.32 | 7.1E+04 | 1.9E+04 | 8.8E+03 | 1.5E+05 |
| IL-21m_D26Q | 2.31 | 8.8E+03 | 4.4E+01 | 2.1E+01 | 1.5E+05 |
| IL-21m_R98G | 2.46 | 9.7E+04 | 7.8E+02 | 1.9E+02 | 1.5E+05 |
| IL-21m_LA9D | 2.31 | 1.4E+05 | 2.4E+04 | 1.3E+04 | 1.5E+05 |
| IL-21m_D26H | 2.62 | 2.2E+04 | 8.3E+01 | 2.9E+01 | 1.5E+05 |
| IL-21m_V60N | 2.04 | 4.2E+04 | 2.9E+02 | 7.2E+01 | 1.5E+05 |
| IL-21m_L130V | 2.35 | 1.1E+04 | 1.3E+02 | 4.8E+01 | 1.5E+05 |
| IL-21m_L137S | 2.1 | 5.0E+04 | 2.7E+02 | 5.7E+01 | 1.5E+05 |
| IL-21m_L32T | 2.46 | 1.0E+05 | NA | 2.6E+03 | 1.4E+05 |
| IL-21m_L96K | 2.16 | 2.3E+05 | NA | NA | 1.4E+05 |
| IL-21m_M140G | 2.24 | 3.5E+04 | 1.8E+03 | 1.9E+02 | 1.4E+05 |
| IL-21m_Y45P | 2.26 | 6.1E+04 | 1.5E+04 | 2.4E+03 | 1.4E+05 |
| IL-21m_Q73H | 2.12 | 3.3E+04 | 2.5E+02 | 5.2E+01 | 1.4E+05 |
| IL-21m_LA9E | 2.39 | 7.4E+04 | 2.8E+04 | 3.2E+04 | 1.4E+05 |
| IL-21m_L92P | 2.31 | 1.3E+05 | 2.6E+04 | NA | 1.4E+05 |
| IL-21m_L100F | 2.25 | 1.5E+04 | 6.6E+01 | 4.4E+01 | 1.4E+05 |
| IL-21m_M140T | 2.25 | 9.1E+03 | 9.8E+01 | 4.1E+01 | 1.4E+05 |
| IL-21m_L100K | 2.03 | 9.0E+04 | 2.1E+04 | 6.6E+02 | 1.4E+05 |
| IL-21m_M140P | 2.14 | 3.9E+04 | 3.9E+03 | 1.1E+03 | 1.4E+05 |
| IL-21m_R98A | 2 | 6.4E+03 | 4.9E+01 | 4.9E+01 | 1.4E+05 |
| IL-21m_D26P | 2.32 | 5.0E+03 | 3.8E+01 | 1.6E+01 | 1.4E+05 |
| IL-21m_F129K | 2.06 | 4.5E+04 | NA | NA | 1.4E+05 |
| IL-21m_I38H | 2.42 | 8.3E+04 | 4.9E+03 | 2.4E+02 | 1.4E+05 |
| IL-21m_V39E | 2.51 | 1.7E+05 | 2.7E+03 | 7.6E+02 | 1.4E+05 |
| IL-21m_L136D | 2.29 | 1.6E+05 | 1.8E+03 | 1.8E+02 | 1.4E+05 |
| IL-21m_N85W | 2.23 | 1.3E+05 | 1.3E+04 | NA | 1.4E+05 |
| IL-21m_L130A | 2.05 | 9.6E+04 | 1.1E+04 | 1.1E+03 | 1.4E+05 |
| IL-21m_P57E | 2.14 | 8.5E+04 | 1.3E+04 | 1.8E+03 | 1.4E+05 |
| IL-21m_L29P | 2.28 | 1.5E+04 | 1.1E+02 | 3.7E+01 | 1.4E+05 |
| IL-21m_L54D | 2.39 | 3.0E+04 | 2.3E+02 | 6.0E+01 | 1.4E+05 |
| IL-21m_I38R | 2.49 | 8.7E+04 | NA | NA | 1.4E+05 |
| IL-21m_L137P | 2.14 | 5.1E+04 | 8.5E+03 | 2.5E+03 | 1.4E+05 |
| IL-21m_R98P | 2.23 | 2.0E+05 | 1.9E+03 | 6.1E+02 | 1.4E+05 |
| IL-21m_Q41G | 2.06 | 1.3E+04 | 4.6E+01 | 2.6E+01 | 1.4E+05 |
| IL-21m_F72D | 2.62 | 1.2E+05 | NA | NA | 1.4E+05 |
| IL-21m_L35H | 2.5 | 2.3E+05 | 6.0E+02 | 1.1E+02 | 1.4E+05 |
| IL-21m_H34R | 2.03 | 3.3E+04 | 4.3E+02 | 8.0E+01 | 1.4E+05 |
| IL-21m_L96R | 2.19 | 6.1E+04 | NA | NA | 1.4E+05 |
| IL-21m_V39N | 2.48 | 8.3E+04 | 3.8E+03 | 5.0E+02 | 1.4E+05 |
| IL-21m_L92H | 2.36 | 4.7E+04 | 7.8E+01 | 2.1E+01 | 1.4E+05 |
| IL-21m_L77H | 2.12 | 8.3E+04 | NA | NA | 1.4E+05 |
| IL-21m_I38F | 2.16 | 1.2E+05 | 1.4E+03 | 1.8E+02 | 1.4E+05 |
| IL-21m_L32G | 2.67 | 1.4E+05 | 5.7E+04 | NA | 1.4E+05 |
| IL-21m_L42W | 2.1 | 1.3E+05 | 4.4E+04 | 4.6E+04 | 1.4E+05 |
| IL-21m_A56K | 2.61 | 1.8E+05 | 5.7E+03 | 1.5E+03 | 1.4E+05 |
| IL-21m_I38S | 2.27 | 7.0E+03 | 7.7E+01 | 4.3E+01 | 1.3E+05 |
| IL-21m_L133E | 2.13 | 7.7E+03 | 8.2E+01 | 8.3E+01 | 1.3E+05 |
| IL-21m_I38G | 2.67 | 1.1E+05 | 5.5E+03 | 3.9E+02 | 1.3E+05 |
| IL-21m_A56E | 2.66 | 6.0E+04 | 4.7E+03 | 1.4E+03 | 1.3E+05 |
| IL-21m_L49Q | 2.46 | 7.2E+04 | 6.0E+04 | NA | 1.3E+05 |
| IL-21m_P101L | 2.08 | 5.4E+04 | 4.3E+03 | 2.3E+02 | 1.3E+05 |
| IL-21m_F69S | 2.72 | 1.0E+05 | 1.2E+04 | NA | 1.3E+05 |
| IL-21m_I36P | 2.69 | 1.6E+05 | NA | NA | 1.3E+05 |
| IL-21m_I141R | 2.15 | 1.2E+04 | 1.3E+02 | 3.5E+01 | 1.3E+05 |
| IL-21m_E128S | 2.43 | 2.5E+04 | 2.4E+02 | 5.9E+01 | 1.3E+05 |
| IL-21m_L137R | 2.07 | 3.3E+04 | 2.0E+03 | 4.9E+02 | 1.3E+05 |
| IL-21m_I30R | 2.1 | 2.3E+05 | 3.4E+02 | 4.2E+01 | 1.3E+05 |
| IL-21m_D26Y | 2.38 | 1.3E+04 | 9.8E+01 | 2.8E+01 | 1.3E+05 |
| IL-21m_R31K | 2.29 | 1.1E+05 | 7.6E+04 | 1.0E+04 | 1.3E+05 |
| IL-21m_R98N | 2.42 | 5.0E+04 | 9.3E+01 | 6.6E+01 | 1.3E+05 |
| IL-21m_L32R | 2.67 | 7.3E+04 | 6.1E+03 | 1.9E+03 | 1.3E+05 |
| IL-21m_L29R | 2.28 | 3.7E+03 | 4.4E+01 | 1.9E+01 | 1.3E+05 |
| IL-21m_L92S | 2.53 | 1.1E+04 | 4.7E+01 | 3.1E+01 | 1.3E+05 |
| IL-21m_L92G | 2.46 | 2.1E+04 | 8.1E+01 | 5.0E+01 | 1.3E+05 |
| IL-21m_P101A | 2.02 | 8.2E+04 | 1.1E+03 | 2.0E+02 | 1.3E+05 |
| IL-21m_F69K | 2.27 | 4.9E+04 | 2.0E+04 | NA | 1.3E+05 |
| IL-21m_LA2G | 2.52 | 1.9E+05 | 3.3E+04 | NA | 1.3E+05 |
| IL-21m_H34S | 2.38 | 6.1E+02 | 3.2E+01 | 3.7E+01 | 1.3E+05 |
| IL-21m_L35S | 2.47 | 1.2E+05 | 5.1E+02 | 1.0E+02 | 1.3E+05 |
| IL-21m_L133P | 2.03 | 4.1E+04 | 6.7E+03 | 1.9E+03 | 1.3E+05 |
| IL-21m_Q95T | 2.2 | 1.4E+04 | 4.0E+01 | 4.0E+01 | 1.3E+05 |
| IL-21m_M140S | 2.19 | 8.9E+03 | 6.8E+01 | 3.3E+01 | 1.3E+05 |
| IL-21m_LA2N | 2.54 | 1.3E+05 | 1.4E+04 | 3.0E+03 | 1.3E+05 |
| IL-21m_N85V | 2.38 | 1.9E+04 | 2.9E+02 | 5.8E+01 | 1.3E+05 |
| IL-21m_LA9A | 2.28 | 1.2E+05 | 9.9E+03 | 1.5E+04 | 1.3E+05 |
| IL-21m_Q95W | 2.24 | 1.2E+05 | 9.0E+03 | 8.2E+02 | 1.3E+05 |
| IL-21m_I89E | 2.28 | 9.3E+04 | 8.8E+02 | 2.0E+02 | 1.3E+05 |
| IL-21m_M140Y | 2.42 | 6.9E+03 | 1.2E+02 | 5.3E+01 | 1.3E+05 |
| IL-21m_L32V | 2.39 | 2.2E+04 | 1.6E+02 | 6.2E+01 | 1.3E+05 |
| IL-21m_F72H | 2.54 | 2.6E+05 | 2.3E+04 | 4.1E+03 | 1.3E+05 |
| IL-21m_F129R | 2.04 | 1.3E+05 | 3.1E+04 | 3.8E+03 | 1.3E+05 |
| IL-21m_L136S | 2.42 | 5.8E+03 | 1.0E+02 | 3.2E+01 | 1.3E+05 |
| IL-21m_R27G | 2.2 | 7.4E+04 | 5.3E+01 | 3.2E+01 | 1.3E+05 |
| IL-21m_L35P | 2.68 | 1.5E+05 | 2.7E+04 | 1.1E+04 | 1.3E+05 |
| IL-21m_A56Q | 2.01 | 7.9E+04 | 4.2E+03 | 2.1E+02 | 1.3E+05 |
| IL-21m_L32A | 2.67 | 1.4E+05 | 1.4E+03 | 1.9E+02 | 1.3E+05 |
| IL-21m_S80P | 2.3 | 3.0E+03 | 4.3E+01 | 2.1E+01 | 1.3E+05 |
| IL-21m_Y121I | 2.43 | 9.8E+03 | 1.2E+02 | 3.5E+01 | 1.3E+05 |
| IL-21m_R98F | 2.26 | 2.9E+02 | 2.5E+01 | 4.4E+01 | 1.2E+05 |
| IL-21m_F129P | 2.11 | 1.8E+05 | 2.2E+04 | NA | 1.2E+05 |
| IL-21m_P79K | 2.21 | 9.6E+03 | 2.6E+02 | 8.3E+01 | 1.2E+05 |
| IL-21m_V39H | 2.27 | 1.6E+05 | 1.8E+04 | 6.0E+02 | 1.2E+05 |
| IL-21m_L96S | 2.23 | 1.1E+05 | 2.7E+04 | 3.3E+03 | 1.2E+05 |
| IL-21m_L77R | 2.19 | 1.2E+05 | 1.5E+04 | 1.7E+04 | 1.2E+05 |
| IL-21m_L42V | 2.28 | 9.8E+03 | 7.6E+01 | 3.5E+01 | 1.2E+05 |
| IL-21m_L32K | 2.68 | 9.7E+04 | 2.1E+04 | 7.0E+03 | 1.2E+05 |
| IL-21m_L77E | 2.62 | 4.8E+04 | 5.2E+03 | 2.5E+03 | 1.2E+05 |
| IL-21m_I141S | 2.22 | 8.4E+03 | 8.6E+01 | 2.4E+01 | 1.2E+05 |
| IL-21m_LA2P | 2.74 | 1.3E+05 | 1.1E+05 | NA | 1.2E+05 |
| IL-21m_I38K | 2.43 | 6.9E+04 | 3.9E+04 | 7.6E+02 | 1.2E+05 |
| IL-21m_L133H | 2.01 | 7.6E+04 | 4.0E+03 | 1.1E+02 | 1.2E+05 |
| IL-21m_I89P | 2.37 | 3.6E+03 | 2.9E+01 | 1.8E+01 | 1.2E+05 |
| IL-21m_P126W | 2.19 | 2.5E+05 | NA | NA | 1.2E+05 |
| IL-21m_V60K | 2.09 | 7.8E+04 | 2.5E+03 | 2.0E+02 | 1.2E+05 |
| IL-21m_E128W | 2.37 | 4.2E+04 | 5.0E+02 | 9.3E+01 | 1.2E+05 |
| IL-21m_F72Q | 2.61 | 5.6E+04 | 4.1E+03 | 6.1E+02 | 1.2E+05 |
| IL-21m_L32N | 2.7 | 1.5E+05 | NA | 1.3E+04 | 1.2E+05 |
| IL-21m_R98I | 2.18 | 4.4E+03 | 4.2E+01 | 3.5E+01 | 1.2E+05 |
| IL-21m_A68K | 2.19 | 1.4E+05 | 1.9E+04 | 3.7E+03 | 1.2E+05 |
| IL-21m_LA9N | 2.31 | 7.2E+04 | 2.5E+04 | NA | 1.2E+05 |
| IL-21m_I44P | 2.61 | 1.9E+03 | 2.3E+01 | 1.6E+01 | 1.2E+05 |
| IL-21m_Q95D | 2.16 | 3.5E+03 | 3.4E+01 | 2.5E+01 | 1.2E+05 |
| IL-21m_L35E | 2.55 | 6.1E+04 | 4.7E+03 | 1.6E+03 | 1.2E+05 |
| IL-21m_I89T | 2.31 | 1.2E+04 | 1.0E+02 | 3.3E+01 | 1.2E+05 |
| IL-21m_L96P | 2.27 | 1.0E+05 | 4.2E+04 | NA | 1.2E+05 |
| IL-21m_V39R | 2.45 | 9.0E+04 | 1.1E+04 | 2.9E+03 | 1.2E+05 |
| IL-21m_LA2E | 2.39 | 6.9E+04 | 7.5E+03 | 1.8E+03 | 1.2E+05 |
| IL-21m_V39P | 2.45 | 8.8E+04 | 3.9E+04 | NA | 1.2E+05 |
| IL-21m_L92T | 2.22 | 1.2E+04 | 8.7E+01 | 3.8E+01 | 1.2E+05 |
| IL-21m_H34D | 2.11 | 8.5E+03 | 3.0E+02 | 1.9E+02 | 1.2E+05 |
| IL-21m_L130W | 2.33 | 4.3E+04 | 2.4E+03 | 5.6E+02 | 1.2E+05 |
| IL-21m_L100D | 2.1 | 2.1E+05 | 3.0E+04 | 1.3E+03 | 1.2E+05 |
| IL-21m_L137A | 2.11 | 4.8E+03 | 4.1E+01 | 3.0E+01 | 1.2E+05 |
| IL-21m_S120P | 2.08 | 4.6E+04 | 2.6E+02 | 5.0E+01 | 1.2E+05 |
| IL-21m_L32Q | 2.64 | 1.4E+05 | 2.4E+04 | 2.9E+03 | 1.2E+05 |
| IL-21m_F129E | 2.11 | 7.4E+04 | 2.3E+04 | 2.0E+04 | 1.2E+05 |
| IL-21m_L35N | 2.61 | 5.5E+03 | 3.4E+01 | 3.0E+01 | 1.2E+05 |
| IL-21m_I89G | 2.36 | 4.2E+04 | 4.8E+03 | 2.6E+02 | 1.1E+05 |
| IL-21m_I89F | 2.32 | 7.8E+03 | 1.4E+02 | 4.3E+01 | 1.1E+05 |
| IL-21m_L130D | 2.03 | 3.4E+04 | 6.8E+03 | 1.6E+03 | 1.1E+05 |
| IL-21m_M140Q | 2.17 | 3.3E+03 | 4.5E+01 | 2.3E+01 | 1.1E+05 |
| IL-21m_R98D | 2.32 | 1.3E+05 | 2.1E+04 | NA | 1.1E+05 |
| IL-21m_R132W | 2.29 | 3.9E+04 | 2.3E+02 | 5.1E+01 | 1.1E+05 |
| IL-21m_L77P | 2.57 | 1.4E+04 | 1.7E+02 | 5.7E+01 | 1.1E+05 |
| IL-21m_I89R | 2.01 | 1.0E+05 | 5.0E+03 | 6.6E+02 | 1.1E+05 |
| IL-21m_H142P | 2.4 | 4.1E+04 | 9.4E+02 | 1.4E+02 | 1.1E+05 |
| IL-21m_L32P | 2.73 | 7.8E+04 | 9.4E+03 | 2.0E+03 | 1.1E+05 |
| IL-21m_L96H | 2.06 | 4.8E+04 | 2.0E+04 | NA | 1.1E+05 |
| IL-21m_L32W | 2.22 | 3.3E+03 | 3.3E+01 | 1.6E+01 | 1.1E+05 |
| IL-21m_V39Q | 2.12 | 1.8E+04 | 4.7E+02 | 1.1E+02 | 1.1E+05 |
| IL-21m_L35R | 2.33 | 1.2E+05 | NA | NA | 1.1E+05 |
| IL-21m_L54P | 2.26 | 5.3E+04 | 8.8E+03 | 7.3E+02 | 1.1E+05 |
| IL-21m_D26K | 2.26 | 7.4E+03 | 5.5E+01 | 1.8E+01 | 1.1E+05 |
| IL-21m_L35F | 2.09 | 2.3E+03 | 2.4E+01 | 1.6E+01 | 1.1E+05 |
| IL-21m_F72E | 2.65 | 7.1E+04 | 1.5E+04 | NA | 1.1E+05 |
| IL-21m_L136K | 2.22 | 3.2E+04 | 1.4E+03 | 1.3E+02 | 1.1E+05 |
| IL-21m_F72S | 2.56 | 2.8E+05 | 2.0E+03 | 2.0E+02 | 1.1E+05 |
| IL-21m_R27N | 2.02 | 2.1E+05 | 9.8E+01 | 4.5E+01 | 1.1E+05 |
| IL-21m_R31L | 2.43 | 9.8E+04 | 1.6E+04 | 3.3E+03 | 1.1E+05 |
| IL-21m_V39K | 2.44 | 1.1E+05 | 2.1E+04 | 1.3E+04 | 1.1E+05 |
| IL-21m_L92A | 2.28 | 2.1E+03 | 1.9E+01 | 1.7E+01 | 1.1E+05 |
| IL-21m_K78P | 2.23 | 5.0E+04 | 6.3E+02 | 8.8E+01 | 1.1E+05 |
| IL-21m_LA2A | 2.38 | 1.6E+05 | 6.6E+02 | 9.9E+01 | 1.1E+05 |
| IL-21m_D119P | 2.05 | 3.5E+03 | 6.5E+01 | 2.5E+01 | 1.1E+05 |
| IL-21m_R98T | 2.39 | 4.6E+04 | 5.5E+01 | 3.1E+01 | 1.1E+05 |
| IL-21m_A75D | 2.41 | 2.6E+04 | 1.8E+02 | 5.7E+01 | 1.1E+05 |
| IL-21m_A56D | 2.05 | 5.2E+04 | 7.6E+03 | 1.3E+03 | 1.1E+05 |
| IL-21m_R27S | 2.12 | 3.1E+04 | 3.0E+01 | 2.5E+01 | 1.1E+05 |
| IL-21m_L92D | 2.37 | 1.6E+05 | 1.4E+04 | 3.0E+03 | 1.1E+05 |
| IL-21m_A68E | 2.49 | 2.5E+04 | 4.3E+02 | 1.1E+02 | 1.1E+05 |
| IL-21m_R27K | 2.55 | 2.4E+04 | 6.9E+01 | 3.7E+01 | 1.0E+05 |
| IL-21m_M140V | 2.12 | 1.9E+04 | 1.3E+02 | 3.5E+01 | 1.0E+05 |
| IL-21m_Y45D | 2.31 | 1.7E+04 | 2.5E+02 | 6.3E+01 | 1.0E+05 |
| IL-21m_V93N | 2.38 | 6.6E+03 | 5.1E+01 | 2.7E+01 | 1.0E+05 |
| IL-21m_P57A | 2.01 | 7.8E+03 | 8.0E+01 | 3.7E+01 | 1.0E+05 |
| IL-21m_Y121K | 2.43 | 5.9E+04 | 6.8E+02 | 1.0E+02 | 1.0E+05 |
| IL-21m_Y121D | 2.49 | 8.6E+03 | 8.5E+01 | 6.2E+01 | 1.0E+05 |
| IL-21m_I89H | 2.14 | 6.0E+04 | 1.8E+03 | 8.9E+01 | 1.0E+05 |
| IL-21m_P25K | 2 | 6.6E+02 | 1.6E+01 | 7.0E+00 | 1.0E+05 |
| IL-21m_I38N | 2.58 | 1.6E+04 | 6.7E+01 | 3.5E+01 | 1.0E+05 |
| IL-21m_Q41D | 2.38 | 5.0E+03 | 4.1E+01 | 3.3E+01 | 1.0E+05 |
| IL-21m_I89K | 2.3 | 1.8E+05 | 1.0E+04 | 1.9E+03 | 1.0E+05 |
| IL-21m_P126S | 2.04 | 6.9E+03 | 8.1E+01 | 4.8E+01 | 1.0E+05 |
| IL-21m_S117F | 2.15 | 4.2E+03 | 5.7E+01 | 4.6E+01 | 1.0E+05 |
| IL-21m_A75K | 2.2 | 4.5E+04 | 5.5E+02 | 9.2E+01 | 9.9E+04 |
| IL-21m_H34G | 2.28 | 2.9E+03 | 5.5E+01 | 5.4E+01 | 9.9E+04 |
| IL-21m_A68T | 2.25 | 2.9E+03 | 4.2E+01 | 2.4E+01 | 9.9E+04 |
| IL-21m_V60D | 2.31 | 1.4E+04 | 9.4E+01 | 4.5E+01 | 9.9E+04 |
| IL-21m_I89S | 2.42 | 4.0E+04 | 1.2E+03 | 9.1E+01 | 9.9E+04 |
| IL-21m_A68H | 2.19 | 4.4E+04 | 1.9E+03 | 1.6E+02 | 9.8E+04 |
| IL-21m_R97W | 2.13 | 4.8E+04 | 3.6E+02 | 1.1E+02 | 9.8E+04 |
| IL-21m_Q73L | 2.07 | 2.5E+03 | 4.1E+01 | 2.6E+01 | 9.8E+04 |
| IL-21m_L28D | 2.14 | 3.9E+03 | 3.6E+01 | 4.1E+01 | 9.8E+04 |
| IL-21m_Y45G | 2.43 | 1.4E+04 | 8.4E+01 | 4.0E+01 | 9.8E+04 |
| IL-21m_I141G | 2.23 | 3.5E+04 | 3.4E+02 | 6.7E+01 | 9.7E+04 |
| IL-21m_M140F | 2.29 | 6.2E+03 | 6.7E+01 | 2.9E+01 | 9.6E+04 |
| IL-21m_L29K | 2.52 | 2.0E+03 | 3.5E+01 | 1.8E+01 | 9.6E+04 |
| IL-21m_D26E | 2.04 | 3.2E+03 | 2.7E+01 | 1.6E+01 | 9.5E+04 |
| IL-21m_R27T | 2.14 | 2.1E+04 | 2.9E+01 | 2.2E+01 | 9.5E+04 |
| IL-21m_L28S | 2.23 | 7.2E+02 | 2.2E+01 | 1.4E+01 | 9.5E+04 |
| IL-21m_P126F | 2.22 | 2.0E+04 | 2.5E+02 | 8.0E+01 | 9.4E+04 |
| IL-21m_L92E | 2.25 | 4.9E+04 | 2.2E+03 | 5.7E+02 | 9.4E+04 |
| IL-21m_I141N | 2.17 | 2.4E+04 | 3.3E+02 | 4.8E+01 | 9.4E+04 |
| IL-21m_V60R | 2.06 | 3.3E+04 | 1.7E+03 | 1.3E+02 | 9.2E+04 |
| IL-21m_V60H | 2.11 | 3.9E+04 | 3.3E+02 | 6.3E+01 | 9.2E+04 |
| IL-21m_Q73V | 2.19 | 1.0E+03 | 2.5E+01 | 1.8E+01 | 9.1E+04 |
| IL-21m_A68G | 2.56 | 1.5E+04 | 6.2E+01 | 2.6E+01 | 9.1E+04 |
| IL-21m_I141P | 2.41 | 3.3E+04 | 2.7E+03 | 8.1E+02 | 9.1E+04 |
| IL-21m_I141Q | 2.02 | 2.4E+03 | 3.4E+01 | 1.8E+01 | 9.1E+04 |
| IL-21m_I89N | 2.3 | 6.1E+04 | 2.4E+03 | 1.7E+02 | 9.0E+04 |
| IL-21m_L28N | 2.26 | 9.9E+02 | 2.7E+01 | 1.7E+01 | 8.9E+04 |
| IL-21m_L96W | 2.16 | 5.9E+04 | 9.8E+02 | 1.8E+02 | 8.9E+04 |
| IL-21m_Q95H | 2.21 | 5.1E+03 | 4.5E+01 | 2.2E+01 | 8.8E+04 |
| IL-21m_Y45T | 2.32 | 3.9E+03 | 4.8E+01 | 3.1E+01 | 8.8E+04 |
| IL-21m_E40K | 2.09 | 8.0E+02 | 2.0E+01 | 7.0E+00 | 8.8E+04 |
| IL-21m_L32S | 2.78 | 7.2E+04 | 1.9E+04 | NA | 8.8E+04 |
| IL-21m_LA2D | 2.29 | 7.7E+04 | 1.8E+04 | NA | 8.8E+04 |
| IL-21m_A75F | 2.03 | 3.9E+03 | 5.9E+01 | 2.7E+01 | 8.7E+04 |
| IL-21m_R27D | 2 | 1.2E+05 | 1.5E+03 | 1.4E+02 | 8.7E+04 |
| IL-21m_L49V | 2.19 | 2.2E+03 | 3.8E+01 | 1.7E+01 | 8.6E+04 |
| IL-21m_M140K | 2.34 | 2.1E+04 | 1.2E+02 | 2.3E+01 | 8.6E+04 |
| IL-21m_F129M | 2.26 | 7.2E+02 | 3.3E+01 | 1.5E+01 | 8.5E+04 |
| IL-21m_L130E | 2.33 | 7.8E+04 | 7.7E+03 | 2.3E+03 | 8.5E+04 |
| IL-21m_L96Y | 2.09 | 9.1E+02 | 3.0E+01 | 1.6E+01 | 8.4E+04 |
| IL-21m_L136A | 2.22 | 4.1E+03 | 6.5E+01 | 2.7E+01 | 8.4E+04 |
| IL-21m_R132P | 2.16 | 6.1E+04 | 7.0E+03 | 8.5E+02 | 8.4E+04 |
| IL-21m_P126G | 2.19 | 1.1E+04 | 1.7E+02 | 6.1E+01 | 8.4E+04 |
| IL-21m_D119K | 2.15 | 6.1E+02 | 3.1E+01 | 1.1E+01 | 8.3E+04 |
| IL-21m_L28G | 2.34 | 1.4E+03 | 2.6E+01 | 1.9E+01 | 8.2E+04 |
| IL-21m_L96E | 2.37 | 1.0E+05 | 4.5E+04 | NA | 8.1E+04 |
| IL-21m_L77A | 2.05 | 3.5E+04 | 3.5E+03 | 4.5E+02 | 8.1E+04 |
| IL-21m_F72K | 2.36 | 1.2E+05 | 2.3E+04 | NA | 8.1E+04 |
| IL-21m_V60E | 2.3 | 5.2E+03 | 5.4E+01 | 2.3E+01 | 8.1E+04 |
| IL-21m_L130T | 2.08 | 3.5E+04 | 3.0E+03 | 3.1E+02 | 8.0E+04 |
| IL-21m_M140A | 2.1 | 1.9E+04 | 1.4E+02 | 4.5E+01 | 7.9E+04 |
| IL-21m_L77F | 2.02 | 3.3E+03 | 4.4E+01 | 2.0E+01 | 7.8E+04 |
| IL-21m_Y121Q | 2.14 | 3.5E+03 | 2.0E+02 | 6.7E+01 | 7.8E+04 |
| IL-21m_Q73I | 2.17 | 1.4E+03 | 3.5E+01 | 1.9E+01 | 7.8E+04 |
| IL-21m_I89Q | 2.01 | 8.2E+04 | 1.7E+03 | 1.6E+02 | 7.7E+04 |
| IL-21m_Q73S | 2.29 | 2.7E+03 | 2.5E+01 | 1.3E+01 | 7.7E+04 |
| IL-21m_E128K | 2.64 | 8.8E+02 | 3.3E+01 | 1.4E+01 | 7.6E+04 |
| IL-21m_Y121N | 2.45 | 3.5E+03 | 4.7E+01 | 2.8E+01 | 7.5E+04 |
| IL-21m_I141K | 2.34 | 1.9E+03 | 4.0E+01 | 1.0E+01 | 7.5E+04 |
| IL-21m_L136Y | 2.27 | 1.4E+03 | 3.3E+01 | 1.3E+01 | 7.4E+04 |
| IL-21m_F72T | 2.13 | 3.1E+04 | 1.4E+03 | 2.3E+02 | 7.4E+04 |
| IL-21m_V93E | 2.23 | 4.0E+03 | 1.1E+02 | 4.2E+01 | 7.4E+04 |
| IL-21m_P116H | 2.12 | 1.9E+03 | 3.9E+01 | 1.6E+01 | 7.4E+04 |
| IL-21m_Q41S | 2.17 | 9.5E+02 | 1.5E+01 | 1.2E+01 | 7.4E+04 |
| IL-21m_P25N | 2.58 | 7.6E+02 | 1.7E+01 | 1.1E+01 | 7.3E+04 |
| IL-21m_L145P | 2.17 | 1.2E+03 | 2.4E+01 | 1.3E+01 | 7.3E+04 |
| IL-21m_L35A | 2.45 | 6.7E+03 | 3.6E+01 | 2.3E+01 | 7.3E+04 |
| IL-21m_I30K | 2.36 | 1.7E+05 | 2.9E+02 | 3.5E+01 | 7.2E+04 |
| IL-21m_R98M | 2.02 | 5.8E+01 | 1.0E+01 | 1.8E+01 | 7.2E+04 |
| IL-21m_N85M | 2.2 | 4.8E+03 | 9.0E+01 | 3.7E+01 | 7.1E+04 |
| IL-21m_E65K | 2.02 | 3.9E+02 | 2.8E+01 | 9.0E+00 | 7.1E+04 |
| IL-21m_I89A | 2.55 | 3.6E+03 | 4.7E+01 | 1.6E+01 | 7.1E+04 |
| IL-21m_H34A | 2.02 | 2.4E+02 | 2.7E+01 | 3.2E+01 | 7.1E+04 |
| IL-21m_L133I | 2.2 | 2.0E+03 | 3.0E+01 | 2.2E+01 | 7.0E+04 |
| IL-21m_L29H | 2.37 | 8.9E+02 | 1.9E+01 | 9.0E+00 | 7.0E+04 |
| IL-21m_D91K | 2.07 | 3.5E+02 | 1.9E+01 | 6.0E+00 | 6.9E+04 |
| IL-21m_T125W | 2.35 | 4.6E+02 | 2.4E+01 | 1.5E+01 | 6.8E+04 |
| IL-21m_M140I | 2.19 | 1.1E+03 | 2.0E+01 | 2.3E+01 | 6.7E+04 |
| IL-21m_L137M | 2.25 | 4.5E+02 | 2.5E+01 | 1.3E+01 | 6.7E+04 |
| IL-21m_Q73A | 2.14 | 1.9E+03 | 2.3E+01 | 1.3E+01 | 6.6E+04 |
| IL-21m_L32I | 2.17 | 6.3E+02 | 2.5E+01 | 1.4E+01 | 6.6E+04 |
| IL-21m_D26S | 2.23 | 1.4E+03 | 2.1E+01 | 1.0E+01 | 6.5E+04 |
| IL-21m_L54K | 2.34 | 3.5E+02 | 2.3E+01 | 1.3E+01 | 6.5E+04 |
| IL-21m_V60A | 2.23 | 5.7E+02 | 2.6E+01 | 1.2E+01 | 6.5E+04 |
| IL-21m_L96Q | 2.18 | 9.8E+04 | 7.3E+04 | 7.0E+03 | 6.4E+04 |
| IL-21m_A68Q | 2.19 | 3.1E+03 | 5.1E+01 | 3.9E+01 | 6.4E+04 |
| IL-21m_R98L | 2.18 | 8.2E+01 | 1.4E+01 | 2.2E+01 | 6.4E+04 |
| IL-21m_D119H | 2.2 | 7.3E+02 | 2.1E+01 | 7.0E+00 | 6.4E+04 |
| IL-21m_Q73T | 2.35 | 3.5E+02 | 1.8E+01 | 1.1E+01 | 6.3E+04 |
| IL-21m_H34N | 2.12 | 1.9E+02 | 2.0E+01 | 2.1E+01 | 6.3E+04 |
| IL-21m_E131W | 2 | 3.2E+02 | 2.2E+01 | 1.0E+01 | 6.3E+04 |
| IL-21m_Y45E | 2.4 | 6.9E+02 | 2.6E+01 | 1.5E+01 | 6.2E+04 |
| IL-21m_K123I | 2.18 | 4.1E+03 | 4.6E+01 | 3.1E+01 | 6.2E+04 |
| IL-21m_I141F | 2.25 | 8.0E+02 | 2.1E+01 | 1.2E+01 | 6.2E+04 |
| IL-21m_D119V | 2.13 | 3.9E+02 | 2.3E+01 | 1.2E+01 | 6.2E+04 |
| IL-21m_L133V | 2.39 | 1.1E+03 | 2.9E+01 | 2.0E+01 | 6.2E+04 |
| IL-21m_E128H | 2.32 | 2.5E+03 | 6.4E+01 | 1.7E+01 | 6.1E+04 |
| IL-21m_E128T | 2.37 | 1.9E+03 | 3.9E+01 | 2.7E+01 | 6.1E+04 |
| IL-21m_F72I | 2.22 | 2.6E+03 | 3.8E+01 | 1.6E+01 | 6.1E+04 |
| IL-21m_E122F | 2.12 | 9.0E+02 | 3.3E+01 | 2.6E+01 | 6.1E+04 |
| IL-21m_A68S | 2.28 | 3.1E+03 | 5.2E+01 | 2.9E+01 | 6.1E+04 |
| IL-21m_L28P | 2.53 | 7.9E+02 | 3.2E+01 | 2.5E+01 | 6.0E+04 |
| IL-21m_F129L | 2.3 | 3.0E+03 | 4.1E+01 | 2.7E+01 | 6.0E+04 |
| IL-21m_D119M | 2.08 | 2.6E+02 | 2.0E+01 | 9.0E+00 | 5.8E+04 |
| IL-21m_I141W | 2.44 | 4.9E+02 | 1.9E+01 | 1.2E+01 | 5.8E+04 |
| IL-21m_Q73N | 2.41 | 1.1E+04 | 7.8E+01 | 2.8E+01 | 5.8E+04 |
| IL-21m_R97L | 2.18 | 8.0E+03 | 9.5E+01 | 3.8E+01 | 5.8E+04 |
| IL-21m_R97F | 2.37 | 8.0E+03 | 5.5E+01 | 2.8E+01 | 5.7E+04 |
| IL-21m_Q73K | 2.3 | 1.5E+04 | 2.6E+02 | 3.9E+01 | 5.7E+04 |
| IL-21m_L136Q | 2.32 | 4.1E+02 | 2.2E+01 | 9.0E+00 | 5.7E+04 |
| IL-21m_A75N | 2.53 | 3.0E+02 | 1.9E+01 | 1.3E+01 | 5.6E+04 |
| IL-21m_L136M | 2.08 | 2.7E+02 | 2.0E+01 | 1.0E+01 | 5.6E+04 |
| IL-21m_Y121R | 2.43 | 3.9E+04 | 7.0E+02 | 8.5E+01 | 5.6E+04 |
| IL-21m_P126K | 2.07 | 3.3E+02 | 1.8E+01 | 1.0E+01 | 5.5E+04 |
| IL-21m_I141H | 2.49 | 4.6E+02 | 1.6E+01 | 8.0E+00 | 5.4E+04 |
| IL-21m_L100M | 2.22 | 1.3E+03 | 2.4E+01 | 1.8E+01 | 5.4E+04 |
| IL-21m_L28K | 2.57 | 5.7E+02 | 2.0E+01 | 8.0E+00 | 5.3E+04 |
| IL-21m_P25Y | 2.71 | 4.0E+02 | 1.8E+01 | 1.4E+01 | 5.3E+04 |
| IL-21m_E128F | 2.29 | 2.5E+03 | 4.2E+01 | 1.9E+01 | 5.3E+04 |
| IL-21m_F69W | 2.19 | 3.7E+02 | 2.0E+01 | 1.1E+01 | 5.3E+04 |
| IL-21m_I141T | 2.35 | 3.5E+02 | 2.2E+01 | 1.2E+01 | 5.2E+04 |
| IL-21m_D119Q | 2.05 | 2.1E+02 | 1.6E+01 | 7.0E+00 | 5.1E+04 |
| IL-21m_Y121H | 2.58 | 3.1E+02 | 1.7E+01 | 8.0E+00 | 5.1E+04 |
| IL-21m_L35V | 2.36 | 3.2E+02 | 2.0E+01 | 1.1E+01 | 5.1E+04 |
| IL-21m_L29N | 2.43 | 5.7E+02 | 2.1E+01 | 1.5E+01 | 5.1E+04 |
| IL-21m_L28A | 2.28 | 4.1E+02 | 1.9E+01 | 1.1E+01 | 5.0E+04 |
| IL-21m_L136W | 2.51 | 1.3E+03 | 2.5E+01 | 1.2E+01 | 5.0E+04 |
| IL-21m_F129V | 2.23 | 1.0E+03 | 4.0E+01 | 1.8E+01 | 5.0E+04 |
| IL-21m_Y121T | 2.26 | 4.8E+03 | 5.1E+01 | 3.4E+01 | 5.0E+04 |
| IL-21m_LA9Y | 2.17 | 3.6E+02 | 2.1E+01 | 1.1E+01 | 4.9E+04 |
| IL-21m_Y121V | 2.05 | 2.4E+03 | 3.7E+01 | 2.0E+01 | 4.9E+04 |
| IL-21m_A68M | 2.31 | 3.4E+02 | 2.3E+01 | 1.5E+01 | 4.8E+04 |
| IL-21m_Y121M | 2.33 | 7.7E+02 | 3.1E+01 | 1.3E+01 | 4.8E+04 |
| IL-21m_I30V | 2.13 | 4.1E+02 | 1.4E+01 | 9.0E+00 | 4.7E+04 |
| IL-21m_I36K | 2.26 | 2.6E+02 | 1.8E+01 | 1.0E+01 | 4.7E+04 |
| IL-21m_L133G | 2.16 | 1.9E+02 | 1.4E+01 | 8.0E+00 | 4.7E+04 |
| IL-21m_A68V | 2.11 | 5.9E+02 | 3.2E+01 | 1.6E+01 | 4.6E+04 |
| IL-21m_A102H | 2.17 | 1.6E+02 | 1.3E+01 | 5.0E+00 | 4.6E+04 |
| IL-21m_E128Y | 2.29 | 5.1E+02 | 2.3E+01 | 1.1E+01 | 4.6E+04 |
| IL-21m_M140W | 2.36 | 1.4E+02 | 1.3E+01 | 7.0E+00 | 4.6E+04 |
| IL-21m_L35M | 2.09 | 4.3E+02 | 1.4E+01 | 1.0E+01 | 4.5E+04 |
| IL-21m_L96V | 2.36 | 4.9E+02 | 1.8E+01 | 9.0E+00 | 4.5E+04 |
| IL-21m_L130F | 2.11 | 4.3E+02 | 2.0E+01 | 1.3E+01 | 4.5E+04 |
| IL-21m_R132F | 2.2 | 2.4E+03 | 4.1E+01 | 3.4E+01 | 4.4E+04 |
| IL-21m_L29S | 2.58 | 2.7E+02 | 1.4E+01 | 8.0E+00 | 4.3E+04 |
| IL-21m_V93F | 2.16 | 3.8E+02 | 2.6E+01 | 1.4E+01 | 4.3E+04 |
| IL-21m_V93G | 2.33 | 1.0E+03 | 2.2E+01 | 1.9E+01 | 4.2E+04 |
| IL-21m_F72V | 2.47 | 1.7E+03 | 3.9E+01 | 1.8E+01 | 4.2E+04 |
| IL-21m_E128G | 2.32 | 1.5E+03 | 3.8E+01 | 1.6E+01 | 4.2E+04 |
| IL-21m_L130M | 2.19 | 1.7E+03 | 4.0E+01 | 1.6E+01 | 4.2E+04 |
| IL-21m_R99W | 2.41 | 4.4E+03 | 4.3E+01 | 3.5E+01 | 4.1E+04 |
| IL-21m_E40H | 2.01 | 2.3E+02 | 2.0E+01 | 9.0E+00 | 4.1E+04 |
| IL-21m_L54T | 2.06 | 1.2E+03 | 3.4E+01 | 1.7E+01 | 4.1E+04 |
| IL-21m_V39S | 2.41 | 3.3E+02 | 2.2E+01 | 1.2E+01 | 4.1E+04 |
| IL-21m_L92V | 2.16 | 2.4E+03 | 3.8E+01 | 1.3E+01 | 4.0E+04 |
| IL-21m_I90R | 2.06 | 6.9E+01 | 1.1E+01 | 4.0E+00 | 4.0E+04 |
| IL-21m_R124W | 2.26 | 2.0E+03 | 3.2E+01 | 4.2E+01 | 4.0E+04 |
| IL-21m_A75S | 2.14 | 4.3E+02 | 2.6E+01 | 1.6E+01 | 3.9E+04 |
| IL-21m_E46K | 2.32 | 1.4E+02 | 1.3E+01 | 6.0E+00 | 3.9E+04 |
| IL-21m_R132V | 2.19 | 8.0E+02 | 2.5E+01 | 1.4E+01 | 3.9E+04 |
| IL-21m_A67G | 2.05 | 5.1E+02 | 2.8E+01 | 1.4E+01 | 3.9E+04 |
| IL-21m_D48V | 2.48 | 2.2E+02 | 1.6E+01 | 1.0E+01 | 3.9E+04 |
| IL-21m_Q95K | 2.4 | 5.1E+02 | 2.4E+01 | 1.0E+01 | 3.8E+04 |
| IL-21m_D119T | 2.17 | 1.5E+02 | 1.5E+01 | 7.0E+00 | 3.8E+04 |
| IL-21m_Q58P | 2.1 | 3.4E+02 | 1.9E+01 | 1.1E+01 | 3.8E+04 |
| IL-21m_L29D | 2.13 | 3.8E+02 | 1.7E+01 | 1.7E+01 | 3.8E+04 |
| IL-21m_I36H | 2.36 | 2.5E+02 | 1.5E+01 | 7.0E+00 | 3.8E+04 |
| IL-21m_D26N | 2.31 | 2.6E+02 | 1.3E+01 | 6.0E+00 | 3.8E+04 |
| IL-21m_H144P | 2.28 | 1.4E+03 | 3.3E+01 | 1.6E+01 | 3.7E+04 |
| IL-21m_R132Y | 2.14 | 8.6E+02 | 3.2E+01 | 1.7E+01 | 3.6E+04 |
| IL-21m_L92I | 2.22 | 1.0E+03 | 2.9E+01 | 2.0E+01 | 3.6E+04 |
| IL-21m_E128A | 2.06 | 3.6E+02 | 2.2E+01 | 1.2E+01 | 3.6E+04 |
| IL-21m_H144F | 2.32 | 8.2E+02 | 2.5E+01 | 1.4E+01 | 3.6E+04 |
| IL-21m_K123D | 2.1 | 8.4E+02 | 3.3E+01 | 2.1E+01 | 3.5E+04 |
| IL-21m_D48H | 2.05 | 1.3E+02 | 1.2E+01 | 5.0E+00 | 3.5E+04 |
| IL-21m_D119S | 2.06 | 1.8E+02 | 1.6E+01 | 7.0E+00 | 3.5E+04 |
| IL-21m_V93Y | 2.2 | 3.4E+03 | 3.4E+01 | 2.6E+01 | 3.4E+04 |
| IL-21m_I141A | 2.22 | 3.4E+02 | 1.6E+01 | 9.0E+00 | 3.4E+04 |
| IL-21m_I36G | 2.55 | 3.0E+02 | 1.9E+01 | 1.0E+01 | 3.4E+04 |
| IL-21m_L136N | 2.18 | 5.5E+02 | 2.3E+01 | 1.0E+01 | 3.3E+04 |
| IL-21m_P126M | 2.38 | 3.9E+02 | 1.9E+01 | 1.3E+01 | 3.3E+04 |
| IL-21m_Y45K | 2.37 | 3.5E+02 | 1.8E+01 | 1.0E+01 | 3.3E+04 |
| IL-21m_L29Q | 2.39 | 3.2E+02 | 1.7E+01 | 1.0E+01 | 3.2E+04 |
| IL-21m_L136T | 2.16 | 3.4E+02 | 2.5E+01 | 1.3E+01 | 3.2E+04 |
| IL-21m_D50Q | 2.56 | 8.7E+01 | 1.0E+01 | 5.0E+00 | 3.2E+04 |
| IL-21m_H144G | 2.16 | 6.9E+02 | 2.3E+01 | 1.4E+01 | 3.2E+04 |
| IL-21m_P116V | 2.04 | 3.4E+02 | 2.0E+01 | 1.4E+01 | 3.2E+04 |
| IL-21m_L28W | 2.69 | 1.4E+02 | 1.2E+01 | 7.0E+00 | 3.2E+04 |
| IL-21m_D119A | 2.1 | 1.3E+02 | 1.4E+01 | 5.0E+00 | 3.2E+04 |
| IL-21m_R99Y | 2.12 | 2.0E+03 | 2.7E+01 | 1.6E+01 | 3.2E+04 |
| IL-21m_F88G | 2.51 | 4.5E+02 | 2.1E+01 | 1.1E+01 | 3.1E+04 |
| IL-21m_E131K | 2.22 | 1.4E+02 | 1.6E+01 | 8.0E+00 | 3.1E+04 |
| IL-21m_P126L | 2.34 | 6.2E+02 | 2.6E+01 | 1.5E+01 | 3.1E+04 |
| IL-21m_Y45S | 2.4 | 3.0E+02 | 1.7E+01 | 8.0E+00 | 3.1E+04 |
| IL-21m_L130I | 2.21 | 2.7E+02 | 2.0E+01 | 1.2E+01 | 3.1E+04 |
| IL-21m_L28H | 2.38 | 2.6E+02 | 1.2E+01 | 6.0E+00 | 3.1E+04 |
| IL-21m_F88S | 2.14 | 2.3E+02 | 1.6E+01 | 8.0E+00 | 3.1E+04 |
| IL-21m_Y121E | 2.34 | 1.1E+03 | 2.5E+01 | 2.2E+01 | 3.0E+04 |
| IL-21m_D119N | 2.05 | 1.7E+02 | 1.4E+01 | 6.0E+00 | 3.0E+04 |
| IL-21m_A70F | 2.33 | 1.2E+02 | 1.6E+01 | 1.0E+01 | 2.9E+04 |
| IL-21m_Q95M | 2.41 | 5.0E+02 | 2.8E+01 | 2.0E+01 | 2.9E+04 |
| IL-21m_G105R | 2.06 | 1.1E+02 | 1.1E+01 | 5.0E+00 | 2.9E+04 |
| IL-21m_R132I | 2.21 | 7.0E+02 | 3.1E+01 | 1.8E+01 | 2.8E+04 |
| IL-21m_E40T | 2.13 | 1.7E+02 | 1.5E+01 | 7.0E+00 | 2.8E+04 |
| IL-21m_E131H | 2.22 | 1.4E+02 | 1.4E+01 | 6.0E+00 | 2.8E+04 |
| IL-21m_E131R | 2.25 | 1.1E+02 | 1.3E+01 | 6.0E+00 | 2.7E+04 |
| IL-21m_LA2M | 2 | 3.0E+02 | 1.8E+01 | 1.1E+01 | 2.7E+04 |
| IL-21m_P116N | 2.13 | 5.1E+02 | 2.4E+01 | 1.2E+01 | 2.7E+04 |
| IL-21m_P79G | 2.2 | 2.3E+02 | 1.8E+01 | 9.0E+00 | 2.6E+04 |
| IL-21m_Y45Q | 2.39 | 1.0E+02 | 1.2E+01 | 7.0E+00 | 2.6E+04 |
| IL-21m_L92Y | 2.31 | 3.7E+02 | 2.3E+01 | 2.4E+01 | 2.6E+04 |
| IL-21m_P116G | 2.2 | 3.1E+02 | 2.1E+01 | 1.0E+01 | 2.6E+04 |
| IL-21m_L29M | 2.71 | 1.4E+02 | 1.3E+01 | 7.0E+00 | 2.5E+04 |
| IL-21m_I36R | 2.21 | 1.8E+02 | 1.5E+01 | 1.0E+01 | 2.5E+04 |
| IL-21m_L28Q | 2.07 | 1.7E+02 | 1.8E+01 | 1.2E+01 | 2.5E+04 |
| IL-21m_K123W | 2.05 | 8.5E+02 | 2.7E+01 | 1.6E+01 | 2.5E+04 |
| IL-21m_H144K | 2.11 | 1.3E+02 | 1.4E+01 | 7.0E+00 | 2.5E+04 |
| IL-21m_V60Y | 2.02 | 2.0E+02 | 1.7E+01 | 8.0E+00 | 2.5E+04 |
| IL-21m_L96I | 2.24 | 1.9E+02 | 1.5E+01 | 7.0E+00 | 2.5E+04 |
| IL-21m_P79N | 2.4 | 3.1E+02 | 2.0E+01 | 1.1E+01 | 2.5E+04 |
| IL-21m_L53F | 2.68 | 2.7E+02 | 1.8E+01 | 9.0E+00 | 2.4E+04 |
| IL-21m_E40S | 2.16 | 1.1E+02 | 1.2E+01 | 7.0E+00 | 2.4E+04 |
| IL-21m_A70W | 2.07 | 1.8E+02 | 1.7E+01 | 9.0E+00 | 2.4E+04 |
| IL-21m_L100I | 2.45 | 2.2E+02 | 1.4E+01 | 8.0E+00 | 2.4E+04 |
| IL-21m_I89M | 2.36 | 2.1E+02 | 1.7E+01 | 9.0E+00 | 2.3E+04 |
| IL-21m_Y121S | 2.28 | 3.4E+02 | 2.4E+01 | 1.3E+01 | 2.3E+04 |
| IL-21m_R97Y | 2.04 | 1.9E+03 | 2.4E+01 | 2.1E+01 | 2.3E+04 |
| IL-21m_P79F | 2.12 | 1.4E+02 | 1.3E+01 | 9.0E+00 | 2.3E+04 |
| IL-21m_D119G | 2.26 | 1.2E+02 | 1.4E+01 | 6.0E+00 | 2.3E+04 |
| IL-21m_I38M | 2.12 | 3.2E+02 | 1.5E+01 | 1.1E+01 | 2.3E+04 |
| IL-21m_N85P | 2.1 | 9.8E+02 | 3.2E+01 | 1.8E+01 | 2.2E+04 |
| IL-21m_I141M | 2.06 | 1.9E+02 | 1.4E+01 | 7.0E+00 | 2.2E+04 |
| IL-21m_A94V | 2 | 1.1E+02 | 1.3E+01 | 7.0E+00 | 2.2E+04 |
| IL-21m_G83K | 2.03 | 1.3E+02 | 1.5E+01 | 7.0E+00 | 2.2E+04 |
| IL-21m_W135P | 2.33 | 4.2E+02 | 1.9E+01 | 1.1E+01 | 2.2E+04 |
| IL-21m_P116T | 2.39 | 1.6E+02 | 1.5E+01 | 6.0E+00 | 2.1E+04 |
| IL-21m_V60M | 2.01 | 3.0E+02 | 2.0E+01 | 1.0E+01 | 2.1E+04 |
| IL-21m_V60T | 2.2 | 3.4E+02 | 2.2E+01 | 1.2E+01 | 2.1E+04 |
| IL-21m_S120K | 2.25 | 1.1E+02 | 1.2E+01 | 5.0E+00 | 2.1E+04 |
| IL-21m_P79D | 2.4 | 3.0E+02 | 2.1E+01 | 1.1E+01 | 2.1E+04 |
| IL-21m_E65R | 2.02 | 1.6E+02 | 1.6E+01 | 5.0E+00 | 2.0E+04 |
| IL-21m_S117I | 2.12 | 1.5E+02 | 1.6E+01 | 1.0E+01 | 2.0E+04 |
| IL-21m_L133Y | 2.18 | 1.0E+02 | 1.1E+01 | 6.0E+00 | 2.0E+04 |
| IL-21m_A75L | 2.44 | 4.8E+02 | 2.5E+01 | 1.7E+01 | 1.9E+04 |
| IL-21m_K127W | 2.23 | 1.9E+02 | 1.7E+01 | 9.0E+00 | 1.9E+04 |
| IL-21m_I38A | 2.26 | 1.4E+02 | 1.3E+01 | 7.0E+00 | 1.9E+04 |
| IL-21m_P22R | 2.15 | 7.9E+01 | 1.0E+01 | 6.0E+00 | 1.9E+04 |
| IL-21m_P79W | 2.1 | 2.9E+02 | 1.9E+01 | 9.0E+00 | 1.9E+04 |
| IL-21m_P116S | 2.35 | 1.7E+02 | 1.5E+01 | 9.0E+00 | 1.8E+04 |
| IL-21m_L35I | 2.12 | 1.6E+02 | 1.4E+01 | 9.0E+00 | 1.8E+04 |
| IL-21m_L28R | 2.44 | 7.2E+01 | 1.2E+01 | 5.0E+00 | 1.8E+04 |
| IL-21m_L133A | 2.1 | 1.2E+02 | 1.2E+01 | 7.0E+00 | 1.8E+04 |
| IL-21m_L29V | 2.06 | 1.5E+02 | 1.1E+01 | 6.0E+00 | 1.8E+04 |
| IL-21m_V60P | 2.06 | 2.1E+02 | 1.7E+01 | 9.0E+00 | 1.8E+04 |
| IL-21m_T125I | 2.27 | 1.3E+02 | 1.4E+01 | 8.0E+00 | 1.8E+04 |
| IL-21m_Q41N | 2.14 | 1.2E+02 | 1.2E+01 | 6.0E+00 | 1.8E+04 |
| IL-21m_F72A | 2.27 | 2.8E+02 | 1.8E+01 | 1.0E+01 | 1.7E+04 |
| IL-21m_E122L | 2.01 | 1.6E+02 | 1.5E+01 | 8.0E+00 | 1.7E+04 |
| IL-21m_K43D | 2.6 | 3.8E+02 | 2.3E+01 | 1.4E+01 | 1.7E+04 |
| IL-21m_E122H | 2.37 | 1.3E+02 | 1.1E+01 | 6.0E+00 | 1.7E+04 |
| IL-21m_F129A | 2.18 | 2.1E+02 | 1.7E+01 | 8.0E+00 | 1.6E+04 |
| IL-21m_E131A | 2.15 | 7.3E+01 | 1.0E+01 | 5.0E+00 | 1.6E+04 |
| IL-21m_A56I | 2.05 | 2.2E+02 | 1.7E+01 | 9.0E+00 | 1.6E+04 |
| IL-21m_P79Q | 2.14 | 2.4E+02 | 2.2E+01 | 1.4E+01 | 1.6E+04 |
| IL-21m_L92F | 2.37 | 3.5E+02 | 1.8E+01 | 1.0E+01 | 1.6E+04 |
| IL-21m_A67P | 2.08 | 1.4E+02 | 1.5E+01 | 9.0E+00 | 1.5E+04 |
| IL-21m_L29T | 2.18 | 1.3E+02 | 1.1E+01 | 7.0E+00 | 1.5E+04 |
| IL-21m_Q95N | 2.42 | 2.1E+02 | 1.6E+01 | 1.1E+01 | 1.5E+04 |
| IL-21m_I36F | 2.07 | 1.2E+02 | 1.2E+01 | 7.0E+00 | 1.5E+04 |
| IL-21m_L53I | 2.14 | 1.2E+02 | 1.1E+01 | 6.0E+00 | 1.5E+04 |
| IL-21m_E131G | 2.39 | 1.4E+02 | 1.3E+01 | 6.0E+00 | 1.5E+04 |
| IL-21m_F69V | 2.35 | 1.4E+02 | 1.3E+01 | 7.0E+00 | 1.4E+04 |
| IL-21m_Q138K | 2.31 | 1.0E+02 | 1.5E+01 | 7.0E+00 | 1.4E+04 |
| IL-21m_I36W | 2.04 | 1.9E+02 | 1.6E+01 | 8.0E+00 | 1.4E+04 |
| IL-21m_S55K | 2.19 | 9.5E+01 | 1.2E+01 | 5.0E+00 | 1.4E+04 |
| IL-21m_L77M | 2.26 | 2.8E+02 | 1.9E+01 | 1.0E+01 | 1.4E+04 |
| IL-21m_I90H | 2.16 | 7.8E+01 | 1.0E+01 | 4.0E+00 | 1.4E+04 |
| IL-21m_D59Q | 2.52 | 1.9E+02 | 1.5E+01 | 8.0E+00 | 1.3E+04 |
| IL-21m_H144W | 2.29 | 4.2E+02 | 2.0E+01 | 1.5E+01 | 1.3E+04 |
| IL-21m_K123T | 2.02 | 2.6E+02 | 2.3E+01 | 1.5E+01 | 1.3E+04 |
| IL-21m_L29A | 2.42 | 1.3E+02 | 1.3E+01 | 7.0E+00 | 1.3E+04 |
| IL-21m_L145G | 2.24 | 9.6E+01 | 1.2E+01 | 6.0E+00 | 1.3E+04 |
| IL-21m_P126A | 2.17 | 2.7E+02 | 1.9E+01 | 1.1E+01 | 1.3E+04 |
| IL-21m_S120G | 2.03 | 1.6E+02 | 1.6E+01 | 8.0E+00 | 1.3E+04 |
| IL-21m_F88D | 2.43 | 1.3E+02 | 1.0E+01 | 8.0E+00 | 1.3E+04 |
| IL-21m_E128L | 2.27 | 1.4E+02 | 1.4E+01 | 7.0E+00 | 1.3E+04 |
| IL-21m_L145D | 2.14 | 2.1E+02 | 1.8E+01 | 9.0E+00 | 1.3E+04 |
| IL-21m_I36N | 2.38 | 1.1E+02 | 1.2E+01 | 7.0E+00 | 1.2E+04 |
| IL-21m_E122M | 2.1 | 1.3E+02 | 1.4E+01 | 7.0E+00 | 1.2E+04 |
| IL-21m_L145W | 2.41 | 1.3E+02 | 1.2E+01 | 6.0E+00 | 1.2E+04 |
| IL-21m_A94H | 2.38 | 5.5E+01 | 9.0E+00 | 3.0E+00 | 1.2E+04 |
| IL-21m_I90K | 2.25 | 6.0E+01 | 9.0E+00 | 3.0E+00 | 1.2E+04 |
| IL-21m_P116L | 2.18 | 1.8E+02 | 1.6E+01 | 1.1E+01 | 1.1E+04 |
| IL-21m_L145R | 2.21 | 1.4E+02 | 1.3E+01 | 7.0E+00 | 1.1E+04 |
| IL-21m_Q138R | 2.42 | 8.7E+01 | 1.1E+01 | 6.0E+00 | 1.1E+04 |
| IL-21m_Y121G | 2.48 | 3.1E+02 | 2.2E+01 | 1.6E+01 | 1.1E+04 |
| IL-21m_E131F | 2.35 | 1.1E+02 | 1.2E+01 | 5.0E+00 | 1.1E+04 |
| IL-21m_A70H | 2.08 | 1.0E+02 | 1.2E+01 | 5.0E+00 | 1.1E+04 |
| IL-21m_E122Q | 2.13 | 6.4E+01 | 1.1E+01 | 4.0E+00 | 1.1E+04 |
| IL-21m_L145F | 2.1 | 1.1E+02 | 1.0E+01 | 6.0E+00 | 1.1E+04 |
| IL-21m_G105K | 2.12 | 1.1E+02 | 1.3E+01 | 5.0E+00 | 1.0E+04 |
| IL-21m_L136I | 2.11 | 1.1E+02 | 1.2E+01 | 7.0E+00 | 1.0E+04 |
| IL-21m_E128Q | 2.25 | 1.4E+02 | 1.5E+01 | 8.0E+00 | 9.8E+03 |
| IL-21m_I141Y | 2.14 | 1.2E+02 | 1.1E+01 | 6.0E+00 | 9.6E+03 |
| IL-21m_L136V | 2.41 | 1.0E+02 | 1.1E+01 | 5.0E+00 | 9.6E+03 |
| IL-21m_L29G | 2.2 | 1.4E+02 | 1.3E+01 | 8.0E+00 | 9.5E+03 |
| IL-21m_P51R | 2.17 | 6.1E+01 | 9.0E+00 | 3.0E+00 | 9.3E+03 |
| IL-21m_Q95A | 2.1 | 9.5E+01 | 1.1E+01 | 6.0E+00 | 9.2E+03 |
| IL-21m_E40G | 2.36 | 9.3E+01 | 1.2E+01 | 7.0E+00 | 9.1E+03 |
| IL-21m_Y45R | 2.27 | 1.1E+02 | 1.2E+01 | 7.0E+00 | 9.0E+03 |
| IL-21m_T125K | 2.24 | 9.9E+01 | 1.2E+01 | 5.0E+00 | 8.6E+03 |
| IL-21m_L29E | 2.03 | 2.0E+02 | 1.5E+01 | 1.0E+01 | 8.5E+03 |
| IL-21m_V60W | 2 | 1.7E+02 | 1.4E+01 | 7.0E+00 | 8.4E+03 |
| IL-21m_E46N | 2.08 | 1.2E+02 | 1.2E+01 | 6.0E+00 | 8.2E+03 |
| IL-21m_F69M | 2.36 | 1.1E+02 | 1.1E+01 | 5.0E+00 | 8.2E+03 |
| IL-21m_Y45A | 2.43 | 1.4E+02 | 1.2E+01 | 7.0E+00 | 8.2E+03 |
| IL-21m_R99L | 2.13 | 3.9E+02 | 1.7E+01 | 8.0E+00 | 7.9E+03 |
| IL-21m_F88A | 2.21 | 1.2E+02 | 1.0E+01 | 5.0E+00 | 7.9E+03 |
| IL-21m_L54A | 2.14 | 1.7E+02 | 1.3E+01 | 7.0E+00 | 7.8E+03 |
| IL-21m_E122G | 2.31 | 7.0E+01 | 1.0E+01 | 5.0E+00 | 7.7E+03 |
| IL-21m_L145S | 2.11 | 1.3E+02 | 1.5E+01 | 7.0E+00 | 7.4E+03 |
| IL-21m_M140L | 2.35 | 1.7E+02 | 1.5E+01 | 8.0E+00 | 7.3E+03 |
| IL-21m_R132T | 2.1 | 2.0E+02 | 1.9E+01 | 1.0E+01 | 7.3E+03 |
| IL-21m_E131S | 2.13 | 7.3E+01 | 1.0E+01 | 5.0E+00 | 7.3E+03 |
| IL-21m_K123H | 2.02 | 1.4E+02 | 1.7E+01 | 9.0E+00 | 7.1E+03 |
| IL-21m_E122N | 2.15 | 1.1E+02 | 1.3E+01 | 6.0E+00 | 7.0E+03 |
| IL-21m_F88Q | 2.2 | 8.5E+01 | 8.0E+00 | 4.0E+00 | 6.9E+03 |
| IL-21m_G106K | 2.36 | 7.6E+01 | 1.0E+01 | 4.0E+00 | 6.6E+03 |
| IL-21m_E122S | 2.08 | 7.5E+01 | 1.1E+01 | 6.0E+00 | 6.6E+03 |
| IL-21m_E131M | 2.23 | 7.7E+01 | 9.0E+00 | 5.0E+00 | 6.5E+03 |
| IL-21m_I36D | 2.6 | 1.6E+02 | 1.5E+01 | 9.0E+00 | 6.3E+03 |
| IL-21m_L145A | 2.3 | 5.7E+01 | 9.0E+00 | 4.0E+00 | 6.3E+03 |
| IL-21m_F88M | 2.28 | 6.5E+01 | 8.0E+00 | 4.0E+00 | 6.3E+03 |
| IL-21m_Q109K | 2.35 | 6.2E+01 | 9.0E+00 | 3.0E+00 | 6.3E+03 |
| IL-21m_L54S | 2.29 | 1.5E+02 | 1.4E+01 | 8.0E+00 | 6.2E+03 |
| IL-21m_G83P | 2.29 | 5.6E+01 | 9.0E+00 | 5.0E+00 | 6.0E+03 |
| IL-21m_P116K | 2.44 | 8.7E+01 | 1.2E+01 | 6.0E+00 | 6.0E+03 |
| IL-21m_T87H | 2.26 | 4.4E+01 | 7.0E+00 | 3.0E+00 | 5.9E+03 |
| IL-21m_R132K | 2.02 | 1.2E+02 | 1.3E+01 | 7.0E+00 | 5.9E+03 |
| IL-21m_L145H | 2.29 | 1.3E+02 | 1.2E+01 | 5.0E+00 | 5.6E+03 |
| IL-21m_A67Y | 2.25 | 1.7E+02 | 1.5E+01 | 9.0E+00 | 5.4E+03 |
| IL-21m_Q23R | 2 | 5.9E+01 | 9.0E+00 | 4.0E+00 | 5.4E+03 |
| IL-21m_G105H | 2.06 | 6.8E+01 | 9.0E+00 | 3.0E+00 | 5.4E+03 |
| IL-21m_S120R | 2.26 | 1.0E+02 | 1.2E+01 | 6.0E+00 | 5.3E+03 |
| IL-21m_V39A | 2.34 | 8.0E+01 | 1.0E+01 | 6.0E+00 | 5.3E+03 |
| IL-21m_A75M | 2.28 | 1.5E+02 | 1.3E+01 | 8.0E+00 | 5.2E+03 |
| IL-21m_I112R | 2.24 | 1.2E+02 | 1.4E+01 | 5.0E+00 | 5.2E+03 |
| IL-21m_P116M | 2.4 | 1.1E+02 | 1.2E+01 | 7.0E+00 | 5.2E+03 |
| IL-21m_P126T | 2.09 | 1.4E+02 | 1.3E+01 | 7.0E+00 | 5.2E+03 |
| IL-21m_L28V | 2.03 | 8.3E+01 | 1.3E+01 | 7.0E+00 | 5.1E+03 |
| IL-21m_Q138Y | 2.18 | 6.2E+01 | 9.0E+00 | 5.0E+00 | 5.1E+03 |
| IL-21m_A94G | 2.21 | 9.9E+01 | 1.3E+01 | 9.0E+00 | 5.0E+03 |
| IL-21m_F88E | 2.18 | 7.9E+01 | 9.0E+00 | 6.0E+00 | 5.0E+03 |
| IL-21m_L54H | 2.14 | 7.8E+01 | 9.0E+00 | 4.0E+00 | 4.9E+03 |
| IL-21m_H144D | 2.1 | 9.0E+01 | 1.1E+01 | 1.1E+01 | 4.9E+03 |
| IL-21m_L77V | 2.29 | 8.2E+01 | 1.2E+01 | 6.0E+00 | 4.8E+03 |
| IL-21m_L136H | 2.42 | 6.1E+01 | 8.0E+00 | 4.0E+00 | 4.8E+03 |
| IL-21m_Q95S | 2.25 | 1.9E+02 | 1.7E+01 | 8.0E+00 | 4.7E+03 |
| IL-21m_N84W | 2.03 | 1.4E+02 | 1.4E+01 | 8.0E+00 | 4.7E+03 |
| IL-21m_L145Y | 2.12 | 6.6E+01 | 1.1E+01 | 5.0E+00 | 4.7E+03 |
| IL-21m_L28T | 2.16 | 6.7E+01 | 9.0E+00 | 5.0E+00 | 4.6E+03 |
| IL-21m_K123Y | 2.01 | 1.1E+02 | 1.3E+01 | 7.0E+00 | 4.6E+03 |
| IL-21m_R132H | 2.26 | 1.0E+02 | 1.2E+01 | 7.0E+00 | 4.5E+03 |
| IL-21m_R99P | 2 | 3.2E+02 | 1.1E+01 | 1.2E+01 | 4.4E+03 |
| IL-21m_L32F | 2.3 | 9.5E+01 | 1.2E+01 | 6.0E+00 | 4.3E+03 |
| IL-21m_S117V | 2.17 | 8.5E+01 | 1.2E+01 | 7.0E+00 | 4.3E+03 |
| IL-21m_E122V | 2.26 | 7.1E+01 | 1.1E+01 | 5.0E+00 | 4.2E+03 |
| IL-21m_A75V | 2.28 | 1.4E+02 | 1.3E+01 | 7.0E+00 | 4.2E+03 |
| IL-21m_F88N | 2.32 | 8.8E+01 | 1.0E+01 | 6.0E+00 | 4.2E+03 |
| IL-21m_L145K | 2.4 | 5.7E+01 | 9.0E+00 | 4.0E+00 | 4.2E+03 |
| IL-21m_E52T | 2.01 | 3.5E+01 | 7.0E+00 | 3.0E+00 | 4.1E+03 |
| IL-21m_K123P | 2.18 | 1.6E+02 | 1.5E+01 | 1.7E+01 | 4.1E+03 |
| IL-21m_L136F | 2.25 | 9.6E+01 | 1.1E+01 | 5.0E+00 | 4.0E+03 |
| IL-21m_A67N | 2.04 | 8.0E+01 | 1.1E+01 | 5.0E+00 | 4.0E+03 |
| IL-21m_L53H | 2.1 | 7.0E+01 | 9.0E+00 | 4.0E+00 | 3.9E+03 |
| IL-21m_E40A | 2.15 | 7.2E+01 | 1.1E+01 | 5.0E+00 | 3.8E+03 |
| IL-21m_T125V | 2.31 | 6.5E+01 | 1.1E+01 | 6.0E+00 | 3.8E+03 |
| IL-21m_H144R | 2.13 | 9.1E+01 | 1.2E+01 | 6.0E+00 | 3.8E+03 |
| IL-21m_G83H | 2.16 | 6.1E+01 | 1.0E+01 | 4.0E+00 | 3.8E+03 |
| IL-21m_G106H | 2 | 5.3E+01 | 8.0E+00 | 3.0E+00 | 3.8E+03 |
| IL-21m_E46H | 2.08 | 6.0E+01 | 8.0E+00 | 4.0E+00 | 3.8E+03 |
| IL-21m_R132G | 2.09 | 1.1E+02 | 1.2E+01 | 7.0E+00 | 3.8E+03 |
| IL-21m_R99M | 2.03 | 3.7E+02 | 1.6E+01 | 1.4E+01 | 3.8E+03 |
| IL-21m_S80T | 2.26 | 1.1E+02 | 1.1E+01 | 6.0E+00 | 3.7E+03 |
| IL-21m_P116R | 2.21 | 9.6E+01 | 1.1E+01 | 5.0E+00 | 3.7E+03 |
| IL-21m_G83Y | 2.03 | 9.6E+01 | 1.1E+01 | 7.0E+00 | 3.6E+03 |
| IL-21m_S80H | 2.02 | 5.5E+01 | 9.0E+00 | 4.0E+00 | 3.6E+03 |
| IL-21m_F88W | 2.16 | 8.0E+01 | 1.1E+01 | 6.0E+00 | 3.6E+03 |
| IL-21m_R99H | 2.19 | 8.5E+01 | 1.0E+01 | 7.0E+00 | 3.5E+03 |
| IL-21m_E131V | 2.13 | 6.4E+01 | 1.0E+01 | 5.0E+00 | 3.5E+03 |
| IL-21m_A113H | 2.23 | 7.4E+01 | 1.0E+01 | 4.0E+00 | 3.5E+03 |
| IL-21m_E122A | 2.13 | 5.5E+01 | 9.0E+00 | 4.0E+00 | 3.5E+03 |
| IL-21m_Q109H | 2.17 | 7.9E+01 | 1.1E+01 | 4.0E+00 | 3.4E+03 |
| IL-21m_I141L | 2.16 | 7.1E+01 | 9.0E+00 | 5.0E+00 | 3.4E+03 |
| IL-21m_Q138F | 2.09 | 5.9E+01 | 1.0E+01 | 5.0E+00 | 3.4E+03 |
| IL-21m_P79M | 2.16 | 7.1E+01 | 1.0E+01 | 5.0E+00 | 3.4E+03 |
| IL-21m_A70G | 2.28 | 8.0E+01 | 1.1E+01 | 6.0E+00 | 3.3E+03 |
| IL-21m_E122I | 2.47 | 6.8E+01 | 1.1E+01 | 5.0E+00 | 3.3E+03 |
| IL-21m_K123V | 2.17 | 1.8E+02 | 1.6E+01 | 9.0E+00 | 3.3E+03 |
| IL-21m_H111K | 2.15 | 6.4E+01 | 1.2E+01 | 6.0E+00 | 3.3E+03 |
| IL-21m_G83W | 2.23 | 8.1E+01 | 1.1E+01 | 6.0E+00 | 3.3E+03 |
| IL-21m_I36Y | 2.02 | 8.6E+01 | 1.0E+01 | 6.0E+00 | 3.2E+03 |
| IL-21m_N84Q | 2.02 | 4.1E+01 | 8.0E+00 | 3.0E+00 | 3.2E+03 |
| IL-21m_I36S | 2.58 | 7.2E+01 | 9.0E+00 | 5.0E+00 | 3.2E+03 |
| IL-21m_E46G | 2 | 1.0E+02 | 1.2E+01 | 7.0E+00 | 3.2E+03 |
| IL-21m_E122T | 2.03 | 6.7E+01 | 1.1E+01 | 5.0E+00 | 3.2E+03 |
| IL-21m_R124H | 2.05 | 7.5E+01 | 1.1E+01 | 6.0E+00 | 3.1E+03 |
| IL-21m_N81W | 2.04 | 6.9E+01 | 1.0E+01 | 6.0E+00 | 3.1E+03 |
| IL-21m_A70K | 2.21 | 5.1E+01 | 1.0E+01 | 3.0E+00 | 3.1E+03 |
| IL-21m_T125R | 2.29 | 4.1E+01 | 9.0E+00 | 4.0E+00 | 3.1E+03 |
| IL-21m_S117L | 2.07 | 7.4E+01 | 1.0E+01 | 5.0E+00 | 3.1E+03 |
| IL-21m_L29Y | 2.11 | 7.1E+01 | 9.0E+00 | 5.0E+00 | 3.1E+03 |
| IL-21m_I141E | 2.22 | 1.0E+02 | 1.2E+01 | 6.0E+00 | 3.0E+03 |
| IL-21m_N84Y | 2.06 | 7.0E+01 | 9.0E+00 | 5.0E+00 | 3.0E+03 |
| IL-21m_R132E | 2.05 | 1.4E+02 | 1.5E+01 | 8.0E+00 | 3.0E+03 |
| IL-21m_Q143K | 2.35 | 6.3E+01 | 9.0E+00 | 4.0E+00 | 3.0E+03 |
| IL-21m_R132D | 2.13 | 1.3E+02 | 1.6E+01 | 9.0E+00 | 3.0E+03 |
| IL-21m_K86V | 2.24 | 7.3E+01 | 1.1E+01 | 6.0E+00 | 2.9E+03 |
| IL-21m_R99G | 2.29 | 4.5E+02 | 1.2E+01 | 1.0E+01 | 2.8E+03 |
| IL-21m_K110R | 2.02 | 9.2E+01 | 1.2E+01 | 5.0E+00 | 2.8E+03 |
| IL-21m_R97V | 2.15 | 1.1E+03 | 2.6E+01 | 1.7E+01 | 2.8E+03 |
| IL-21m_V93T | 2.32 | 6.9E+01 | 1.0E+01 | 4.0E+00 | 2.8E+03 |
| IL-21m_I36A | 2.55 | 5.9E+01 | 8.0E+00 | 4.0E+00 | 2.8E+03 |
| IL-21m_G62K | 2.52 | 6.1E+01 | 1.0E+01 | 4.0E+00 | 2.8E+03 |
| IL-21m_S55G | 2.07 | 8.5E+01 | 1.1E+01 | 6.0E+00 | 2.8E+03 |
| IL-21m_S80D | 2.02 | 1.1E+02 | 1.2E+01 | 7.0E+00 | 2.7E+03 |
| IL-21m_Y121A | 2.21 | 9.8E+01 | 1.0E+01 | 6.0E+00 | 2.7E+03 |
| IL-21m_L96M | 2.14 | 8.4E+01 | 1.1E+01 | 5.0E+00 | 2.7E+03 |
| IL-21m_Q138S | 2.33 | 7.1E+01 | 9.0E+00 | 5.0E+00 | 2.7E+03 |
| IL-21m_L137I | 2.2 | 5.3E+01 | 9.0E+00 | 4.0E+00 | 2.6E+03 |
| IL-21m_K123L | 2.23 | 2.1E+02 | 1.5E+01 | 9.0E+00 | 2.6E+03 |
| IL-21m_L133M | 2.14 | 5.5E+01 | 9.0E+00 | 5.0E+00 | 2.6E+03 |
| IL-21m_P79T | 2.03 | 7.5E+01 | 1.1E+01 | 6.0E+00 | 2.6E+03 |
| IL-21m_S120A | 2.06 | 4.7E+01 | 9.0E+00 | 4.0E+00 | 2.6E+03 |
| IL-21m_S117N | 2.16 | 8.0E+01 | 1.4E+01 | 7.0E+00 | 2.6E+03 |
| IL-21m_S80E | 2.17 | 9.1E+01 | 1.2E+01 | 6.0E+00 | 2.6E+03 |
| IL-21m_Y121F | 2.17 | 6.8E+01 | 1.1E+01 | 5.0E+00 | 2.6E+03 |
| IL-21m_E65D | 2.05 | 6.4E+01 | 1.0E+01 | 5.0E+00 | 2.5E+03 |
| IL-21m_Y45N | 2.51 | 6.8E+01 | 1.0E+01 | 6.0E+00 | 2.5E+03 |
| IL-21m_F72Y | 2.27 | 5.7E+01 | 1.0E+01 | 5.0E+00 | 2.5E+03 |
| IL-21m_H144N | 2.23 | 8.6E+01 | 1.2E+01 | 6.0E+00 | 2.5E+03 |
| IL-21m_T125Y | 2.04 | 5.1E+01 | 9.0E+00 | 4.0E+00 | 2.5E+03 |
| IL-21m_D59S | 2.09 | 6.1E+01 | 1.0E+01 | 5.0E+00 | 2.5E+03 |
| IL-21m_E128D | 2.21 | 6.5E+01 | 9.0E+00 | 5.0E+00 | 2.5E+03 |
| IL-21m_L145Q | 2.21 | 6.0E+01 | 9.0E+00 | 4.0E+00 | 2.5E+03 |
| IL-21m_Q143W | 2.53 | 1.3E+02 | 1.5E+01 | 6.0E+00 | 2.4E+03 |
| IL-21m_A75E | 2.1 | 1.5E+02 | 1.4E+01 | 9.0E+00 | 2.2E+03 |
| IL-21m_Q143F | 2.28 | 1.1E+02 | 1.1E+01 | 6.0E+00 | 2.2E+03 |
| IL-21m_P79E | 2.44 | 1.4E+02 | 1.3E+01 | 6.0E+00 | 2.0E+03 |
| IL-21m_H144Y | 2.09 | 1.1E+02 | 1.1E+01 | 7.0E+00 | 1.9E+03 |
| IL-21m_L54E | 2.32 | 1.6E+02 | 1.3E+01 | 8.0E+00 | 1.6E+03 |
| IL-21m_R99I | 2.25 | 2.3E+02 | 1.8E+01 | 1.2E+01 | 1.4E+03 |
| IL-21m_K123Q | 2.11 | 9.4E+01 | 1.5E+01 | 1.0E+01 | 1.4E+03 |
| IL-21m_K76W | 2.11 | 1.4E+02 | 1.4E+01 | 8.0E+00 | 1.4E+03 |
| IL-21m_R99N | 2.31 | 2.1E+02 | 1.3E+01 | 8.0E+00 | 1.2E+03 |
| IL-21m_S55D | 2.13 | 1.1E+02 | 1.4E+01 | 8.0E+00 | 1.2E+03 |
| IL-21m_R99D | 2.25 | 3.1E+03 | 2.8E+01 | 3.1E+01 | 1.1E+03 |
| IL-21m_K78D | 2.38 | 2.7E+02 | 2.1E+01 | 1.1E+01 | 1.0E+03 |
| IL-21m_R97M | 2.16 | 1.6E+02 | 1.2E+01 | 7.0E+00 | 7.7E+02 |
| IL-21m_R97T | 2.39 | 1.1E+02 | 1.1E+01 | 7.0E+00 | 6.1E+02 |
| IL-21m_R99V | 2.54 | 1.2E+02 | 1.1E+01 | 7.0E+00 | 4.4E+02 |

In some embodiments, the human IL-21 variant includes amino acid substitutions having one or more specific substitutions at one or more, e.g., two or more, three or more, four or more, five or more, six or more, seven or more, eight or more, nine or more, or ten or more, of positions Q32, D33, R34, H35, M36, I37, R38, M39, Q41, L42, I43, D44, I45, V46, D47, Q48, L49, N51, Y52, V53, N54, D55, L56, V57, P58, E59, F60, L61, P62, A63, E65, D66, V67, E68, N70, E72, W73, S74, A75, F76, S77, F79, Q80, K81, A82, Q83, L84, K85, S86, A87, N88, T89, G90, N92, E93, R94, I95, I96, N97, V98, S99, K101, I100, K102, L103, K104, R105, K106, P107, P108, S109, T110, N111, A112, G113, R114, R115, H118, L120, K131, P132, P133, R139, S142, L144, Q145, I148, Q150, H151, and L152 in the wild-type human IL-21 amino acid sequence (SEQ ID NO: 1).

In some embodiments, the IL-21 variant includes amino acid substitutions having one or more, e.g., two or more, or three or more, or four or more, or five or more, or six or more, or seven or more, or eight or more, or nine or more, or ten or more specific substitutions selected from the group consisting of Q32K, Q32I, D33H, D33A, D33N, D33P, D33Q, D33R, D33S, D33T, D33W, D33V, R34A, R34D, R34E, R34F, R34G, R34H, R34K, R34L, R34M, R34N, R34P, R34Q, R34S, R34T, R34V, R34W, R34Y, H35P, H35I, H35F, M36T, M36R, M36P, I37E, I37G, I37K, I37N, I37Q, I37R, I37S, I37V, I37P, I37A, I37W, I37H, I37D, I37T, I37F, R38A, R38P, R38E, R38F, R38K, R38M, R38Q, R38V, R38W, R38N, R38G, R38L, R38S, R38T, R38I, R38D, M39D, M39E, M39G, M39I, M39K, M39P, M39Q, M39R, M39A, Q41K, Q41P, Q41S, Q41E, Q41D, Q41W, Q41R, L42D, L42E, L42G, L42H, L42K, L42N, L42Q, L42R, L42S, L42Y, I43D, I43L, I43M, I43P, I43S, I43Y, I43F, I43R, D44A, D44G, D44N, D44P, D44T, D44W, D44Y, D44F, D44Q, D44S, D44V, D44M, D44L, D44P, D44H, D44R, D44K, I45A, I45D, I45G, I45H, I45N, I45P, I45R, I45S, I45Y, I45T, I45Q, I45F, I45W, I45K, V46D, V46E, V46G, V46K, V46N, V46P, V46Q, V46T, D47P, D47S, D47R, D47K, D47F, D47G, Q48G, Q48K, Q48R, L49D, L49E, L49H, L49K, L49N, L49P, L49R, L49S, L49F, N51E, N51G, N51P, N51V, Y52D, Y52H, Y52K, Y52N, Y52Q, Y52S, Y52T, Y52R, Y52G, V53A, V53E, V53H, V53N, V53P, V53Q, V53S, V53T, V53M, N54H, N54K, N54P, D55K, D55R, D55Y, D55M, L56E, L56F, L56M, L56N, L56S, L56T, L56V, L56D, V57H, V57L, V57M, V57P, V57R, V57T, V57Y, P58M, P58K, E59R, E59S, E59V, E59Y, F60H, F60I, F60R, L61H, L61I, L61K, P62H, P62S, P62T, P62V, P62L, A63T, E65R, D66M, D66R, D66K, V67L, V67K, V67G, E68K, E68R, E68G, N70A, N70H, N70G, E72H, E72I, E72L, E72S, E72T, E72R, E72W, W73A, W73G, W73K, W73L, W73N, W73P, W73Q, W73S, W73T, W73V, W73Y, S74A, S74H, S74N, S74R, S74K, A75N, A75Y, A75V, F76D, F76E, F76H, F76M, F76P, F76Q, F76R, F76T, F76S, F76L, F76V, F76I, F76K, S77K, S77M, S77R, S77W, F79D, F79E, F79K, F79P, F79R, F79T, F79Y, F79I, F79A, F79L, F79V, Q80A, Q80D, Q80L, Q80M, Q80V, Q80I, Q80E, Q80N, Q80H, Q80W, Q80F, Q80K, Q80R, Q80G, Q80Y, K81D, K81H, A82V, A82W, A82M, A82K, A82L, A82I, A82H, A82T, A82Y, A82V, Q83L, Q83W, Q83F, Q83K, Q83H, Q83V, L84P, L84F, L84T, K85S, K85P, S86I, S86M, S86L, S86R, A87N, A87P, N88K, N88R, T89L, T89F, G90Y, G90Q, G90I, G90K, N92G, N92Y, N92W, N92P, E93G, E93T, E93R, E93F, E93P, E93K, E93S, E93V, E93I, E93Y, R94S, R94T, I95H, I95K, I95P, I95T, I95R, I95G, I96A, I96D, I96G, I96P, I96S, I96T, I96V, I96F, I96Y, I96E, I96Q, I96M, I96H, I96N, I96R, I96K, N97G, N97P, N97H, N97W, N97K, N97R, V98H, V98K, V98R, V98E, V98A, V98G, V98Y, V98N, V98S, V98M, V98T, V98P, V98F, S99H, S99R, S99P, S99K, S99G, I100E, I100R, I100S, HOOD, I100G, I100F, I100Y, I100Q, I100H, I100A, I100R, K101E, K101D, K102P, K102G, K102L, K102V, K102E, K102I, K102T, K102D, L103E, L103A, L103S, L103T, L103F, L103I, L103W, L103Y, L103V, L103Q, L103H, L103N, K104G, K104P, R105A, R105D, R105K, R105Q, R105S, R105W, R105Y, R105L, R105H, R105M, R105F, R105N, R105I, R105V, R105T, R105E, R105G, R105P, K106E, K106N, K106P, K106S, K106T, K106G, K106H, K106D, P107D, P107Y, P107V, P107H, P107V, P107K, P107R, P108D, P108E, P108G, P108I, P108L, P108Q, P108T, P108A, P108H, P108K, P108Y, P108M, P108N, P108W, P108R, S109K, S109M, S109R, T110D, T110S, T110H, T110N, N111R, N111K, A112P, A112R, A112K, G113R, G113H, R114F, R114Q, R115I, R115P, R115Q, R115D, H118I, H118K, H118R, L120H, L120K, L120M, L120V, L120Y, L120S, K131M, K131V, P132A, P133I, R139S, S142D, S142H, S142I, L144M, Q145H, I148S, Q150D, Q150R, Q150S, H151E, and L152H in the wild-type human IL-21 amino acid sequence (SEQ ID NO: 1).

In some embodiments, the human IL-21 variant includes amino acid substitutions having one or more specific substitutions at one or more, e.g., two or more, three or more, four or more, five or more, six or more, seven or more, eight or more, nine or more, of positions H35, N91, K101, N111, R114, R115, H118, R119, T121 in the wild-type human IL-21 amino acid sequence (SEQ ID NO: 1).

In some embodiments, the IL-21 variant includes amino acid substitutions having one or more, e.g., two or more, or three or more, or four or more, or five or more, or six or more, or seven or more, or eight or more, or nine or more specific substitutions selected from the group consisting of H35F, N91E, K101E, N111E, R114G, R115G, H118D, R119T, and T121E in the wild-type human IL-21 amino acid sequence (SEQ ID NO: 1).

In some embodiments, the IL-21 variant includes the amino acid substitutions H35F, N91E, K101E, N111E, R114G, R115G, H118D, R119T, and T121E in the wild-type human IL-21 amino acid sequence (SEQ ID NO: 1). In some embodiments, the IL-21 variant includes the amino acid substitutions H35F, N91E, and K101E in the wild-type human IL-21 amino acid sequence (SEQ ID NO: 1). In some embodiments, the IL-21 variant includes the amino acid substitutions N111E, R114G, R115G, H118D, R119T, and T121E in the wild-type human IL-21 amino acid sequence (SEQ ID NO: 1). In some embodiments, the IL-21 variant includes the amino acid substitutions N111E, R115G, H118D, and T121E in the wild-type human IL-21 amino acid sequence (SEQ ID NO: 1). In some embodiments, the IL-21 variant includes the amino acid substitutions H35F, R114G, R115G, H118D, R119T, and T121E in the wild-type human IL-21 amino acid sequence (SEQ ID NO: 1).

In some embodiments, the human IL-21 variant includes amino acid substitutions having one or more specific substitutions at one or more, e.g., two or more, three or more, four or more, five or more, six or more, seven or more, eight or more, nine or more, ten or more, 11 or more, 12 or more, 13 of positions H35, I45, N70, K81, V98, P107, T110, A112, R114, R115, H118, R119, T121 in the wild-type human IL-21 amino acid sequence (SEQ ID NO: 1).

In some embodiments, the IL-21 variant includes amino acid substitutions having one or more, e.g., two or more, or three or more, or four or more, or five or more, or six or more, or seven or more, or eight or more, or nine or more, ten or more, 11 or more, 12 or more, 13 or more, 14 or more, 15 or more, or 16 specific substitutions selected from the group consisting of H35F, H35I, I45T, N70G, K81D, V98E, P107V, T11D, A112P, R114Q, R114G, R115D, R115G, H118D, R119T, and T121E in the wild-type human IL-21 amino acid sequence (SEQ ID NO: 1).

In some embodiments, the human IL-21 variant has a binding affinity to the human IL-21R that is decreased by 1.5-fold, 2-fold, 3-fold, 5-fold, 10-fold, 50-fold, 100-fold, 500-fold, 1000-fold, 2000-fold, or 3000-fold, or more relative to the binding affinity between the wild-type human IL-21 polypeptide and the human IL-21R. In some embodiments, the mouse IL-21 variant has a binding affinity to the mouse IL-21R that is decreased by 1.5-fold, 2-fold, 3-fold, 5-fold, 10-fold, 50-fold, 100-fold, 500-fold, 1000-fold, 2000-fold, or 3000-fold or more relative to the binding affinity between the wild-type mouse IL-21 polypeptide and the mouse IL-21R.

### IL-21 Fusion Proteins and Methods of Making

This disclosure provides isolated IL-21 fusion proteins (e.g., antibody-IL-21 fusion proteins) that have decreased or no binding to the IL-21R. Such IL-21 fusion proteins can deliver cytokines to a desired cell type while minimizing peripheral exposure and thus reducing overall potential adverse effects, e.g., adverse systemic effects.

The isolated fusion proteins include: an antibody comprising an Fc domain; and a human IL-21 variant, wherein the IL-21 variant is covalently linked to the Fc domain of the antibody.

In some embodiments, one or more polypeptides (e.g., heterologous or homologous sequence) can be inserted between the antibody and the IL-21 variant of the IL-21 fusion proteins as described herein. In some embodiments, the polypeptide can be inserted or conjugated at the amino terminus, at the carboxyl terminus, or both the amino and carboxyl termini of the antibody or domain thereof. In some embodiments, the polypeptide includes a polypeptide linker conjugating the antibody and the IL-21 variant. In some embodiments, the polypeptide comprises one or more linker(s) and tag(s). Examples of a polypeptide tag include, but not are not limited to, a FLAG tag, a 6His tag, an 8His tag, or an AVI tag.

The antibodies useful in the IL-21 fusion proteins of the present invention can encompass monoclonal antibodies, polyclonal antibodies, antibody fragments (e.g., Fab, Fab′, F(ab′)2, Fv, Fe, etc.), chimeric antibodies, bispecific antibodies, heteroconjugate antibodies, single chain (ScFv), mutants thereof, fusion proteins comprising an antibody portion (e.g., a domain antibody), humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies.

In some embodiments, an antibody constant region can be modified to avoid interaction with Fc gamma receptors and/or the complement system. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692.

In still other embodiments, the constant region is a glycosylated for N-linked glycosylation, e.g., the antibodies are engineered to bypass glycosylation. In some embodiments, the constant region is a glycosylated for N-linked glycosylation by mutating the oligosaccharide attachment residue and/or flanking residues that are part of the N-glycosylation recognition sequence in the constant region. For example, N-glycosylation site N297 may be mutated to, e.g., A, Q, K, or H. See, Tao et al., J. Immunology 143: 2595-2601, 1989; and Jefferis et al., Immunological Reviews 163:59-76, 1998. In some embodiments, the constant region is enzymatically aglycosylated for N-linked glycosylation (such as removing carbohydrate by the enzyme PNGase), or by expression in a glycosylation deficient host cell.

Other antibody modifications include antibodies that have been modified as described in PCT Publication No. WO 99/58572, which is hereby incorporated by reference in its entirety. These antibodies comprise, in addition to a binding domain directed at the target molecule, an effector domain having an amino acid sequence substantially homologous to all or part of a constant region of a human immunoglobulin heavy chain. These antibodies are capable of binding to the target molecule without triggering significant complement dependent lysis, or cell-mediated destruction of the target. In some embodiments, the effector domain is capable of specifically binding to FcRn and/or FcyRIIb. These are typically based on chimeric domains derived from two or more human immunoglobulin heavy chain CH2 domains. Antibodies modified in this manner are particularly suitable for use in chronic antibody therapy, to avoid inflammatory and other adverse reactions to conventional antibody therapy. In some embodiments, an antibody constant region can be modified to avoid interaction with Fc gamma receptors and/or the complement system. The techniques for preparation of such antibodies are described in WO 99/58572. For example, the constant region may be engineered to more resemble human constant regions to avoid immune response if the antibody is used in clinical trials and treatments in humans. See, e.g., U.S. Pat. Nos. 5,997,867 and 5,866,692, each of which are hereby incorporated by reference in their entirety.

The antibodies used in the IL-21 fusion proteins of the compositions and methods disclosed herein, listed by International Nonproprietary Name, can include, but are not limited to, abagovomab, abatacept, abciximababelacimab, abituzumab, abrezekimab, abrilumab, acapatamab, acasunlimab, acazicolcept, acrixolimab, actoxumab, adalimumab, adebrelimab, adecatumumab, adintrevimab, aducanumab, afasevikumab, afelimomab, aflibercept, alacizumab, alefacept, alemtuzumab, alirocumab, alnuctamab, alomfilimab, alsevalimab, altumomab, amatuximab, amivantamab, amlitelimab, amubarvimab, anatumomab, anbenitamab, andecaliximab, anetumab, anifrolumab, anivovetmab, anrukinzumab, anselamimab, ansuvimab, anumigilimab, apamistamab, apitegromab, apolizumab, aprutumab, arcitumomab, ascrinvacumab, aselizumab, asfotase, astegolimab, asunercept, atacicept, atezolizumab, atibuclimab, atidortoxumab, atinumab, atoltivimab, atorolimumab, avdoralimab, avelumab, avizakimab, axatilimab, azintuxizumab, bafisontamab, balstilimab, baminercept, bamlanivimab, bapineuzumab, bapotulimab, barecetamab, barzolvolimab, basiliximab, batiraxcept, batoclimab, bavituximab, bavunalimab, bebtelovimab, bectumomab, bedinvetmab, befovacimab, begelomab, belantamab, belatacept, belimumab, beludavimab, bemarituzumab, benralizumab, bentracimab, benufutamab, bepranemab, berlimatoxumab, bermekimab, bersanlimab, bertilimumab, besilesomab, betifisolimab, bevacizumab, bexmarilimab, bezlotoxumab, biciromab, bifikafusp, bimagrumab, bimekizumab, bintrafusp, birtamimab, bivatuzumab, bleselumab, blinatumomab, blisibimod, blontuvetmab, blosozumab, bococizumab, botensilimab, brazikumab, brentuximab, briakinumab, briobacept, briquilimab, brodalumab, brolucizumab, brontictuzumab, budigalimab, burfiralimab, burosumab, cabiralizumab, cadonilimab, camidanlumab, camoteskimab, camrelizumab, canakinumab, cantuzumab, caplacizumab, capromab, carlumab, carotuximab, casirivimab, catumaxomab, cedelizumab, cemiplimab, cendakimab, cergutuzumab, certolizumab, cetrelimab, cetuximab, cevostamab, cibisatamab, cifurtilimab, cilgavimab, cinpanemab, cinrebafusp, cirevetmab, citatuzumab, cixutumumab, clazakizumab, clenoliximab, clervonafusp, clesrovimab, clivatuzumab, cobolimab, codrituzumab, cofetuzumab, coltuximab, conatumumab, conbercept, concizumab, coprelotamab, cosfroviximab, cosibelimab, crefmirlimab, crenezumab, crexavibart, crizanlizumab, crotedumab, crovalimab, cudarolimab, cusatuzumab, dacetuzumab, daclizumab, dafsolimab, dalantercept, dalotuzumab, dalutrafusp, dapirolizumab, daratumumab, datopotamab, davoceticept, daxdilimab, dectrekumab, demcizumab, demupitamab, denintuzumab, denosumab, depatuxizumab, depemokimab, derlotuximab, detumomab, dezamizumab, dilpacimab, dinutuximab, diridavumab, disitamab, divozilimab, docaravimab, domagrozumab, domvanalimab, donanemab, dorlimomab, dostarlimab, dovanvetmab, dresbuxelimab, drozitumab, dulaglutide, duligotuzumab, dupilumab, durvalumab, dusigitumab, duvortuxizumab, ebdarokimab, eblasakimab, ebronucimab, ecleralimab, ecromeximab, eculizumab, edobacomab, edrecolomab, efalizumab, efanesoctocog, efaprinermin, efavaleukin, efbemalenograstim, efdamrofusp, efepoetin, efgartigimod, efgivanermin, efineptakin, efinopegdutide, efizonerimod, eflapegrastim, eflenograstim, eflepedocokin, eflimrufusp, efmarodocokin, efmitermant, efmoroctocog, efocipegtrutide, efpeglenatid, efpegsomatropin, efprezimod, efrilacedase, efruxifermin, eftansomatropin, eftilagimod, eftozanermin, eftrenonacog, efungumab, efzofitimod, eldelumab, elezanumab, elgemtumab, elipovimab, elotuzumab, elranatamab, elsilimomab, eluvixtamab, emactuzumab, emapalumab, emerfetamab, emfizatamab, emibetuzumab, emicizumab, emirodatamab, enapotamab, enavatuzumab, encelimab, enfortumab, enibarcimab, enlimomab, enoblituzumab, enokizumab, enoticumab, ensituximab, ensomafusp, enuzovimab, envafolimab, epcoritamab, epitumomab, epratuzumab, eptinezumab, eramkafusp, erenumab, erfonrilimab, erlizumab, ertumaxomab, etanercept, etaracizumab, etesevimab, etevritamab, etigilimab, etokimab, etrolizumab, evinacumab, evolocumab, evorpacept, exbivirumab, exidavnemab, ezabenlimab, fanolesomab, faralimomab, faricimab, farletuzumab, fasinumab, favezelimab, fazpilodemab, feladilimab, felvizumab, felzartamab, fezakinumab, fianlimab, ficlatuzumab, fidasimtamab, figitumumab, finotonlimab, firivumab, fiztasovimab, flanvotumab, fletikumab, flotetuzumab, fontolizumab, foralumab, foravirumab, fremanezumab, fresolimumab, frexalimab, frovocimab, frunevetmab, fulranumab, futuximab, galcanezumab, galegenimab, galiximab, gancotamab, ganitumab, gantenerumab, garadacimab, garetosmab, garivulimab, gatipotuzumab, gatralimab, gavilimomab, gedivumab, gefurulimab, gemtuzumab, geptanolimab, gevokizumab, giloralimab, gilvetmab, gimsilumab, ginisortamab, girentuximab, glembatumumab, glenzocimab, glofitamab, goflikicept, golimumab, golocdacimab, gontivimab, gosuranemab, gremubamab, gresonitamab, grisnilimab, gumokimab, guselkumab, ianalumab, ibalizumab, ibritumomab, icrucumab, idactamab, idarucizumab, ieramilimab, ifabotuzumab, ifinatamab, igovomab, iladatuzumab, imalumab, imaprelimab, imeiromab, imdevimab, imgatuzumab, imsidolimab, imvotamab, inbakicept, inclacumab, indatuximab, indusatumab, inebilizumab, inezetamab, infliximab, inolimomab, inotuzumab, intetumumab, ipafricept, iparomlimab, ipilimumab, iratumumab, isatuximab, iscalimab, isecarosmab, ispectamab, istiratumab, itepekimab, itolizumab, ivicentamab, ivonescimab, ivuxolimab, ixekizumab, izalontamab, izenivetmab, izuralimab, keliximab, labetuzumab, lacnotuzumab, lacutamab, ladiratuzumab, lampalizumab, lanadelumab, landogrozumab, laprituximab, larcaviximab, latikafusp, latozinemab, lebrikizumab, lecanemab, lemalesomab, lemzoparlimab, lenercept, lenvervimab, lenzilumab, lepunafusp, lerdelimumab, leronlimab, lesabelimab, lesofavumab, letaplimab, letolizumab, levilimab, lexatumumab, libivirumab, licaminlimab, lifastuzumab, ligelizumab, ligufalimab, lilotomab, lintuzumab, linvoseltamab, lirentelimab, lirilumab, litifilimab, livmoniplimab, lodapolimab, lodelcizumab, lokivetmab, lomtegovimab, loncastuximab, lonigutamab, lorigerlimab, lorukafusp, lorvotuzumab, losatuxizumab, lucatumumab, lulizumab, lumiliximab, lumretuzumab, lupartumab, luspatercept, lusvertikimab, lutikizumab, luveltamab, maftivimab, magrolimab, manelimab, manfidokimab, mapatumumab, margetuximab, marstacimab, masavibart, maslimomab, matuzumab, mavrilimumab, mazorelvimab, mecbotamab, melredableukin, melrilimab, mepolizumab, metelimumab, mezagitamab, mibavademab, milatuzumab, minretumomab, mipasetamab, miptenalimab, mirikizumab, miromavimab, mirvetuximab, mirzotamab, mitazalimab, mitumomab, modakafusp, modotuximab, mogamulizumab, monalizumab, morolimumab, mosunetuzumab, motavizumab, moxetumomab, mupadolimab, murlentamab, muromonab, nacolomab, nadecnemab, nadunolimab, namilumab, naptumomab, naratuximab, narnatumab, narsoplimab, natalizumab, navicixizumab, navivumab, naxitamab, nebacumab, necitumumab, nemolizumab, nepuvibart, nerelimomab, nesvacumab, netakimab, nimacimab, nimotuzumab, nipocalimab, nirsevimab, nivatrotamab, nivolumab, nofazinlimab, nofetumomab, nurulimab, obexelimab, obiltoxaximab, obinutuzumab, obrindatamab, ocaratuzumab, ociperlimab, ocrelizumab, odesivimab, odronextamab, odulimomab, ofatumumab, ogalvibart, olamkicept, olaratumab, oleclumab, olendalizumab, olinvacimab, olokizumab, omalizumab, omburtamab, omodenbamab, onartuzumab, onfekafusp, ongericimab, ontamalimab, ontorpacept, ontuxizumab, onvatilimab, opicinumab, opinercept, oportuzumab, opucolimab, ordesekimab, oregovomab, orilanolimab, ormutivimab, orticumab, osemitamab, osocimab, otelixizumab, otilimab, otlertuzumab, oxelumab, ozanezumab, ozoralizumab, ozuriftamab, pabinafusp, pacanalotamab, pacmilimab, pagibaximab, palivizumab, pamrevlumab, panitumumab, panobacumab, paridiprubart, parsatuzumab, pascolizumab, pasotuxizumab, pateclizumab, patritumab, pavurutamab, pelgifatamab, pembrolizumab, penpulimab, pepinemab, perakizumab, peresolimab, pertuzumab, petosemtamab, pexelizumab, pidilizumab, pimivalimab, pimurutamab, pinatuzumab, pintumomab, pivekimab, placulumab, plamotamab, plonmarlimab, plozalizumab, plutavimab, polatuzumab, ponezumab, ponsegromab, porgaviximab, posdinemab, pozelimab, praluzatamab, prasinezumab, prezalumab, priliximab, pritoxaximab, pritumumab, prolgolimab, pucotenlimab, pulocimab, quavonlimab, quetmolimab, quilizumab, quisovalimab, racotumomab, radretumab, rafivirumab, ragifilimab, ralpancizumab, ramatercept, ramucirumab, ranevetmab, ranibizumab, ravagalimab, ravulizumab, raxibacumab, recaticimab, refanezumab, regavirumab, regdanvimab, relatlimab, relfovetmab, remtolumab, reozalimab, reslizumab, retifanlimab, retlirafusp, revdofilimab, rilonacept, rilotumumab, rimteravimab, rinucumab, ripertamab, risankizumab, rituximab, rivabazumab, robatumumab, rocatinlimab, roledumab, rolinsatamab, romilkimab, romlusevimab, romosozumab, rontalizumab, rosmantuzumab, rosnilimab, rosopatamab, rovalpituzumab, rovelizumab, rozanolixizumab, rozibafusp, rulonilimab, runimotamab, ruplizumab, sabatolimab, sacituzumab, samalizumab, samrotamab, sarilumab, sasanlimab, satralizumab, satumomab, secukinumab, selicrelumab, semorinemab, semzuvolimab, serclutamab, seribantumab, serplulimab, setoxaximab, setrusumab, sevirumab, sibeprenlimab, sibrotuzumab, sifalimumab, siltuximab, simlukafusp, simridarlimab, simtuzumab, sintilimab, siplizumab, sirexatamab, sirtratumab, sirukumab, socazolimab, sofituzumab, solanezumab, solitomab, sonelokimab, sontuzumab, sotatercept, sotevtamab, sotigalimab, sotrovimab, sozinibercept, spartalizumab, spesolimab, stamulumab, suciraslimab, sudubrilimab, sugemalimab, sulesomab, suptavumab, surzebiclimab, sutimlimab, suvizumab, suvratoxumab, tabalumab, tabituximab, tacatuzumab, tadocizumab, tafasitamab, tafolecimab, tagitanlimab, talacotuzumab, talizumab, talquetamab, tamgiblimab, tamrintamab, tamtuvetmab, tanezumab, taplitumomab, tarcocimab, tarextumab, tarlatamab, tavolimab, tebentafusp, tebotelimab, tecaginlimab, teclistamab, tefibazumab, telazorlimab, telimomab, telisotuzumab, telitacicept, temelimab, tenatumomab, teneliximab, teplizumab, tepoditamab, teprotumumab, teropavimab, tesidolumab, tesnatilimab, tezepelumab, tibulizumab, tidutamab, tifcemalimab, tigatuzumab, tilavonemab, tildrakizumab, tilogotamab, tilvestamab, timigutuzumab, timolumab, tinurilimab, tiragolumab, tirnovetmab, tislelizumab, tisotumab, tixagevimab, tocilizumab, tomaralimab, tomuzotuximab, toralizumab, toripalimab, torudokimab, tosatoxumab, tositumomab, tovetumab, tozorakimab, tralokinumab, trastuzumab, trebananib, tregalizumab, tremelimumab, trinbelimab, trontinemab, tucotuzumab, tulinercept, tusamitamab, tuvirumab, tuvonralimab, ubamatamab, ublituximab, ulenistamab, uliledlimab, ulocuplumab, unasnemab, upanovimab, upifitamab, urabrelimab, urelumab, urtoxazumab, ustekinumab, utomilumab, vadastuximab, valanafusp, vandortuzumab, vantictumab, vanucizumab, vapaliximab, varisacumab, varlilumab, vatelizumab, vedolizumab, veltuzumab, vepalimomab, vepsitamab, vesencumab, vibecotamab, vibostolimab, vilobelimab, visilizumab, visugromab, vixarelimab, vixtimotamab, vobarilizumab, vobramitamab, vofatamab, volagidemab, volociximab, vonlerolizumab, vopratelimab, vorsetuzumab, votumumab, voxalatamab, vudalimab, vulinacimab, vunakizumab, xeligekimab, xentuzumab, zagotenemab, zalifrelimab, zalutumumab, zamerovimab, zampilimab, zanidatamab, zanolimumab, zansecimab, zelminemab, zeluvalimab, zenocutuzumab, zilovertamab, ziltivekimab, zimberelimab, zinlirvimab, ziralimumab, zolbetuximab, zolimomab, and zuberitamab. The antibodies, or fragments thereof, that can be used in the IL-21 fusion proteins of the compositions and methods disclosed herein are described in, for example, Ian Wilkinson & Geoff Hale (2022). Systematic analysis of the varied designs of 819 therapeutic antibodies and Fc fusion proteins assigned international nonproprietary names. mAbs. 14:1, 2123299. DOI: 10.1080/19420862.2022.2123299., which is hereby incorporated by reference in its entirety.

In some embodiments, the antibody or binding fragment thereof used in the IL-21 fusion proteins of the compositions and methods disclosed herein is an effectorless antibody or binding fragment thereof that has an isotype selected from the group consisting of NG, DANG, LALA, and/or LALA-PG mutations.

In some embodiments, the antibody or binding fragment thereof used in the IL-21 fusion protein of the compositions and methods disclosed herein, listed by International Nonproprietary Name, is an antibody selected from the group consisting of crefmirlimab, vibostolimab, and tifcemalumab, basiliximab, daclizumab, or a binding fragment thereof.

In some embodiments, the antibody or binding fragment thereof used in the IL-21 fusion proteins of the compositions and methods disclosed herein, is an antibody or binding fragment thereof that binds an antigen selected from the group consisting of human CD8A, human TIGIT, human CD25, human ICOS, human CD160, and human BTLA.

The antibody or binding fragment thereof used in the IL-21 fusion proteins of the compositions and methods disclosed herein can comprise, for example, an amino acid sequence 80%, 90%, 95%, 96%, 97%, 98%, 99%, or 100% identical to an amino acid sequence selected from the group consisting of SEQ ID NO: 5, 6, 7, 8, 9, 10, 11, 12, 13, or 14:

Crefmirlimab heavy chain variable domain
(SEQ ID NO: 5)
EVQLVESGGGLVQPGGSLRLSCAASGFNIKDTYIHFVRQAPGKGL
EWIGRIDPANDNTLYASKFQGKATISADTSKNTAYLQMNSLRAED
TAVYYCGRGYGYYVFDHWGQGTLVTVSS
Crefmirlimab light chain variable domain
(SEQ ID NO: 6)
DVQITQSPSSLSASVGDRVTITCRTSRSISQYLAWYQQKPGKVPK
LLIYSGSTLQSGVPSRFSGSGSGTDFTLTISSLQPEDVATYYCQQ
HNENPLTFGGGTKVEIK
Vibostolimab heavy chain variable domain
(SEQ ID NO: 7)
EVQLVQSGAEVKKPGSSVKVSCKASGYTFSSYVMHWVRQAPGQGL
EWIGYIDPYNDGAKYAQKFQGRVTLTSDKSTSTAYMELSSLRSED
TAVYYCARGGPYGWYFDVWGQGTTVTVSS
Vibostolimab light chain variable domain
(SEQ ID NO: 8)
DIQMTQSPSSLSASVGDRVTITCRASEHIYSYLSWYQQKPGKVPK
LLIYNAKTLAEGVPSRFSGSGSGTDFTLTISSLQPEDVATYYCQH
HFGSPLTFGQGTRLEIK
Tifcemalimab heavy chain variable domain
(SEQ ID NO: 9)
QVQLVQSGAEVKKPGASVKLSCKASGYNFKHTYAHWVRQAPGQGL
EWIGRIDPANGNTKYDPKFQGRATMTADTASNTAYLELSSLRSED
TAVYYCVADHYGSSLLDYWGQGTLVTVSS
Tifcemalimab light chain variable domain
(SEQ ID NO: 10)
DVVMTQTPLSLSVTPGQPASISCKSSQSLLDSDGKTYLNWFQQRP
GQSPRRLIYLVSKLDSGVPDRFSGSGSGTDFTLKISRVEAEDVGV
YYCWQGTYFPYTFGQGTKLEIK
Daclizumab heavy chain variable domain
(SEQ ID NO: 11)
QVQLVQSGAEVKKPGSSVKVSCKASGYTFTSYRMHWVRQAPGQGL
EWIGYINPSTGYTEYNQKFKDKATITADESTNTAYMELS SLRSE
DTAVYYCARGGGVEDYWGQGTLVTVSS
Daclizumab light chain variable domain
(SEQ ID NO: 12)
DIQMTQSPSTLSASVGDRVTITCSASSSISYMHWYQQKPGKAPKL
LIYTTSNLASGVPARFSGSGSGTEFTLTISSLQPDDFATYYCHQR
STYPLTFGQGTKVEVK
Basiliximab heavy chain variable domain
(SEQ ID NO: 13)
QLQQSGTVLARPGASVKMSCKASGYSFTRYWMHWIKQRPGQGLEW
IGAIYPGNSDTSYNQKFEGKAKLTAVTSASTAYMELSSLTHEDSA
VYYCSRDYGYYFDFWGQGTTLTVSS
Basiliximab light chain variable domain
(SEQ ID NO: 14)
QIVSTQSPAIMSASPGEKVTMTCSASSSRSYMQWYQQKPGTSPKR
WIYDTSKLASGVPARFSGSGSGTSYSLTISSMEAEDAATYYCHQR
SSYTFGGGTKLEIK

The IL-21 fusion proteins as described herein can be created by methods known in the art, for example, synthetic or recombinant methods. Typically, the fusion proteins are made by preparing and expressing a polynucleotide encoding them using recombinant methods, e.g., as described herein, although they may also be prepared by other methods known in the art, including, for example, chemical synthesis methods.

The antibodies as described herein can be made by any method known in the art. For the production of hybridoma cell lines, the route and schedule of immunization of the host animal are generally in keeping with established and conventional techniques for antibody stimulation and production, as further described herein. General techniques for producing human and mouse antibodies are known in the art and/or are described herein.

It is contemplated that any mammalian subject including humans, or antibody producing cells therefrom, can be manipulated to serve as the basis for production of mammalian, including human and hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C., Nature 256:495-497, 1975 or as modified by Buck, D. W., et al., In Vitro, 18:377-381, 1982. Available myeloma lines, including, but not limited to, X63-Ag8.653 and those from the Salk Institute, Cell Distribution Center, San Diego, Calif., USA, can be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBY immortalized B cells may be used to produce the monoclonal antibodies of the subject invention. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that can be used as a source of antibodies include all derivatives, and progeny cells, of the parent hybridomas that produce monoclonal antibodies.

Hybridomas that produce antibodies used for the present invention may be grown in vitro or in vivo using known procedures. The monoclonal antibodies may be isolated from the culture media or body fluids by conventional immunoglobulin purification procedures such as ammonium sulfate precipitation, gel electrophoresis, dialysis, chromatography, and ultrafiltration, if desired. Undesired activity, if present, can be removed, for example, by running the preparation over adsorbents made of the immunogen attached to a solid phase and eluting or releasing the desired antibodies off the immunogen. Immunization of a host animal with cells expressing the antibody target, a human target protein, or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOC12, or R1N═C═NR, where R and R1 are different alkyl groups, can yield a population of antibodies (e.g., monoclonal antibodies).

If desired, the antibody (monoclonal or polyclonal) of interest can be sequenced and the polynucleotide sequence may then be cloned into a vector for expression or propagation. The sequence encoding the antibody of interest can be maintained in a vector in a host cell and the host cell can then be expanded and frozen for future use. Production of recombinant monoclonal antibodies in cell culture can be carried out through cloning of antibody genes from B cells by means known in the art. See, e.g., Tiller et al., J. Immunol. Methods 329, 112, 2008; U.S. Pat. No. 7,314,622.

In some embodiments, antibodies as described herein are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416).

Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of ˜(1,4)-N-acetylglucosaminyltransferase III (GnTIII), a glycosyltransferase catalyzing formation of bisecting GicNAc, was reported to have improved ADCC activity (Umana et al., 1999, Nature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except praline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the tripeptide sequences (for N-linked glycosylation sites) described above. The alteration may also be made by the addition of, or substitution by, one or more serine or threonine residues to the sequence of the original antibody (for O-linked glycosylation sites).

The glycosylation pattern of antibodies can also be altered without altering the underlying nucleotide sequence. Glycosylation largely depends on the host cell used to express the antibody. Since the cell type used for expression of recombinant glycoproteins, e.g., antibodies, as potential therapeutics is rarely the native cell, variations in the glycosylation pattern of the antibodies can be expected (see, e.g., Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes, and the like.

Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example, using endoglycosidase H (Endo H), N-glycosidase F, endoglycosidase F 1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution, and chelation. Modifications can be used, for example, for attachment of labels for immunoassay. Modified polypeptides are made using established procedures in the art and can be screened using standard assays known in the art, some of which are described below and in the Examples.

The IL-21 fusion proteins and/or the IL-21 variants of this disclosure may be linked to a labeling agent such as a fluorescent molecule, a radioactive molecule, or any other labels known in the art. Labels are known in the art that provide (either directly or indirectly) a signal.

In some embodiments, the IL-21 fusion protein includes an antibody including or consisting of an Fc domain and an IL-21 variant having one or more specific substitutions at one or more, e.g., two or more, or three or more, or four or more, or five or more, or six or more, or seven or more, or eight or more, or nine or more, or ten or more of positions Q32, D33, R34, H35, M36, I37, R38, M39, Q41, L42, I43, D44, I45, V46, D47, Q48, L49, N51, Y52, V53, N54, L56, V57, E59, F60, L61, P62, A63, D66, E68, N70, E72, W73, S74, A75, F76, S77, F79, Q80, K85, S86, A87, N88, T89, E93, R94, I95, I96, N97, V98, S99, I100, L103, K104, R105, K106, P107, P108, S109, A112, R114, R115, H118, L120, K131, P132, P133, R139, S142, Q145, I148, Q150, H151, and L152 in the wild-type human IL-21 amino acid sequence (SEQ ID NO: 1).

In some embodiments, the IL-21 fusion protein includes an antibody including or consisting of a Fc domain and an IL-21 variant comprising one or more, e.g., two or more, or three or more, or four or more, or five or more, or six or more, or seven or more, or eight or more, or nine or more, or ten or more specific substitutions selected from the group consisting of: Q32K, D33A, D33N, D33P, D33Q, D33R, D33S, D33T, R34A, R34D, R34E, R34F, R34G, R34H, R34K, R34L, R34M, R34N, R34P, R34Q, R34S, R34T, R34V, R34W, R34Y, H35P, M36T, I37E, I37G, I37K, I37N, I37Q, I37R, I37S, I37V, R38A, R38E, R38F, R38K, R38M, R38Q, R38V, M39D, M39E, M39G, M39I, M39K, M39P, M39Q, M39R, Q41K, Q41P, Q41S, L42D, L42E, L42G, L42H, L42K, L42N, L42Q, L42R, L42S, L42Y, I43D, I43L, I43M, I43P, I43S, D44A, D44G, D44N, D44P, D44T, I45A, I45D, I45G, I45H, I45N, I45P, I45R, I45S, I45Y, V46D, V46E, V46G, V46K, V46N, V46P, V46Q, V46T, D47P, D47S, Q48G, Q48K, L49D, L49E, L49H, L49K, L49N, L49P, L49R, L49S, N51E, N51G, N51P, N51V, Y52D, Y52H, Y52K, Y52N, Y52Q, V53A, V53E, V53H, V53N, V53P, V53Q, V53S, V53T, N54H, N54K, N54P, L56E, L56F, L56M, L56N, L56S, L56T, V57H, V57L, V57M, V57P, V57R, V57T, V57Y, E59R, E59S, F60H, F60I, F60R, L61H, L61I, P62H, P62S, P62T, P62V, A63T, D66M, D66R, E68K, E68R, N70A, N70H, E72H, E72I, E72L, E72S, E72T, W73A, W73G, W73K, W73L, W73N, W73P, W73Q, W73S, W73T, W73V, W73Y, S74A, S74H, S74N, A75N, F76D, F76E, F76H, F76M, F76P, F76Q, F76R, F76T, S77K, S77M, S77R, F79D, F79E, F79K, F79P, F79R, F79T, F79Y, Q80A, Q80D, K85S, S86I, A87N, N88K, T89L, E93G, E93T, R94S, R94T, I95H, I95K, I95P, I95T, I96A, I96D, I96G, I96P, I96S, I96T, I96V, N97G, V98H, V98K, V98R, S99H, I100E, L103E, K104G, R105A, R105D, R105K, R105Q, R105S, R105W, K106E, K106N, K106P, K106S, K106T, P107D, P107Y, P108D, P108E, P108G, P108I, P108L, P108Q, P108T, S109K, S109M, S109R, A112P, A112R, R114F, R115I, R115P, R115Q, H118I, H118K, H118R, L120H, L120K, L120M, L120V, L120Y, K131M, K131V, P132A, P133I, R139S, S142D, S142H, S142I, Q145H, I148S, Q150D, Q150R, Q150S, H151E, and L152H in the wild-type human IL-21 amino acid sequence (SEQ ID NO: 1).

In some embodiments, the IL-21 fusion protein includes an antibody including or consisting of an Fc domain and an IL-21 variant having one or more specific substitutions at one or more, e.g., two or more, three or more, four or more, five or more, six or more, seven or more, eight or more, or nine or more, of positions H35, N91, K101, N111, R114, R115, H118, R119, T121 in the wild-type human IL-21 amino acid sequence (SEQ ID NO: 1).

In some embodiments, the IL-21 fusion protein includes an antibody including or consisting of a Fc domain and an IL-21 variant comprising one or more, e.g., two or more, or three or more, or four or more, or five or more, or six or more, or seven or more, or eight or more, or nine or more specific substitutions selected from the group consisting of H35F, N91E, K101E, N111E, R114G, R115G, H118D, R119T, and T121E in the wild-type human IL-21 amino acid sequence (SEQ ID NO: 1).

In some embodiments, the IL-21 fusion protein includes an antibody including or consisting of a Fc domain and an IL-21 variant comprising the amino acid substitutions H35F, N91E, K101E, N111E, R114G, R115G, H118D, R119T, and T121E in the wild-type human IL-21 amino acid sequence (SEQ ID NO: 1). In some embodiments, the IL-21 fusion protein includes an antibody including or consisting of a Fc domain and an IL-21 variant comprising the amino acid substitutions H35F, N91E, and K101E in the wild-type human IL-21 amino acid sequence (SEQ ID NO: 1). In some embodiments, the IL-21 fusion protein includes an antibody including or consisting of a Fc domain and an IL-21 variant comprising the amino acid substitutions N111E, R114G, R115G, H118D, R119T, and T121E in the wild-type human IL-21 amino acid sequence (SEQ ID NO: 1). In some embodiments, the IL-21 fusion protein includes an antibody including or consisting of a Fc domain and an IL-21 variant comprising the amino acid substitutions N111E, R115G, H118D, and T121E in the wild-type human IL-21 amino acid sequence (SEQ ID NO: 1).

In some embodiments, the IL-21 fusion protein includes an antibody including or consisting of a Fc domain and an IL-21 variant comprising the amino acid substitutions H35F, R114G, R115G, H118D, R119T, and T121E in the wild-type human IL-21 amino acid sequence (SEQ ID NO: 1).

In some embodiments, the IL-21 fusion protein includes an antibody including or consisting of an Fc domain and an IL-21 variant having one or more specific substitutions at one or more, e.g., two or more, three or more, four or more, five or more, six or more, seven or more, eight or more, nine or more, ten or more, 11 or more, 12 or more, or 13 of positions H35, I45, N70, K81, V98, P107, T110, A112, R114, R115, H118, R119, T121 in the wild-type human IL-21 amino acid sequence (SEQ ID NO: 1).

In some embodiments, the IL-21 fusion protein includes an antibody including or consisting of an Fc domain and an IL-21 variant having one or more specific substitutions at one or more, e.g., two or more, three or more, four or more, five or more, six or more, seven or more, eight or more, nine or more, ten or more, 11 or more, 12 or more, 13 or more, 14 or more, 15 or more, or 16 specific substitutions selected from the group consisting of H35F, H35I, I45T, N70G, K81D, V98E, P107V, T110D, A112P, R114Q, R114G, R115D, R115G, H118D, R119T, and T121E in the wild-type human IL-21 amino acid sequence (SEQ ID NO: 1).

In some embodiments, the IL-21 fusion protein has a binding affinity to human IL-21R that is decreased by 1.5-fold, 2-fold, 3-fold, 5-fold, 10-fold, 50-fold, 100-fold, 500-fold, 1000-fold, 2000-fold, or 3000-fold, or more relative to the binding affinity between the wild-type human IL-21 polypeptide and the human IL-21R.

### Polynucleotides, Vectors, Host Cells

The disclosure also provides polynucleotides encoding any of the IL-21 variants and IL-21 fusion proteins as described herein. In one aspect, the disclosure provides methods of making any of the polynucleotides described herein. Polynucleotides can be made and expressed by procedures known in the art.

In another aspect, the disclosure provides compositions (such as pharmaceutical compositions) including any of the polynucleotides described herein. In some embodiments, the composition includes an expression vector having a polynucleotide encoding any of the IL-21 variants and IL-21 fusion proteins described herein.

In another aspect, the disclosure provides isolated cell lines that produce the IL-21 variants and the IL-21 fusion proteins as described herein. In some embodiments, the cell line is an engineered immune cell, wherein the engineered immune cell includes a chimeric antigen receptor (CAR). In some embodiments, the IL-21 variants and the IL-21 fusion proteins, when expressed as polynucleotides in CAR T cells, either as secreted or membrane-tethered versions, are used to enhance CAR T function, including activity and proliferation.

Immune cells producing the IL-21 variants and the IL-21 fusion proteins as described herein can be made by introducing a CAR into immune cells, and expanding the cells. For example, the immune cells can be engineered by providing a cell and expressing at the surface of the cell at least one CAR and at least one IL-21 variant or IL-21 fusion protein as described herein. Methods for engineering immune cells are described in, for example, PCT Patent Application Publication Nos. WO/2014/039523, WO/2014/184741, WO/2014/191128, WO/2014/184744, and WO/2014/184143, each of which is incorporated herein by reference in its entirety. In some embodiments, the cell can be transformed with at least one polynucleotide encoding a CAR, one polynucleotide encoding the IL-21 variant or IL-21 fusion protein as described herein, followed by expressing the polynucleotides in the cell.

Polynucleotides complementary to any such sequences are also encompassed by the present invention. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide described herein, and a polynucleotide may, but need not, be linked to other molecules and/or support materials. Polynucleotides can include a native sequence (e.g., an endogenous sequence that encodes an antibody or a fragment thereof) or may include a variant of such a sequence. Polynucleotide variants of antibodies or fragments thereof contain one or more substitutions, additions, deletions, and/or insertions such that the immunoreactivity of the encoded polypeptide is not diminished, relative to a native immunoreactive molecule. The effect on the immunoreactivity of the encoded polypeptide may generally be assessed as described herein and by methods known in the relevant field. Variants of antibodies or fragments thereof can exhibit at least about 70% identity, at least about 80% identity, at least about 90% identity, or at least about 95, 96, 97, 98, or 99% identity to a polynucleotide sequence that encodes a native antibody or a fragment thereof.

Variants are substantially homologous to a native gene, or a portion or complement thereof. It will be appreciated by those of ordinary skill in the art that, because of the degeneracy of the genetic code, there are many nucleotide sequences that encode polypeptides as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered because of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence. For preparing polynucleotides using recombinant methods, a polynucleotide having a desired sequence can be inserted into a suitable vector, and the vector in turn can be introduced into a suitable host cell for replication and amplification, as further discussed herein. Polynucleotides can be inserted into host cells by any means known in the art. Cells are transformed by introducing an exogenous polynucleotide by direct uptake, endocytosis, transfection, F-mating, or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

As is well known, PCR allows reproduction of DNA sequences. See, e.g. U.S. Pat. Nos. 4,683,195, 4,800,159, 4,754,065 and 4,683,202, as well as PCR: The Polymerase Chain Reaction, Mullis et al. eds., Birkauswer Press, Boston, 1994. RNA can be obtained by using isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors can be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp 18, mp 19, pBR322, pMB9, ColE1, pCRl, RP4, phage DNAs, and shuttle vectors such as pSA3 and pAT28. These and many other cloning vectors are available from commercial vendors such as BioRad, Strategene, and Invitrogen.

Expression vectors are also provided herein. Expression vectors generally are replicable polynucleotide constructs that contain a polynucleotide as described herein. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate methods, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of how to introduce vectors or polynucleotides into cells will often depend on features of the host cell.

The disclosure also provides host cells including any of the polynucleotides described herein. Any host cells capable of over-expressing heterologous DNAs can be used for the purpose of isolating the genes encoding the antibody, polypeptide, or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtilis) and yeast (such as S. cerevisae, S. pombe; or K. lactis). Preferably, the host cells express the cDNAs at a level of about 5 fold higher, 10 fold higher, or 20 fold higher than that of the corresponding endogenous antibody or protein of interest, if present, in the host cells. Screening the host cells for a specific binding to IL-21 or a IL-21 domain can be effected by an immunoassay or FACS. A cell overexpressing the antibody or protein of interest can be identified.

An expression vector can be used to direct expression of an IL-21 variant or an IL-21 fusion protein. One skilled in the art is familiar with administration of expression vectors to obtain expression of an exogenous protein in vivo. See, e.g., U.S. Pat. Nos. 6,436,908; 6,413,942; and 6,376,471. Administration of expression vectors includes local or systemic administration, including injection, oral administration, particle gun, catheterized administration, and topical administration. In another embodiment, the expression vector is administered directly to the sympathetic trunk or ganglion, or into a coronary artery, atrium, ventricle, or pericardium.

Targeted delivery of therapeutic compositions containing an expression vector, or subgenomic polynucleotides can also be used. Receptor-mediated DNA delivery techniques are described in, for example, Findeis et al., Trends Biotechnol., 1993, 11:202; Chiou et al., Gene Therapeutics: Methods And Applications Of Direct Gene Transfer, J. A. Wolff, ed., 1994; Wu et al., J. Biol. Chem., 1988, 263:621; Wu et al., J. Biol. Chem., 1994, 269:542; Zenke et al., Proc. Natl. Acad. Sci. USA, 1990, 87:3655; Wu et al., J. Biol. Chem., 1991, 266:338.

Therapeutic compositions containing a polynucleotide are administered in a range of about 100 ng to about 200 mg of DNA for local administration in a gene therapy protocol. Concentration ranges of about 500 ng to about 50 mg, about 1 pg to about 2 mg, about 5 pg to about 500 pg, and about 20 pg to about 100 pg of DNA can also be used during a gene therapy protocol. The therapeutic polynucleotides and polypeptides can be delivered using gene delivery vehicles. The gene delivery vehicle can be of viral or non-viral origin (see generally, Jolly, Cancer Gene Therapy, 1994, 1:51; Kimura, Human Gene Therapy, 1994, 5:845; Connelly, Human Gene Therapy, 1995, 1:185; and Kaplitt, Nature Genetics, 1994, 6:148).

Expression of such coding sequences can be induced using endogenous mammalian or heterologous promoters. Expression of the coding sequence can be either constitutive or regulated. Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936: WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3: 147 can also be employed.

Non-viral delivery vehicles and methods can also be employed, including, but not limited to, polycationic condensed DNA linked or unlinked to killed adenovirus alone (see, e.g., Curiel, Hum. Gene Ther., 1992, 3:147); ligand-linked DNA (see, e.g., Wu, J. Biol. Chem., 1989, 264: 16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 1994, 14:2411, and in Woffendin, Proc. Natl. Acad. Sci., 1994, 91:1581.

### Compositions

The disclosure also provides pharmaceutical compositions including an effective amount of an IL-21 variant or an IL-21 fusion protein as described herein. Examples of such compositions, as well as how to formulate, are also described herein. In some embodiments, the composition includes one or more IL-21 variants, combinations with one or more other variant detuned cytokines and/or other IL-21 fusion proteins.

In some embodiments, the compositions include an IL-21 fusion protein including an antibody and a human IL-21 variant having one or more, e.g., two or more, or three or more, or four or more, or five or more, or six or more, or seven or more, or eight or more, or nine or more, or ten or more specific substitutions in SEQ ID NO: 1 as described herein.

In certain embodiments, the compositions can include more than one IL-21 variant or IL-21 fusion protein (e.g., a mixture of IL-21 variants or IL-21 fusion proteins comprising different IL-21 variants and/or different antibodies).

The compositions disclosed herein can further include pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at known dosages and concentrations, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG). Pharmaceutically acceptable excipients are further described herein.

The IL-21 variants, IL-21 fusion proteins, and compositions thereof can also be used in conjunction with, or administered separately, simultaneously, or sequentially with other agents that serve to enhance and/or complement the effectiveness of the agents. The disclosure also provides compositions, including pharmaceutical compositions, including any of the polynucleotides described herein. In some embodiments, the compositions include an expression vector including a polynucleotide encoding the IL-21 variants and IL-21 fusion proteins as described herein. In other embodiments, the compositions include an expression vector having a polynucleotide encoding any of the IL-21 variants and IL-21 fusion proteins described herein.

### Methods of Treatment

The IL-21 variants and the IL-21 fusion proteins described herein are useful in various applications including, but are not limited to, therapeutic treatment methods and diagnostic treatment methods.

In one aspect, the disclosure provides methods for treating cancer. In some embodiments, the methods of treating cancer in a subject include administering to the subject in need thereof a therapeutically effective amount, e.g., an amount effective to provide a reduction in tumor size or volume over time, and/or an amount effective to reduce one or more symptoms caused by the cancer, of a composition (e.g., a pharmaceutical composition) including any of the IL-21 variants and the IL-21 fusion proteins as described herein. As used herein, a cancer can be a solid cancer or a blood or bone marrow cancer. Solid cancers include, but are not limited to, gastric cancer, small intestine cancer, sarcoma, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer, mesothelioma, ovarian cancer, breast cancer, prostate cancer, esophageal cancer, pancreatic cancer, glioma, renal cancer (e.g., renal cell carcinoma), bladder cancer, cervical cancer, uterine cancer, vulvar cancer, penile cancer, testicular cancer, anal cancer, choriocarcinoma, colorectal cancer, oral cancer, skin cancer, Merkel cell carcinoma, glioblastoma, brain tumor, bone cancer, eye cancer, and melanoma.

Blood and bone marrow cancers include, but are not limited to, multiple myeloma, malignant plasma cell neoplasm, Hodgkin's lymphoma, nodular lymphocyte predominant Hodgkin's lymphoma, Kahler's disease and Myelomatosis, plasma cell leukemia, plasmacytoma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, follicular lymphoma, marginal zone lymphoma, mucosa-associated lymphatic tissue lymphoma, small cell lymphocytic lymphoma, mantle cell lymphoma, Burkitt lymphoma, primary mediastinal (thymic) large B-cell lymphoma, lymphoplasmactyic lymphoma, Waldenstrom macroglobulinemia, nodal marginal zone B cell lymphoma, splenic marginal zone lymphoma, intravascular large B-cell lymphoma, primary effusion lymphoma, lymphomatoid granulomatosis, T cell histiocyte-rich large B-cell lymphoma, primary central nervous system lymphoma, primary cutaneous diffuse large B-cell lymphoma (leg type), EBY positive diffuse large B-cell lymphoma of the elderly, diffuse large B-cell lymphoma associated with inflammation, intravascular large B-cell lymphoma, ALK-positive large B-cell lymphoma, plasmablastic lymphoma, large B-cell lymphoma arising in HHVS-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other hematopoietic cell related cancer. In some embodiments, the cancer is relapsed, refractory, or metastatic.

In some embodiments, the methods of inhibiting tumor growth or progression in a subject include administering to the subject in need thereof an effective amount, e.g., a therapeutically effective amount, of a composition including the IL-21 variants or IL-21 fusion proteins as described herein. In some embodiments, the disclosure includes methods of inhibiting metastasis of cancer cells in a subject, which include administering to a subject in need thereof an effective amount of a composition including any of the IL-21 variants or IL-21 fusion proteins as described herein. In other embodiments, the disclosure includes methods of inducing regression of a tumor in a subject, which include administering to the subject in need thereof an effective amount of a composition including any of the IL-21 variants or IL-21 fusion proteins as described herein. In another aspect, the disclosure provides methods of detecting, diagnosing, and/or monitoring a cancer. For example, the IL-21 variants or IL-21 fusion proteins as described herein can be labeled with a detectable moiety such as an imaging agent and an enzyme-substrate label. The IL-21 variants or IL-21 fusion proteins as described herein can also be used for in vivo diagnostic assays, such as in vivo imaging (e.g., PET or SPECT), or a staining reagent.

In some embodiments, the methods described herein further include a step of treating a subject with an additional form of therapy. In some embodiments, the additional form of therapy is an additional anti-cancer therapy including, but not limited to, chemotherapy, radiation, surgery, hormone therapy, checkpoint inhibitor therapy, and/or additional immunotherapy.

With respect to all methods described herein, reference to IL-21 variants or IL-21 fusion proteins also includes compositions comprising one or more additional agents. These compositions may further comprise suitable excipients, such as pharmaceutically acceptable excipients including buffers, which are well known in the art. The methods and compositions described herein can be used alone or in combination with other methods of treatment.

The IL-21 variants and/or IL-21 fusion proteins as described herein can be administered to a subject via any suitable route. It should be apparent to a person skilled in the art that the examples described herein are not intended to be limiting, but to be illustrative of the techniques available. Accordingly, in some embodiments, the IL-21 variant or IL-21 fusion protein is administered to a subject in accord with known methods, such as intravenous administration, e.g., as a bolus or by continuous infusion over a period of time, by intramuscular, intraperitoneal, intracerebrospinal, transdermal, subcutaneous, intra-articular, sublingually, or intrasynovial administration, or via insufflation, intrathecal, oral, inhalation, or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized, and lyophilized powder can be nebulized after reconstitution. Alternatively, the IL-21 variants or IL-21 fusion proteins can be aerosolized using a fluorocarbon formulation and a metered dose inhaler, or inhaled as a lyophilized and milled powder.

In some embodiments, an IL-21 variant or IL-21 fusion protein is administered via site-specific or targeted local delivery techniques. Examples of site-specific or targeted local delivery techniques include direct injection into patient tumor or various implantable depot sources of the IL-21 variants or IL-21 fusion proteins or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Various formulations of an IL-21 variant or IL-21 fusion protein can be used for administration. In some embodiments, the IL-21 variant or IL-21 fusion protein can be administered neat. In some embodiments, the IL-21 variant or IL-21 fusion protein and a pharmaceutically acceptable excipient may be in various formulations. Pharmaceutically acceptable excipients are known in the art and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing, 2000. In some embodiments, these agents are formulated for administration by injection (e.g., intraperitoneally, intravenously, subcutaneously, intramuscularly, etc.). Accordingly, these agents can be combined with pharmaceutically acceptable vehicles such as saline, Ringer's solution, dextrose solution, and the like. The particular dosage regimen, i.e., dose, timing and repetition, will depend on the particular individual and that individual's medical history.

Generally, for administration of IL-21 variants or IL-21 fusion proteins, the candidate dosage can be administered daily, every other day, every third day, every week, every other week, every three weeks, every four weeks, every five weeks, every six weeks, every seven weeks, every eight weeks, every ten weeks, every twelve weeks, or more than every twelve weeks. For repeated administrations over several days or longer, depending on the condition, the treatment is sustained until a desired suppression of symptoms occurs or until sufficient therapeutic levels are achieved, for example, to reduce symptoms associated with cancer. The progress of this therapy is easily monitored by conventional techniques and assays. The dosing regimen (including the specific IL-21 variants or IL-21 fusion proteins used) can vary over time.

In some embodiments, the candidate dosage is administered daily, weekly, every two weeks, every three weeks, every four weeks, or monthly, with the dosage ranging from about 0.01 mg/kg, about 0.1 mg/kg, about 1 mg/kg, about 10 mg/kg, about 50 mg/kg, to about 100 mg/kg, or dosages therebetween.

In some embodiments, the candidate dosage is administered daily, weekly, every two weeks, every three weeks, every four weeks, or monthly, with the administered unit dosage ranging from about 0.01 mg, about 0.1 mg, about 1 mg, about 10 mg, about 50 mg, about 100 mg, to about 1000 mg, or dosages therebetween.

For the purposes of the present disclosure, the appropriate dosage of an IL-21 variant or an IL-21 fusion protein will depend on the IL-21 variant or an IL-21 fusion protein (or compositions thereof) employed, the type and severity of symptoms to be treated, whether the agent is administered for preventive or therapeutic purposes, previous therapy, the patient's clinical history and response to the agent, the patient's clearance rate for the administered agent, and the discretion of the attending physician. Typically, the clinician will administer an IL-21 variant or an IL-21 fusion protein until a dosage is reached that achieves the desired result. Dose and/or frequency can vary over the course of treatment. In some embodiments, step dosing is performed where the initial dose or doses are lower than incrementally higher doses administered as the regimen continues. Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of symptoms. Alternatively, sustained continuous release formulations of IL-21 variants or IL-21 fusion proteins may be appropriate. Various formulations and devices for achieving sustained release are known in the art.

In one embodiment, dosages for an IL-21 variant or an IL-21 fusion protein may be determined empirically in individuals who have been given one or more administration (s) of an IL-21 variant or an IL-21 fusion protein. For example, individuals are given incremental dosages of an IL-21 variant or an IL-21 fusion protein. To assess efficacy, an indicator of the disease can be followed. Administration of an IL-21 variant or an IL-21 fusion protein as described herein in accordance with the method in the present invention can be continuous or intermittent, depending, for example, upon the recipient's physiological condition, whether the purpose of the administration is therapeutic or prophylactic, and other factors known to skilled practitioners. The administration of an IL-21 variant or an IL-21 fusion protein may be essentially continuous over a preselected period of time or may be in a series of spaced doses. In some embodiments, more than one IL-21 variant or IL-21 fusion protein may be present. At least one, at least two, at least three, at least four, at least five, or more different IL-21 variants or IL-21 fusion proteins can be present. Generally, those IL-21 variants or IL-21 fusion proteins may have complementary activities that do not adversely affect each other.

In some embodiments, the IL-21 variant or the IL-21 fusion protein may be administered in combination with one or more additional therapeutic agents. These include, but are not limited to, a biotherapeutic agent, a chemotherapeutic agent, a vaccine, a CAR-T cell-based therapy, radiotherapy, another cytokine therapy (e.g., immunostimulatory cytokines including various signaling proteins that stimulate immune response, such as interferons, interleukins, and hematopoietic growth factors), a vaccine, an inhibitor of other immunosuppressive pathways, e.g., anti-PD-1 checkpoint inhibitors, an inhibitors of angiogenesis, a T cell activator, an inhibitor of a metabolic pathway, an mTOR (mechanistic target of rapamycin) inhibitor (e.g., rapamycin, rapamycin derivatives, sirolimus, temsirolimus, everolimus, and deforolimus), an inhibitor of an adenosine pathway, a tyrosine kinase inhibitor including but not limited to inlyta, ALK (anaplastic lymphoma kinase) inhibitors (e.g., crizotinib, ceritinib, alectinib, and sunitinib), a BRAF inhibitor (e.g., vemurafenib and dabrafenib), an epigenetic modifier, an inhibitors or depletor of Treg cells and/or of myeloid-derived suppressor cells, a JAK (Janus Kinase) inhibitor (e.g., ruxolitinib and tofacitinb, varicitinib, filgotinib, gandotinib, lestaurtinib, momelotinib, pacritinib, and upadacitinib), a cyclin-dependent kinase inhibitor, an immunogenic agent (for example, attenuated cancerous cells, tumor antigens, antigen presenting cells such as dendritic cells pulsed with tumor derived antigen or nucleic acids, a MEK inhibitor (e.g., trametinib, cobimetinib, binimetinib, and selumetinib), a GLSl inhibitor, a PAP inhibitor, an oncolytic virus, an IDO (Indoleamine-pyrrole 2,3-dioxygenase) inhibitor, a PRR (Pattern Recognition Receptors) agonist, and cells transfected with genes encoding immune stimulating cytokines such as but not limited to GM-CSF).

In some embodiments, examples of immunostimulatory cytokines include, but are not limited to, GM-CSF, G-CSF, IFNy, IFNa; IL-2 (e.g. denileukin difitox), IL-4, IL-6, IL-7, IL-10, IL-11, IL-12, IL-15, IFNA2, IL-18, and TNFa. In some embodiments, the cytokines are pegylated (e.g., pegylated IL-2, IL-10, IFNy, and IFNa).

Pattern recognition receptors (PRRs) are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-I-like receptors (RLRs), nucleotide-binding oligomerization domain (NOD)-like receptors (NLRs), C-type lectin receptors (CLRs), and Stimulator of Interferon Genes (STING) protein. The terms “TLR” and “toll-like receptor” refer to any toll-like receptor. Toll-like receptors are receptors involved in activating immune responses. TLRs recognize, for example, pathogen-associated molecular patterns (PAMPs) expressed in microbes, as well as endogenous damage-associated molecular patterns (DAMPs), which are released from dead or dying cells.

Molecules that activate TLRs (and thereby activate immune responses) are referred to herein as “TLR agonists.” TLR agonists can include, for example, small molecules (e.g. organic molecule having a molecular weight under about 1000 Daltons), as well as large molecules (e.g. oligonucleotides and proteins). Some TLR agonists are specific for a single type of TLR (e.g. TLR3 or TLR9), while some TLR agonists activate two or more types of TLR (e.g. both TLR7 and TLRS).

Examples of TLR agonists provided herein include agonists of TLR2, TLR3, TLR4, TLR5, TLR6, TLR7, TLRS, and TLR9.

Examples of small molecule TLR agonists include those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; 6,818,650; and 7,7091,214; U.S. Patent Publication Nos. 2004/0091491, 2004/0176367, and 2006/0100229; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, WO 2005/079195, WO 2005/094531, WO 2005/123079, WO 2005/123080, WO 2006/009826, WO 2006/009832, WO 2006/026760, WO 2006/028451, WO 2006/028545, WO 2006/028962, WO 2006/029115, WO 2006/038923, WO 2006/065280, WO 2006/074003, WO 2006/083440, WO 2006/086449, WO 2006/091394, WO 2006/086633, WO 2006/086634, WO 2006/091567, WO 2006/091568, WO 2006/091647, WO 2006/093514, and WO 2006/098852.

Additional examples of small molecule TLR agonists include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-.beta.-D-ribofuranosylthiazolo [4,5-d]pyrimidine derivatives (such as those described in U.S. Publication No. 2003/0199461), and certain small molecule immuno-potentiator compounds such as those described, for example, in U.S. Patent Publication No. 2005/0136065.

Examples of large molecule TLR agonists include TLR agonist oligonucleotide sequences. Some TLR agonist oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other TLR agonist nucleotide sequences lack CpG sequences and are described, for example, in International Patent Publication No. WO 00/75304. Still other TLR agonist nucleotide sequences include guanosine- and uridine-rich single-stranded RNA (ssRNA) such as those described, for example, in Heil et ah, Science, vol. 303, pp. 1526-1529, Mar. 5, 2004. Other TLR agonists include biological molecules such as aminoalkyl glucosaminide phosphates (AGPs) and are described, for example, in U.S. Pat. Nos. 6,113,918; 6,303,347; 6,525,028; and 6,649,172.

TLR agonists also include inactivated pathogens or fractions thereof, which may activate multiple different types of TLR receptor. Examples of pathogen-derived TLR agonists include BCG, Mycobacterium obuense extract, Talimogene laherparepvec (T-Vec) (derived from HSV-1), and Pexa-Vec (derived from vaccina virus).

In some embodiments, a TLR agonist may be an agonist antibody that binds specifically to the TLR.

In some embodiments, the biotherapeutic agent is an antibody, including but not limited to, an anti-CTLA-4 antibody, an anti-CD3 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD40 antibody, an anti-CD40L antibody, anti-CD47 antibody, an anti-CSFlR antibody, an anti-CSFl antibody, an anti-IL-7R antibody, an anti-MARCO antibody, an anti-CXCR4 antibodies, an anti-VEGF antibody, an anti-VEGFRl antibody, an anti-VEGFR2 antibody, an anti-TNFRl antibody, an antiTNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD52 antibody, an anti-CCR4 antibody, an anti-CCRS antibody, an anti-CD200R antibody, an antiVISG4 antibody, an anti-CCR2 antibody, an anti-LILRb2 antibody, an anti-CXCR4 antibody, an anti-CD206 antibody, an anti-CD163 antibody, an anti-KLRGl antibody, an antiFLT3 antibody, an anti-B7-H4 antibody, an anti-B7-H3 antibody, an KLRG1 antibody, a BTNlAl antibody, a BCMA antibody, a CLEC9A antibody, a LILRB4 antibody, or an anti-GITR antibody.

In some embodiments, an IL-21 variant or an IL-21 fusion protein is used in combination with an immunocytokine. In some embodiments, the immunocytokine includes an antibody, or fragment thereof, conjugated or fused to a cytokine (e.g., fusion protein). In some embodiments, the antibody, or fragment thereof, binds to the Extra Domain-A (EDA) isoform of fibronectin (e.g., anti-EDA antibody). Accordingly, in some embodiments, an IL-21 variant or an IL-21 fusion protein is used in conjunction with, for example, an anti-PD-LI antagonist antibody; an anti-PD-I antagonist antibody such as for example, nivolumab (OPDIVO®), pembrolizumab (KEYTRUDA®), mAb7 (e.g., as described in US Pub. No. US20160159905, hereby incorporated by reference), and pidilizumab; an anti-CTLA-4 antagonist antibody such as for example ipilimumab (YERVOY®); an anti-LAG-3 antagonist antibody such as BMS-9860i6 and IMP70i; an anti-TIM-3 antagonist antibody; an anti-B7-H3 antagonist antibody such as for example MGA271; an-anti-VISTA antagonist antibody; an anti-TIGIT antagonist antibody; an anti-CD28 antagonist antibody; an anti-CD8 antibody; an anti-CD86 antibody; an anti-B7-H4 antagonist antibody; an anti-ICOS agonist antibody; an anti-CD28 agonist antibody; an innate immune response modulator (e.g., TLRs, KIR, NKG2A), and an IDO inhibitor.

In some embodiments, an IL-21 variant or an IL-21 fusion protein is used in conjunction with a 4-18B (CD137) agonist such as, for example, PF-05082566 or urelumab (BMS-663513). In some embodiments, an IL-21 variant or an IL-21 fusion protein is used in conjunction with an OX40 agonist such as, for example, an anti-OX-40 agonist antibody. In some embodiments, an IL-21 variant or an IL-21 fusion protein is used in conjunction with a GITR agonist such as, for example, TRX518. In some embodiments, an IL-21 variant or an IL-21 fusion protein is used in conjunction with an IDO inhibitor. In some embodiments, an IL-21 variant or an IL-21 fusion protein is used in conjunction with a cytokine therapy such as, for example without limitation, (pegylated or non-pegylated) IL-2, IL-10, IL-12, IL-7, IL-15, IL-21, IL-33, CSF-1, MCSF-1, etc.

In some embodiments, other examples of the antibody for the combined use with the IL-21 variant or the IL-21 fusion protein of the present invention can be directed to, 5T4; A33; alpha-folate receptor 1 (e.g., mirvetuximab soravtansine); Alk-1; CA-125 (e.g., abagovomab); Carboanhydrase IX; CCR2; CCR4 (e.g., mogamulizumab); CCR5 (e.g., Ieronlimab); CCR8; CD3 [e.g. blinatumomab (CD3/CD19 bispecific), PF-06671008 (CD3/P-cadherin bispecific), PF-06863135 (CD3/BCMA bispecific), CD25; CD28; CD30 (e.g., brentuximab vedotin); CD33 (e.g. gemtuzumab ozogamicin); CD38 (e.g., daratumumab, isatuximab), CD44v6; CD63; CD79 (e.g., polatuzumab vedotin); CD80; CD123; CD276/B7-H3 (e.g., omburtamab); CDH17; CEA; ClhCG; desmoglein 4; DLL3 (e.g., rovalpituzumab tesirine); DLL4; E-cadherin; EDA; EDB; EFNA4; EGFR (e.g., cetuximab, depatuxizumab mafodotin, necitumumab, panitumumab); EGFRvIII; Endosialin; EpCAM (e.g., oportuzumab monatox); FAP; Fetal Acetylcholine Receptor; FLT3 (e.g. see WO2018/220584); GD2 (e.g., dinutuximab, 3F8); GD3; GloboH; GMl; GM2; HER2/neu [e.g., margetuximab, pertuzumab, trastuzumab; ado-trastuzumab emtansine, trastuzumab duocarmazine, PF-06804103 (see U.S. Pat. No. 8,828,401)]; HER3; HER4; ICOS; ITG-AvB6; LAG-3 (e.g., relatlimab); Lewis-Y; LG; Ly-6; M-CSF [e.g. PD-0360324 (see U.S. Pat. No. 7,326,414)]; MCSP; mesothelin; MUCl; MUC2; MUC3; MUC4; MUCSAC; MUCSB; MUC7; MUC16; Notchl; Notch3; Nectin-4 (e.g. enfortumab vedotin); P-Cadherein [e.g. PF-06671008 (see WO2016/001810)]; PCDHB2; PDGFRA (e.g. olaratumab); Plasma Cell Antigen; PolySA; PSCA; PSMA; PTK7 [e.g. PF-06647020 (see, U.S. Pat. No. 9,409,995)]; Rorl; SAS; SCRx6; SLAMF7 (e.g. elotuzumab); SHH; SIRPa (e.g., ED9, Effi-DEM); STEAP; TGF-beta; TIGIT; TMPRSS3; TNF-alpha precursor; TROP-2 (e.g., sacituzumab govitecan); TSPAN8; and Wue-1. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g., calicheamicin, especially calicheamicin gammall and calicheamicin phill, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above. Also included are antihormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LYll 7018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and antiandrogens such as flutamide, nilutamide, bicalutamide, leuprolide, fluridil, apalutamide, enzalutamide, cimetidine and goserelin; KRAS inhibitors; MCT4 inhibitors; MAT2a inhibitors; tyrosine kinase inhibitors such as sunitinib, axitinib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600; erb inhibitors such as dacomitinib; PARP inhibitors such as talazoparib; SMO inhibitors such as glasdegib, PF-5274857; EGFR T790M inhibitors such as PF-06747775; EZH2 inhibitors such as PF-06821497; PRMT5 inhibitors such as PF-06939999; TGFR-rl inhibitors such as PF-06952229; and pharmaceutically acceptable salts, acids or derivatives of any of the above.

In some embodiments, an IL-21 variant or an IL-21 fusion protein is used in conjunction with one or more other therapeutic agents targeting an immune checkpoint modulator, such as, for example without limitation, an agent targeting PD-1, PDL-1, CTLA-4, LAG-3, B7-H3, B7-H4, B7-DC (PD-L2), B7-H5, B7-H6, B7-H8, B7-H2, B7-1, B7-2, ICOS, ICOS-L, TIGIT, CD2, CD47, CD80, CD86, CD48, CD58, CD226, CD155, CD112, LAIR1, 2B4, BTLA, CD160, TIMI, TIM-3, TIM4, VISTA (PD-Hl), OX40, OX40L, GITRL, CD70, CD27, 4-18B, 4-BBL, DR3, TLlA, CD40, CD40L, CD30, CD30L, LIGHT, HVEM, SLAM (SLAMFl, CD150), SLAMF2 (CD48), SLAMF3 (CD229), SLAMF4 (2B4, CD244), SLAMF5 (CD84), SLAMF6 (NTB-A), SLAMCF7 (CS!), SLAMF8 (BLAME), SLAMF9 (CD2F), CD28, CEACAM1(CD66a), CEACAM3, CEACAM4, CEACAM5, CEACAM6, CEACAM7, CEACAM8, CEACAM1-3AS CEACAM3C2, CEACAMl-15, PSGl-11, CEACAM1-4C1, CEACAM1-4S, CEACAM1-4L, IDO, TDO, CCR2, CD39-CD73-adenosine pathway (A2AR), BTKs, TIKs, CXCR2, CCR4, CCR8, CCR5, VEGF pathway, CSF-1, or an innate immune response modulator.

In some embodiments, an IL-21 variant or an IL-21 fusion protein is used in conjunction with a biotherapeutic agent and a chemotherapeutic agent. For example, this disclosure provides methods for treating cancer in a subject in need thereof that include administering to the subject an effective amount of the IL-21 variant or IL-21 fusion protein as described herein, a therapeutic antibody, and a chemotherapeutic agent (e.g., gemcitabine, methotrexate, or a platinum analog).

In some embodiments, the disclosure provides methods for treating cancer in a subject in need thereof including administering to the subject an effective amount, e.g., therapeutically effective amount, of the IL-21 variant or IL-21 fusion protein as described wherein, a therapeutic antibody (e.g., nivolumab (OPDIVO®), mAb7 (e.g., as described in US Pub. No. US20160159905, hereby incorporated by reference), or pembrolizumab (KEYTRUDA®), and a chemotherapeutic agent (e.g., gemcitabine, methotrexate, or a platinum analog). In some embodiments, methods are provided for treating cancer in a subject in need thereof comprising administering to the subject an effective amount, e.g., therapeutically effective amount, of the IL-21 variant or IL-21 fusion proteins as described wherein, an anti-CTLA-4 antagonist antibody (e.g., ipilimumab (YERVOY®)), and a chemotherapeutic agent (e.g., gemcitabine, methotrexate, or a platinum analog).

In some embodiments, the IL-21 variant or IL-21 fusion protein therapy may precede or follow the other agent treatment by intervals ranging from minutes to weeks. In embodiments where the other agents and/or a proteins or polynucleotides are administered separately, one would generally ensure that a significant period of time did not expire between each delivery, such that the agent and the composition of the present invention would still be able to exert an advantageously combined effect on the subject. In such instances, it is contemplated that one may administer both modalities within about 12-24 hours of each other, e.g., within about 6-12 hours of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

In some embodiments, an IL-21 variant or an IL-21 fusion protein composition comprises a second agent selected from crizotinib, palbociclib, gemcitabine, cyclophosphamide, fluorouracil, FOLFOX, folinic acid, oxaliplatin, axitinib, sunitinib malate, tofacitinib, bevacizumab, rituximab, and trastuzumab.

In some embodiments, an IL-21 variant or IL-21 fusion protein composition is combined with a treatment regimen further comprising a traditional therapy selected from the group consisting of: surgery, radiation therapy, chemotherapy, targeted therapy, immunotherapy, hormonal therapy, angiogenesis inhibition and palliative care.

### Formulations

Therapeutic formulations of the IL-21 variant or IL-21 fusion protein used in accordance with the present disclosure are prepared for storage by mixing the protein having the desired degree of purity with optional pharmaceutically acceptable carriers, excipients, or stabilizers (Remington, The Science and Practice of Pharmacy 20th Ed. Mack Publishing, 2000), e.g., in the form of lyophilized formulations or aqueous solutions. Acceptable carriers, excipients, or stabilizers are nontoxic to recipients at the dosages and concentrations employed, and may include buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Liposomes containing the IL-21 variant or IL-21 fusion protein are prepared by methods known in the art, such as described in Epstein, et al., Proc. Natl. Acad. Sci. USA 82:3688 (1985); Hwang, et al., Proc. Natl Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. The active ingredients may also be entrapped in microcapsules prepared, for example, by coacervation techniques or by interfacial polymerization, for example, hydroxymethylcellulose or gelatin-microcapsules and poly(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy 20th Ed. Mack Publishing (2000).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, nondegradable ethylene-vinyl acetate, degradable lactic acid glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic IL-21 variant or IL-21 fusion protein compositions are generally placed into a container having a sterile access port, for example, an intravenous solution bag or vial having a stopper pierceable by a hypodermic injection needle.

The compositions according to the present invention may be in unit dosage forms such as tablets, pills, capsules, powders, granules, solutions or suspensions, or suppositories, for oral, parenteral, or rectal administration, or administration by inhalation or insufflation.

For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. Suitable surface-active agents include, in particular, non-ionic agents, such as polyoxyethylene sorbitans (e.g., Tween™ 20, 40, 60, 80 or 85) and other sorbitans (e.g., Span™ 20, 40, 60, 80 or 85). Compositions with a surface-active agent will conveniently include between 0.05 and 5% surface-active agent, e.g., between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Jnfonutrol™, Lipofundin™, and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an IL-21 variant or IL-21 fusion protein with Intralipid™ or the components thereof (soybean oil, egg phospholipids, glycerol, and water). Compositions for inhalation or insuflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension, or powder compositions may be administered, preferably orally or nasally, from devices which deliver the formulation in an appropriate manner. Kits

The disclosure also provides kits comprising any or all of the IL-21 variants or IL-21-fusion proteins described herein. Kits of the invention include one or more containers comprising an IL-21 variant or IL-21 fusion protein described herein and instructions for use in accordance with any of the methods of the invention described herein. Generally, these instructions comprise a description of administration of the IL-21 variant or IL-21 fusion protein for the therapeutic treatments described herein. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of an IL-21 variant or an IL-21 fusion protein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an IL-21 variant or an IL-21 fusion protein. The container may further comprise a second pharmaceutically active agent.

Kits may optionally provide additional components such as buffers and interpretive information. Normally, the kit comprises a container and a label or package insert(s) on or associated with the container.

### EXAMPLES

The invention is further described in the following examples, which do not limit the scope of the invention described in the claims.

### Example 1: Validating IL-21/IL-21R Interaction Network Using AlphaSeq™

Prior to measuring binding affinities of IL-21 variants using the IL-21R receptor, the wild-type IL-21 and IL-21R interactions were validated using the AlphaSeq™ assay. Various construct designs for human, cynomolgus monkey (cyno), mouse, and rat IL-21 and IL-21R were designed and constructed into AlphaSeq™ yeast strains. An AlphaSeq™ assay was performed to measure all the pairwise interactions between IL-21 and IL-21R construct designs.

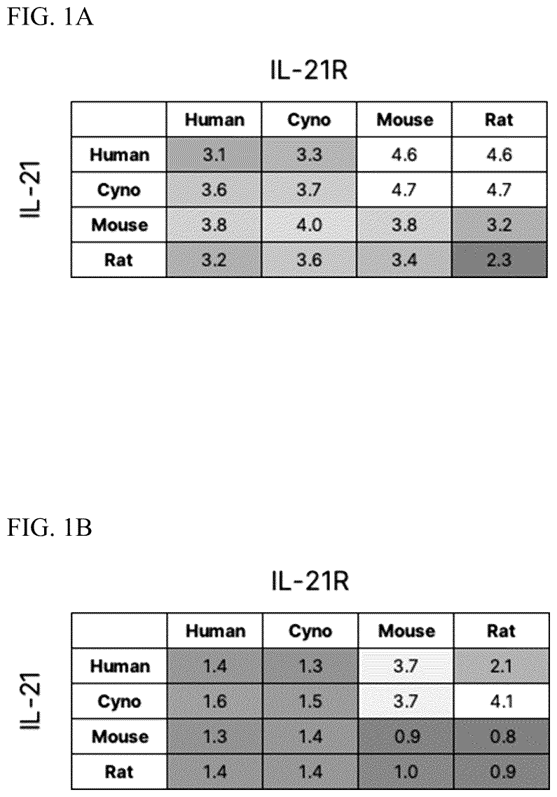

AlphaSeq™ predicted affinities (log 10K_{d} nM) for the IL-21/IL-21R validation network under standard conditions are shown in FIG. 1A. Strong binding is considered below 3 (dark gray), weak binding is considered between 3 and 4.5 (medium gray), no binding is considered above 4 (lightest gray). In standard conditions, human and cyno IL-21 each bound to both human and cyno IL-21R, while mouse and rat IL-21 each bound to all four IL-21R orthologs. Only the rat IL-21/ratIL-21R showed strong binding (AlphaSeq™ Kd<3)

AlphaSeq™ predicted affinities (log 10K_{d} nM) for the IL-21/IL-21R validation network were also measured in media buffered to pH 7 (FIG. 1B). In this condition, strong binding is considered below 2 (dark gray), weak binding is considered between 2 and 3.5 (medium gray), no binding is considered above 3.5 (lightest gray). Using these definitions, at pH 7, human IL-21 bound strongly to human and cyno IL-21R and weakly to rat IL-21R. Cyno IL-21 bound strongly to human and cyno IL-21R. Mouse and rat IL-21 bound strongly to all IL-21 orthologs.

### Example 2: Measuring Affinity of IL-21 Variants to IL-21R

To identify IL-21 variants with decreased affinity for human IL-21R using AlphaSeq™, a site saturation mutagenesis (SSM) library of IL-21 was constructed, such that each of the 121 amino acid residues in the displayed protein, which consists of residues 32-152 of IL-21, was mutated to every other amino acid, excluding cysteine. The variant library consisted of 2106 total variant IL-21 proteins, which was combined with 117 copies of the wild-type IL-21 sequence, such that the total library consisted of 2223 proteins. These 2223 proteins were synthesized and cloned into yeast display libraries and associated with nucleotide barcodes as described above. Each protein was displayed as a synthetic adhesion protein (SAP) fusion protein to the yeast agglutination factor Aga2 and the HA epitope tag.

To identify mouse IL-21 (mIL-21) variants with decreased affinity for mouse IL-21R (mIL-21R) using AlphaSeq™, a site saturation mutagenesis (SSM) library of mIL-21 was constructed, such that each of the amino acid residues in the displayed protein, which consists of residues 22-145 of mIL-21, was mutated to every other amino acid, excluding cysteine. The variant library consisted of 2160 total variant IL-21 proteins, which was combined with 120 copies of the wild-type IL-21 sequence, such that the total library consisted of 2280 proteins. These 2280 proteins were synthesized and cloned into yeast display libraries and associated with nucleotide barcodes as described above. Each protein was displayed as a synthetic adhesion protein (SAP) fusion protein to the yeast agglutination factor Aga2 and the HA epitope tag.

Using AlphaSeq™, affinity predictions were obtained for interactions between each of the human and mouse IL-21 SSM variants and human, cyno, mouse, and rat IL-21R. Variants of interest were those with decreased affinity (higher K_{d} values) than the wild-type IL-21/IL-21R interaction. Because an increased dynamic range was observed for IL-21/IL-21R interaction affinity when the AlphaSeq™ assay was performed in buffered media, IL-21/IL-21R binding was measured using both standard AlphaSeq™ conditions (unbuffered media), as well as media buffered at pH 7, pH 6, and pH 5. In the AlphaSeq™ assay, if a given variant has apparently decreased affinity, this could be due to true detuning—i.e., the affinity between IL-21 and IL-21R is actually decreased—or from a decrease in expression or stability of the particular IL-21 variant relative to wild-type. For therapeutic applications, the former is preferred. Thus, a parallel assay was performed to measure the surface expression of each displayed IL-21 variant.

To measure surface expression, the library of variants was labeled with a FITC-conjugated anti-HA antibody. Labeled cells were then sorted by FACS into one of three bins—low (bin 1), medium (bin 2), or high (bin 3)—based on fluorescence intensity. Cells expressing higher levels of IL-21 were expected to be enriched in the high bin and vice versa for those cells expressing lower levels of IL-21. DNA was extracted from each of the three sorted samples and subjected to deep Illumina™ sequencing at the barcode locus. The number of reads aligning to each barcode was counted for each bin and normalized to the total number of reads in that bin and to the number of reads in the unsorted library, which results in an enrichment score for each barcode in each bin.

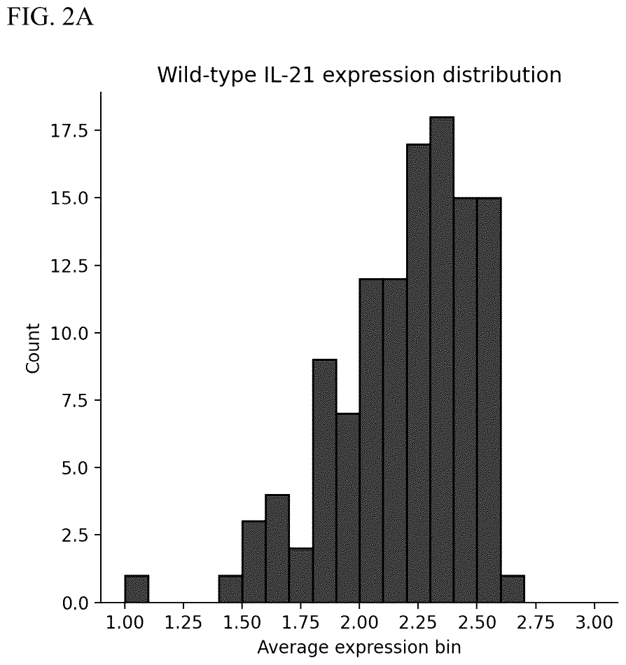

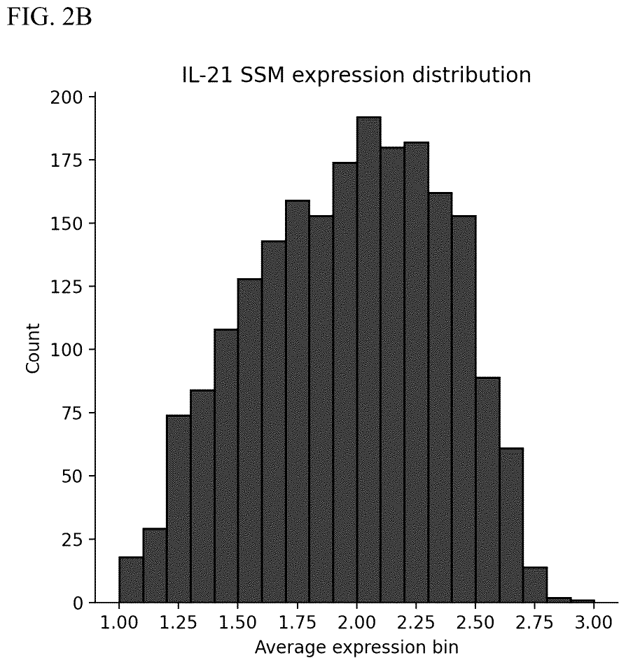

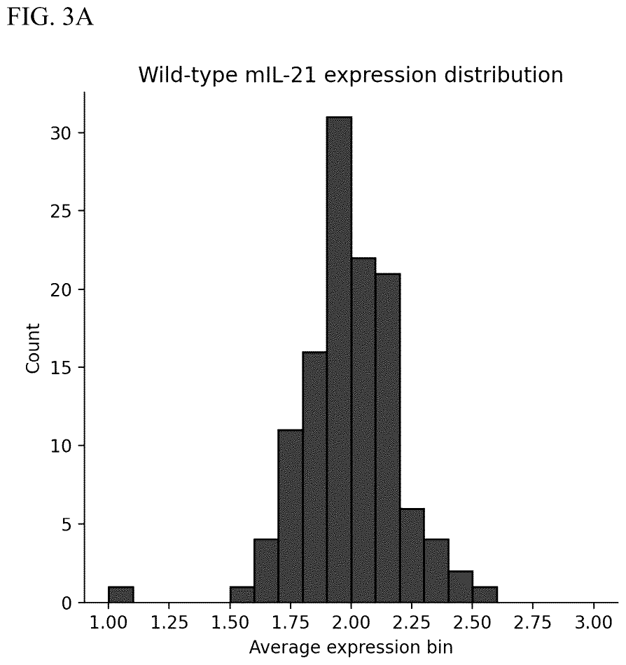

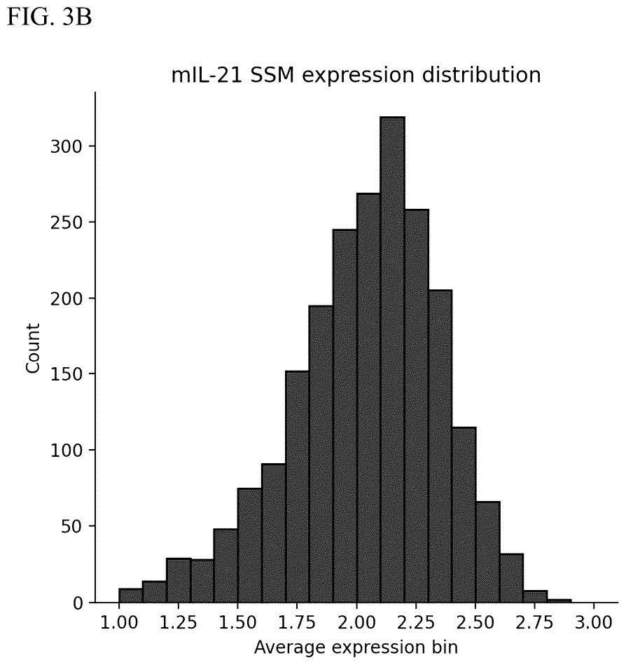

The enrichment scores for the 3 bins were combined into an expression value using the formula (1*bin1_enrichment+2*bin2_enrichment+3*bin3_enrichment)/(bin1_enrichment+bin2_enrichment+bin3_enrichment). This expression metric is termed the average expression bin for each barcode and returns a value between 1 and 3. Very highly expressing variants (i.e., those with most barcode reads in bin 3) have an average expression bin value approaching 3, while very lowly expressing variants (i.e., those with most reads in bin 1) have a value approaching 1. The average expression bin values for the various barcodes associated with each single IL-21 variant were combined as a weighted average based on frequency in the unsorted population to obtain a value for each protein. The distribution of average expression bin values for the wild-type copies of human IL-21 are shown in FIG. 2A and the values for the members of the human IL-21 SSM library are shown in FIG. 2B. The distribution of average expression bin values for the wild-type copies of mouse IL-21 are shown in FIG. 3A and the values for the members of the mouse IL-21 SSM library are shown in FIG. 3B.

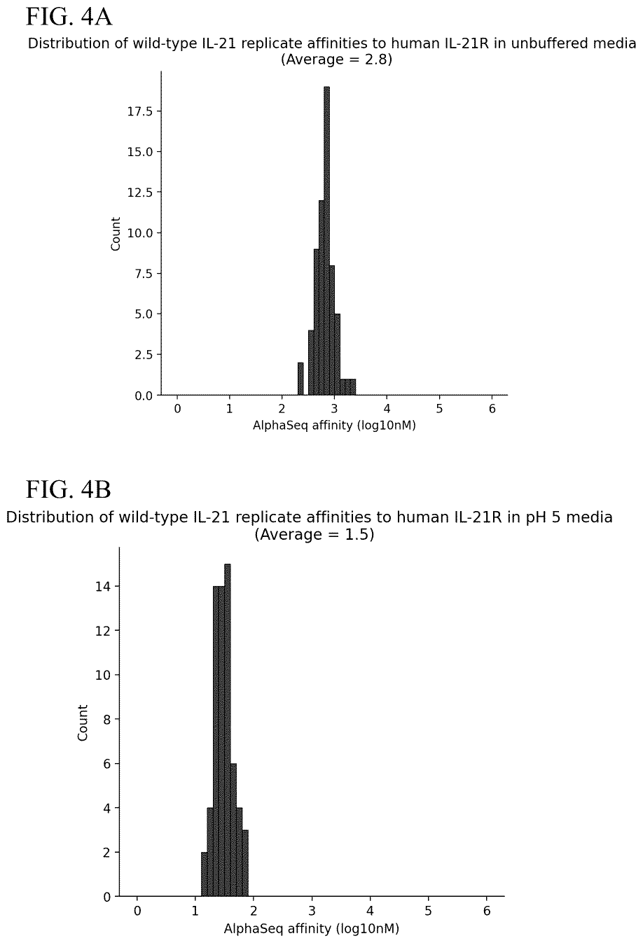

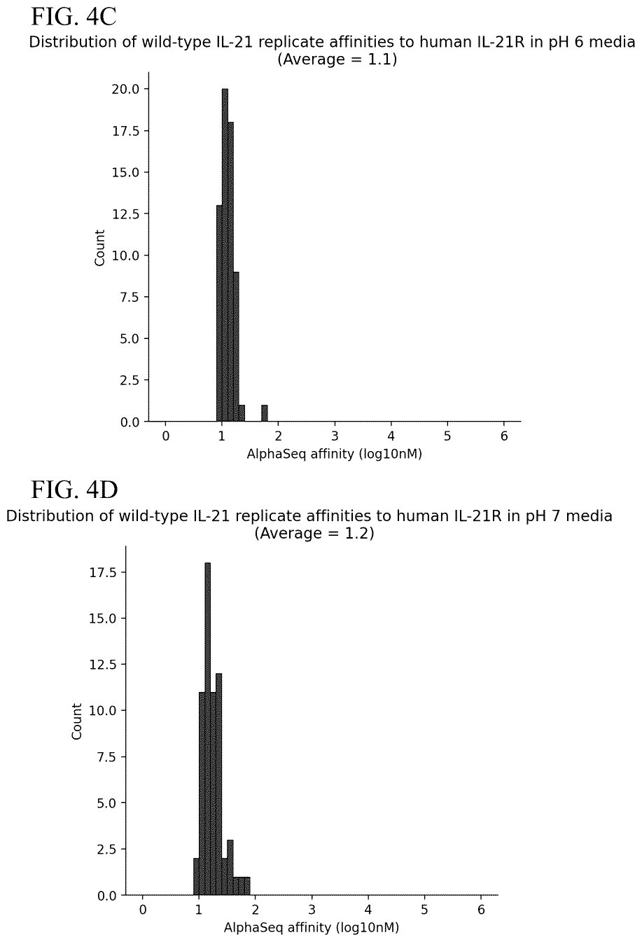

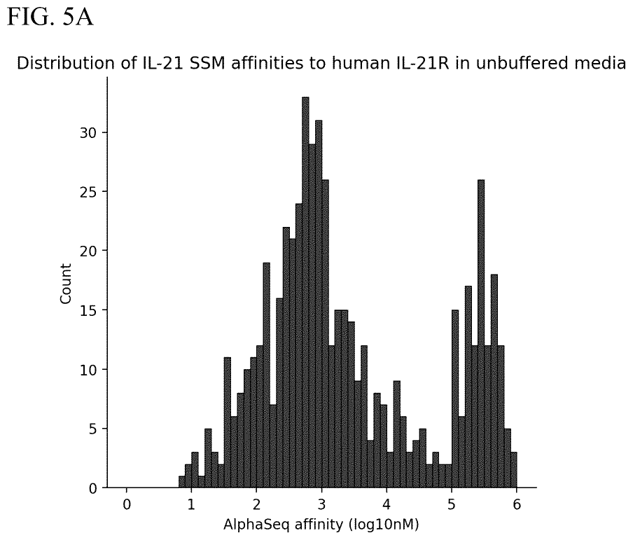

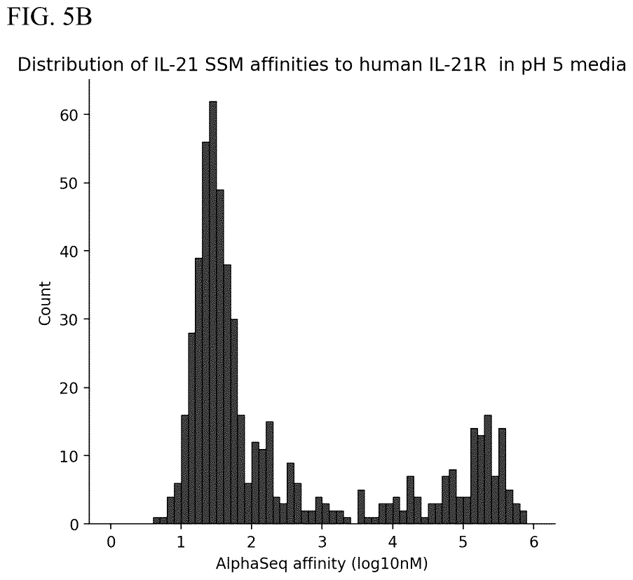

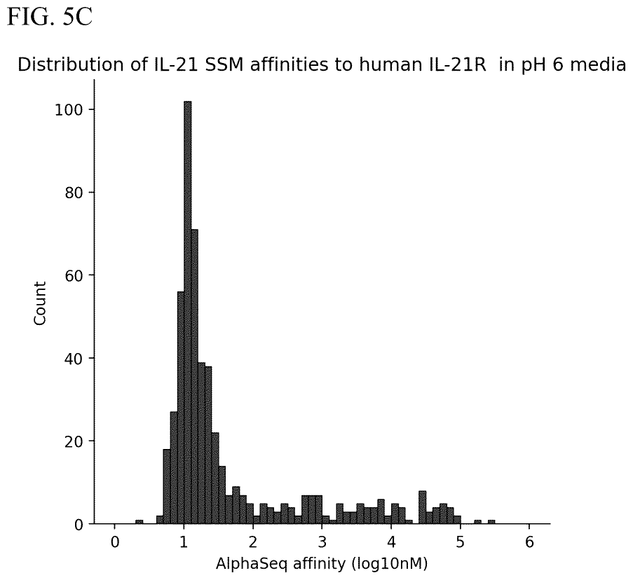

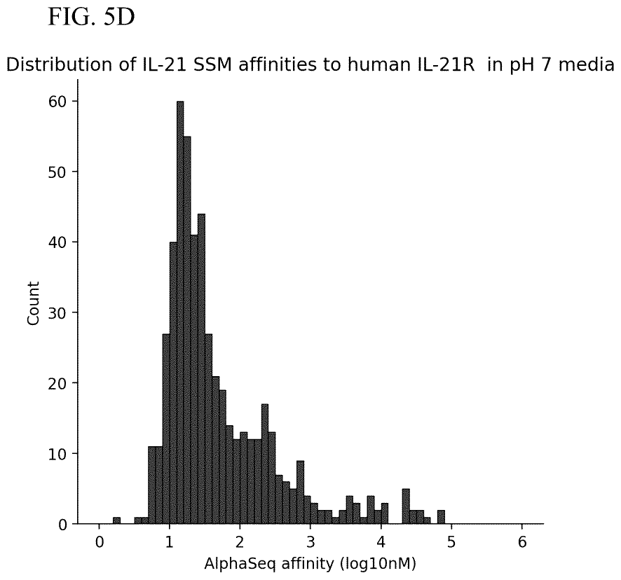

From these datasets, for each human IL-21 variant, an affinity for human IL-21R was obtained and an expression score was measured. After filtering AlphaSeq™ results to include only variants with average expression bin>2.25, the average affinity between the 117 wild-type IL-21 replicates and human IL-21R was found to be 646 nM in unbuffered media (log 10 nM=2.8 in FIG. 4A), 30 nM in pH 5 media (log 10 nM=1.5 in FIG. 4B), 13 nM in pH 6 media (log 10 nM=1.1 in FIG. 4C), and 18 nM in pH 7 media (log 10 nM=1.2 in FIG. 4D). The distribution of IL-21 SSM mutant affinities to human IL-21R are shown in unbuffered media (FIG. 5A), pH 5 media (FIG. 5B), pH 6 media (FIG. 5C), and pH 7 media (FIG. 5D). In contrast to the wild-type affinity values, which show a tight distribution, the IL-21 variants show a wide range of affinities, spanning from wild-type levels of binding to background-level binding. The minimal criteria for considering a variant detuned were as follows: expression>2.25 on a scale where no expression=1.0 and maximum expression=3.0; affinity measurements representing a half-log reduction in binding affinity from wild-type human IL-21 in at least one of the tested media conditions. This value varied depending on the media condition used:
Unbuffered detuned variant cutoff: 2.0 μM (log 10 nM=3.3)
pH 7: 56 nM (log 10 nM=1.7)
pH 6: 41 nM (log 10 nM=1.6)
pH 5: 94 nM (log 10 nM=2.0)
   These criteria lead to a total of 273 IL-21 variants (Table 1), of which 194 retain binding affinity stronger than the approximate assay limit of detection (limit of detection defined as 2 standard deviations stronger affinity than the mean of negative control interactions).

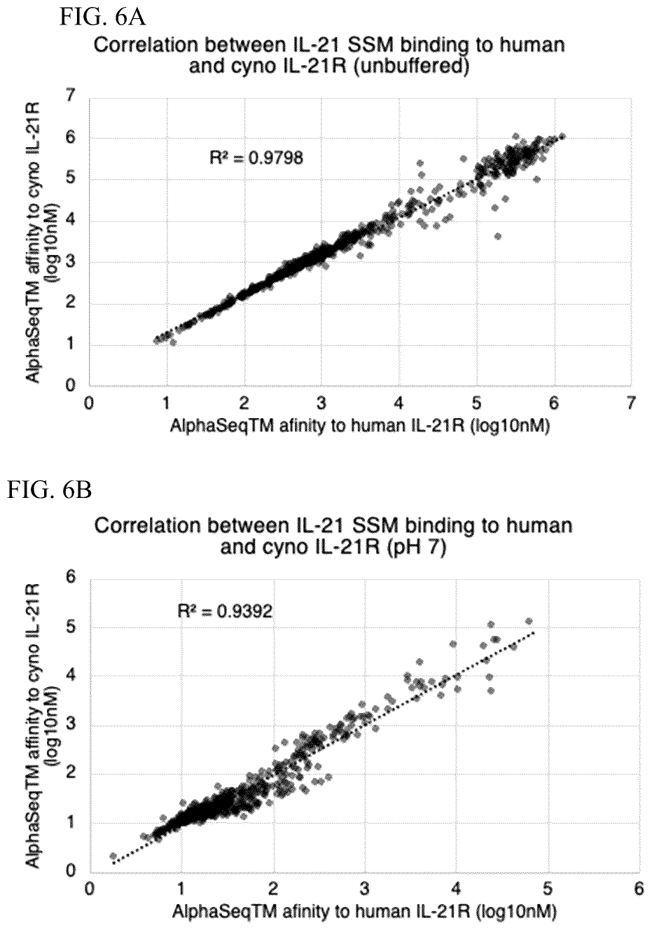

The AlphaSeq™ platform allowed simultaneous measurement of IL-21 variant affinities to cyno, rat, and mouse orthologs of IL-21R. Human IL-21 variants showed highly correlated degrees of detuning to human and cyno IL-21R in both unbuffered media (FIG. 6A) and pH 7 media (FIG. 6B).

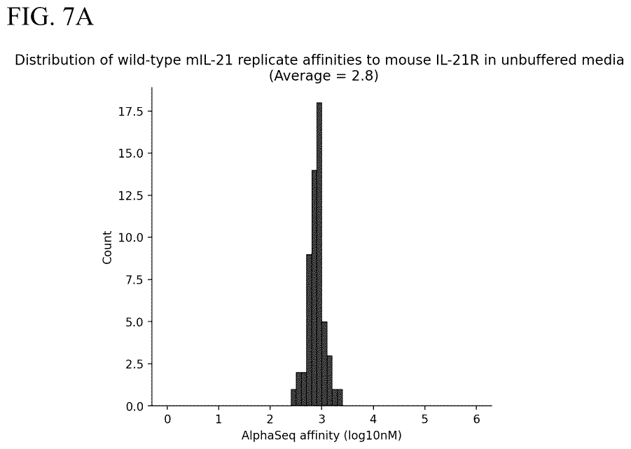

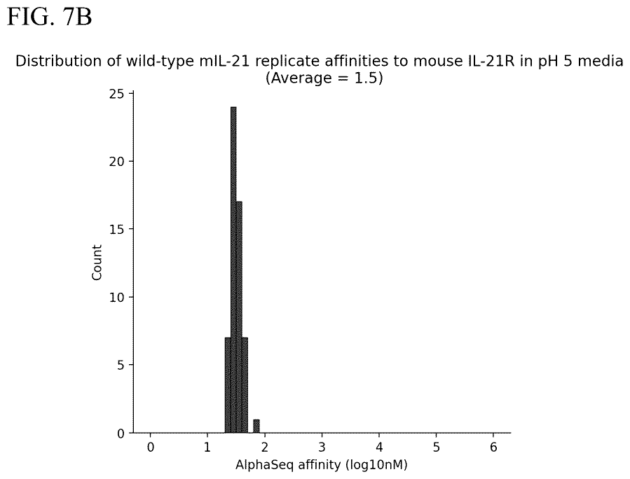

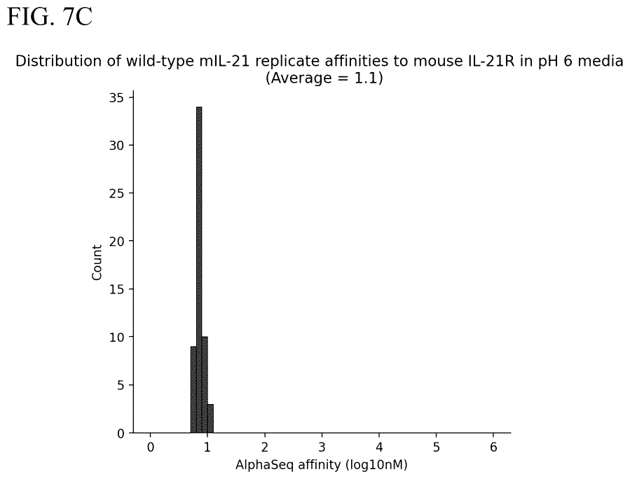

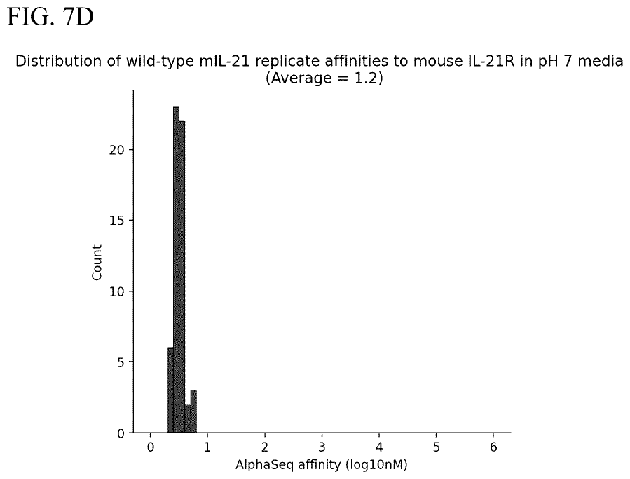

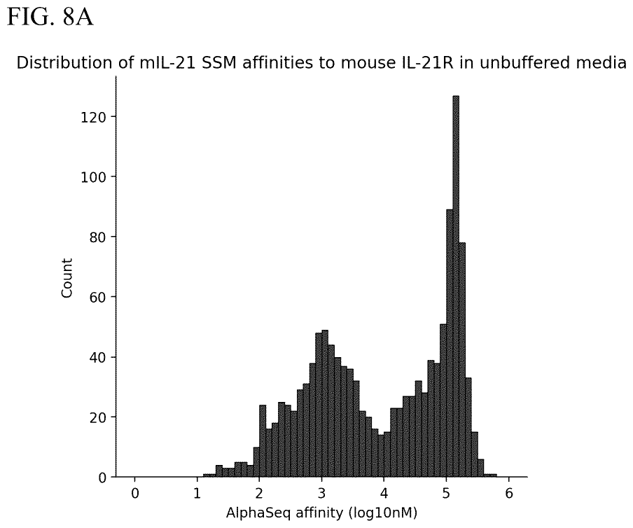

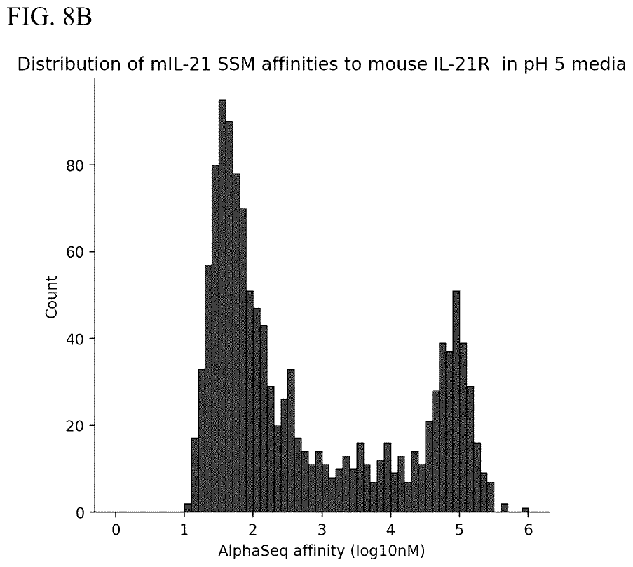

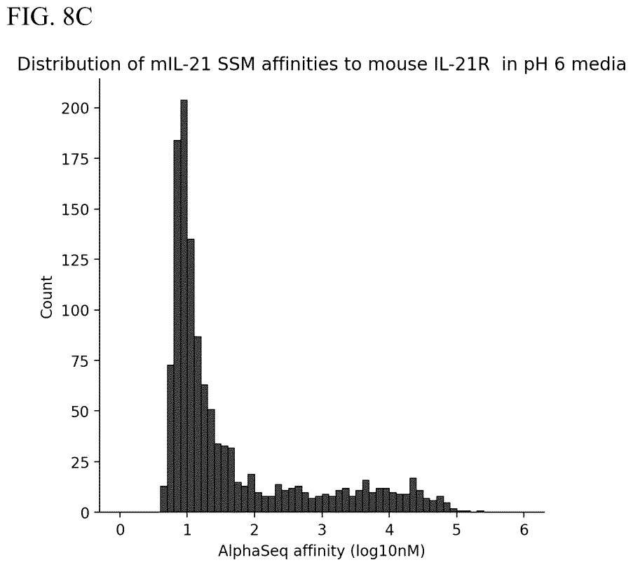

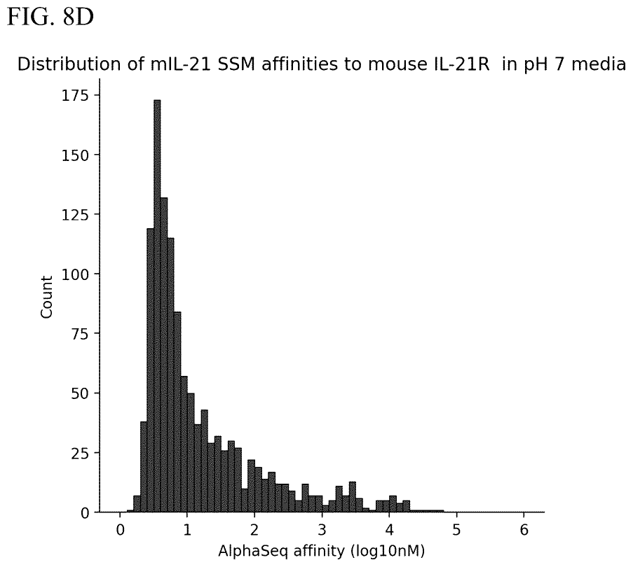

For each mIL-21 variant, an affinity for mouse IL-21R was obtained and an expression score was measured. After filtering AlphaSeq™ results to include only variants with average expression bin>2.0, the average affinity between the 120 wild-type mIL-21 replicates and mouse IL-21R was found to be 777 nM in unbuffered media (log 10 nM=2.9 in FIG. 7A), 32 nM in pH 5 media (log 10 nM=1.5 in FIG. 7B), 7.3 nM in pH 6 media (log 10 nM=0.9 in FIG. 7C), and 3.1 nM in pH 7 media (log 10 nM=0.5 in FIG. 7D). The distribution of mIL-21 SSM mutant affinities to mouse IL-21R are shown in unbuffered media (FIG. 8A), pH 5 media (FIG. 8B), pH 6 media (FIG. 8C), and pH 7 media (FIG. 8D). In contrast to the wild-type affinity values, which show a tight distribution, the mIL-21 variants show a wide range of affinities, spanning from wild-type levels of binding to background-level binding. The minimal criteria for considering a mouse variant detuned were as follows: expression>2.0 on a scale where no expression=1.0 and maximum expression=3.0; affinity measurements representing a half-log reduction in binding affinity from wild-type mouse IL-21 in at least one of the tested media conditions. This value varied depending on the media condition used:
Unbuffered detuned variant cutoff: 2.5 μM (log 10 nM=3.4)
pH 7: 10 nM (log 10 nM=1.0)
pH 6: 23 nM (log 10 nM=1.4)
pH 5: 101 nM (log 10 nM=2.0)
   These criteria lead to a total of 812 mIL-21 variants (Table 3), of which 690 retain binding affinity above the approximate limit of detection of the assay (limit of detection defined as 2 standard deviations stronger affinity than the mean of negative control interactions).

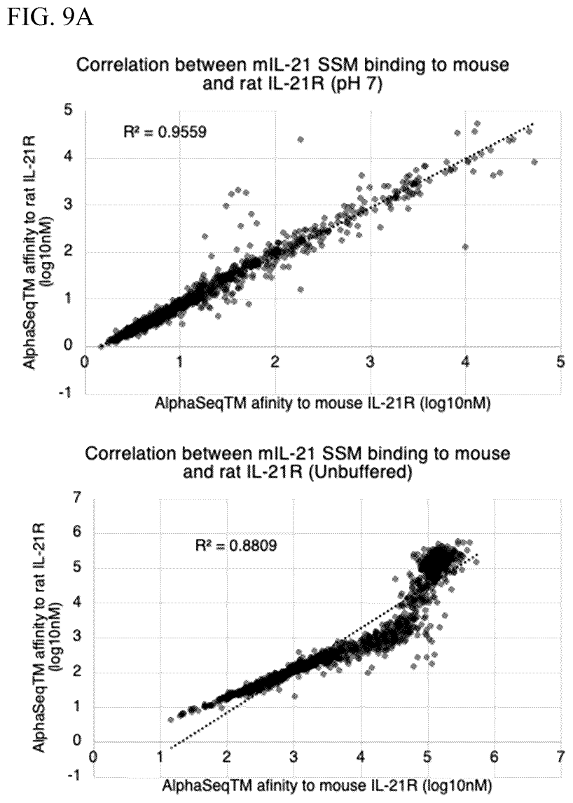

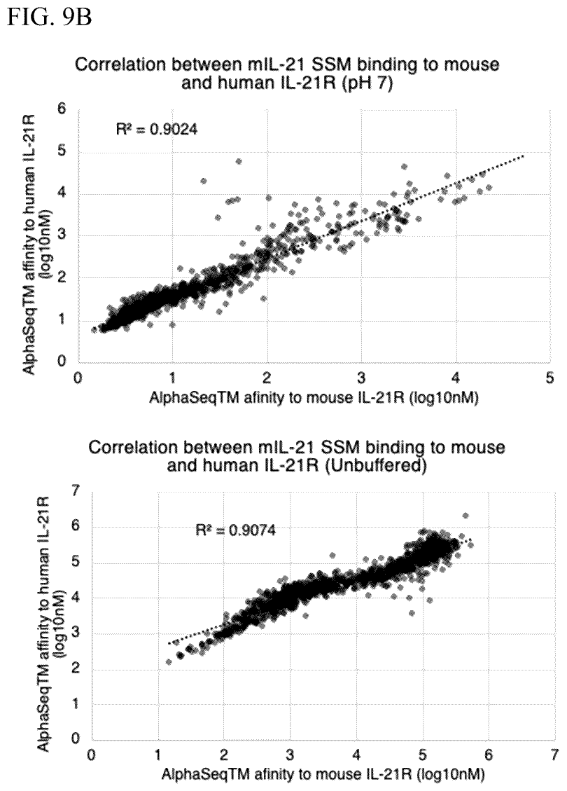

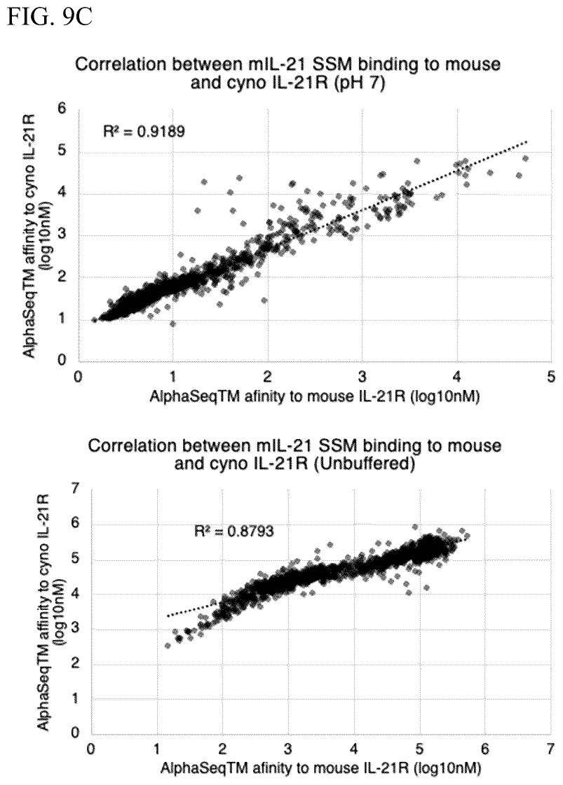

The AlphaSeq™ platform allowed simultaneous measurement of mIL-21 variant affinities to cyno, rat, and mouse orthologs of IL-21R. Mouse IL-21 variant detuning to mouse IL-21R was highly correlated with the degree of detuning to rat IL-21R (FIG. 9A), human IL-21R (FIG. 9B), and cyno IL-21R (FIG. 9C).

The results for screening of interaction between human IL-21 variants and human IL-21R under each condition are provided in Table 1. The results for screening of interaction between mouse IL-21 variants and mouse IL-21R under each condition are provided in Table 3.

Further selection criteria were applied to the 273 human IL-21 variants and 812 mouse IL-21 variants identified as being detuned for prioritizing candidate molecules for further functional characterization. Those criteria included: formation of ≥50 diploids (unbuffered) or ≥10 diploids (buffered) in the AlphaSeq™ assay; identification of multiple substitutions at the same position that lead to detuning (termed the “multiplicity” for that position); diversity of positions on the IL-21 structure; amino acid conservation across human, cyno, mouse, and rat IL-21; conserved detuned positions between human and mouse IL-21. Using these criteria, a list of 35 human IL-21 and 36 mouse IL-21 variants were selected to express as soluble proteins for biochemical and functional characterization (Table 4).

TABLE 4
Characterizing Prioritized Human IL-21 Variants as Soluble Fc
Fusion Proteins for Binding K_{d} Values and Potency EC50 Values

| | IL21RA_human | IL21RA_mouse | Human T | Mouse T |
|---|---|---|---|---|
| | Kd, BLI | Kd, BLI | cell EC50 | cell EC50 |
| variant | (M) | (M) | (pM) | (pM) |
| IL-21_human_WT | 1.48E−09 | 5.47E−09 | 1.3E+00 | Not tested |
| IL-21_human_D33P | 9.03E−09 | 1.98E−08 | 9.3E+00 | 1.8E+03 |
| IL-21_human_S74H | 1.23E−08 | 1.93E−08 | 3.6E+00 | 1.9E+03 |
| IL-21_human_Y52Q | 1.44E−08 | 1.94E−08 | 8.7E+00 | 9.2E+03 |
| IL-21_human_W73L | 2.28E−08 | 1.99E−08 | 1.5E+01 | 6.9E+03 |
| IL-21_human_V53T | 7.86E−09 | 5.09E−09 | Not tested | Not tested |
| IL-21_human_R34S | 3.15E−08 | 2.87E−08 | 5.6E+00 | 8.5E+03 |
| IL-21_human_P108L | 3.21E−09 | 1.18E−08 | 1.1E+00 | 7.1E+04 |
| IL-21_human_I45A | 1.94E−09 | 1.26E−08 | 1.3E+00 | NA |
| IL-21_human_E72S | 9.65E−09 | 7.75E−09 | 3.1E+01 | 4.7E+04 |
| IL-21_human_I37V | 3.02E−09 | 5.27E−09 | Not tested | Not tested |
| IL-21_human_F76M | 1.18E−08 | NA | Not tested | Not tested |
| IL-21_human_I43M | 7.02E−09 | 9.04E−09 | Not tested | Not tested |
| IL-21_human_F76E | NA | NA | Not tested | Not tested |
| IL-21_human_R38K | 3.13E−07 | 2.62E−07 | Not tested | Not tested |
| IL-21_human_R38Q | 8.96E−08 | 2.46E−07 | Not tested | Not tested |
| IL-21_human_F79P | 1.11E−08 | NA | Not tested | Not tested |
| IL-21_human_R38M | 8.79E−08 | 1.89E−07 | Not tested | Not tested |
| IL-21_human_D44P | 1.36E−06 | NA | Not tested | Not tested |
| IL-21_human_K106S | 5.78E−09 | 2.40E−08 | Not tested | Not tested |
| IL-21_human_I45G | 1.20E−08 | NA | Not tested | Not tested |
| IL-21_human_V53N | 8.71E−08 | NA | Not tested | Not tested |
| IL-21_human_W73V | 6.30E−09 | 1.06E−07 | Not tested | Not tested |
| IL-21_human_R34D | 6.21E−08 | 1.03E−07 | Not tested | Not tested |
| IL-21_human_I37G | 6.17E−08 | 1.59E−07 | Not tested | Not tested |
| IL-21_human_I45H | 2.90E−08 | 1.24E−07 | Not tested | Not tested |
| IL-21_human_Y52D | 9.30E−09 | 2.70E−07 | Not tested | Not tested |
| IL-21_human_I95H | 1.60E−08 | 8.49E−07 | Not tested | Not tested |
| IL-21_human_P108G | 2.90E−08 | NA | Not tested | Not tested |
| IL-21_human_L42Q | 6.09E−08 | NA | Not tested | Not tested |
| IL-21_human_I148S | 2.45E−09 | 6.46E−09 | Not tested | Not tested |
| IL-21_human_D33T | 1.48E−08 | 3.74E−08 | Not tested | Not tested |
| IL-21_human_R105K | 1.67E−08 | 4.13E−08 | Not tested | Not tested |
| IL-21_human_I37R | 9.97E−09 | 3.51E−08 | Not tested | Not tested |
| IL-21_human_I45D | NA | NA | Not tested | Not tested |
| IL-21_human_E72L | 4.40E−09 | 1.23E−08 | Not tested | Not tested |
| IL-21_mouse_WT | 6.38E−09 | 5.08E−09 | Not tested | 6.7E+00 |
| IL-21_mouse_D26P | NA | 4.02E−08 | Not tested | 2.6E+01 |
| IL-21_mouse_P101L | NA | NA | Not tested | 2.5E+03 |
| IL-21_mouse_R27S | 2.22E−08 | 1.09E−08 | Not tested | 1.8E+01 |
| IL-21_mouse_I30V | 8.86E−09 | 6.15E−09 | Not tested | 5.8E+00 |
| IL-21_mouse_Y45Q | 8.37E−09 | 7.05E−09 | Not tested | 8.2E+01 |
| IL-21_mouse_I38A | 4.61E−09 | 6.61E−09 | Not tested | 1.4E+02 |
| IL-21_mouse_F88D | 5.94E−09 | 3.26E−09 | Not tested | 5.7E+00 |
| IL-21_mouse_F69M | 9.19E−09 | 5.65E−09 | Not tested | 6.7E+00 |
| IL-21_mouse_R99L | 1.05E−08 | 6.59E−09 | Not tested | 2.0E+01 |
| IL-21_mouse_Q95S | 9.35E−09 | 6.61E−09 | Not tested | 2.9E+01 |
| IL-21_mouse_R97V | NA | NA | Not tested | 1.4E+02 |
| IL-21_mouse_L96M | 4.52E−09 | 6.11E−09 | Not tested | 7.6E+01 |
| IL-21_mouse_F72R | NA | NA | Not tested | NA |
| IL-21_mouse_L32R | NA | NA | Not tested | NA |
| IL-21_mouse_P57E | 1.05E−07 | 2.16E−08 | Not tested | NA |
| IL-21_mouse_A56D | 7.32E−09 | 4.77E−09 | Not tested | NA |
| IL-21_mouse_I38D | 4.58E−09 | 0.00E+00 | Not tested | 1.39E+04 |
| IL-21_mouse_L92E | 3.54E−09 | 1.04E−08 | Not tested | 1.47E+04 |
| IL-21_mouse_L130W | NA | 1.20E−08 | Not tested | NA |
| IL-21_mouse_I38G | NA | NA | Not tested | NA |
| IL-21_mouse_L35K | NA | NA | Not tested | 1.14E+04 |
| IL-21_mouse_F69H | NA | 1.33E−09 | Not tested | 2.63E+04 |
| IL-21_mouse_N85I | NA | NA | Not tested | 5.25E+03 |
| IL-21_mouse_P101G | 4.39E−09 | NA | Not tested | 6.55E+02 |
| IL-21_mouse_L100S | 1.99E−07 | 8.71E−09 | Not tested | 1.05E+04 |
| IL-21_mouse_V60K | 4.01E−08 | 1.40E−08 | Not tested | 4.42E+03 |
| IL-21_mouse_I89D | 3.19E−09 | 6.07E−09 | Not tested | 1.07E+04 |
| IL-21_mouse_R27D | 1.51E−07 | 9.08E−08 | Not tested | 3.45E+02 |
| IL-21_mouse_A68E | NA | 4.34E−09 | Not tested | 1.33E+04 |
| IL-21_mouse_V39Q | NA | NA | Not tested | NA |
| IL-21_mouse_D37P | 2.50E−09 | NA | Not tested | 1.46E+03 |
| IL-21_mouse_Y45D | 4.31E−09 | NA | Not tested | 5.10E+03 |
| IL-21_mouse_R98A | NA | 4.27E−08 | Not tested | 6.00E+01 |
| IL-21_mouse_I30R | NA | 1.35E−08 | Not tested | 1.00E+01 |
| IL-21_mouse_I141S | 4.72E−11 | 3.07E−09 | Not tested | 5.25E+02 |
| IL-21_mouse_D26T | NA | 6.85E−10 | Not tested | 3.50E+01 |

Note:
“Not tested” indicates that the protein was not tested by this assay. “NA” indicates that the protein was tested, but the value was outside the limit of detection of the asay.

### Example 3: Expression and Characterization of Detuned IL-21 Variant Fc Fusion Proteins

Cytokine variant sequences identified with appropriate AlphaSeq™ expression and detuned affinity were nominated for recombinant protein expression. Double stranded DNA fragments coding the identified variant sequence were synthesized by Twist Biosciences which included flanking sequences to facilitate cloning into the pCDNA3.1(+) mammalian cell expression vector. The commercial vector had been modified to encode the human IgG1 Fc sequence containing effectorless function mutations. Variant cytokine insert fragments were cloned into the modified expression vector using Gibson Assembly and transformed into host TOP10 E. Coli competent cells. Cytokine sequence of interest was verified by Sanger sequencing. Confirmed sequence plasmid DNA was isolated from bacteria cultures using commercial DNA miniprep kits. The ExpiCHO™ mammalian cell line was thawed and cultured according to manufacturer instructions. Transient transfection of ExpiCHO™ cells was performed with detuned cytokine Fc construct DNA according to ExpiCHO™ transfection kit specifications and cultured supernatants were harvested 7 days later. Cultured supernatants containing proteins of interest were passed through a Protein A column using an AKTA FPLC instrument and eluted with low pH buffer. Purified protein was formulated in 20 mM histidine at pH 6.0. Harvest titers and percent purity as measured by analytical size exclusion chromatography (ASEC) are shown in Table 5.

TABLE 5
Protein Expression Yields and Purity for IL-21-Fc Fusion Proteins

| Variant | Titer (ug/mL) | Percent main peak |
|---|---|---|
| IL-21_human_WT | 53.569 | 87.433 |
| IL-21_human_D33P | 47.613 | 91.88 |
| IL-21_human_S74H | 54.99 | 90.669 |
| IL-21_human_Y52Q | 61.543 | 92.491 |
| IL-21_human_W73L | 67.435 | 90.091 |
| IL-21_human_V53T | 23.608 | 87.441 |
| IL-21_human_R34S | 57.635 | 93.742 |
| IL-21_human_P108L | 100.476 | 91.886 |
| IL-21_human_I45A | 82.273 | 90.594 |
| IL-21_human_E72S | 10.972 | 42.467 |
| IL-21_human_I37V | 44.597 | 91.921 |
| IL-21_human_F76M | 10.769 | 80.814 |
| IL-21_human_I43M | 51.717 | 81.806 |
| IL-21_human_F76E | 140.103 | 91.836 |
| IL-21_human_R38K | 79.113 | 94.544 |
| IL-21_human_R38Q | 166.941 | 95.123 |
| IL-21_human_F79P | 84.973 | 89.576 |
| IL-21_human_R38M | 138.504 | 95.266 |
| IL-21_human_D44P | 155.63 | 91.363 |
| IL-21_human_K106S | 108.406 | 86.763 |
| IL-21_human_I45G | 13.134 | 85.701 |
| IL-21_human_V53N | 127.984 | 89.068 |
| IL-21_human_W73V | 102.27 | 89.812 |
| IL-21_human_R34D | 80.043 | 81.76 |
| IL-21_human_I37G | 68.353 | 90.082 |
| IL-21_human_I45H | 267.795 | 92.724 |
| IL-21_human_Y52D | 108.789 | 77.088 |
| IL-21_human_I95H | 108.818 | 87.131 |
| IL-21_human_P108G | 23.049 | 79.61 |
| IL-21_human_L42Q | 133.326 | 85.191 |
| IL-21_human_I148S | 8.69 | 52.633 |
| IL-21_human_D33T | 96.5 | 90.943 |
| IL-21_human_R105K | 137.531 | 90.406 |
| IL-21_human_I37R | 112.868 | 90.326 |
| IL-21_human_I45D | 196.65 | 94.388 |
| IL-21_human_E72L | 285.652 | 88.349 |
| IL-21_mouse_WT | 54.734 | 64.805 |
| IL-21_mouse_D26P | 15.761 | 76.741 |
| IL-21_mouse_P101L | 37.509 | 79.028 |
| IL-21_mouse_R27S | 48.447 | 81.367 |
| IL-21_mouse_I30V | 48.438 | 85.922 |
| IL-21_mouse_Y45Q | 43.971 | 81.276 |
| IL-21_mouse_I38A | 38.539 | 87.714 |
| IL-21_mouse_F88D | 55.471 | 67.994 |
| IL-21_mouse_F69M | 56.085 | 83.685 |
| IL-21_mouse_R99L | 37.203 | 77.646 |
| IL-21_mouse_Q95S | 36.764 | 70.976 |
| IL-21_mouse_R97V | 18.309 | 76.928 |
| IL-21_mouse_L96M | 24.517 | 72.24 |
| IL-21_mouse_F72R | 70.036 | 71.721 |
| IL-21_mouse_L32R | 16.845 | 88.081 |
| IL-21_mouse_P57E | 40.411 | 65.04 |
| IL-21_mouse_A56D | 48.387 | 60.11 |
| IL-21_mouse_I38D | 114.926 | 87.478 |
| IL-21_mouse_L92E | 97.558 | 68.283 |
| IL-21_mouse_L130W | 50.266 | 64.038 |
| IL-21_mouse_I38G | 110.045 | 92.937 |
| IL-21_mouse_L35K | 21.855 | 23.419 |
| IL-21_mouse_F69H | 54.216 | 50.297 |
| IL-21_mouse_N85I | 37.637 | 51.2 |
| IL-21_mouse_P101G | 48.553 | 51.112 |
| IL-21_mouse_L100S | 59.417 | 46.623 |
| IL-21_mouse_V60K | 69.441 | 52.916 |
| IL-21_mouse_I89D | 83.519 | 38.905 |
| IL-21_mouse_R27D | 82.515 | 40.281 |
| IL-21_mouse_A68E | 60.369 | 42.21 |
| IL-21_mouse_V39Q | 18.848 | 51.411 |
| IL-21_mouse_D37P | 72.93 | 82.457 |
| IL-21_mouse_Y45D | 73.758 | 81.658 |
| IL-21_mouse_R98A | 70.668 | 67.686 |
| IL-21_mouse_I30R | 101.043 | 49.887 |
| IL-21_mouse_I141S | 56.028 | 62.483 |
| IL-21_mouse_D26T | 55.221 | 52.216 |

The binding kinetics for IL-21 variant Fc fusion proteins were determined by bio-layer interferometry analysis on a Gator Prime instrument. This assay was performed by immobilizing commercially available recombinant human IL-21R His tag (Acro Biosystems) to anti-His biosensors (Gator). Protein A purified IL-21 variant Fc fusion proteins association to and dissociation from the immobilized IL-21R was observed at the same concentration for all variants observed. Binding of all human and mouse IL-21 variants were measured against both human and mouse IL-21R.

Specifically, anti-His probes were hydrated in kinetics buffer (lx PBS with 0.2% BSA and 0.02% Tween-20) for 10 minutes and IL-21R his tag antigen was immobilized to the anti-his probe for 180 seconds. Association was observed by placing probes with immobilized antigen into wells containing IL-21 variant Fc fusion proteins for 120 seconds. Dissociation was measured after transferring the biosensors into wells containing only kinetics buffer for 120 seconds.

All assay steps were performed with shaking at 1000 rpm at 30° C. Off rate (koff) analysis for this assay were determined using the Gator software provided from the manufacturer using data points collected from the first 20 seconds of dissociation. Results of the kinetic studies are presented in Table 4. Using bio-layer interferometry (BLI), the variants demonstrated a range of binding affinities to IL-21R, including strong cross-reactivity between human and mouse.

A subset of human IL-21 Fc fusion variants were tested for signaling potency in primary human PBMCs using a phosphorylated STAT3 flow cytometry assay (pSTAT3 phosflow). Previously frozen human PBMCs (BloodworksBio) were thawed, washed, and counted to determine cell number and viability. Cells were plated in round-well bottom 96 well tissue culture plates for a total of 200,000 cells per well in 150 uL media. Wild-type and variant IL-21 Fc fusion proteins were added at concentrations ranging from 1 pM to 1 nM, and the cells were cultured for 30 minutes. Cytokine signaling was stopped by the addition of 50 μl of 4% paraformaldehyde for an additional 30 minutes.

Cells were centrifuged and washed with PBS/1% BSA followed by resuspension in ice cold methanol to permeabilize cells. After 30 minutes, cells were washed twice in PBS/1% BSA before resuspending in 50 μl PBS/1% BSA containing human Fc block (ThermoFisher) at recommended concentration. Cells were incubated on ice for 20 minutes and staining antibody panel was prepared.

Anti-human CD14-BV421, anti-human CD20-PE, anti-human CD3-PE-Cy5, anti-human CD4-eFluor450, and anti-pSTAT3 AF488 were added to cells at recommended concentrations in an additional 50 μl volume. Cells were stained with antibodies for 30 minutes before washing twice with PBS/1% BSA and analyzing on a ZE5 (Biorad) flow cytometer. Data was analyzed using Flowjo software and EC50 potency curves determined using Graphpad Prism. The tested human variants displayed a ˜25-fold range of potencies. EC50 values are reported in Table 4.

Determination of IL-21-Fc variant (human and mouse) signaling in mouse splenocytes (BioIVT) was performed as in human PBMCs with the following modifications. Following thawing, cells were recovered in warm media for 2 hours at 37 C. Cells were labeled with a titration of IL-21-Fc variants ranging from 0.1 pM to 100 nM. The antibody panel used consisted of anti-mouse CD11b-BV421, anti-mouse CD19 PE, anti-mouse CD3-PE-Cy5, anti-mouse CD4-eFluor450, and anti-pSTAT3 AF488. The tested mouse variants displayed a ˜400-fold range of potencies, and some tested human variants demonstrated >1,000-fold potency reduction relative to wild-type mouse IL-21. EC50 values are reported in Table 4.

### Example 4: Identification of Stabilized IL-21 Variants Using AlphaSeq™

Because of the relatively weak binding observed between IL-21 and IL-21R in unbuffered media (see, for examples, FIG. 4A) and the high frequency of IL-21 SSM variants with apparently enhanced affinity to IL-21R (FIG. 5A), it was hypothesized that variants with apparently enhanced affinity represented stabilized or expression-enhancing variants of IL-21. By defining putative stabilized variants as those with a half-log increase in apparent binding affinity to IL-21R relative to the wild-type average, 166 IL-21 single-mutant stabilized variants were identified. These 166 IL-21 single-mutant stabilized variants, predicted affinities, and amino acid sequences (SEQ ID NOs 293-458) are provided in Table 8 below. Such variants are desirable for their potential utility as IL-21-based therapeutics.

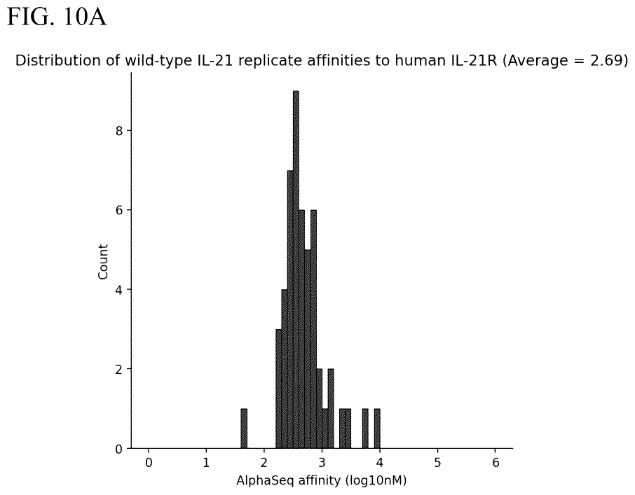

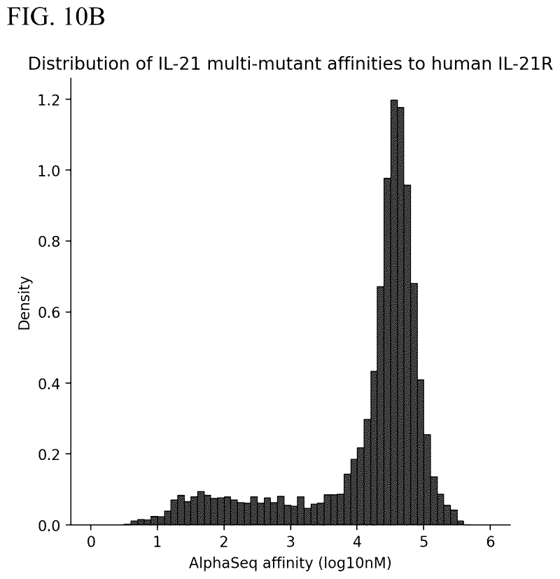

Based on this hypothesis, a second library containing human IL-21 variants with 2, 3, 4, or 5 substitutions from wild-type IL-21 was generated and screened for binding to human IL-21R in unbuffered media. Wild-type copies of IL-21 were included and showed an affinity of 490 nM to IL-21R (log₁₀ nM=2.69 in FIG. 10A). The variant library contained many IL-21 variants with reduced affinity to IL-21R, but also a set with increased affinity (FIG. 10B). By defining stabilized variants as those with a half-log increase in apparent binding affinity to IL-21R relative to the wild-type average, 425 stabilized variants were identified. These 425 stabilized variants, combinations of mutations, predicted affinities, and amino acid sequences (SEQ ID NOs 459-883) are provided in Table 9 below.

To test whether stabilized IL-21 variants identified using AlphaSeq™ also demonstrated improved expression or stability as soluble proteins, a subset of the highest apparent stabilized variants were produced as Fc fusion proteins in EpiCHO™ cells. In addition to producing top hits identified directly from AlphaSeq™, a subset of rational combinations of substitutions were also designed, spanning between 3 and 9 mutations from wild-type IL-21. The rationally designed combinations of substitutions and amino acid sequences are provided in Table 6 below. CHO expression titers and analytical SEC percent main peaks for IL-21 Fc fusions are shown in Table 7. Of the seven tested high-affinity variants identified in AlphaSeq™, three had increased CHO expression titers and purity relative to wild-type IL-21: IL-21_3610, IL-21_4177, and IL-21_0910. All of the five tested rationally designed IL-21 variants showed markedly increased titers, but only one had purity by analytical SEC>80%: IL-21_5mut.

TABLE 6
Rationally Combined IL-21 Variants

| | | | SEQ |
|---|---|---|---|
| Variant | | | ID |
| Name | Mutations | Sequence | NO |
| IL-21_9mut | H35F, | MRSSPGNMERIVICLMVIFL | 288 |
| | N91E, | GTLVHKSSSQGQDREMIRMR | |
| | K101E, | QLIDIVDQLKNYVNDLVPEF | |
| | N111E, | LPAPEDVETNCEWSAFSCFQ | |
| | R114G, | KAQLKSANTGENERIINVSI | |
| | R115G, | EKLKRKPPSTEAGGGQKDTL | |
| | H118D, | ECPSCDSYEKKPPKEFLERF | |
| | R119T, | KSLLQKMIHQHLSSRTHGSE | |
| | T121E | DS | |
| IL-21_3mut | H35F, | MRSSPGNMERIVICLMVIFL | 289 |
| | N91E, | GTLVHKSSSQGQDREMIRMR | |
| | K101E | QLIDIVDQLKNYVNDLVPEF | |
| | | LPAPEDVETNCEWSAFSCFQ | |
| | | KAQLKSANTGENERIINVSI | |
| | | EKLKRKPPSTNAGRRQKHRL | |
| | | TCPSCDSYEKKPPKEFLERF | |
| | | KSLLQKMIHQHLSSRTHGSE | |
| | | DS | |
| IL-21_6mut | N111E, | MRSSPGNMERIVICLMVIFL | 290 |
| | R114G, | GTLVHKSSSQGQDRHMIRMR | |
| | R115G, | QLIDIVDQLKNYVNDLVPEF | |
| | H118D, | LPAPEDVETNCEWSAFSCFQ | |
| | R119T, | KAQLKSANTGNNERIINVSI | |
| | T121E | KKLKRKPPSTEAGGGQKDTL | |
| | | ECPSCDSYEKKPPKEFLERF | |
| | | KSLLQKMIHQHLSSRTHGSE | |
| | | DS | |
| IL-21_4mut | N111E, | MRSSPGNMERIVICLMVIFL | 291 |
| | R115G, | GTLVHKSSSQGQDRHMIRMR | |
| | H118D, | QLIDIVDQLKNYVNDLVPEF | |
| | T121E | LPAPEDVETNCEWSAFSCFQ | |
| | | KA | |
| | | QLKSANTGNNERIINVSIKK | |
| | | LKRKPPSTEAGRGQKDRLEC | |
| | | PSCDSYEKKPPKEFLERFKS | |
| | | LLQKMIHQHLSSRTHGSEDS | |
| IL-21_5mut | H35F, | MRSSPGNMERIVICLMVIFL | 292 |
| | R114G, | GTLVHKSSSQGQDREMIRMR | |
| | R115G, | QLIDIVDQLKNYVNDLVPEF | |
| | H118D, | LPAPEDVETNCEWSAFSCFQ | |
| | R119T, | KAQLKSANTGNNERIINVSI | |
| | T121E | KKLKRKPPSTNAGGGQKDTL | |
| | | ECPSCDSYEKKPPKEFLERF | |
| | | KSLLQKMIHQHLSSRTHGSE | |
| | | DS | |

TABLE 7
Protein Expression Yields and Purity for
Stabilized IL-21-Fc Fusion Proteins

| Variant | Titer (ug/mL) | Percent main peak |
|---|---|---|
| IL-21_WT | 54.0 | 55.5 |
| IL-21_2988 | 68.6 | 48.2 |
| IL-21_3610 | 189.9 | 91.5 |
| IL-21_4177 | 114.0 | 90.2 |
| IL-21_4219 | 97.4 | 57.2 |
| IL-21_0910 | 186.1 | 82.0 |
| IL-21_4818 | 88.3 | 57.4 |
| IL-21_4687 | 47.1 | 15.4 |
| IL-21_9mut | 170.5 | 71.7 |
| IL-21_3mut | 249.4 | 67.8 |
| IL-21_6mut | 139.3 | 62.5 |
| IL-21_4mut | 141.3 | 67.2 |
| IL-21_5mut | 202.8 | 84.9 |

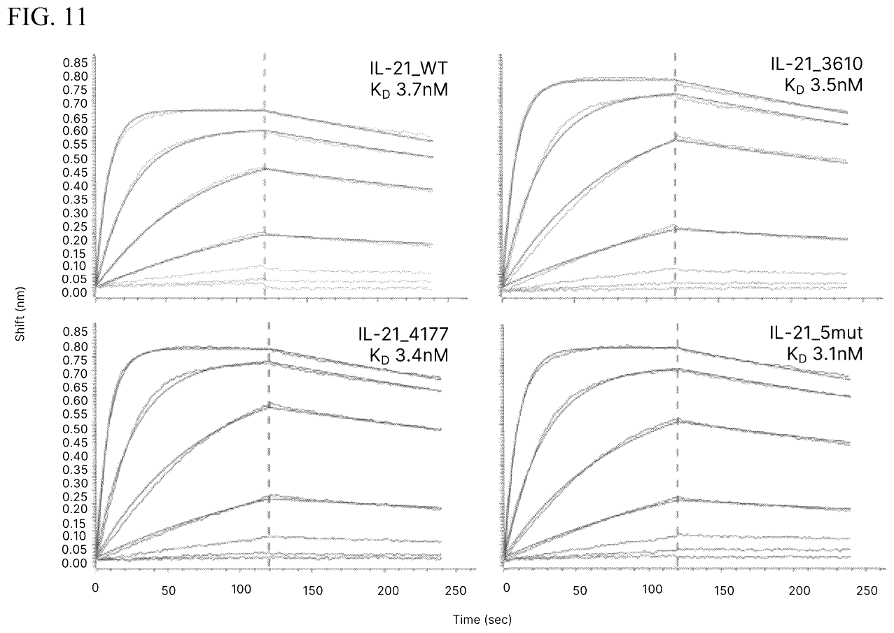

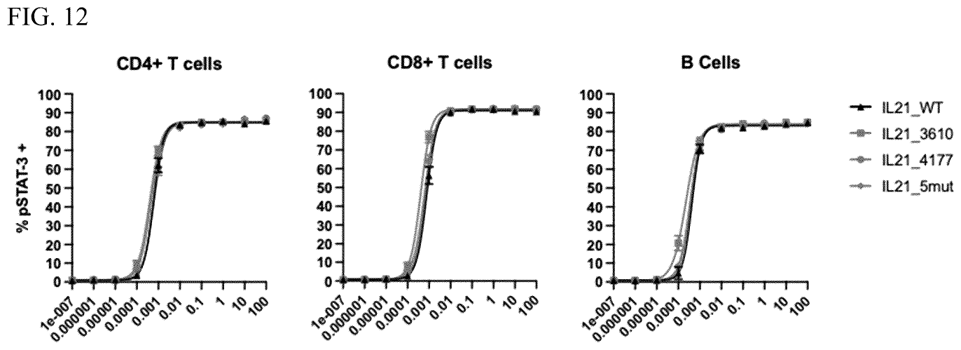

The three highest purity hits from tested IL-21-Fc fusions (IL-21_3610, IL-21_4177, and IL-21_5mut) were tested by BLI for binding affinity to IL-21R to confirm that stabilizing mutations did not affect the ability of IL-21 to bind its receptor (FIG. 11). The IL-21-Fc variants also did not show any change in signaling potency on human PBMCs relative to wild-type IL-21-Fc (FIG. 12).

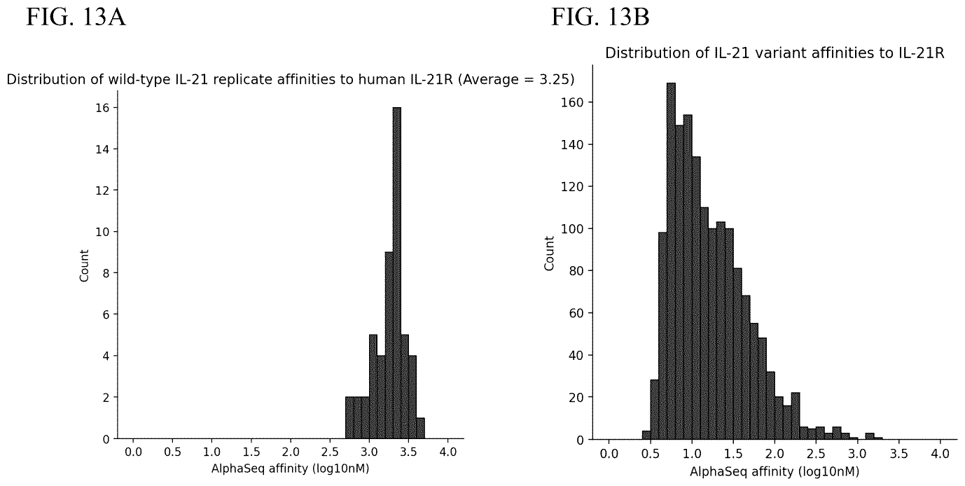

To identify further stabilized variants, a second iteration of AlphaSeq™ was performed using a library of IL-21 variants consisting of combinations of mutations identified as stabilizing in the first library (e.g., one hit from the first library, IL-21_4177, contained substitution H35I and another hit, IL-21_3610, contained substitution P107V, so the combination library tested the combination variant IL-21_H35I_P107V). Wild-type copies of IL-21 were included and showed an affinity of 1778 nM to IL-21R (log₁₀ nM=3.25 in FIG. 13A). The library contained almost all stabilized variants, defining stabilized variants as those with a half-log increase in apparent binding affinity to IL-21R relative to the wild-type average (FIG. 13B). By this definition, 1513 stabilized variants were identified. These 1513 stabilized variants, combinations of mutations, predicted affinities, and amino acid sequences (SEQ ID NOs 884-2396) are provided in Table 10 below.

TABLE 8
IL-21 Single-Mutant Stabilized Variants

| | | | SEQ |
|---|---|---|---|
| Variant | Predicted | | ID |
| Name | Affinity | Amino Acid Sequence | NO |
| IL-21_ | 0.875 | MRSSPGNMERIVICLMVIFLGTLVHKSSSQGQDRHMIRMRQLIDI | 293 |
| V98D | | VDQLKNYVNDLVPEFLPAPEDVETNCEWSAFSCFQKAQLKSANTG | |
| | | NNERIINDSIKKLKRKPPSTNAGRRQKHRLTCPSCDSYEKKPPKE | |
| | | FLERFKSLLQKMIHQHLSSRTHGSEDS | |
| IL-21_ | 0.945 | MRSSPGNMERIVICLMVIFLGTLVHKSSSQGQDRHMIRMRQLIDI | 294 |
| K81D | | VDQLKNYVNDLVPEFLPAPEDVETNCEWSAFSCFQDAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 0.993 | MRSSPGNMERIVICLMVIFLGTLVHKSSSQGQDRHMIRMRQLIDI | 295 |
| K101E | | VDQLKNYVNDLVPEFLPAPEDVETNCEWSAFSCFQKAQLKSANTG | |
| | | NNERIINVSIEKLKRKPPSTNAGRRQKHRLTCPSCDSYEKKPPKE | |
| | | FLERFKSLLQKMIHQHLSSRTHGSEDS | |
| IL-21_ | 1.019 | MRSSPGNMERIVICLMVIFLGTLVHKSSSQGQDRHMIRMRQLIDI | 296 |
| R115E | | VDQLKNYVNDLVPEFLPAPEDVETNCEWSAFSCFQKAQLKSANTG | |
| | | NNERIINVSIKKLKRKPPSTNAGREQKHRLTCPSCDSYEKKPPKE | |
| | | FLERFKSLLQKMIHQHLSSRTHGSEDS | |
| IL-21_ | 1.059 | MRSSPGNMERIVICLMVIFLGTLVHKSSSQGQDRHMIRMRQLIDI | 297 |
| K81E | | VDQLKNYVNDLVPEFLPAPEDVETNCEWSAFSCFQEAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.098 | MRSSPGNMERIVICLMVIFLGTLVHKSSSQGQDRHMIRMRQLIDI | 298 |
| Q83E | | VDQLKNYVNDLVPEFLPAPEDVETNCEWSAFSCFQKAELKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.174 | MRSSPGNMERIVICLMVIFLGTLVHKSSSQGQDRHMIRMRQLIDI | 299 |
| R94E | | VDQLKNYVNDLVPEFLPAPEDVETNCEWSAFSCFQKAQLKSANTG | |
| | | NNEEIINVSIKKLKRKPPSTNAGRRQKHRLTCPSCDSYEKKPPKE | |
| | | FLERFKSLLQKMIHQHLSSRTHGSEDS | |
| IL-21_ | 1.219 | MRSSPGNMERIVICLMVIFLGTLVHKSSSQGQDRHMIRMRQLIDI | 300 |
| K101A | | VDQLKNYVNDLVPEFLPAPEDVETNCEWSAFSCFQKAQLKSANTG | |
| | | NNERIINVSIAKLKRKPPSTNAGRRQKHRLTCPSCDSYEKKPPKE | |
| | | FLERFKSLLQKMIHQHLSSRTHGSEDS | |
| IL-21_ | 1.238 | MRSSPGNMERIVICLMVIFLGTLVHKSSSQGQDRHMIRMRQLIDI | 301 |
| K101D | | VDQLKNYVNDLVPEFLPAPEDVETNCEWSAFSCFQKAQLKSANTG | |
| | | NNERIINVSIDKLKRKPPSTNAGRRQKHRLTCPSCDSYEKKPPKE | |
| | | FLERFKSLLQKMIHQHLSSRTHGSEDS | |
| IL-21_ | 1.281 | MRSSPGNMERIVICLMVIFLGTLVHKSSSQGQDRHMIRMRQLIDI | 302 |
| R94D | | VDQLKNYVNDLVPEFLPAPEDVETNCEWSAFSCFQKAQLKSANTG | |
| | | NNEDIINVSIKKLKRKPPSTNAGRRQKHRLTCPSCDSYEKKPPKE | |
| | | FLERFKSLLQKMIHQHLSSRTHGSEDS | |
| IL-21_ | 1.283 | MRSSPGNMERIVICLMVIFLGTLVHKSSSQGQDRDMIRMRQLIDI | 303 |
| H35D | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.296 | MRSSPGNMERIVICLMVIFLGTLVHKSSSQGQDRHMIRMRQLIDI | 304 |
| K102Q | | VDQLKNYVNDLVPEFLPAPEDVETNCEWSAFSCFQKAQLKSANTG | |
| | | NNERIINVSIKQLKRKPPSTNAGRRQKHRLTCPSCDSYEKKPPKE | |
| | | FLERFKSLLQKMIHQHLSSRTHGSEDS | |
| IL-21_ | 1.299 | MRSSPGNMERIVICLMVIFLGTLVHKSSSQGQDRHMIRMRQLIDI | 305 |
| K81N | | VDQLKNYVNDLVPEFLPAPEDVETNCEWSAFSCFQNAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.304 | MRSSPGNMERIVICLMVIFLGTLVHKSSSQGQDREMIRMRQLIDI | 306 |
| H35E | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.335 | MRSSPGNMERIVICLMVIFLGTLVHKSSSQGQDRHMIRMRQLIDI | 307 |
| V98E | | VDQLKNYVNDLVPEFLPAPEDVETNCEWSAFSCFQKAQLKSANTG | |
| | | NNERIINESIKKLKRKPPSTNAGRRQKHRLTCPSCDSYEKKPPKE | |
| | | FLERFKSLLQKMIHQHLSSRTHGSEDS | |
| IL-21_ | 1.362 | MRSSPGNMERIVICLMVIFLGTLVHKSSSQGQDRHMIRMRQLIDI | 308 |
| R119E | | VDQLKNYVNDLVPEFLPAPEDVETNCEWSAFSCFQKAQLKSANTG | |
| | | NNERIINVSIKKLKRKPPSTNAGRRQKHELTCPSCDSYEKKPPKE | |
| | | FLERFKSLLQKMIHQHLSSRTHGSEDS | |
| IL-21_ | 1.368 | MRSSPGNMERIVICLMVIFLGTLVHKSSSQGQDRHMIRMRQLIDI | 309 |
| R114D | | VDQLKNYVNDLVPEFLPAPEDVETNCEWSAFSCFQKAQLKSANTG | |
| | | NNERIINVSIKKLKRKPPSTNAGDRQKHRLTCPSCDSYEKKPPKE | |
| | | FLERFKSLLQKMIHQHLSSRTHGSEDS | |
| IL-21_ | 1.443 | MRSSPGNMERIVICLMVIFLGTLVHKSSSQGQDRHMIRMRQLIDI | 310 |
| K81S | | VDQLKNYVNDLVPEFLPAPEDVETNCEWSAFSCFQSAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.45 | MRSSPGNMERIVICLMVIFLGTLVHKSSSQGQDRHMIRMRQLIDI | 311 |
| K117E | | VDQLKNYVNDLVPEFLPAPEDVETNCEWSAFSCFQKAQLKSANTG | |
| | | NNERIINVSIKKLKRKPPSTNAGRRQEHRLTCPSCDSYEKKPPKE | |
| | | FLERFKSLLQKMIHQHLSSRTHGSEDS | |
| IL-21_ | 1.503 | MRSSPGNMERIVICLMVIFLGTLVHKSSSQGQDRHMIRMRQLIDI | 312 |
| R114P | | VDQLKNYVNDLVPEFLPAPEDVETNCEWSAFSCFQKAQLKSANTG | |
| | | NNERIINVSIKKLKRKPPSTNAGPRQKHRLTCPSCDSYEKKPPKE | |
| | | FLERFKSLLQKMIHQHLSSRTHGSEDS | |
| IL-21_ | 1.51 | MRSSPGNMERIVICLMVIFLGTLVHKSSSQGQDRHMIRMRQLIDI | 313 |
| K106I | | VDQLKNYVNDLVPEFLPAPEDVETNCEWSAFSCFQKAQLKSANTG | |
| | | NNERIINVSIKKLKRIPPSTNAGRRQKHRLTCPSCDSYEKKPPKE | |
| | | FLERFKSLLQKMIHQHLSSRTHGSEDS | |
| IL-21_ | 1.52 | MRSSPGNMERIVICLMVIFLGTLVHKSSSQGQDRHMIRMRQLIDI | 314 |
| R114Q | | VDQLKNYVNDLVPEFLPAPEDVETNCEWSAFSCFQKAQLKSANTG | |
| | | NNERIINVSIKKLKRKPPSTNAGQRQKHRLTCPSCDSYEKKPPKE | |
| | | FLERFKSLLQKMIHQHLSSRTHGSEDS | |
| IL-21_ | 1.537 | MRSSPGNMERIVICLMVIFLGTLVHKSSSQGQDRHMIRMRQLIDI | 315 |
| K81M | | VDQLKNYVNDLVPEFLPAPEDVETNCEWSAFSCFQMAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.552 | MRSSPGNMERIVICLMVIFLGTLVHKSSSQGQDRHMIRMRQLIDI | 316 |
| R114L | | VDQLKNYVNDLVPEFLPAPEDVETNCEWSAFSCFQKAQLKSANTG | |
| | | NNERIINVSIKKLKRKPPSTNAGLRQKHRLTCPSCDSYEKKPPKE | |
| | | FLERFKSLLQKMIHQHLSSRTHGSEDS | |
| IL-21_ | 1.565 | MRSSPGNMERIVICLMVIFLGTLVHKSSSQGQDRHMIRMRQLIDI | 317 |
| R119D | | VDQLKNYVNDLVPEFLPAPEDVETNCEWSAFSCFQKAQLKSANTG | |
| | | NNERIINVSIKKLKRKPPSTNAGRRQKHDLTCPSCDSYEKKPPKE | |
| | | FLERFKSLLQKMIHQHLSSRTHGSEDS | |
| IL-21_ | 1.565 | MRSSPGNMERIVICLMVIFLGTLVHKSSSQGQDRHMIRMRQLIDI | 318 |
| R94A | | VDQLKNYVNDLVPEFLPAPEDVETNCEWSAFSCFQKAQLKSANTG | |
| | | NNEAIINVSIKKLKRKPPSTNAGRRQKHRLTCPSCDSYEKKPPKE | |
| | | FLERFKSLLQKMIHQHLSSRTHGSEDS | |
| IL-21_ | 1.592 | MRSSPGNMERIVICLMVIFLGTLVHKSSSQGQDRHMIRMRQLIDI | 319 |
| K81T | | VDQLKNYVNDLVPEFLPAPEDVETNCEWSAFSCFQTAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.595 | MRSSPGNMERIVICLMVIFLGTLVHKSSSQGQDRHMIRMRQLIDI | 320 |
| H118D | | VDQLKNYVNDLVPEFLPAPEDVETNCEWSAFSCFQKAQLKSANTG | |
| | | NNERIINVSIKKLKRKPPSTNAGRRQKDRLTCPSCDSYEKKPPKE | |
| | | FLERFKSLLQKMIHQHLSSRTHGSEDS | |
| IL-21_ | 1.596 | MRSSPGNMERIVICLMVIFLGTLVHKSSSQGQDRHMIRMRQLIDI | 321 |
| K81G | | VDQLKNYVNDLVPEFLPAPEDVETNCEWSAFSCFQGAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.598 | MRSSPGNMERIVICLMVIFLGTLVHKSSSQGQDRHMIRMRQLIDI | 322 |
| K104L | | VDQLKNYVNDLVPEFLPAPEDVETNCEWSAFSCFQKAQLKSANTG | |
| | | NNERIINVSIKKLLRKPPSTNAGRRQKHRLTCPSCDSYEKKPPKE | |
| | | FLERFKSLLQKMIHQHLSSRTHGSEDS | |
| IL-21_ | 1.607 | MRSSPGNMERIVICLMVIFLGTLVHKSSSQGQDRYMIRMRQLIDI | 323 |
| H35Y | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.61 | MRSSPGNMERIVICLMVIFLGTLVHKSSSQGQDRWMIRMRQLIDI | 324 |
| H35W | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.631 | MRSSPGNMERIVICLMVIFLGTLVHKSSSQGQDRHMIRMRQLIDI | 325 |
| R115D | | VDQLKNYVNDLVPEFLPAPEDVETNCEWSAFSCFQKAQLKSANTG | |
| | | NNERIINVSIKKLKRKPPSTNAGRDQKHRLTCPSCDSYEKKPPKE | |
| | | FLERFKSLLQKMIHQHLSSRTHGSEDS | |
| IL-21_ | 1.639 | MRSSPGNMERIVICLMVIFLGTLVHKSSSQGQDRHMIRMRQLIDI | 326 |
| H118E | | VDQLKNYVNDLVPEFLPAPEDVETNCEWSAFSCFQKAQLKSANTG | |
| | | NNERIINVSIKKLKRKPPSTNAGRRQKERLTCPSCDSYEKKPPKE | |
| | | FLERFKSLLQKMIHQHLSSRTHGSEDS | |
| IL-21_ | 1.66 | MRSSPGNMERIVICLMVIFLGTLVHKSSSQGQDRHMIRMRQLIDI | 327 |
| K81A | | VDQLKNYVNDLVPEFLPAPEDVETNCEWSAFSCFQAAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.667 | MRSSPGNMERIVICLMVIFLGTLVHKSSSQGQDRHMIRMRQLIDI | 328 |
| S77E | | VDQLKNYVNDLVPEFLPAPEDVETNCEWSAFECFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.686 | MRSSPGNMERIVICLMVIFLGTLVHKSSSQGQDRHMIRMRQLIDI | 329 |
| K106Y | | VDQLKNYVNDLVPEFLPAPEDVETNCEWSAFSCFQKAQLKSANTG | |
| | | NNERIINVSIKKLKRYPPSTNAGRRQKHRLTCPSCDSYEKKPPKE | |
| | | FLERFKSLLQKMIHQHLSSRTHGSEDS | |
| IL-21_ | 1.686 | MRSSPGNMERIVICLMVIFLGTLVHKSSSQGQDRHMIRMRQLIDI | 330 |
| K117D | | VDQLKNYVNDLVPEFLPAPEDVETNCEWSAFSCFQKAQLKSANTG | |
| | | NNERIINVSIKKLKRKPPSTNAGRRQDHRLTCPSCDSYEKKPPKE | |
| | | FLERFKSLLQKMIHQHLSSRTHGSEDS | |
| IL-21_ | 1.688 | MRSSPGNMERIVICLMVIFLGTLVHKSSSQGQDRHMIRMRQLIDI | 331 |
| K101S | | VDQLKNYVNDLVPEFLPAPEDVETNCEWSAFSCFQKAQLKSANTG | |
| | | NNERIINVSISKLKRKPPSTNAGRRQKHRLTCPSCDSYEKKPPKE | |
| | | FLERFKSLLQKMIHQHLSSRTHGSEDS | |
| IL-21_ | 1.709 | MRSSPGNMERIVICLMVIFLGTLVHKSSSQGQDRHMIRMRQLIDI | 332 |
| K102G | | VDQLKNYVNDLVPEFLPAPEDVETNCEWSAFSCFQKAQLKSANTG | |
| | | NNERIINVSIKGLKRKPPSTNAGRRQKHRLTCPSCDSYEKKPPKE | |
| | | FLERFKSLLQKMIHQHLSSRTHGSEDS | |
| IL-21_ | 1.716 | MRSSPGNMERIVICLMVIFLGTLVHKSSSQGQDRHMIRMRQLIDI | 333 |
| R114I | | VDQLKNYVNDLVPEFLPAPEDVETNCEWSAFSCFQKAQLKSANTG | |
| | | NNERIINVSIKKLKRKPPSTNAGIRQKHRLTCPSCDSYEKKPPKE | |
| | | FLERFKSLLQKMIHQHLSSRTHGSEDS | |
| IL-21_ | 1.723 | MRSSPGNMERIVICLMVIFLGTLVHKSSSQGQDRHMIRMRQLIDI | 334 |
| K102E | | VDQLKNYVNDLVPEFLPAPEDVETNCEWSAFSCFQKAQLKSANTG | |
| | | NNERIINVSIKELKRKPPSTNAGRRQKHRLTCPSCDSYEKKPPKE | |
| | | FLERFKSLLQKMIHQHLSSRTHGSEDS | |
| IL-21_ | 1.735 | MRSSPGNMERIVICLMVIFLGTLVHKSSSQGQDRHMIRMRQLIDI | 335 |
| K106V | | VDQLKNYVNDLVPEFLPAPEDVETNCEWSAFSCFQKAQLKSANTG | |
| | | NNERIINVSIKKLKRVPPSTNAGRRQKHRLTCPSCDSYEKKPPKE | |
| | | FLERFKSLLQKMIHQHLSSRTHGSEDS | |
| IL-21_ | 1.741 | MRSSPGNMERIVICLMVIFLGTLVHKSSSQGQDRHMIRMRQLIDI | 336 |
| K102A | | VDQLKNYVNDLVPEFLPAPEDVETNCEWSAFSCFQKAQLKSANTG | |
| | | NNERIINVSIKALKRKPPSTNAGRRQKHRLTCPSCDSYEKKPPKE | |
| | | FLERFKSLLQKMIHQHLSSRTHGSEDS | |
| IL-21_ | 1.742 | MRSSPGNMERIVICLMVIFLGTLVHKSSSQGQDRHMIRMRQLIDI | 337 |
| R115P | | VDQLKNYVNDLVPEFLPAPEDVETNCEWSAFSCFQKAQLKSANTG | |
| | | NNERIINVSIKKLKRKPPSTNAGRPQKHRLTCPSCDSYEKKPPKE | |
| | | FLERFKSLLQKMIHQHLSSRTHGSEDS | |
| IL-21_ | 1.747 | MRSSPGNMERIVICLMVIFLGTLVHKSSSQGQDRHMIRMRQLIDI | 338 |
| T110D | | VDQLKNYVNDLVPEFLPAPEDVETNCEWSAFSCFQKAQLKSANTG | |
| | | NNERIINVSIKKLKRKPPSDNAGRRQKHRLTCPSCDSYEKKPPKE | |
| | | FLERFKSLLQKMIHQHLSSRTHGSEDS | |
| IL-21_ | 1.76 | MRSSPGNMERIVICLMVIFLGTLVHKSSSQGQDRHMIRMRQLIDI | 339 |
| R115N | | VDQLKNYVNDLVPEFLPAPEDVETNCEWSAFSCFQKAQLKSANTG | |
| | | NNERIINVSIKKLKRKPPSTNAGRNQKHRLTCPSCDSYEKKPPKE | |
| | | FLERFKSLLQKMIHQHLSSRTHGSEDS | |
| IL-21_ | 1.769 | MRSSPGNMERIVICLMVIFLGTLVHKSSSQGQDRFMIRMRQLIDI | 340 |
| H35F | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.774 | MRSSPGNMERIVICLMVIFLGTLVHKSSSQGQDRHMIRMRQLIDI | 341 |
| K81Q | | VDQLKNYVNDLVPEFLPAPEDVETNCEWSAFSCFQQAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.778 | MRSSPGNMERIVICLMVIFLGTLVHKSSSQGQDRHMIRMRQLIDI | 342 |
| R119T | | VDQLKNYVNDLVPEFLPAPEDVETNCEWSAFSCFQKAQLKSANTG | |
| | | NNERIINVSIKKLKRKPPSTNAGRRQKHTLTCPSCDSYEKKPPKE | |
| | | FLERFKSLLQKMIHQHLSSRTHGSEDS | |
| IL-21_ | 1.785 | MRSSPGNMERIVICLMVIFLGTLVHKSSSQGQDRHMIRMRQLIDI | 343 |
| R114M | | VDQLKNYVNDLVPEFLPAPEDVETNCEWSAFSCFQKAQLKSANTG | |
| | | NNERIINVSIKKLKRKPPSTNAGMRQKHRLTCPSCDSYEKKPPKE | |
| | | FLERFKSLLQKMIHQHLSSRTHGSEDS | |
| IL-21_ | 1.789 | MRSSPGNMERIVICLMVIFLGTLVHKSSSQGQDRHMIRMRQLIDI | 344 |
| N97E | | VDQLKNYVNDLVPEFLPAPEDVETNCEWSAFSCFQKAQLKSANTG | |
| | | NNERIIEVSIKKLKRKPPSTNAGRRQKHRLTCPSCDSYEKKPPKE | |
| | | FLERFKSLLQKMIHQHLSSRTHGSEDS | |
| IL-21_ | 1.814 | MRSSPGNMERIVICLMVIFLGTLVHKSSSQGQDRHMIRMRQLIDI | 345 |
| R94Q | | VDQLKNYVNDLVPEFLPAPEDVETNCEWSAFSCFQKAQLKSANTG | |
| | | NNEQIINVSIKKLKRKPPSTNAGRRQKHRLTCPSCDSYEKKPPKE | |
| | | FLERFKSLLQKMIHQHLSSRTHGSEDS | |
| IL-21_ | 1.818 | MRSSPGNMERIVICLMVIFLGTLVHKSSSQGQDRHMIRMRQLIDI | 346 |
| K117N | | VDQLKNYVNDLVPEFLPAPEDVETNCEWSAFSCFQKAQLKSANTG | |
| | | NNERIINVSIKKLKRKPPSTNAGRRQNHRLTCPSCDSYEKKPPKE | |
| | | FLERFKSLLQKMIHQHLSSRTHGSEDS | |
| IL-21_ | 1.819 | MRSSPGNMERIVICLMVIFLGTLVHKSSSQGQDRHMIRMRQLIDI | 347 |
| R94N | | VDQLKNYVNDLVPEFLPAPEDVETNCEWSAFSCFQKAQLKSANTG | |
| | | NNENIINVSIKKLKRKPPSTNAGRRQKHRLTCPSCDSYEKKPPKE | |
| | | FLERFKSLLQKMIHQHLSSRTHGSEDS | |
| IL-21_ | 1.82 | MRSSPGNMERIVICLMVIFLGTLVHKSSSQGQDRHMIRMRQLIDI | 348 |
| R119G | | VDQLKNYVNDLVPEFLPAPEDVETNCEWSAFSCFQKAQLKSANTG | |
| | | NNERIINVSIKKLKRKPPSTNAGRRQKHGLTCPSCDSYEKKPPKE | |
| | | FLERFKSLLQKMIHQHLSSRTHGSEDS | |
| IL-21_ | 1.822 | MRSSPGNMERIVICLMVIFLGTLVHKSSSQGQDRHMIRMRQLIDI | 349 |
| R114A | | VDQLKNYVNDLVPEFLPAPEDVETNCEWSAFSCFQKAQLKSANTG | |
| | | NNERIINVSIKKLKRKPPSTNAGARQKHRLTCPSCDSYEKKPPKE | |
| | | FLERFKSLLQKMIHQHLSSRTHGSEDS | |
| IL-21_ | 1.825 | MRSSPGNMERIVICLMVIFLGTLVHKSSSQGQDRHMIRMRQLIDI | 350 |
| R114N | | VDQLKNYVNDLVPEFLPAPEDVETNCEWSAFSCFQKAQLKSANTG | |
| | | NNERIINVSIKKLKRKPPSTNAGNRQKHRLTCPSCDSYEKKPPKE | |
| | | FLERFKSLLQKMIHQHLSSRTHGSEDS | |
| IL-21_ | 1.834 | MRSSPGNMERIVICLMVIFLGTLVHKSSSQGQDRHMIRMRQLIDI | 351 |
| K106F | | VDQLKNYVNDLVPEFLPAPEDVETNCEWSAFSCFQKAQLKSANTG | |
| | | NNERIINVSIKKLKRFPPSTNAGRRQKHRLTCPSCDSYEKKPPKE | |
| | | FLERFKSLLQKMIHQHLSSRTHGSEDS | |
| IL-21_ | 1.851 | MRSSPGNMERIVICLMVIFLGTLVHKSSSQGQDRHMIRMRQLIDI | 352 |
| R114G | | VDQLKNYVNDLVPEFLPAPEDVETNCEWSAFSCFQKAQLKSANTG | |
| | | NNERIINVSIKKLKRKPPSTNAGGRQKHRLTCPSCDSYEKKPPKE | |
| | | FLERFKSLLQKMIHQHLSSRTHGSEDS | |
| IL-21_ | 1.862 | MRSSPGNMERIVICLMVIFLGTLVHKSSSQGQDRHMIRMRQLIDI | 353 |
| K101Q | | VDQLKNYVNDLVPEFLPAPEDVETNCEWSAFSCFQKAQLKSANTG | |
| | | NNERIINVSIQKLKRKPPSTNAGRRQKHRLTCPSCDSYEKKPPKE | |
| | | FLERFKSLLQKMIHQHLSSRTHGSEDS | |
| IL-21_ | 1.867 | MRSSPGNMERIVICLMVIFLGTLVHKSSSQGQDRHMIRMRQLIDI | 354 |
| R115Q | | VDQLKNYVNDLVPEFLPAPEDVETNCEWSAFSCFQKAQLKSANTG | |
| | | NNERIINVSIKKLKRKPPSTNAGRQQKHRLTCPSCDSYEKKPPKE | |
| | | FLERFKSLLQKMIHQHLSSRTHGSEDS | |
| IL-21_ | 1.87 | MRSSPGNMERIVICLMVIFLGTLVHKSSSQGQDRHMIRMRQLIDI | 355 |
| R119P | | VDQLKNYVNDLVPEFLPAPEDVETNCEWSAFSCFQKAQLKSANTG | |
| | | NNERIINVSIKKLKRKPPSTNAGRRQKHPLTCPSCDSYEKKPPKE | |
| | | FLERFKSLLQKMIHQHLSSRTHGSEDS | |
| IL-21_ | 1.871 | MRSSPGNMERIVICLMVIFLGTLVHKSSSQGQDRHMIRMRQLIDI | 356 |
| R115L | | VDQLKNYVNDLVPEFLPAPEDVETNCEWSAFSCFQKAQLKSANTG | |
| | | NNERIINVSIKKLKRKPPSTNAGRLQKHRLTCPSCDSYEKKPPKE | |
| | | FLERFKSLLQKMIHQHLSSRTHGSEDS | |
| IL-21_ | 1.872 | MRSSPGNMERIVICLMVIFLGTLVHKSSSQGQDRHMIRMRQLIDI | 357 |
| N111E | | VDQLKNYVNDLVPEFLPAPEDVETNCEWSAFSCFQKAQLKSANTG | |
| | | NNERIINVSIKKLKRKPPSTEAGRRQKHRLTCPSCDSYEKKPPKE | |
| | | FLERFKSLLQKMIHQHLSSRTHGSEDS | |
| IL-21_ | 1.902 | MRSSPGNMERIVICLMVIFLGTLVHKSSSQGQDRTMIRMRQLIDI | 358 |
| H35T | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.903 | MRSSPGNMERIVICLMVIFLGTLVHKSSSQGQDRHMIRMRQLIDI | 359 |
| K104S | | VDQLKNYVNDLVPEFLPAPEDVETNCEWSAFSCFQKAQLKSANTG | |
| | | NNERIINVSIKKLSRKPPSTNAGRRQKHRLTCPSCDSYEKKPPKE | |
| | | FLERFKSLLQKMIHQHLSSRTHGSEDS | |
| IL-21_ | 1.916 | MRSSPGNMERIVICLMVIFLGTLVHKSSSQGQDRHMIRMRQLIDI | 360 |
| R115F | | VDQLKNYVNDLVPEFLPAPEDVETNCEWSAFSCFQKAQLKSANTG | |
| | | NNERIINVSIKKLKRKPPSTNAGRFQKHRLTCPSCDSYEKKPPKE | |
| | | FLERFKSLLQKMIHQHLSSRTHGSEDS | |
| IL-21_ | 1.936 | MRSSPGNMERIVICLMVIFLGTLVHKSSSQGQDRHMIRMRQLIDI | 361 |
| R115W | | VDQLKNYVNDLVPEFLPAPEDVETNCEWSAFSCFQKAQLKSANTG | |
| | | NNERIINVSIKKLKRKPPSTNAGRWQKHRLTCPSCDSYEKKPPKE | |
| | | FLERFKSLLQKMIHQHLSSRTHGSEDS | |
| IL-21_ | 1.944 | MRSSPGNMERIVICLMVIFLGTLVHKSSSQGQDRHMIRMRQLIDI | 362 |
| H118T | | VDQLKNYVNDLVPEFLPAPEDVETNCEWSAFSCFQKAQLKSANTG | |
| | | NNERIINVSIKKLKRKPPSTNAGRRQKTRLTCPSCDSYEKKPPKE | |
| | | FLERFKSLLQKMIHQHLSSRTHGSEDS | |
| IL-21_ | 1.965 | MRSSPGNMERIVICLMVIFLGTLVHKSSSQGQDRHMIRMRQLIDI | 363 |
| S77D | | VDQLKNYVNDLVPEFLPAPEDVETNCEWSAFDCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.965 | MRSSPGNMERIVICLMVIFLGTLVHKSSSQGQDRHMIRMRQLIDI | 364 |
| G113D | | VDQLKNYVNDLVPEFLPAPEDVETNCEWSAFSCFQKAQLKSANTG | |
| | | NNERIINVSIKKLKRKPPSTNADRRQKHRLTCPSCDSYEKKPPKE | |
| | | FLERFKSLLQKMIHQHLSSRTHGSEDS | |
| IL-21_ | 1.965 | MRSSPGNMERIVICLMVIFLGTLVHKSSSQGQDRHMIRMRQLIDI | 365 |
| K117G | | VDQLKNYVNDLVPEFLPAPEDVETNCEWSAFSCFQKAQLKSANTG | |
| | | NNERIINVSIKKLKRKPPSTNAGRRQGHRLTCPSCDSYEKKPPKE | |
| | | FLERFKSLLQKMIHQHLSSRTHGSEDS | |
| IL-21_ | 1.969 | MRSSPGNMERIVICLMVIFLGTLVHKSSSQGQDRHMIRMRQLIDI | 366 |
| K102D | | VDQLKNYVNDLVPEFLPAPEDVETNCEWSAFSCFQKAQLKSANTG | |
| | | NNERIINVSIKDLKRKPPSTNAGRRQKHRLTCPSCDSYEKKPPKE | |
| | | FLERFKSLLQKMIHQHLSSRTHGSEDS | |
| IL-21_ | 1.972 | MRSSPGNMERIVICLMVIFLGTLVHKSSSQGQDRHMIRMRQLIDI | 367 |
| R114E | | VDQLKNYVNDLVPEFLPAPEDVETNCEWSAFSCFQKAQLKSANTG | |
| | | NNERIINVSIKKLKRKPPSTNAGERQKHRLTCPSCDSYEKKPPKE | |
| | | FLERFKSLLQKMIHQHLSSRTHGSEDS | |
| IL-21_ | 1.976 | MRSSPGNMERIVICLMVIFLGTLVHKSSSQGQDRHMIRMRQLIDI | 368 |
| K104M | | VDQLKNYVNDLVPEFLPAPEDVETNCEWSAFSCFQKAQLKSANTG | |
| | | NNERIINVSIKKLMRKPPSTNAGRRQKHRLTCPSCDSYEKKPPKE | |
| | | FLERFKSLLQKMIHQHLSSRTHGSEDS | |
| IL-21_ | 1.977 | MRSSPGNMERIVICLMVIFLGTLVHKSSSQGQDRHMIRMRQLIDI | 369 |
| N70D | | VDQLKNYVNDLVPEFLPAPEDVETDCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.978 | MRSSPGNMERIVICLMVIFLGTLVHKSSSQGQDRHMIRMRQLIDI | 370 |
| R119L | | VDQLKNYVNDLVPEFLPAPEDVETNCEWSAFSCFQKAQLKSANTG | |
| | | NNERIINVSIKKLKRKPPSTNAGRRQKHLLTCPSCDSYEKKPPKE | |
| | | FLERFKSLLQKMIHQHLSSRTHGSEDS | |
| IL-21_ | 1.98 | MRSSPGNMERIVICLMVIFLGTLVHKSSSQGQDRHMIRMRQLIDI | 371 |
| A112D | | VDQLKNYVNDLVPEFLPAPEDVETNCEWSAFSCFQKAQLKSANTG | |
| | | NNERIINVSIKKLKRKPPSTNDGRRQKHRLTCPSCDSYEKKPPKE | |
| | | FLERFKSLLQKMIHQHLSSRTHGSEDS | |
| IL-21_ | 1.981 | MRSSPGNMERIVICLMVIFLGTLVHKSSSQGQDRHMIRMRQLIDI | 372 |
| Q48E | | VDELKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.984 | MRSSPGNMERIVICLMVIFLGTLVHKSSSQGQDRHMIRMRQLIDI | 373 |
| R115V | | VDQLKNYVNDLVPEFLPAPEDVETNCEWSAFSCFQKAQLKSANTG | |
| | | NNERIINVSIKKLKRKPPSTNAGRVQKHRLTCPSCDSYEKKPPKE | |
| | | FLERFKSLLQKMIHQHLSSRTHGSEDS | |
| IL-21_ | 1.984 | MRSSPGNMERIVICLMVIFLGTLVHKSSSQGQDRHMIHMRQLIDI | 374 |
| R38H | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.985 | MRSSPGNMERIVICLMVIFLGTLVHKSSSQGQDRHMIRMRELIDI | 375 |
| Q41E | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.992 | MRSSPGNMERIVICLMVIFLGTLVHKSSSQGQDRHMIRMRQLIDI | 376 |
| V57D | | VDQLKNYVNDLDPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 1.998 | MRSSPGNMERIVICLMVIFLGTLVHKSSSQGQDRHMIRMRQLIDI | 377 |
| L61F | | VDQLKNYVNDLVPEFFPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.009 | MRSSPGNMERIVICLMVIFLGTLVHKSSSQGQDRHMIRMRQLIDI | 378 |
| K101N | | VDQLKNYVNDLVPEFLPAPEDVETNCEWSAFSCFQKAQLKSANTG | |
| | | NNERIINVSINKLKRKPPSTNAGRRQKHRLTCPSCDSYEKKPPKE | |
| | | FLERFKSLLQKMIHQHLSSRTHGSEDS | |
| IL-21_ | 2.01 | MRSSPGNMERIVICLMVIFLGTLVHKSSSQGQDRHMIRMRQLIDI | 379 |
| K117Q | | VDQLKNYVNDLVPEFLPAPEDVETNCEWSAFSCFQKAQLKSANTG | |
| | | NNERIINVSIKKLKRKPPSTNAGRRQQHRLTCPSCDSYEKKPPKE | |
| | | FLERFKSLLQKMIHQHLSSRTHGSEDS | |
| IL-21_ | 2.012 | MRSSPGNMERIVICLMVIFLGTLVHKSSSQGQDRVMIRMRQLIDI | 380 |
| H35V | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.013 | MRSSPGNMERIVICLMVIFLGTLVHKSSSQGQDRAMIRMRQLIDI | 381 |
| H35A | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.018 | MRSSPGNMERIVICLMVIFLGTLVHKSSSQGQDRQMIRMRQLIDI | 382 |
| H35Q | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.022 | MRSSPGNMERIVICLMVIFLGTLVHKSSSQGQDRHMIRMRQLIDI | 383 |
| K106L | | VDQLKNYVNDLVPEFLPAPEDVETNCEWSAFSCFQKAQLKSANTG | |
| | | NNERIINVSIKKLKRLPPSTNAGRRQKHRLTCPSCDSYEKKPPKE | |
| | | FLERFKSLLQKMIHQHLSSRTHGSEDS | |
| IL-21_ | 2.022 | MRSSPGNMERIVICLMVIFLGTLVHKSSSQGQDRHMIRMRQLIDI | 384 |
| K101L | | VDQLKNYVNDLVPEFLPAPEDVETNCEWSAFSCFQKAQLKSANTG | |
| | | NNERIINVSILKLKRKPPSTNAGRRQKHRLTCPSCDSYEKKPPKE | |
| | | FLERFKSLLQKMIHQHLSSRTHGSEDS | |
| IL-21_ | 2.03 | MRSSPGNMERIVICLMVIFLGTLVHKSSSQGQDRHMIRMRQLIDI | 385 |
| K102N | | VDQLKNYVNDLVPEFLPAPEDVETNCEWSAFSCFQKAQLKSANTG | |
| | | NNERIINVSIKNLKRKPPSTNAGRRQKHRLTCPSCDSYEKKPPKE | |
| | | FLERFKSLLQKMIHQHLSSRTHGSEDS | |
| IL-21_ | 2.039 | MRSSPGNMERIVICLMVIFLGTLVHKSSSQGQDRHMIRMRQLIDI | 386 |
| R114W | | VDQLKNYVNDLVPEFLPAPEDVETNCEWSAFSCFQKAQLKSANTG | |
| | | NNERIINVSIKKLKRKPPSTNAGWRQKHRLTCPSCDSYEKKPPKE | |
| | | FLERFKSLLQKMIHQHLSSRTHGSEDS | |
| IL-21_ | 2.04 | MRSSPGNMERIVICLMVIFLGTLVHKSSSQGQDRHMIRMRQLIDI | 387 |
| R119Q | | VDQLKNYVNDLVPEFLPAPEDVETNCEWSAFSCFQKAQLKSANTG | |
| | | NNERIINVSIKKLKRKPPSTNAGRRQKHQLTCPSCDSYEKKPPKE | |
| | | FLERFKSLLQKMIHQHLSSRTHGSEDS | |
| IL-21_ | 2.059 | MRSSPGNMERIVICLMVIFLGTLVHKSSSQGQDRHMIRMLQLIDI | 388 |
| R40L | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.063 | MRSSPGNMERIVICLMVIFLGTLVHKSSSQGQDRNMIRMRQLIDI | 389 |
| H35N | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.064 | MRSSPGNMERIVICLMVIFLGTLVHKSSSQGQDRHMIRMRQLIDI | 390 |
| R114V | | VDQLKNYVNDLVPEFLPAPEDVETNCEWSAFSCFQKAQLKSANTG | |
| | | NNERIINVSIKKLKRKPPSTNAGVRQKHRLTCPSCDSYEKKPPKE | |
| | | FLERFKSLLQKMIHQHLSSRTHGSEDS | |
| IL-21_ | 2.064 | MRSSPGNMERIVICLMVIFLGTLVHKSSSQGQDRHMIRMRQLIDI | 391 |
| K106D | | VDQLKNYVNDLVPEFLPAPEDVETNCEWSAFSCFQKAQLKSANTG | |
| | | NNERIINVSIKKLKRDPPSTNAGRRQKHRLTCPSCDSYEKKPPKE | |
| | | FLERFKSLLQKMIHQHLSSRTHGSEDS | |
| IL-21_ | 2.07 | MRSSPGNMERIVICLMVIFLGTLVHKSSSQGQDRHMIRMRQLIDI | 392 |
| K50S | | VDQLSNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.071 | MRSSPGNMERIVICLMVIFLGTLVHKSSSQGQDRHMIRMRQLIDI | 393 |
| G113E | | VDQLKNYVNDLVPEFLPAPEDVETNCEWSAFSCFQKAQLKSANTG | |
| | | NNERIINVSIKKLKRKPPSTNAERRQKHRLTCPSCDSYEKKPPKE | |
| | | FLERFKSLLQKMIHQHLSSRTHGSEDS | |
| IL-21_ | 2.074 | MRSSPGNMERIVICLMVIFLGTLVHKSSSQGQDRHMIRMRQLIDI | 394 |
| K106E | | VDQLKNYVNDLVPEFLPAPEDVETNCEWSAFSCFQKAQLKSANTG | |
| | | NNERIINVSIKKLKREPPSTNAGRRQKHRLTCPSCDSYEKKPPKE | |
| | | FLERFKSLLQKMIHQHLSSRTHGSEDS | |
| IL-21_ | 2.077 | MRSSPGNMERIVICLMVIFLGTLVHKSSSQGQDRHMIRMRQLIDI | 395 |
| L61Y | | VDQLKNYVNDLVPEFYPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.083 | MRSSPGNMERIVICLMVIFLGTLVHKSSSQGQDRHMIRMRQLIDI | 396 |
| R114Y | | VDQLKNYVNDLVPEFLPAPEDVETNCEWSAFSCFQKAQLKSANTG | |
| | | NNERIINVSIKKLKRKPPSTNAGYRQKHRLTCPSCDSYEKKPPKE | |
| | | FLERFKSLLQKMIHQHLSSRTHGSEDS | |
| IL-21_ | 2.086 | MRSSPGNMERIVICLMVIFLGTLVHKSSSQGQDRHMIRMRQLIDI | 397 |
| K50L | | VDQLLNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.086 | MRSSPGNMERIVICLMVIFLGTLVHKSSSQGQDRHMIRMRQLIDI | 398 |
| F60E | | VDQLKNYVNDLVPEELPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.087 | MRSSPGNMERIVICLMVIFLGTLVHKSSSQGQDRHMIRMRQLIDI | 399 |
| K101T | | VDQLKNYVNDLVPEFLPAPEDVETNCEWSAFSCFQKAQLKSANTG | |
| | | NNERIINVSITKLKRKPPSTNAGRRQKHRLTCPSCDSYEKKPPKE | |
| | | FLERFKSLLQKMIHQHLSSRTHGSEDS | |
| IL-21_ | 2.09 | MRSSPGNMERIVICLMVIFLGTLVHKSSSQGQDRHMIRMRQLIDI | 400 |
| T121D | | VDQLKNYVNDLVPEFLPAPEDVETNCEWSAFSCFQKAQLKSANTG | |
| | | NNERIINVSIKKLKRKPPSTNAGRRQKHRLDCPSCDSYEKKPPKE | |
| | | FLERFKSLLQKMIHQHLSSRTHGSEDS | |
| IL-21_ | 2.09 | MRSSPGNMERIVICLMVIFLGTLVHKSSSQGQDRSMIRMRQLIDI | 401 |
| H35S | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.093 | MRSSPGNMERIVICLMVIFLGTLVHKSSSQGQDRHMIRMRQMIDI | 402 |
| L42M | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.093 | MRSSPGNMERIVICLMVIFLGTLVHKSSSQGQDRHMIRMRQLIDI | 403 |
| R115G | | VDQLKNYVNDLVPEFLPAPEDVETNCEWSAFSCFQKAQLKSANTG | |
| | | NNERIINVSIKKLKRKPPSTNAGRGQKHRLTCPSCDSYEKKPPKE | |
| | | FLERFKSLLQKMIHQHLSSRTHGSEDS | |
| IL-21_ | 2.102 | MRSSPGNMERIVICLMVIFLGTLVHKSSSQGQDRHMIRMRQLIDI | 404 |
| S99A | | VDQLKNYVNDLVPEFLPAPEDVETNCEWSAFSCFQKAQLKSANTG | |
| | | NNERIINVAIKKLKRKPPSTNAGRRQKHRLTCPSCDSYEKKPPKE | |
| | | FLERFKSLLQKMIHQHLSSRTHGSEDS | |
| IL-21_ | 2.117 | MRSSPGNMERIVICLMVIFLGTLVHKSSSQGQDRHLIRMRQLIDI | 405 |
| M36L | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.118 | MRSSPGNMERIVICLMVIFLGTLVHKSSSQGQDRHMIRMRQLIDI | 406 |
| N91E | | VDQLKNYVNDLVPEFLPAPEDVETNCEWSAFSCFQKAQLKSANTG | |
| | | ENERIINVSIKKLKRKPPSTNAGRRQKHRLTCPSCDSYEKKPPKE | |
| | | FLERFKSLLQKMIHQHLSSRTHGSEDS | |
| IL-21_ | 2.121 | MRSSPGNMERIVICLMVIFLGTLVHKSSSQGQDRHMIRMRQLIDI | 407 |
| H149D | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIDQHLSSRTHGSEDS | |
| IL-21_ | 2.122 | MRSSPGNMERIVICLMVIFLGTLVHKSSSQGQDRHMIRMMQLIDI | 408 |
| R40M | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.122 | MRSSPGNMERIVICLMVIFLGTLVHKSSSQGQDRHMIRMRQLIDI | 409 |
| N91P | | VDQLKNYVNDLVPEFLPAPEDVETNCEWSAFSCFQKAQLKSANTG | |
| | | PNERIINVSIKKLKRKPPSTNAGRRQKHRLTCPSCDSYEKKPPKE | |
| | | FLERFKSLLQKMIHQHLSSRTHGSEDS | |
| IL-21_ | 2.122 | MRSSPGNMERIVICLMVIFLGTLVHKSSSQGQDRHMIRMRQLIDI | 410 |
| R115Y | | VDQLKNYVNDLVPEFLPAPEDVETNCEWSAFSCFQKAQLKSANTG | |
| | | NNERIINVSIKKLKRKPPSTNAGRYQKHRLTCPSCDSYEKKPPKE | |
| | | FLERFKSLLQKMIHQHLSSRTHGSEDS | |
| IL-21_ | 2.123 | MRSSPGNMERIVICLMVIFLGTLVHKSSSQGQDRHMIRMRQLIDI | 411 |
| R119Y | | VDQLKNYVNDLVPEFLPAPEDVETNCEWSAFSCFQKAQLKSANTG | |
| | | NNERIINVSIKKLKRKPPSTNAGRRQKHYLTCPSCDSYEKKPPKE | |
| | | FLERFKSLLQKMIHQHLSSRTHGSEDS | |
| IL-21_ | 2.141 | MRSSPGNMERIVICLMVIFLGTLVHKSSSQGQDRHMIRMRQLIDI | 412 |
| N70E | | VDQLKNYVNDLVPEFLPAPEDVETECEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.145 | MRSSPGNMERIVICLMVIFLGTLVHKSSSQGQDRHMIRMRQLIDI | 413 |
| K117Y | | VDQLKNYVNDLVPEFLPAPEDVETNCEWSAFSCFQKAQLKSANTG | |
| | | NNERIINVSIKKLKRKPPSTNAGRRQYHRLTCPSCDSYEKKPPKE | |
| | | FLERFKSLLQKMIHQHLSSRTHGSEDS | |
| IL-21_ | 2.145 | MRSSPGNMERIVICLMVIFLGTLVHKSSSQGQDRHMIRMRQLIDI | 414 |
| P107M | | VDQLKNYVNDLVPEFLPAPEDVETNCEWSAFSCFQKAQLKSANTG | |
| | | NNERIINVSIKKLKRKMPSTNAGRRQKHRLTCPSCDSYEKKPPKE | |
| | | FLERFKSLLQKMIHQHLSSRTHGSEDS | |
| IL-21_ | 2.146 | MRSSPGNMERIVICLMVIFLGTLVHKSSSQGQDRHMIRMQQLIDI | 415 |
| R40Q | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.152 | MRSSPGNMERIVICLMVIFLGTLVHKSSSQGQDRHMIRMRQLIDI | 416 |
| H151D | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQDLSSRTHGSEDS | |
| IL-21_ | 2.163 | MRSSPGNMERIVICLMVIFLGTLVHKSSSQGQDRHMIRMRQLIDI | 417 |
| H118N | | VDQLKNYVNDLVPEFLPAPEDVETNCEWSAFSCFQKAQLKSANTG | |
| | | NNERIINVSIKKLKRKPPSTNAGRRQKNRLTCPSCDSYEKKPPKE | |
| | | FLERFKSLLQKMIHQHLSSRTHGSEDS | |
| IL-21_ | 2.163 | MRSSPGNMERIVICLMVIFLGTLVHKSSSQGQDRHMIRMRQLIDI | 418 |
| T69E | | VDQLKNYVNDLVPEFLPAPEDVEENCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.163 | MRSSPGNMERIVICLMVIFLGTLVHKSSSQGQDRHMIRMRQLIDI | 419 |
| K104Y | | VDQLKNYVNDLVPEFLPAPEDVETNCEWSAFSCFQKAQLKSANTG | |
| | | NNERIINVSIKKLYRKPPSTNAGRRQKHRLTCPSCDSYEKKPPKE | |
| | | FLERFKSLLQKMIHQHLSSRTHGSEDS | |
| IL-21_ | 2.167 | MRSSPGNMERIVICLMVIFLGTLVHKSSSQGQDRHMIRMRQLIDI | 420 |
| R114T | | VDQLKNYVNDLVPEFLPAPEDVETNCEWSAFSCFQKAQLKSANTG | |
| | | NNERIINVSIKKLKRKPPSTNAGTRQKHRLTCPSCDSYEKKPPKE | |
| | | FLERFKSLLQKMIHQHLSSRTHGSEDS | |
| IL-21_ | 2.167 | MRSSPGNMERIVICLMVIFLGTLVHKSSSQGQDRHMIRMRQLIDI | 421 |
| K117S | | VDQLKNYVNDLVPEFLPAPEDVETNCEWSAFSCFQKAQLKSANTG | |
| | | NNERIINVSIKKLKRKPPSTNAGRRQSHRLTCPSCDSYEKKPPKE | |
| | | FLERFKSLLQKMIHQHLSSRTHGSEDS | |
| IL-21_ | 2.168 | MRSSPGNMERIVICLMVIFLGTLVHKSSSQGQDRHMIRMRQLIDI | 422 |
| H118I | | VDQLKNYVNDLVPEFLPAPEDVETNCEWSAFSCFQKAQLKSANTG | |
| | | NNERIINVSIKKLKRKPPSTNAGRRQKIRLTCPSCDSYEKKPPKE | |
| | | FLERFKSLLQKMIHQHLSSRTHGSEDS | |
| IL-21_ | 2.168 | MRSSPGNMERIVICLMVIFLGTLVHKSSSQGQDRHMIRMRQLIDI | 423 |
| S99L | | VDQLKNYVNDLVPEFLPAPEDVETNCEWSAFSCFQKAQLKSANTG | |
| | | NNERIINVLIKKLKRKPPSTNAGRRQKHRLTCPSCDSYEKKPPKE | |
| | | FLERFKSLLQKMIHQHLSSRTHGSEDS | |
| IL-21_ | 2.177 | MRSSPGNMERIVICLMVIFLGTLVHKSSSQGQDRHMIRMRQLIDI | 424 |
| S74E | | VDQLKNYVNDLVPEFLPAPEDVETNCEWEAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.178 | MRSSPGNMERIVICLMVIFLGTLVHKSSSQGQDRHMIRMRQLIDI | 425 |
| Q83P | | VDQLKNYVNDLVPEFLPAPEDVETNCEWSAFSCFQKAPLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.182 | MRSSPGNMERIVICLMVIFLGTLVHKSSSQGQDRHMIRMRQLIDI | 426 |
| K117F | | VDQLKNYVNDLVPEFLPAPEDVETNCEWSAFSCFQKAQLKSANTG | |
| | | NNERIINVSIKKLKRKPPSTNAGRRQFHRLTCPSCDSYEKKPPKE | |
| | | FLERFKSLLQKMIHQHLSSRTHGSEDS | |
| IL-21_ | 2.183 | MRSSPGNMERIVICLMVIFLGTLVHKSSSQGQDRHMIRMRQLIDI | 427 |
| P107I | | VDQLKNYVNDLVPEFLPAPEDVETNCEWSAFSCFQKAQLKSANTG | |
| | | NNERIINVSIKKLKRKIPSTNAGRRQKHRLTCPSCDSYEKKPPKE | |
| | | FLERFKSLLQKMIHQHLSSRTHGSEDS | |
| IL-21_ | 2.183 | MRSSPGNMERIVICLMVIFLGTLVHKSSSQGEDRHMIRMRQLIDI | 428 |
| Q32E | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.186 | MRSSPGNMERIVICLMVIFLGTLVHKSSSQGQDRHMIRMRQLIDI | 429 |
| S99M | | VDQLKNYVNDLVPEFLPAPEDVETNCEWSAFSCFQKAQLKSANTG | |
| | | NNERIINVMIKKLKRKPPSTNAGRRQKHRLTCPSCDSYEKKPPKE | |
| | | FLERFKSLLQKMIHQHLSSRTHGSEDS | |
| IL-21_ | 2.188 | MRSSPGNMERIVICLMVIFLGTLVHKSSSQGQDRHMIRMRQLIDI | 430 |
| T69D | | VDQLKNYVNDLVPEFLPAPEDVEDNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.189 | MRSSPGNMERIVICLMVIFLGTLVHKSSSQGQDRHMIRMRQLIDI | 431 |
| R119S | | VDQLKNYVNDLVPEFLPAPEDVETNCEWSAFSCFQKAQLKSANTG | |
| | | NNERIINVSIKKLKRKPPSTNAGRRQKHSLTCPSCDSYEKKPPKE | |
| | | FLERFKSLLQKMIHQHLSSRTHGSEDS | |
| IL-21_ | 2.196 | MRSSPGNMERIVICLMVIFLGTLVHKSSSQGQDRHMIRMRQLIDI | 432 |
| K117A | | VDQLKNYVNDLVPEFLPAPEDVETNCEWSAFSCFQKAQLKSANTG | |
| | | NNERIINVSIKKLKRKPPSTNAGRRQAHRLTCPSCDSYEKKPPKE | |
| | | FLERFKSLLQKMIHQHLSSRTHGSEDS | |
| IL-21_ | 2.211 | MRSSPGNMERIVICLMVIFLGTLVHKSSSQGQDRHMIRMRQLIDI | 433 |
| F60P | | VDQLKNYVNDLVPEPLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.213 | MRSSPGNMERIVICLMVIFLGTLVHKSSSQGQDRHMIRMYQLIDI | 434 |
| R40Y | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.216 | MRSSPGNMERIVICLMVIFLGTLVHKSSSQGQDRHMIRMRQLIDI | 435 |
| K81L | | VDQLKNYVNDLVPEFLPAPEDVETNCEWSAFSCFQLAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.219 | MRSSPGNMERIVICLMVIFLGTLVHKSSSQGQDRHMIRMRQLIDI | 436 |
| R115T | | VDQLKNYVNDLVPEFLPAPEDVETNCEWSAFSCFQKAQLKSANTG | |
| | | NNERIINVSIKKLKRKPPSTNAGRTQKHRLTCPSCDSYEKKPPKE | |
| | | FLERFKSLLQKMIHQHLSSRTHGSEDS | |
| IL-21_ | 2.221 | MRSSPGNMERIVICLMVIFLGTLVHKSSSQGQDRHMIRMRQLIDI | 437 |
| H118Q | | VDQLKNYVNDLVPEFLPAPEDVETNCEWSAFSCFQKAQLKSANTG | |
| | | NNERIINVSIKKLKRKPPSTNAGRRQKQRLTCPSCDSYEKKPPKE | |
| | | FLERFKSLLQKMIHQHLSSRTHGSEDS | |
| IL-21_ | 2.221 | MRSSPGNMERIVICLMVIFLGTLVHKSSSQGQDRHMIRMRQLIDI | 438 |
| Q83D | | VDQLKNYVNDLVPEFLPAPEDVETNCEWSAFSCFQKADLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.221 | MRSSPGNMERIVICLMVIFLGTLVHKSSSQGQDRIMIRMRQLIDI | 439 |
| H35I | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.225 | MRSSPGNMERIVICLMVIFLGTLVHKSSSQGQDRHMIRMRQLIDI | 440 |
| S86Y | | VDQLKNYVNDLVPEFLPAPEDVETNCEWSAFSCFQKAQLKYANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.225 | MRSSPGNMERIVICLMVIFLGTLVHKSSSQGQDRHMIRMRQLIDI | 441 |
| H118V | | VDQLKNYVNDLVPEFLPAPEDVETNCEWSAFSCFQKAQLKSANTG | |
| | | NNERIINVSIKKLKRKPPSTNAGRRQKVRLTCPSCDSYEKKPPKE | |
| | | FLERFKSLLQKMIHQHLSSRTHGSEDS | |
| IL-21_ | 2.229 | MRSSPGNMERIVICLMVIFLGTLVHKSSSQGQDRMMIRMRQLIDI | 442 |
| H35M | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.233 | MRSSPGNMERIVICLMVIFLGTLVHKSSSQGQDRHMIRMRQLIDI | 443 |
| K104E | | VDQLKNYVNDLVPEFLPAPEDVETNCEWSAFSCFQKAQLKSANTG | |
| | | NNERIINVSIKKLERKPPSTNAGRRQKHRLTCPSCDSYEKKPPKE | |
| | | FLERFKSLLQKMIHQHLSSRTHGSEDS | |
| IL-21_ | 2.234 | MRSSPGNMERIVICLMVIFLGTLVHKSSSQGQDRHMIRMRQLIDI | 444 |
| K104Q | | VDQLKNYVNDLVPEFLPAPEDVETNCEWSAFSCFQKAQLKSANTG | |
| | | NNERIINVSIKKLQRKPPSTNAGRRQKHRLTCPSCDSYEKKPPKE | |
| | | FLERFKSLLQKMIHQHLSSRTHGSEDS | |
| IL-21_ | 2.235 | MRSSPGNMERIVICLMVIFLGTLVHKSSSQGQDRHMIRMRQLIDI | 445 |
| R94G | | VDQLKNYVNDLVPEFLPAPEDVETNCEWSAFSCFQKAQLKSANTG | |
| | | NNEGIINVSIKKLKRKPPSTNAGRRQKHRLTCPSCDSYEKKPPKE | |
| | | FLERFKSLLQKMIHQHLSSRTHGSEDS | |
| IL-21_ | 2.242 | MRSSPGNMERIVICLMVIFLGTLVHKSSSQGQDRHMIRMRQLIDI | 446 |
| W73E | | VDQLKNYVNDLVPEFLPAPEDVETNCEESAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.242 | MRSSPGNMERIVICLMVIFLGTLVHKSSSQGQDRHMIRMRQLIDI | 447 |
| R94Y | | VDQLKNYVNDLVPEFLPAPEDVETNCEWSAFSCFQKAQLKSANTG | |
| | | NNEYIINVSIKKLKRKPPSTNAGRRQKHRLTCPSCDSYEKKPPKE | |
| | | FLERFKSLLQKMIHQHLSSRTHGSEDS | |
| IL-21_ | 2.247 | MRSSPGNMERIVICLMVIFLGTLVHKSSSQGQDRGMIRMRQLIDI | 448 |
| H35G | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.25 | MRSSPGNMERIVICLMVIFLGTLVHKSSSQGQDRHMIRMRQLIDI | 449 |
| L120E | | VDQLKNYVNDLVPEFLPAPEDVETNCEWSAFSCFQKAQLKSANTG | |
| | | NNERIINVSIKKLKRKPPSTNAGRRQKHRETCPSCDSYEKKPPKE | |
| | | FLERFKSLLQKMIHQHLSSRTHGSEDS | |
| IL-21_ | 2.255 | MRSSPGNMERIVICLMVIFLGTLVHKSSSQGQDRLMIRMRQLIDI | 450 |
| H35L | | VDQLKNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.258 | MRSSPGNMERIVICLMVIFLGTLVHKSSSQGQDRHMIRMRQLIDI | 451 |
| R119F | | VDQLKNYVNDLVPEFLPAPEDVETNCEWSAFSCFQKAQLKSANTG | |
| | | NNERIINVSIKKLKRKPPSTNAGRRQKHELTCPSCDSYEKKPPKE | |
| | | FLERFKSLLQKMIHQHLSSRTHGSEDS | |
| IL-21_ | 2.272 | MRSSPGNMERIVICLMVIFLGTLVHKSSSQGQDRHMIRMRQLIDI | 452 |
| P107D | | VDQLKNYVNDLVPEFLPAPEDVETNCEWSAFSCFQKAQLKSANTG | |
| | | NNERIINVSIKKLKRKDPSTNAGRRQKHRLTCPSCDSYEKKPPKE | |
| | | FLERFKSLLQKMIHQHLSSRTHGSEDS | |
| IL-21_ | 2.28 | MRSSPGNMERIVICLMVIFLGTLVHKSSSQGQDRHMIRMRQLIDI | 453 |
| K117P | | VDQLKNYVNDLVPEFLPAPEDVETNCEWSAFSCFQKAQLKSANTG | |
| | | NNERIINVSIKKLKRKPPSTNAGRRQPHRLTCPSCDSYEKKPPKE | |
| | | FLERFKSLLQKMIHQHLSSRTHGSEDS | |
| IL-21_ | 2.288 | MRSSPGNMERIVICLMVIFLGTLVHKSSSQGQDRHMIRMRQLIDI | 454 |
| K117I | | VDQLKNYVNDLVPEFLPAPEDVETNCEWSAFSCFQKAQLKSANTG | |
| | | NNERIINVSIKKLKRKPPSTNAGRRQIHRLTCPSCDSYEKKPPKE | |
| | | FLERFKSLLQKMIHQHLSSRTHGSEDS | |
| IL-21_ | 2.292 | MRSSPGNMERIVICLMVIFLGTLVHKSSSQGQDRHMIRMRQLIDI | 455 |
| N111D | | VDQLKNYVNDLVPEFLPAPEDVETNCEWSAFSCFQKAQLKSANTG | |
| | | NNERIINVSIKKLKRKPPSTDAGRRQKHRLTCPSCDSYEKKPPKE | |
| | | FLERFKSLLQKMIHQHLSSRTHGSEDS | |
| IL-21_ | 2.3 | MRSSPGNMERIVICLMVIFLGTLVHKSSSQGQDRHMIRMRQLIDI | 456 |
| K102F | | VDQLKNYVNDLVPEFLPAPEDVETNCEWSAFSCFQKAQLKSANTG | |
| | | NNERIINVSIKFLKRKPPSTNAGRRQKHRLTCPSCDSYEKKPPKE | |
| | | FLERFKSLLQKMIHQHLSSRTHGSEDS | |
| IL-21_ | 2.308 | MRSSPGNMERIVICLMVIFLGTLVHKSSSQGQDRHMIRMRQLIDI | 457 |
| K50M | | VDQLMNYVNDLVPEFLPAPEDVETNCEWSAFSCFQKAQLKSANTG | |
| | | NNERIINVSIKKLKRKPPSTNAGRRQKHRLTCPSCDSYEKKPPKE | |
| | | FLERFKSLLQKMIHQHLSSRTHGSEDS | |
| IL-21_ | 2.31 | MRSSPGNMERIVICLMVIFLGTLVHKSSSQGQDRHMIRMRQLIDI | 458 |
| K117T | | VDQLKNYVNDLVPEFLPAPEDVETNCEWSAFSCFQKAQLKSANTG | |
| | | NNERIINVSIKKLKRKPPSTNAGRRQTHRLTCPSCDSYEKKPPKE | |
| | | FLERFKSLLQKMIHQHLSSRTHGSEDS | |

TABLE 9
Protein Expression Yields and Purity for Stabilized IL-21-Fc Fusion
Proteins

| | | | | SEQ |
|---|---|---|---|---|
| Variant | | | Predicted | ID |
| Name | Mutations | Amino Acid Sequence | Affinity | NO |
| IL-21_3434 | H35E, N91E, R114L | MRSSPGNMERIVICLMVIFLGTLVHKSSSQGQDREM | 0.541 | 459 |
| | | IRMRQLIDIVDQLKNYVNDLVPEFLPAPEDVETNCE | | |
| | | WSAFSCFQKAQLKSANTGENERIINVSIKKLKRKPP | | |
| | | STNAGLRQKHRLTCPSCDSYEKKPPKEFLERFKSLL | | |
| | | QKMIHQHLSSRTHGSEDS | | |
| IL-21_4257 | H35F, L42D, V57D, | MRSSPGNMERIVICLMVIFLGTLVHKSSSQGQDRFM | 0.572 | 460 |
| | R114Q | IRMRQDIDIVDQLKNYVNDLDPEFLPAPEDVETNCE | | |
| | | WSAFSCFQKAQLKSANTGNNERIINVSIKKLKRKPP | | |
| | | STNAGQRQKHRLTCPSCDSYEKKPPKEFLERFKSLL | | |
| | | QKMIHQHLSSRTHGSEDS | | |
| IL-21_2988 | R94E, R114T | MRSSPGNMERIVICLMVIFLGTLVHKSSSQGQDRHM | 0.617 | 461 |
| | | IRMRQLIDIVDQLKNYVNDLVPEFLPAPEDVETNCE | | |
| | | WSAFSCFQKAQLKSANTGNNEEIINVSIKKLKRKPP | | |
| | | STNAGTRQKHRLTCPSCDSYEKKPPKEFLERFKSLL | | |
| | | QKMIHQHLSSRTHGSEDS | | |
| IL-21_3610 | P107V, R114Q, | MRSSPGNMERIVICLMVIFLGTLVHKSSSQGQDRHM | 0.653 | 462 |
| | R115D | IRMRQLIDIVDQLKNYVNDLVPEFLPAPEDVETNCE | | |
| | | WSAFSCFQKAQLKSANTGNNERIINVSIKKLKRKVP | | |
| | | STNAGQDQKHRLTCPSCDSYEKKPPKEFLERFKSLL | | |
| | | QKMIHQHLSSRTHGSEDS | | |
| IL-21_4177 | H35I, N70G, T110D, | MRSSPGNMERIVICLMVIFLGTLVHKSSSQGQDRIM | 0.663 | 463 |
| | A112P | IRMRQLIDIVDQLKNYVNDLVPEFLPAPEDVETGCE | | |
| | | WSAFSCFQKAQLKSANTGNNERIINVSIKKLKRKPP | | |
| | | SDNPGRRQKHRLTCPSCDSYEKKPPKEFLERFKSLL | | |
| | | QKMIHQHLSSRTHGSEDS | | |
| IL-21_4219 | R94D, G113E, | MRSSPGNMERIVICLMVIFLGTLVHKSSSQGQDRHM | 0.675 | 464 |
| | R119M, T121E | IRMRQLIDIVDQLKNYVNDLVPEFLPAPEDVETNCE | | |
| | | WSAFSCFQKAQLKSANTGNNEDIINVSIKKLKRKPP | | |
| | | STNAERRQKHMLECPSCDSYEKKPPKEFLERFKSLL | | |
| | | QKMIHQHLSSRTHGSEDS | | |
| IL-21_0910 | I45T, K81D, V98E | MRSSPGNMERIVICLMVIFLGTLVHKSSSQGQDRHM | 0.676 | 465 |
| | | IRMRQLIDTVDQLKNYVNDLVPEFLPAPEDVETNCE | | |
| | | WSAFSCFQDAQLKSANTGNNERIINESIKKLKRKPP | | |
| | | STNAGRRQKHRLTCPSCDSYEKKPPKEFLERFKSLL | | |
| | | QKMIHQHLSSRTHGSEDS | | |
| IL-21_2821 | V98D, R115G | MRSSPGNMERIVICLMVIFLGTLVHKSSSQGQDRHM | 0.693 | 466 |
| | | IRMRQLIDIVDQLKNYVNDLVPEFLPAPEDVETNCE | | |
| | | WSAFSCFQKAQLKSANTGNNERIINDSIKKLKRKPP | | |
| | | STNAGRGQKHRLTCPSCDSYEKKPPKEFLERFKSLL | | |
| | | QKMIHQHLSSRTHGSEDS | | |
| IL-21_2897 | Q83E, R114E | MRSSPGNMERIVICLMVIFLGTLVHKSSSQGQDRHM | 0.702 | 467 |
| | | IRMRQLIDIVDQLKNYVNDLVPEFLPAPEDVETNCE | | |
| | | WSAFSCFQKAELKSANTGNNERIINVSIKKLKRKPP | | |
| | | STNAGERQKHRLTCPSCDSYEKKPPKEFLERFKSLL | | |
| | | QKMIHQHLSSRTHGSEDS | | |
| IL-21_3620 | K81E, N111L, | MRSSPGNMERIVICLMVIFLGTLVHKSSSQGQDRHM | 0.711 | 468 |
| | R115G | IRMRQLIDIVDQLKNYVNDLVPEFLPAPEDVETNCE | | |
| | | WSAFSCFQEAQLKSANTGNNERIINVSIKKLKRKPP | | |
| | | STLAGRGQKHRLTCPSCDSYEKKPPKEFLERFKSLL | | |
| | | QKMIHQHLSSRTHGSEDS | | |
| IL-21_4770 | H35F, R40L, E65G, | MRSSPGNMERIVICLMVIFLGTLVHKSSSQGQDRFM | 0.712 | 469 |
| | K106R, R115E | IRMLQLIDIVDQLKNYVNDLVPEFLPAPGDVETNCE | | |
| | | WSAFSCFQKAQLKSANTGNNERIINVSIKKLKRRPP | | |
| | | STNAGREQKHRLTCPSCDSYEKKPPKEFLERFKSLL | | |
| | | QKMIHQHLSSRTHGSEDS | | |
| IL-21_4048 | H35D, Q83G, V98Q, | MRSSPGNMERIVICLMVIFLGTLVHKSSSQGQDRDM | 0.719 | 470 |
| | G113E | IRMRQLIDIVDQLKNYVNDLVPEFLPAPEDVETNCE | | |
| | | WSAFSCFQKAGLKSANTGNNERIINQSIKKLKRKPP | | |
| | | STNAERRQKHRLTCPSCDSYEKKPPKEFLERFKSLL | | |
| | | QKMIHQHLSSRTHGSEDS | | |
| IL-21_4869 | Q32V, W73N, S99A, | MRSSPGNMERIVICLMVIFLGTLVHKSSSQGVDRHM | 0.741 | 471 |
| | K102G, R115D | IRMRQLIDIVDQLKNYVNDLVPEFLPAPEDVETNCE | | |
| | | NSAFSCFQKAQLKSANTGNNERIINVAIKGLKRKPP | | |
| | | STNAGRDQKHRLTCPSCDSYEKKPPKEFLERFKSLL | | |
| | | QKMIHQHLSSRTHGSEDS | | |
| IL-21_4704 | N51I, T69E, P107L, | MRSSPGNMERIVICLMVIFLGTLVHKSSSQGQDRHM | 0.743 | 472 |
| | S109E, R114N | IRMRQLIDIVDQLKIYVNDLVPEFLPAPEDVEENCE | | |
| | | WSAFSCFQKAQLKSANTGNNERIINVSIKKLKRKLP | | |
| | | ETNAGNRQKHRLTCPSCDSYEKKPPKEFLERFKSLL | | |
| | | QKMIHQHLSSRTHGSEDS | | |
| IL-21_4818 | R94E, G113Y, | MRSSPGNMERIVICLMVIFLGTLVHKSSSQGQDRHM | 0.761 | 473 |
| | H118D, L120T, | IRMRQLIDIVDQLKNYVNDLVPEFLPAPEDVETNCE | | |
| | T121Y | WSAFSCFQKAQLKSANTGNNEEIINVSIKKLKRKPP | | |
| | | STNAYRRQKDRTYCPSCDSYEKKPPKEFLERFKSLL | | |
| | | QKMIHQHLSSRTHGSEDS | | |
| IL-21_4778 | R94P, P107V, | MRSSPGNMERIVICLMVIFLGTLVHKSSSQGQDRHM | 0.795 | 474 |
| | S109L, R114I, | IRMRQLIDIVDQLKNYVNDLVPEFLPAPEDVETNCE | | |
| | H118E | WSAFSCFQKAQLKSANTGNNEPIINVSIKKLKRKVP | | |
| | | LTNAGIRQKERLTCPSCDSYEKKPPKEFLERFKSLL | | |
| | | QKMIHQHLSSRTHGSEDS | | |
| IL-21_4297 | H35Y, W73S, | MRSSPGNMERIVICLMVIFLGTLVHKSSSQGQDRYM | 0.808 | 475 |
| | T110V, R115D | IRMRQLIDIVDQLKNYVNDLVPEFLPAPEDVETNCE | | |
| | | SSAFSCFQKAQLKSANTGNNERIINVSIKKLKRKPP | | |
| | | SVNAGRDQKHRLTCPSCDSYEKKPPKEFLERFKSLL | | |
| | | QKMIHQHLSSRTHGSEDS | | |
| IL-21_2857 | N111E, R115D | MRSSPGNMERIVICLMVIFLGTLVHKSSSQGQDRHM | 0.818 | 476 |
| | | IRMRQLIDIVDQLKNYVNDLVPEFLPAPEDVETNCE | | |
| | | WSAFSCFQKAQLKSANTGNNERIINVSIKKLKRKPP | | |
| | | STEAGRDQKHRLTCPSCDSYEKKPPKEFLERFKSLL | | |
| | | QKMIHQHLSSRTHGSEDS | | |
| IL-21_3105 | K81T, R115E | MRSSPGNMERIVICLMVIFLGTLVHKSSSQGQDRHM | 0.841 | 477 |
| | | IRMRQLIDIVDQLKNYVNDLVPEFLPAPEDVETNCE | | |
| | | WSAFSCFQTAQLKSANTGNNERIINVSIKKLKRKPP | | |
| | | STNAGREQKHRLTCPSCDSYEKKPPKEFLERFKSLL | | |
| | | QKMIHQHLSSRTHGSEDS | | |
| IL-21_4098 | H35T, V57K, | MRSSPGNMERIVICLMVIFLGTLVHKSSSQGQDRTM | 0.865 | 478 |
| | A112D, R114E | IRMRQLIDIVDQLKNYVNDLKPEFLPAPEDVETNCE | | |
| | | WSAFSCFQKAQLKSANTGNNERIINVSIKKLKRKPP | | |
| | | STNDGERQKHRLTCPSCDSYEKKPPKEFLERFKSLL | | |
| | | QKMIHQHLSSRTHGSEDS | | |
| IL-21_2954 | R94E, P107L | MRSSPGNMERIVICLMVIFLGTLVHKSSSQGQDRHM | 0.878 | 479 |
| | | IRMRQLIDIVDQLKNYVNDLVPEFLPAPEDVETNCE | | |
| | | WSAFSCFQKAQLKSANTGNNEEIINVSIKKLKRKLP | | |
| | | STNAGRRQKHRLTCPSCDSYEKKPPKEFLERFKSLL | | |
| | | QKMIHQHLSSRTHGSEDS | | |
| IL-21_3581 | R114V, R115E, | MRSSPGNMERIVICLMVIFLGTLVHKSSSQGQDRHM | 0.891 | 480 |
| | K117A | IRMRQLIDIVDQLKNYVNDLVPEFLPAPEDVETNCE | | |
| | | WSAFSCFQKAQLKSANTGNNERIINVSIKKLKRKPP | | |
| | | STNAGVEQAHRLTCPSCDSYEKKPPKEFLERFKSLL | | |
| | | QKMIHQHLSSRTHGSEDS | | |
| IL-21_4274 | N91Q, K102Q, | MRSSPGNMERIVICLMVIFLGTLVHKSSSQGQDRHM | 0.893 | 481 |
| | T110D, T121L | IRMRQLIDIVDQLKNYVNDLVPEFLPAPEDVETNCE | | |
| | | WSAFSCFQKAQLKSANTGQNERIINVSIKQLKRKPP | | |
| | | SDNAGRRQKHRLLCPSCDSYEKKPPKEFLERFKSLL | | |
| | | QKMIHQHLSSRTHGSEDS | | |
| IL-21_3517 | R40V, N70D, R114G | MRSSPGNMERIVICLMVIFLGTLVHKSSSQGQDRHM | 0.911 | 482 |
| | | IRMVQLIDIVDQLKNYVNDLVPEFLPAPEDVETDCE | | |
| | | WSAFSCFQKAQLKSANTGNNERIINVSIKKLKRKPP | | |
| | | STNAGGRQKHRLTCPSCDSYEKKPPKEFLERFKSLL | | |
| | | QKMIHQHLSSRTHGSEDS | | |
| IL-21_4159 | R34S, R94E, S109A, | MRSSPGNMERIVICLMVIFLGTLVHKSSSQGQDSHM | 0.912 | 483 |
| | R115I | IRMRQLIDIVDQLKNYVNDLVPEFLPAPEDVETNCE | | |
| | | WSAFSCFQKAQLKSANTGNNEEIINVSIKKLKRKPP | | |
| | | ATNAGRIQKHRLTCPSCDSYEKKPPKEFLERFKSLL | | |
| | | QKMIHQHLSSRTHGSEDS | | |
| IL-21_2178 | Q48S, K50E, E68I, | MRSSPGNMERIVICLMVIFLGTLVHKSSSQGQDRHM | 0.922 | 484 |
| | K102Q, R114D | IRMRQLIDIVDSLENYVNDLVPEFLPAPEDVITNCE | | |
| | | WSAFSCFQKAQLKSANTGNNERIINVSIKQLKRKPP | | |
| | | STNAGDRQKHRLTCPSCDSYEKKPPKEFLERFKSLL | | |
| | | QKMIHQHLSSRTHGSEDS | | |
| IL-21_2977 | R94N, H118E | MRSSPGNMERIVICLMVIFLGTLVHKSSSQGQDRHM | 0.929 | 485 |
| | | IRMRQLIDIVDQLKNYVNDLVPEFLPAPEDVETNCE | | |
| | | WSAFSCFQKAQLKSANTGNNENIINVSIKKLKRKPP | | |
| | | STNAGRRQKERLTCPSCDSYEKKPPKEFLERFKSLL | | |
| | | QKMIHQHLSSRTHGSEDS | | |
| IL-21_3027 | K81T, K102Q | MRSSPGNMERIVICLMVIFLGTLVHKSSSQGQDRHM | 0.933 | 486 |
| | | IRMRQLIDIVDQLKNYVNDLVPEFLPAPEDVETNCE | | |
| | | WSAFSCFQTAQLKSANTGNNERIINVSIKQLKRKPP | | |
| | | STNAGRRQKHRLTCPSCDSYEKKPPKEFLERFKSLL | | |
| | | QKMIHQHLSSRTHGSEDS | | |
| IL-21_4642 | R94T, R114T, | MRSSPGNMERIVICLMVIFLGTLVHKSSSQGQDRHM | 0.94 | 487 |
| | R115E, D126E, | IRMRQLIDIVDQLKNYVNDLVPEFLPAPEDVETNCE | | |
| | E135V | WSAFSCFQKAQLKSANTGNNETIINVSIKKLKRKPP | | |
| | | STNAGTEQKHRLTCPSCESYEKKPPKVFLERFKSLL | | |
| | | QKMIHQHLSSRTHGSEDS | | |
| IL-21_3580 | Q32G, N111E, | MRSSPGNMERIVICLMVIFLGTLVHKSSSQGGDRHM | 0.971 | 488 |
| | R115P | IRMRQLIDIVDQLKNYVNDLVPEFLPAPEDVETNCE | | |
| | | WSAFSCFQKAQLKSANTGNNERIINVSIKKLKRKPP | | |
| | | STEAGRPQKHRLTCPSCDSYEKKPPKEFLERFKSLL | | |
| | | QKMIHQHLSSRTHGSEDS | | |
| IL-21_2801 | R114I, R115G | MRSSPGNMERIVICLMVIFLGTLVHKSSSQGQDRHM | 0.976 | 489 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGIGQKHRLTCPSCDSYEKKPPKEFLERFKSLL | | |
| | | QKMIHQHLSSRTHGSEDS | | |
| IL-21_2773 | N70E, R114G | MRSSPGNMERIVICLMVIFLGTLVHKSSSQGQDRHM | 0.976 | 490 |
| | | IRMRQLIDIVDQLKNYVNDLVPEFLPAPEDVETECE | | |
| | | WSAFSCFQKAQLKSANTGNNERIINVSIKKLKRKPP | | |
| | | STNAGGRQKHRLTCPSCDSYEKKPPKEFLERFKSLL | | |
| | | QKMIHQHLSSRTHGSEDS | | |
| IL-21_4663 | H35F, R40Y, I45V, | MRSSPGNMERIVICLMVIFLGTLVHKSSSQGQDRFM | 0.983 | 491 |
| | T69D, T89A | IRMYQLIDVVDQLKNYVNDLVPEFLPAPEDVEDNCE | | |
| | | WSAFSCFQKAQLKSANAGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3997 | H35E, N51I, T69D, | MRSSPGNMERIVICLMVIFLGTLVHKSSSQGQDREM | 0.985 | 492 |
| | R115K | IRMRQLIDIVDQLKIYVNDLVPEFLPAPEDVEDNCE | | |
| | | WSAFSCFQKAQLKSANTGNNERIINVSIKKLKRKPP | | |
| | | STNAGRKQKHRLTCPSCDSYEKKPPKEFLERFKSLL | | |
| | | QKMIHQHLSSRTHGSEDS | | |
| IL-21_3675 | K101S, R114A, | MRSSPGNMERIVICLMVIFLGTLVHKSSSQGQDRHM | 0.996 | 493 |
| | R119H | IRMRQLIDIVDQLKNYVNDLVPEFLPAPEDVETNCE | | |
| | | WSAFSCFQKAQLKSANTGNNERIINVSISKLKRKPP | | |
| | | STNAGARQKHHLTCPSCDSYEKKPPKEFLERFKSLL | | |
| | | QKMIHQHLSSRTHGSEDS | | |
| IL-21_2898 | M36L, R115D | MRSSPGNMERIVICLMVIFLGTLVHKSSSQGQDRHL | 1.001 | 494 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRDQKHRLTCPSCDSYEKKPPKEFLERFKSLL | | |
| | | QKMIHQHLSSRTHGSEDS | | |
| IL-21_4720 | H35A, E72Y, | MRSSPGNMERIVICLMVIFLGTLVHKSSSQGQDRAM | 1.008 | 495 |
| | A112G, G113E, | IRMRQLIDIVDQLKNYVNDLVPEFLPAPEDVETNCY | | |
| | R114Y | WSAFSCFQKAQLKSANTGNNERIINVSIKKLKRKPP | | |
| | | STNGEYRQKHRLTCPSCDSYEKKPPKEFLERFKSLL | | |
| | | QKMIHQHLSSRTHGSEDS | | |
| IL-21_2930 | H35E, G113D | MRSSPGNMERIVICLMVIFLGTLVHKSSSQGQDREM | 1.011 | 496 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNADRRQKHRLTCPSCDSYEKKPPKEFLERFKSLL | | |
| | | QKMIHQHLSSRTHGSEDS | | |
| IL-21_4047 | H35E, T69E, K85Q, | MRSSPGNMERIVICLMVIFLGTLVHKSSSQGQDREM | 1.027 | 497 |
| | G113A | IRMRQLIDIVDQLKNYVNDLVPEFLPAPEDVEENCE | | |
| | | WSAFSCFQKAQLQSANTGNNERIINVSIKKLKRKPP | | |
| | | STNAARRQKHRLTCPSCDSYEKKPPKEFLERFKSLL | | |
| | | QKMIHQHLSSRTHGSEDS | | |
| IL-21_2812 | R94S, R114A | MRSSPGNMERIVICLMVIFLGTLVHKSSSQGQDRHM | 1.042 | 498 |
| | | IRMRQLIDIVDQLKNYVNDLVPEFLPAPEDVETNCE | | |
| | | WSAFSCFQKAQLKSANTGNNESIINVSIKKLKRKPP | | |
| | | STNAGARQKHRLTCPSCDSYEKKPPKEFLERFKSLL | | |
| | | QKMIHQHLSSRTHGSEDS | | |
| IL-21_4286 | 196V, S99T, T110D, | MRSSPGNMERIVICLMVIFLGTLVHKSSSQGQDRHM | 1.07 | 499 |
| | R114E | IRMRQLIDIVDQLKNYVNDLVPEFLPAPEDVETNCE | | |
| | | WSAFSCFQKAQLKSANTGNNERIVNVTIKKLKRKPP | | |
| | | SDNAGERQKHRLTCPSCDSYEKKPPKEFLERFKSLL | | |
| | | QKMIHQHLSSRTHGSEDS | | |
| IL-21_4131 | Q32E, R38Y, | MRSSPGNMERIVICLMVIFLGTLVHKSSSQGEDRHM | 1.071 | 500 |
| | K102Q, N111V | IYMRQLIDIVDQLKNYVNDLVPEFLPAPEDVETNCE | | |
| | | WSAFSCFQKAQLKSANTGNNERIINVSIKQLKRKPP | | |
| | | STVAGRRQKHRLTCPSCDSYEKKPPKEFLERFKSLL | | |
| | | QKMIHQHLSSRTHGSEDS | | |
| IL-21_2715 | R114T, R115E | MRSSPGNMERIVICLMVIFLGTLVHKSSSQGQDRHM | 1.073 | 501 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGTEQKHRLTCPSCDSYEKKPPKEFLERFKSLL | | |
| | | QKMIHQHLSSRTHGSEDS | | |
| IL-21_4188 | N70A, R94N, | MRSSPGNMERIVICLMVIFLGTLVHKSSSQGQDRHM | 1.078 | 502 |
| | P107L, R119T | IRMRQLIDIVDQLKNYVNDLVPEFLPAPEDVETACE | | |
| | | WSAFSCFQKAQLKSANTGNNENIINVSIKKLKRKLP | | |
| | | STNAGRRQKHTLTCPSCDSYEKKPPKEFLERFKSLL | | |
| | | QKMIHQHLSSRTHGSEDS | | |
| IL-21_3569 | H35E, M36Q, N70D | MRSSPGNMERIVICLMVIFLGTLVHKSSSQGQDREQ | 1.08 | 503 |
| | | IRMRQLIDIVDQLKNYVNDLVPEFLPAPEDVETDCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3394 | R114I, R115G, | MRSSPGNMERIVICLMVIFLGTLVHKSSSQGQDRHM | 1.085 | 504 |
| | H118G | IRMRQLIDIVDQLKNYVNDLVPEFLPAPEDVETNCE | | |
| | | WSAFSCFQKAQLKSANTGNNERIINVSIKKLKRKPP | | |
| | | STNAGIGQKGRLTCPSCDSYEKKPPKEFLERFKSLL | | |
| | | QKMIHQHLSSRTHGSEDS | | |
| IL-21_3338 | H35T, M36A, R115P | MRSSPGNMERIVICLMVIFLGTLVHKSSSQGQDRTA | 1.109 | 505 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRPQKHRLTCPSCDSYEKKPPKEFLERFKSLL | | |
| | | QKMIHQHLSSRTHGSEDS | | |
| IL-21_2846 | R94T, R119E | MRSSPGNMERIVICLMVIFLGTLVHKSSSQGQDRHM | 1.109 | 506 |
| | | IRMRQLIDIVDQLKNYVNDLVPEFLPAPEDVETNCE | | |
| | | WSAFSCFQKAQLKSANTGNNETIINVSIKKLKRKPP | | |
| | | STNAGRRQKHELTCPSCDSYEKKPPKEFLERFKSLL | | |
| | | QKMIHQHLSSRTHGSEDS | | |
| IL-21_1637 | N51H, V57E, N70E, | MRSSPGNMERIVICLMVIFLGTLVHKSSSQGQDRHM | 1.114 | 507 |
| | K101E | IRMRQLIDIVDQLKHYVNDLEPEFLPAPEDVETECE | | |
| | | WSAFSCFQKAQLKSANTGNNERIINVSIEKLKRKPP | | |
| | | STNAGRRQKHRLTCPSCDSYEKKPPKEFLERFKSLL | | |
| | | QKMIHQHLSSRTHGSEDS | | |
| IL-21_4232 | Q32S, R34T, F76L, | MRSSPGNMERIVICLMVIFLGTLVHKSSSQGSDTHM | 1.118 | 508 |
| | V98D | IRMRQLIDIVDQLKNYVNDLVPEFLPAPEDVETNCE | | |
| | | WSALSCFQKAQLKSANTGNNERIINDSIKKLKRKPP | | |
| | | STNAGRRQKHRLTCPSCDSYEKKPPKEFLERFKSLL | | |
| | | QKMIHQHLSSRTHGSEDS | | |
| IL-21_3556 | N91E, K101S, | MRSSPGNMERIVICLMVIFLGTLVHKSSSQGQDRHM | 1.144 | 509 |
| | N111S | IRMRQLIDIVDQLKNYVNDLVPEFLPAPEDVETNCE | | |
| | | WSAFSCFQKAQLKSANTGENERIINVSISKLKRKPP | | |
| | | STSAGRRQKHRLTCPSCDSYEKKPPKEFLERFKSLL | | |
| | | QKMIHQHLSSRTHGSEDS | | |
| IL-21_3622 | S99T, H118S, | MRSSPGNMERIVICLMVIFLGTLVHKSSSQGQDRHM | 1.149 | 510 |
| | R119D | IRMRQLIDIVDQLKNYVNDLVPEFLPAPEDVETNCE | | |
| | | WSAFSCFQKAQLKSANTGNNERIINVTIKKLKRKPP | | |
| | | STNAGRRQKSDLTCPSCDSYEKKPPKEFLERFKSLL | | |
| | | QKMIHQHLSSRTHGSEDS | | |
| IL-21_3516 | A112M, G113D, | MRSSPGNMERIVICLMVIFLGTLVHKSSSQGQDRHM | 1.152 | 511 |
| | R114T | IRMRQLIDIVDQLKNYVNDLVPEFLPAPEDVETNCE | | |
| | | WSAFSCFQKAQLKSANTGNNERIINVSIKKLKRKPP | | |
| | | STNMDTRQKHRLTCPSCDSYEKKPPKEFLERFKSLL | | |
| | | QKMIHQHLSSRTHGSEDS | | |
| IL-21_2667 | Q32M, R115D | MRSSPGNMERIVICLMVIFLGTLVHKSSSQGMDRHM | 1.156 | 512 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRDQKHRLTCPSCDSYEKKPPKEFLERFKSLL | | |
| | | QKMIHQHLSSRTHGSEDS | | |
| IL-21_3642 | H35D, P107I, S109I | MRSSPGNMERIVICLMVIFLGTLVHKSSSQGQDRDM | 1.158 | 513 |
| | | IRMRQLIDIVDQLKNYVNDLVPEFLPAPEDVETNCE | | |
| | | WSAFSCFQKAQLKSANTGNNERIINVSIKKLKRKIP | | |
| | | ITNAGRRQKHRLTCPSCDSYEKKPPKEFLERFKSLL | | |
| | | QKMIHQHLSSRTHGSEDS | | |
| IL-21_3306 | H35T, M39L, V98T | MRSSPGNMERIVICLMVIFLGTLVHKSSSQGQDRTM | 1.174 | 514 |
| | | IRLRQLIDIVDQLKNYVNDLVPEFLPAPEDVETNCE | | |
| | | WSAFSCFQKAQLKSANTGNNERIINTSIKKLKRKPP | | |
| | | STNAGRRQKHRLTCPSCDSYEKKPPKEFLERFKSLL | | |
| | | QKMIHQHLSSRTHGSEDS | | |
| IL-21_2949 | Q48D, R114A | MRSSPGNMERIVICLMVIFLGTLVHKSSSQGQDRHM | 1.176 | 515 |
| | | IRMRQLIDIVDDLKNYVNDLVPEFLPAPEDVETNCE | | |
| | | WSAFSCFQKAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_3056 | R115E, L120E | MRSSPGNMERIVICLMVIFLGTLVHKSSSQGQDRHM | 1.178 | 516 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGREQKHRETCPSCDSYEKKPPKEFLERFKSLL | | |
| | | QKMIHQHLSSRTHGSEDS | | |
| IL-21_4628 | W73F, P107V, | MRSSPGNMERIVICLMVIFLGTLVHKSSSQGQDRHM | 1.181 | 517 |
| | R114K, H118D, | IRMRQLIDIVDQLKNYVNDLVPEFLPAPEDVETNCE | | |
| | R119N | FSAFSCFQKAQLKSANTGNNERIINVSIKKLKRKVP | | |
| | | STNAGKRQKDNLTCPSCDSYEKKPPKEFLERFKSLL | | |
| | | QKMIHQHLSSRTHGSEDS | | |
| IL-21_2704 | R114V, R115G | MRSSPGNMERIVICLMVIFLGTLVHKSSSQGQDRHM | 1.191 | 518 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGVGQKHRLTCPSCDSYEKKPPKEFLERFKSLL | | |
| | | QKMIHQHLSSRTHGSEDS | | |
| IL-21_3074 | E68K, V98D | MRSSPGNMERIVICLMVIFLGTLVHKSSSQGQDRHM | 1.193 | 519 |
| | | IRMRQLIDIVDQLKNYVNDLVPEFLPAPEDVKTNCE | | |
| | | WSAFSCFQKAQLKSANTGNNERIINDSIKKLKRKPP | | |
| | | STNAGRRQKHRLTCPSCDSYEKKPPKEFLERFKSLL | | |
| | | QKMIHQHLSSRTHGSEDS | | |
| IL-21_2809 | N111D, R114G | MRSSPGNMERIVICLMVIFLGTLVHKSSSQGQDRHM | 1.194 | 520 |
| | | IRMRQLIDIVDQLKNYVNDLVPEFLPAPEDVETNCE | | |
| | | WSAFSCFQKAQLKSANTGNNERIINVSIKKLKRKPP | | |
| | | STDAGGRQKHRLTCPSCDSYEKKPPKEFLERFKSLL | | |
| | | QKMIHQHLSSRTHGSEDS | | |
| IL-21_2959 | F60L, R115D | MRSSPGNMERIVICLMVIFLGTLVHKSSSQGQDRHM | 1.198 | 521 |
| | | IRMRQLIDIVDQLKNYVNDLVPELLPAPEDVETNCE | | |
| | | WSAFSCFQKAQLKSANTGNNERIINVSIKKLKRKPP | | |
| | | STNAGRDQKHRLTCPSCDSYEKKPPKEFLERFKSLL | | |
| | | QKMIHQHLSSRTHGSEDS | | |
| IL-21_3454 | H35S, S77A, V98M | MRSSPGNMERIVICLMVIFLGTLVHKSSSQGQDRSM | 1.199 | 522 |
| | | IRMRQLIDIVDQLKNYVNDLVPEFLPAPEDVETNCE | | |
| | | WSAFACFQKAQLKSANTGNNERIINMSIKKLKRKPP | | |
| | | STNAGRRQKHRLTCPSCDSYEKKPPKEFLERFKSLL | | |
| | | QKMIHQHLSSRTHGSEDS | | |
| IL-21_3399 | E72L, R114A, | MRSSPGNMERIVICLMVIFLGTLVHKSSSQGQDRHM | 1.199 | 523 |
| | R119N | IRMRQLIDIVDQLKNYVNDLVPEFLPAPEDVETNCL | | |
| | | WSAFSCFQKAQLKSANTGNNERIINVSIKKLKRKPP | | |
| | | STNAGARQKHNLTCPSCDSYEKKPPKEFLERFKSLL | | |
| | | QKMIHQHLSSRTHGSEDS | | |
| IL-21_4290 | T69E, K101L, | MRSSPGNMERIVICLMVIFLGTLVHKSSSQGQDRHM | 1.201 | 524 |
| | N111E, G113S | IRMRQLIDIVDQLKNYVNDLVPEFLPAPEDVEENCE | | |
| | | WSAFSCFQKAQLKSANTGNNERIINVSILKLKRKPP | | |
| | | STEASRRQKHRLTCPSCDSYEKKPPKEFLERFKSLL | | |
| | | QKMIHQHLSSRTHGSEDS | | |
| IL-21_3109 | Q83E, S109P | MRSSPGNMERIVICLMVIFLGTLVHKSSSQGQDRHM | 1.203 | 525 |
| | | IRMRQLIDIVDQLKNYVNDLVPEFLPAPEDVETNCE | | |
| | | WSAFSCFQKAELKSANTGNNERIINVSIKKLKRKPP | | |
| | | PTNAGRRQKHRLTCPSCDSYEKKPPKEFLERFKSLL | | |
| | | QKMIHQHLSSRTHGSEDS | | |
| IL-21_4721 | R94Q, N111M, | MRSSPGNMERIVICLMVIFLGTLVHKSSSQGQDRHM | 1.204 | 526 |
| | A112E, G113R, | IRMRQLIDIVDQLKNYVNDLVPEFLPAPEDVETNCE | | |
| | R115Y | WSAFSCFQKAQLKSANTGNNEQIINVSIKKLKRKPP | | |
| | | STMERRYQKHRLTCPSCDSYEKKPPKEFLERFKSLL | | |
| | | QKMIHQHLSSRTHGSEDS | | |
| IL-21_3647 | N70H, R94A, | MRSSPGNMERIVICLMVIFLGTLVHKSSSQGQDRHM | 1.213 | 527 |
| | A112D | IRMRQLIDIVDQLKNYVNDLVPEFLPAPEDVETHCE | | |
| | | WSAFSCFQKAQLKSANTGNNEAIINVSIKKLKRKPP | | |
| | | STNDGRRQKHRLTCPSCDSYEKKPPKEFLERFKSLL | | |
| | | QKMIHQHLSSRTHGSEDS | | |
| IL-21_3632 | H35E, M36G, | MRSSPGNMERIVICLMVIFLGTLVHKSSSQGQDREG | 1.216 | 528 |
| | K106T | IRMRQLIDIVDQLKNYVNDLVPEFLPAPEDVETNCE | | |
| | | WSAFSCFQKAQLKSANTGNNERIINVSIKKLKRTPP | | |
| | | STNAGRRQKHRLTCPSCDSYEKKPPKEFLERFKSLL | | |
| | | QKMIHQHLSSRTHGSEDS | | |
| IL-21_2882 | K81N, N111P | MRSSPGNMERIVICLMVIFLGTLVHKSSSQGQDRHM | 1.219 | 529 |
| | | IRMRQLIDIVDQLKNYVNDLVPEFLPAPEDVETNCE | | |
| | | WSAFSCFQNAQLKSANTGNNERIINVSIKKLKRKPP | | |
| | | STPAGRRQKHRLTCPSCDSYEKKPPKEFLERFKSLL | | |
| | | QKMIHQHLSSRTHGSEDS | | |
| IL-21_3709 | Q32I, R40L, K81S | MRSSPGNMERIVICLMVIFLGTLVHKSSSQGIDRHM | 1.223 | 530 |
| | | IRMLQLIDIVDQLKNYVNDLVPEFLPAPEDVETNCE | | |
| | | WSAFSCFQSAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3426 | H35S, N97D, N111I | MRSSPGNMERIVICLMVIFLGTLVHKSSSQGQDRSM | 1.225 | 531 |
| | | IRMRQLIDIVDQLKNYVNDLVPEFLPAPEDVETNCE | | |
| | | WSAFSCFQKAQLKSANTGNNERIIDVSIKKLKRKPP | | |
| | | STIAGRRQKHRLTCPSCDSYEKKPPKEFLERFKSLL | | |
| | | QKMIHQHLSSRTHGSEDS | | |
| IL-21_4887 | H35N, I37F, R40Q, | MRSSPGNMERIVICLMVIFLGTLVHKSSSQGQDRNM | 1.226 | 532 |
| | E65S, R115S | FRMQQLIDIVDQLKNYVNDLVPEFLPAPSDVETNCE | | |
| | | WSAFSCFQKAQLKSANTGNNERIINVSIKKLKRKPP | | |
| | | STNAGRSQKHRLTCPSCDSYEKKPPKEFLERFKSLL | | |
| | | QKMIHQHLSSRTHGSEDS | | |
| IL-21_2841 | N70G, Q83E | MRSSPGNMERIVICLMVIFLGTLVHKSSSQGQDRHM | 1.228 | 533 |
| | | IRMRQLIDIVDQLKNYVNDLVPEFLPAPEDVETGCE | | |
| | | WSAFSCFQKAELKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3367 | T89S, S99A, R114G | MRSSPGNMERIVICLMVIFLGTLVHKSSSQGQDRHM | 1.235 | 534 |
| | | IRMRQLIDIVDQLKNYVNDLVPEFLPAPEDVETNCE | | |
| | | WSAFSCFQKAQLKSANSGNNERIINVAIKKLKRKPP | | |
| | | STNAGGRQKHRLTCPSCDSYEKKPPKEFLERFKSLL | | |
| | | QKMIHQHLSSRTHGSEDS | | |
| IL-21_2726 | N70G, R114G | MRSSPGNMERIVICLMVIFLGTLVHKSSSQGQDRHM | 1.238 | 535 |
| | | IRMRQLIDIVDQLKNYVNDLVPEFLPAPEDVETGCE | | |
| | | WSAFSCFQKAQLKSANTGNNERIINVSIKKLKRKPP | | |
| | | STNAGGRQKHRLTCPSCDSYEKKPPKEFLERFKSLL | | |
| | | QKMIHQHLSSRTHGSEDS | | |
| IL-21_0403 | K50G, R94E | MRSSPGNMERIVICLMVIFLGTLVHKSSSQGQDRHM | 1.241 | 536 |
| | | IRMRQLIDIVDQLGNYVNDLVPEFLPAPEDVETNCE | | |
| | | WSAFSCFQKAQLKSANTGNNEEIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0713 | I45G, P58Q, V98E | MRSSPGNMERIVICLMVIFLGTLVHKSSSQGQDRHM | 1.243 | 537 |
| | | IRMRQLIDGVDQLKNYVNDLVQEFLPAPEDVETNCE | | |
| | | WSAFSCFQKAQLKSANTGNNERIINESIKKLKRKPP | | |
| | | STNAGRRQKHRLTCPSCDSYEKKPPKEFLERFKSLL | | |
| | | QKMIHQHLSSRTHGSEDS | | |
| IL-21_2660 | M39L, R115G | MRSSPGNMERIVICLMVIFLGTLVHKSSSQGQDRHM | 1.246 | 538 |
| | | IRLRQLIDIVDQLKNYVNDLVPEFLPAPEDVETNCE | | |
| | | WSAFSCFQKAQLKSANTGNNERIINVSIKKLKRKPP | | |
| | | STNAGRGQKHRLTCPSCDSYEKKPPKEFLERFKSLL | | |
| | | QKMIHQHLSSRTHGSEDS | | |
| IL-21_4742 | N70H, W73D, S86V, | MRSSPGNMERIVICLMVIFLGTLVHKSSSQGQDRHM | 1.246 | 539 |
| | T89A, K106F | IRMRQLIDIVDQLKNYVNDLVPEFLPAPEDVETHCE | | |
| | | DSAFSCFQKAQLKVANAGNNERIINVSIKKLKREPP | | |
| | | STNAGRRQKHRLTCPSCDSYEKKPPKEFLERFKSLL | | |
| | | QKMIHQHLSSRTHGSEDS | | |
| IL-21_4123 | A112E, H118G, | MRSSPGNMERIVICLMVIFLGTLVHKSSSQGQDRHM | 1.248 | 540 |
| | R119T, T121N | IRMRQLIDIVDQLKNYVNDLVPEFLPAPEDVETNCE | | |
| | | WSAFSCFQKAQLKSANTGNNERIINVSIKKLKRKPP | | |
| | | STNEGRRQKGTLNCPSCDSYEKKPPKEFLERFKSLL | | |
| | | QKMIHQHLSSRTHGSEDS | | |
| IL-21_4823 | H35F, M36L, V53E, | MRSSPGNMERIVICLMVIFLGTLVHKSSSQGQDREL | 1.253 | 541 |
| | K106P, R115A | IRMRQLIDIVDQLKNYENDLVPEFLPAPEDVETNCE | | |
| | | WSAFSCFQKAQLKSANTGNNERIINVSIKKLKRPPP | | |
| | | STNAGRAQKHRLTCPSCDSYEKKPPKEFLERFKSLL | | |
| | | QKMIHQHLSSRTHGSEDS | | |
| IL-21_3363 | R114G, H118G, | MRSSPGNMERIVICLMVIFLGTLVHKSSSQGQDRHM | 1.254 | 542 |
| | T121E | IRMRQLIDIVDQLKNYVNDLVPEFLPAPEDVETNCE | | |
| | | WSAFSCFQKAQLKSANTGNNERIINVSIKKLKRKPP | | |
| | | STNAGGRQKGRLECPSCDSYEKKPPKEFLERFKSLL | | |
| | | QKMIHQHLSSRTHGSEDS | | |
| IL-21_0749 | K50S, P107T, | MRSSPGNMERIVICLMVIFLGTLVHKSSSQGQDRHM | 1.256 | 543 |
| | R114V | IRMRQLIDIVDQLSNYVNDLVPEFLPAPEDVETNCE | | |
| | | WSAFSCFQKAQLKSANTGNNERIINVSIKKLKRKTP | | |
| | | STNAGVRQKHRLTCPSCDSYEKKPPKEFLERFKSLL | | |
| | | QKMIHQHLSSRTHGSEDS | | |
| IL-21_2755 | R40M, R114A | MRSSPGNMERIVICLMVIFLGTLVHKSSSQGQDRHM | 1.259 | 544 |
| | | IRMMQLIDIVDQLKNYVNDLVPEFLPAPEDVETNCE | | |
| | | WSAFSCFQKAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_4143 | V57N, R94Q, S99A, | MRSSPGNMERIVICLMVIFLGTLVHKSSSQGQDRHM | 1.275 | 545 |
| | A112Y | IRMRQLIDIVDQLKNYVNDLNPEFLPAPEDVETNCE | | |
| | | WSAFSCFQKAQLKSANTGNNEQIINVAIKKLKRKPP | | |
| | | STNYGRRQKHRLTCPSCDSYEKKPPKEFLERFKSLL | | |
| | | QKMIHQHLSSRTHGSEDS | | |
| IL-21_4807 | D55E, N88K, | MRSSPGNMERIVICLMVIFLGTLVHKSSSQGQDRHM | 1.277 | 546 |
| | G113M, R114A, | IRMRQLIDIVDQLKNYVNELVPEFLPAPEDVETNCE | | |
| | R115E | WSAFSCFQKAQLKSAKTGNNERIINVSIKKLKRKPP | | |
| | | STNAMAEQKHRLTCPSCDSYEKKPPKEFLERFKSLL | | |
| | | QKMIHQHLSSRTHGSEDS | | |
| IL-21_2896 | R115D, T121E | MRSSPGNMERIVICLMVIFLGTLVHKSSSQGQDRHM | 1.279 | 547 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRDQKHRLECPSCDSYEKKPPKEFLERFKSLL | | |
| | | QKMIHQHLSSRTHGSEDS | | |
| IL-21_4773 | T110S, R115D, | MRSSPGNMERIVICLMVIFLGTLVHKSSSQGQDRHM | 1.282 | 548 |
| | Q116M, H118I, | IRMRQLIDIVDQLKNYVNDLVPEFLPAPEDVETNCE | | |
| | H149Y | WSAFSCFQKAQLKSANTGNNERIINVSIKKLKRKPP | | |
| | | SSNAGRDMKIRLTCPSCDSYEKKPPKEFLERFKSLL | | |
| | | QKMIYQHLSSRTHGSEDS | | |
| IL-21_2918 | N70E, N111D | MRSSPGNMERIVICLMVIFLGTLVHKSSSQGQDRHM | 1.282 | 549 |
| | | IRMRQLIDIVDQLKNYVNDLVPEFLPAPEDVETECE | | |
| | | WSAFSCFQKAQLKSANTGNNERIINVSIKKLKRKPP | | |
| | | STDAGRRQKHRLTCPSCDSYEKKPPKEFLERFKSLL | | |
| | | QKMIHQHLSSRTHGSEDS | | |
| IL-21_1468 | I45E, F60S, N97D, | MRSSPGNMERIVICLMVIFLGTLVHKSSSQGQDRHM | 1.283 | 550 |
| | K106V | IRMRQLIDEVDQLKNYVNDLVPESLPAPEDVETNCE | | |
| | | WSAFSCFQKAQLKSANTGNNERIIDVSIKKLKRVPP | | |
| | | STNAGRRQKHRLTCPSCDSYEKKPPKEFLERFKSLL | | |
| | | QKMIHQHLSSRTHGSEDS | | |
| IL-21_4062 | H35F, F60S, G113K, | MRSSPGNMERIVICLMVIFLGTLVHKSSSQGQDRFM | 1.284 | 551 |
| | R114I | IRMRQLIDIVDQLKNYVNDLVPESLPAPEDVETNCE | | |
| | | WSAFSCFQKAQLKSANTGNNERIINVSIKKLKRKPP | | |
| | | STNAKIRQKHRLTCPSCDSYEKKPPKEFLERFKSLL | | |
| | | QKMIHQHLSSRTHGSEDS | | |
| IL-21_3571 | P58V, K101E, | MRSSPGNMERIVICLMVIFLGTLVHKSSSQGQDRHM | 1.29 | 552 |
| | R114A | IRMRQLIDIVDQLKNYVNDLVVEFLPAPEDVETNCE | | |
| | | WSAFSCFQKAQLKSANTGNNERIINVSIEKLKRKPP | | |
| | | STNAGARQKHRLTCPSCDSYEKKPPKEFLERFKSLL | | |
| | | QKMIHQHLSSRTHGSEDS | | |
| IL-21_1205 | N51H, T69E, K81S | MRSSPGNMERIVICLMVIFLGTLVHKSSSQGQDRHM | 1.292 | 553 |
| | | IRMRQLIDIVDQLKHYVNDLVPEFLPAPEDVEENCE | | |
| | | WSAFSCFQSAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4196 | H35F, M36P, N88E, | MRSSPGNMERIVICLMVIFLGTLVHKSSSQGQDRFP | 1.294 | 554 |
| | V98Q | IRMRQLIDIVDQLKNYVNDLVPEFLPAPEDVETNCE | | |
| | | WSAFSCFQKAQLKSAETGNNERIINQSIKKLKRKPP | | |
| | | STNAGRRQKHRLTCPSCDSYEKKPPKEFLERFKSLL | | |
| | | QKMIHQHLSSRTHGSEDS | | |
| IL-21_4827 | D47G, S99A, | MRSSPGNMERIVICLMVIFLGTLVHKSSSQGQDRHM | 1.295 | 555 |
| | K106L, N111Q, | IRMRQLIDIVGQLKNYVNDLVPEFLPAPEDVETNCE | | |
| | R114V | WSAFSCFQKAQLKSANTGNNERIINVAIKKLKRLPP | | |
| | | STQAGVRQKHRLTCPSCDSYEKKPPKEFLERFKSLL | | |
| | | QKMIHQHLSSRTHGSEDS | | |
| IL-21_4661 | Q32F, A63T, V98I, | MRSSPGNMERIVICLMVIFLGTLVHKSSSQGFDRHM | 1.297 | 556 |
| | R114L, R115Q | IRMRQLIDIVDQLKNYVNDLVPEFLPTPEDVETNCE | | |
| | | WSAFSCFQKAQLKSANTGNNERIINISIKKLKRKPP | | |
| | | STNAGLQQKHRLTCPSCDSYEKKPPKEFLERFKSLL | | |
| | | QKMIHQHLSSRTHGSEDS | | |
| IL-21_4095 | R34L, 195L, S99L, | MRSSPGNMERIVICLMVIFLGTLVHKSSSQGQDLHM | 1.301 | 557 |
| | R114S | IRMRQLIDIVDQLKNYVNDLVPEFLPAPEDVETNCE | | |
| | | WSAFSCFQKAQLKSANTGNNERLINVLIKKLKRKPP | | |
| | | STNAGSRQKHRLTCPSCDSYEKKPPKEFLERFKSLL | | |
| | | QKMIHQHLSSRTHGSEDS | | |
| IL-21_2290 | K50T, W73E, Q83E, | MRSSPGNMERIVICLMVIFLGTLVHKSSSQGQDRHM | 1.302 | 558 |
| | N111S, A112Y | IRMRQLIDIVDQLTNYVNDLVPEFLPAPEDVETNCE | | |
| | | ESAFSCFQKAELKSANTGNNERIINVSIKKLKRKPP | | |
| | | STSYGRRQKHRLTCPSCDSYEKKPPKEFLERFKSLL | | |
| | | QKMIHQHLSSRTHGSEDS | | |
| IL-21_3446 | I37V, N111E, R114S | MRSSPGNMERIVICLMVIFLGTLVHKSSSQGQDRHM | 1.308 | 559 |
| | | VRMRQLIDIVDQLKNYVNDLVPEFLPAPEDVETNCE | | |
| | | WSAFSCFQKAQLKSANTGNNERIINVSIKKLKRKPP | | |
| | | STEAGSRQKHRLTCPSCDSYEKKPPKEFLERFKSLL | | |
| | | QKMIHQHLSSRTHGSEDS | | |
| IL-21_2807 | T69D, R115D | MRSSPGNMERIVICLMVIFLGTLVHKSSSQGQDRHM | 1.308 | 560 |
| | | IRMRQLIDIVDQLKNYVNDLVPEFLPAPEDVEDNCE | | |
| | | WSAFSCFQKAQLKSANTGNNERIINVSIKKLKRKPP | | |
| | | STNAGRDQKHRLTCPSCDSYEKKPPKEFLERFKSLL | | |
| | | QKMIHQHLSSRTHGSEDS | | |
| IL-21_2872 | R115G, Q116D | MRSSPGNMERIVICLMVIFLGTLVHKSSSQGQDRHM | 1.31 | 561 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRGDKHRLTCPSCDSYEKKPPKEFLERFKSLL | | |
| | | QKMIHQHLSSRTHGSEDS | | |
| IL-21_2292 | H35L, Q41E, I43S, | MRSSPGNMERIVICLMVIFLGTLVHKSSSQGQDRLM | 1.311 | 562 |
| | N91S, P107M | IRMRELSDIVDQLKNYVNDLVPEFLPAPEDVETNCE | | |
| | | WSAFSCFQKAQLKSANTGSNERIINVSIKKLKRKMP | | |
| | | STNAGRRQKHRLTCPSCDSYEKKPPKEFLERFKSLL | | |
| | | QKMIHQHLSSRTHGSEDS | | |
| IL-21_2902 | R40M, R114T | MRSSPGNMERIVICLMVIFLGTLVHKSSSQGQDRHM | 1.314 | 563 |
| | | IRMMQLIDIVDQLKNYVNDLVPEFLPAPEDVETNCE | | |
| | | WSAFSCFQKAQLKSANTGNNERIINVSIKKLKRKPP | | |
| | | STNAGTRQKHRLTCPSCDSYEKKPPKEFLERFKSLL | | |
| | | QKMIHQHLSSRTHGSEDS | | |
| IL-21_1236 | V53S, K102Q, T110I | MRSSPGNMERIVICLMVIFLGTLVHKSSSQGQDRHM | 1.314 | 564 |
| | | IRMRQLIDIVDQLKNYSNDLVPEFLPAPEDVETNCE | | |
| | | WSAFSCFQKAQLKSANTGNNERIINVSIKQLKRKPP | | |
| | | SINAGRRQKHRLTCPSCDSYEKKPPKEFLERFKSLL | | |
| | | QKMIHQHLSSRTHGSEDS | | |
| IL-21_4037 | R34L, R94T, N111I, | MRSSPGNMERIVICLMVIFLGTLVHKSSSQGQDLHM | 1.317 | 565 |
| | R114S | IRMRQLIDIVDQLKNYVNDLVPEFLPAPEDVETNCE | | |
| | | WSAFSCFQKAQLKSANTGNNETIINVSIKKLKRKPP | | |
| | | STIAGSRQKHRLTCPSCDSYEKKPPKEFLERFKSLL | | |
| | | QKMIHQHLSSRTHGSEDS | | |
| IL-21_3672 | A112V, G113Y, | MRSSPGNMERIVICLMVIFLGTLVHKSSSQGQDRHM | 1.319 | 566 |
| | R115E | IRMRQLIDIVDQLKNYVNDLVPEFLPAPEDVETNCE | | |
| | | WSAFSCFQKAQLKSANTGNNERIINVSIKKLKRKPP | | |
| | | STNVYREQKHRLTCPSCDSYEKKPPKEFLERFKSLL | | |
| | | QKMIHQHLSSRTHGSEDS | | |
| IL-21_2677 | M39L, N111D | MRSSPGNMERIVICLMVIFLGTLVHKSSSQGQDRHM | 1.32 | 567 |
| | | IRLRQLIDIVDQLKNYVNDLVPEFLPAPEDVETNCE | | |
| | | WSAFSCFQKAQLKSANTGNNERIINVSIKKLKRKPP | | |
| | | STDAGRRQKHRLTCPSCDSYEKKPPKEFLERFKSLL | | |
| | | QKMIHQHLSSRTHGSEDS | | |
| IL-21_1180 | N51T, V98S, R115S | MRSSPGNMERIVICLMVIFLGTLVHKSSSQGQDRHM | 1.321 | 568 |
| | | IRMRQLIDIVDQLKTYVNDLVPEFLPAPEDVETNCE | | |
| | | WSAFSCFQKAQLKSANTGNNERIINSSIKKLKRKPP | | |
| | | STNAGRSQKHRLTCPSCDSYEKKPPKEFLERFKSLL | | |
| | | QKMIHQHLSSRTHGSEDS | | |
| IL-21_4630 | E72V, V98Q, | MRSSPGNMERIVICLMVIFLGTLVHKSSSQGQDRHM | 1.321 | 569 |
| | R114W, H118Q, | IRMRQLIDIVDQLKNYVNDLVPEFLPAPEDVETNCV | | |
| | L120N | WSAFSCFQKAQLKSANTGNNERIINQSIKKLKRKPP | | |
| | | STNAGWRQKQRNTCPSCDSYEKKPPKEFLERFKSLL | | |
| | | QKMIHQHLSSRTHGSEDS | | |
| IL-21_3481 | D33V, K81T, K102Q | MRSSPGNMERIVICLMVIFLGTLVHKSSSQGQVRHM | 1.323 | 570 |
| | | IRMRQLIDIVDQLKNYVNDLVPEFLPAPEDVETNCE | | |
| | | WSAFSCFQTAQLKSANTGNNERIINVSIKQLKRKPP | | |
| | | STNAGRRQKHRLTCPSCDSYEKKPPKEFLERFKSLL | | |
| | | QKMIHQHLSSRTHGSEDS | | |
| IL-21_4202 | Q32N, A112M, | MRSSPGNMERIVICLMVIFLGTLVHKSSSQGNDRHM | 1.33 | 571 |
| | R114G, R115Y | IRMRQLIDIVDQLKNYVNDLVPEFLPAPEDVETNCE | | |
| | | WSAFSCFQKAQLKSANTGNNERIINVSIKKLKRKPP | | |
| | | STNMGGYQKHRLTCPSCDSYEKKPPKEFLERFKSLL | | |
| | | QKMIHQHLSSRTHGSEDS | | |
| IL-21_2681 | R114T, R115G | MRSSPGNMERIVICLMVIFLGTLVHKSSSQGQDRHM | 1.334 | 572 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGTGQKHRLTCPSCDSYEKKPPKEFLERFKSLL | | |
| | | QKMIHQHLSSRTHGSEDS | | |
| IL-21_3555 | N70E, K81N, G113E | MRSSPGNMERIVICLMVIFLGTLVHKSSSQGQDRHM | 1.34 | 573 |
| | | IRMRQLIDIVDQLKNYVNDLVPEFLPAPEDVETECE | | |
| | | WSAFSCFQNAQLKSANTGNNERIINVSIKKLKRKPP | | |
| | | STNAERRQKHRLTCPSCDSYEKKPPKEFLERFKSLL | | |
| | | QKMIHQHLSSRTHGSEDS | | |
| IL-21_3392 | S99L, A112G, | MRSSPGNMERIVICLMVIFLGTLVHKSSSQGQDRHM | 1.346 | 574 |
| | R119V | IRMRQLIDIVDQLKNYVNDLVPEFLPAPEDVETNCE | | |
| | | WSAFSCFQKAQLKSANTGNNERIINVLIKKLKRKPP | | |
| | | STNGGRRQKHVLTCPSCDSYEKKPPKEFLERFKSLL | | |
| | | QKMIHQHLSSRTHGSEDS | | |
| IL-21_4882 | K81T, V98L, S99T, | MRSSPGNMERIVICLMVIFLGTLVHKSSSQGQDRHM | 1.348 | 575 |
| | E138D, H149K | IRMRQLIDIVDQLKNYVNDLVPEFLPAPEDVETNCE | | |
| | | WSAFSCFQTAQLKSANTGNNERIINLTIKKLKRKPP | | |
| | | STNAGRRQKHRLTCPSCDSYEKKPPKEFLDRFKSLL | | |
| | | QKMIKQHLSSRTHGSEDS | | |
| IL-21_3008 | R115V, H118D | MRSSPGNMERIVICLMVIFLGTLVHKSSSQGQDRHM | 1.35 | 576 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRVQKDRLTCPSCDSYEKKPPKEFLERFKSLL | | |
| | | QKMIHQHLSSRTHGSEDS | | |
| IL-21_0723 | K50S, S77N, R114G | MRSSPGNMERIVICLMVIFLGTLVHKSSSQGQDRHM | 1.351 | 577 |
| | | IRMRQLIDIVDQLSNYVNDLVPEFLPAPEDVETNCE | | |
| | | WSAFNCFQKAQLKSANTGNNERIINVSIKKLKRKPP | | |
| | | STNAGGRQKHRLTCPSCDSYEKKPPKEFLERFKSLL | | |
| | | QKMIHQHLSSRTHGSEDS | | |
| IL-21_3108 | R114T, R119V | MRSSPGNMERIVICLMVIFLGTLVHKSSSQGQDRHM | 1.356 | 578 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGTRQKHVLTCPSCDSYEKKPPKEFLERFKSLL | | |
| | | QKMIHQHLSSRTHGSEDS | | |
| IL-21_3495 | P62H, K81A, R114A | MRSSPGNMERIVICLMVIFLGTLVHKSSSQGQDRHM | 1.359 | 579 |
| | | IRMRQLIDIVDQLKNYVNDLVPEFLHAPEDVETNCE | | |
| | | WSAFSCFQAAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_3685 | A87K, G113S, | MRSSPGNMERIVICLMVIFLGTLVHKSSSQGQDRHM | 1.363 | 580 |
| | R115D | IRMRQLIDIVDQLKNYVNDLVPEFLPAPEDVETNCE | | |
| | | WSAFSCFQKAQLKSKNTGNNERIINVSIKKLKRKPP | | |
| | | STNASRDQKHRLTCPSCDSYEKKPPKEFLERFKSLL | | |
| | | QKMIHQHLSSRTHGSEDS | | |
| IL-21_2853 | Q32N, R114G | MRSSPGNMERIVICLMVIFLGTLVHKSSSQGNDRHM | 1.368 | 581 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGGRQKHRLTCPSCDSYEKKPPKEFLERFKSLL | | |
| | | QKMIHQHLSSRTHGSEDS | | |
| IL-21_2761 | S99T, R115E | MRSSPGNMERIVICLMVIFLGTLVHKSSSQGQDRHM | 1.369 | 582 |
| | | IRMRQLIDIVDQLKNYVNDLVPEFLPAPEDVETNCE | | |
| | | WSAFSCFQKAQLKSANTGNNERIINVTIKKLKRKPP | | |
| | | STNAGREQKHRLTCPSCDSYEKKPPKEFLERFKSLL | | |
| | | QKMIHQHLSSRTHGSEDS | | |
| IL-21_2844 | H35E, R40D | MRSSPGNMERIVICLMVIFLGTLVHKSSSQGQDREM | 1.372 | 583 |
| | | IRMDQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2789 | R114G, H118T | MRSSPGNMERIVICLMVIFLGTLVHKSSSQGQDRHM | 1.374 | 584 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGGRQKTRLTCPSCDSYEKKPPKEFLERFKSLL | | |
| | | QKMIHQHLSSRTHGSEDS | | |
| IL-21_4687 | R34P, R40E, K50L, | MRSSPGNMERIVICLMVIFLGTLVHKSSSQGQDPHM | 1.374 | 585 |
| | N92E, R115G | IRMEQLIDIVDQLLNYVNDLVPEFLPAPEDVETNCE | | |
| | | WSAFSCFQKAQLKSANTGNEERIINVSIKKLKRKPP | | |
| | | STNAGRGQKHRLTCPSCDSYEKKPPKEFLERFKSLL | | |
| | | QKMIHQHLSSRTHGSEDS | | |
| IL-21_4280 | R40I, K81A, K106T, | MRSSPGNMERIVICLMVIFLGTLVHKSSSQGQDRHM | 1.379 | 586 |
| | N111P | IRMIQLIDIVDQLKNYVNDLVPEFLPAPEDVETNCE | | |
| | | WSAFSCFQAAQLKSANTGNNERIINVSIKKLKRTPP | | |
| | | STPAGRRQKHRLTCPSCDSYEKKPPKEFLERFKSLL | | |
| | | QKMIHQHLSSRTHGSEDS | | |
| IL-21_3440 | Q32D, D33M, H35A | MRSSPGNMERIVICLMVIFLGTLVHKSSSQGDMRAM | 1.379 | 587 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_1350 | K50S, E65K, 195D, | MRSSPGNMERIVICLMVIFLGTLVHKSSSQGQDRHM | 1.383 | 588 |
| | R114M | IRMRQLIDIVDQLSNYVNDLVPEFLPAPKDVETNCE | | |
| | | WSAFSCFQKAQLKSANTGNNERDINVSIKKLKRKPP | | |
| | | STNAGMRQKHRLTCPSCDSYEKKPPKEFLERFKSLL | | |
| | | QKMIHQHLSSRTHGSEDS | | |
| IL-21_0115 | K50S, K106E | MRSSPGNMERIVICLMVIFLGTLVHKSSSQGQDRHM | 1.385 | 589 |
| | | IRMRQLIDIVDQLSNYVNDLVPEFLPAPEDVETNCE | | |
| | | WSAFSCFQKAQLKSANTGNNERIINVSIKKLKREPP | | |
| | | STNAGRRQKHRLTCPSCDSYEKKPPKEFLERFKSLL | | |
| | | QKMIHQHLSSRTHGSEDS | | |
| IL-21_2838 | R119E, L120Q | MRSSPGNMERIVICLMVIFLGTLVHKSSSQGQDRHM | 1.386 | 590 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHEQTCPSCDSYEKKPPKEFLERFKSLL | | |
| | | QKMIHQHLSSRTHGSEDS | | |
| IL-21_4737 | A112L, R114N, | MRSSPGNMERIVICLMVIFLGTLVHKSSSQGQDRHM | 1.386 | 591 |
| | H118Q, S142N, | IRMRQLIDIVDQLKNYVNDLVPEFLPAPEDVETNCE | | |
| | Q150I | WSAFSCFQKAQLKSANTGNNERIINVSIKKLKRKPP | | |
| | | STNLGNRQKQRLTCPSCDSYEKKPPKEFLERFKNLL | | |
| | | QKMIHIHLSSRTHGSEDS | | |
| IL-21_2968 | R94T, R114Y | MRSSPGNMERIVICLMVIFLGTLVHKSSSQGQDRHM | 1.393 | 592 |
| | | IRMRQLIDIVDQLKNYVNDLVPEFLPAPEDVETNCE | | |
| | | WSAFSCFQKAQLKSANTGNNETIINVSIKKLKRKPP | | |
| | | STNAGYRQKHRLTCPSCDSYEKKPPKEFLERFKSLL | | |
| | | QKMIHQHLSSRTHGSEDS | | |
| IL-21_3459 | T110D, N111T, | MRSSPGNMERIVICLMVIFLGTLVHKSSSQGQDRHM | 1.394 | 593 |
| | H118T | IRMRQLIDIVDQLKNYVNDLVPEFLPAPEDVETNCE | | |
| | | WSAFSCFQKAQLKSANTGNNERIINVSIKKLKRKPP | | |
| | | SDTAGRRQKTRLTCPSCDSYEKKPPKEFLERFKSLL | | |
| | | QKMIHQHLSSRTHGSEDS | | |
| IL-21_4160 | V57T, S99T, A112S, | MRSSPGNMERIVICLMVIFLGTLVHKSSSQGQDRHM | 1.395 | 594 |
| | R114E | IRMRQLIDIVDQLKNYVNDLTPEFLPAPEDVETNCE | | |
| | | WSAFSCFQKAQLKSANTGNNERIINVTIKKLKRKPP | | |
| | | STNSGERQKHRLTCPSCDSYEKKPPKEFLERFKSLL | | |
| | | QKMIHQHLSSRTHGSEDS | | |
| IL-21_3524 | E68M, R114T, | MRSSPGNMERIVICLMVIFLGTLVHKSSSQGQDRHM | 1.396 | 595 |
| | R115A | IRMRQLIDIVDQLKNYVNDLVPEFLPAPEDVMTNCE | | |
| | | WSAFSCFQKAQLKSANTGNNERIINVSIKKLKRKPP | | |
| | | STNAGTAQKHRLTCPSCDSYEKKPPKEFLERFKSLL | | |
| | | QKMIHQHLSSRTHGSEDS | | |
| IL-21_3670 | V98L, G113D, | MRSSPGNMERIVICLMVIFLGTLVHKSSSQGQDRHM | 1.415 | 596 |
| | L120T | IRMRQLIDIVDQLKNYVNDLVPEFLPAPEDVETNCE | | |
| | | WSAFSCFQKAQLKSANTGNNERIINLSIKKLKRKPP | | |
| | | STNADRRQKHRTTCPSCDSYEKKPPKEFLERFKSLL | | |
| | | QKMIHQHLSSRTHGSEDS | | |
| IL-21_3457 | H35T, R40V, | MRSSPGNMERIVICLMVIFLGTLVHKSSSQGQDRTM | 1.415 | 597 |
| | G113W | IRMVQLIDIVDQLKNYVNDLVPEFLPAPEDVETNCE | | |
| | | WSAFSCFQKAQLKSANTGNNERIINVSIKKLKRKPP | | |
| | | STNAWRRQKHRLTCPSCDSYEKKPPKEFLERFKSLL | | |
| | | QKMIHQHLSSRTHGSEDS | | |
| IL-21_3069 | N111D, H118N | MRSSPGNMERIVICLMVIFLGTLVHKSSSQGQDRHM | 1.418 | 598 |
| | | IRMRQLIDIVDQLKNYVNDLVPEFLPAPEDVETNCE | | |
| | | WSAFSCFQKAQLKSANTGNNERIINVSIKKLKRKPP | | |
| | | STDAGRRQKNRLTCPSCDSYEKKPPKEFLERFKSLL | | |
| | | QKMIHQHLSSRTHGSEDS | | |
| IL-21_4150 | Q48E, V98I, N111S, | MRSSPGNMERIVICLMVIFLGTLVHKSSSQGQDRHM | 1.419 | 599 |
| | R114Q | IRMRQLIDIVDELKNYVNDLVPEFLPAPEDVETNCE | | |
| | | WSAFSCFQKAQLKSANTGNNERIINISIKKLKRKPP | | |
| | | STSAGQRQKHRLTCPSCDSYEKKPPKEFLERFKSLL | | |
| | | QKMIHQHLSSRTHGSEDS | | |
| IL-21_4132 | R34P, H35K, | MRSSPGNMERIVICLMVIFLGTLVHKSSSQGQDPKM | 1.423 | 600 |
| | K102A, R115A | IRMRQLIDIVDQLKNYVNDLVPEFLPAPEDVETNCE | | |
| | | WSAFSCFQKAQLKSANTGNNERIINVSIKALKRKPP | | |
| | | STNAGRAQKHRLTCPSCDSYEKKPPKEFLERFKSLL | | |
| | | QKMIHQHLSSRTHGSEDS | | |
| IL-21_4007 | M36F, R40K, F60L, | MRSSPGNMERIVICLMVIFLGTLVHKSSSQGQDRHE | 1.425 | 601 |
| | A112E | IRMKQLIDIVDQLKNYVNDLVPELLPAPEDVETNCE | | |
| | | WSAFSCFQKAQLKSANTGNNERIINVSIKKLKRKPP | | |
| | | STNEGRRQKHRLTCPSCDSYEKKPPKEFLERFKSLL | | |
| | | QKMIHQHLSSRTHGSEDS | | |
| IL-21_1331 | K50S, F76M, N97G, | MRSSPGNMERIVICLMVIFLGTLVHKSSSQGQDRHM | 1.428 | 602 |
| | V98E | IRMRQLIDIVDQLSNYVNDLVPEFLPAPEDVETNCE | | |
| | | WSAMSCFQKAQLKSANTGNNERIIGESIKKLKRKPP | | |
| | | STNAGRRQKHRLTCPSCDSYEKKPPKEFLERFKSLL | | |
| | | QKMIHQHLSSRTHGSEDS | | |
| IL-21_4033 | N70H, R114T, | MRSSPGNMERIVICLMVIFLGTLVHKSSSQGQDRHM | 1.431 | 603 |
| | L120G, T121D | IRMRQLIDIVDQLKNYVNDLVPEFLPAPEDVETHCE | | |
| | | WSAFSCFQKAQLKSANTGNNERIINVSIKKLKRKPP | | |
| | | STNAGTRQKHRGDCPSCDSYEKKPPKEFLERFKSLL | | |
| | | QKMIHQHLSSRTHGSEDS | | |
| IL-21_3708 | S77L, V98D, L120H | MRSSPGNMERIVICLMVIFLGTLVHKSSSQGQDRHM | 1.433 | 604 |
| | | IRMRQLIDIVDQLKNYVNDLVPEFLPAPEDVETNCE | | |
| | | WSAFLCFQKAQLKSANTGNNERIINDSIKKLKRKPP | | |
| | | STNAGRRQKHRHTCPSCDSYEKKPPKEFLERFKSLL | | |
| | | QKMIHQHLSSRTHGSEDS | | |
| IL-21_3096 | R40G, R114E | MRSSPGNMERIVICLMVIFLGTLVHKSSSQGQDRHM | 1.437 | 605 |
| | | IRMGQLIDIVDQLKNYVNDLVPEFLPAPEDVETNCE | | |
| | | WSAFSCFQKAQLKSANTGNNERIINVSIKKLKRKPP | | |
| | | STNAGERQKHRLTCPSCDSYEKKPPKEFLERFKSLL | | |
| | | QKMIHQHLSSRTHGSEDS | | |
| IL-21_3049 | V57N, R115S | MRSSPGNMERIVICLMVIFLGTLVHKSSSQGQDRHM | 1.441 | 606 |
| | | IRMRQLIDIVDQLKNYVNDLNPEFLPAPEDVETNCE | | |
| | | WSAFSCFQKAQLKSANTGNNERIINVSIKKLKRKPP | | |
| | | STNAGRSQKHRLTCPSCDSYEKKPPKEFLERFKSLL | | |
| | | QKMIHQHLSSRTHGSEDS | | |
| IL-21_3441 | S99L, N111D, | MRSSPGNMERIVICLMVIFLGTLVHKSSSQGQDRHM | 1.446 | 607 |
| | Q116S | IRMRQLIDIVDQLKNYVNDLVPEFLPAPEDVETNCE | | |
| | | WSAFSCFQKAQLKSANTGNNERIINVLIKKLKRKPP | | |
| | | STDAGRRSKHRLTCPSCDSYEKKPPKEFLERFKSLL | | |
| | | QKMIHQHLSSRTHGSEDS | | |
| IL-21_1967 | R38F, K50S, N54A, | MRSSPGNMERIVICLMVIFLGTLVHKSSSQGQDRHM | 1.45 | 608 |
| | E68D, K81A | IFMRQLIDIVDQLSNYVADLVPEFLPAPEDVDTNCE | | |
| | | WSAFSCFQAAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4250 | H35F, Y52V, V57L, | MRSSPGNMERIVICLMVIFLGTLVHKSSSQGQDRFM | 1.454 | 609 |
| | N111E | IRMRQLIDIVDQLKNVVNDLLPEFLPAPEDVETNCE | | |
| | | WSAFSCFQKAQLKSANTGNNERIINVSIKKLKRKPP | | |
| | | STEAGRRQKHRLTCPSCDSYEKKPPKEFLERFKSLL | | |
| | | QKMIHQHLSSRTHGSEDS | | |
| IL-21_0629 | N51S, F60G, K101Q | MRSSPGNMERIVICLMVIFLGTLVHKSSSQGQDRHM | 1.456 | 610 |
| | | IRMRQLIDIVDQLKSYVNDLVPEGLPAPEDVETNCE | | |
| | | WSAFSCFQKAQLKSANTGNNERIINVSIQKLKRKPP | | |
| | | STNAGRRQKHRLTCPSCDSYEKKPPKEFLERFKSLL | | |
| | | QKMIHQHLSSRTHGSEDS | | |
| IL-21_3019 | V98S, R115G | MRSSPGNMERIVICLMVIFLGTLVHKSSSQGQDRHM | 1.456 | 611 |
| | | IRMRQLIDIVDQLKNYVNDLVPEFLPAPEDVETNCE | | |
| | | WSAFSCFQKAQLKSANTGNNERIINSSIKKLKRKPP | | |
| | | STNAGRGQKHRLTCPSCDSYEKKPPKEFLERFKSLL | | |
| | | QKMIHQHLSSRTHGSEDS | | |
| IL-21_2895 | Q32N, N111E | MRSSPGNMERIVICLMVIFLGTLVHKSSSQGNDRHM | 1.456 | 612 |
| | | IRMRQLIDIVDQLKNYVNDLVPEFLPAPEDVETNCE | | |
| | | WSAFSCFQKAQLKSANTGNNERIINVSIKKLKRKPP | | |
| | | STEAGRRQKHRLTCPSCDSYEKKPPKEFLERFKSLL | | |
| | | QKMIHQHLSSRTHGSEDS | | |
| IL-21_1923 | Q48D, K50S, E65G, | MRSSPGNMERIVICLMVIFLGTLVHKSSSQGQDRHM | 1.461 | 613 |
| | V98E, P108Y | IRMRQLIDIVDDLSNYVNDLVPEFLPAPGDVETNCE | | |
| | | WSAFSCFQKAQLKSANTGNNERIINESIKKLKRKPY | | |
| | | STNAGRRQKHRLTCPSCDSYEKKPPKEFLERFKSLL | | |
| | | QKMIHQHLSSRTHGSEDS | | |
| IL-21_4804 | N54K, E72V, N91E, | MRSSPGNMERIVICLMVIFLGTLVHKSSSQGQDRHM | 1.463 | 614 |
| | R94A, G113E | IRMRQLIDIVDQLKNYVKDLVPEFLPAPEDVETNCV | | |
| | | WSAFSCFQKAQLKSANTGENEAIINVSIKKLKRKPP | | |
| | | STNAERRQKHRLTCPSCDSYEKKPPKEFLERFKSLL | | |
| | | QKMIHQHLSSRTHGSEDS | | |
| IL-21_3570 | N97S, V98L, R114V | MRSSPGNMERIVICLMVIFLGTLVHKSSSQGQDRHM | 1.464 | 615 |
| | | IRMRQLIDIVDQLKNYVNDLVPEFLPAPEDVETNCE | | |
| | | WSAFSCFQKAQLKSANTGNNERIISLSIKKLKRKPP | | |
| | | STNAGVRQKHRLTCPSCDSYEKKPPKEFLERFKSLL | | |
| | | QKMIHQHLSSRTHGSEDS | | |
| IL-21_2931 | Q116N, H118E | MRSSPGNMERIVICLMVIFLGTLVHKSSSQGQDRHM | 1.465 | 616 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRNKERLTCPSCDSYEKKPPKEFLERFKSLL | | |
| | | QKMIHQHLSSRTHGSEDS | | |
| IL-21_2353 | Q32G, H35P, M36E, | MRSSPGNMERIVICLMVIFLGTLVHKSSSQGGDRPE | 1.468 | 617 |
| | V571, F60P | IRMRQLIDIVDQLKNYVNDLIPEPLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4221 | N97T, H118E, | MRSSPGNMERIVICLMVIFLGTLVHKSSSQGQDRHM | 1.469 | 618 |
| | R119Q, T121A | IRMRQLIDIVDQLKNYVNDLVPEFLPAPEDVETNCE | | |
| | | WSAFSCFQKAQLKSANTGNNERIITVSIKKLKRKPP | | |
| | | STNAGRRQKEQLACPSCDSYEKKPPKEFLERFKSLL | | |
| | | QKMIHQHLSSRTHGSEDS | | |
| IL-21_2360 | K50L, N51H, V53R, | MRSSPGNMERIVICLMVIFLGTLVHKSSSQGQDRHM | 1.474 | 619 |
| | K101S, P107L | IRMRQLIDIVDQLLHYRNDLVPEFLPAPEDVETNCE | | |
| | | WSAFSCFQKAQLKSANTGNNERIINVSISKLKRKLP | | |
| | | STNAGRRQKHRLTCPSCDSYEKKPPKEFLERFKSLL | | |
| | | QKMIHQHLSSRTHGSEDS | | |
| IL-21_4822 | T69M, S77H, N97T, | MRSSPGNMERIVICLMVIFLGTLVHKSSSQGQDRHM | 1.479 | 620 |
| | K102Q, P107L | IRMRQLIDIVDQLKNYVNDLVPEFLPAPEDVEMNCE | | |
| | | WSAFHCFQKAQLKSANTGNNERIITVSIKQLKRKLP | | |
| | | STNAGRRQKHRLTCPSCDSYEKKPPKEFLERFKSLL | | |
| | | QKMIHQHLSSRTHGSEDS | | |
| IL-21_3671 | H35T, R40V, D44E | MRSSPGNMERIVICLMVIFLGTLVHKSSSQGQDRTM | 1.481 | 621 |
| | | IRMVQLIEIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4101 | R94Q, 195T, N111I, | MRSSPGNMERIVICLMVIFLGTLVHKSSSQGQDRHM | 1.481 | 622 |
| | H118E | IRMRQLIDIVDQLKNYVNDLVPEFLPAPEDVETNCE | | |
| | | WSAFSCFQKAQLKSANTGNNEQTINVSIKKLKRKPP | | |
| | | STIAGRRQKERLTCPSCDSYEKKPPKEFLERFKSLL | | |
| | | QKMIHQHLSSRTHGSEDS | | |
| IL-21_2743 | M39L, K81N | MRSSPGNMERIVICLMVIFLGTLVHKSSSQGQDRHM | 1.486 | 623 |
| | | IRLRQLIDIVDQLKNYVNDLVPEFLPAPEDVETNCE | | |
| | | WSAFSCFQNAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3070 | P107L, R119G | MRSSPGNMERIVICLMVIFLGTLVHKSSSQGQDRHM | 1.49 | 624 |
| | | IRMRQLIDIVDQLKNYVNDLVPEFLPAPEDVETNCE | | |
| | | WSAFSCFQKAQLKSANTGNNERIINVSIKKLKRKLP | | |
| | | STNAGRRQKHGLTCPSCDSYEKKPPKEFLERFKSLL | | |
| | | QKMIHQHLSSRTHGSEDS | | |
| IL-21_4879 | E93Q, A112Q, | MRSSPGNMERIVICLMVIFLGTLVHKSSSQGQDRHM | 1.492 | 625 |
| | R115E, R119K, | IRMRQLIDIVDQLKNYVNDLVPEFLPAPEDVETNCE | | |
| | H151T | WSAFSCFQKAQLKSANTGNNQRIINVSIKKLKRKPP | | |
| | | STNQGREQKHKLTCPSCDSYEKKPPKEFLERFKSLL | | |
| | | QKMIHQTLSSRTHGSEDS | | |
| IL-21_4754 | H35L, F60L, 195G, | MRSSPGNMERIVICLMVIFLGTLVHKSSSQGQDRLM | 1.495 | 626 |
| | G113I, R114T | IRMRQLIDIVDQLKNYVNDLVPELLPAPEDVETNCE | | |
| | | WSAFSCFQKAQLKSANTGNNERGINVSIKKLKRKPP | | |
| | | STNAITRQKHRLTCPSCDSYEKKPPKEFLERFKSLL | | |
| | | QKMIHQHLSSRTHGSEDS | | |
| IL-21_1032 | N51E, F60E, D66E | MRSSPGNMERIVICLMVIFLGTLVHKSSSQGQDRHM | 1.5 | 627 |
| | | IRMRQLIDIVDQLKEYVNDLVPEELPAPEEVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3084 | M39L, K106D | MRSSPGNMERIVICLMVIFLGTLVHKSSSQGQDRHM | 1.5 | 628 |
| | | IRLRQLIDIVDQLKNYVNDLVPEFLPAPEDVETNCE | | |
| | | WSAFSCFQKAQLKSANTGNNERIINVSIKKLKRDPP | | |
| | | STNAGRRQKHRLTCPSCDSYEKKPPKEFLERFKSLL | | |
| | | QKMIHQHLSSRTHGSEDS | | |
| IL-21_3511 | V98I, R119I, T121E | MRSSPGNMERIVICLMVIFLGTLVHKSSSQGQDRHM | 1.509 | 629 |
| | | IRMRQLIDIVDQLKNYVNDLVPEFLPAPEDVETNCE | | |
| | | WSAFSCFQKAQLKSANTGNNERIINISIKKLKRKPP | | |
| | | STNAGRRQKHILECPSCDSYEKKPPKEFLERFKSLL | | |
| | | QKMIHQHLSSRTHGSEDS | | |
| IL-21_4178 | Q32P, H35V, R94Q, | MRSSPGNMERIVICLMVIFLGTLVHKSSSQGPDRVM | 1.51 | 630 |
| | A112Q | IRMRQLIDIVDQLKNYVNDLVPEFLPAPEDVETNCE | | |
| | | WSAFSCFQKAQLKSANTGNNEQIINVSIKKLKRKPP | | |
| | | STNQGRRQKHRLTCPSCDSYEKKPPKEFLERFKSLL | | |
| | | QKMIHQHLSSRTHGSEDS | | |
| IL-21_4066 | M36L, T89S, N91T, | MRSSPGNMERIVICLMVIFLGTLVHKSSSQGQDRHL | 1.514 | 631 |
| | R115G | IRMRQLIDIVDQLKNYVNDLVPEFLPAPEDVETNCE | | |
| | | WSAFSCFQKAQLKSANSGTNERIINVSIKKLKRKPP | | |
| | | STNAGRGQKHRLTCPSCDSYEKKPPKEFLERFKSLL | | |
| | | QKMIHQHLSSRTHGSEDS | | |
| IL-21_3586 | N54E, Q83G, R114A | MRSSPGNMERIVICLMVIFLGTLVHKSSSQGQDRHM | 1.515 | 632 |
| | | IRMRQLIDIVDQLKNYVEDLVPEFLPAPEDVETNCE | | |
| | | WSAFSCFQKAGLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_2282 | N54S, F60E, N70V, | MRSSPGNMERIVICLMVIFLGTLVHKSSSQGQDRHM | 1.518 | 633 |
| | R114K, R115E | IRMRQLIDIVDQLKNYVSDLVPEELPAPEDVETVCE | | |
| | | WSAFSCFQKAQLKSANTGNNERIINVSIKKLKRKPP | | |
| | | STNAGKEQKHRLTCPSCDSYEKKPPKEFLERFKSLL | | |
| | | QKMIHQHLSSRTHGSEDS | | |
| IL-21_2800 | R114K, R115G | MRSSPGNMERIVICLMVIFLGTLVHKSSSQGQDRHM | 1.52 | 634 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGKGQKHRLTCPSCDSYEKKPPKEFLERFKSLL | | |
| | | QKMIHQHLSSRTHGSEDS | | |
| IL-21_3107 | R114T, R119L | MRSSPGNMERIVICLMVIFLGTLVHKSSSQGQDRHM | 1.524 | 635 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGTRQKHLLTCPSCDSYEKKPPKEFLERFKSLL | | |
| | | QKMIHQHLSSRTHGSEDS | | |
| IL-21_2687 | H35S, N70E | MRSSPGNMERIVICLMVIFLGTLVHKSSSQGQDRSM | 1.525 | 636 |
| | | IRMRQLIDIVDQLKNYVNDLVPEFLPAPEDVETECE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2889 | H35Q, V57E | MRSSPGNMERIVICLMVIFLGTLVHKSSSQGQDRQM | 1.527 | 637 |
| | | IRMRQLIDIVDQLKNYVNDLEPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2758 | R114T, R119T | MRSSPGNMERIVICLMVIFLGTLVHKSSSQGQDRHM | 1.529 | 638 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGTRQKHTLTCPSCDSYEKKPPKEFLERFKSLL | | |
| | | QKMIHQHLSSRTHGSEDS | | |
| IL-21_3473 | Q32D, A87S, R115G | MRSSPGNMERIVICLMVIFLGTLVHKSSSQGDDRHM | 1.534 | 639 |
| | | IRMRQLIDIVDQLKNYVNDLVPEFLPAPEDVETNCE | | |
| | | WSAFSCFQKAQLKSSNTGNNERIINVSIKKLKRKPP | | |
| | | STNAGRGQKHRLTCPSCDSYEKKPPKEFLERFKSLL | | |
| | | QKMIHQHLSSRTHGSEDS | | |
| IL-21_4779 | H35S, R38F, S86L, | MRSSPGNMERIVICLMVIFLGTLVHKSSSQGQDRSM | 1.537 | 640 |
| | N111E, A112P | IFMRQLIDIVDQLKNYVNDLVPEFLPAPEDVETNCE | | |
| | | WSAFSCFQKAQLKLANTGNNERIINVSIKKLKRKPP | | |
| | | STEPGRRQKHRLTCPSCDSYEKKPPKEFLERFKSLL | | |
| | | QKMIHQHLSSRTHGSEDS | | |
| IL-21_4118 | H35K, K81D, N97T, | MRSSPGNMERIVICLMVIFLGTLVHKSSSQGQDRKM | 1.538 | 641 |
| | N111P | IRMRQLIDIVDQLKNYVNDLVPEFLPAPEDVETNCE | | |
| | | WSAFSCFQDAQLKSANTGNNERIITVSIKKLKRKPP | | |
| | | STPAGRRQKHRLTCPSCDSYEKKPPKEFLERFKSLL | | |
| | | QKMIHQHLSSRTHGSEDS | | |
| IL-21_3351 | R34T, N111E, | MRSSPGNMERIVICLMVIFLGTLVHKSSSQGQDTHM | 1.538 | 642 |
| | R114V | IRMRQLIDIVDQLKNYVNDLVPEFLPAPEDVETNCE | | |
| | | WSAFSCFQKAQLKSANTGNNERIINVSIKKLKRKPP | | |
| | | STEAGVRQKHRLTCPSCDSYEKKPPKEFLERFKSLL | | |
| | | QKMIHQHLSSRTHGSEDS | | |
| IL-21_1586 | Q32A, H35A, N51H, | MRSSPGNMERIVICLMVIFLGTLVHKSSSQGADRAM | 1.538 | 643 |
| | V98A | IRMRQLIDIVDQLKHYVNDLVPEFLPAPEDVETNCE | | |
| | | WSAFSCFQKAQLKSANTGNNERIINASIKKLKRKPP | | |
| | | STNAGRRQKHRLTCPSCDSYEKKPPKEFLERFKSLL | | |
| | | QKMIHQHLSSRTHGSEDS | | |
| IL-21_2940 | S77T, H118D | MRSSPGNMERIVICLMVIFLGTLVHKSSSQGQDRHM | 1.547 | 644 |
| | | IRMRQLIDIVDQLKNYVNDLVPEFLPAPEDVETNCE | | |
| | | WSAFTCFQKAQLKSANTGNNERIINVSIKKLKRKPP | | |
| | | STNAGRRQKDRLTCPSCDSYEKKPPKEFLERFKSLL | | |
| | | QKMIHQHLSSRTHGSEDS | | |
| IL-21_2718 | R40E, R114Q | MRSSPGNMERIVICLMVIFLGTLVHKSSSQGQDRHM | 1.554 | 645 |
| | | IRMEQLIDIVDQLKNYVNDLVPEFLPAPEDVETNCE | | |
| | | WSAFSCFQKAQLKSANTGNNERIINVSIKKLKRKPP | | |
| | | STNAGQRQKHRLTCPSCDSYEKKPPKEFLERFKSLL | | |
| | | QKMIHQHLSSRTHGSEDS | | |
| IL-21_3577 | H35T, V98D, R105K | MRSSPGNMERIVICLMVIFLGTLVHKSSSQGQDRTM | 1.554 | 646 |
| | | IRMRQLIDIVDQLKNYVNDLVPEFLPAPEDVETNCE | | |
| | | WSAFSCFQKAQLKSANTGNNERIINDSIKKLKKKPP | | |
| | | STNAGRRQKHRLTCPSCDSYEKKPPKEFLERFKSLL | | |
| | | QKMIHQHLSSRTHGSEDS | | |
| IL-21_1540 | R40L, D44S, N97E, | MRSSPGNMERIVICLMVIFLGTLVHKSSSQGQDRHM | 1.555 | 647 |
| | R114F | IRMLQLISIVDQLKNYVNDLVPEFLPAPEDVETNCE | | |
| | | WSAFSCFQKAQLKSANTGNNERIIEVSIKKLKRKPP | | |
| | | STNAGFRQKHRLTCPSCDSYEKKPPKEFLERFKSLL | | |
| | | QKMIHQHLSSRTHGSEDS | | |
| IL-21_2723 | T69D, R114G | MRSSPGNMERIVICLMVIFLGTLVHKSSSQGQDRHM | 1.556 | 648 |
| | | IRMRQLIDIVDQLKNYVNDLVPEFLPAPEDVEDNCE | | |
| | | WSAFSCFQKAQLKSANTGNNERIINVSIKKLKRKPP | | |
| | | STNAGGRQKHRLTCPSCDSYEKKPPKEFLERFKSLL | | |
| | | QKMIHQHLSSRTHGSEDS | | |
| IL-21_4726 | K85S, S109A, | MRSSPGNMERIVICLMVIFLGTLVHKSSSQGQDRHM | 1.557 | 649 |
| | R114A, L120N, | IRMRQLIDIVDQLKNYVNDLVPEFLPAPEDVETNCE | | |
| | H149K | WSAFSCFQKAQLSSANTGNNERIINVSIKKLKRKPP | | |
| | | ATNAGARQKHRNTCPSCDSYEKKPPKEFLERFKSLL | | |
| | | QKMIKQHLSSRTHGSEDS | | |
| IL-21_2797 | H35G, R94T | MRSSPGNMERIVICLMVIFLGTLVHKSSSQGQDRGM | 1.559 | 650 |
| | | IRMRQLIDIVDQLKNYVNDLVPEFLPAPEDVETNCE | | |
| | | WSAFSCFQKAQLKSANTGNNETIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0489 | Q32P, F60E | MRSSPGNMERIVICLMVIFLGTLVHKSSSQGPDRHM | 1.56 | 651 |
| | | IRMRQLIDIVDQLKNYVNDLVPEELPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3075 | H118N, R119T | MRSSPGNMERIVICLMVIFLGTLVHKSSSQGQDRHM | 1.564 | 652 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKNTLTCPSCDSYEKKPPKEFLERFKSLL | | |
| | | QKMIHQHLSSRTHGSEDS | | |
| IL-21_4892 | H35S, M36L, P58E, | MRSSPGNMERIVICLMVIFLGTLVHKSSSQGQDRSL | 1.565 | 653 |
| | K104A, R114H | IRMRQLIDIVDQLKNYVNDLVEEFLPAPEDVETNCE | | |
| | | WSAFSCFQKAQLKSANTGNNERIINVSIKKLARKPP | | |
| | | STNAGHRQKHRLTCPSCDSYEKKPPKEFLERFKSLL | | |
| | | QKMIHQHLSSRTHGSEDS | | |
| IL-21_2274 | I37Y, N51H, D66K, | MRSSPGNMERIVICLMVIFLGTLVHKSSSQGQDRHM | 1.57 | 654 |
| | V98E, K106T | YRMRQLIDIVDQLKHYVNDLVPEFLPAPEKVETNCE | | |
| | | WSAFSCFQKAQLKSANTGNNERIINESIKKLKRTPP | | |
| | | STNAGRRQKHRLTCPSCDSYEKKPPKEFLERFKSLL | | |
| | | QKMIHQHLSSRTHGSEDS | | |
| IL-21_1392 | H35S, K50G, F60N, | MRSSPGNMERIVICLMVIFLGTLVHKSSSQGQDRSM | 1.571 | 655 |
| | L61Y | IRMRQLIDIVDQLGNYVNDLVPENYPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2782 | R114T, H118N | MRSSPGNMERIVICLMVIFLGTLVHKSSSQGQDRHM | 1.575 | 656 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGTRQKNRLTCPSCDSYEKKPPKEFLERFKSLL | | |
| | | QKMIHQHLSSRTHGSEDS | | |
| IL-21_2724 | N111S, R119E | MRSSPGNMERIVICLMVIFLGTLVHKSSSQGQDRHM | 1.576 | 657 |
| | | IRMRQLIDIVDQLKNYVNDLVPEFLPAPEDVETNCE | | |
| | | WSAFSCFQKAQLKSANTGNNERIINVSIKKLKRKPP | | |
| | | STSAGRRQKHELTCPSCDSYEKKPPKEFLERFKSLL | | |
| | | QKMIHQHLSSRTHGSEDS | | |
| IL-21_2861 | R40K, N91E | MRSSPGNMERIVICLMVIFLGTLVHKSSSQGQDRHM | 1.582 | 658 |
| | | IRMKQLIDIVDQLKNYVNDLVPEFLPAPEDVETNCE | | |
| | | WSAFSCFQKAQLKSANTGENERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4659 | H35V, K50G, T69E, | MRSSPGNMERIVICLMVIFLGTLVHKSSSQGQDRVM | 1.582 | 659 |
| | Q83T, S99G | IRMRQLIDIVDQLGNYVNDLVPEFLPAPEDVEENCE | | |
| | | WSAFSCFQKATLKSANTGNNERIINVGIKKLKRKPP | | |
| | | STNAGRRQKHRLTCPSCDSYEKKPPKEFLERFKSLL | | |
| | | QKMIHQHLSSRTHGSEDS | | |
| IL-21_3616 | N97T, R114V, | MRSSPGNMERIVICLMVIFLGTLVHKSSSQGQDRHM | 1.585 | 660 |
| | R115F | IRMRQLIDIVDQLKNYVNDLVPEFLPAPEDVETNCE | | |
| | | WSAFSCFQKAQLKSANTGNNERIITVSIKKLKRKPP | | |
| | | STNAGVFQKHRLTCPSCDSYEKKPPKEFLERFKSLL | | |
| | | QKMIHQHLSSRTHGSEDS | | |
| IL-21_3417 | K50E, N111Q, | MRSSPGNMERIVICLMVIFLGTLVHKSSSQGQDRHM | 1.592 | 661 |
| | R115G | IRMRQLIDIVDQLENYVNDLVPEFLPAPEDVETNCE | | |
| | | WSAFSCFQKAQLKSANTGNNERIINVSIKKLKRKPP | | |
| | | STQAGRGQKHRLTCPSCDSYEKKPPKEFLERFKSLL | | |
| | | QKMIHQHLSSRTHGSEDS | | |
| IL-21_3012 | S109A, R115E | MRSSPGNMERIVICLMVIFLGTLVHKSSSQGQDRHM | 1.598 | 662 |
| | | IRMRQLIDIVDQLKNYVNDLVPEFLPAPEDVETNCE | | |
| | | WSAFSCFQKAQLKSANTGNNERIINVSIKKLKRKPP | | |
| | | ATNAGREQKHRLTCPSCDSYEKKPPKEFLERFKSLL | | |
| | | QKMIHQHLSSRTHGSEDS | | |
| IL-21_2839 | R94T, S109P | MRSSPGNMERIVICLMVIFLGTLVHKSSSQGQDRHM | 1.599 | 663 |
| | | IRMRQLIDIVDQLKNYVNDLVPEFLPAPEDVETNCE | | |
| | | WSAFSCFQKAQLKSANTGNNETIINVSIKKLKRKPP | | |
| | | PTNAGRRQKHRLTCPSCDSYEKKPPKEFLERFKSLL | | |
| | | QKMIHQHLSSRTHGSEDS | | |
| IL-21_2786 | N70E, R114S | MRSSPGNMERIVICLMVIFLGTLVHKSSSQGQDRHM | 1.6 | 664 |
| | | IRMRQLIDIVDQLKNYVNDLVPEFLPAPEDVETECE | | |
| | | WSAFSCFQKAQLKSANTGNNERIINVSIKKLKRKPP | | |
| | | STNAGSRQKHRLTCPSCDSYEKKPPKEFLERFKSLL | | |
| | | QKMIHQHLSSRTHGSEDS | | |
| IL-21_3169 | D33E, H35T, M36I | MRSSPGNMERIVICLMVIFLGTLVHKSSSQGQERTI | 1.601 | 665 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3000 | S99T, R115V | MRSSPGNMERIVICLMVIFLGTLVHKSSSQGQDRHM | 1.608 | 666 |
| | | IRMRQLIDIVDQLKNYVNDLVPEFLPAPEDVETNCE | | |
| | | WSAFSCFQKAQLKSANTGNNERIINVTIKKLKRKPP | | |
| | | STNAGRVQKHRLTCPSCDSYEKKPPKEFLERFKSLL | | |
| | | QKMIHQHLSSRTHGSEDS | | |
| IL-21_2833 | H35A, T69D | MRSSPGNMERIVICLMVIFLGTLVHKSSSQGQDRAM | 1.609 | 667 |
| | | IRMRQLIDIVDQLKNYVNDLVPEFLPAPEDVEDNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4307 | R38F, N70H, S99D, | MRSSPGNMERIVICLMVIFLGTLVHKSSSQGQDRHM | 1.612 | 668 |
| | R115A | IFMRQLIDIVDQLKNYVNDLVPEFLPAPEDVETHCE | | |
| | | WSAFSCFQKAQLKSANTGNNERIINVDIKKLKRKPP | | |
| | | STNAGRAQKHRLTCPSCDSYEKKPPKEFLERFKSLL | | |
| | | QKMIHQHLSSRTHGSEDS | | |
| IL-21_3908 | I37E, K81L, R94Q, | MRSSPGNMERIVICLMVIFLGTLVHKSSSQGQDRHM | 1.614 | 669 |
| | R115P | ERMRQLIDIVDQLKNYVNDLVPEFLPAPEDVETNCE | | |
| | | WSAFSCFQLAQLKSANTGNNEQIINVSIKKLKRKPP | | |
| | | STNAGRPQKHRLTCPSCDSYEKKPPKEFLERFKSLL | | |
| | | QKMIHQHLSSRTHGSEDS | | |
| IL-21_3003 | M36T, Q83E | MRSSPGNMERIVICLMVIFLGTLVHKSSSQGQDRHT | 1.618 | 670 |
| | | IRMRQLIDIVDQLKNYVNDLVPEFLPAPEDVETNCE | | |
| | | WSAFSCFQKAELKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2856 | A112G, R114N | MRSSPGNMERIVICLMVIFLGTLVHKSSSQGQDRHM | 1.619 | 671 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNGGNRQKHRLTCPSCDSYEKKPPKEFLERFKSLL | | |
| | | QKMIHQHLSSRTHGSEDS | | |
| IL-21_3097 | R40T, R114V | MRSSPGNMERIVICLMVIFLGTLVHKSSSQGQDRHM | 1.62 | 672 |
| | | IRMTQLIDIVDQLKNYVNDLVPEFLPAPEDVETNCE | | |
| | | WSAFSCFQKAQLKSANTGNNERIINVSIKKLKRKPP | | |
| | | STNAGVRQKHRLTCPSCDSYEKKPPKEFLERFKSLL | | |
| | | QKMIHQHLSSRTHGSEDS | | |
| IL-21_4187 | D33E, H35I, L42M, | MRSSPGNMERIVICLMVIFLGTLVHKSSSQGQERIM | 1.621 | 673 |
| | E72Y | IRMRQMIDIVDQLKNYVNDLVPEFLPAPEDVETNCY | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2154 | R34D, H35S, F60T, | MRSSPGNMERIVICLMVIFLGTLVHKSSSQGQDDSM | 1.622 | 674 |
| | N88K, R94E | IRMRQLIDIVDQLKNYVNDLVPETLPAPEDVETNCE | | |
| | | WSAFSCFQKAQLKSAKTGNNEEIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3040 | H35T, S109I | MRSSPGNMERIVICLMVIFLGTLVHKSSSQGQDRTM | 1.625 | 675 |
| | | IRMRQLIDIVDQLKNYVNDLVPEFLPAPEDVETNCE | | |
| | | WSAFSCFQKAQLKSANTGNNERIINVSIKKLKRKPP | | |
| | | ITNAGRRQKHRLTCPSCDSYEKKPPKEFLERFKSLL | | |
| | | QKMIHQHLSSRTHGSEDS | | |
| IL-21_2691 | H35G, R40E | MRSSPGNMERIVICLMVIFLGTLVHKSSSQGQDRGM | 1.625 | 676 |
| | | IRMEQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3449 | R34P, S99A, A112E | MRSSPGNMERIVICLMVIFLGTLVHKSSSQGQDPHM | 1.626 | 677 |
| | | IRMRQLIDIVDQLKNYVNDLVPEFLPAPEDVETNCE | | |
| | | WSAFSCFQKAQLKSANTGNNERIINVAIKKLKRKPP | | |
| | | STNEGRRQKHRLTCPSCDSYEKKPPKEFLERFKSLL | | |
| | | QKMIHQHLSSRTHGSEDS | | |
| IL-21_4279 | Q32M, E72D, S99I, | MRSSPGNMERIVICLMVIFLGTLVHKSSSQGMDRHM | 1.631 | 678 |
| | R115M | IRMRQLIDIVDQLKNYVNDLVPEFLPAPEDVETNCD | | |
| | | WSAFSCFQKAQLKSANTGNNERIINVIIKKLKRKPP | | |
| | | STNAGRMQKHRLTCPSCDSYEKKPPKEFLERFKSLL | | |
| | | QKMIHQHLSSRTHGSEDS | | |
| IL-21_1382 | D33H, K50S, Q83E, | MRSSPGNMERIVICLMVIFLGTLVHKSSSQGQHRHM | 1.632 | 679 |
| | R114N | IRMRQLIDIVDQLSNYVNDLVPEFLPAPEDVETNCE | | |
| | | WSAFSCFQKAELKSANTGNNERIINVSIKKLKRKPP | | |
| | | STNAGNRQKHRLTCPSCDSYEKKPPKEFLERFKSLL | | |
| | | QKMIHQHLSSRTHGSEDS | | |
| IL-21_3072 | H35G, V98Q | MRSSPGNMERIVICLMVIFLGTLVHKSSSQGQDRGM | 1.637 | 680 |
| | | IRMRQLIDIVDQLKNYVNDLVPEFLPAPEDVETNCE | | |
| | | WSAFSCFQKAQLKSANTGNNERIINQSIKKLKRKPP | | |
| | | STNAGRRQKHRLTCPSCDSYEKKPPKEFLERFKSLL | | |
| | | QKMIHQHLSSRTHGSEDS | | |
| IL-21_3492 | N70E, R94T, S142N | MRSSPGNMERIVICLMVIFLGTLVHKSSSQGQDRHM | 1.638 | 681 |
| | | IRMRQLIDIVDQLKNYVNDLVPEFLPAPEDVETECE | | |
| | | WSAFSCFQKAQLKSANTGNNETIINVSIKKLKRKPP | | |
| | | STNAGRRQKHRLTCPSCDSYEKKPPKEFLERFKNLL | | |
| | | QKMIHQHLSSRTHGSEDS | | |
| IL-21_4800 | R94E, N97K, P107T, | MRSSPGNMERIVICLMVIFLGTLVHKSSSQGQDRHM | 1.638 | 682 |
| | R119I, H151R | IRMRQLIDIVDQLKNYVNDLVPEFLPAPEDVETNCE | | |
| | | WSAFSCFQKAQLKSANTGNNEEIIKVSIKKLKRKTP | | |
| | | STNAGRRQKHILTCPSCDSYEKKPPKEFLERFKSLL | | |
| | | QKMIHQRLSSRTHGSEDS | | |
| IL-21_4107 | R40K, N91K, | MRSSPGNMERIVICLMVIFLGTLVHKSSSQGQDRHM | 1.646 | 683 |
| | K106T, R114G | IRMKQLIDIVDQLKNYVNDLVPEFLPAPEDVETNCE | | |
| | | WSAFSCFQKAQLKSANTGKNERIINVSIKKLKRTPP | | |
| | | STNAGGRQKHRLTCPSCDSYEKKPPKEFLERFKSLL | | |
| | | QKMIHQHLSSRTHGSEDS | | |
| IL-21_0982 | N51H, V57D, V98S | MRSSPGNMERIVICLMVIFLGTLVHKSSSQGQDRHM | 1.647 | 684 |
| | | IRMRQLIDIVDQLKHYVNDLDPEFLPAPEDVETNCE | | |
| | | WSAFSCFQKAQLKSANTGNNERIINSSIKKLKRKPP | | |
| | | STNAGRRQKHRLTCPSCDSYEKKPPKEFLERFKSLL | | |
| | | QKMIHQHLSSRTHGSEDS | | |
| IL-21_3604 | R115G, L120S, | MRSSPGNMERIVICLMVIFLGTLVHKSSSQGQDRHM | 1.648 | 685 |
| | Q150H | IRMRQLIDIVDQLKNYVNDLVPEFLPAPEDVETNCE | | |
| | | WSAFSCFQKAQLKSANTGNNERIINVSIKKLKRKPP | | |
| | | STNAGRGQKHRSTCPSCDSYEKKPPKEFLERFKSLL | | |
| | | QKMIHHHLSSRTHGSEDS | | |
| IL-21_3025 | N51E, R114A | MRSSPGNMERIVICLMVIFLGTLVHKSSSQGQDRHM | 1.648 | 686 |
| | | IRMRQLIDIVDQLKEYVNDLVPEFLPAPEDVETNCE | | |
| | | WSAFSCFQKAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_4162 | E68G, T69E, S99A, | MRSSPGNMERIVICLMVIFLGTLVHKSSSQGQDRHM | 1.648 | 687 |
| | A112V | IRMRQLIDIVDQLKNYVNDLVPEFLPAPEDVGENCE | | |
| | | WSAFSCFQKAQLKSANTGNNERIINVAIKKLKRKPP | | |
| | | STNVGRRQKHRLTCPSCDSYEKKPPKEFLERFKSLL | | |
| | | QKMIHQHLSSRTHGSEDS | | |
| IL-21_3076 | R114L, R119T | MRSSPGNMERIVICLMVIFLGTLVHKSSSQGQDRHM | 1.65 | 688 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGLRQKHTLTCPSCDSYEKKPPKEFLERFKSLL | | |
| | | QKMIHQHLSSRTHGSEDS | | |
| IL-21_4873 | D33V, H35P, V53I, | MRSSPGNMERIVICLMVIFLGTLVHKSSSQGQVRPM | 1.651 | 689 |
| | K101E, R114A | IRMRQLIDIVDQLKNYINDLVPEFLPAPEDVETNCE | | |
| | | WSAFSCFQKAQLKSANTGNNERIINVSIEKLKRKPP | | |
| | | STNAGARQKHRLTCPSCDSYEKKPPKEFLERFKSLL | | |
| | | QKMIHQHLSSRTHGSEDS | | |
| IL-21_3978 | N70G, Q116K, | MRSSPGNMERIVICLMVIFLGTLVHKSSSQGQDRHM | 1.652 | 690 |
| | R119E, L120Y | IRMRQLIDIVDQLKNYVNDLVPEFLPAPEDVETGCE | | |
| | | WSAFSCFQKAQLKSANTGNNERIINVSIKKLKRKPP | | |
| | | STNAGRRKKHEYTCPSCDSYEKKPPKEFLERFKSLL | | |
| | | QKMIHQHLSSRTHGSEDS | | |
| IL-21_4811 | D33N, M36T, V57G, | MRSSPGNMERIVICLMVIFLGTLVHKSSSQGQNRHT | 1.658 | 691 |
| | K101E, A112L | IRMRQLIDIVDQLKNYVNDLGPEFLPAPEDVETNCE | | |
| | | WSAFSCFQKAQLKSANTGNNERIINVSIEKLKRKPP | | |
| | | STNLGRRQKHRLTCPSCDSYEKKPPKEFLERFKSLL | | |
| | | QKMIHQHLSSRTHGSEDS | | |
| IL-21_1451 | Y52S, E65Q, N97E, | MRSSPGNMERIVICLMVIFLGTLVHKSSSQGQDRHM | 1.661 | 692 |
| | K102Q | IRMRQLIDIVDQLKNSVNDLVPEFLPAPQDVETNCE | | |
| | | WSAFSCFQKAQLKSANTGNNERIIEVSIKQLKRKPP | | |
| | | STNAGRRQKHRLTCPSCDSYEKKPPKEFLERFKSLL | | |
| | | QKMIHQHLSSRTHGSEDS | | |
| IL-21_3360 | N70E, N91E, R119K | MRSSPGNMERIVICLMVIFLGTLVHKSSSQGQDRHM | 1.662 | 693 |
| | | IRMRQLIDIVDQLKNYVNDLVPEFLPAPEDVETECE | | |
| | | WSAFSCFQKAQLKSANTGENERIINVSIKKLKRKPP | | |
| | | STNAGRRQKHKLTCPSCDSYEKKPPKEFLERFKSLL | | |
| | | QKMIHQHLSSRTHGSEDS | | |
| IL-21_0160 | K50S, P107I | MRSSPGNMERIVICLMVIFLGTLVHKSSSQGQDRHM | 1.665 | 694 |
| | | IRMRQLIDIVDQLSNYVNDLVPEFLPAPEDVETNCE | | |
| | | WSAFSCFQKAQLKSANTGNNERIINVSIKKLKRKIP | | |
| | | STNAGRRQKHRLTCPSCDSYEKKPPKEFLERFKSLL | | |
| | | QKMIHQHLSSRTHGSEDS | | |
| IL-21_2901 | D33N, R115E | MRSSPGNMERIVICLMVIFLGTLVHKSSSQGQNRHM | 1.667 | 695 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGREQKHRLTCPSCDSYEKKPPKEFLERFKSLL | | |
| | | QKMIHQHLSSRTHGSEDS | | |
| IL-21_3585 | Y52M, S99A, N111T | MRSSPGNMERIVICLMVIFLGTLVHKSSSQGQDRHM | 1.668 | 696 |
| | | IRMRQLIDIVDQLKNMVNDLVPEFLPAPEDVETNCE | | |
| | | WSAFSCFQKAQLKSANTGNNERIINVAIKKLKRKPP | | |
| | | STTAGRRQKHRLTCPSCDSYEKKPPKEFLERFKSLL | | |
| | | QKMIHQHLSSRTHGSEDS | | |
| IL-21_3410 | P107L, A112L, | MRSSPGNMERIVICLMVIFLGTLVHKSSSQGQDRHM | 1.672 | 697 |
| | R119T | IRMRQLIDIVDQLKNYVNDLVPEFLPAPEDVETNCE | | |
| | | WSAFSCFQKAQLKSANTGNNERIINVSIKKLKRKLP | | |
| | | STNLGRRQKHTLTCPSCDSYEKKPPKEFLERFKSLL | | |
| | | QKMIHQHLSSRTHGSEDS | | |
| IL-21_2819 | F60L, N111E | MRSSPGNMERIVICLMVIFLGTLVHKSSSQGQDRHM | 1.674 | 698 |
| | | IRMRQLIDIVDQLKNYVNDLVPELLPAPEDVETNCE | | |
| | | WSAFSCFQKAQLKSANTGNNERIINVSIKKLKRKPP | | |
| | | STEAGRRQKHRLTCPSCDSYEKKPPKEFLERFKSLL | | |
| | | QKMIHQHLSSRTHGSEDS | | |
| IL-21_1154 | H35V, K50G, | MRSSPGNMERIVICLMVIFLGTLVHKSSSQGQDRVM | 1.674 | 699 |
| | A112Q | IRMRQLIDIVDQLGNYVNDLVPEFLPAPEDVETNCE | | |
| | | WSAFSCFQKAQLKSANTGNNERIINVSIKKLKRKPP | | |
| | | STNQGRRQKHRLTCPSCDSYEKKPPKEFLERFKSLL | | |
| | | QKMIHQHLSSRTHGSEDS | | |
| IL-21_2674 | H35T, M36A | MRSSPGNMERIVICLMVIFLGTLVHKSSSQGQDRTA | 1.676 | 700 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2890 | H35L, S99T | MRSSPGNMERIVICLMVIFLGTLVHKSSSQGQDRLM | 1.676 | 701 |
| | | IRMRQLIDIVDQLKNYVNDLVPEFLPAPEDVETNCE | | |
| | | WSAFSCFQKAQLKSANTGNNERIINVTIKKLKRKPP | | |
| | | STNAGRRQKHRLTCPSCDSYEKKPPKEFLERFKSLL | | |
| | | QKMIHQHLSSRTHGSEDS | | |
| IL-21_2238 | Q48S, K50L, P58H, | MRSSPGNMERIVICLMVIFLGTLVHKSSSQGQDRHM | 1.684 | 702 |
| | K81N, 195N | IRMRQLIDIVDSLLNYVNDLVHEFLPAPEDVETNCE | | |
| | | WSAFSCFQNAQLKSANTGNNERNINVSIKKLKRKPP | | |
| | | STNAGRRQKHRLTCPSCDSYEKKPPKEFLERFKSLL | | |
| | | QKMIHQHLSSRTHGSEDS | | |
| IL-21_2720 | N111S, R115D | MRSSPGNMERIVICLMVIFLGTLVHKSSSQGQDRHM | 1.688 | 703 |
| | | IRMRQLIDIVDQLKNYVNDLVPEFLPAPEDVETNCE | | |
| | | WSAFSCFQKAQLKSANTGNNERIINVSIKKLKRKPP | | |
| | | STSAGRDQKHRLTCPSCDSYEKKPPKEFLERFKSLL | | |
| | | QKMIHQHLSSRTHGSEDS | | |
| IL-21_3102 | R94Q, L120S | MRSSPGNMERIVICLMVIFLGTLVHKSSSQGQDRHM | 1.692 | 704 |
| | | IRMRQLIDIVDQLKNYVNDLVPEFLPAPEDVETNCE | | |
| | | WSAFSCFQKAQLKSANTGNNEQIINVSIKKLKRKPP | | |
| | | STNAGRRQKHRSTCPSCDSYEKKPPKEFLERFKSLL | | |
| | | QKMIHQHLSSRTHGSEDS | | |
| IL-21_1988 | L42M, K50S, S74P, | MRSSPGNMERIVICLMVIFLGTLVHKSSSQGQDRHM | 1.698 | 705 |
| | N88I, R114E | IRMRQMIDIVDQLSNYVNDLVPEFLPAPEDVETNCE | | |
| | | WPAFSCFQKAQLKSAITGNNERIINVSIKKLKRKPP | | |
| | | STNAGERQKHRLTCPSCDSYEKKPPKEFLERFKSLL | | |
| | | QKMIHQHLSSRTHGSEDS | | |
| IL-21_3499 | P108D, N111D, | MRSSPGNMERIVICLMVIFLGTLVHKSSSQGQDRHM | 1.699 | 706 |
| | H118Q | IRMRQLIDIVDQLKNYVNDLVPEFLPAPEDVETNCE | | |
| | | WSAFSCFQKAQLKSANTGNNERIINVSIKKLKRKPD | | |
| | | STDAGRRQKQRLTCPSCDSYEKKPPKEFLERFKSLL | | |
| | | QKMIHQHLSSRTHGSEDS | | |
| IL-21_0824 | D44S, N51V, K101E | MRSSPGNMERIVICLMVIFLGTLVHKSSSQGQDRHM | 1.7 | 707 |
| | | IRMRQLISIVDQLKVYVNDLVPEFLPAPEDVETNCE | | |
| | | WSAFSCFQKAQLKSANTGNNERIINVSIEKLKRKPP | | |
| | | STNAGRRQKHRLTCPSCDSYEKKPPKEFLERFKSLL | | |
| | | QKMIHQHLSSRTHGSEDS | | |
| IL-21_0916 | N51E, N54S, R94N | MRSSPGNMERIVICLMVIFLGTLVHKSSSQGQDRHM | 1.7 | 708 |
| | | IRMRQLIDIVDQLKEYVSDLVPEFLPAPEDVETNCE | | |
| | | WSAFSCFQKAQLKSANTGNNENIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0987 | N54E, F60E, N111A | MRSSPGNMERIVICLMVIFLGTLVHKSSSQGQDRHM | 1.701 | 709 |
| | | IRMRQLIDIVDQLKNYVEDLVPEELPAPEDVETNCE | | |
| | | WSAFSCFQKAQLKSANTGNNERIINVSIKKLKRKPP | | |
| | | STAAGRRQKHRLTCPSCDSYEKKPPKEFLERFKSLL | | |
| | | QKMIHQHLSSRTHGSEDS | | |
| IL-21_3021 | R114D, T121K | MRSSPGNMERIVICLMVIFLGTLVHKSSSQGQDRHM | 1.706 | 710 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGDRQKHRLKCPSCDSYEKKPPKEFLERFKSLL | | |
| | | QKMIHQHLSSRTHGSEDS | | |
| IL-21_2917 | H35L, R94K | MRSSPGNMERIVICLMVIFLGTLVHKSSSQGQDRLM | 1.706 | 711 |
| | | IRMRQLIDIVDQLKNYVNDLVPEFLPAPEDVETNCE | | |
| | | WSAFSCFQKAQLKSANTGNNEKIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3412 | R40E, I43V, G113D | MRSSPGNMERIVICLMVIFLGTLVHKSSSQGQDRHM | 1.708 | 712 |
| | | IRMEQLVDIVDQLKNYVNDLVPEFLPAPEDVETNCE | | |
| | | WSAFSCFQKAQLKSANTGNNERIINVSIKKLKRKPP | | |
| | | STNADRRQKHRLTCPSCDSYEKKPPKEFLERFKSLL | | |
| | | QKMIHQHLSSRTHGSEDS | | |
| IL-21_4136 | H35F, V57Q, T69Q, | MRSSPGNMERIVICLMVIFLGTLVHKSSSQGQDRFM | 1.712 | 713 |
| | N111P | IRMRQLIDIVDQLKNYVNDLQPEFLPAPEDVEQNCE | | |
| | | WSAFSCFQKAQLKSANTGNNERIINVSIKKLKRKPP | | |
| | | STPAGRRQKHRLTCPSCDSYEKKPPKEFLERFKSLL | | |
| | | QKMIHQHLSSRTHGSEDS | | |
| IL-21_3472 | H35N, E68K, R115Q | MRSSPGNMERIVICLMVIFLGTLVHKSSSQGQDRNM | 1.714 | 714 |
| | | IRMRQLIDIVDQLKNYVNDLVPEFLPAPEDVKTNCE | | |
| | | WSAFSCFQKAQLKSANTGNNERIINVSIKKLKRKPP | | |
| | | STNAGRQQKHRLTCPSCDSYEKKPPKEFLERFKSLL | | |
| | | QKMIHQHLSSRTHGSEDS | | |
| IL-21_1193 | I45T, E65V, R114T | MRSSPGNMERIVICLMVIFLGTLVHKSSSQGQDRHM | 1.715 | 715 |
| | | IRMRQLIDTVDQLKNYVNDLVPEFLPAPVDVETNCE | | |
| | | WSAFSCFQKAQLKSANTGNNERIINVSIKKLKRKPP | | |
| | | STNAGTRQKHRLTCPSCDSYEKKPPKEFLERFKSLL | | |
| | | QKMIHQHLSSRTHGSEDS | | |
| IL-21_3934 | Q32F, D33G, S99L, | MRSSPGNMERIVICLMVIFLGTLVHKSSSQGFGRHM | 1.715 | 716 |
| | R115A | IRMRQLIDIVDQLKNYVNDLVPEFLPAPEDVETNCE | | |
| | | WSAFSCFQKAQLKSANTGNNERIINVLIKKLKRKPP | | |
| | | STNAGRAQKHRLTCPSCDSYEKKPPKEFLERFKSLL | | |
| | | QKMIHQHLSSRTHGSEDS | | |
| IL-21_0371 | N51H, R114L | MRSSPGNMERIVICLMVIFLGTLVHKSSSQGQDRHM | 1.721 | 717 |
| | | IRMRQLIDIVDQLKHYVNDLVPEFLPAPEDVETNCE | | |
| | | WSAFSCFQKAQLKSANTGNNERIINVSIKKLKRKPP | | |
| | | STNAGLRQKHRLTCPSCDSYEKKPPKEFLERFKSLL | | |
| | | QKMIHQHLSSRTHGSEDS | | |
| IL-21_2974 | F76L, H118E | MRSSPGNMERIVICLMVIFLGTLVHKSSSQGQDRHM | 1.724 | 718 |
| | | IRMRQLIDIVDQLKNYVNDLVPEFLPAPEDVETNCE | | |
| | | WSALSCFQKAQLKSANTGNNERIINVSIKKLKRKPP | | |
| | | STNAGRRQKERLTCPSCDSYEKKPPKEFLERFKSLL | | |
| | | QKMIHQHLSSRTHGSEDS | | |
| IL-21_3357 | K104S, G113K, | MRSSPGNMERIVICLMVIFLGTLVHKSSSQGQDRHM | 1.725 | 719 |
| | R114A | IRMRQLIDIVDQLKNYVNDLVPEFLPAPEDVETNCE | | |
| | | WSAFSCFQKAQLKSANTGNNERIINVSIKKLSRKPP | | |
| | | STNAKARQKHRLTCPSCDSYEKKPPKEFLERFKSLL | | |
| | | QKMIHQHLSSRTHGSEDS | | |
| IL-21_0045 | N51S, R114I | MRSSPGNMERIVICLMVIFLGTLVHKSSSQGQDRHM | 1.725 | 720 |
| | | IRMRQLIDIVDQLKSYVNDLVPEFLPAPEDVETNCE | | |
| | | WSAFSCFQKAQLKSANTGNNERIINVSIKKLKRKPP | | |
| | | STNAGIRQKHRLTCPSCDSYEKKPPKEFLERFKSLL | | |
| | | QKMIHQHLSSRTHGSEDS | | |
| IL-21_2874 | R114G, R115A | MRSSPGNMERIVICLMVIFLGTLVHKSSSQGQDRHM | 1.733 | 721 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGGAQKHRLTCPSCDSYEKKPPKEFLERFKSLL | | |
| | | QKMIHQHLSSRTHGSEDS | | |
| IL-21_3010 | N111Y, H118D | MRSSPGNMERIVICLMVIFLGTLVHKSSSQGQDRHM | 1.737 | 722 |
| | | IRMRQLIDIVDQLKNYVNDLVPEFLPAPEDVETNCE | | |
| | | WSAFSCFQKAQLKSANTGNNERIINVSIKKLKRKPP | | |
| | | STYAGRRQKDRLTCPSCDSYEKKPPKEFLERFKSLL | | |
| | | QKMIHQHLSSRTHGSEDS | | |
| IL-21_2825 | Q32D, P107T | MRSSPGNMERIVICLMVIFLGTLVHKSSSQGDDRHM | 1.738 | 723 |
| | | IRMRQLIDIVDQLKNYVNDLVPEFLPAPEDVETNCE | | |
| | | WSAFSCFQKAQLKSANTGNNERIINVSIKKLKRKTP | | |
| | | STNAGRRQKHRLTCPSCDSYEKKPPKEFLERFKSLL | | |
| | | QKMIHQHLSSRTHGSEDS | | |
| IL-21_4151 | P107T, N111I, | MRSSPGNMERIVICLMVIFLGTLVHKSSSQGQDRHM | 1.74 | 724 |
| | R115A, H149N | IRMRQLIDIVDQLKNYVNDLVPEFLPAPEDVETNCE | | |
| | | WSAFSCFQKAQLKSANTGNNERIINVSIKKLKRKTP | | |
| | | STIAGRAQKHRLTCPSCDSYEKKPPKEFLERFKSLL | | |
| | | QKMINQHLSSRTHGSEDS | | |
| IL-21_4708 | N111L, R114T, | MRSSPGNMERIVICLMVIFLGTLVHKSSSQGQDRHM | 1.74 | 725 |
| | L120V, T121N, | IRMRQLIDIVDQLKNYVNDLVPEFLPAPEDVETNCE | | |
| | L152Y | WSAFSCFQKAQLKSANTGNNERIINVSIKKLKRKPP | | |
| | | STLAGTRQKHRVNCPSCDSYEKKPPKEFLERFKSLL | | |
| | | QKMIHQHYSSRTHGSEDS | | |
| IL-21_3547 | W73S, N97E, P107L | MRSSPGNMERIVICLMVIFLGTLVHKSSSQGQDRHM | 1.743 | 726 |
| | | IRMRQLIDIVDQLKNYVNDLVPEFLPAPEDVETNCE | | |
| | | SSAFSCFQKAQLKSANTGNNERIIEVSIKKLKRKLP | | |
| | | STNAGRRQKHRLTCPSCDSYEKKPPKEFLERFKSLL | | |
| | | QKMIHQHLSSRTHGSEDS | | |
| IL-21_3649 | D33S, A112Y, | MRSSPGNMERIVICLMVIFLGTLVHKSSSQGQSRHM | 1.748 | 727 |
| | R115E | IRMRQLIDIVDQLKNYVNDLVPEFLPAPEDVETNCE | | |
| | | WSAFSCFQKAQLKSANTGNNERIINVSIKKLKRKPP | | |
| | | STNYGREQKHRLTCPSCDSYEKKPPKEFLERFKSLL | | |
| | | QKMIHQHLSSRTHGSEDS | | |
| IL-21_4798 | H35S, I43A, V98G, | MRSSPGNMERIVICLMVIFLGTLVHKSSSQGQDRSM | 1.75 | 728 |
| | S991, N111A | IRMRQLADIVDQLKNYVNDLVPEFLPAPEDVETNCE | | |
| | | WSAFSCFQKAQLKSANTGNNERIINGIIKKLKRKPP | | |
| | | STAAGRRQKHRLTCPSCDSYEKKPPKEFLERFKSLL | | |
| | | QKMIHQHLSSRTHGSEDS | | |
| IL-21_3372 | E65Q, G113S, | MRSSPGNMERIVICLMVIFLGTLVHKSSSQGQDRHM | 1.75 | 729 |
| | R114A | IRMRQLIDIVDQLKNYVNDLVPEFLPAPQDVETNCE | | |
| | | WSAFSCFQKAQLKSANTGNNERIINVSIKKLKRKPP | | |
| | | STNASARQKHRLTCPSCDSYEKKPPKEFLERFKSLL | | |
| | | QKMIHQHLSSRTHGSEDS | | |
| IL-21_2993 | R115G, R119H | MRSSPGNMERIVICLMVIFLGTLVHKSSSQGQDRHM | 1.753 | 730 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRGQKHHLTCPSCDSYEKKPPKEFLERFKSLL | | |
| | | QKMIHQHLSSRTHGSEDS | | |
| IL-21_3022 | W73D, R115E | MRSSPGNMERIVICLMVIFLGTLVHKSSSQGQDRHM | 1.754 | 731 |
| | | IRMRQLIDIVDQLKNYVNDLVPEFLPAPEDVETNCE | | |
| | | DSAFSCFQKAQLKSANTGNNERIINVSIKKLKRKPP | | |
| | | STNAGREQKHRLTCPSCDSYEKKPPKEFLERFKSLL | | |
| | | QKMIHQHLSSRTHGSEDS | | |
| IL-21_1322 | R40K, Q41S, F60D, | MRSSPGNMERIVICLMVIFLGTLVHKSSSQGQDRHM | 1.754 | 732 |
| | P107D | IRMKSLIDIVDQLKNYVNDLVPEDLPAPEDVETNCE | | |
| | | WSAFSCFQKAQLKSANTGNNERIINVSIKKLKRKDP | | |
| | | STNAGRRQKHRLTCPSCDSYEKKPPKEFLERFKSLL | | |
| | | QKMIHQHLSSRTHGSEDS | | |
| IL-21_3563 | Q32E, N70H, P107M | MRSSPGNMERIVICLMVIFLGTLVHKSSSQGEDRHM | 1.755 | 733 |
| | | IRMRQLIDIVDQLKNYVNDLVPEFLPAPEDVETHCE | | |
| | | WSAFSCFQKAQLKSANTGNNERIINVSIKKLKRKMP | | |
| | | STNAGRRQKHRLTCPSCDSYEKKPPKEFLERFKSLL | | |
| | | QKMIHQHLSSRTHGSEDS | | |
| IL-21_0317 | I45T, R115N | MRSSPGNMERIVICLMVIFLGTLVHKSSSQGQDRHM | 1.756 | 734 |
| | | IRMRQLIDTVDQLKNYVNDLVPEFLPAPEDVETNCE | | |
| | | WSAFSCFQKAQLKSANTGNNERIINVSIKKLKRKPP | | |
| | | STNAGRNQKHRLTCPSCDSYEKKPPKEFLERFKSLL | | |
| | | QKMIHQHLSSRTHGSEDS | | |
| IL-21_4234 | N91S, S109P, | MRSSPGNMERIVICLMVIFLGTLVHKSSSQGQDRHM | 1.756 | 735 |
| | R114P, T121S | IRMRQLIDIVDQLKNYVNDLVPEFLPAPEDVETNCE | | |
| | | WSAFSCFQKAQLKSANTGSNERIINVSIKKLKRKPP | | |
| | | PTNAGPRQKHRLSCPSCDSYEKKPPKEFLERFKSLL | | |
| | | QKMIHQHLSSRTHGSEDS | | |
| IL-21_3385 | N70E, R94A, G113K | MRSSPGNMERIVICLMVIFLGTLVHKSSSQGQDRHM | 1.76 | 736 |
| | | IRMRQLIDIVDQLKNYVNDLVPEFLPAPEDVETECE | | |
| | | WSAFSCFQKAQLKSANTGNNEAIINVSIKKLKRKPP | | |
| | | STNAKRRQKHRLTCPSCDSYEKKPPKEFLERFKSLL | | |
| | | QKMIHQHLSSRTHGSEDS | | |
| IL-21_0287 | D44H, R94D | MRSSPGNMERIVICLMVIFLGTLVHKSSSQGQDRHM | 1.768 | 737 |
| | | IRMRQLIHIVDQLKNYVNDLVPEFLPAPEDVETNCE | | |
| | | WSAFSCFQKAQLKSANTGNNEDIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0711 | H35F, N51S, D66S | MRSSPGNMERIVICLMVIFLGTLVHKSSSQGQDRFM | 1.772 | 738 |
| | | IRMRQLIDIVDQLKSYVNDLVPEFLPAPESVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3090 | R115G, L152Q | MRSSPGNMERIVICLMVIFLGTLVHKSSSQGQDRHM | 1.774 | 739 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRGQKHRLTCPSCDSYEKKPPKEFLERFKSLL | | |
| | | QKMIHQHQSSRTHGSEDS | | |
| IL-21_2946 | N111K, R115D | MRSSPGNMERIVICLMVIFLGTLVHKSSSQGQDRHM | 1.776 | 740 |
| | | IRMRQLIDIVDQLKNYVNDLVPEFLPAPEDVETNCE | | |
| | | WSAFSCFQKAQLKSANTGNNERIINVSIKKLKRKPP | | |
| | | STKAGRDQKHRLTCPSCDSYEKKPPKEFLERFKSLL | | |
| | | QKMIHQHLSSRTHGSEDS | | |
| IL-21_2980 | Q32L, V98Q | MRSSPGNMERIVICLMVIFLGTLVHKSSSQGLDRHM | 1.78 | 741 |
| | | IRMRQLIDIVDQLKNYVNDLVPEFLPAPEDVETNCE | | |
| | | WSAFSCFQKAQLKSANTGNNERIINQSIKKLKRKPP | | |
| | | STNAGRRQKHRLTCPSCDSYEKKPPKEFLERFKSLL | | |
| | | QKMIHQHLSSRTHGSEDS | | |
| IL-21_3605 | V98I, S109A, G113E | MRSSPGNMERIVICLMVIFLGTLVHKSSSQGQDRHM | 1.782 | 742 |
| | | IRMRQLIDIVDQLKNYVNDLVPEFLPAPEDVETNCE | | |
| | | WSAFSCFQKAQLKSANTGNNERIINISIKKLKRKPP | | |
| | | ATNAERRQKHRLTCPSCDSYEKKPPKEFLERFKSLL | | |
| | | QKMIHQHLSSRTHGSEDS | | |
| IL-21_4005 | Q32L, H35F, T69R, | MRSSPGNMERIVICLMVIFLGTLVHKSSSQGLDREM | 1.783 | 743 |
| | R94A | IRMRQLIDIVDQLKNYVNDLVPEFLPAPEDVERNCE | | |
| | | WSAFSCFQKAQLKSANTGNNEAIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0681 | Q32D, N51S, K106T | MRSSPGNMERIVICLMVIFLGTLVHKSSSQGDDRHM | 1.784 | 744 |
| | | IRMRQLIDIVDQLKSYVNDLVPEFLPAPEDVETNCE | | |
| | | WSAFSCFQKAQLKSANTGNNERIINVSIKKLKRTPP | | |
| | | STNAGRRQKHRLTCPSCDSYEKKPPKEFLERFKSLL | | |
| | | QKMIHQHLSSRTHGSEDS | | |
| IL-21_2740 | V57D, G113D | MRSSPGNMERIVICLMVIFLGTLVHKSSSQGQDRHM | 1.784 | 745 |
| | | IRMRQLIDIVDQLKNYVNDLDPEFLPAPEDVETNCE | | |
| | | WSAFSCFQKAQLKSANTGNNERIINVSIKKLKRKPP | | |
| | | STNADRRQKHRLTCPSCDSYEKKPPKEFLERFKSLL | | |
| | | QKMIHQHLSSRTHGSEDS | | |
| IL-21_4208 | H35A, W73L, N91T, | MRSSPGNMERIVICLMVIFLGTLVHKSSSQGQDRAM | 1.796 | 746 |
| | P107L | IRMRQLIDIVDQLKNYVNDLVPEFLPAPEDVETNCE | | |
| | | LSAFSCFQKAQLKSANTGTNERIINVSIKKLKRKLP | | |
| | | STNAGRRQKHRLTCPSCDSYEKKPPKEFLERFKSLL | | |
| | | QKMIHQHLSSRTHGSEDS | | |
| IL-21_2995 | N111T, R115A | MRSSPGNMERIVICLMVIFLGTLVHKSSSQGQDRHM | 1.803 | 747 |
| | | IRMRQLIDIVDQLKNYVNDLVPEFLPAPEDVETNCE | | |
| | | WSAFSCFQKAQLKSANTGNNERIINVSIKKLKRKPP | | |
| | | STTAGRAQKHRLTCPSCDSYEKKPPKEFLERFKSLL | | |
| | | QKMIHQHLSSRTHGSEDS | | |
| IL-21_1847 | D44T, V98D, | MRSSPGNMERIVICLMVIFLGTLVHKSSSQGQDRHM | 1.808 | 748 |
| | K106R, G113L | IRMRQLITIVDQLKNYVNDLVPEFLPAPEDVETNCE | | |
| | | WSAFSCFQKAQLKSANTGNNERIINDSIKKLKRRPP | | |
| | | STNALRRQKHRLTCPSCDSYEKKPPKEFLERFKSLL | | |
| | | QKMIHQHLSSRTHGSEDS | | |
| IL-21_3079 | T89A, H118D | MRSSPGNMERIVICLMVIFLGTLVHKSSSQGQDRHM | 1.809 | 749 |
| | | IRMRQLIDIVDQLKNYVNDLVPEFLPAPEDVETNCE | | |
| | | WSAFSCFQKAQLKSANAGNNERIINVSIKKLKRKPP | | |
| | | STNAGRRQKDRLTCPSCDSYEKKPPKEFLERFKSLL | | |
| | | QKMIHQHLSSRTHGSEDS | | |
| IL-21_1034 | N51H, T69L, R114A | MRSSPGNMERIVICLMVIFLGTLVHKSSSQGQDRHM | 1.811 | 750 |
| | | IRMRQLIDIVDQLKHYVNDLVPEFLPAPEDVELNCE | | |
| | | WSAFSCFQKAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_3490 | W73Y, R94T, R114L | MRSSPGNMERIVICLMVIFLGTLVHKSSSQGQDRHM | 1.812 | 751 |
| | | IRMRQLIDIVDQLKNYVNDLVPEFLPAPEDVETNCE | | |
| | | YSAFSCFQKAQLKSANTGNNETIINVSIKKLKRKPP | | |
| | | STNAGLRQKHRLTCPSCDSYEKKPPKEFLERFKSLL | | |
| | | QKMIHQHLSSRTHGSEDS | | |
| IL-21_2713 | Q32N, R114A | MRSSPGNMERIVICLMVIFLGTLVHKSSSQGNDRHM | 1.815 | 752 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_4193 | M36V, R38H, E68L, | MRSSPGNMERIVICLMVIFLGTLVHKSSSQGQDRHV | 1.816 | 753 |
| | R114A | IHMRQLIDIVDQLKNYVNDLVPEFLPAPEDVLTNCE | | |
| | | WSAFSCFQKAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_4147 | R40E, N97D, | MRSSPGNMERIVICLMVIFLGTLVHKSSSQGQDRHM | 1.817 | 754 |
| | P107Y, N111I | IRMEQLIDIVDQLKNYVNDLVPEFLPAPEDVETNCE | | |
| | | WSAFSCFQKAQLKSANTGNNERIIDVSIKKLKRKYP | | |
| | | STIAGRRQKHRLTCPSCDSYEKKPPKEFLERFKSLL | | |
| | | QKMIHQHLSSRTHGSEDS | | |
| IL-21_3613 | P58T, P107L, R114N | MRSSPGNMERIVICLMVIFLGTLVHKSSSQGQDRHM | 1.822 | 755 |
| | | IRMRQLIDIVDQLKNYVNDLVTEFLPAPEDVETNCE | | |
| | | WSAFSCFQKAQLKSANTGNNERIINVSIKKLKRKLP | | |
| | | STNAGNRQKHRLTCPSCDSYEKKPPKEFLERFKSLL | | |
| | | QKMIHQHLSSRTHGSEDS | | |
| IL-21_2971 | Q32D, R40L | MRSSPGNMERIVICLMVIFLGTLVHKSSSQGDDRHM | 1.822 | 756 |
| | | IRMLQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4044 | W73P, K106F, | MRSSPGNMERIVICLMVIFLGTLVHKSSSQGQDRHM | 1.823 | 757 |
| | S109P, R114A | IRMRQLIDIVDQLKNYVNDLVPEFLPAPEDVETNCE | | |
| | | PSAFSCFQKAQLKSANTGNNERIINVSIKKLKRFPP | | |
| | | PTNAGARQKHRLTCPSCDSYEKKPPKEFLERFKSLL | | |
| | | QKMIHQHLSSRTHGSEDS | | |
| IL-21_2772 | E72L, R115G | MRSSPGNMERIVICLMVIFLGTLVHKSSSQGQDRHM | 1.826 | 758 |
| | | IRMRQLIDIVDQLKNYVNDLVPEFLPAPEDVETNCL | | |
| | | WSAFSCFQKAQLKSANTGNNERIINVSIKKLKRKPP | | |
| | | STNAGRGQKHRLTCPSCDSYEKKPPKEFLERFKSLL | | |
| | | QKMIHQHLSSRTHGSEDS | | |
| IL-21_0576 | Q48E, N54S | MRSSPGNMERIVICLMVIFLGTLVHKSSSQGQDRHM | 1.826 | 759 |
| | | IRMRQLIDIVDELKNYVSDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0308 | I45T, Q48E | MRSSPGNMERIVICLMVIFLGTLVHKSSSQGQDRHM | 1.826 | 760 |
| | | IRMRQLIDTVDELKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2978 | R119K, T121D | MRSSPGNMERIVICLMVIFLGTLVHKSSSQGQDRHM | 1.833 | 761 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHKLDCPSCDSYEKKPPKEFLERFKSLL | | |
| | | QKMIHQHLSSRTHGSEDS | | |
| IL-21_3512 | R40A, V57E, R115K | MRSSPGNMERIVICLMVIFLGTLVHKSSSQGQDRHM | 1.833 | 762 |
| | | IRMAQLIDIVDQLKNYVNDLEPEFLPAPEDVETNCE | | |
| | | WSAFSCFQKAQLKSANTGNNERIINVSIKKLKRKPP | | |
| | | STNAGRKQKHRLTCPSCDSYEKKPPKEFLERFKSLL | | |
| | | QKMIHQHLSSRTHGSEDS | | |
| IL-21_2770 | R114A, Q116S | MRSSPGNMERIVICLMVIFLGTLVHKSSSQGQDRHM | 1.838 | 763 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGARSKHRLTCPSCDSYEKKPPKEFLERFKSLL | | |
| | | QKMIHQHLSSRTHGSEDS | | |
| IL-21_2802 | N111Y, R115G | MRSSPGNMERIVICLMVIFLGTLVHKSSSQGQDRHM | 1.845 | 764 |
| | | IRMRQLIDIVDQLKNYVNDLVPEFLPAPEDVETNCE | | |
| | | WSAFSCFQKAQLKSANTGNNERIINVSIKKLKRKPP | | |
| | | STYAGRGQKHRLTCPSCDSYEKKPPKEFLERFKSLL | | |
| | | QKMIHQHLSSRTHGSEDS | | |
| IL-21_4103 | Q32A, H35F, V46I, | MRSSPGNMERIVICLMVIFLGTLVHKSSSQGADREM | 1.852 | 765 |
| | D47E | IRMRQLIDIIEQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2899 | R114V, L120S | MRSSPGNMERIVICLMVIFLGTLVHKSSSQGQDRHM | 1.86 | 766 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGVRQKHRSTCPSCDSYEKKPPKEFLERFKSLL | | |
| | | QKMIHQHLSSRTHGSEDS | | |
| IL-21_3508 | R40S, I43L, R115E | MRSSPGNMERIVICLMVIFLGTLVHKSSSQGQDRHM | 1.866 | 767 |
| | | IRMSQLLDIVDQLKNYVNDLVPEFLPAPEDVETNCE | | |
| | | WSAFSCFQKAQLKSANTGNNERIINVSIKKLKRKPP | | |
| | | STNAGREQKHRLTCPSCDSYEKKPPKEFLERFKSLL | | |
| | | QKMIHQHLSSRTHGSEDS | | |
| IL-21_2753 | H35S, K102L | MRSSPGNMERIVICLMVIFLGTLVHKSSSQGQDRSM | 1.867 | 768 |
| | | IRMRQLIDIVDQLKNYVNDLVPEFLPAPEDVETNCE | | |
| | | WSAFSCFQKAQLKSANTGNNERIINVSIKLLKRKPP | | |
| | | STNAGRRQKHRLTCPSCDSYEKKPPKEFLERFKSLL | | |
| | | QKMIHQHLSSRTHGSEDS | | |
| IL-21_3702 | P107L, S109I, T121P | MRSSPGNMERIVICLMVIFLGTLVHKSSSQGQDRHM | 1.869 | 769 |
| | | IRMRQLIDIVDQLKNYVNDLVPEFLPAPEDVETNCE | | |
| | | WSAFSCFQKAQLKSANTGNNERIINVSIKKLKRKLP | | |
| | | ITNAGRRQKHRLPCPSCDSYEKKPPKEFLERFKSLL | | |
| | | QKMIHQHLSSRTHGSEDS | | |
| IL-21_3665 | R38A, V57E, R114D | MRSSPGNMERIVICLMVIFLGTLVHKSSSQGQDRHM | 1.869 | 770 |
| | | IAMRQLIDIVDQLKNYVNDLEPEFLPAPEDVETNCE | | |
| | | WSAFSCFQKAQLKSANTGNNERIINVSIKKLKRKPP | | |
| | | STNAGDRQKHRLTCPSCDSYEKKPPKEFLERFKSLL | | |
| | | QKMIHQHLSSRTHGSEDS | | |
| IL-21_4304 | H35Q, M36E, V53L, | MRSSPGNMERIVICLMVIFLGTLVHKSSSQGQDRQE | 1.87 | 771 |
| | P107I | IRMRQLIDIVDQLKNYLNDLVPEFLPAPEDVETNCE | | |
| | | WSAFSCFQKAQLKSANTGNNERIINVSIKKLKRKIP | | |
| | | STNAGRRQKHRLTCPSCDSYEKKPPKEFLERFKSLL | | |
| | | QKMIHQHLSSRTHGSEDS | | |
| IL-21_3080 | S77A, R114S | MRSSPGNMERIVICLMVIFLGTLVHKSSSQGQDRHM | 1.87 | 772 |
| | | IRMRQLIDIVDQLKNYVNDLVPEFLPAPEDVETNCE | | |
| | | WSAFACFQKAQLKSANTGNNERIINVSIKKLKRKPP | | |
| | | STNAGSRQKHRLTCPSCDSYEKKPPKEFLERFKSLL | | |
| | | QKMIHQHLSSRTHGSEDS | | |
| IL-21_2816 | T69R, R94Q | MRSSPGNMERIVICLMVIFLGTLVHKSSSQGQDRHM | 1.873 | 773 |
| | | IRMRQLIDIVDQLKNYVNDLVPEFLPAPEDVERNCE | | |
| | | WSAFSCFQKAQLKSANTGNNEQIINVSIKKLKRKPP | | |
| | | STNAGRRQKHRLTCPSCDSYEKKPPKEFLERFKSLL | | |
| | | QKMIHQHLSSRTHGSEDS | | |
| IL-21_1632 | I45T, K81N, R94Q, | MRSSPGNMERIVICLMVIFLGTLVHKSSSQGQDRHM | 1.876 | 774 |
| | S109K | IRMRQLIDTVDQLKNYVNDLVPEFLPAPEDVETNCE | | |
| | | WSAFSCFQNAQLKSANTGNNEQIINVSIKKLKRKPP | | |
| | | KTNAGRRQKHRLTCPSCDSYEKKPPKEFLERFKSLL | | |
| | | QKMIHQHLSSRTHGSEDS | | |
| IL-21_3361 | R40M, V57E, | MRSSPGNMERIVICLMVIFLGTLVHKSSSQGQDRHM | 1.877 | 775 |
| | G113N | IRMMQLIDIVDQLKNYVNDLEPEFLPAPEDVETNCE | | |
| | | WSAFSCFQKAQLKSANTGNNERIINVSIKKLKRKPP | | |
| | | STNANRRQKHRLTCPSCDSYEKKPPKEFLERFKSLL | | |
| | | QKMIHQHLSSRTHGSEDS | | |
| IL-21_3036 | T89A, R94T | MRSSPGNMERIVICLMVIFLGTLVHKSSSQGQDRHM | 1.877 | 776 |
| | | IRMRQLIDIVDQLKNYVNDLVPEFLPAPEDVETNCE | | |
| | | WSAFSCFQKAQLKSANAGNNETIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2956 | R115K, T121E | MRSSPGNMERIVICLMVIFLGTLVHKSSSQGQDRHM | 1.883 | 777 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRKQKHRLECPSCDSYEKKPPKEFLERFKSLL | | |
| | | QKMIHQHLSSRTHGSEDS | | |
| IL-21_4669 | N70G, T89P, N97K, | MRSSPGNMERIVICLMVIFLGTLVHKSSSQGQDRHM | 1.885 | 778 |
| | S99L, K102A | IRMRQLIDIVDQLKNYVNDLVPEFLPAPEDVETGCE | | |
| | | WSAFSCFQKAQLKSANPGNNERIIKVLIKALKRKPP | | |
| | | STNAGRRQKHRLTCPSCDSYEKKPPKEFLERFKSLL | | |
| | | QKMIHQHLSSRTHGSEDS | | |
| IL-21_2869 | N70H, R114I | MRSSPGNMERIVICLMVIFLGTLVHKSSSQGQDRHM | 1.887 | 779 |
| | | IRMRQLIDIVDQLKNYVNDLVPEFLPAPEDVETHCE | | |
| | | WSAFSCFQKAQLKSANTGNNERIINVSIKKLKRKPP | | |
| | | STNAGIRQKHRLTCPSCDSYEKKPPKEFLERFKSLL | | |
| | | QKMIHQHLSSRTHGSEDS | | |
| IL-21_4283 | D33F, H35F, S74A, | MRSSPGNMERIVICLMVIFLGTLVHKSSSQGQFRFM | 1.89 | 780 |
| | Q83E | IRMRQLIDIVDQLKNYVNDLVPEFLPAPEDVETNCE | | |
| | | WAAFSCFQKAELKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_1359 | K50S, E59D, K106S, | MRSSPGNMERIVICLMVIFLGTLVHKSSSQGQDRHM | 1.892 | 781 |
| | T110N | IRMRQLIDIVDQLSNYVNDLVPDELPAPEDVETNCE | | |
| | | WSAFSCFQKAQLKSANTGNNERIINVSIKKLKRSPP | | |
| | | SNNAGRRQKHRLTCPSCDSYEKKPPKEFLERFKSLL | | |
| | | QKMIHQHLSSRTHGSEDS | | |
| IL-21_4189 | Q32V, F60Q, | MRSSPGNMERIVICLMVIFLGTLVHKSSSQGVDRHM | 1.902 | 782 |
| | A112E, G113N | IRMRQLIDIVDQLKNYVNDLVPEQLPAPEDVETNCE | | |
| | | WSAFSCFQKAQLKSANTGNNERIINVSIKKLKRKPP | | |
| | | STNENRRQKHRLTCPSCDSYEKKPPKEFLERFKSLL | | |
| | | QKMIHQHLSSRTHGSEDS | | |
| IL-21_3087 | R114G, Q116Y | MRSSPGNMERIVICLMVIFLGTLVHKSSSQGQDRHM | 1.909 | 783 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGGRYKHRLTCPSCDSYEKKPPKEFLERFKSLL | | |
| | | QKMIHQHLSSRTHGSEDS | | |
| IL-21_2796 | V57G, R114G | MRSSPGNMERIVICLMVIFLGTLVHKSSSQGQDRHM | 1.91 | 784 |
| | | IRMRQLIDIVDQLKNYVNDLGPEFLPAPEDVETNCE | | |
| | | WSAFSCFQKAQLKSANTGNNERIINVSIKKLKRKPP | | |
| | | STNAGGRQKHRLTCPSCDSYEKKPPKEFLERFKSLL | | |
| | | QKMIHQHLSSRTHGSEDS | | |
| IL-21_2880 | P107E, R114A | MRSSPGNMERIVICLMVIFLGTLVHKSSSQGQDRHM | 1.916 | 785 |
| | | IRMRQLIDIVDQLKNYVNDLVPEFLPAPEDVETNCE | | |
| | | WSAFSCFQKAQLKSANTGNNERIINVSIKKLKRKEP | | |
| | | STNAGARQKHRLTCPSCDSYEKKPPKEFLERFKSLL | | |
| | | QKMIHQHLSSRTHGSEDS | | |
| IL-21_1585 | Q32L, R34P, I45E, | MRSSPGNMERIVICLMVIFLGTLVHKSSSQGLDPHM | 1.917 | 786 |
| | R114Y | IRMRQLIDEVDQLKNYVNDLVPEFLPAPEDVETNCE | | |
| | | WSAFSCFQKAQLKSANTGNNERIINVSIKKLKRKPP | | |
| | | STNAGYRQKHRLTCPSCDSYEKKPPKEFLERFKSLL | | |
| | | QKMIHQHLSSRTHGSEDS | | |
| IL-21_3602 | R40T, N70H, R115F | MRSSPGNMERIVICLMVIFLGTLVHKSSSQGQDRHM | 1.917 | 787 |
| | | IRMTQLIDIVDQLKNYVNDLVPEFLPAPEDVETHCE | | |
| | | WSAFSCFQKAQLKSANTGNNERIINVSIKKLKRKPP | | |
| | | STNAGRFQKHRLTCPSCDSYEKKPPKEFLERFKSLL | | |
| | | QKMIHQHLSSRTHGSEDS | | |
| IL-21_2820 | W73L, P107L | MRSSPGNMERIVICLMVIFLGTLVHKSSSQGQDRHM | 1.918 | 788 |
| | | IRMRQLIDIVDQLKNYVNDLVPEFLPAPEDVETNCE | | |
| | | LSAFSCFQKAQLKSANTGNNERIINVSIKKLKRKLP | | |
| | | STNAGRRQKHRLTCPSCDSYEKKPPKEFLERFKSLL | | |
| | | QKMIHQHLSSRTHGSEDS | | |
| IL-21_4092 | R40L, Y52V, T69R, | MRSSPGNMERIVICLMVIFLGTLVHKSSSQGQDRHM | 1.92 | 789 |
| | R114Q | IRMLQLIDIVDQLKNVVNDLVPEFLPAPEDVERNCE | | |
| | | WSAFSCFQKAQLKSANTGNNERIINVSIKKLKRKPP | | |
| | | STNAGQRQKHRLTCPSCDSYEKKPPKEFLERFKSLL | | |
| | | QKMIHQHLSSRTHGSEDS | | |
| IL-21_3345 | N111P, G113T, | MRSSPGNMERIVICLMVIFLGTLVHKSSSQGQDRHM | 1.925 | 790 |
| | T121E | IRMRQLIDIVDQLKNYVNDLVPEFLPAPEDVETNCE | | |
| | | WSAFSCFQKAQLKSANTGNNERIINVSIKKLKRKPP | | |
| | | STPATRRQKHRLECPSCDSYEKKPPKEFLERFKSLL | | |
| | | QKMIHQHLSSRTHGSEDS | | |
| IL-21_2893 | Q32S, N111E | MRSSPGNMERIVICLMVIFLGTLVHKSSSQGSDRHM | 1.927 | 791 |
| | | IRMRQLIDIVDQLKNYVNDLVPEFLPAPEDVETNCE | | |
| | | WSAFSCFQKAQLKSANTGNNERIINVSIKKLKRKPP | | |
| | | STEAGRRQKHRLTCPSCDSYEKKPPKEFLERFKSLL | | |
| | | QKMIHQHLSSRTHGSEDS | | |
| IL-21_4771 | M36V, Y52M, S99T, | MRSSPGNMERIVICLMVIFLGTLVHKSSSQGQDRHV | 1.927 | 792 |
| | N111L, R115Q | IRMRQLIDIVDQLKNMVNDLVPEFLPAPEDVETNCE | | |
| | | WSAFSCFQKAQLKSANTGNNERIINVTIKKLKRKPP | | |
| | | STLAGRQQKHRLTCPSCDSYEKKPPKEFLERFKSLL | | |
| | | QKMIHQHLSSRTHGSEDS | | |
| IL-21_1690 | K50G, T89L, | MRSSPGNMERIVICLMVIFLGTLVHKSSSQGQDRHM | 1.93 | 793 |
| | G113M, R114Y | IRMRQLIDIVDQLGNYVNDLVPEFLPAPEDVETNCE | | |
| | | WSAFSCFQKAQLKSANLGNNERIINVSIKKLKRKPP | | |
| | | STNAMYRQKHRLTCPSCDSYEKKPPKEFLERFKSLL | | |
| | | QKMIHQHLSSRTHGSEDS | | |
| IL-21_2826 | S109T, R114A | MRSSPGNMERIVICLMVIFLGTLVHKSSSQGQDRHM | 1.933 | 794 |
| | | IRMRQLIDIVDQLKNYVNDLVPEFLPAPEDVETNCE | | |
| | | WSAFSCFQKAQLKSANTGNNERIINVSIKKLKRKPP | | |
| | | TTNAGARQKHRLTCPSCDSYEKKPPKEFLERFKSLL | | |
| | | QKMIHQHLSSRTHGSEDS | | |
| IL-21_2795 | D33E, N97D | MRSSPGNMERIVICLMVIFLGTLVHKSSSQGQERHM | 1.934 | 795 |
| | | IRMRQLIDIVDQLKNYVNDLVPEFLPAPEDVETNCE | | |
| | | WSAFSCFQKAQLKSANTGNNERIIDVSIKKLKRKPP | | |
| | | STNAGRRQKHRLTCPSCDSYEKKPPKEFLERFKSLL | | |
| | | QKMIHQHLSSRTHGSEDS | | |
| IL-21_0515 | V53S, K101Q | MRSSPGNMERIVICLMVIFLGTLVHKSSSQGQDRHM | 1.938 | 796 |
| | | IRMRQLIDIVDQLKNYSNDLVPEFLPAPEDVETNCE | | |
| | | WSAFSCFQKAQLKSANTGNNERIINVSIQKLKRKPP | | |
| | | STNAGRRQKHRLTCPSCDSYEKKPPKEFLERFKSLL | | |
| | | QKMIHQHLSSRTHGSEDS | | |
| IL-21_3362 | E72L, N111M, | MRSSPGNMERIVICLMVIFLGTLVHKSSSQGQDRHM | 1.942 | 797 |
| | R114A | IRMRQLIDIVDQLKNYVNDLVPEFLPAPEDVETNCL | | |
| | | WSAFSCFQKAQLKSANTGNNERIINVSIKKLKRKPP | | |
| | | STMAGARQKHRLTCPSCDSYEKKPPKEFLERFKSLL | | |
| | | QKMIHQHLSSRTHGSEDS | | |
| IL-21_4615 | M36P, E68A, E72L, | MRSSPGNMERIVICLMVIFLGTLVHKSSSQGQDRHP | 1.944 | 798 |
| | R114D, R115I | IRMRQLIDIVDQLKNYVNDLVPEFLPAPEDVATNCL | | |
| | | WSAFSCFQKAQLKSANTGNNERIINVSIKKLKRKPP | | |
| | | STNAGDIQKHRLTCPSCDSYEKKPPKEFLERFKSLL | | |
| | | QKMIHQHLSSRTHGSEDS | | |
| IL-21_2738 | S109A, R114A | MRSSPGNMERIVICLMVIFLGTLVHKSSSQGQDRHM | 1.946 | 799 |
| | | IRMRQLIDIVDQLKNYVNDLVPEFLPAPEDVETNCE | | |
| | | WSAFSCFQKAQLKSANTGNNERIINVSIKKLKRKPP | | |
| | | ATNAGARQKHRLTCPSCDSYEKKPPKEFLERFKSLL | | |
| | | QKMIHQHLSSRTHGSEDS | | |
| IL-21_3503 | V98T, K106P, | MRSSPGNMERIVICLMVIFLGTLVHKSSSQGQDRHM | 1.947 | 800 |
| | H118Q | IRMRQLIDIVDQLKNYVNDLVPEFLPAPEDVETNCE | | |
| | | WSAFSCFQKAQLKSANTGNNERIINTSIKKLKRPPP | | |
| | | STNAGRRQKQRLTCPSCDSYEKKPPKEFLERFKSLL | | |
| | | QKMIHQHLSSRTHGSEDS | | |
| IL-21_3474 | N97E, G113E, | MRSSPGNMERIVICLMVIFLGTLVHKSSSQGQDRHM | 1.948 | 801 |
| | Q116K | IRMRQLIDIVDQLKNYVNDLVPEFLPAPEDVETNCE | | |
| | | WSAFSCFQKAQLKSANTGNNERIIEVSIKKLKRKPP | | |
| | | STNAERRKKHRLTCPSCDSYEKKPPKEFLERFKSLL | | |
| | | QKMIHQHLSSRTHGSEDS | | |
| IL-21_0777 | K50S, L61M, A112V | MRSSPGNMERIVICLMVIFLGTLVHKSSSQGQDRHM | 1.952 | 802 |
| | | IRMRQLIDIVDQLSNYVNDLVPEEMPAPEDVETNCE | | |
| | | WSAFSCFQKAQLKSANTGNNERIINVSIKKLKRKPP | | |
| | | STNVGRRQKHRLTCPSCDSYEKKPPKEFLERFKSLL | | |
| | | QKMIHQHLSSRTHGSEDS | | |
| IL-21_3424 | P58V, N91E, P107L | MRSSPGNMERIVICLMVIFLGTLVHKSSSQGQDRHM | 1.954 | 803 |
| | | IRMRQLIDIVDQLKNYVNDLVVEFLPAPEDVETNCE | | |
| | | WSAFSCFQKAQLKSANTGENERIINVSIKKLKRKLP | | |
| | | STNAGRRQKHRLTCPSCDSYEKKPPKEFLERFKSLL | | |
| | | QKMIHQHLSSRTHGSEDS | | |
| IL-21_3001 | Q32P, H35G | MRSSPGNMERIVICLMVIFLGTLVHKSSSQGPDRGM | 1.969 | 804 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4801 | N70S, N111G, | MRSSPGNMERIVICLMVIFLGTLVHKSSSQGQDRHM | 1.969 | 805 |
| | A112G, R114G, | IRMRQLIDIVDQLKNYVNDLVPEFLPAPEDVETSCE | | |
| | K134L | WSAFSCFQKAQLKSANTGNNERIINVSIKKLKRKPP | | |
| | | STGGGGRQKHRLTCPSCDSYEKKPPLEFLERFKSLL | | |
| | | QKMIHQHLSSRTHGSEDS | | |
| IL-21_3992 | Q32F, M36I, E59D, | MRSSPGNMERIVICLMVIFLGTLVHKSSSQGEDRHI | 1.97 | 806 |
| | N111P | IRMRQLIDIVDQLKNYVNDLVPDELPAPEDVETNCE | | |
| | | WSAFSCFQKAQLKSANTGNNERIINVSIKKLKRKPP | | |
| | | STPAGRRQKHRLTCPSCDSYEKKPPKEFLERFKSLL | | |
| | | QKMIHQHLSSRTHGSEDS | | |
| IL-21_3431 | I37M, T69R, R114E | MRSSPGNMERIVICLMVIFLGTLVHKSSSQGQDRHM | 1.972 | 807 |
| | | MRMRQLIDIVDQLKNYVNDLVPEFLPAPEDVERNCE | | |
| | | WSAFSCFQKAQLKSANTGNNERIINVSIKKLKRKPP | | |
| | | STNAGERQKHRLTCPSCDSYEKKPPKEFLERFKSLL | | |
| | | QKMIHQHLSSRTHGSEDS | | |
| IL-21_3664 | A82G, S99A, T121Q | MRSSPGNMERIVICLMVIFLGTLVHKSSSQGQDRHM | 1.974 | 808 |
| | | IRMRQLIDIVDQLKNYVNDLVPEFLPAPEDVETNCE | | |
| | | WSAFSCFQKGQLKSANTGNNERIINVAIKKLKRKPP | | |
| | | STNAGRRQKHRLQCPSCDSYEKKPPKEFLERFKSLL | | |
| | | QKMIHQHLSSRTHGSEDS | | |
| IL-21_1415 | R40K, I45S, K81G, | MRSSPGNMERIVICLMVIFLGTLVHKSSSQGQDRHM | 1.975 | 809 |
| | R115T | IRMKQLIDSVDQLKNYVNDLVPEFLPAPEDVETNCE | | |
| | | WSAFSCFQGAQLKSANTGNNERIINVSIKKLKRKPP | | |
| | | STNAGRTQKHRLTCPSCDSYEKKPPKEFLERFKSLL | | |
| | | QKMIHQHLSSRTHGSEDS | | |
| IL-21_3436 | T69K, S99L, N111M | MRSSPGNMERIVICLMVIFLGTLVHKSSSQGQDRHM | 1.982 | 810 |
| | | IRMRQLIDIVDQLKNYVNDLVPEFLPAPEDVEKNCE | | |
| | | WSAFSCFQKAQLKSANTGNNERIINVLIKKLKRKPP | | |
| | | STMAGRRQKHRLTCPSCDSYEKKPPKEFLERFKSLL | | |
| | | QKMIHQHLSSRTHGSEDS | | |
| IL-21_3651 | S77L, R114A, | MRSSPGNMERIVICLMVIFLGTLVHKSSSQGQDRHM | 1.987 | 811 |
| | L120Y | IRMRQLIDIVDQLKNYVNDLVPEFLPAPEDVETNCE | | |
| | | WSAFLCFQKAQLKSANTGNNERIINVSIKKLKRKPP | | |
| | | STNAGARQKHRYTCPSCDSYEKKPPKEFLERFKSLL | | |
| | | QKMIHQHLSSRTHGSEDS | | |
| IL-21_2884 | V57D, T89G | MRSSPGNMERIVICLMVIFLGTLVHKSSSQGQDRHM | 1.987 | 812 |
| | | IRMRQLIDIVDQLKNYVNDLDPEFLPAPEDVETNCE | | |
| | | WSAFSCFQKAQLKSANGGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0107 | K50S, E59D | MRSSPGNMERIVICLMVIFLGTLVHKSSSQGQDRHM | 1.99 | 813 |
| | | IRMRQLIDIVDQLSNYVNDLVPDELPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_1384 | H35L, I43S, P108I, | MRSSPGNMERIVICLMVIFLGTLVHKSSSQGQDRLM | 1.995 | 814 |
| | R114E | IRMRQLSDIVDQLKNYVNDLVPEFLPAPEDVETNCE | | |
| | | WSAFSCFQKAQLKSANTGNNERIINVSIKKLKRKPI | | |
| | | STNAGERQKHRLTCPSCDSYEKKPPKEFLERFKSLL | | |
| | | QKMIHQHLSSRTHGSEDS | | |
| IL-21_0377 | N51H, S74E | MRSSPGNMERIVICLMVIFLGTLVHKSSSQGQDRHM | 1.998 | 815 |
| | | IRMRQLIDIVDQLKHYVNDLVPEFLPAPEDVETNCE | | |
| | | WEAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4192 | H35Q, Y52M, N70E, | MRSSPGNMERIVICLMVIFLGTLVHKSSSQGQDRQM | 1.999 | 816 |
| | N91G | IRMRQLIDIVDQLKNMVNDLVPEFLPAPEDVETECE | | |
| | | WSAFSCFQKAQLKSANTGGNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3711 | Q32V, D33V, K102E | MRSSPGNMERIVICLMVIFLGTLVHKSSSQGVVRHM | 2 | 817 |
| | | IRMRQLIDIVDQLKNYVNDLVPEFLPAPEDVETNCE | | |
| | | WSAFSCFQKAQLKSANTGNNERIINVSIKELKRKPP | | |
| | | STNAGRRQKHRLTCPSCDSYEKKPPKEFLERFKSLL | | |
| | | QKMIHQHLSSRTHGSEDS | | |
| IL-21_0050 | I45T, F60Q | MRSSPGNMERIVICLMVIFLGTLVHKSSSQGQDRHM | 2.001 | 818 |
| | | IRMRQLIDTVDQLKNYVNDLVPEQLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4871 | R40K, V57M, K81R, | MRSSPGNMERIVICLMVIFLGTLVHKSSSQGQDRHM | 2.01 | 819 |
| | V98I, P107I | IRMKQLIDIVDQLKNYVNDLMPEFLPAPEDVETNCE | | |
| | | WSAFSCFQRAQLKSANTGNNERIINISIKKLKRKIP | | |
| | | STNAGRRQKHRLTCPSCDSYEKKPPKEFLERFKSLL | | |
| | | QKMIHQHLSSRTHGSEDS | | |
| IL-21_2875 | H35S, V98G | MRSSPGNMERIVICLMVIFLGTLVHKSSSQGQDRSM | 2.016 | 820 |
| | | IRMRQLIDIVDQLKNYVNDLVPEFLPAPEDVETNCE | | |
| | | WSAFSCFQKAQLKSANTGNNERIINGSIKKLKRKPP | | |
| | | STNAGRRQKHRLTCPSCDSYEKKPPKEFLERFKSLL | | |
| | | QKMIHQHLSSRTHGSEDS | | |
| IL-21_2664 | S99T, H118Q | MRSSPGNMERIVICLMVIFLGTLVHKSSSQGQDRHM | 2.017 | 821 |
| | | IRMRQLIDIVDQLKNYVNDLVPEFLPAPEDVETNCE | | |
| | | WSAFSCFQKAQLKSANTGNNERIINVTIKKLKRKPP | | |
| | | STNAGRRQKQRLTCPSCDSYEKKPPKEFLERFKSLL | | |
| | | QKMIHQHLSSRTHGSEDS | | |
| IL-21_0504 | V53S, S99A | MRSSPGNMERIVICLMVIFLGTLVHKSSSQGQDRHM | 2.022 | 822 |
| | | IRMRQLIDIVDQLKNYSNDLVPEFLPAPEDVETNCE | | |
| | | WSAFSCFQKAQLKSANTGNNERIINVAIKKLKRKPP | | |
| | | STNAGRRQKHRLTCPSCDSYEKKPPKEFLERFKSLL | | |
| | | QKMIHQHLSSRTHGSEDS | | |
| IL-21_2695 | R114A, L120K | MRSSPGNMERIVICLMVIFLGTLVHKSSSQGQDRHM | 2.025 | 823 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGARQKHRKTCPSCDSYEKKPPKEFLERFKSLL | | |
| | | QKMIHQHLSSRTHGSEDS | | |
| IL-21_4308 | D33A, D55E, | MRSSPGNMERIVICLMVIFLGTLVHKSSSQGQARHM | 2.025 | 824 |
| | K102Q, A112V | IRMRQLIDIVDQLKNYVNELVPEFLPAPEDVETNCE | | |
| | | WSAFSCFQKAQLKSANTGNNERIINVSIKQLKRKPP | | |
| | | STNVGRRQKHRLTCPSCDSYEKKPPKEFLERFKSLL | | |
| | | QKMIHQHLSSRTHGSEDS | | |
| IL-21_2767 | N111S, R114Y | MRSSPGNMERIVICLMVIFLGTLVHKSSSQGQDRHM | 2.039 | 825 |
| | | IRMRQLIDIVDQLKNYVNDLVPEFLPAPEDVETNCE | | |
| | | WSAFSCFQKAQLKSANTGNNERIINVSIKKLKRKPP | | |
| | | STSAGYRQKHRLTCPSCDSYEKKPPKEFLERFKSLL | | |
| | | QKMIHQHLSSRTHGSEDS | | |
| IL-21_0548 | H35S, F60A | MRSSPGNMERIVICLMVIFLGTLVHKSSSQGQDRSM | 2.04 | 826 |
| | | IRMRQLIDIVDQLKNYVNDLVPEALPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2955 | Q116E, L120Q | MRSSPGNMERIVICLMVIFLGTLVHKSSSQGQDRHM | 2.041 | 827 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRREKHRQTCPSCDSYEKKPPKEFLERFKSLL | | |
| | | QKMIHQHLSSRTHGSEDS | | |
| IL-21_3687 | T89N, P107L, | MRSSPGNMERIVICLMVIFLGTLVHKSSSQGQDRHM | 2.042 | 828 |
| | G113V | IRMRQLIDIVDQLKNYVNDLVPEFLPAPEDVETNCE | | |
| | | WSAFSCFQKAQLKSANNGNNERIINVSIKKLKRKLP | | |
| | | STNAVRRQKHRLTCPSCDSYEKKPPKEFLERFKSLL | | |
| | | QKMIHQHLSSRTHGSEDS | | |
| IL-21_2829 | D33E, Q48E | MRSSPGNMERIVICLMVIFLGTLVHKSSSQGQERHM | 2.043 | 829 |
| | | IRMRQLIDIVDELKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_1898 | I43Q, K50S, N54E, | MRSSPGNMERIVICLMVIFLGTLVHKSSSQGQDRHM | 2.048 | 830 |
| | F60N, K106I | IRMRQLQDIVDQLSNYVEDLVPENLPAPEDVETNCE | | |
| | | WSAFSCFQKAQLKSANTGNNERIINVSIKKLKRIPP | | |
| | | STNAGRRQKHRLTCPSCDSYEKKPPKEFLERFKSLL | | |
| | | QKMIHQHLSSRTHGSEDS | | |
| IL-21_2584 | I37E, R114E | MRSSPGNMERIVICLMVIFLGTLVHKSSSQGQDRHM | 2.049 | 831 |
| | | ERMRQLIDIVDQLKNYVNDLVPEFLPAPEDVETNCE | | |
| | | WSAFSCFQKAQLKSANTGNNERIINVSIKKLKRKPP | | |
| | | STNAGERQKHRLTCPSCDSYEKKPPKEFLERFKSLL | | |
| | | QKMIHQHLSSRTHGSEDS | | |
| IL-21_2951 | R34T, N111D | MRSSPGNMERIVICLMVIFLGTLVHKSSSQGQDTHM | 2.053 | 832 |
| | | IRMRQLIDIVDQLKNYVNDLVPEFLPAPEDVETNCE | | |
| | | WSAFSCFQKAQLKSANTGNNERIINVSIKKLKRKPP | | |
| | | STDAGRRQKHRLTCPSCDSYEKKPPKEFLERFKSLL | | |
| | | QKMIHQHLSSRTHGSEDS | | |
| IL-21_4063 | N91G, R114A, | MRSSPGNMERIVICLMVIFLGTLVHKSSSQGQDRHM | 2.054 | 833 |
| | Q116T, T121P | IRMRQLIDIVDQLKNYVNDLVPEFLPAPEDVETNCE | | |
| | | WSAFSCFQKAQLKSANTGGNERIINVSIKKLKRKPP | | |
| | | STNAGARTKHRLPCPSCDSYEKKPPKEFLERFKSLL | | |
| | | QKMIHQHLSSRTHGSEDS | | |
| IL-21_4728 | Q32S, W73Q, | MRSSPGNMERIVICLMVIFLGTLVHKSSSQGSDRHM | 2.057 | 834 |
| | K104G, P107L, | IRMRQLIDIVDQLKNYVNDLVPEFLPAPEDVETNCE | | |
| | N111R | QSAFSCFQKAQLKSANTGNNERIINVSIKKLGRKLP | | |
| | | STRAGRRQKHRLTCPSCDSYEKKPPKEFLERFKSLL | | |
| | | QKMIHQHLSSRTHGSEDS | | |
| IL-21_3655 | Q32I, M36N, R94N | MRSSPGNMERIVICLMVIFLGTLVHKSSSQGIDRHN | 2.06 | 835 |
| | | IRMRQLIDIVDQLKNYVNDLVPEFLPAPEDVETNCE | | |
| | | WSAFSCFQKAQLKSANTGNNENIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2708 | H35F, M36P | MRSSPGNMERIVICLMVIFLGTLVHKSSSQGQDRFP | 2.062 | 836 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0344 | N51H, R114S | MRSSPGNMERIVICLMVIFLGTLVHKSSSQGQDRHM | 2.066 | 837 |
| | | IRMRQLIDIVDQLKHYVNDLVPEFLPAPEDVETNCE | | |
| | | WSAFSCFQKAQLKSANTGNNERIINVSIKKLKRKPP | | |
| | | STNAGSRQKHRLTCPSCDSYEKKPPKEFLERFKSLL | | |
| | | QKMIHQHLSSRTHGSEDS | | |
| IL-21_3522 | Q32A, N111D, | MRSSPGNMERIVICLMVIFLGTLVHKSSSQGADRHM | 2.07 | 838 |
| | G113Q | IRMRQLIDIVDQLKNYVNDLVPEFLPAPEDVETNCE | | |
| | | WSAFSCFQKAQLKSANTGNNERIINVSIKKLKRKPP | | |
| | | STDAQRRQKHRLTCPSCDSYEKKPPKEFLERFKSLL | | |
| | | QKMIHQHLSSRTHGSEDS | | |
| IL-21_3095 | Q32A, R40H | MRSSPGNMERIVICLMVIFLGTLVHKSSSQGADRHM | 2.075 | 839 |
| | | IRMHQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_1229 | Q48L, N51T, V98N | MRSSPGNMERIVICLMVIFLGTLVHKSSSQGQDRHM | 2.078 | 840 |
| | | IRMRQLIDIVDLLKTYVNDLVPEFLPAPEDVETNCE | | |
| | | WSAFSCFQKAQLKSANTGNNERIINNSIKKLKRKPP | | |
| | | STNAGRRQKHRLTCPSCDSYEKKPPKEFLERFKSLL | | |
| | | QKMIHQHLSSRTHGSEDS | | |
| IL-21_0144 | K50S, V98I | MRSSPGNMERIVICLMVIFLGTLVHKSSSQGQDRHM | 2.079 | 841 |
| | | IRMRQLIDIVDQLSNYVNDLVPEFLPAPEDVETNCE | | |
| | | WSAFSCFQKAQLKSANTGNNERIINISIKKLKRKPP | | |
| | | STNAGRRQKHRLTCPSCDSYEKKPPKEFLERFKSLL | | |
| | | QKMIHQHLSSRTHGSEDS | | |
| IL-21_0539 | N51T, N111S | MRSSPGNMERIVICLMVIFLGTLVHKSSSQGQDRHM | 2.08 | 842 |
| | | IRMRQLIDIVDQLKTYVNDLVPEFLPAPEDVETNCE | | |
| | | WSAFSCFQKAQLKSANTGNNERIINVSIKKLKRKPP | | |
| | | STSAGRRQKHRLTCPSCDSYEKKPPKEFLERFKSLL | | |
| | | QKMIHQHLSSRTHGSEDS | | |
| IL-21_2928 | D33E, K104S | MRSSPGNMERIVICLMVIFLGTLVHKSSSQGQERHM | 2.082 | 843 |
| | | IRMRQLIDIVDQLKNYVNDLVPEFLPAPEDVETNCE | | |
| | | WSAFSCFQKAQLKSANTGNNERIINVSIKKLSRKPP | | |
| | | STNAGRRQKHRLTCPSCDSYEKKPPKEFLERFKSLL | | |
| | | QKMIHQHLSSRTHGSEDS | | |
| IL-21_2138 | R38H, I45T, P62A, | MRSSPGNMERIVICLMVIFLGTLVHKSSSQGQDRHM | 2.082 | 844 |
| | N111Y, R114Y | IHMRQLIDTVDQLKNYVNDLVPEFLAAPEDVETNCE | | |
| | | WSAFSCFQKAQLKSANTGNNERIINVSIKKLKRKPP | | |
| | | STYAGYRQKHRLTCPSCDSYEKKPPKEFLERFKSLL | | |
| | | QKMIHQHLSSRTHGSEDS | | |
| IL-21_4261 | Q32E, H35T, I37M, | MRSSPGNMERIVICLMVIFLGTLVHKSSSQGEDRTM | 2.087 | 845 |
| | N70T | MRMRQLIDIVDQLKNYVNDLVPEFLPAPEDVETTCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2371 | H35E, N51T, E59L, | MRSSPGNMERIVICLMVIFLGTLVHKSSSQGQDREM | 2.089 | 846 |
| | K106Q, G113A | IRMRQLIDIVDQLKTYVNDLVPLFLPAPEDVETNCE | | |
| | | WSAFSCFQKAQLKSANTGNNERIINVSIKKLKRQPP | | |
| | | STNAARRQKHRLTCPSCDSYEKKPPKEFLERFKSLL | | |
| | | QKMIHQHLSSRTHGSEDS | | |
| IL-21_2840 | P62H, S99L | MRSSPGNMERIVICLMVIFLGTLVHKSSSQGQDRHM | 2.09 | 847 |
| | | IRMRQLIDIVDQLKNYVNDLVPEFLHAPEDVETNCE | | |
| | | WSAFSCFQKAQLKSANTGNNERIINVLIKKLKRKPP | | |
| | | STNAGRRQKHRLTCPSCDSYEKKPPKEFLERFKSLL | | |
| | | QKMIHQHLSSRTHGSEDS | | |
| IL-21_2714 | F60L, N111S | MRSSPGNMERIVICLMVIFLGTLVHKSSSQGQDRHM | 2.091 | 848 |
| | | IRMRQLIDIVDQLKNYVNDLVPELLPAPEDVETNCE | | |
| | | WSAFSCFQKAQLKSANTGNNERIINVSIKKLKRKPP | | |
| | | STSAGRRQKHRLTCPSCDSYEKKPPKEFLERFKSLL | | |
| | | QKMIHQHLSSRTHGSEDS | | |
| IL-21_0188 | H35Y, I43S | MRSSPGNMERIVICLMVIFLGTLVHKSSSQGQDRYM | 2.093 | 849 |
| | | IRMRQLSDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3695 | N70H, R115Q, | MRSSPGNMERIVICLMVIFLGTLVHKSSSQGQDRHM | 2.094 | 850 |
| | L120A | IRMRQLIDIVDQLKNYVNDLVPEFLPAPEDVETHCE | | |
| | | WSAFSCFQKAQLKSANTGNNERIINVSIKKLKRKPP | | |
| | | STNAGRQQKHRATCPSCDSYEKKPPKEFLERFKSLL | | |
| | | QKMIHQHLSSRTHGSEDS | | |
| IL-21_4713 | N92S, K106L, | MRSSPGNMERIVICLMVIFLGTLVHKSSSQGQDRHM | 2.094 | 851 |
| | N111T, H118Q, | IRMRQLIDIVDQLKNYVNDLVPEFLPAPEDVETNCE | | |
| | I148L | WSAFSCFQKAQLKSANTGNSERIINVSIKKLKRLPP | | |
| | | STTAGRRQKQRLTCPSCDSYEKKPPKEFLERFKSLL | | |
| | | QKMLHQHLSSRTHGSEDS | | |
| IL-21_2858 | T69D, R115K | MRSSPGNMERIVICLMVIFLGTLVHKSSSQGQDRHM | 2.098 | 852 |
| | | IRMRQLIDIVDQLKNYVNDLVPEFLPAPEDVEDNCE | | |
| | | WSAFSCFQKAQLKSANTGNNERIINVSIKKLKRKPP | | |
| | | STNAGRKQKHRLTCPSCDSYEKKPPKEFLERFKSLL | | |
| | | QKMIHQHLSSRTHGSEDS | | |
| IL-21_4227 | H35S, V57K, E65N, | MRSSPGNMERIVICLMVIFLGTLVHKSSSQGQDRSM | 2.099 | 853 |
| | N91E | IRMRQLIDIVDQLKNYVNDLKPEFLPAPNDVETNCE | | |
| | | WSAFSCFQKAQLKSANTGENERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_1061 | N51H, E65Q, P107M | MRSSPGNMERIVICLMVIFLGTLVHKSSSQGQDRHM | 2.099 | 854 |
| | | IRMRQLIDIVDQLKHYVNDLVPEFLPAPQDVETNCE | | |
| | | WSAFSCFQKAQLKSANTGNNERIINVSIKKLKRKMP | | |
| | | STNAGRRQKHRLTCPSCDSYEKKPPKEFLERFKSLL | | |
| | | QKMIHQHLSSRTHGSEDS | | |
| IL-21_4057 | N91K, H118E, | MRSSPGNMERIVICLMVIFLGTLVHKSSSQGQDRHM | 2.102 | 855 |
| | L120D, E138K | IRMRQLIDIVDQLKNYVNDLVPEFLPAPEDVETNCE | | |
| | | WSAFSCFQKAQLKSANTGKNERIINVSIKKLKRKPP | | |
| | | STNAGRRQKERDTCPSCDSYEKKPPKEFLKRFKSLL | | |
| | | QKMIHQHLSSRTHGSEDS | | |
| IL-21_3460 | E72L, T89S, P107I | MRSSPGNMERIVICLMVIFLGTLVHKSSSQGQDRHM | 2.106 | 856 |
| | | IRMRQLIDIVDQLKNYVNDLVPEFLPAPEDVETNCL | | |
| | | WSAFSCFQKAQLKSANSGNNERIINVSIKKLKRKIP | | |
| | | STNAGRRQKHRLTCPSCDSYEKKPPKEFLERFKSLL | | |
| | | QKMIHQHLSSRTHGSEDS | | |
| IL-21_4064 | D33P, M36I, S86A, | MRSSPGNMERIVICLMVIFLGTLVHKSSSQGQPRHI | 2.107 | 857 |
| | R114V | IRMRQLIDIVDQLKNYVNDLVPEFLPAPEDVETNCE | | |
| | | WSAFSCFQKAQLKAANTGNNERIINVSIKKLKRKPP | | |
| | | STNAGVRQKHRLTCPSCDSYEKKPPKEFLERFKSLL | | |
| | | QKMIHQHLSSRTHGSEDS | | |
| IL-21_1338 | K50G, V57S, F60D, | MRSSPGNMERIVICLMVIFLGTLVHKSSSQGQDRHM | 2.107 | 858 |
| | A112T | IRMRQLIDIVDQLGNYVNDLSPEDLPAPEDVETNCE | | |
| | | WSAFSCFQKAQLKSANTGNNERIINVSIKKLKRKPP | | |
| | | STNTGRRQKHRLTCPSCDSYEKKPPKEFLERFKSLL | | |
| | | QKMIHQHLSSRTHGSEDS | | |
| IL-21_3037 | H118E, T121P | MRSSPGNMERIVICLMVIFLGTLVHKSSSQGQDRHM | 2.111 | 859 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKERLPCPSCDSYEKKPPKEFLERFKSLL | | |
| | | QKMIHQHLSSRTHGSEDS | | |
| IL-21_2907 | Q83E, T121K | MRSSPGNMERIVICLMVIFLGTLVHKSSSQGQDRHM | 2.118 | 860 |
| | | IRMRQLIDIVDQLKNYVNDLVPEFLPAPEDVETNCE | | |
| | | WSAFSCFQKAELKSANTGNNERIINVSIKKLKRKPP | | |
| | | STNAGRRQKHRLKCPSCDSYEKKPPKEFLERFKSLL | | |
| | | QKMIHQHLSSRTHGSEDS | | |
| IL-21_4796 | D33W, I43V, F76L, | MRSSPGNMERIVICLMVIFLGTLVHKSSSQGQWRHM | 2.119 | 861 |
| | V98S, R114T | IRMRQLVDIVDQLKNYVNDLVPEFLPAPEDVETNCE | | |
| | | WSALSCFQKAQLKSANTGNNERIINSSIKKLKRKPP | | |
| | | STNAGTRQKHRLTCPSCDSYEKKPPKEFLERFKSLL | | |
| | | QKMIHQHLSSRTHGSEDS | | |
| IL-21_0775 | D44S, P107I, R115T | MRSSPGNMERIVICLMVIFLGTLVHKSSSQGQDRHM | 2.119 | 862 |
| | | IRMRQLISIVDQLKNYVNDLVPEFLPAPEDVETNCE | | |
| | | WSAFSCFQKAQLKSANTGNNERIINVSIKKLKRKIP | | |
| | | STNAGRTQKHRLTCPSCDSYEKKPPKEFLERFKSLL | | |
| | | QKMIHQHLSSRTHGSEDS | | |
| IL-21_3699 | H35S, P58V, K106V | MRSSPGNMERIVICLMVIFLGTLVHKSSSQGQDRSM | 2.125 | 863 |
| | | IRMRQLIDIVDQLKNYVNDLVVEFLPAPEDVETNCE | | |
| | | WSAFSCFQKAQLKSANTGNNERIINVSIKKLKRVPP | | |
| | | STNAGRRQKHRLTCPSCDSYEKKPPKEFLERFKSLL | | |
| | | QKMIHQHLSSRTHGSEDS | | |
| IL-21_2690 | H35M, R114A | MRSSPGNMERIVICLMVIFLGTLVHKSSSQGQDRMM | 2.132 | 864 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGARQKHRLTCPSCDSYEKKPPKEFLERFKSLL | | |
| | | QKMIHQHLSSRTHGSEDS | | |
| IL-21_0570 | K50S, S77Y | MRSSPGNMERIVICLMVIFLGTLVHKSSSQGQDRHM | 2.14 | 865 |
| | | IRMRQLIDIVDQLSNYVNDLVPEFLPAPEDVETNCE | | |
| | | WSAFYCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_2854 | V98I, R119K | MRSSPGNMERIVICLMVIFLGTLVHKSSSQGQDRHM | 2.143 | 866 |
| | | IRMRQLIDIVDQLKNYVNDLVPEFLPAPEDVETNCE | | |
| | | WSAFSCFQKAQLKSANTGNNERIINISIKKLKRKPP | | |
| | | STNAGRRQKHKLTCPSCDSYEKKPPKEFLERFKSLL | | |
| | | QKMIHQHLSSRTHGSEDS | | |
| IL-21_2733 | T69R, R94T | MRSSPGNMERIVICLMVIFLGTLVHKSSSQGQDRHM | 2.144 | 867 |
| | | IRMRQLIDIVDQLKNYVNDLVPEFLPAPEDVERNCE | | |
| | | WSAFSCFQKAQLKSANTGNNETIINVSIKKLKRKPP | | |
| | | STNAGRRQKHRLTCPSCDSYEKKPPKEFLERFKSLL | | |
| | | QKMIHQHLSSRTHGSEDS | | |
| IL-21_3393 | D33E, G113M, | MRSSPGNMERIVICLMVIFLGTLVHKSSSQGQERHM | 2.146 | 868 |
| | R115A | IRMRQLIDIVDQLKNYVNDLVPEFLPAPEDVETNCE | | |
| | | WSAFSCFQKAQLKSANTGNNERIINVSIKKLKRKPP | | |
| | | STNAMRAQKHRLTCPSCDSYEKKPPKEFLERFKSLL | | |
| | | QKMIHQHLSSRTHGSEDS | | |
| IL-21_3334 | M36T, P107I, | MRSSPGNMERIVICLMVIFLGTLVHKSSSQGQDRHT | 2.15 | 869 |
| | G113N | IRMRQLIDIVDQLKNYVNDLVPEFLPAPEDVETNCE | | |
| | | WSAFSCFQKAQLKSANTGNNERIINVSIKKLKRKIP | | |
| | | STNANRRQKHRLTCPSCDSYEKKPPKEFLERFKSLL | | |
| | | QKMIHQHLSSRTHGSEDS | | |
| IL-21_0467 | K50G, V98I | MRSSPGNMERIVICLMVIFLGTLVHKSSSQGQDRHM | 2.153 | 870 |
| | | IRMRQLIDIVDQLGNYVNDLVPEFLPAPEDVETNCE | | |
| | | WSAFSCFQKAQLKSANTGNNERIINISIKKLKRKPP | | |
| | | STNAGRRQKHRLTCPSCDSYEKKPPKEFLERFKSLL | | |
| | | QKMIHQHLSSRTHGSEDS | | |
| IL-21_2920 | N70G, W73L | MRSSPGNMERIVICLMVIFLGTLVHKSSSQGQDRHM | 2.157 | 871 |
| | | IRMRQLIDIVDQLKNYVNDLVPEFLPAPEDVETGCE | | |
| | | LSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4139 | N54K, S77V, V98Q, | MRSSPGNMERIVICLMVIFLGTLVHKSSSQGQDRHM | 2.157 | 872 |
| | R114A | IRMRQLIDIVDQLKNYVKDLVPEFLPAPEDVETNCE | | |
| | | WSAFVCFQKAQLKSANTGNNERIINQSIKKLKRKPP | | |
| | | STNAGARQKHRLTCPSCDSYEKKPPKEFLERFKSLL | | |
| | | QKMIHQHLSSRTHGSEDS | | |
| IL-21_0405 | I45T, K81S | MRSSPGNMERIVICLMVIFLGTLVHKSSSQGQDRHM | 2.159 | 873 |
| | | IRMRQLIDTVDQLKNYVNDLVPEFLPAPEDVETNCE | | |
| | | WSAFSCFQSAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0345 | N51H, L61Y | MRSSPGNMERIVICLMVIFLGTLVHKSSSQGQDRHM | 2.162 | 874 |
| | | IRMRQLIDIVDQLKHYVNDLVPEFYPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_0772 | R40L, D44S, K102E | MRSSPGNMERIVICLMVIFLGTLVHKSSSQGQDRHM | 2.163 | 875 |
| | | IRMLQLISIVDQLKNYVNDLVPEFLPAPEDVETNCE | | |
| | | WSAFSCFQKAQLKSANTGNNERIINVSIKELKRKPP | | |
| | | STNAGRRQKHRLTCPSCDSYEKKPPKEFLERFKSLL | | |
| | | QKMIHQHLSSRTHGSEDS | | |
| IL-21_0604 | Y52H, K104E | MRSSPGNMERIVICLMVIFLGTLVHKSSSQGQDRHM | 2.165 | 876 |
| | | IRMRQLIDIVDQLKNHVNDLVPEFLPAPEDVETNCE | | |
| | | WSAFSCFQKAQLKSANTGNNERIINVSIKKLERKPP | | |
| | | STNAGRRQKHRLTCPSCDSYEKKPPKEFLERFKSLL | | |
| | | QKMIHQHLSSRTHGSEDS | | |
| IL-21_2781 | H35T, T69G | MRSSPGNMERIVICLMVIFLGTLVHKSSSQGQDRTM | 2.173 | 877 |
| | | IRMRQLIDIVDQLKNYVNDLVPEFLPAPEDVEGNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4134 | H35F, M36T, V98Q, | MRSSPGNMERIVICLMVIFLGTLVHKSSSQGQDRFT | 2.178 | 878 |
| | A112R | IRMRQLIDIVDQLKNYVNDLVPEFLPAPEDVETNCE | | |
| | | WSAFSCFQKAQLKSANTGNNERIINQSIKKLKRKPP | | |
| | | STNRGRRQKHRLTCPSCDSYEKKPPKEFLERFKSLL | | |
| | | QKMIHQHLSSRTHGSEDS | | |
| IL-21_3621 | R34G, H35T, R115H | MRSSPGNMERIVICLMVIFLGTLVHKSSSQGQDGTM | 2.179 | 879 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRHQKHRLTCPSCDSYEKKPPKEFLERFKSLL | | |
| | | QKMIHQHLSSRTHGSEDS | | |
| IL-21_3033 | N70G, S77A | MRSSPGNMERIVICLMVIFLGTLVHKSSSQGQDRHM | 2.18 | 880 |
| | | IRMRQLIDIVDQLKNYVNDLVPEFLPAPEDVETGCE | | |
| | | WSAFACFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |
| IL-21_4747 | M36A, V57A, E59T, | MRSSPGNMERIVICLMVIFLGTLVHKSSSQGQDRHA | 2.182 | 881 |
| | F60L, V98L | IRMRQLIDIVDQLKNYVNDLAPTLLPAPEDVETNCE | | |
| | | WSAFSCFQKAQLKSANTGNNERIINLSIKKLKRKPP | | |
| | | STNAGRRQKHRLTCPSCDSYEKKPPKEFLERFKSLL | | |
| | | QKMIHQHLSSRTHGSEDS | | |
| IL-21_2941 | N97T, R114V | MRSSPGNMERIVICLMVIFLGTLVHKSSSQGQDRHM | 2.185 | 882 |
| | | IRMRQLIDIVDQLKNYVNDLVPEFLPAPEDVETNCE | | |
| | | WSAFSCFQKAQLKSANTGNNERIITVSIKKLKRKPP | | |
| | | STNAGVRQKHRLTCPSCDSYEKKPPKEFLERFKSLL | | |
| | | QKMIHQHLSSRTHGSEDS | | |
| IL-21_2693 | H35S, M36T | MRSSPGNMERIVICLMVIFLGTLVHKSSSQGQDRST | 2.188 | 883 |
| | | IRMRQLIDIVDQLKNYVNDLVPEFLPAPEDVETNCE | | |
| | | WSAFSCFQKAQLKSANTGNNERIINVSIKKLKRKPP | | |
| | | STNAGRRQKHRLTCPSCDSYEKKPPKEFLERFKSLL | | |
| | | QKMIHQHLSSRTHGSEDS | | |

TABLE 10
IL-21 Combination Mutant Stabilized Variants

| | | | | SEQ |
|---|---|---|---|---|
| Variant | | | Predicted | ID |
| Name | Mutations | Amino Acid Sequence | Affinity | NO |
| IL-21_5995 | H35F, K81D, | MRSSPGNMERIVICLMVIFLGTLVHKSSSQGQDRFMIRMRQ | 0.421 | 884 |
| | R114G, R115D | LIDIVDQLKNYVNDLVPEFLPAPEDVETNCEWSAFSCFQDA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_6003 | H35F, K81D, | MRSSPGNMERIVICLMVIFLGTLVHKSSSQGQDREMIRMRQ | 0.445 | 885 |
| | T110D, R115D | LIDIVDQLKNYVNDLVPEFLPAPEDVETNCEWSAFSCFQDA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_5503 | N70G, K81D, | MRSSPGNMERIVICLMVIFLGTLVHKSSSQGQDRHMIRMRQ | 0.464 | 886 |
| | V98E, R115D | LIDIVDQLKNYVNDLVPEFLPAPEDVETGCEWSAFSCFQDA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5303 | K81D, V98E, | MRSSPGNMERIVICLMVIFLGTLVHKSSSQGQDRHMIRMRQ | 0.493 | 887 |
| | P107V, R115G | LIDIVDQLKNYVNDLVPEFLPAPEDVETNCEWSAFSCFQDA | | |
| | | QLKSANTGNNERIINESIKKLKRKVPSTNAGRGQKHRLTCP | | |
| | | SCDSYEKKPPKEFLERFKSLLQKMIHQHLSSRTHGSEDS | | |
| IL-21_5502 | N70G, K81D, | MRSSPGNMERIVICLMVIFLGTLVHKSSSQGQDRHMIRMRQ | 0.502 | 888 |
| | V98E, R115G | LIDIVDQLKNYVNDLVPEFLPAPEDVETGCEWSAFSCFQDA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_5263 | K81D, P107V, | MRSSPGNMERIVICLMVIFLGTLVHKSSSQGQDRHMIRMRQ | 0.513 | 889 |
| | T110D, R114G | LIDIVDQLKNYVNDLVPEFLPAPEDVETNCEWSAFSCFQDA | | |
| | | QLKSANTGNNERIINVSIKKLKRKVPSDNAGGRQKHRLTCP | | |
| | | SCDSYEKKPPKEFLERFKSLLQKMIHQHLSSRTHGSEDS | | |
| IL-21_6013 | H35F, K81D, | MRSSPGNMERIVICLMVIFLGTLVHKSSSQGQDRFMIRMRQ | 0.516 | 890 |
| | V98E, R115G | LIDIVDQLKNYVNDLVPEFLPAPEDVETNCEWSAFSCFQDA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_5271 | K81D, V98E, | MRSSPGNMERIVICLMVIFLGTLVHKSSSQGQDRHMIRMRQ | 0.518 | 891 |
| | R115G, H118D | LIDIVDQLKNYVNDLVPEFLPAPEDVETNCEWSAFSCFQDA | | |
| | | QLKSANTGNNERIINESIKKLKRKPPSTNAGRGQKDRLTCP | | |
| | | SCDSYEKKPPKEFLERFKSLLQKMIHQHLSSRTHGSEDS | | |
| IL-21_5272 | K81D, V98E, | MRSSPGNMERIVICLMVIFLGTLVHKSSSQGQDRHMIRMRQ | 0.519 | 892 |
| | R115D, T121E | LIDIVDQLKNYVNDLVPEFLPAPEDVETNCEWSAFSCFQDA | | |
| | | QLKSANTGNNERIINESIKKLKRKPPSTNAGRDQKHRLECP | | |
| | | SCDSYEKKPPKEFLERFKSLLQKMIHQHLSSRTHGSEDS | | |
| IL-21_5304 | K81D, V98E, | MRSSPGNMERIVICLMVIFLGTLVHKSSSQGQDRHMIRMRQ | 0.521 | 893 |
| | P107V, R115D | LIDIVDQLKNYVNDLVPEFLPAPEDVETNCEWSAFSCFQDA | | |
| | | QLKSANTGNNERIINESIKKLKRKVPSTNAGRDQKHRLTCP | | |
| | | SCDSYEKKPPKEFLERFKSLLQKMIHQHLSSRTHGSEDS | | |
| IL-21_6018 | H35F, K81D, | MRSSPGNMERIVICLMVIFLGTLVHKSSSQGQDREMIRMRQ | 0.533 | 894 |
| | V98E, T110D | LIDIVDQLKNYVNDLVPEFLPAPEDVETNCEWSAFSCFQDA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5305 | K81D, V98E, | MRSSPGNMERIVICLMVIFLGTLVHKSSSQGQDRHMIRMRQ | 0.534 | 895 |
| | 1P07V, R114G | LIDIVDQLKNYVNDLVPEFLPAPEDVETNCEWSAFSCFQDA | | |
| | | QLKSANTGNNERIINESIKKLKRKVPSTNAGGRQKHRLTCP | | |
| | | SCDSYEKKPPKEFLERFKSLLQKMIHQHLSSRTHGSEDS | | |
| IL-21_5976 | H35F, V98E, | MRSSPGNMERIVICLMVIFLGTLVHKSSSQGQDRFMIRMRQ | 0.542 | 896 |
| | R114G, R115D | LIDIVDQLKNYVNDLVPEFLPAPEDVETNCEWSAFSCFQKA | | |
| | | QLKSANTGNNERIINESIKKLKRKPPSTNAGGDQKHRLTCP | | |
| | | SCDSYEKKPPKEFLERFKSLLQKMIHQHLSSRTHGSEDS | | |
| IL-21_6016 | H35F, K81D, | MRSSPGNMERIVICLMVIFLGTLVHKSSSQGQDREMIRMRQ | 0.543 | 897 |
| | V98E, R114Q | LIDIVDQLKNYVNDLVPEFLPAPEDVETNCEWSAFSCFQDA | | |
| | | QLKSANTGNNERIINESIKKLKRKPPSTNAGORQKHRLTCP | | |
| | | SCDSYEKKPPKEFLERFKSLLQKMIHQHLSSRTHGSEDS | | |
| IL-21_5072 | V98E, R114G, | MRSSPGNMERIVICLMVIFLGTLVHKSSSQGQDRHMIRMRQ | 0.545 | 898 |
| | R115D, R119T | LIDIVDQLKNYVNDLVPEFLPAPEDVETNCEWSAFSCFQKA | | |
| | | QLKSANTGNNERIINESIKKLKRKPPSTNAGGDQKHTLTCP | | |
| | | SCDSYEKKPPKEFLERFKSLLOKMIHQHLSSRTHGSEDS | | |
| IL-21_5107 | V98E, T110D, | MRSSPGNMERIVICLMVIFLGTLVHKSSSQGQDRHMIRMRQ | 0.547 | 899 |
| | R115G, H118D | LIDIVDQLKNYVNDLVPEFLPAPEDVETNCEWSAFSCFQKA | | |
| | | QLKSANTGNNERIINESIKKLKRKPPSDNAGRGQKDRLTCP | | |
| | | SCDSYEKKPPKEFLERFKSLLQKMIHQHLSSRTHGSEDS | | |
| IL-21_5508 | N70G, K81D, | MRSSPGNMERIVICLMVIFLGTLVHKSSSQGQDRHMIRMRQ | 0.552 | 900 |
| | V98E, P107V | LIDIVDQLKNYVNDLVPEFLPAPEDVETGCEWSAFSCFQDA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5283 | K81D, V98E, | MRSSPGNMERIVICLMVIFLGTLVHKSSSQGQDRHMIRMRQ | 0.554 | 901 |
| | R114Q, R115G | LIDIVDQLKNYVNDLVPEFLPAPEDVETNCEWSAFSCFQDA | | |
| | | QLKSANTGNNERIINESIKKLKRKPPSTNAGQGQKHRLTCP | | |
| | | SCDSYEKKPPKEFLERFKSLLQKMIHQHLSSRTHGSEDS | | |
| IL-21_5308 | K81D, V98E, | MRSSPGNMERIVICLMVIFLGTLVHKSSSQGQDRHMIRMRQ | 0.561 | 902 |
| | P107V, T110D | LIDIVDQLKNYVNDLVPEFLPAPEDVETNCEWSAFSCFQDA | | |
| | | QLKSANTGNNERIINESIKKLKRKVPSDNAGRRQKHRLTCP | | |
| | | SCDSYEKKPPKEFLERFKSLLQKMIHQHLSSRTHGSEDS | | |
| IL-21_5296 | K81D, V98E, | MRSSPGNMERIVICLMVIFLGTLVHKSSSQGQDRHMIRMRQ | 0.562 | 903 |
| | T110D, R115D | LIDIVDQLKNYVNDLVPEFLPAPEDVETNCEWSAFSCFQDA | | |
| | | QLKSANTGNNERIINESIKKLKRKPPSDNAGRDQKHRLTCP | | |
| | | SCDSYEKKPPKEFLERFKSLLQKMIHQHLSSRTHGSEDS | | |
| IL-21_5267 | K81D, V98E, | MRSSPGNMERIVICLMVIFLGTLVHKSSSQGQDRHMIRMRQ | 0.566 | 904 |
| | H118D, T121E | LIDIVDQLKNYVNDLVPEFLPAPEDVETNCEWSAFSCFQDA | | |
| | | QLKSANTGNNERIINESIKKLKRKPPSTNAGRROKDRLECP | | |
| | | SCDSYEKKPPKEFLERFKSLLQKMIHQHLSSRTHGSEDS | | |
| IL-21_5120 | V98E, T110D, | MRSSPGNMERIVICLMVIFLGTLVHKSSSQGQDRHMIRMRQ | 0.571 | 905 |
| | R114Q, R115D | LIDIVDQLKNYVNDLVPEFLPAPEDVETNCEWSAFSCFQKA | | |
| | | QLKSANTGNNERIINESIKKLKRKPPSDNAGQDQKHRLTCP | | |
| | | SCDSYEKKPPKEFLERFKSLLQKMIHQHLSSRTHGSEDS | | |
| IL-21_6004 | H35F, K81D, | MRSSPGNMERIVICLMVIFLGTLVHKSSSQGQDREMIRMRQ | 0.573 | 906 |
| | T110D, R114G | LIDIVDQLKNYVNDLVPEFLPAPEDVETNCEWSAFSCFQDA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5302 | K81D, V98E, | MRSSPGNMERIVICLMVIFLGTLVHKSSSQGQDRHMIRMRQ | 0.576 | 907 |
| | P107V, H118D | LIDIVDQLKNYVNDLVPEFLPAPEDVETNCEWSAFSCFQDA | | |
| | | QLKSANTGNNERIINESIKKLKRKVPSTNAGRRQKDRLTCP | | |
| | | SCDSYEKKPPKEFLERFKSLLQKMIHQHLSSRTHGSEDS | | |
| IL-21_5290 | K81D, V98E, | MRSSPGNMERIVICLMVIFLGTLVHKSSSQGQDRHMIRMRQ | 0.579 | 908 |
| | A112P, R114G | LIDIVDQLKNYVNDLVPEFLPAPEDVETNCEWSAFSCFQDA | | |
| | | QLKSANTGNNERIINESIKKLKRKPPSTNPGGRQKHRLTCP | | |
| | | SCDSYEKKPPKEFLERFKSLLQKMIHQHLSSRTHGSEDS | | |
| IL-21_5239 | K81D, P107V, | MRSSPGNMERIVICLMVIFLGTLVHKSSSQGQDRHMIRMRQ | 0.581 | 909 |
| | R115D, R119T | LIDIVDQLKNYVNDLVPEFLPAPEDVETNCEWSAFSCFQDA | | |
| | | QLKSANTGNNERIINVSIKKLKRKVPSTNAGRDQKHTLTCP | | |
| | | SCDSYEKKPPKEFLERFKSLLQKMIHQHLSSRTHGSEDS | | |
| IL-21_5668 | K81D, R114G, | MRSSPGNMERIVICLMVIFLGTLVHKSSSQGQDRHMIRMRQ | 0.583 | 910 |
| | R115D | LIDIVDQLKNYVNDLVPEFLPAPEDVETNCEWSAFSCFQDA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5082 | V98E, R114Q, | MRSSPGNMERIVICLMVIFLGTLVHKSSSQGQDRHMIRMRQ | 0.584 | 911 |
| | R115D, H118D | LIDIVDQLKNYVNDLVPEFLPAPEDVETNCEWSAFSCFQKA | | |
| | | QLKSANTGNNERIINESIKKLKRKPPSTNAGQDQKDRLTCP | | |
| | | SCDSYEKKPPKEFLERFKSLLQKMIHQHLSSRTHGSEDS | | |
| IL-21_5146 | V98E, P107V, | MRSSPGNMERIVICLMVIFLGTLVHKSSSQGQDRHMIRMRQ | 0.586 | 912 |
| | R114Q, R115D | LIDIVDQLKNYVNDLVPEFLPAPEDVETNCEWSAFSCFQKA | | |
| | | QLKSANTGNNERIINESIKKLKRKVPSTNAGQDQKHRLTCP | | |
| | | SCDSYEKKPPKEFLERFKSLLQKMIHQHLSSRTHGSEDS | | |
| IL-21_5073 | V98E, R114G, | MRSSPGNMERIVICLMVIFLGTLVHKSSSQGQDRHMIRMRQ | 0.591 | 913 |
| | R115D, H118D | LIDIVDQLKNYVNDLVPEFLPAPEDVETNCEWSAFSCFQKA | | |
| | | QLKSANTGNNERIINESIKKLKRKPPSTNAGGDQKDRLTCP | | |
| | | SCDSYEKKPPKEFLERFKSLLQKMIHQHLSSRTHGSEDS | | |
| IL-21_6165 | H35I, K81D, | MRSSPGNMERIVICLMVIFLGTLVHKSSSQGQDRIMIRMRQ | 0.595 | 914 |
| | V98E, T110D | LIDIVDQLKNYVNDLVPEFLPAPEDVETNCEWSAFSCFQDA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5443 | N70G, V98E, | MRSSPGNMERIVICLMVIFLGTLVHKSSSQGQDRHMIRMRQ | 0.595 | 915 |
| | T110D, R115D | LIDIVDQLKNYVNDLVPEFLPAPEDVETGCEWSAFSCFQKA | | |
| | | QLKSANTGNNERIINESIKKLKRKPPSDNAGRDQKHRLTCP | | |
| | | SCDSYEKKPPKEFLERFKSLLQKMIHQHLSSRTHGSEDS | | |
| IL-21_5306 | K81D, V98E, | MRSSPGNMERIVICLMVIFLGTLVHKSSSQGQDRHMIRMRQ | 0.6 | 916 |
| | P107V, R114Q | LIDIVDQLKNYVNDLVPEFLPAPEDVETNCEWSAFSCFQDA | | |
| | | QLKSANTGNNERIINESIKKLKRKVPSTNAGORQKHRLTCP | | |
| | | SCDSYEKKPPKEFLERFKSLLQKMIHQHLSSRTHGSEDS | | |
| IL-21_6125 | H35I, V98E, | MRSSPGNMERIVICLMVIFLGTLVHKSSSQGQDRIMIRMRQ | 0.603 | 917 |
| | R114Q, R115D | LIDIVDQLKNYVNDLVPEFLPAPEDVETNCEWSAFSCFQKA | | |
| | | QLKSANTGNNERIINESIKKLKRKPPSTNAGQDQKHRLTCP | | |
| | | SCDSYEKKPPKEFLERFKSLLQKMIHQHLSSRTHGSEDS | | |
| IL-21_6186 | H35I, N70G, | MRSSPGNMERIVICLMVIFLGTLVHKSSSQGQDRIMIRMRQ | 0.606 | 918 |
| | V98E, R115D | LIDIVDQLKNYVNDLVPEFLPAPEDVETGCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_6017 | H35F, K81D, | MRSSPGNMERIVICLMVIFLGTLVHKSSSQGQDREMIRMRQ | 0.606 | 919 |
| | V98E, A112P | LIDIVDQLKNYVNDLVPEFLPAPEDVETNCEWSAFSCFQDA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5250 | K81D, P107V, | MRSSPGNMERIVICLMVIFLGTLVHKSSSQGQDRHMIRMRQ | 0.609 | 920 |
| | R114Q, R115D | LIDIVDQLKNYVNDLVPEFLPAPEDVETNCEWSAFSCFQDA | | |
| | | QLKSANTGNNERIINVSIKKLKRKVPSTNAGQDQKHRLTCP | | |
| | | SCDSYEKKPPKEFLERFKSLLQKMIHQHLSSRTHGSEDS | | |
| IL-21_6166 | H35I, K81D, | MRSSPGNMERIVICLMVIFLGTLVHKSSSQGQDRIMIRMRQ | 0.611 | 921 |
| | V98E, P107V | LIDIVDQLKNYVNDLVPEFLPAPEDVETNCEWSAFSCFQDA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5224 | K81D, T110D, | MRSSPGNMERIVICLMVIFLGTLVHKSSSQGQDRHMIRMRQ | 0.611 | 922 |
| | R114Q, R115D | LIDIVDQLKNYVNDLVPEFLPAPEDVETNCEWSAFSCFQDA | | |
| | | QLKSANTGNNERIINVSIKKLKRKPPSDNAGQDQKHRLTCP | | |
| | | SCDSYEKKPPKEFLERFKSLLQKMIHQHLSSRTHGSEDS | | |
| IL-21_5702 | K81D, V98E, | MRSSPGNMERIVICLMVIFLGTLVHKSSSQGQDRHMIRMRQ | 0.611 | 923 |
| | R115D | LIDIVDQLKNYVNDLVPEFLPAPEDVETNCEWSAFSCFQDA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5625 | V98E, R114G, | MRSSPGNMERIVICLMVIFLGTLVHKSSSQGQDRHMIRMRQ | 0.611 | 924 |
| | R115D | LIDIVDQLKNYVNDLVPEFLPAPEDVETNCEWSAFSCFQKA | | |
| | | QLKSANTGNNERIINESIKKLKRKPPSTNAGGDQKHRLTCP | | |
| | | SCDSYEKKPPKEFLERFKSLLQKMIHQHLSSRTHGSEDS | | |
| IL-21_5706 | K81D, V98E, | MRSSPGNMERIVICLMVIFLGTLVHKSSSQGQDRHMIRMRQ | 0.613 | 925 |
| | T110D | LIDIVDQLKNYVNDLVPEFLPAPEDVETNCEWSAFSCFQDA | | |
| | | QLKSANTGNNERIINESIKKLKRKPPSDNAGRROKHRLTCP | | |
| | | SCDSYEKKPPKEFLERFKSLLOKMIHQHLSSRTHGSEDS | | |
| IL-21_6161 | H35I, K81D, | MRSSPGNMERIVICLMVIFLGTLVHKSSSQGQDRIMIRMRQ | 0.613 | 926 |
| | V98E, R115D | LIDIVDQLKNYVNDLVPEFLPAPEDVETNCEWSAFSCFQDA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5110 | V98E, T110D, | MRSSPGNMERIVICLMVIFLGTLVHKSSSQGQDRHMIRMRQ | 0.614 | 927 |
| | R115D, H118D | LIDIVDQLKNYVNDLVPEFLPAPEDVETNCEWSAFSCFQKA | | |
| | | QLKSANTGNNERIINESIKKLKRKPPSDNAGRDQKDRLTCP | | |
| | | SCDSYEKKPPKEFLERFKSLLQKMIHQHLSSRTHGSEDS | | |
| IL-21_5249 | K81D, P107V, | MRSSPGNMERIVICLMVIFLGTLVHKSSSQGQDRHMIRMRQ | 0.617 | 928 |
| | R114Q, R115G | LIDIVDQLKNYVNDLVPEFLPAPEDVETNCEWSAFSCFQDA | | |
| | | QLKSANTGNNERIINVSIKKLKRKVPSTNAGQGQKHRLTCP | | |
| | | SCDSYEKKPPKEFLERFKSLLQKMIHQHLSSRTHGSEDS | | |
| IL-21_5167 | K81D, R115D, | MRSSPGNMERIVICLMVIFLGTLVHKSSSQGQDRHMIRMRQ | 0.617 | 929 |
| | H118D, T121E | LIDIVDQLKNYVNDLVPEFLPAPEDVETNCEWSAFSCFQDA | | |
| | | QLKSANTGNNERIINVSIKKLKRKPPSTNAGRDQKDRLECP | | |
| | | SCDSYEKKPPKEFLERFKSLLQKMIHQHLSSRTHGSEDS | | |
| IL-21_5997 | H35F, K81D, | MRSSPGNMERIVICLMVIFLGTLVHKSSSQGQDREMIRMRQ | 0.619 | 930 |
| | R114Q, R115D | LIDIVDQLKNYVNDLVPEFLPAPEDVETNCEWSAFSCFQDA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_6014 | H35F, K81D, | MRSSPGNMERIVICLMVIFLGTLVHKSSSQGQDREMIRMRQ | 0.62 | 931 |
| | V98E, R115D | LIDIVDQLKNYVNDLVPEFLPAPEDVETNCEWSAFSCFQDA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5431 | N70G, V98E, | MRSSPGNMERIVICLMVIFLGTLVHKSSSQGQDRHMIRMRQ | 0.621 | 932 |
| | R114Q, R115D | LIDIVDQLKNYVNDLVPEFLPAPEDVETGCEWSAFSCFQKA | | |
| | | QLKSANTGNNERIINESIKKLKRKPPSTNAGQDQKHRLTCP | | |
| | | SCDSYEKKPPKEFLERFKSLLQKMIHQHLSSRTHGSEDS | | |
| IL-21_5707 | K81D, V98E, | MRSSPGNMERIVICLMVIFLGTLVHKSSSQGQDRHMIRMRQ | 0.622 | 933 |
| | P107V | LIDIVDQLKNYVNDLVPEFLPAPEDVETNCEWSAFSCFQDA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5978 | H35F, V98E, | MRSSPGNMERIVICLMVIFLGTLVHKSSSQGQDRFMIRMRQ | 0.623 | 934 |
| | R114Q, R115D | LIDIVDQLKNYVNDLVPEFLPAPEDVETNCEWSAFSCFQKA | | |
| | | QLKSANTGNNERIINESIKKLKRKPPSTNAGQDQKHRLTCP | | |
| | | SCDSYEKKPPKEFLERFKSLLQKMIHQHLSSRTHGSEDS | | |
| IL-21_6163 | H35I, K81D, | MRSSPGNMERIVICLMVIFLGTLVHKSSSQGQDRIMIRMRQ | 0.624 | 935 |
| | V98E, R114Q | LIDIVDQLKNYVNDLVPEFLPAPEDVETNCEWSAFSCFQDA | | |
| | | QLKSANTGNNERIINESIKKLKRKPPSTNAGQRQKHRLTCP | | |
| | | SCDSYEKKPPKEFLERFKSLLOKMIHQHLSSRTHGSEDS | | |
| IL-21_5079 | V98E, R114Q, | MRSSPGNMERIVICLMVIFLGTLVHKSSSQGQDRHMIRMRQ | 0.626 | 936 |
| | R115G, H118D | LIDIVDQLKNYVNDLVPEFLPAPEDVETNCEWSAFSCFQKA | | |
| | | QLKSANTGNNERIINESIKKLKRKPPSTNAGQGQKDRLTCP | | |
| | | SCDSYEKKPPKEFLERFKSLLQKMIHQHLSSRTHGSEDS | | |
| IL-21_5113 | V98E, T110D, | MRSSPGNMERIVICLMVIFLGTLVHKSSSQGQDRHMIRMRQ | 0.627 | 937 |
| | R114G, H118D | LIDIVDQLKNYVNDLVPEFLPAPEDVETNCEWSAFSCFQKA | | |
| | | QLKSANTGNNERIINESIKKLKRKPPSDNAGGRQKDRLTCP | | |
| | | SCDSYEKKPPKEFLERFKSLLQKMIHQHLSSRTHGSEDS | | |
| IL-21_5299 | K81D, V98E, | MRSSPGNMERIVICLMVIFLGTLVHKSSSQGQDRHMIRMRQ | 0.628 | 938 |
| | T110D, A112P | LIDIVDQLKNYVNDLVPEFLPAPEDVETNCEWSAFSCFQDA | | |
| | | QLKSANTGNNERIINESIKKLKRKPPSDNPGRRQKHRLTCP | | |
| | | SCDSYEKKPPKEFLERFKSLLQKMIHQHLSSRTHGSEDS | | |
| IL-21_5307 | K81D, V98E, | MRSSPGNMERIVICLMVIFLGTLVHKSSSQGQDRHMIRMRQ | 0.631 | 939 |
| | P107V, A112P | LIDIVDQLKNYVNDLVPEFLPAPEDVETNCEWSAFSCFQDA | | |
| | | QLKSANTGNNERIINESIKKLKRKVPSTNPGRROKHRLTCP | | |
| | | SCDSYEKKPPKEFLERFKSLLQKMIHQHLSSRTHGSEDS | | |
| IL-21_5104 | V98E, T110D, | MRSSPGNMERIVICLMVIFLGTLVHKSSSQGQDRHMIRMRQ | 0.632 | 940 |
| | H118D, R119T | LIDIVDQLKNYVNDLVPEFLPAPEDVETNCEWSAFSCFQKA | | |
| | | QLKSANTGNNERIINESIKKLKRKPPSDNAGRROKDTLTCP | | |
| | | SCDSYEKKPPKEFLERFKSLLQKMIHQHLSSRTHGSEDS | | |
| IL-21_5279 | K81D, V98E, | MRSSPGNMERIVICLMVIFLGTLVHKSSSQGQDRHMIRMRQ | 0.634 | 941 |
| | R114G, R115D | LIDIVDQLKNYVNDLVPEFLPAPEDVETNCEWSAFSCFQDA | | |
| | | QLKSANTGNNERIINESIKKLKRKPPSTNAGGDQKHRLTCP | | |
| | | SCDSYEKKPPKEFLERFKSLLQKMIHQHLSSRTHGSEDS | | |
| IL-21_5064 | V98E, R115D, | MRSSPGNMERIVICLMVIFLGTLVHKSSSQGQDRHMIRMRQ | 0.634 | 942 |
| | H118D, R119T | LIDIVDQLKNYVNDLVPEFLPAPEDVETNCEWSAFSCFQKA | | |
| | | QLKSANTGNNERIINESIKKLKRKPPSTNAGRDQKDTLTCP | | |
| | | SCDSYEKKPPKEFLERFKSLLQKMIHQHLSSRTHGSEDS | | |
| IL-21_5115 | V98E, T110D, | MRSSPGNMERIVICLMVIFLGTLVHKSSSQGQDRHMIRMRQ | 0.635 | 943 |
| | R114G, R115D | LIDIVDQLKNYVNDLVPEFLPAPEDVETNCEWSAFSCFQKA | | |
| | | QLKSANTGNNERIINESIKKLKRKPPSDNAGGDQKHRLTCP | | |
| | | SCDSYEKKPPKEFLERFKSLLQKMIHQHLSSRTHGSEDS | | |
| IL-21_5642 | V98E, T110D, | MRSSPGNMERIVICLMVIFLGTLVHKSSSQGQDRHMIRMRQ | 0.635 | 944 |
| | R115D | LIDIVDQLKNYVNDLVPEFLPAPEDVETNCEWSAFSCFQKA | | |
| | | QLKSANTGNNERIINESIKKLKRKPPSDNAGRDQKHRLTCP | | |
| | | SCDSYEKKPPKEFLERFKSLLQKMIHQHLSSRTHGSEDS | | |
| IL-21_6015 | H35F, K81D, | MRSSPGNMERIVICLMVIFLGTLVHKSSSQGQDREMIRMRQ | 0.637 | 945 |
| | V98E, R114G | LIDIVDQLKNYVNDLVPEFLPAPEDVETNCEWSAFSCFQDA | | |
| | | QLKSANTGNNERIINESIKKLKRKPPSTNAGGROKHRLTCP | | |
| | | SCDSYEKKPPKEFLERFKSLLQKMIHQHLSSRTHGSEDS | | |
| IL-21_5421 | N70G, V98E, | MRSSPGNMERIVICLMVIFLGTLVHKSSSQGQDRHMIRMRQ | 0.638 | 946 |
| | R115D, H118D | LIDIVDQLKNYVNDLVPEFLPAPEDVETGCEWSAFSCFQKA | | |
| | | QLKSANTGNNERIINESIKKLKRKPPSTNAGRDQKDRLTCP | | |
| | | SCDSYEKKPPKEFLERFKSLLQKMIHQHLSSRTHGSEDS | | |
| IL-21_6008 | H35F, K81D, | MRSSPGNMERIVICLMVIFLGTLVHKSSSQGQDRFMIRMRQ | 0.639 | 947 |
| | P107V, R115D | LIDIVDQLKNYVNDLVPEFLPAPEDVETNCEWSAFSCFQDA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5298 | K81D, V98E, | MRSSPGNMERIVICLMVIFLGTLVHKSSSQGQDRHMIRMRQ | 0.64 | 948 |
| | T110D, R114Q | LIDIVDQLKNYVNDLVPEFLPAPEDVETNCEWSAFSCFQDA | | |
| | | QLKSANTGNNERIINESIKKLKRKPPSDNAGORQKHRLTCP | | |
| | | SCDSYEKKPPKEFLERFKSLLQKMIHQHLSSRTHGSEDS | | |
| IL-21_5486 | N70G, K81D, | MRSSPGNMERIVICLMVIFLGTLVHKSSSQGQDRHMIRMRQ | 0.64 | 949 |
| | T110D, R115D | LIDIVDQLKNYVNDLVPEFLPAPEDVETGCEWSAFSCFQDA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6318 | H35F, K81D, | MRSSPGNMERIVICLMVIFLGTLVHKSSSQGQDREMIRMRQ | 0.64 | 950 |
| | V98E | LIDIVDQLKNYVNDLVPEFLPAPEDVETNCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5273 | K81D, V98E, | MRSSPGNMERIVICLMVIFLGTLVHKSSSQGQDRHMIRMRQ | 0.643 | 95 |
| | R115D, R119T | LIDIVDQLKNYVNDLVPEFLPAPEDVETNCEWSAFSCFQDA | | |
| | | QLKSANTGNNERIINESIKKLKRKPPSTNAGRDQKHTLTCP | | |
| | | SCDSYEKKPPKEFLERFKSLLQKMIHQHLSSRTHGSEDS | | |
| IL-21_6002 | H35F, K81D, | MRSSPGNMERIVICLMVIFLGTLVHKSSSQGQDREMIRMRQ | 0.643 | 952 |
| | T110D, R115G | LIDIVDQLKNYVNDLVPEFLPAPEDVETNCEWSAFSCFQDA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_5260 | K81D, P107V, | MRSSPGNMERIVICLMVIFLGTLVHKSSSQGQDRHMIRMRQ | 0.644 | 953 |
| | T110D, H118D | LIDIVDQLKNYVNDLVPEFLPAPEDVETNCEWSAFSCFQDA | | |
| | | QLKSANTGNNERIINVSIKKLKRKVPSDNAGRROKDRLTCP | | |
| | | SCDSYEKKPPKEFLERFKSLLQKMIHQHLSSRTHGSEDS | | |
| IL-21_5292 | K81D, V98E, | MRSSPGNMERIVICLMVIFLGTLVHKSSSQGQDRHMIRMRQ | 0.645 | 954 |
| | T110D, T121E | LIDIVDQLKNYVNDLVPEFLPAPEDVETNCEWSAFSCFQDA | | |
| | | QLKSANTGNNERIINESIKKLKRKPPSDNAGRROKHRLECP | | |
| | | SCDSYEKKPPKEFLERFKSLLQKMIHQHLSSRTHGSEDS | | |
| IL-21_6160 | H35I, K81D, | MRSSPGNMERIVICLMVIFLGTLVHKSSSQGQDRIMIRMRQ | 0.645 | 955 |
| | V98E, R115G | LIDIVDQLKNYVNDLVPEFLPAPEDVETNCEWSAFSCFQDA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_5101 | V98E, A112P, | MRSSPGNMERIVICLMVIFLGTLVHKSSSQGQDRHMIRMRQ | 0.647 | 956 |
| | R114Q, R115D | LIDIVDQLKNYVNDLVPEFLPAPEDVETNCEWSAFSCFQKA | | |
| | | QLKSANTGNNERIINESIKKLKRKPPSTNPGQDQKHRLTCP | | |
| | | SCDSYEKKPPKEFLERFKSLLQKMIHQHLSSRTHGSEDS | | |
| IL-21_5281 | K81D, V98E, | MRSSPGNMERIVICLMVIFLGTLVHKSSSQGQDRHMIRMRQ | 0.647 | 957 |
| | R114Q, R119T | LIDIVDQLKNYVNDLVPEFLPAPEDVETNCEWSAFSCFQDA | | |
| | | QLKSANTGNNERIINESIKKLKRKPPSTNAGORQKHTLTCP | | |
| | | SCDSYEKKPPKEFLERFKSLLQKMIHQHLSSRTHGSEDS | | |
| IL-21_5999 | H35F, K81D, | MRSSPGNMERIVICLMVIFLGTLVHKSSSQGQDRFMIRMRQ | 0.648 | 958 |
| | A112P, R115D | LIDIVDQLKNYVNDLVPEFLPAPEDVETNCEWSAFSCFQDA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5158 | V98E, P107V, | MRSSPGNMERIVICLMVIFLGTLVHKSSSQGQDRHMIRMRQ | 0.648 | 959 |
| | T110D, R115D | LIDIVDQLKNYVNDLVPEFLPAPEDVETNCEWSAFSCFQKA | | |
| | | QLKSANTGNNERIINESIKKLKRKVPSDNAGRDQKHRLTCP | | |
| | | SCDSYEKKPPKEFLERFKSLLQKMIHQHLSSRTHGSEDS | | |
| IL-21_5274 | K81D, V98E, | MRSSPGNMERIVICLMVIFLGTLVHKSSSQGQDRHMIRMRQ | 0.65 | 960 |
| | R115D, H118D | LIDIVDQLKNYVNDLVPEFLPAPEDVETNCEWSAFSCFQDA | | |
| | | QLKSANTGNNERIINESIKKLKRKPPSTNAGRDQKDRLTCP | | |
| | | SCDSYEKKPPKEFLERFKSLLQKMIHQHLSSRTHGSEDS | | |
| IL-21_5487 | N70G, K81D, | MRSSPGNMERIVICLMVIFLGTLVHKSSSQGQDRHMIRMRQ | 0.651 | 96 |
| | T110D, R114G | LIDIVDQLKNYVNDLVPEFLPAPEDVETGCEWSAFSCFQDA | | |
| | | QLKSANTGNNERIINVSIKKLKRKPPSDNAGGROKHRLTCP | | |
| | | SCDSYEKKPPKEFLERFKSLLQKMIHQHLSSRTHGSEDS | | |
| IL-21_5181 | K81D, R114Q, | MRSSPGNMERIVICLMVIFLGTLVHKSSSQGQDRHMIRMRQ | 0.651 | 962 |
| | R115G, T121E | LIDIVDQLKNYVNDLVPEFLPAPEDVETNCEWSAFSCFQDA | | |
| | | QLKSANTGNNERIINVSIKKLKRKPPSTNAGQGQKHRLECP | | |
| | | SCDSYEKKPPKEFLERFKSLLQKMIHQHLSSRTHGSEDS | | |
| IL-21_6197 | H35I, N70G, | MRSSPGNMERIVICLMVIFLGTLVHKSSSQGQDRIMIRMRQ | 0.651 | 963 |
| | K81D, T110D | LIDIVDQLKNYVNDLVPEFLPAPEDVETGCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5474 | N70G, K81D, | MRSSPGNMERIVICLMVIFLGTLVHKSSSQGQDRHMIRMRQ | 0.652 | 964 |
| | R114Q, R115D | LIDIVDQLKNYVNDLVPEFLPAPEDVETGCEWSAFSCFQDA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_6123 | H35I, V98E, | MRSSPGNMERIVICLMVIFLGTLVHKSSSQGQDRIMIRMRQ | 0.655 | 965 |
| | R114G, R115D | LIDIVDQLKNYVNDLVPEFLPAPEDVETNCEWSAFSCFQKA | | |
| | | QLKSANTGNNERIINESIKKLKRKPPSTNAGGDQKHRLTCP | | |
| | | SCDSYEKKPPKEFLERFKSLLQKMIHQHLSSRTHGSEDS | | |
| IL-21_5996 | H35F, K81D, | MRSSPGNMERIVICLMVIFLGTLVHKSSSQGQDREMIRMRQ | 0.656 | 966 |
| | R114Q, R115G | LIDIVDQLKNYVNDLVPEFLPAPEDVETNCEWSAFSCFQDA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5980 | H35F, V98E, | MRSSPGNMERIVICLMVIFLGTLVHKSSSQGQDREMIRMRQ | 0.656 | 967 |
| | A112P, R115D | LIDIVDQLKNYVNDLVPEFLPAPEDVETNCEWSAFSCFQKA | | |
| | | QLKSANTGNNERIINESIKKLKRKPPSTNPGRDQKHRLTCP | | |
| | | SCDSYEKKPPKEFLERFKSLLQKMIHQHLSSRTHGSEDS | | |
| IL-21_6358 | H35I, V98E, | MRSSPGNMERIVICLMVIFLGTLVHKSSSQGQDRIMIRMRQ | 0.657 | 968 |
| | R115D | LIDIVDQLKNYVNDLVPEFLPAPEDVETNCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5288 | K81D, V98E, | MRSSPGNMERIVICLMVIFLGTLVHKSSSQGQDRHMIRMRQ | 0.661 | 969 |
| | A112P, R115G | LIDIVDQLKNYVNDLVPEFLPAPEDVETNCEWSAFSCFQDA | | |
| | | QLKSANTGNNERIINESIKKLKRKPPSTNPGRGQKHRLTCP | | |
| | | SCDSYEKKPPKEFLERFKSLLQKMIHQHLSSRTHGSEDS | | |
| IL-21_5130 | V98E, P107V, | MRSSPGNMERIVICLMVIFLGTLVHKSSSQGQDRHMIRMRQ | 0.663 | 970 |
| | H118D, R119T | LIDIVDQLKNYVNDLVPEFLPAPEDVETNCEWSAFSCFQKA | | |
| | | QLKSANTGNNERIINESIKKLKRKVPSTNAGRRQKDTLTCP | | |
| | | SCDSYEKKPPKEFLERFKSLLQKMIHQHLSSRTHGSEDS | | |
| IL-21_5200 | K81D, A112P, | MRSSPGNMERIVICLMVIFLGTLVHKSSSQGQDRHMIRMRQ | 0.663 | 971 |
| | R114G, R115D | LIDIVDQLKNYVNDLVPEFLPAPEDVETNCEWSAFSCFQDA | | |
| | | QLKSANTGNNERIINVSIKKLKRKPPSTNPGGDQKHRLTCP | | |
| | | SCDSYEKKPPKEFLERFKSLLQKMIHQHLSSRTHGSEDS | | |
| IL-21_5300 | K81D, V98E, | MRSSPGNMERIVICLMVIFLGTLVHKSSSQGQDRHMIRMRQ | 0.663 | 972 |
| | P107V, T121E | LIDIVDQLKNYVNDLVPEFLPAPEDVETNCEWSAFSCFQDA | | |
| | | QLKSANTGNNERIINESIKKLKRKVPSTNAGRRQKHRLECP | | |
| | | SCDSYEKKPPKEFLERFKSLLQKMIHQHLSSRTHGSEDS | | |
| IL-21_5295 | K81D, V98E, | MRSSPGNMERIVICLMVIFLGTLVHKSSSQGQDRHMIRMRQ | 0.663 | 973 |
| | T110D, R115G | LIDIVDQLKNYVNDLVPEFLPAPEDVETNCEWSAFSCFQDA | | |
| | | QLKSANTGNNERIINESIKKLKRKPPSDNAGRGQKHRLTCP | | |
| | | SCDSYEKKPPKEFLERFKSLLQKMIHQHLSSRTHGSEDS | | |
| IL-21_5703 | K81D, V98E, | MRSSPGNMERIVICLMVIFLGTLVHKSSSQGQDRHMIRMRQ | 0.665 | 974 |
| | R114G | LIDIVDQLKNYVNDLVPEFLPAPEDVETNCEWSAFSCFQDA | | |
| | | QLKSANTGNNERIINESIKKLKRKPPSTNAGGROKHRLTCP | | |
| | | SCDSYEKKPPKEFLERFKSLLQKMIHQHLSSRTHGSEDS | | |
| IL-21_5426 | N70G, V98E, | MRSSPGNMERIVICLMVIFLGTLVHKSSSQGQDRHMIRMRQ | 0.665 | 975 |
| | R114G, R115D | LIDIVDQLKNYVNDLVPEFLPAPEDVETGCEWSAFSCFQKA | | |
| | | QLKSANTGNNERIINESIKKLKRKPPSTNAGGDQKHRLTCP | | |
| | | SCDSYEKKPPKEFLERFKSLLQKMIHQHLSSRTHGSEDS | | |
| IL-21_5994 | H35F, K81D, | MRSSPGNMERIVICLMVIFLGTLVHKSSSQGQDREMIRMRQ | 0.665 | 976 |
| | R114G, R115G | LIDIVDQLKNYVNDLVPEFLPAPEDVETNCEWSAFSCFQDA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5504 | N70G, K81D, | MRSSPGNMERIVICLMVIFLGTLVHKSSSQGQDRHMIRMRQ | 0.666 | 977 |
| | V98E, R114G | LIDIVDQLKNYVNDLVPEFLPAPEDVETGCEWSAFSCFQDA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_6019 | H35F, K81D, | MRSSPGNMERIVICLMVIFLGTLVHKSSSQGQDREMIRMRQ | 0.668 | 978 |
| | V98E, P107V | LIDIVDQLKNYVNDLVPEFLPAPEDVETNCEWSAFSCFQDA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_6142 | H35I, K81D, | MRSSPGNMERIVICLMVIFLGTLVHKSSSQGQDRIMIRMRQ | 0.674 | 979 |
| | R114G, R115D | LIDIVDQLKNYVNDLVPEFLPAPEDVETNCEWSAFSCFQDA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5143 | V98E, P107V, | MRSSPGNMERIVICLMVIFLGTLVHKSSSQGQDRHMIRMRQ | 0.675 | 980 |
| | R114Q, R119T | LIDIVDQLKNYVNDLVPEFLPAPEDVETNCEWSAFSCFQKA | | |
| | | QLKSANTGNNERIINESIKKLKRKVPSTNAGORQKHTLTCP | | |
| | | SCDSYEKKPPKEFLERFKSLLQKMIHQHLSSRTHGSEDS | | |
| IL-21_5145 | V98E, P107V, | MRSSPGNMERIVICLMVIFLGTLVHKSSSQGQDRHMIRMRQ | 0.677 | 981 |
| | R114Q, R115G | LIDIVDQLKNYVNDLVPEFLPAPEDVETNCEWSAFSCFQKA | | |
| | | QLKSANTGNNERIINESIKKLKRKVPSTNAGQGQKHRLTCP | | |
| | | SCDSYEKKPPKEFLERFKSLLQKMIHQHLSSRTHGSEDS | | |
| IL-21_5472 | N70G, K81D, | MRSSPGNMERIVICLMVIFLGTLVHKSSSQGQDRHMIRMRQ | 0.677 | 982 |
| | R114Q, H118D | LIDIVDQLKNYVNDLVPEFLPAPEDVETGCEWSAFSCFQDA | | |
| | | QLKSANTGNNERIINVSIKKLKRKPPSTNAGQRQKDRLTCP | | |
| | | SCDSYEKKPPKEFLERFKSLLQKMIHQHLSSRTHGSEDS | | |
| IL-21_5280 | K81D, V98E, | MRSSPGNMERIVICLMVIFLGTLVHKSSSQGQDRHMIRMRQ | 0.679 | 983 |
| | R114Q, T121E | LIDIVDQLKNYVNDLVPEFLPAPEDVETNCEWSAFSCFQDA | | |
| | | QLKSANTGNNERIINESIKKLKRKPPSTNAGORQKHRLECP | | |
| | | SCDSYEKKPPKEFLERFKSLLQKMIHQHLSSRTHGSEDS | | |
| IL-21_5144 | V98E, P107V, | MRSSPGNMERIVICLMVIFLGTLVHKSSSQGQDRHMIRMRQ | 0.68 | 984 |
| | R114Q, H118D | LIDIVDQLKNYVNDLVPEFLPAPEDVETNCEWSAFSCFQKA | | |
| | | QLKSANTGNNERIINESIKKLKRKVPSTNAGORQKDRLTCP | | |
| | | SCDSYEKKPPKEFLERFKSLLQKMIHQHLSSRTHGSEDS | | |
| IL-21_5500 | N70G, K81D, | MRSSPGNMERIVICLMVIFLGTLVHKSSSQGQDRHMIRMRQ | 0.68 | 985 |
| | V98E, R119T | LIDIVDQLKNYVNDLVPEFLPAPEDVETGCEWSAFSCFQDA | | |
| | | QLKSANTGNNERIINESIKKLKRKPPSTNAGRRQKHTLTCP | | |
| | | SCDSYEKKPPKEFLERFKSLLQKMIHQHLSSRTHGSEDS | | |
| IL-21_5136 | V98E, P107V, | MRSSPGNMERIVICLMVIFLGTLVHKSSSQGQDRHMIRMRQ | 0.681 | 986 |
| | R115D, H118D | LIDIVDQLKNYVNDLVPEFLPAPEDVETNCEWSAFSCFQKA | | |
| | | QLKSANTGNNERIINESIKKLKRKVPSTNAGRDQKDRLTCP | | |
| | | SCDSYEKKPPKEFLERFKSLLQKMIHQHLSSRTHGSEDS | | |
| IL-21_6132 | H35I, V98E, | MRSSPGNMERIVICLMVIFLGTLVHKSSSQGQDRIMIRMRQ | 0.681 | 987 |
| | T110D, R114G | LIDIVDQLKNYVNDLVPEFLPAPEDVETNCEWSAFSCFQKA | | |
| | | QLKSANTGNNERIINESIKKLKRKPPSDNAGGRQKHRLTCP | | |
| | | SCDSYEKKPPKEFLERFKSLLQKMIHQHLSSRTHGSEDS | | |
| IL-21_5469 | N70G, K81D, | MRSSPGNMERIVICLMVIFLGTLVHKSSSQGQDRHMIRMRQ | 0.681 | 988 |
| | R114G, R115D | LIDIVDQLKNYVNDLVPEFLPAPEDVETGCEWSAFSCFQDA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5704 | K81D, V98E, | MRSSPGNMERIVICLMVIFLGTLVHKSSSQGQDRHMIRMRQ | 0.682 | 989 |
| | R114Q | LIDIVDQLKNYVNDLVPEFLPAPEDVETNCEWSAFSCFQDA | | |
| | | QLKSANTGNNERIINESIKKLKRKPPSTNAGORQKHRLTCP | | |
| | | SCDSYEKKPPKEFLERFKSLLQKMIHQHLSSRTHGSEDS | | |
| IL-21_5135 | V98E, P107V, | MRSSPGNMERIVICLMVIFLGTLVHKSSSQGQDRHMIRMRQ | 0.685 | 990 |
| | R115D, R119T | LIDIVDQLKNYVNDLVPEFLPAPEDVETNCEWSAFSCFQKA | | |
| | | QLKSANTGNNERIINESIKKLKRKVPSTNAGRDQKHTLTCP | | |
| | | SCDSYEKKPPKEFLERFKSLLQKMIHQHLSSRTHGSEDS | | |
| IL-21_5630 | V98E, R114Q, | MRSSPGNMERIVICLMVIFLGTLVHKSSSQGQDRHMIRMRQ | 0.685 | 991 |
| | R115D | LIDIVDQLKNYVNDLVPEFLPAPEDVETNCEWSAFSCFQKA | | |
| | | QLKSANTGNNERIINESIKKLKRKPPSTNAGQDQKHRLTCP | | |
| | | SCDSYEKKPPKEFLERFKSLLQKMIHQHLSSRTHGSEDS | | |
| IL-21_5473 | N70G, K81D, | MRSSPGNMERIVICLMVIFLGTLVHKSSSQGQDRHMIRMRQ | 0.687 | 992 |
| | R114Q, R115G | LIDIVDQLKNYVNDLVPEFLPAPEDVETGCEWSAFSCFQDA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5170 | K81D, R114G, | MRSSPGNMERIVICLMVIFLGTLVHKSSSQGQDRHMIRMRQ | 0.687 | 993 |
| | H118D, T121E | LIDIVDQLKNYVNDLVPEFLPAPEDVETNCEWSAFSCFQDA | | |
| | | QLKSANTGNNERIINVSIKKLKRKPPSTNAGGROKDRLECP | | |
| | | SCDSYEKKPPKEFLERFKSLLQKMIHQHLSSRTHGSEDS | | |
| IL-21_5118 | V98E, T110D, | MRSSPGNMERIVICLMVIFLGTLVHKSSSQGQDRHMIRMRQ | 0.687 | 994 |
| | R114Q, H118D | LIDIVDQLKNYVNDLVPEFLPAPEDVETNCEWSAFSCFQKA | | |
| | | QLKSANTGNNERIINESIKKLKRKPPSDNAGQRQKDRLTCP | | |
| | | SCDSYEKKPPKEFLERFKSLLQKMIHQHLSSRTHGSEDS | | |
| IL-21_6124 | H35I, V98E, | MRSSPGNMERIVICLMVIFLGTLVHKSSSQGQDRIMIRMRQ | 0.688 | 995 |
| | R114Q, R115G | LIDIVDQLKNYVNDLVPEFLPAPEDVETNCEWSAFSCFQKA | | |
| | | QLKSANTGNNERIINESIKKLKRKPPSTNAGQGQKHRLTCP | | |
| | | SCDSYEKKPPKEFLERFKSLLQKMIHQHLSSRTHGSEDS | | |
| IL-21_5834 | K81D, V98E | MRSSPGNMERIVICLMVIFLGTLVHKSSSQGQDRHMIRMRQ | 0.689 | 996 |
| | | LIDIVDQLKNYVNDLVPEFLPAPEDVETNCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5282 | K81D, V98E, | MRSSPGNMERIVICLMVIFLGTLVHKSSSQGQDRHMIRMRQ | 0.691 | 997 |
| | R114Q, H118D | LIDIVDQLKNYVNDLVPEFLPAPEDVETNCEWSAFSCFQDA | | |
| | | QLKSANTGNNERIINESIKKLKRKPPSTNAGORQKDRLTCP | | |
| | | SCDSYEKKPPKEFLERFKSLLQKMIHQHLSSRTHGSEDS | | |
| IL-21_5270 | K81D, V98E, | MRSSPGNMERIVICLMVIFLGTLVHKSSSQGQDRHMIRMRQ | 0.691 | 998 |
| | R115G, R119T | LIDIVDQLKNYVNDLVPEFLPAPEDVETNCEWSAFSCFQDA | | |
| | | QLKSANTGNNERIINESIKKLKRKPPSTNAGRGQKHTLTCP | | |
| | | SCDSYEKKPPKEFLERFKSLLQKMIHQHLSSRTHGSEDS | | |
| IL-21_6190 | H35I, N70G, | MRSSPGNMERIVICLMVIFLGTLVHKSSSQGQDRIMIRMRQ | 0.692 | 999 |
| | V98E, T110D | LIDIVDQLKNYVNDLVPEFLPAPEDVETGCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5166 | K81D, R115D, | MRSSPGNMERIVICLMVIFLGTLVHKSSSQGQDRHMIRMRQ | 0.693 | 1000 |
| | R119T, T121E | LIDIVDQLKNYVNDLVPEFLPAPEDVETNCEWSAFSCFQDA | | |
| | | QLKSANTGNNERIINVSIKKLKRKPPSTNAGRDQKHTLECP | | |
| | | SCDSYEKKPPKEFLERFKSLLQKMIHQHLSSRTHGSEDS | | |
| IL-21_5177 | K81D, R114G, | MRSSPGNMERIVICLMVIFLGTLVHKSSSQGQDRHMIRMRQ | 0.693 | 1001 |
| | R115D, H118D | LIDIVDQLKNYVNDLVPEFLPAPEDVETNCEWSAFSCFQDA | | |
| | | QLKSANTGNNERIINVSIKKLKRKPPSTNAGGDQKDRLTCP | | |
| | | SCDSYEKKPPKEFLERFKSLLQKMIHQHLSSRTHGSEDS | | |
| IL-21_5141 | V98E, P107V, | MRSSPGNMERIVICLMVIFLGTLVHKSSSQGQDRHMIRMRQ | 0.694 | 1002 |
| | R114G, R115D | LIDIVDQLKNYVNDLVPEFLPAPEDVETNCEWSAFSCFQKA | | |
| | | QLKSANTGNNERIINESIKKLKRKVPSTNAGGDQKHRLTCP | | |
| | | SCDSYEKKPPKEFLERFKSLLQKMIHQHLSSRTHGSEDS | | |
| IL-21_5238 | K81D, P107V, | MRSSPGNMERIVICLMVIFLGTLVHKSSSQGQDRHMIRMRQ | 0.695 | 1003 |
| | R115D, T121E | LIDIVDQLKNYVNDLVPEFLPAPEDVETNCEWSAFSCFQDA | | |
| | | QLKSANTGNNERIINVSIKKLKRKVPSTNAGRDQKHRLECP | | |
| | | SCDSYEKKPPKEFLERFKSLLQKMIHQHLSSRTHGSEDS | | |
| IL-21_5212 | K81D, T110D, | MRSSPGNMERIVICLMVIFLGTLVHKSSSQGQDRHMIRMRQ | 0.695 | 1004 |
| | R115D, T121E | LIDIVDQLKNYVNDLVPEFLPAPEDVETNCEWSAFSCFQDA | | |
| | | QLKSANTGNNERIINVSIKKLKRKPPSDNAGRDQKHRLECP | | |
| | | SCDSYEKKPPKEFLERFKSLLQKMIHQHLSSRTHGSEDS | | |
| IL-21_6371 | H35I, K81D, | MRSSPGNMERIVICLMVIFLGTLVHKSSSQGQDRIMIRMRQ | 0.696 | 1005 |
| | V98E | LIDIVDQLKNYVNDLVPEFLPAPEDVETNCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5415 | N70G, V98E, | MRSSPGNMERIVICLMVIFLGTLVHKSSSQGQDRHMIRMRQ | 0.697 | 1006 |
| | H118D, R119T | LIDIVDQLKNYVNDLVPEFLPAPEDVETGCEWSAFSCFQKA | | |
| | | QLKSANTGNNERIINESIKKLKRKPPSTNAGRRQKDTLTCP | | |
| | | SCDSYEKKPPKEFLERFKSLLQKMIHQHLSSRTHGSEDS | | |
| IL-21_5119 | V98E, T110D, | MRSSPGNMERIVICLMVIFLGTLVHKSSSQGQDRHMIRMRQ | 0.697 | 1007 |
| | R114Q, R115G | LIDIVDQLKNYVNDLVPEFLPAPEDVETNCEWSAFSCFQKA | | |
| | | QLKSANTGNNERIINESIKKLKRKPPSDNAGQGQKHRLTCP | | |
| | | SCDSYEKKPPKEFLERFKSLLQKMIHQHLSSRTHGSEDS | | |
| IL-21_5297 | K81D, V98E, | MRSSPGNMERIVICLMVIFLGTLVHKSSSQGQDRHMIRMRQ | 0.697 | 1008 |
| | T110D, R114G | LIDIVDQLKNYVNDLVPEFLPAPEDVETNCEWSAFSCFQDA | | |
| | | QLKSANTGNNERIINESIKKLKRKPPSDNAGGROKHRLTCP | | |
| | | SCDSYEKKPPKEFLERFKSLLQKMIHQHLSSRTHGSEDS | | |
| IL-21_5233 | K81D, P107V, | MRSSPGNMERIVICLMVIFLGTLVHKSSSQGQDRHMIRMRQ | 0.698 | 1009 |
| | H118D, T121E | LIDIVDQLKNYVNDLVPEFLPAPEDVETNCEWSAFSCFQDA | | |
| | | QLKSANTGNNERIINVSIKKLKRKVPSTNAGRRQKDRLECP | | |
| | | SCDSYEKKPPKEFLERFKSLLQKMIHQHLSSRTHGSEDS | | |
| IL-21_5291 | K81D, V98E, | MRSSPGNMERIVICLMVIFLGTLVHKSSSQGQDRHMIRMRQ | 0.698 | 1010 |
| | A112P, R114Q | LIDIVDQLKNYVNDLVPEFLPAPEDVETNCEWSAFSCFQDA | | |
| | | QLKSANTGNNERIINESIKKLKRKPPSTNPGQRQKHRLTCP | | |
| | | SCDSYEKKPPKEFLERFKSLLQKMIHQHLSSRTHGSEDS | | |
| IL-21_5977 | H35F, V98E, | MRSSPGNMERIVICLMVIFLGTLVHKSSSQGQDREMIRMRQ | 0.698 | 1011 |
| | R114Q, R115G | LIDIVDQLKNYVNDLVPEFLPAPEDVETNCEWSAFSCFQKA | | |
| | | QLKSANTGNNERIINESIKKLKRKPPSTNAGQGQKHRLTCP | | |
| | | SCDSYEKKPPKEFLERFKSLLQKMIHQHLSSRTHGSEDS | | |
| IL-21_5462 | N70G, K81D, | MRSSPGNMERIVICLMVIFLGTLVHKSSSQGQDRHMIRMRQ | 0.699 | 1012 |
| | R115D, T121E | LIDIVDQLKNYVNDLVPEFLPAPEDVETGCEWSAFSCFQDA | | |
| | | QLKSANTGNNERIINVSIKKLKRKPPSTNAGRDQKHRLECP | | |
| | | SCDSYEKKPPKEFLERFKSLLQKMIHQHLSSRTHGSEDS | | |
| IL-21_5493 | N70G, K81D, | MRSSPGNMERIVICLMVIFLGTLVHKSSSQGQDRHMIRMRQ | 0.699 | 1013 |
| | P107V, R115G | LIDIVDQLKNYVNDLVPEFLPAPEDVETGCEWSAFSCFQDA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5125 | V98E, T110D, | MRSSPGNMERIVICLMVIFLGTLVHKSSSQGQDRHMIRMRQ | 0.701 | 1014 |
| | A112P, R115D | LIDIVDQLKNYVNDLVPEFLPAPEDVETNCEWSAFSCFQKA | | |
| | | QLKSANTGNNERIINESIKKLKRKPPSDNPGRDQKHRLTCP | | |
| | | SCDSYEKKPPKEFLERFKSLLQKMIHQHLSSRTHGSEDS | | |
| IL-21_6005 | H35F, K81D, | MRSSPGNMERIVICLMVIFLGTLVHKSSSQGQDREMIRMRQ | 0.702 | 1015 |
| | T110D, R114Q | LIDIVDQLKNYVNDLVPEFLPAPEDVETNCEWSAFSCFQDA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5451 | N70G, V98E, | MRSSPGNMERIVICLMVIFLGTLVHKSSSQGQDRHMIRMRQ | 0.704 | 1016 |
| | P107V, R115D | LIDIVDQLKNYVNDLVPEFLPAPEDVETGCEWSAFSCFQKA | | |
| | | QLKSANTGNNERIINESIKKLKRKVPSTNAGRDQKHRLTCP | | |
| | | SCDSYEKKPPKEFLERFKSLLQKMIHQHLSSRTHGSEDS | | |
| IL-21_5693 | K81D, P107V, | MRSSPGNMERIVICLMVIFLGTLVHKSSSQGQDRHMIRMRQ | 0.705 | 1017 |
| | R115D | LIDIVDQLKNYVNDLVPEFLPAPEDVETNCEWSAFSCFQDA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5498 | N70G, K81D, | MRSSPGNMERIVICLMVIFLGTLVHKSSSQGQDRHMIRMRQ | 0.707 | 1018 |
| | P107V, T110D | LIDIVDQLKNYVNDLVPEFLPAPEDVETGCEWSAFSCFQDA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_6162 | H35I, K81D, | MRSSPGNMERIVICLMVIFLGTLVHKSSSQGQDRIMIRMRQ | 0.708 | 1019 |
| | V98E, R114G | LIDIVDQLKNYVNDLVPEFLPAPEDVETNCEWSAFSCFQDA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5301 | K81D, V98E, | MRSSPGNMERIVICLMVIFLGTLVHKSSSQGQDRHMIRMRQ | 0.71 | 1020 |
| | P107V, R119T | LIDIVDQLKNYVNDLVPEFLPAPEDVETNCEWSAFSCFQDA | | |
| | | QLKSANTGNNERIINESIKKLKRKVPSTNAGRRQKHTLTCP | | |
| | | SCDSYEKKPPKEFLERFKSLLQKMIHQHLSSRTHGSEDS | | |
| IL-21_5211 | K81D, T110D, | MRSSPGNMERIVICLMVIFLGTLVHKSSSQGQDRHMIRMRQ | 0.711 | 1021 |
| | R115G, H118D | LIDIVDQLKNYVNDLVPEFLPAPEDVETNCEWSAFSCFQDA | | |
| | | QLKSANTGNNERIINVSIKKLKRKPPSDNAGRGQKDRLTCP | | |
| | | SCDSYEKKPPKEFLERFKSLLQKMIHQHLSSRTHGSEDS | | |
| IL-21_5430 | N70G, V98E, | MRSSPGNMERIVICLMVIFLGTLVHKSSSQGQDRHMIRMRQ | 0.711 | 1022 |
| | R114Q, R115G | LIDIVDQLKNYVNDLVPEFLPAPEDVETGCEWSAFSCFQKA | | |
| | | QLKSANTGNNERIINESIKKLKRKPPSTNAGQGQKHRLTCP | | |
| | | SCDSYEKKPPKEFLERFKSLLQKMIHQHLSSRTHGSEDS | | |
| IL-21_5284 | K81D, V98E, | MRSSPGNMERIVICLMVIFLGTLVHKSSSQGQDRHMIRMRQ | 0.712 | 1023 |
| | R114Q, R115D | LIDIVDQLKNYVNDLVPEFLPAPEDVETNCEWSAFSCFQDA | | |
| | | QLKSANTGNNERIINESIKKLKRKPPSTNAGQDQKHRLTCP | | |
| | | SCDSYEKKPPKEFLERFKSLLQKMIHQHLSSRTHGSEDS | | |
| IL-21_5223 | K81D, T110D, | MRSSPGNMERIVICLMVIFLGTLVHKSSSQGQDRHMIRMRQ | 0.713 | 1024 |
| | R114Q, R115G | LIDIVDQLKNYVNDLVPEFLPAPEDVETNCEWSAFSCFQDA | | |
| | | QLKSANTGNNERIINVSIKKLKRKPPSDNAGQGQKHRLTCP | | |
| | | SCDSYEKKPPKEFLERFKSLLQKMIHQHLSSRTHGSEDS | | |
| IL-21_6312 | H35F, K81D, | MRSSPGNMERIVICLMVIFLGTLVHKSSSQGQDREMIRMRQ | 0.713 | 1025 |
| | R115D | LIDIVDQLKNYVNDLVPEFLPAPEDVETNCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5234 | K81D, P107V, | MRSSPGNMERIVICLMVIFLGTLVHKSSSQGQDRHMIRMRQ | 0.713 | 1026 |
| | H118D, R119T | LIDIVDQLKNYVNDLVPEFLPAPEDVETNCEWSAFSCFQDA | | |
| | | QLKSANTGNNERIINVSIKKLKRKVPSTNAGRRQKDTLTCP | | |
| | | SCDSYEKKPPKEFLERFKSLLQKMIHQHLSSRTHGSEDS | | |
| IL-21_5467 | N70G, K81D, | MRSSPGNMERIVICLMVIFLGTLVHKSSSQGQDRHMIRMRQ | 0.714 | 1027 |
| | R114G, H118D | LIDIVDQLKNYVNDLVPEFLPAPEDVETGCEWSAFSCFQDA | | |
| | | QLKSANTGNNERIINVSIKKLKRKPPSTNAGGROKDRLTCP | | |
| | | SCDSYEKKPPKEFLERFKSLLOKMIHQHLSSRTHGSEDS | | |
| IL-21_6007 | H35F, K81D, | MRSSPGNMERIVICLMVIFLGTLVHKSSSQGQDREMIRMRQ | 0.714 | 1028 |
| | P107V, R115G | LIDIVDQLKNYVNDLVPEFLPAPEDVETNCEWSAFSCFQDA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5081 | V98E, R114Q, | MRSSPGNMERIVICLMVIFLGTLVHKSSSQGQDRHMIRMRQ | 0.716 | 1029 |
| | R115D, R119T | LIDIVDQLKNYVNDLVPEFLPAPEDVETNCEWSAFSCFQKA | | |
| | | QLKSANTGNNERIINESIKKLKRKPPSTNAGQDQKHTLTCP | | |
| | | SCDSYEKKPPKEFLERFKSLLQKMIHQHLSSRTHGSEDS | | |
| IL-21_5213 | K81D, T110D, | MRSSPGNMERIVICLMVIFLGTLVHKSSSQGQDRHMIRMRQ | 0.718 | 1030 |
| | R115D, R119T | LIDIVDQLKNYVNDLVPEFLPAPEDVETNCEWSAFSCFQDA | | |
| | | QLKSANTGNNERIINVSIKKLKRKPPSDNAGRDQKHTLTCP | | |
| | | SCDSYEKKPPKEFLERFKSLLQKMIHQHLSSRTHGSEDS | | |
| IL-21_5219 | K81D, T110D, | MRSSPGNMERIVICLMVIFLGTLVHKSSSQGQDRHMIRMRQ | 0.718 | 1031 |
| | R114G, R115D | LIDIVDQLKNYVNDLVPEFLPAPEDVETNCEWSAFSCFQDA | | |
| | | QLKSANTGNNERIINVSIKKLKRKPPSDNAGGDQKHRLTCP | | |
| | | SCDSYEKKPPKEFLERFKSLLQKMIHQHLSSRTHGSEDS | | |
| IL-21_5108 | V98E, T110D, | MRSSPGNMERIVICLMVIFLGTLVHKSSSQGQDRHMIRMRQ | 0.718 | 1032 |
| | R115D, T121E | LIDIVDQLKNYVNDLVPEFLPAPEDVETNCEWSAFSCFQKA | | |
| | | QLKSANTGNNERIINESIKKLKRKPPSDNAGRDQKHRLECP | | |
| | | SCDSYEKKPPKEFLERFKSLLQKMIHQHLSSRTHGSEDS | | |
| IL-21_5248 | K81D, P107V, | MRSSPGNMERIVICLMVIFLGTLVHKSSSQGQDRHMIRMRQ | 0.718 | 1033 |
| | R114Q, H118D | LIDIVDQLKNYVNDLVPEFLPAPEDVETNCEWSAFSCFQDA | | |
| | | QLKSANTGNNERIINVSIKKLKRKVPSTNAGQRQKDRLTCP | | |
| | | SCDSYEKKPPKEFLERFKSLLOKMIHQHLSSRTHGSEDS | | |
| IL-21_5289 | K81D, V98E, | MRSSPGNMERIVICLMVIFLGTLVHKSSSQGQDRHMIRMRQ | 0.72 | 1034 |
| | A112P, R115D | LIDIVDQLKNYVNDLVPEFLPAPEDVETNCEWSAFSCFQDA | | |
| | | QLKSANTGNNERIINESIKKLKRKPPSTNPGRDQKHRLTCP | | |
| | | SCDSYEKKPPKEFLERFKSLLQKMIHQHLSSRTHGSEDS | | |
| IL-21_5067 | V98E, R114G, | MRSSPGNMERIVICLMVIFLGTLVHKSSSQGQDRHMIRMRQ | 0.72 | 1035 |
| | H118D, R119T | LIDIVDQLKNYVNDLVPEFLPAPEDVETNCEWSAFSCFQKA | | |
| | | QLKSANTGNNERIINESIKKLKRKPPSTNAGGROKDTLTCP | | |
| | | SCDSYEKKPPKEFLERFKSLLQKMIHQHLSSRTHGSEDS | | |
| IL-21_6051 | H35F, N70G, | MRSSPGNMERIVICLMVIFLGTLVHKSSSQGQDRFMIRMRQ | 0.72 | 1036 |
| | K81D, T110D | LIDIVDQLKNYVNDLVPEFLPAPEDVETGCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_6127 | H35I, V98E, | MRSSPGNMERIVICLMVIFLGTLVHKSSSQGQDRIMIRMRQ | 0.72 | 1037 |
| | A112P, R115D | LIDIVDQLKNYVNDLVPEFLPAPEDVETNCEWSAFSCFQKA | | |
| | | QLKSANTGNNERIINESIKKLKRKPPSTNPGRDQKHRLTCP | | |
| | | SCDSYEKKPPKEFLERFKSLLQKMIHQHLSSRTHGSEDS | | |
| IL-21_5425 | N70G, V98E, | MRSSPGNMERIVICLMVIFLGTLVHKSSSQGQDRHMIRMRQ | 0.722 | 1038 |
| | R114G, R115G | LIDIVDQLKNYVNDLVPEFLPAPEDVETGCEWSAFSCFQKA | | |
| | | QLKSANTGNNERIINESIKKLKRKPPSTNAGGGQKHRLTCP | | |
| | | SCDSYEKKPPKEFLERFKSLLOKMIHOHLSSRTHGSEDS | | |
| IL-21_5074 | V98E, R114Q, | MRSSPGNMERIVICLMVIFLGTLVHKSSSQGQDRHMIRMRQ | 0.722 | 1039 |
| | R119T, T121E | LIDIVDQLKNYVNDLVPEFLPAPEDVETNCEWSAFSCFQKA | | |
| | | QLKSANTGNNERIINESIKKLKRKPPSTNAGQRQKHTLECP | | |
| | | SCDSYEKKPPKEFLERFKSLLQKMIHQHLSSRTHGSEDS | | |
| IL-21_5442 | N70G, V98E, | MRSSPGNMERIVICLMVIFLGTLVHKSSSQGQDRHMIRMRQ | 0.722 | 1040 |
| | T110D, R115G | LIDIVDQLKNYVNDLVPEFLPAPEDVETGCEWSAFSCFQKA | | |
| | | QLKSANTGNNERIINESIKKLKRKPPSDNAGRGQKHRLTCP | | |
| | | SCDSYEKKPPKEFLERFKSLLQKMIHQHLSSRTHGSEDS | | |
| IL-21_6144 | H35I, K81D, | MRSSPGNMERIVICLMVIFLGTLVHKSSSQGQDRIMIRMRQ | 0.723 | 1041 |
| | R114Q, R115D | LIDIVDQLKNYVNDLVPEFLPAPEDVETNCEWSAFSCFQDA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5221 | K81D, T110D, | MRSSPGNMERIVICLMVIFLGTLVHKSSSQGQDRHMIRMRQ | 0.723 | 1042 |
| | R114Q, R119T | LIDIVDQLKNYVNDLVPEFLPAPEDVETNCEWSAFSCFQDA | | |
| | | QLKSANTGNNERIINVSIKKLKRKPPSDNAGORQKHTLTCP | | |
| | | SCDSYEKKPPKEFLERFKSLLQKMIHQHLSSRTHGSEDS | | |
| IL-21_6010 | H35F, K81D, | MRSSPGNMERIVICLMVIFLGTLVHKSSSQGQDREMIRMRQ | 0.724 | 1043 |
| | P107V, R114Q | LIDIVDQLKNYVNDLVPEFLPAPEDVETNCEWSAFSCFQDA | | |
| | | QLKSANTGNNERIINVSIKKLKRKVPSTNAGORQKHRLTCP | | |
| | | SCDSYEKKPPKEFLERFKSLLQKMIHQHLSSRTHGSEDS | | |
| IL-21_6000 | H35F, K81D, | MRSSPGNMERIVICLMVIFLGTLVHKSSSQGQDRFMIRMRQ | 0.724 | 1044 |
| | A112P, R114G | LIDIVDQLKNYVNDLVPEFLPAPEDVETNCEWSAFSCFQDA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5701 | K81D, V98E, | MRSSPGNMERIVICLMVIFLGTLVHKSSSQGQDRHMIRMRQ | 0.724 | 1045 |
| | R115G | LIDIVDQLKNYVNDLVPEFLPAPEDVETNCEWSAFSCFQDA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_6136 | H35I, V98E, | MRSSPGNMERIVICLMVIFLGTLVHKSSSQGQDRIMIRMRQ | 0.726 | 1046 |
| | P107V, R115D | LIDIVDQLKNYVNDLVPEFLPAPEDVETNCEWSAFSCFQKA | | |
| | | QLKSANTGNNERIINESIKKLKRKVPSTNAGRDQKHRLTCP | | |
| | | SCDSYEKKPPKEFLERFKSLLQKMIHQHLSSRTHGSEDS | | |
| IL-21_5080 | V98E, R114Q, | MRSSPGNMERIVICLMVIFLGTLVHKSSSQGQDRHMIRMRQ | 0.727 | 1047 |
| | R115D, T121E | LIDIVDQLKNYVNDLVPEFLPAPEDVETNCEWSAFSCFQKA | | |
| | | QLKSANTGNNERIINESIKKLKRKPPSTNAGQDQKHRLECP | | |
| | | SCDSYEKKPPKEFLERFKSLLQKMIHQHLSSRTHGSEDS | | |
| IL-21_5204 | K81D, A112P, | MRSSPGNMERIVICLMVIFLGTLVHKSSSQGQDRHMIRMRQ | 0.727 | 1048 |
| | R114Q, R115G | LIDIVDQLKNYVNDLVPEFLPAPEDVETNCEWSAFSCFQDA | | |
| | | QLKSANTGNNERIINVSIKKLKRKPPSTNPGQGQKHRLTCP | | |
| | | SCDSYEKKPPKEFLERFKSLLQKMIHQHLSSRTHGSEDS | | |
| IL-21_5899 | I45T, K81D, | MRSSPGNMERIVICLMVIFLGTLVHKSSSQGQDRHMIRMRQ | 0.729 | 1049 |
| | T110D, R115D | LIDTVDQLKNYVNDLVPEFLPAPEDVETNCEWSAFSCFQDA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_5140 | V98E, P107V, | MRSSPGNMERIVICLMVIFLGTLVHKSSSQGQDRHMIRMRQ | 0.729 | 1050 |
| | R114G, R115G | LIDIVDQLKNYVNDLVPEFLPAPEDVETNCEWSAFSCFQKA | | |
| | | QLKSANTGNNERIINESIKKLKRKVPSTNAGGGQKHRLTCP | | |
| | | SCDSYEKKPPKEFLERFKSLLQKMIHQHLSSRTHGSEDS | | |
| IL-21_6001 | H35F, K81D, | MRSSPGNMERIVICLMVIFLGTLVHKSSSQGQDRFMIRMRQ | 0.729 | 1051 |
| | A112P, R114Q | LIDIVDQLKNYVNDLVPEFLPAPEDVETNCEWSAFSCFQDA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5264 | K81D, P107V, | MRSSPGNMERIVICLMVIFLGTLVHKSSSQGQDRHMIRMRQ | 0.73 | 1052 |
| | T110D, R114Q | LIDIVDQLKNYVNDLVPEFLPAPEDVETNCEWSAFSCFQDA | | |
| | | QLKSANTGNNERIINVSIKKLKRKVPSDNAGQRQKHRLTCP | | |
| | | SCDSYEKKPPKEFLERFKSLLQKMIHQHLSSRTHGSEDS | | |
| IL-21_5755 | N70G, V98E, | MRSSPGNMERIVICLMVIFLGTLVHKSSSQGQDRHMIRMRQ | 0.73 | 1053 |
| | 1R15D | LIDIVDQLKNYVNDLVPEFLPAPEDVETGCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5096 | V98E, A112P, | MRSSPGNMERIVICLMVIFLGTLVHKSSSQGQDRHMIRMRQ | 0.73 | 1054 |
| | R114G, R115D | LIDIVDQLKNYVNDLVPEFLPAPEDVETNCEWSAFSCFQKA | | |
| | | QLKSANTGNNERIINESIKKLKRKPPSTNPGGDQKHRLTCP | | |
| | | SCDSYEKKPPKEFLERFKSLLQKMIHQHLSSRTHGSEDS | | |
| IL-21_6131 | H35I, V98E, | MRSSPGNMERIVICLMVIFLGTLVHKSSSQGQDRIMIRMRQ | 0.73 | 1055 |
| | T110D, R115D | LIDIVDQLKNYVNDLVPEFLPAPEDVETNCEWSAFSCFQKA | | |
| | | QLKSANTGNNERIINESIKKLKRKPPSDNAGRDQKHRLTCP | | |
| | | SCDSYEKKPPKEFLERFKSLLQKMIHQHLSSRTHGSEDS | | |
| IL-21_5294 | K81D, V98E, | MRSSPGNMERIVICLMVIFLGTLVHKSSSQGQDRHMIRMRQ | 0.73 | 1056 |
| | T110D, H118D | LIDIVDQLKNYVNDLVPEFLPAPEDVETNCEWSAFSCFQDA | | |
| | | QLKSANTGNNERIINESIKKLKRKPPSDNAGRRQKDRLTCP | | |
| | | SCDSYEKKPPKEFLERFKSLLQKMIHQHLSSRTHGSEDS | | |
| IL-21_5216 | K81D, T110D, | MRSSPGNMERIVICLMVIFLGTLVHKSSSQGQDRHMIRMRQ | 0.731 | 1057 |
| | R114G, R119T | LIDIVDQLKNYVNDLVPEFLPAPEDVETNCEWSAFSCFQDA | | |
| | | QLKSANTGNNERIINVSIKKLKRKPPSDNAGGRQKHTLTCP | | |
| | | SCDSYEKKPPKEFLERFKSLLQKMIHQHLSSRTHGSEDS | | |
| IL-21_5243 | K81D, P107V, | MRSSPGNMERIVICLMVIFLGTLVHKSSSQGQDRHMIRMRQ | 0.731 | 1058 |
| | R114G, H118 | LIDIVDQLKNYVNDLVPEFLPAPEDVETNCEWSAFSCFQDA | | |
| | D | QLKSANTGNNERIINVSIKKLKRKVPSTNAGGRQKDRLTCP | | |
| | | SCDSYEKKPPKEFLERFKSLLQKMIHQHLSSRTHGSEDS | | |
| IL-21_6130 | H35I, V98E, | MRSSPGNMERIVICLMVIFLGTLVHKSSSQGQDRIMIRMRQ | 0.731 | 1059 |
| | T110D, R115G | LIDIVDQLKNYVNDLVPEFLPAPEDVETNCEWSAFSCFQKA | | |
| | | QLKSANTGNNERIINESIKKLKRKPPSDNAGRGQKHRLTCP | | |
| | | SCDSYEKKPPKEFLERFKSLLQKMIHQHLSSRTHGSEDS | | |
| IL-21_5259 | K81D, P107V, | MRSSPGNMERIVICLMVIFLGTLVHKSSSQGQDRHMIRMRQ | 0.731 | 1060 |
| | T110D, R119T | LIDIVDQLKNYVNDLVPEFLPAPEDVETNCEWSAFSCFQDA | | |
| | | QLKSANTGNNERIINVSIKKLKRKVPSDNAGRRQKHTLTCP | | |
| | | SCDSYEKKPPKEFLERFKSLLQKMIHQHLSSRTHGSEDS | | |
| IL-21_5441 | N70G, V98E, | MRSSPGNMERIVICLMVIFLGTLVHKSSSQGQDRHMIRMRQ | 0.731 | 1061 |
| | T110D, H118D | LIDIVDQLKNYVNDLVPEFLPAPEDVETGCEWSAFSCFQKA | | |
| | | QLKSANTGNNERIINESIKKLKRKPPSDNAGRRQKDRLTCP | | |
| | | SCDSYEKKPPKEFLERFKSLLQKMIHQHLSSRTHGSEDS | | |
| IL-21_5069 | V98E, R114G, | MRSSPGNMERIVICLMVIFLGTLVHKSSSQGQDRHMIRMRQ | 0.731 | 1062 |
| | R115G, R119T | LIDIVDQLKNYVNDLVPEFLPAPEDVETNCEWSAFSCFQKA | | |
| | | QLKSANTGNNERIINESIKKLKRKPPSTNAGGGQKHTLTCP | | |
| | | SCDSYEKKPPKEFLERFKSLLQKMIHQHLSSRTHGSEDS | | |
| IL-21_6048 | H35F, N70G, | MRSSPGNMERIVICLMVIFLGTLVHKSSSQGQDRFMIRMRQ | 0.732 | 1063 |
| | K81D, R114G | LIDIVDQLKNYVNDLVPEFLPAPEDVETGCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5466 | N70G, K81D, | MRSSPGNMERIVICLMVIFLGTLVHKSSSQGQDRHMIRMRQ | 0.733 | 1064 |
| | R114G, R119T | LIDIVDQLKNYVNDLVPEFLPAPEDVETGCEWSAFSCFQDA | | |
| | | QLKSANTGNNERIINVSIKKLKRKPPSTNAGGRQKHTLTCP | | |
| | | SCDSYEKKPPKEFLERFKSLLQKMIHQHLSSRTHGSEDS | | |
| IL-21_5278 | K81D, V98E, | MRSSPGNMERIVICLMVIFLGTLVHKSSSQGQDRHMIRMRQ | 0.733 | 1065 |
| | R114G, R115G | LIDIVDQLKNYVNDLVPEFLPAPEDVETNCEWSAFSCFQDA | | |
| | | QLKSANTGNNERIINESIKKLKRKPPSTNAGGGQKHRLTCP | | |
| | | SCDSYEKKPPKEFLERFKSLLQKMIHQHLSSRTHGSEDS | | |
| IL-21_5275 | K81D, V98E, | MRSSPGNMERIVICLMVIFLGTLVHKSSSQGQDRHMIRMRQ | 0.733 | 1066 |
| | R114G, T121E | LIDIVDQLKNYVNDLVPEFLPAPEDVETNCEWSAFSCFQDA | | |
| | | QLKSANTGNNERIINESIKKLKRKPPSTNAGGRQKHRLECP | | |
| | | SCDSYEKKPPKEFLERFKSLLQKMIHQHLSSRTHGSEDS | | |
| IL-21_5117 | V98E, T110D, | MRSSPGNMERIVICLMVIFLGTLVHKSSSQGQDRHMIRMRQ | 0.733 | 1067 |
| | R114Q, R119T | LIDIVDQLKNYVNDLVPEFLPAPEDVETNCEWSAFSCFQKA | | |
| | | QLKSANTGNNERIINESIKKLKRKPPSDNAGQRQKHTLTCP | | |
| | | SCDSYEKKPPKEFLERFKSLLQKMIHQHLSSRTHGSEDS | | |
| IL-21_6047 | H35F, N70G, | MRSSPGNMERIVICLMVIFLGTLVHKSSSQGQDREMIRMRQ | 0.733 | 1068 |
| | K81D, R115D | LIDIVDQLKNYVNDLVPEFLPAPEDVETGCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5203 | K81D, A112P, | MRSSPGNMERIVICLMVIFLGTLVHKSSSQGQDRHMIRMRQ | 0.734 | 1069 |
| | R114Q, H118D | LIDIVDQLKNYVNDLVPEFLPAPEDVETNCEWSAFSCFQDA | | |
| | | QLKSANTGNNERIINVSIKKLKRKPPSTNPGQRQKDRLTCP | | |
| | | SCDSYEKKPPKEFLERFKSLLQKMIHQHLSSRTHGSEDS | | |
| IL-21_5495 | N70G, K81D, | MRSSPGNMERIVICLMVIFLGTLVHKSSSQGQDRHMIRMRQ | 0.737 | 1070 |
| | 1P07V, R114G | LIDIVDQLKNYVNDLVPEFLPAPEDVETGCEWSAFSCFQDA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5414 | N70G, V98E, | MRSSPGNMERIVICLMVIFLGTLVHKSSSQGQDRHMIRMRQ | 0.737 | 1071 |
| | H118D, T121E | LIDIVDQLKNYVNDLVPEFLPAPEDVETGCEWSAFSCFQKA | | |
| | | QLKSANTGNNERIINESIKKLKRKPPSTNAGRRQKDRLECP | | |
| | | SCDSYEKKPPKEFLERFKSLLQKMIHQHLSSRTHGSEDS | | |
| IL-21_5244 | K81D, P107V, | MRSSPGNMERIVICLMVIFLGTLVHKSSSQGQDRHMIRMRQ | 0.737 | 1072 |
| | R114G, R115G | LIDIVDQLKNYVNDLVPEFLPAPEDVETNCEWSAFSCFQDA | | |
| | | QLKSANTGNNERIINVSIKKLKRKVPSTNAGGGQKHRLTCP | | |
| | | SCDSYEKKPPKEFLERFKSLLQKMIHQHLSSRTHGSEDS | | |
| IL-21_6046 | H35F, N70G, | MRSSPGNMERIVICLMVIFLGTLVHKSSSQGQDRFMIRMRQ | 0.738 | 1073 |
| | K81D, R115G | LIDIVDQLKNYVNDLVPEFLPAPEDVETGCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_6154 | H35I, K81D, | MRSSPGNMERIVICLMVIFLGTLVHKSSSQGQDRIMIRMRQ | 0.738 | 1074 |
| | P107V, R115G | LIDIVDQLKNYVNDLVPEFLPAPEDVETNCEWSAFSCFQDA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_6185 | H35I, N70G, | MRSSPGNMERIVICLMVIFLGTLVHKSSSQGQDRIMIRMRQ | 0.738 | 1075 |
| | V98E, R115G | LIDIVDQLKNYVNDLVPEFLPAPEDVETGCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_6053 | H35F, N70G, | MRSSPGNMERIVICLMVIFLGTLVHKSSSQGQDREMIRMRQ | 0.739 | 1076 |
| | 8K1D, V98E | LIDIVDQLKNYVNDLVPEFLPAPEDVETGCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5077 | V98E, R114Q, | MRSSPGNMERIVICLMVIFLGTLVHKSSSQGQDRHMIRMRQ | 0.739 | 1077 |
| | R115G, T121E | LIDIVDQLKNYVNDLVPEFLPAPEDVETNCEWSAFSCFQKA | | |
| | | QLKSANTGNNERIINESIKKLKRKPPSTNAGQGQKHRLECP | | |
| | | SCDSYEKKPPKEFLERFKSLLQKMIHQHLSSRTHGSEDS | | |
| IL-21_5245 | K81D, P107V, | MRSSPGNMERIVICLMVIFLGTLVHKSSSQGQDRHMIRMRQ | 0.74 | 1078 |
| | R114G, R115D | LIDIVDQLKNYVNDLVPEFLPAPEDVETNCEWSAFSCFQDA | | |
| | | QLKSANTGNNERIINVSIKKLKRKVPSTNAGGDQKHRLTCP | | |
| | | SCDSYEKKPPKEFLERFKSLLQKMIHQHLSSRTHGSEDS | | |
| IL-21_6229 | H35I, I45T, | MRSSPGNMERIVICLMVIFLGTLVHKSSSQGQDRIMIRMRQ | 0.74 | 1079 |
| | K81D, R114Q | LIDTVDQLKNYVNDLVPEFLPAPEDVETNCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5464 | N70G, K81D, | MRSSPGNMERIVICLMVIFLGTLVHKSSSQGQDRHMIRMRQ | 0.74 | 1080 |
| | R115D, H118D | LIDIVDQLKNYVNDLVPEFLPAPEDVETGCEWSAFSCFQDA | | |
| | | QLKSANTGNNERIINVSIKKLKRKPPSTNAGRDQKDRLTCP | | |
| | | SCDSYEKKPPKEFLERFKSLLQKMIHQHLSSRTHGSEDS | | |
| IL-21_5217 | K81D, T110D, | MRSSPGNMERIVICLMVIFLGTLVHKSSSQGQDRHMIRMRQ | 0.74 | 1081 |
| | R114G, H118D | LIDIVDQLKNYVNDLVPEFLPAPEDVETNCEWSAFSCFQDA | | |
| | | QLKSANTGNNERIINVSIKKLKRKPPSDNAGGRQKDRLTCP | | |
| | | SCDSYEKKPPKEFLERFKSLLQKMIHQHLSSRTHGSEDS | | |
| IL-21_5261 | K81D, P107V, | MRSSPGNMERIVICLMVIFLGTLVHKSSSQGQDRHMIRMRQ | 0.74 | 1082 |
| | T110D, R115G | LIDIVDQLKNYVNDLVPEFLPAPEDVETNCEWSAFSCFQDA | | |
| | | QLKSANTGNNERIINVSIKKLKRKVPSDNAGRGQKHRLTCP | | |
| | | SCDSYEKKPPKEFLERFKSLLQKMIHQHLSSRTHGSEDS | | |
| IL-21_5501 | N70G, K81D, | MRSSPGNMERIVICLMVIFLGTLVHKSSSQGQDRHMIRMRQ | 0.74 | 1083 |
| | V98E, H118D | LIDIVDQLKNYVNDLVPEFLPAPEDVETGCEWSAFSCFQDA | | |
| | | QLKSANTGNNERIINESIKKLKRKPPSTNAGRRQKDRLTCP | | |
| | | SCDSYEKKPPKEFLERFKSLLQKMIHQHLSSRTHGSEDS | | |
| IL-21_5179 | K81D, R114Q, | MRSSPGNMERIVICLMVIFLGTLVHKSSSQGQDRHMIRMRQ | 0.741 | 1084 |
| | H118D, T121E | LIDIVDQLKNYVNDLVPEFLPAPEDVETNCEWSAFSCFQDA | | |
| | | QLKSANTGNNERIINVSIKKLKRKPPSTNAGQRQKDRLECP | | |
| | | SCDSYEKKPPKEFLERFKSLLQKMIHQHLSSRTHGSEDS | | |
| IL-21_5436 | N70G, V98E, | MRSSPGNMERIVICLMVIFLGTLVHKSSSQGQDRHMIRMRQ | 0.742 | 1085 |
| | A112P, R115D | LIDIVDQLKNYVNDLVPEFLPAPEDVETGCEWSAFSCFQKA | | |
| | | QLKSANTGNNERIINESIKKLKRKPPSTNPGRDQKHRLTCP | | |
| | | SCDSYEKKPPKEFLERFKSLLQKMIHQHLSSRTHGSEDS | | |
| IL-21_6081 | H35F, I45T, | MRSSPGNMERIVICLMVIFLGTLVHKSSSQGQDREMIRMRQ | 0.742 | 1086 |
| | 1K8D, R115D | LIDTVDQLKNYVNDLVPEFLPAPEDVETNCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5114 | V98E, T110D, | MRSSPGNMERIVICLMVIFLGTLVHKSSSQGQDRHMIRMRQ | 0.742 | 1087 |
| | R114G, R115G | LIDIVDQLKNYVNDLVPEFLPAPEDVETNCEWSAFSCFQKA | | |
| | | QLKSANTGNNERIINESIKKLKRKPPSDNAGGGQKHRLTCP | | |
| | | SCDSYEKKPPKEFLERFKSLLQKMIHQHLSSRTHGSEDS | | |
| IL-21_5078 | V98E, R114Q, | MRSSPGNMERIVICLMVIFLGTLVHKSSSQGQDRHMIRMRQ | 0.743 | 1088 |
| | R115G, R119T | LIDIVDQLKNYVNDLVPEFLPAPEDVETNCEWSAFSCFQKA | | |
| | | QLKSANTGNNERIINESIKKLKRKPPSTNAGQGQKHTLTCP | | |
| | | SCDSYEKKPPKEFLERFKSLLQKMIHQHLSSRTHGSEDS | | |
| IL-21_5949 | I45T, N70G, | MRSSPGNMERIVICLMVIFLGTLVHKSSSQGQDRHMIRMRQ | 0.743 | 1089 |
| | K81D, V98E | LIDTVDQLKNYVNDLVPEFLPAPEDVETGCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5103 | V98E, T110D, | MRSSPGNMERIVICLMVIFLGTLVHKSSSQGQDRHMIRMRQ | 0.744 | 1090 |
| | H118D, T121E | LIDIVDQLKNYVNDLVPEFLPAPEDVETNCEWSAFSCFQKA | | |
| | | QLKSANTGNNERIINESIKKLKRKPPSDNAGRRQKDRLECP | | |
| | | SCDSYEKKPPKEFLERFKSLLQKMIHQHLSSRTHGSEDS | | |
| IL-21_5062 | V98E, R115D, | MRSSPGNMERIVICLMVIFLGTLVHKSSSQGQDRHMIRMRQ | 0.744 | 1091 |
| | R119T, T121E | LIDIVDQLKNYVNDLVPEFLPAPEDVETNCEWSAFSCFQKA | | |
| | | QLKSANTGNNERIINESIKKLKRKPPSTNAGRDQKHTLECP | | |
| | | SCDSYEKKPPKEFLERFKSLLQKMIHQHLSSRTHGSEDS | | |
| IL-21_5984 | H35F, V98E, | MRSSPGNMERIVICLMVIFLGTLVHKSSSQGQDRFMIRMRQ | 0.745 | 1092 |
| | T110D, R115D | LIDIVDQLKNYVNDLVPEFLPAPEDVETNCEWSAFSCFQKA | | |
| | | QLKSANTGNNERIINESIKKLKRKPPSDNAGRDQKHRLTCP | | |
| | | SCDSYEKKPPKEFLERFKSLLQKMIHQHLSSRTHGSEDS | | |
| IL-21_6164 | H35I, K81D, | MRSSPGNMERIVICLMVIFLGTLVHKSSSQGQDRIMIRMRQ | 0.746 | 1093 |
| | V98E, A112P | LIDIVDQLKNYVNDLVPEFLPAPEDVETNCEWSAFSCFQDA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5242 | K81D, P107V, | MRSSPGNMERIVICLMVIFLGTLVHKSSSQGQDRHMIRMRQ | 0.746 | 1094 |
| | R114G, R119T | LIDIVDQLKNYVNDLVPEFLPAPEDVETNCEWSAFSCFQDA | | |
| | | QLKSANTGNNERIINVSIKKLKRKVPSTNAGGRQKHTLTCP | | |
| | | SCDSYEKKPPKEFLERFKSLLQKMIHQHLSSRTHGSEDS | | |
| IL-21_5214 | K81D, T110D, | MRSSPGNMERIVICLMVIFLGTLVHKSSSQGQDRHMIRMRQ | 0.747 | 1095 |
| | R115D, H118D | LIDIVDQLKNYVNDLVPEFLPAPEDVETNCEWSAFSCFQDA | | |
| | | QLKSANTGNNERIINVSIKKLKRKPPSDNAGRDQKDRLTCP | | |
| | | SCDSYEKKPPKEFLERFKSLLQKMIHQHLSSRTHGSEDS | | |
| IL-21_5485 | N70G, K81D, | MRSSPGNMERIVICLMVIFLGTLVHKSSSQGQDRHMIRMRQ | 0.747 | 1096 |
| | T110D, R115G | LIDIVDQLKNYVNDLVPEFLPAPEDVETGCEWSAFSCFQDA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_5186 | K81D, R114Q, | MRSSPGNMERIVICLMVIFLGTLVHKSSSQGQDRHMIRMRQ | 0.748 | 1097 |
| | R115D, H118D | LIDIVDQLKNYVNDLVPEFLPAPEDVETNCEWSAFSCFQDA | | |
| | | QLKSANTGNNERIINVSIKKLKRKPPSTNAGQDQKDRLTCP | | |
| | | SCDSYEKKPPKEFLERFKSLLQKMIHQHLSSRTHGSEDS | | |
| IL-21_6009 | H35F, K81D, | MRSSPGNMERIVICLMVIFLGTLVHKSSSQGQDREMIRMRQ | 0.749 | 1098 |
| | 1P07V, R114G | LIDIVDQLKNYVNDLVPEFLPAPEDVETNCEWSAFSCFQDA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5494 | N70G, K81D, | MRSSPGNMERIVICLMVIFLGTLVHKSSSQGQDRHMIRMRQ | 0.75 | 1099 |
| | P107V, R115D | LIDIVDQLKNYVNDLVPEFLPAPEDVETGCEWSAFSCFQDA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5424 | N70G, V98E, | MRSSPGNMERIVICLMVIFLGTLVHKSSSQGQDRHMIRMRQ | 0.75 | 1100 |
| | R114G, H118D | LIDIVDQLKNYVNDLVPEFLPAPEDVETGCEWSAFSCFQKA | | |
| | | QLKSANTGNNERIINESIKKLKRKPPSTNAGGRQKDRLTCP | | |
| | | SCDSYEKKPPKEFLERFKSLLQKMIHQHLSSRTHGSEDS | | |
| IL-21_5499 | N70G, K81D, | MRSSPGNMERIVICLMVIFLGTLVHKSSSQGQDRHMIRMRQ | 0.751 | 1101 |
| | V98E, T121E | LIDIVDQLKNYVNDLVPEFLPAPEDVETGCEWSAFSCFQDA | | |
| | | QLKSANTGNNERIINESIKKLKRKPPSTNAGRRQKHRLECP | | |
| | | SCDSYEKKPPKEFLERFKSLLQKMIHQHLSSRTHGSEDS | | |
| IL-21_5156 | V98E, P107V, | MRSSPGNMERIVICLMVIFLGTLVHKSSSQGQDRHMIRMRQ | 0.752 | 1102 |
| | T110D, H118D | LIDIVDQLKNYVNDLVPEFLPAPEDVETNCEWSAFSCFQKA | | |
| | | QLKSANTGNNERIINESIKKLKRKVPSDNAGRRQKDRLTCP | | |
| | | SCDSYEKKPPKEFLERFKSLLQKMIHQHLSSRTHGSEDS | | |
| IL-21_5109 | V98E, T110D, | MRSSPGNMERIVICLMVIFLGTLVHKSSSQGQDRHMIRMRQ | 0.752 | 1103 |
| | R115D, R119T | LIDIVDQLKNYVNDLVPEFLPAPEDVETNCEWSAFSCFQKA | | |
| | | QLKSANTGNNERIINESIKKLKRKPPSDNAGRDQKHTLTCP | | |
| | | SCDSYEKKPPKEFLERFKSLLQKMIHQHLSSRTHGSEDS | | |
| IL-21_6220 | H351, I45T, | MRSSPGNMERIVICLMVIFLGTLVHKSSSQGQDRIMIRMRQ | 0.753 | 1104 |
| | V98E, R115D | LIDTVDQLKNYVNDLVPEFLPAPEDVETNCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5184 | K81D, R114Q, | MRSSPGNMERIVICLMVIFLGTLVHKSSSQGQDRHMIRMRQ | 0.753 | 1105 |
| | R115D, T121E | LIDIVDQLKNYVNDLVPEFLPAPEDVETNCEWSAFSCFQDA | | |
| | | QLKSANTGNNERIINVSIKKLKRKPPSTNAGQDQKHRLECP | | |
| | | SCDSYEKKPPKEFLERFKSLLQKMIHQHLSSRTHGSEDS | | |
| IL-21_5420 | N70G, V98E, | MRSSPGNMERIVICLMVIFLGTLVHKSSSQGQDRHMIRMRQ | 0.755 | 1106 |
| | R115D, R119T | LIDIVDQLKNYVNDLVPEFLPAPEDVETGCEWSAFSCFQKA | | |
| | | QLKSANTGNNERIINESIKKLKRKPPSTNAGRDQKHTLTCP | | |
| | | SCDSYEKKPPKEFLERFKSLLQKMIHQHLSSRTHGSEDS | | |
| IL-21_5673 | K81D, R114Q, | MRSSPGNMERIVICLMVIFLGTLVHKSSSQGQDRHMIRMRQ | 0.755 | 1107 |
| | R115D | LIDIVDQLKNYVNDLVPEFLPAPEDVETNCEWSAFSCFQDA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5255 | K81D, P107V, | MRSSPGNMERIVICLMVIFLGTLVHKSSSQGQDRHMIRMRQ | 0.756 | 1108 |
| | A112P, R115D | LIDIVDQLKNYVNDLVPEFLPAPEDVETNCEWSAFSCFQDA | | |
| | | QLKSANTGNNERIINVSIKKLKRKVPSTNPGRDQKHRLTCP | | |
| | | SCDSYEKKPPKEFLERFKSLLQKMIHQHLSSRTHGSEDS | | |
| IL-21_5506 | N70G, K81D, | MRSSPGNMERIVICLMVIFLGTLVHKSSSQGQDRHMIRMRQ | 0.756 | 1109 |
| | V98E, A112P | LIDIVDQLKNYVNDLVPEFLPAPEDVETGCEWSAFSCFQDA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5195 | K81D, A112P, | MRSSPGNMERIVICLMVIFLGTLVHKSSSQGQDRHMIRMRQ | 0.757 | 1110 |
| | R115D, H118D | LIDIVDQLKNYVNDLVPEFLPAPEDVETNCEWSAFSCFQDA | | |
| | | QLKSANTGNNERIINVSIKKLKRKPPSTNPGRDQKDRLTCP | | |
| | | SCDSYEKKPPKEFLERFKSLLQKMIHQHLSSRTHGSEDS | | |
| IL-21_5361 | N70G, T110D, | MRSSPGNMERIVICLMVIFLGTLVHKSSSQGQDRHMIRMRQ | 0.757 | 1111 |
| | R115D, H118D | LIDIVDQLKNYVNDLVPEFLPAPEDVETGCEWSAFSCFQKA | | |
| | | QLKSANTGNNERIINVSIKKLKRKPPSDNAGRDQKDRLTCP | | |
| | | SCDSYEKKPPKEFLERFKSLLQKMIHQHLSSRTHGSEDS | | |
| IL-21_5908 | I45T, K81D, | MRSSPGNMERIVICLMVIFLGTLVHKSSSQGQDRHMIRMRQ | 0.758 | 1112 |
| | P107V, T110D | LIDTVDQLKNYVNDLVPEFLPAPEDVETNCEWSAFSCFQDA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5123 | V98E, T110D, | MRSSPGNMERIVICLMVIFLGTLVHKSSSQGQDRHMIRMRQ | 0.759 | 1113 |
| | A112P, H118D | LIDIVDQLKNYVNDLVPEFLPAPEDVETNCEWSAFSCFQKA | | |
| | | QLKSANTGNNERIINESIKKLKRKPPSDNPGRRQKDRLTCP | | |
| | | SCDSYEKKPPKEFLERFKSLLQKMIHQHLSSRTHGSEDS | | |
| IL-21_5240 | K81D, P107V, | MRSSPGNMERIVICLMVIFLGTLVHKSSSQGQDRHMIRMRQ | 0.759 | 1114 |
| | R115D, H118D | LIDIVDQLKNYVNDLVPEFLPAPEDVETNCEWSAFSCFQDA | | |
| | | QLKSANTGNNERIINVSIKKLKRKVPSTNAGRDQKDRLTCP | | |
| | | SCDSYEKKPPKEFLERFKSLLQKMIHQHLSSRTHGSEDS | | |
| IL-21_5163 | K81D, R115G, | MRSSPGNMERIVICLMVIFLGTLVHKSSSQGQDRHMIRMRQ | 0.761 | 1115 |
| | R119T, T121E | LIDIVDQLKNYVNDLVPEFLPAPEDVETNCEWSAFSCFQDA | | |
| | | QLKSANTGNNERIINVSIKKLKRKPPSTNAGRGQKHTLECP | | |
| | | SCDSYEKKPPKEFLERFKSLLQKMIHQHLSSRTHGSEDS | | |
| IL-21_5667 | K81D, R114G, | MRSSPGNMERIVICLMVIFLGTLVHKSSSQGQDRHMIRMRQ | 0.761 | 1116 |
| | R115G | LIDIVDQLKNYVNDLVPEFLPAPEDVETNCEWSAFSCFQDA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5209 | K81D, T110D, | MRSSPGNMERIVICLMVIFLGTLVHKSSSQGQDRHMIRMRQ | 0.762 | 1117 |
| | R115G, T121E | LIDIVDQLKNYVNDLVPEFLPAPEDVETNCEWSAFSCFQDA | | |
| | | QLKSANTGNNERIINVSIKKLKRKPPSDNAGRGQKHRLECP | | |
| | | SCDSYEKKPPKEFLERFKSLLQKMIHQHLSSRTHGSEDS | | |
| IL-21_6311 | H35F, K81D, | MRSSPGNMERIVICLMVIFLGTLVHKSSSQGQDRFMIRMRQ | 0.763 | 1118 |
| | R115G | LIDIVDQLKNYVNDLVPEFLPAPEDVETNCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5105 | V98E, T110D, | MRSSPGNMERIVICLMVIFLGTLVHKSSSQGQDRHMIRMRQ | 0.764 | 1119 |
| | R115G, T121E | LIDIVDQLKNYVNDLVPEFLPAPEDVETNCEWSAFSCFQKA | | |
| | | QLKSANTGNNERIINESIKKLKRKPPSDNAGRGQKHRLECP | | |
| | | SCDSYEKKPPKEFLERFKSLLQKMIHQHLSSRTHGSEDS | | |
| IL-21_5979 | H35F, V98E, | MRSSPGNMERIVICLMVIFLGTLVHKSSSQGQDREMIRMRQ | 0.764 | 1120 |
| | A112P, R115G | LIDIVDQLKNYVNDLVPEFLPAPEDVETNCEWSAFSCFQKA | | |
| | | QLKSANTGNNERIINESIKKLKRKPPSTNPGRGQKHRLTCP | | |
| | | SCDSYEKKPPKEFLERFKSLLQKMIHQHLSSRTHGSEDS | | |
| IL-21_5129 | V98E, P107V, | MRSSPGNMERIVICLMVIFLGTLVHKSSSQGQDRHMIRMRQ | 0.765 | 1121 |
| | H118D, T121E | LIDIVDQLKNYVNDLVPEFLPAPEDVETNCEWSAFSCFQKA | | |
| | | QLKSANTGNNERIINESIKKLKRKVPSTNAGRRQKDRLECP | | |
| | | SCDSYEKKPPKEFLERFKSLLQKMIHQHLSSRTHGSEDS | | |
| IL-21_5180 | K81D, R114Q, | MRSSPGNMERIVICLMVIFLGTLVHKSSSQGQDRHMIRMRQ | 0.765 | 1122 |
| | H118D, R119T | LIDIVDQLKNYVNDLVPEFLPAPEDVETNCEWSAFSCFQDA | | |
| | | QLKSANTGNNERIINVSIKKLKRKPPSTNAGQRQKDTLTCP | | |
| | | SCDSYEKKPPKEFLERFKSLLQKMIHQHLSSRTHGSEDS | | |
| IL-21_5157 | V98E, P107V, | MRSSPGNMERIVICLMVIFLGTLVHKSSSQGQDRHMIRMRQ | 0.765 | 1123 |
| | T110D, R115G | LIDIVDQLKNYVNDLVPEFLPAPEDVETNCEWSAFSCFQKA | | |
| | | QLKSANTGNNERIINESIKKLKRKVPSDNAGRGQKHRLTCP | | |
| | | SCDSYEKKPPKEFLERFKSLLQKMIHQHLSSRTHGSEDS | | |
| IL-21_6126 | H35I, V98E, | MRSSPGNMERIVICLMVIFLGTLVHKSSSQGQDRIMIRMRQ | 0.766 | 1124 |
| | A112P, R115G | LIDIVDQLKNYVNDLVPEFLPAPEDVETNCEWSAFSCFQKA | | |
| | | QLKSANTGNNERIINESIKKLKRKPPSTNPGRGQKHRLTCP | | |
| | | SCDSYEKKPPKEFLERFKSLLQKMIHQHLSSRTHGSEDS | | |
| IL-21_5070 | V98E, R114G, | MRSSPGNMERIVICLMVIFLGTLVHKSSSQGQDRHMIRMRQ | 0.766 | 1125 |
| | R115G, H118D | LIDIVDQLKNYVNDLVPEFLPAPEDVETNCEWSAFSCFQKA | | |
| | | QLKSANTGNNERIINESIKKLKRKPPSTNAGGGQKDRLTCP | | |
| | | SCDSYEKKPPKEFLERFKSLLQKMIHQHLSSRTHGSEDS | | |
| IL-21_5623 | V98E, R114G, | MRSSPGNMERIVICLMVIFLGTLVHKSSSQGQDRHMIRMRQ | 0.767 | 1126 |
| | H118D | LIDIVDQLKNYVNDLVPEFLPAPEDVETNCEWSAFSCFQKA | | |
| | | QLKSANTGNNERIINESIKKLKRKPPSTNAGGRQKDRLTCP | | |
| | | SCDSYEKKPPKEFLERFKSLLQKMIHQHLSSRTHGSEDS | | |
| IL-21_5066 | V98E, R114G, | MRSSPGNMERIVICLMVIFLGTLVHKSSSQGQDRHMIRMRQ | 0.767 | 1127 |
| | H118D, T121E | LIDIVDQLKNYVNDLVPEFLPAPEDVETNCEWSAFSCFQKA | | |
| | | QLKSANTGNNERIINESIKKLKRKPPSTNAGGRQKDRLECP | | |
| | | SCDSYEKKPPKEFLERFKSLLQKMIHQHLSSRTHGSEDS | | |
| IL-21_5229 | K81D, T110D, | MRSSPGNMERIVICLMVIFLGTLVHKSSSQGQDRHMIRMRQ | 0.768 | 1128 |
| | A112P, R115D | LIDIVDQLKNYVNDLVPEFLPAPEDVETNCEWSAFSCFQDA | | |
| | | QLKSANTGNNERIINVSIKKLKRKPPSDNPGRDQKHRLTCP | | |
| | | SCDSYEKKPPKEFLERFKSLLQKMIHQHLSSRTHGSEDS | | |
| IL-21_6199 | H35I, N70G, | MRSSPGNMERIVICLMVIFLGTLVHKSSSQGQDRIMIRMRQ | 0.768 | 1129 |
| | K81D, V98E | LIDIVDQLKNYVNDLVPEFLPAPEDVETGCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_6074 | H35F, I45T, | MRSSPGNMERIVICLMVIFLGTLVHKSSSQGQDRFMIRMRQ | 0.768 | 1130 |
| | V98E, R115D | LIDTVDQLKNYVNDLVPEFLPAPEDVETNCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5419 | N70G, V98E, | MRSSPGNMERIVICLMVIFLGTLVHKSSSQGQDRHMIRMRQ | 0.769 | 1131 |
| | R115D, T121E | LIDIVDQLKNYVNDLVPEFLPAPEDVETGCEWSAFSCFQKA | | |
| | | QLKSANTGNNERIINESIKKLKRKPPSTNAGRDQKHRLECP | | |
| | | SCDSYEKKPPKEFLERFKSLLQKMIHQHLSSRTHGSEDS | | |
| IL-21_5507 | N70G, K81D, | MRSSPGNMERIVICLMVIFLGTLVHKSSSQGQDRHMIRMRQ | 0.77 | 1132 |
| | V98E, T110D | LIDIVDQLKNYVNDLVPEFLPAPEDVETGCEWSAFSCFQDA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_6187 | H35I, N70G, | MRSSPGNMERIVICLMVIFLGTLVHKSSSQGQDRIMIRMRQ | 0.772 | 1133 |
| | V98E, R114G | LIDIVDQLKNYVNDLVPEFLPAPEDVETGCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5910 | I45T, K81D, | MRSSPGNMERIVICLMVIFLGTLVHKSSSQGQDRHMIRMRQ | 0.773 | 1134 |
| | V98E, R115D | LIDTVDQLKNYVNDLVPEFLPAPEDVETNCEWSAFSCFQDA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5088 | V98E, A112P, | MRSSPGNMERIVICLMVIFLGTLVHKSSSQGQDRHMIRMRQ | 0.774 | 1135 |
| | R115G, H118D | LIDIVDQLKNYVNDLVPEFLPAPEDVETNCEWSAFSCFQKA | | |
| | | QLKSANTGNNERIINESIKKLKRKPPSTNPGRGQKDRLTCP | | |
| | | SCDSYEKKPPKEFLERFKSLLQKMIHQHLSSRTHGSEDS | | |
| IL-21_5139 | V98E, P107V, | MRSSPGNMERIVICLMVIFLGTLVHKSSSQGQDRHMIRMRQ | 0.774 | 1136 |
| | R114G, H118D | LIDIVDQLKNYVNDLVPEFLPAPEDVETNCEWSAFSCFQKA | | |
| | | QLKSANTGNNERIINESIKKLKRKVPSTNAGGRQKDRLTCP | | |
| | | SCDSYEKKPPKEFLERFKSLLQKMIHQHLSSRTHGSEDS | | |
| IL-21_5616 | V98E, R115G, | MRSSPGNMERIVICLMVIFLGTLVHKSSSQGQDRHMIRMRQ | 0.775 | 1137 |
| | R119T | LIDIVDQLKNYVNDLVPEFLPAPEDVETNCEWSAFSCFQKA | | |
| | | QLKSANTGNNERIINESIKKLKRKPPSTNAGRGQKHTLTCP | | |
| | | SCDSYEKKPPKEFLERFKSLLQKMIHQHLSSRTHGSEDS | | |
| IL-21_5687 | K81D, T110D, | MRSSPGNMERIVICLMVIFLGTLVHKSSSQGQDRHMIRMRQ | 0.777 | 1138 |
| | R114Q | LIDIVDQLKNYVNDLVPEFLPAPEDVETNCEWSAFSCFQDA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5463 | N70G, K81D, | MRSSPGNMERIVICLMVIFLGTLVHKSSSQGQDRHMIRMRQ | 0.777 | 1139 |
| | R115D, R119T | LIDIVDQLKNYVNDLVPEFLPAPEDVETGCEWSAFSCFQDA | | |
| | | QLKSANTGNNERIINVSIKKLKRKPPSTNAGRDQKHTLTCP | | |
| | | SCDSYEKKPPKEFLERFKSLLQKMIHQHLSSRTHGSEDS | | |
| IL-21_5483 | N70G, K81D, | MRSSPGNMERIVICLMVIFLGTLVHKSSSQGQDRHMIRMRQ | 0.777 | 1140 |
| | T110D, R119T | LIDIVDQLKNYVNDLVPEFLPAPEDVETGCEWSAFSCFQDA | | |
| | | QLKSANTGNNERIINVSIKKLKRKPPSDNAGRRQKHTLTCP | | |
| | | SCDSYEKKPPKEFLERFKSLLQKMIHQHLSSRTHGSEDS | | |
| IL-21_5684 | K81D, T110D, | MRSSPGNMERIVICLMVIFLGTLVHKSSSQGQDRHMIRMRQ | 0.777 | 1141 |
| | R115G | LIDIVDQLKNYVNDLVPEFLPAPEDVETNCEWSAFSCFQDA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_6133 | H35I, V98E, | MRSSPGNMERIVICLMVIFLGTLVHKSSSQGQDRIMIRMRQ | 0.778 | 1142 |
| | T110D, R114Q | LIDIVDQLKNYVNDLVPEFLPAPEDVETNCEWSAFSCFQKA | | |
| | | QLKSANTGNNERIINESIKKLKRKPPSDNAGQRQKHRLTCP | | |
| | | SCDSYEKKPPKEFLERFKSLLQKMIHQHLSSRTHGSEDS | | |
| IL-21_6155 | H35I, K81D, | MRSSPGNMERIVICLMVIFLGTLVHKSSSQGQDRIMIRMRQ | 0.778 | 1143 |
| | P107V, R115D | LIDIVDQLKNYVNDLVPEFLPAPEDVETNCEWSAFSCFQDA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5076 | V98E, R114Q, | MRSSPGNMERIVICLMVIFLGTLVHKSSSQGQDRHMIRMRQ | 0.778 | 1144 |
| | H118D, R119T | LIDIVDQLKNYVNDLVPEFLPAPEDVETNCEWSAFSCFQKA | | |
| | | QLKSANTGNNERIINESIKKLKRKPPSTNAGQRQKDTLTCP | | |
| | | SCDSYEKKPPKEFLERFKSLLQKMIHQHLSSRTHGSEDS | | |
| IL-21_5686 | K81D, T110D, | MRSSPGNMERIVICLMVIFLGTLVHKSSSQGQDRHMIRMRQ | 0.779 | 1145 |
| | R114G | LIDIVDQLKNYVNDLVPEFLPAPEDVETNCEWSAFSCFQDA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5208 | K81D, T110D, | MRSSPGNMERIVICLMVIFLGTLVHKSSSQGQDRHMIRMRQ | 0.779 | 1146 |
| | H118D, R119T | LIDIVDQLKNYVNDLVPEFLPAPEDVETNCEWSAFSCFQDA | | |
| | | QLKSANTGNNERIINVSIKKLKRKPPSDNAGRRQKDTLTCP | | |
| | | SCDSYEKKPPKEFLERFKSLLQKMIHQHLSSRTHGSEDS | | |
| IL-21_5175 | K81D, R114G, | MRSSPGNMERIVICLMVIFLGTLVHKSSSQGQDRHMIRMRQ | 0.78 | 1147 |
| | R115D, T121E | LIDIVDQLKNYVNDLVPEFLPAPEDVETNCEWSAFSCFQDA | | |
| | | QLKSANTGNNERIINVSIKKLKRKPPSTNAGGDQKHRLECP | | |
| | | SCDSYEKKPPKEFLERFKSLLQKMIHQHLSSRTHGSEDS | | |
| IL-21_5418 | N70G, V98E, | MRSSPGNMERIVICLMVIFLGTLVHKSSSQGQDRHMIRMRQ | 0.78 | 1148 |
| | R115G, H118D | LIDIVDQLKNYVNDLVPEFLPAPEDVETGCEWSAFSCFQKA | | |
| | | QLKSANTGNNERIINESIKKLKRKPPSTNAGRGQKDRLTCP | | |
| | | SCDSYEKKPPKEFLERFKSLLQKMIHQHLSSRTHGSEDS | | |
| IL-21_5185 | K81D, R114Q, | MRSSPGNMERIVICLMVIFLGTLVHKSSSQGQDRHMIRMRQ | 0.781 | 1149 |
| | R115D, R119T | LIDIVDQLKNYVNDLVPEFLPAPEDVETNCEWSAFSCFQDA | | |
| | | QLKSANTGNNERIINVSIKKLKRKPPSTNAGQDQKHTLTCP | | |
| | | SCDSYEKKPPKEFLERFKSLLQKMIHQHLSSRTHGSEDS | | |
| IL-21_5155 | V98E, P107V, | MRSSPGNMERIVICLMVIFLGTLVHKSSSQGQDRHMIRMRQ | 0.781 | 1150 |
| | T110D, R119T | LIDIVDQLKNYVNDLVPEFLPAPEDVETNCEWSAFSCFQKA | | |
| | | QLKSANTGNNERIINESIKKLKRKVPSDNAGRRQKHTLTCP | | |
| | | SCDSYEKKPPKEFLERFKSLLQKMIHQHLSSRTHGSEDS | | |
| IL-21_5060 | V98E, R115G, | MRSSPGNMERIVICLMVIFLGTLVHKSSSQGQDRHMIRMRQ | 0.781 | 1151 |
| | H118D, T121E | LIDIVDQLKNYVNDLVPEFLPAPEDVETNCEWSAFSCFQKA | | |
| | | QLKSANTGNNERIINESIKKLKRKPPSTNAGRGQKDRLECP | | |
| | | SCDSYEKKPPKEFLERFKSLLQKMIHQHLSSRTHGSEDS | | |
| IL-21_5171 | K81D, R114G, | MRSSPGNMERIVICLMVIFLGTLVHKSSSQGQDRHMIRMRQ | 0.782 | 1152 |
| | H118D, R119T | LIDIVDQLKNYVNDLVPEFLPAPEDVETNCEWSAFSCFQDA | | |
| | | QLKSANTGNNERIINVSIKKLKRKPPSTNAGGRQKDTLTCP | | |
| | | SCDSYEKKPPKEFLERFKSLLQKMIHQHLSSRTHGSEDS | | |
| IL-21_5132 | V98E, P107V, | MRSSPGNMERIVICLMVIFLGTLVHKSSSQGQDRHMIRMRQ | 0.783 | 1153 |
| | R115G, R119T | LIDIVDQLKNYVNDLVPEFLPAPEDVETNCEWSAFSCFQKA | | |
| | | QLKSANTGNNERIINESIKKLKRKVPSTNAGRGQKHTLTCP | | |
| | | SCDSYEKKPPKEFLERFKSLLQKMIHQHLSSRTHGSEDS | | |
| IL-21_5134 | V98E, P107V, | MRSSPGNMERIVICLMVIFLGTLVHKSSSQGQDRHMIRMRQ | 0.783 | 1154 |
| | R115D, T121E | LIDIVDQLKNYVNDLVPEFLPAPEDVETNCEWSAFSCFQKA | | |
| | | QLKSANTGNNERIINESIKKLKRKVPSTNAGRDQKHRLECP | | |
| | | SCDSYEKKPPKEFLERFKSLLQKMIHQHLSSRTHGSEDS | | |
| IL-21_5983 | H35F, V98E, | MRSSPGNMERIVICLMVIFLGTLVHKSSSQGQDREMIRMRQ | 0.784 | 1155 |
| | T110D, R115G | LIDIVDQLKNYVNDLVPEFLPAPEDVETNCEWSAFSCFQKA | | |
| | | QLKSANTGNNERIINESIKKLKRKPPSDNAGRGQKHRLTCP | | |
| | | SCDSYEKKPPKEFLERFKSLLQKMIHQHLSSRTHGSEDS | | |
| IL-21_5906 | I45T, K81D, | MRSSPGNMERIVICLMVIFLGTLVHKSSSQGQDRHMIRMRQ | 0.784 | 1156 |
| | P107V, R114Q | LIDTVDQLKNYVNDLVPEFLPAPEDVETNCEWSAFSCFQDA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5911 | I45T, K81D, | MRSSPGNMERIVICLMVIFLGTLVHKSSSQGQDRHMIRMRQ | 0.786 | 1157 |
| | V98E, R114G | LIDTVDQLKNYVNDLVPEFLPAPEDVETNCEWSAFSCFQDA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5262 | K81D, P107V, | MRSSPGNMERIVICLMVIFLGTLVHKSSSQGQDRHMIRMRQ | 0.788 | 1158 |
| | T110D, R115D | LIDIVDQLKNYVNDLVPEFLPAPEDVETNCEWSAFSCFQDA | | |
| | | QLKSANTGNNERIINVSIKKLKRKVPSDNAGRDQKHRLTCP | | |
| | | SCDSYEKKPPKEFLERFKSLLQKMIHQHLSSRTHGSEDS | | |
| IL-21_5998 | H35F, K81D, | MRSSPGNMERIVICLMVIFLGTLVHKSSSQGQDRFMIRMRQ | 0.788 | 1159 |
| | A112P, R115G | LIDIVDQLKNYVNDLVPEFLPAPEDVETNCEWSAFSCFQDA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5505 | N70G, K81D, | MRSSPGNMERIVICLMVIFLGTLVHKSSSQGQDRHMIRMRQ | 0.789 | 1160 |
| | V98E, R114Q | LIDIVDQLKNYVNDLVPEFLPAPEDVETGCEWSAFSCFQDA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_5699 | K81D, V98E, | MRSSPGNMERIVICLMVIFLGTLVHKSSSQGQDRHMIRMRQ | 0.789 | 1161 |
| | R119T | LIDIVDQLKNYVNDLVPEFLPAPEDVETNCEWSAFSCFQDA | | |
| | | QLKSANTGNNERIINESIKKLKRKPPSTNAGRRQKHTLTCP | | |
| | | SCDSYEKKPPKEFLERFKSLLQKMIHQHLSSRTHGSEDS | | |
| IL-21_5618 | V98E, R115D, | MRSSPGNMERIVICLMVIFLGTLVHKSSSQGQDRHMIRMRQ | 0.79 | 1162 |
| | T121E | LIDIVDQLKNYVNDLVPEFLPAPEDVETNCEWSAFSCFQKA | | |
| | | QLKSANTGNNERIINESIKKLKRKPPSTNAGRDQKHRLECP | | |
| | | SCDSYEKKPPKEFLERFKSLLQKMIHQHLSSRTHGSEDS | | |
| IL-21_5258 | K81D, P107V, | MRSSPGNMERIVICLMVIFLGTLVHKSSSQGQDRHMIRMRQ | 0.791 | 1163 |
| | T110D, T121E | LIDIVDQLKNYVNDLVPEFLPAPEDVETNCEWSAFSCFQDA | | |
| | | QLKSANTGNNERIINVSIKKLKRKVPSDNAGRRQKHRLECP | | |
| | | SCDSYEKKPPKEFLERFKSLLQKMIHQHLSSRTHGSEDS | | |
| IL-21_5106 | V98E, T110D, | MRSSPGNMERIVICLMVIFLGTLVHKSSSQGQDRHMIRMRQ | 0.792 | 1164 |
| | R115G, R119T | LIDIVDQLKNYVNDLVPEFLPAPEDVETNCEWSAFSCFQKA | | |
| | | QLKSANTGNNERIINESIKKLKRKPPSDNAGRGQKHTLTCP | | |
| | | SCDSYEKKPPKEFLERFKSLLQKMIHQHLSSRTHGSEDS | | |
| IL-21_5269 | K81D, V98E, | MRSSPGNMERIVICLMVIFLGTLVHKSSSQGQDRHMIRMRQ | 0.793 | 1165 |
| | R115G, T121E | LIDIVDQLKNYVNDLVPEFLPAPEDVETNCEWSAFSCFQDA | | |
| | | QLKSANTGNNERIINESIKKLKRKPPSTNAGRGQKHRLECP | | |
| | | SCDSYEKKPPKEFLERFKSLLQKMIHQHLSSRTHGSEDS | | |
| IL-21_5457 | N70G, K81D, | MRSSPGNMERIVICLMVIFLGTLVHKSSSQGQDRHMIRMRQ | 0.793 | 1166 |
| | H118D, T121E | LIDIVDQLKNYVNDLVPEFLPAPEDVETGCEWSAFSCFQDA | | |
| | | QLKSANTGNNERIINVSIKKLKRKPPSTNAGRRQKDRLECP | | |
| | | SCDSYEKKPPKEFLERFKSLLQKMIHQHLSSRTHGSEDS | | |
| IL-21_5445 | N70G, V98E, | MRSSPGNMERIVICLMVIFLGTLVHKSSSQGQDRHMIRMRQ | 0.793 | 1167 |
| | T110D, R114Q | LIDIVDQLKNYVNDLVPEFLPAPEDVETGCEWSAFSCFQKA | | |
| | | QLKSANTGNNERIINESIKKLKRKPPSDNAGQRQKHRLTCP | | |
| | | SCDSYEKKPPKEFLERFKSLLQKMIHQHLSSRTHGSEDS | | |
| IL-21_5173 | K81D, R114G, | MRSSPGNMERIVICLMVIFLGTLVHKSSSQGQDRHMIRMRQ | 0.793 | 1168 |
| | R115G, R119T | LIDIVDQLKNYVNDLVPEFLPAPEDVETNCEWSAFSCFQDA | | |
| | | QLKSANTGNNERIINVSIKKLKRKPPSTNAGGGQKHTLTCP | | |
| | | SCDSYEKKPPKEFLERFKSLLQKMIHQHLSSRTHGSEDS | | |
| IL-21_5222 | K81D, T110D, | MRSSPGNMERIVICLMVIFLGTLVHKSSSQGQDRHMIRMRQ | 0.793 | 1169 |
| | R114Q, H118D | LIDIVDQLKNYVNDLVPEFLPAPEDVETNCEWSAFSCFQDA | | |
| | | QLKSANTGNNERIINVSIKKLKRKPPSDNAGQRQKDRLTCP | | |
| | | SCDSYEKKPPKEFLERFKSLLQKMIHQHLSSRTHGSEDS | | |
| IL-21_5287 | K81D, V98E, | MRSSPGNMERIVICLMVIFLGTLVHKSSSQGQDRHMIRMRQ | 0.794 | 1170 |
| | A112P, H118D | LIDIVDQLKNYVNDLVPEFLPAPEDVETNCEWSAFSCFQDA | | |
| | | QLKSANTGNNERIINESIKKLKRKPPSTNPGRRQKDRLTCP | | |
| | | SCDSYEKKPPKEFLERFKSLLQKMIHQHLSSRTHGSEDS | | |
| IL-21_5893 | I45T, K81D, | MRSSPGNMERIVICLMVIFLGTLVHKSSSQGQDRHMIRMRQ | 0.797 | 1171 |
| | R114Q, R115D | LIDTVDQLKNYVNDLVPEFLPAPEDVETNCEWSAFSCFQDA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5277 | K81D, V98E, | MRSSPGNMERIVICLMVIFLGTLVHKSSSQGQDRHMIRMRQ | 0.797 | 1172 |
| | R114G, H118D | LIDIVDQLKNYVNDLVPEFLPAPEDVETNCEWSAFSCFQDA | | |
| | | QLKSANTGNNERIINESIKKLKRKPPSTNAGGRQKDRLTCP | | |
| | | SCDSYEKKPPKEFLERFKSLLQKMIHQHLSSRTHGSEDS | | |
| IL-21_5151 | V98E, P107V, | MRSSPGNMERIVICLMVIFLGTLVHKSSSQGQDRHMIRMRQ | 0.798 | 1173 |
| | A112P, R115D | LIDIVDQLKNYVNDLVPEFLPAPEDVETNCEWSAFSCFQKA | | |
| | | QLKSANTGNNERIINESIKKLKRKVPSTNPGRDQKHRLTCP | | |
| | | SCDSYEKKPPKEFLERFKSLLQKMIHQHLSSRTHGSEDS | | |
| IL-21_6305 | H35F, V98E, | MRSSPGNMERIVICLMVIFLGTLVHKSSSQGQDREMIRMRQ | 0.798 | 1174 |
| | R115D | LIDIVDQLKNYVNDLVPEFLPAPEDVETNCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5771 | N70G, K81D, | MRSSPGNMERIVICLMVIFLGTLVHKSSSQGQDRHMIRMRQ | 0.798 | 1175 |
| | V98E | LIDIVDQLKNYVNDLVPEFLPAPEDVETGCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5444 | N70G, V98E, | MRSSPGNMERIVICLMVIFLGTLVHKSSSQGQDRHMIRMRQ | 0.799 | 1176 |
| | T110D, R114G | LIDIVDQLKNYVNDLVPEFLPAPEDVETGCEWSAFSCFQKA | | |
| | | QLKSANTGNNERIINESIKKLKRKPPSDNAGGRQKHRLTCP | | |
| | | SCDSYEKKPPKEFLERFKSLLQKMIHQHLSSRTHGSEDS | | |
| IL-21_5468 | N70G, K81D, | MRSSPGNMERIVICLMVIFLGTLVHKSSSQGQDRHMIRMRQ | 0.799 | 1177 |
| | R114G, R115G | LIDIVDQLKNYVNDLVPEFLPAPEDVETGCEWSAFSCFQDA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5063 | V98E, R115D, | MRSSPGNMERIVICLMVIFLGTLVHKSSSQGQDRHMIRMRQ | 0.799 | 1178 |
| | H118D, T121E | LIDIVDQLKNYVNDLVPEFLPAPEDVETNCEWSAFSCFQKA | | |
| | | QLKSANTGNNERIINESIKKLKRKPPSTNAGRDQKDRLECP | | |
| | | SCDSYEKKPPKEFLERFKSLLQKMIHQHLSSRTHGSEDS | | |
| IL-21_5236 | K81D, P107V, | MRSSPGNMERIVICLMVIFLGTLVHKSSSQGQDRHMIRMRQ | 0.799 | 1179 |
| | R115G, R119T | LIDIVDQLKNYVNDLVPEFLPAPEDVETNCEWSAFSCFQDA | | |
| | | QLKSANTGNNERIINVSIKKLKRKVPSTNAGRGQKHTLTCP | | |
| | | SCDSYEKKPPKEFLERFKSLLQKMIHQHLSSRTHGSEDS | | |
| IL-21_6087 | H35F, I45T, | MRSSPGNMERIVICLMVIFLGTLVHKSSSQGQDRFMIRMRQ | 0.799 | 1180 |
| | K81D, V98E | LIDTVDQLKNYVNDLVPEFLPAPEDVETNCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5137 | V98E, P107V, | MRSSPGNMERIVICLMVIFLGTLVHKSSSQGQDRHMIRMRQ | 0.799 | 1181 |
| | R114G, T121E | LIDIVDQLKNYVNDLVPEFLPAPEDVETNCEWSAFSCFQKA | | |
| | | QLKSANTGNNERIINESIKKLKRKVPSTNAGGRQKHRLECP | | |
| | | SCDSYEKKPPKEFLERFKSLLQKMIHQHLSSRTHGSEDS | | |
| IL-21_5891 | I45T, K81D, | MRSSPGNMERIVICLMVIFLGTLVHKSSSQGQDRHMIRMRQ | 0.799 | 1182 |
| | R114G, R115D | LIDTVDQLKNYVNDLVPEFLPAPEDVETNCEWSAFSCFQDA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5112 | V98E, T110D, | MRSSPGNMERIVICLMVIFLGTLVHKSSSQGQDRHMIRMRQ | 0.801 | 1183 |
| | R114G, R119T | LIDIVDQLKNYVNDLVPEFLPAPEDVETNCEWSAFSCFQKA | | |
| | | QLKSANTGNNERIINESIKKLKRKPPSDNAGGRQKHTLTCP | | |
| | | SCDSYEKKPPKEFLERFKSLLQKMIHQHLSSRTHGSEDS | | |
| IL-21_6145 | H35I, K81D, | MRSSPGNMERIVICLMVIFLGTLVHKSSSQGQDRIMIRMRQ | 0.802 | 1184 |
| | A112P, R115G | LIDIVDQLKNYVNDLVPEFLPAPEDVETNCEWSAFSCFQDA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5094 | V98E, A112P, | MRSSPGNMERIVICLMVIFLGTLVHKSSSQGQDRHMIRMRQ | 0.804 | 1185 |
| | R114G, H118D | LIDIVDQLKNYVNDLVPEFLPAPEDVETNCEWSAFSCFQKA | | |
| | | QLKSANTGNNERIINESIKKLKRKPPSTNPGGRQKDRLTCP | | |
| | | SCDSYEKKPPKEFLERFKSLLQKMIHQHLSSRTHGSEDS | | |
| IL-21_5692 | K81D, P107V, | MRSSPGNMERIVICLMVIFLGTLVHKSSSQGQDRHMIRMRQ | 0.804 | 1186 |
| | R115G | LIDIVDQLKNYVNDLVPEFLPAPEDVETNCEWSAFSCFQDA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5102 | V98E, T110D, | MRSSPGNMERIVICLMVIFLGTLVHKSSSQGQDRHMIRMRQ | 0.806 | 1187 |
| | R119T, T121E | LIDIVDQLKNYVNDLVPEFLPAPEDVETNCEWSAFSCFQKA | | |
| | | QLKSANTGNNERIINESIKKLKRKPPSDNAGRRQKHTLECP | | |
| | | SCDSYEKKPPKEFLERFKSLLQKMIHQHLSSRTHGSEDS | | |
| IL-21_5965 | H35F, P107V, | MRSSPGNMERIVICLMVIFLGTLVHKSSSQGQDREMIRMRQ | 0.807 | 1188 |
| | R114Q, R115D | LIDIVDQLKNYVNDLVPEFLPAPEDVETNCEWSAFSCFQKA | | |
| | | QLKSANTGNNERIINVSIKKLKRKVPSTNAGQDQKHRLTCP | | |
| | | SCDSYEKKPPKEFLERFKSLLQKMIHQHLSSRTHGSEDS | | |
| IL-21_5461 | N70G, K81D, | MRSSPGNMERIVICLMVIFLGTLVHKSSSQGQDRHMIRMRQ | 0.807 | 1189 |
| | R115G, H118D | LIDIVDQLKNYVNDLVPEFLPAPEDVETGCEWSAFSCFQDA | | |
| | | QLKSANTGNNERIINVSIKKLKRKPPSTNAGRGQKDRLTCP | | |
| | | SCDSYEKKPPKEFLERFKSLLQKMIHQHLSSRTHGSEDS | | |
| IL-21_5068 | V98E, R114G, | MRSSPGNMERIVICLMVIFLGTLVHKSSSQGQDRHMIRMRQ | 0.807 | 1190 |
| | R115G, T121E | LIDIVDQLKNYVNDLVPEFLPAPEDVETNCEWSAFSCFQKA | | |
| | | QLKSANTGNNERIINESIKKLKRKPPSTNAGGGQKHRLECP | | |
| | | SCDSYEKKPPKEFLERFKSLLQKMIHQHLSSRTHGSEDS | | |
| IL-21_5085 | V98E, A112P, | MRSSPGNMERIVICLMVIFLGTLVHKSSSQGQDRHMIRMRQ | 0.808 | 1191 |
| | H118D, R119T | LIDIVDQLKNYVNDLVPEFLPAPEDVETNCEWSAFSCFQKA | | |
| | | QLKSANTGNNERIINESIKKLKRKPPSTNPGRRQKDTLTCP | | |
| | | SCDSYEKKPPKEFLERFKSLLQKMIHQHLSSRTHGSEDS | | |
| IL-21_5765 | N70G, K81D, | MRSSPGNMERIVICLMVIFLGTLVHKSSSQGQDRHMIRMRQ | 0.808 | 1192 |
| | R115D | LIDIVDQLKNYVNDLVPEFLPAPEDVETGCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_6040 | H35F, N70G, | MRSSPGNMERIVICLMVIFLGTLVHKSSSQGQDREMIRMRQ | 0.809 | 1193 |
| | V98E, R115D | LIDIVDQLKNYVNDLVPEFLPAPEDVETGCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_6083 | H35F, I45T, | MRSSPGNMERIVICLMVIFLGTLVHKSSSQGQDRFMIRMRQ | 0.809 | 1194 |
| | K81D, R114Q | LIDTVDQLKNYVNDLVPEFLPAPEDVETNCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5685 | K81D, T110D, | MRSSPGNMERIVICLMVIFLGTLVHKSSSQGQDRHMIRMRQ | 0.809 | 1195 |
| | R115D | LIDIVDQLKNYVNDLVPEFLPAPEDVETNCEWSAFSCFQDA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6227 | H35I, I45T, | MRSSPGNMERIVICLMVIFLGTLVHKSSSQGQDRIMIRMRQ | 0.81 | 1196 |
| | K81D, R115D | LIDTVDQLKNYVNDLVPEFLPAPEDVETNCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5176 | K81D, R114G, | MRSSPGNMERIVICLMVIFLGTLVHKSSSQGQDRHMIRMRQ | 0.81 | 1197 |
| | R115D, R119T | LIDIVDQLKNYVNDLVPEFLPAPEDVETNCEWSAFSCFQDA | | |
| | | QLKSANTGNNERIINVSIKKLKRKPPSTNAGGDQKHTLTCP | | |
| | | SCDSYEKKPPKEFLERFKSLLQKMIHQHLSSRTHGSEDS | | |
| IL-21_5124 | V98E, T110D, | MRSSPGNMERIVICLMVIFLGTLVHKSSSQGQDRHMIRMRQ | 0.81 | 1198 |
| | A112P, R115G | LIDIVDQLKNYVNDLVPEFLPAPEDVETNCEWSAFSCFQKA | | |
| | | QLKSANTGNNERIINESIKKLKRKPPSDNPGRGQKHRLTCP | | |
| | | SCDSYEKKPPKEFLERFKSLLQKMIHQHLSSRTHGSEDS | | |
| IL-21_5205 | K81D, A112P, | MRSSPGNMERIVICLMVIFLGTLVHKSSSQGQDRHMIRMRQ | 0.81 | 1199 |
| | R114Q, R115D | LIDIVDQLKNYVNDLVPEFLPAPEDVETNCEWSAFSCFQDA | | |
| | | QLKSANTGNNERIINVSIKKLKRKPPSTNPGQDQKHRLTCP | | |
| | | SCDSYEKKPPKEFLERFKSLLQKMIHQHLSSRTHGSEDS | | |
| IL-21_5090 | V98E, A112P, | MRSSPGNMERIVICLMVIFLGTLVHKSSSQGQDRHMIRMRQ | 0.81 | 1200 |
| | R115D, R119T | LIDIVDQLKNYVNDLVPEFLPAPEDVETNCEWSAFSCFQKA | | |
| | | QLKSANTGNNERIINESIKKLKRKPPSTNPGRDQKHTLTCP | | |
| | | SCDSYEKKPPKEFLERFKSLLQKMIHQHLSSRTHGSEDS | | |
| IL-21_5818 | V98E, R115D | MRSSPGNMERIVICLMVIFLGTLVHKSSSQGQDRHMIRMRQ | 0.811 | 1201 |
| | | LIDIVDQLKNYVNDLVPEFLPAPEDVETNCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5207 | K81D, T110D, | MRSSPGNMERIVICLMVIFLGTLVHKSSSQGQDRHMIRMRQ | 0.812 | 1202 |
| | H118D, T121E | LIDIVDQLKNYVNDLVPEFLPAPEDVETNCEWSAFSCFQDA | | |
| | | QLKSANTGNNERIINVSIKKLKRKPPSDNAGRRQKDRLECP | | |
| | | SCDSYEKKPPKEFLERFKSLLQKMIHQHLSSRTHGSEDS | | |
| IL-21_5880 | I45T, V98E, | MRSSPGNMERIVICLMVIFLGTLVHKSSSQGQDRHMIRMRQ | 0.815 | 1203 |
| | T110D, R115D | LIDTVDQLKNYVNDLVPEFLPAPEDVETNCEWSAFSCFQKA | | |
| | | QLKSANTGNNERIINESIKKLKRKPPSDNAGRDQKHRLTCP | | |
| | | SCDSYEKKPPKEFLERFKSLLQKMIHQHLSSRTHGSEDS | | |
| IL-21_5138 | V98E, P107V, | MRSSPGNMERIVICLMVIFLGTLVHKSSSQGQDRHMIRMRQ | 0.815 | 1204 |
| | R114G, R119T | LIDIVDQLKNYVNDLVPEFLPAPEDVETNCEWSAFSCFQKA | | |
| | | QLKSANTGNNERIINESIKKLKRKVPSTNAGGRQKHTLTCP | | |
| | | SCDSYEKKPPKEFLERFKSLLQKMIHQHLSSRTHGSEDS | | |
| IL-21_6188 | H35I, N70G, | MRSSPGNMERIVICLMVIFLGTLVHKSSSQGQDRIMIRMRQ | 0.817 | 1205 |
| | V98E, R114Q | LIDIVDQLKNYVNDLVPEFLPAPEDVETGCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_6041 | H35F, N70G, | MRSSPGNMERIVICLMVIFLGTLVHKSSSQGQDRFMIRMRQ | 0.817 | 1206 |
| | V98E, R114G | LIDIVDQLKNYVNDLVPEFLPAPEDVETGCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5100 | V98E, A112P, | MRSSPGNMERIVICLMVIFLGTLVHKSSSQGQDRHMIRMRQ | 0.818 | 1207 |
| | R114Q, R115G | LIDIVDQLKNYVNDLVPEFLPAPEDVETNCEWSAFSCFQKA | | |
| | | QLKSANTGNNERIINESIKKLKRKPPSTNPGQGQKHRLTCP | | |
| | | SCDSYEKKPPKEFLERFKSLLQKMIHQHLSSRTHGSEDS | | |
| IL-21_5058 | V98E, H118D, | MRSSPGNMERIVICLMVIFLGTLVHKSSSQGQDRHMIRMRQ | 0.818 | 1208 |
| | R119T, T121E | LIDIVDQLKNYVNDLVPEFLPAPEDVETNCEWSAFSCFQKA | | |
| | | QLKSANTGNNERIINESIKKLKRKPPSTNAGRRQKDTLECP | | |
| | | SCDSYEKKPPKEFLERFKSLLQKMIHQHLSSRTHGSEDS | | |
| IL-21_5232 | K81D, P107V, | MRSSPGNMERIVICLMVIFLGTLVHKSSSQGQDRHMIRMRQ | 0.819 | 1209 |
| | R119T, T121E | LIDIVDQLKNYVNDLVPEFLPAPEDVETNCEWSAFSCFQDA | | |
| | | QLKSANTGNNERIINVSIKKLKRKVPSTNAGRRQKHTLECP | | |
| | | SCDSYEKKPPKEFLERFKSLLQKMIHQHLSSRTHGSEDS | | |
| IL-21_5628 | V98E, R114Q, | MRSSPGNMERIVICLMVIFLGTLVHKSSSQGQDRHMIRMRQ | 0.82 | 1210 |
| | H118D | LIDIVDQLKNYVNDLVPEFLPAPEDVETNCEWSAFSCFQKA | | |
| | | QLKSANTGNNERIINESIKKLKRKPPSTNAGQRQKDRLTCP | | |
| | | SCDSYEKKPPKEFLERFKSLLQKMIHQHLSSRTHGSEDS | | |
| IL-21_6159 | H35I, K81D, | MRSSPGNMERIVICLMVIFLGTLVHKSSSQGQDRIMIRMRQ | 0.82 | 1211 |
| | P107V, T110D | LIDIVDQLKNYVNDLVPEFLPAPEDVETNCEWSAFSCFQDA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5756 | N70G, V98E, | MRSSPGNMERIVICLMVIFLGTLVHKSSSQGQDRHMIRMRQ | 0.82 | 1212 |
| | R114G | LIDIVDQLKNYVNDLVPEFLPAPEDVETGCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5162 | K81D, H118D, | MRSSPGNMERIVICLMVIFLGTLVHKSSSQGQDRHMIRMRQ | 0.82 | 1213 |
| | R119T, T121E | LIDIVDQLKNYVNDLVPEFLPAPEDVETNCEWSAFSCFQDA | | |
| | | QLKSANTGNNERIINVSIKKLKRKPPSTNAGRRQKDTLECP | | |
| | | SCDSYEKKPPKEFLERFKSLLQKMIHQHLSSRTHGSEDS | | |
| IL-21_5957 | H35F, T110D, | MRSSPGNMERIVICLMVIFLGTLVHKSSSQGQDREMIRMRQ | 0.821 | 1214 |
| | R114Q, R115D | LIDIVDQLKNYVNDLVPEFLPAPEDVETNCEWSAFSCFQKA | | |
| | | QLKSANTGNNERIINVSIKKLKRKPPSDNAGQDQKHRLTCP | | |
| | | SCDSYEKKPPKEFLERFKSLLQKMIHQHLSSRTHGSEDS | | |
| IL-21_5268 | K81D, V98E, | MRSSPGNMERIVICLMVIFLGTLVHKSSSQGQDRHMIRMRQ | 0.821 | 1215 |
| | H118D, R119T | LIDIVDQLKNYVNDLVPEFLPAPEDVETNCEWSAFSCFQDA | | |
| | | QLKSANTGNNERIINESIKKLKRKPPSTNAGRRQKDTLTCP | | |
| | | SCDSYEKKPPKEFLERFKSLLQKMIHQHLSSRTHGSEDS | | |
| IL-21_5210 | K81D, T110D, | MRSSPGNMERIVICLMVIFLGTLVHKSSSQGQDRHMIRMRQ | 0.822 | 1216 |
| | R115G, R119T | LIDIVDQLKNYVNDLVPEFLPAPEDVETNCEWSAFSCFQDA | | |
| | | QLKSANTGNNERIINVSIKKLKRKPPSDNAGRGQKHTLTCP | | |
| | | SCDSYEKKPPKEFLERFKSLLQKMIHQHLSSRTHGSEDS | | |
| IL-21_5417 | N70G, V98E, | MRSSPGNMERIVICLMVIFLGTLVHKSSSQGQDRHMIRMRQ | 0.822 | 1217 |
| | R115G, R119T | LIDIVDQLKNYVNDLVPEFLPAPEDVETGCEWSAFSCFQKA | | |
| | | QLKSANTGNNERIINESIKKLKRKPPSTNAGRGQKHTLTCP | | |
| | | SCDSYEKKPPKEFLERFKSLLQKMIHQHLSSRTHGSEDS | | |
| IL-21_5662 | K81D, R115D, | MRSSPGNMERIVICLMVIFLGTLVHKSSSQGQDRHMIRMRQ | 0.822 | 1218 |
| | R119T | LIDIVDQLKNYVNDLVPEFLPAPEDVETNCEWSAFSCFQDA | | |
| | | QLKSANTGNNERIINVSIKKLKRKPPSTNAGRDQKHTLTCP | | |
| | | SCDSYEKKPPKEFLERFKSLLQKMIHQHLSSRTHGSEDS | | |
| IL-21_5065 | V98E, R114G, | MRSSPGNMERIVICLMVIFLGTLVHKSSSQGQDRHMIRMRQ | 0.823 | 1219 |
| | R119T, T121E | LIDIVDQLKNYVNDLVPEFLPAPEDVETNCEWSAFSCFQKA | | |
| | | QLKSANTGNNERIINESIKKLKRKPPSTNAGGRQKHTLECP | | |
| | | SCDSYEKKPPKEFLERFKSLLQKMIHQHLSSRTHGSEDS | | |
| IL-21_5650 | V98E, P107V, | MRSSPGNMERIVICLMVIFLGTLVHKSSSQGQDRHMIRMRQ | 0.824 | 1220 |
| | R115D | LIDIVDQLKNYVNDLVPEFLPAPEDVETNCEWSAFSCFQKA | | |
| | | QLKSANTGNNERIINESIKKLKRKVPSTNAGRDQKHRLTCP | | |
| | | SCDSYEKKPPKEFLERFKSLLQKMIHQHLSSRTHGSEDS | | |
| IL-21_5496 | N70G, K81D, | MRSSPGNMERIVICLMVIFLGTLVHKSSSQGQDRHMIRMRQ | 0.825 | 1221 |
| | P107V, R114Q | LIDIVDQLKNYVNDLVPEFLPAPEDVETGCEWSAFSCFQDA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5661 | K81D, R115D, | MRSSPGNMERIVICLMVIFLGTLVHKSSSQGQDRHMIRMRQ | 0.826 | 1222 |
| | T121E | LIDIVDQLKNYVNDLVPEFLPAPEDVETNCEWSAFSCFQDA | | |
| | | QLKSANTGNNERIINVSIKKLKRKPPSTNAGRDQKHRLECP | | |
| | | SCDSYEKKPPKEFLERFKSLLQKMIHQHLSSRTHGSEDS | | |
| IL-21_6365 | H35I, K81D, | MRSSPGNMERIVICLMVIFLGTLVHKSSSQGQDRIMIRMRQ | 0.829 | 1223 |
| | R115D | LIDIVDQLKNYVNDLVPEFLPAPEDVETNCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5266 | K81D, V98E, | MRSSPGNMERIVICLMVIFLGTLVHKSSSQGQDRHMIRMRQ | 0.83 | 1224 |
| | R119T, T121E | LIDIVDQLKNYVNDLVPEFLPAPEDVETNCEWSAFSCFQDA | | |
| | | QLKSANTGNNERIINESIKKLKRKPPSTNAGRRQKHTLECP | | |
| | | SCDSYEKKPPKEFLERFKSLLQKMIHQHLSSRTHGSEDS | | |
| IL-21_5265 | K81D, P107V, | MRSSPGNMERIVICLMVIFLGTLVHKSSSQGQDRHMIRMRQ | 0.83 | 1225 |
| | T110D, A112P | LIDIVDQLKNYVNDLVPEFLPAPEDVETNCEWSAFSCFQDA | | |
| | | QLKSANTGNNERIINVSIKKLKRKVPSDNPGRRQKHRLTCP | | |
| | | SCDSYEKKPPKEFLERFKSLLQKMIHQHLSSRTHGSEDS | | |
| IL-21_6128 | H35I, V98E, | MRSSPGNMERIVICLMVIFLGTLVHKSSSQGQDRIMIRMRQ | 0.83 | 1226 |
| | A112P, R114G | LIDIVDQLKNYVNDLVPEFLPAPEDVETNCEWSAFSCFQKA | | |
| | | QLKSANTGNNERIINESIKKLKRKPPSTNPGGRQKHRLTCP | | |
| | | SCDSYEKKPPKEFLERFKSLLQKMIHQHLSSRTHGSEDS | | |
| IL-21_5619 | V98E, R115D, | MRSSPGNMERIVICLMVIFLGTLVHKSSSQGQDRHMIRMRQ | 0.831 | 1227 |
| | R119T | LIDIVDQLKNYVNDLVPEFLPAPEDVETNCEWSAFSCFQKA | | |
| | | QLKSANTGNNERIINESIKKLKRKPPSTNAGRDQKHTLTCP | | |
| | | SCDSYEKKPPKEFLERFKSLLQKMIHQHLSSRTHGSEDS | | |
| IL-21_5492 | N70G, K81D, | MRSSPGNMERIVICLMVIFLGTLVHKSSSQGQDRHMIRMRQ | 0.831 | 1228 |
| | P107V, H118D | LIDIVDQLKNYVNDLVPEFLPAPEDVETGCEWSAFSCFQDA | | |
| | | QLKSANTGNNERIINVSIKKLKRKVPSTNAGRRQKDRLTCP | | |
| | | SCDSYEKKPPKEFLERFKSLLQKMIHQHLSSRTHGSEDS | | |
| IL-21_5218 | K81D, T110D, | MRSSPGNMERIVICLMVIFLGTLVHKSSSQGQDRHMIRMRQ | 0.832 | 1229 |
| | R114G, R115G | LIDIVDQLKNYVNDLVPEFLPAPEDVETNCEWSAFSCFQDA | | |
| | | QLKSANTGNNERIINVSIKKLKRKPPSDNAGGGQKHRLTCP | | |
| | | SCDSYEKKPPKEFLERFKSLLQKMIHQHLSSRTHGSEDS | | |
| IL-21_5061 | V98E, R115G, | MRSSPGNMERIVICLMVIFLGTLVHKSSSQGQDRHMIRMRQ | 0.832 | 1230 |
| | H118D, R119T | LIDIVDQLKNYVNDLVPEFLPAPEDVETNCEWSAFSCFQKA | | |
| | | QLKSANTGNNERIINESIKKLKRKPPSTNAGRGQKDTLTCP | | |
| | | SCDSYEKKPPKEFLERFKSLLQKMIHQHLSSRTHGSEDS | | |
| IL-21_5660 | K81D, R115G, | MRSSPGNMERIVICLMVIFLGTLVHKSSSQGQDRHMIRMRQ | 0.833 | 1231 |
| | H118D | LIDIVDQLKNYVNDLVPEFLPAPEDVETNCEWSAFSCFQDA | | |
| | | QLKSANTGNNERIINVSIKKLKRKPPSTNAGRGQKDRLTCP | | |
| | | SCDSYEKKPPKEFLERFKSLLQKMIHQHLSSRTHGSEDS | | |
| IL-21_6313 | H35F, K81D, | MRSSPGNMERIVICLMVIFLGTLVHKSSSQGQDREMIRMRQ | 0.833 | 1232 |
| | R114G | LIDIVDQLKNYVNDLVPEFLPAPEDVETNCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_6193 | H35I, N70G, | MRSSPGNMERIVICLMVIFLGTLVHKSSSQGQDRIMIRMRQ | 0.835 | 1233 |
| | K81D, R115D | LIDIVDQLKNYVNDLVPEFLPAPEDVETGCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_6080 | H35F, I45T, | MRSSPGNMERIVICLMVIFLGTLVHKSSSQGQDREMIRMRQ | 0.836 | 1234 |
| | K81D, R115G | LIDTVDQLKNYVNDLVPEFLPAPEDVETNCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5169 | K81D, R114G, | MRSSPGNMERIVICLMVIFLGTLVHKSSSQGQDRHMIRMRQ | 0.836 | 1235 |
| | R119T, T121E | LIDIVDQLKNYVNDLVPEFLPAPEDVETNCEWSAFSCFQDA | | |
| | | QLKSANTGNNERIINVSIKKLKRKPPSTNAGGRQKHTLECP | | |
| | | SCDSYEKKPPKEFLERFKSLLQKMIHQHLSSRTHGSEDS | | |
| IL-21_5434 | N70G, V98E, | MRSSPGNMERIVICLMVIFLGTLVHKSSSQGQDRHMIRMRQ | 0.837 | 1236 |
| | A112P, H118D | LIDIVDQLKNYVNDLVPEFLPAPEDVETGCEWSAFSCFQKA | | |
| | | QLKSANTGNNERIINESIKKLKRKPPSTNPGRRQKDRLTCP | | |
| | | SCDSYEKKPPKEFLERFKSLLQKMIHQHLSSRTHGSEDS | | |
| IL-21_5371 | N70G, T110D, | MRSSPGNMERIVICLMVIFLGTLVHKSSSQGQDRHMIRMRQ | 0.837 | 1237 |
| | R114Q, R115D | LIDIVDQLKNYVNDLVPEFLPAPEDVETGCEWSAFSCFQKA | | |
| | | QLKSANTGNNERIINVSIKKLKRKPPSDNAGQDQKHRLTCP | | |
| | | SCDSYEKKPPKEFLERFKSLLQKMIHQHLSSRTHGSEDS | | |
| IL-21_5458 | N70G, K81D, | MRSSPGNMERIVICLMVIFLGTLVHKSSSQGQDRHMIRMRQ | 0.837 | 1238 |
| | H118D, R119T | LIDIVDQLKNYVNDLVPEFLPAPEDVETGCEWSAFSCFQDA | | |
| | | QLKSANTGNNERIINVSIKKLKRKPPSTNAGRRQKDTLTCP | | |
| | | SCDSYEKKPPKEFLERFKSLLQKMIHQHLSSRTHGSEDS | | |
| IL-21_5700 | K81D, V98E, | MRSSPGNMERIVICLMVIFLGTLVHKSSSQGQDRHMIRMRQ | 0.837 | 1239 |
| | H118D | LIDIVDQLKNYVNDLVPEFLPAPEDVETNCEWSAFSCFQDA | | |
| | | QLKSANTGNNERIINESIKKLKRKPPSTNAGRRQKDRLTCP | | |
| | | SCDSYEKKPPKEFLERFKSLLQKMIHQHLSSRTHGSEDS | | |
| IL-21_6050 | H35F, N70G, | MRSSPGNMERIVICLMVIFLGTLVHKSSSQGQDRFMIRMRQ | 0.838 | 1240 |
| | K81D, A112P | LIDIVDQLKNYVNDLVPEFLPAPEDVETGCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5111 | V98E, T110D, | MRSSPGNMERIVICLMVIFLGTLVHKSSSQGQDRHMIRMRQ | 0.838 | 1241 |
| | R114G, T121E | LIDIVDQLKNYVNDLVPEFLPAPEDVETNCEWSAFSCFQKA | | |
| | | QLKSANTGNNERIINESIKKLKRKPPSDNAGGRQKHRLECP | | |
| | | SCDSYEKKPPKEFLERFKSLLQKMIHQHLSSRTHGSEDS | | |
| IL-21_5698 | K81D, V98E, | MRSSPGNMERIVICLMVIFLGTLVHKSSSQGQDRHMIRMRQ | 0.839 | 1242 |
| | T121E | LIDIVDQLKNYVNDLVPEFLPAPEDVETNCEWSAFSCFQDA | | |
| | | QLKSANTGNNERIINESIKKLKRKPPSTNAGRRQKHRLECP | | |
| | | SCDSYEKKPPKEFLERFKSLLQKMIHQHLSSRTHGSEDS | | |
| IL-21_6052 | H35F, N70G, | MRSSPGNMERIVICLMVIFLGTLVHKSSSQGQDRFMIRMRQ | 0.84 | 1243 |
| | K81D, P107V | LIDIVDQLKNYVNDLVPEFLPAPEDVETGCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5089 | V98E, A112P, | MRSSPGNMERIVICLMVIFLGTLVHKSSSQGQDRHMIRMRQ | 0.84 | 1244 |
| | R115D, T121E | LIDIVDQLKNYVNDLVPEFLPAPEDVETNCEWSAFSCFQKA | | |
| | | QLKSANTGNNERIINESIKKLKRKPPSTNPGRDQKHRLECP | | |
| | | SCDSYEKKPPKEFLERFKSLLQKMIHQHLSSRTHGSEDS | | |
| IL-21_5174 | K81D, R114G, | MRSSPGNMERIVICLMVIFLGTLVHKSSSQGQDRHMIRMRQ | 0.84 | 1245 |
| | R115G, H118D | LIDIVDQLKNYVNDLVPEFLPAPEDVETNCEWSAFSCFQDA | | |
| | | QLKSANTGNNERIINVSIKKLKRKPPSTNAGGGQKDRLTCP | | |
| | | SCDSYEKKPPKEFLERFKSLLQKMIHQHLSSRTHGSEDS | | |
| IL-21_5366 | N70G, T110D, | MRSSPGNMERIVICLMVIFLGTLVHKSSSQGQDRHMIRMRQ | 0.842 | 1246 |
| | R114G, R115D | LIDIVDQLKNYVNDLVPEFLPAPEDVETGCEWSAFSCFQKA | | |
| | | QLKSANTGNNERIINVSIKKLKRKPPSDNAGGDQKHRLTCP | | |
| | | SCDSYEKKPPKEFLERFKSLLQKMIHQHLSSRTHGSEDS | | |
| IL-21_5479 | N70G, K81D, | MRSSPGNMERIVICLMVIFLGTLVHKSSSQGQDRHMIRMRQ | 0.842 | 1247 |
| | A112P, R115D | LIDIVDQLKNYVNDLVPEFLPAPEDVETGCEWSAFSCFQDA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5874 | I45T, V98E, | MRSSPGNMERIVICLMVIFLGTLVHKSSSQGQDRHMIRMRQ | 0.843 | 1248 |
| | R114Q, R115D | LIDTVDQLKNYVNDLVPEFLPAPEDVETNCEWSAFSCFQKA | | |
| | | QLKSANTGNNERIINESIKKLKRKPPSTNAGQDQKHRLTCP | | |
| | | SCDSYEKKPPKEFLERFKSLLQKMIHQHLSSRTHGSEDS | | |
| IL-21_6157 | H35I, K81D, | MRSSPGNMERIVICLMVIFLGTLVHKSSSQGQDRIMIRMRQ | 0.843 | 1249 |
| | P107V, R114Q | LIDIVDQLKNYVNDLVPEFLPAPEDVETNCEWSAFSCFQDA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_6039 | H35F, N70G, | MRSSPGNMERIVICLMVIFLGTLVHKSSSQGQDREMIRMRQ | 0.844 | 1250 |
| | V98E, R115G | LIDIVDQLKNYVNDLVPEFLPAPEDVETGCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_5450 | N70G, V98E, | MRSSPGNMERIVICLMVIFLGTLVHKSSSQGQDRHMIRMRQ | 0.845 | 1251 |
| | P107V, R115G | LIDIVDQLKNYVNDLVPEFLPAPEDVETGCEWSAFSCFQKA | | |
| | | QLKSANTGNNERIINESIKKLKRKVPSTNAGRGQKHRLTCP | | |
| | | SCDSYEKKPPKEFLERFKSLLQKMIHQHLSSRTHGSEDS | | |
| IL-21_5198 | K81D, A112P, | MRSSPGNMERIVICLMVIFLGTLVHKSSSQGQDRHMIRMRQ | 0.847 | 1252 |
| | R114G, H118D | LIDIVDQLKNYVNDLVPEFLPAPEDVETNCEWSAFSCFQDA | | |
| | | QLKSANTGNNERIINVSIKKLKRKPPSTNPGGRQKDRLTCP | | |
| | | SCDSYEKKPPKEFLERFKSLLQKMIHQHLSSRTHGSEDS | | |
| IL-21_5828 | K81D, R115D | MRSSPGNMERIVICLMVIFLGTLVHKSSSQGQDRHMIRMRQ | 0.847 | 1253 |
| | | LIDIVDQLKNYVNDLVPEFLPAPEDVETNCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5293 | K81D, V98E, | MRSSPGNMERIVICLMVIFLGTLVHKSSSQGQDRHMIRMRQ | 0.849 | 1254 |
| | T110D, R119T | LIDIVDQLKNYVNDLVPEFLPAPEDVETNCEWSAFSCFQDA | | |
| | | QLKSANTGNNERIINESIKKLKRKPPSDNAGRRQKHTLTCP | | |
| | | SCDSYEKKPPKEFLERFKSLLQKMIHQHLSSRTHGSEDS | | |
| IL-21_5276 | K81D, V98E, | MRSSPGNMERIVICLMVIFLGTLVHKSSSQGQDRHMIRMRQ | 0.849 | 1255 |
| | R114G, R119T | LIDIVDQLKNYVNDLVPEFLPAPEDVETNCEWSAFSCFQDA | | |
| | | QLKSANTGNNERIINESIKKLKRKPPSTNAGGRQKHTLTCP | | |
| | | SCDSYEKKPPKEFLERFKSLLQKMIHQHLSSRTHGSEDS | | |
| IL-21_5220 | K81D, T110D, | MRSSPGNMERIVICLMVIFLGTLVHKSSSQGQDRHMIRMRQ | 0.85 | 1256 |
| | R114Q, T121E | LIDIVDQLKNYVNDLVPEFLPAPEDVETNCEWSAFSCFQDA | | |
| | | QLKSANTGNNERIINVSIKKLKRKPPSDNAGQRQKHRLECP | | |
| | | SCDSYEKKPPKEFLERFKSLLQKMIHQHLSSRTHGSEDS | | |
| IL-21_6152 | H35I, K81D, | MRSSPGNMERIVICLMVIFLGTLVHKSSSQGQDRIMIRMRQ | 0.85 | 1257 |
| | T110D, R114Q | LIDIVDQLKNYVNDLVPEFLPAPEDVETNCEWSAFSCFQDA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_6141 | H35I, K81D, | MRSSPGNMERIVICLMVIFLGTLVHKSSSQGQDRIMIRMRQ | 0.851 | 1258 |
| | R114G, R115G | LIDIVDQLKNYVNDLVPEFLPAPEDVETNCEWSAFSCFQDA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_6012 | H35F, K81D, | MRSSPGNMERIVICLMVIFLGTLVHKSSSQGQDRFMIRMRQ | 0.852 | 1259 |
| | P107V, T110D | LIDIVDQLKNYVNDLVPEFLPAPEDVETNCEWSAFSCFQDA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5215 | K81D, T110D, | MRSSPGNMERIVICLMVIFLGTLVHKSSSQGQDRHMIRMRQ | 0.853 | 1260 |
| | R114G, T121E | LIDIVDQLKNYVNDLVPEFLPAPEDVETNCEWSAFSCFQDA | | |
| | | QLKSANTGNNERIINVSIKKLKRKPPSDNAGGRQKHRLECP | | |
| | | SCDSYEKKPPKEFLERFKSLLQKMIHQHLSSRTHGSEDS | | |
| IL-21_5285 | K81D, V98E, | MRSSPGNMERIVICLMVIFLGTLVHKSSSQGQDRHMIRMRQ | 0.853 | 1261 |
| | A112P, T121E | LIDIVDQLKNYVNDLVPEFLPAPEDVETNCEWSAFSCFQDA | | |
| | | QLKSANTGNNERIINESIKKLKRKPPSTNPGRRQKHRLECP | | |
| | | SCDSYEKKPPKEFLERFKSLLQKMIHQHLSSRTHGSEDS | | |
| IL-21_5955 | H35F, T110D, | MRSSPGNMERIVICLMVIFLGTLVHKSSSQGQDREMIRMRQ | 0.854 | 1262 |
| | R114G, R115D | LIDIVDQLKNYVNDLVPEFLPAPEDVETNCEWSAFSCFQKA | | |
| | | QLKSANTGNNERIINVSIKKLKRKPPSDNAGGDQKHRLTCP | | |
| | | SCDSYEKKPPKEFLERFKSLLQKMIHQHLSSRTHGSEDS | | |
| IL-21_5640 | V98E, T110D, | MRSSPGNMERIVICLMVIFLGTLVHKSSSQGQDRHMIRMRQ | 0.855 | 1263 |
| | H118D | LIDIVDQLKNYVNDLVPEFLPAPEDVETNCEWSAFSCFQKA | | |
| | | QLKSANTGNNERIINESIKKLKRKPPSDNAGRRQKDRLTCP | | |
| | | SCDSYEKKPPKEFLERFKSLLQKMIHQHLSSRTHGSEDS | | |
| IL-21_5898 | I45T, K81D, | MRSSPGNMERIVICLMVIFLGTLVHKSSSQGQDRHMIRMRQ | 0.856 | 1264 |
| | T110D, R115G | LIDTVDQLKNYVNDLVPEFLPAPEDVETNCEWSAFSCFQDA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_5286 | K81D, V98E, | MRSSPGNMERIVICLMVIFLGTLVHKSSSQGQDRHMIRMRQ | 0.857 | 1265 |
| | A112P, R119T | LIDIVDQLKNYVNDLVPEFLPAPEDVETNCEWSAFSCFQDA | | |
| | | QLKSANTGNNERIINESIKKLKRKPPSTNPGRRQKHTLTCP | | |
| | | SCDSYEKKPPKEFLERFKSLLQKMIHQHLSSRTHGSEDS | | |
| IL-21_5484 | N70G, K81D, | MRSSPGNMERIVICLMVIFLGTLVHKSSSQGQDRHMIRMRQ | 0.858 | 1266 |
| | T110D, H118D | LIDIVDQLKNYVNDLVPEFLPAPEDVETGCEWSAFSCFQDA | | |
| | | QLKSANTGNNERIINVSIKKLKRKPPSDNAGRRQKDRLTCP | | |
| | | SCDSYEKKPPKEFLERFKSLLQKMIHQHLSSRTHGSEDS | | |
| IL-21_5168 | K81D, R115D, | MRSSPGNMERIVICLMVIFLGTLVHKSSSQGQDRHMIRMRQ | 0.859 | 1267 |
| | H118D, R119T | LIDIVDQLKNYVNDLVPEFLPAPEDVETNCEWSAFSCFQDA | | |
| | | QLKSANTGNNERIINVSIKKLKRKPPSTNAGRDQKDTLTCP | | |
| | | SCDSYEKKPPKEFLERFKSLLQKMIHQHLSSRTHGSEDS | | |
| IL-21_6149 | H35I, K81D, | MRSSPGNMERIVICLMVIFLGTLVHKSSSQGQDRIMIRMRQ | 0.859 | 1268 |
| | T110D, R115G | LIDIVDQLKNYVNDLVPEFLPAPEDVETNCEWSAFSCFQDA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_6042 | H35F, N70G, | MRSSPGNMERIVICLMVIFLGTLVHKSSSQGQDRFMIRMRQ | 0.86 | 1269 |
| | V98E, R114Q | LIDIVDQLKNYVNDLVPEFLPAPEDVETGCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_6316 | H35F, K81D, | MRSSPGNMERIVICLMVIFLGTLVHKSSSQGQDREMIRMRQ | 0.86 | 1270 |
| | T110D | LIDIVDQLKNYVNDLVPEFLPAPEDVETNCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_6228 | H35I, I45T, | MRSSPGNMERIVICLMVIFLGTLVHKSSSQGQDRIMIRMRQ | 0.862 | 1271 |
| | K81D, R114G | LIDTVDQLKNYVNDLVPEFLPAPEDVETNCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5460 | N70G, K81D, | MRSSPGNMERIVICLMVIFLGTLVHKSSSQGQDRHMIRMRQ | 0.862 | 1272 |
| | R115G, R119T | LIDIVDQLKNYVNDLVPEFLPAPEDVETGCEWSAFSCFQDA | | |
| | | QLKSANTGNNERIINVSIKKLKRKPPSTNAGRGQKHTLTCP | | |
| | | SCDSYEKKPPKEFLERFKSLLQKMIHQHLSSRTHGSEDS | | |
| IL-21_5643 | V98E, T110D, | MRSSPGNMERIVICLMVIFLGTLVHKSSSQGQDRHMIRMRQ | 0.862 | 1273 |
| | R114G | LIDIVDQLKNYVNDLVPEFLPAPEDVETNCEWSAFSCFQKA | | |
| | | QLKSANTGNNERIINESIKKLKRKPPSDNAGGRQKHRLTCP | | |
| | | SCDSYEKKPPKEFLERFKSLLQKMIHQHLSSRTHGSEDS | | |
| IL-21_5975 | H35F, V98E, | MRSSPGNMERIVICLMVIFLGTLVHKSSSQGQDRFMIRMRQ | 0.863 | 1274 |
| | R114G, R115G | LIDIVDQLKNYVNDLVPEFLPAPEDVETNCEWSAFSCFQKA | | |
| | | QLKSANTGNNERIINESIKKLKRKPPSTNAGGGQKHRLTCP | | |
| | | SCDSYEKKPPKEFLERFKSLLQKMIHQHLSSRTHGSEDS | | |
| IL-21_5633 | V98E, A112P, | MRSSPGNMERIVICLMVIFLGTLVHKSSSQGQDRHMIRMRQ | 0.863 | 1275 |
| | H118D | LIDIVDQLKNYVNDLVPEFLPAPEDVETNCEWSAFSCFQKA | | |
| | | QLKSANTGNNERIINESIKKLKRKPPSTNPGRRQKDRLTCP | | |
| | | SCDSYEKKPPKEFLERFKSLLQKMIHQHLSSRTHGSEDS | | |
| IL-21_5624 | V98E, R114G, | MRSSPGNMERIVICLMVIFLGTLVHKSSSQGQDRHMIRMRQ | 0.864 | 1276 |
| | R115G | LIDIVDQLKNYVNDLVPEFLPAPEDVETNCEWSAFSCFQKA | | |
| | | QLKSANTGNNERIINESIKKLKRKPPSTNAGGGQKHRLTCP | | |
| | | SCDSYEKKPPKEFLERFKSLLQKMIHQHLSSRTHGSEDS | | |
| IL-21_5246 | K81D, P107V, | MRSSPGNMERIVICLMVIFLGTLVHKSSSQGQDRHMIRMRQ | 0.865 | 1277 |
| | R114Q, T121E | LIDIVDQLKNYVNDLVPEFLPAPEDVETNCEWSAFSCFQDA | | |
| | | QLKSANTGNNERIINVSIKKLKRKVPSTNAGQRQKHRLECP | | |
| | | SCDSYEKKPPKEFLERFKSLLQKMIHQHLSSRTHGSEDS | | |
| IL-21_5012 | P107V, R114Q, | MRSSPGNMERIVICLMVIFLGTLVHKSSSQGQDRHMIRMRQ | 0.865 | 1278 |
| | R115D, H118D | LIDIVDQLKNYVNDLVPEFLPAPEDVETNCEWSAFSCFQKA | | |
| | | QLKSANTGNNERIINVSIKKLKRKVPSTNAGQDQKDRLTCP | | |
| | | SCDSYEKKPPKEFLERFKSLLQKMIHQHLSSRTHGSEDS | | |
| IL-21_5423 | N70G, V98E, | MRSSPGNMERIVICLMVIFLGTLVHKSSSQGQDRHMIRMRQ | 0.865 | 1279 |
| | R114G, R119T | LIDIVDQLKNYVNDLVPEFLPAPEDVETGCEWSAFSCFQKA | | |
| | | QLKSANTGNNERIINESIKKLKRKPPSTNAGGRQKHTLTCP | | |
| | | SCDSYEKKPPKEFLERFKSLLQKMIHQHLSSRTHGSEDS | | |
| IL-21_5672 | K81D, R114Q, | MRSSPGNMERIVICLMVIFLGTLVHKSSSQGQDRHMIRMRQ | 0.865 | 1280 |
| | R115G | LIDIVDQLKNYVNDLVPEFLPAPEDVETNCEWSAFSCFQDA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5228 | K81D, T110D, | MRSSPGNMERIVICLMVIFLGTLVHKSSSQGQDRHMIRMRQ | 0.866 | 1281 |
| | A112P, R115G | LIDIVDQLKNYVNDLVPEFLPAPEDVETNCEWSAFSCFQDA | | |
| | | QLKSANTGNNERIINVSIKKLKRKPPSDNPGRGQKHRLTCP | | |
| | | SCDSYEKKPPKEFLERFKSLLQKMIHQHLSSRTHGSEDS | | |
| IL-21_5247 | K81D, P107V, | MRSSPGNMERIVICLMVIFLGTLVHKSSSQGQDRHMIRMRQ | 0.866 | 1282 |
| | R114Q, R119T | LIDIVDQLKNYVNDLVPEFLPAPEDVETNCEWSAFSCFQDA | | |
| | | QLKSANTGNNERIINVSIKKLKRKVPSTNAGQRQKHTLTCP | | |
| | | SCDSYEKKPPKEFLERFKSLLQKMIHQHLSSRTHGSEDS | | |
| IL-21_5488 | N70G, K81D, | MRSSPGNMERIVICLMVIFLGTLVHKSSSQGQDRHMIRMRQ | 0.867 | 1283 |
| | T110D, R114Q | LIDIVDQLKNYVNDLVPEFLPAPEDVETGCEWSAFSCFQDA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5989 | H35F, V98E, | MRSSPGNMERIVICLMVIFLGTLVHKSSSQGQDREMIRMRQ | 0.868 | 1284 |
| | P107V, R115D | LIDIVDQLKNYVNDLVPEFLPAPEDVETNCEWSAFSCFQKA | | |
| | | QLKSANTGNNERIINESIKKLKRKVPSTNAGRDQKHRLTCP | | |
| | | SCDSYEKKPPKEFLERFKSLLQKMIHQHLSSRTHGSEDS | | |
| IL-21_5478 | N70G, K81D, | MRSSPGNMERIVICLMVIFLGTLVHKSSSQGQDRHMIRMRQ | 0.869 | 1285 |
| | A112P, R115G | LIDIVDQLKNYVNDLVPEFLPAPEDVETGCEWSAFSCFQDA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5235 | K81D, P107V, | MRSSPGNMERIVICLMVIFLGTLVHKSSSQGQDRHMIRMRQ | 0.87 | 1286 |
| | R115G, T121E | LIDIVDQLKNYVNDLVPEFLPAPEDVETNCEWSAFSCFQDA | | |
| | | QLKSANTGNNERIINVSIKKLKRKVPSTNAGRGQKHRLECP | | |
| | | SCDSYEKKPPKEFLERFKSLLQKMIHQHLSSRTHGSEDS | | |
| IL-21_5439 | N70G, V98E, | MRSSPGNMERIVICLMVIFLGTLVHKSSSQGQDRHMIRMRQ | 0.87 | 1287 |
| | T110D, T121E | LIDIVDQLKNYVNDLVPEFLPAPEDVETGCEWSAFSCFQKA | | |
| | | QLKSANTGNNERIINESIKKLKRKPPSDNAGRRQKHRLECP | | |
| | | SCDSYEKKPPKEFLERFKSLLQKMIHQHLSSRTHGSEDS | | |
| IL-21_5641 | V98E, T110D, | MRSSPGNMERIVICLMVIFLGTLVHKSSSQGQDRHMIRMRQ | 0.871 | 1288 |
| | R115G | LIDIVDQLKNYVNDLVPEFLPAPEDVETNCEWSAFSCFQKA | | |
| | | QLKSANTGNNERIINESIKKLKRKPPSDNAGRGQKHRLTCP | | |
| | | SCDSYEKKPPKEFLERFKSLLQKMIHQHLSSRTHGSEDS | | |
| IL-21_5465 | N70G, K81D, | MRSSPGNMERIVICLMVIFLGTLVHKSSSQGQDRHMIRMRQ | 0.871 | 1289 |
| | R114G, T121E | LIDIVDQLKNYVNDLVPEFLPAPEDVETGCEWSAFSCFQDA | | |
| | | QLKSANTGNNERIINVSIKKLKRKPPSTNAGGRQKHRLECP | | |
| | | SCDSYEKKPPKEFLERFKSLLQKMIHQHLSSRTHGSEDS | | |
| IL-21_5963 | H35F, P107V, | MRSSPGNMERIVICLMVIFLGTLVHKSSSQGQDREMIRMRQ | 0.872 | 1290 |
| | R114G, R115D | LIDIVDQLKNYVNDLVPEFLPAPEDVETNCEWSAFSCFQKA | | |
| | | QLKSANTGNNERIINVSIKKLKRKVPSTNAGGDQKHRLTCP | | |
| | | SCDSYEKKPPKEFLERFKSLLQKMIHQHLSSRTHGSEDS | | |
| IL-21_5324 | N70G, R114G, | MRSSPGNMERIVICLMVIFLGTLVHKSSSQGQDRHMIRMRQ | 0.872 | 1291 |
| | R115D, H118D | LIDIVDQLKNYVNDLVPEFLPAPEDVETGCEWSAFSCFQKA | | |
| | | QLKSANTGNNERIINVSIKKLKRKPPSTNAGGDQKDRLTCP | | |
| | | SCDSYEKKPPKEFLERFKSLLQKMIHQHLSSRTHGSEDS | | |
| IL-21_5227 | K81D, T110D, | MRSSPGNMERIVICLMVIFLGTLVHKSSSQGQDRHMIRMRQ | 0.873 | 1292 |
| | A112P, H118D | LIDIVDQLKNYVNDLVPEFLPAPEDVETNCEWSAFSCFQDA | | |
| | | QLKSANTGNNERIINVSIKKLKRKPPSDNPGRRQKDRLTCP | | |
| | | SCDSYEKKPPKEFLERFKSLLQKMIHQHLSSRTHGSEDS | | |
| IL-21_5197 | K81D, A112P, | MRSSPGNMERIVICLMVIFLGTLVHKSSSQGQDRHMIRMRQ | 0.873 | 1293 |
| | R114G, R119T | LIDIVDQLKNYVNDLVPEFLPAPEDVETNCEWSAFSCFQDA | | |
| | | QLKSANTGNNERIINVSIKKLKRKPPSTNPGGRQKHTLTCP | | |
| | | SCDSYEKKPPKEFLERFKSLLQKMIHQHLSSRTHGSEDS | | |
| IL-21_5663 | K81D, R115D, | MRSSPGNMERIVICLMVIFLGTLVHKSSSQGQDRHMIRMRQ | 0.873 | 1294 |
| | H118D | LIDIVDQLKNYVNDLVPEFLPAPEDVETNCEWSAFSCFQDA | | |
| | | QLKSANTGNNERIINVSIKKLKRKPPSTNAGRDQKDRLTCP | | |
| | | SCDSYEKKPPKEFLERFKSLLQKMIHQHLSSRTHGSEDS | | |
| IL-21_6198 | H35I, N70G, | MRSSPGNMERIVICLMVIFLGTLVHKSSSQGQDRIMIRMRQ | 0.874 | 1295 |
| | K81D, P107V | LIDIVDQLKNYVNDLVPEFLPAPEDVETGCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_6122 | H35I, V98E, | MRSSPGNMERIVICLMVIFLGTLVHKSSSQGQDRIMIRMRQ | 0.875 | 1296 |
| | R114G, R115G | LIDIVDQLKNYVNDLVPEFLPAPEDVETNCEWSAFSCFQKA | | |
| | | QLKSANTGNNERIINESIKKLKRKPPSTNAGGGQKHRLTCP | | |
| | | SCDSYEKKPPKEFLERFKSLLQKMIHQHLSSRTHGSEDS | | |
| IL-21_6357 | H35I, V98E, | MRSSPGNMERIVICLMVIFLGTLVHKSSSQGQDRIMIRMRQ | 0.875 | 1297 |
| | R115G | LIDIVDQLKNYVNDLVPEFLPAPEDVETNCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_6129 | H35I, V98E, | MRSSPGNMERIVICLMVIFLGTLVHKSSSQGQDRIMIRMRQ | 0.876 | 1298 |
| | A112P, R114Q | LIDIVDQLKNYVNDLVPEFLPAPEDVETNCEWSAFSCFQKA | | |
| | | QLKSANTGNNERIINESIKKLKRKPPSTNPGQRQKHRLTCP | | |
| | | SCDSYEKKPPKEFLERFKSLLQKMIHQHLSSRTHGSEDS | | |
| IL-21_5428 | N70G, V98E, | MRSSPGNMERIVICLMVIFLGTLVHKSSSQGQDRHMIRMRQ | 0.877 | 1299 |
| | R114Q, R119T | LIDIVDQLKNYVNDLVPEFLPAPEDVETGCEWSAFSCFQKA | | |
| | | QLKSANTGNNERIINESIKKLKRKPPSTNAGQRQKHTLTCP | | |
| | | SCDSYEKKPPKEFLERFKSLLQKMIHQHLSSRTHGSEDS | | |
| IL-21_5620 | V98E, R115D, | MRSSPGNMERIVICLMVIFLGTLVHKSSSQGQDRHMIRMRQ | 0.877 | 1300 |
| | H118D | LIDIVDQLKNYVNDLVPEFLPAPEDVETNCEWSAFSCFQKA | | |
| | | QLKSANTGNNERIINESIKKLKRKPPSTNAGRDQKDRLTCP | | |
| | | SCDSYEKKPPKEFLERFKSLLQKMIHQHLSSRTHGSEDS | | |
| IL-21_5091 | V98E, A112P, | MRSSPGNMERIVICLMVIFLGTLVHKSSSQGQDRHMIRMRQ | 0.879 | 1301 |
| | R115D, H118D | LIDIVDQLKNYVNDLVPEFLPAPEDVETNCEWSAFSCFQKA | | |
| | | QLKSANTGNNERIINESIKKLKRKPPSTNPGRDQKDRLTCP | | |
| | | SCDSYEKKPPKEFLERFKSLLQKMIHQHLSSRTHGSEDS | | |
| IL-21_5427 | N70G, V98E, | MRSSPGNMERIVICLMVIFLGTLVHKSSSQGQDRHMIRMRQ | 0.879 | 1302 |
| | R114Q, T121E | LIDIVDQLKNYVNDLVPEFLPAPEDVETGCEWSAFSCFQKA | | |
| | | QLKSANTGNNERIINESIKKLKRKPPSTNAGQRQKHRLECP | | |
| | | SCDSYEKKPPKEFLERFKSLLQKMIHQHLSSRTHGSEDS | | |
| IL-21_5160 | V98E, P107V, | MRSSPGNMERIVICLMVIFLGTLVHKSSSQGQDRHMIRMRQ | 0.879 | 1303 |
| | T110D, R114Q | LIDIVDQLKNYVNDLVPEFLPAPEDVETNCEWSAFSCFQKA | | |
| | | QLKSANTGNNERIINESIKKLKRKVPSDNAGQRQKHRLTCP | | |
| | | SCDSYEKKPPKEFLERFKSLLQKMIHQHLSSRTHGSEDS | | |
| IL-21_5116 | V98E, T110D, | MRSSPGNMERIVICLMVIFLGTLVHKSSSQGQDRHMIRMRQ | 0.879 | 1304 |
| | R114Q, T121E | LIDIVDQLKNYVNDLVPEFLPAPEDVETNCEWSAFSCFQKA | | |
| | | QLKSANTGNNERIINESIKKLKRKPPSDNAGQRQKHRLECP | | |
| | | SCDSYEKKPPKEFLERFKSLLQKMIHQHLSSRTHGSEDS | | |
| IL-21_6314 | H35F, K81D, | MRSSPGNMERIVICLMVIFLGTLVHKSSSQGQDREMIRMRQ | 0.88 | 1305 |
| | R114Q | LIDIVDQLKNYVNDLVPEFLPAPEDVETNCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5127 | V98E, T110D, | MRSSPGNMERIVICLMVIFLGTLVHKSSSQGQDRHMIRMRQ | 0.881 | 1306 |
| | A112P, R114Q | LIDIVDQLKNYVNDLVPEFLPAPEDVETNCEWSAFSCFQKA | | |
| | | QLKSANTGNNERIINESIKKLKRKPPSDNPGQRQKHRLTCP | | |
| | | SCDSYEKKPPKEFLERFKSLLQKMIHQHLSSRTHGSEDS | | |
| IL-21_5084 | V98E, A112P, | MRSSPGNMERIVICLMVIFLGTLVHKSSSQGQDRHMIRMRQ | 0.881 | 1307 |
| | H118D, T121E | LIDIVDQLKNYVNDLVPEFLPAPEDVETNCEWSAFSCFQKA | | |
| | | QLKSANTGNNERIINESIKKLKRKPPSTNPGRRQKDRLECP | | |
| | | SCDSYEKKPPKEFLERFKSLLQKMIHQHLSSRTHGSEDS | | |
| IL-21_6147 | H35I, K81D, | MRSSPGNMERIVICLMVIFLGTLVHKSSSQGQDRIMIRMRQ | 0.882 | 1308 |
| | A112P, R114G | LIDIVDQLKNYVNDLVPEFLPAPEDVETNCEWSAFSCFQDA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5490 | N70G, K81D, | MRSSPGNMERIVICLMVIFLGTLVHKSSSQGQDRHMIRMRQ | 0.882 | 1309 |
| | P107V, T121E | LIDIVDQLKNYVNDLVPEFLPAPEDVETGCEWSAFSCFQDA | | |
| | | QLKSANTGNNERIINVSIKKLKRKVPSTNAGRRQKHRLECP | | |
| | | SCDSYEKKPPKEFLERFKSLLQKMIHQHLSSRTHGSEDS | | |
| IL-21_6150 | H35I, K81D, | MRSSPGNMERIVICLMVIFLGTLVHKSSSQGQDRIMIRMRQ | 0.882 | 1310 |
| | T110D, R115D | LIDIVDQLKNYVNDLVPEFLPAPEDVETNCEWSAFSCFQDA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_5206 | K81D, T110D, | MRSSPGNMERIVICLMVIFLGTLVHKSSSQGQDRHMIRMRQ | 0.884 | 1311 |
| | R119T, T121E | LIDIVDQLKNYVNDLVPEFLPAPEDVETNCEWSAFSCFQDA | | |
| | | QLKSANTGNNERIINVSIKKLKRKPPSDNAGRRQKHTLECP | | |
| | | SCDSYEKKPPKEFLERFKSLLQKMIHQHLSSRTHGSEDS | | |
| IL-21_5629 | V98E, R114Q, | MRSSPGNMERIVICLMVIFLGTLVHKSSSQGQDRHMIRMRQ | 0.884 | 1312 |
| | R115G | LIDIVDQLKNYVNDLVPEFLPAPEDVETNCEWSAFSCFQKA | | |
| | | QLKSANTGNNERIINESIKKLKRKPPSTNAGQGQKHRLTCP | | |
| | | SCDSYEKKPPKEFLERFKSLLQKMIHQHLSSRTHGSEDS | | |
| IL-21_5165 | K81D, R115G, | MRSSPGNMERIVICLMVIFLGTLVHKSSSQGQDRHMIRMRQ | 0.884 | 1313 |
| | H118D, R119T | LIDIVDQLKNYVNDLVPEFLPAPEDVETNCEWSAFSCFQDA | | |
| | | QLKSANTGNNERIINVSIKKLKRKPPSTNAGRGQKDTLTCP | | |
| | | SCDSYEKKPPKEFLERFKSLLQKMIHQHLSSRTHGSEDS | | |
| IL-21_5971 | H35F, P107V, | MRSSPGNMERIVICLMVIFLGTLVHKSSSQGQDRFMIRMRQ | 0.885 | 1314 |
| | T110D, R115D | LIDIVDQLKNYVNDLVPEFLPAPEDVETNCEWSAFSCFQKA | | |
| | | QLKSANTGNNERIINVSIKKLKRKVPSDNAGRDQKHRLTCP | | |
| | | SCDSYEKKPPKEFLERFKSLLQKMIHQHLSSRTHGSEDS | | |
| IL-21_6049 | H35F, N70G, | MRSSPGNMERIVICLMVIFLGTLVHKSSSQGQDREMIRMRQ | 0.888 | 1315 |
| | K81D, R114Q | LIDIVDQLKNYVNDLVPEFLPAPEDVETGCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_6044 | H35F, N70G, | MRSSPGNMERIVICLMVIFLGTLVHKSSSQGQDREMIRMRQ | 0.888 | 1316 |
| | V98E, T110D | LIDIVDQLKNYVNDLVPEFLPAPEDVETGCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5900 | I45T, K81D, | MRSSPGNMERIVICLMVIFLGTLVHKSSSQGQDRHMIRMRQ | 0.888 | 1317 |
| | T110D, R114G | LIDTVDQLKNYVNDLVPEFLPAPEDVETNCEWSAFSCFQDA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5392 | N70G, P107V, | MRSSPGNMERIVICLMVIFLGTLVHKSSSQGQDRHMIRMRQ | 0.888 | 1318 |
| | R114G, R115D | LIDIVDQLKNYVNDLVPEFLPAPEDVETGCEWSAFSCFQKA | | |
| | | QLKSANTGNNERIINVSIKKLKRKVPSTNAGGDQKHRLTCP | | |
| | | SCDSYEKKPPKEFLERFKSLLQKMIHQHLSSRTHGSEDS | | |
| IL-21_6146 | H35I, K81D, | MRSSPGNMERIVICLMVIFLGTLVHKSSSQGQDRIMIRMRQ | 0.89 | 1319 |
| | A112P, R115D | LIDIVDQLKNYVNDLVPEFLPAPEDVETNCEWSAFSCFQDA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5154 | V98E, P107V, | MRSSPGNMERIVICLMVIFLGTLVHKSSSQGQDRHMIRMRQ | 0.89 | 1320 |
| | T110D, T121E | LIDIVDQLKNYVNDLVPEFLPAPEDVETNCEWSAFSCFQKA | | |
| | | QLKSANTGNNERIINESIKKLKRKVPSDNAGRRQKHRLECP | | |
| | | SCDSYEKKPPKEFLERFKSLLQKMIHQHLSSRTHGSEDS | | |
| IL-21_5767 | N70G, K81D, | MRSSPGNMERIVICLMVIFLGTLVHKSSSQGQDRHMIRMRQ | 0.89 | 1321 |
| | R114Q | LIDIVDQLKNYVNDLVPEFLPAPEDVETGCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5191 | K81D, A112P, | MRSSPGNMERIVICLMVIFLGTLVHKSSSQGQDRHMIRMRQ | 0.89 | 1322 |
| | R115G, R119T | LIDIVDQLKNYVNDLVPEFLPAPEDVETNCEWSAFSCFQDA | | |
| | | QLKSANTGNNERIINVSIKKLKRKPPSTNPGRGQKHTLTCP | | |
| | | SCDSYEKKPPKEFLERFKSLLQKMIHQHLSSRTHGSEDS | | |
| IL-21_5059 | V98E, R115G, | MRSSPGNMERIVICLMVIFLGTLVHKSSSQGQDRHMIRMRQ | 0.893 | 1323 |
| | R119T, T121E | LIDIVDQLKNYVNDLVPEFLPAPEDVETNCEWSAFSCFQKA | | |
| | | QLKSANTGNNERIINESIKKLKRKPPSTNAGRGQKHTLECP | | |
| | | SCDSYEKKPPKEFLERFKSLLQKMIHQHLSSRTHGSEDS | | |
| IL-21_5193 | K81D, A112P, | MRSSPGNMERIVICLMVIFLGTLVHKSSSQGQDRHMIRMRQ | 0.894 | 1324 |
| | R115D, T121E | LIDIVDQLKNYVNDLVPEFLPAPEDVETNCEWSAFSCFQDA | | |
| | | QLKSANTGNNERIINVSIKKLKRKPPSTNPGRDQKHRLECP | | |
| | | SCDSYEKKPPKEFLERFKSLLQKMIHQHLSSRTHGSEDS | | |
| IL-21_5416 | N70G, V98E, | MRSSPGNMERIVICLMVIFLGTLVHKSSSQGQDRHMIRMRQ | 0.894 | 1325 |
| | R115G, T121E | LIDIVDQLKNYVNDLVPEFLPAPEDVETGCEWSAFSCFQKA | | |
| | | QLKSANTGNNERIINESIKKLKRKPPSTNAGRGQKHRLECP | | |
| | | SCDSYEKKPPKEFLERFKSLLQKMIHQHLSSRTHGSEDS | | |
| IL-21_6367 | H35I, K81D, | MRSSPGNMERIVICLMVIFLGTLVHKSSSQGQDRIMIRMRQ | 0.894 | 1326 |
| | R114Q | LIDIVDQLKNYVNDLVPEFLPAPEDVETNCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5659 | K81D, R115G, | MRSSPGNMERIVICLMVIFLGTLVHKSSSQGQDRHMIRMRQ | 0.895 | 1327 |
| | R119T | LIDIVDQLKNYVNDLVPEFLPAPEDVETNCEWSAFSCFQDA | | |
| | | QLKSANTGNNERIINVSIKKLKRKPPSTNAGRGQKHTLTCP | | |
| | | SCDSYEKKPPKEFLERFKSLLQKMIHQHLSSRTHGSEDS | | |
| IL-21_5914 | I45T, K81D, | MRSSPGNMERIVICLMVIFLGTLVHKSSSQGQDRHMIRMRQ | 0.896 | 1328 |
| | V98E, T110D | LIDTVDQLKNYVNDLVPEFLPAPEDVETNCEWSAFSCFQDA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5453 | N70G, V98E, | MRSSPGNMERIVICLMVIFLGTLVHKSSSQGQDRHMIRMRQ | 0.896 | 1329 |
| | P107V, R114Q | LIDIVDQLKNYVNDLVPEFLPAPEDVETGCEWSAFSCFQKA | | |
| | | QLKSANTGNNERIINESIKKLKRKVPSTNAGQRQKHRLTCP | | |
| | | SCDSYEKKPPKEFLERFKSLLQKMIHQHLSSRTHGSEDS | | |
| IL-21_5095 | V98E, A112P, | MRSSPGNMERIVICLMVIFLGTLVHKSSSQGQDRHMIRMRQ | 0.897 | 1330 |
| | R114G, R115G | LIDIVDQLKNYVNDLVPEFLPAPEDVETNCEWSAFSCFQKA | | |
| | | QLKSANTGNNERIINESIKKLKRKPPSTNPGGGQKHRLTCP | | |
| | | SCDSYEKKPPKEFLERFKSLLQKMIHQHLSSRTHGSEDS | | |
| IL-21_5190 | K81D, A112P, | MRSSPGNMERIVICLMVIFLGTLVHKSSSQGQDRHMIRMRQ | 0.898 | 1331 |
| | R115G, T121E | LIDIVDQLKNYVNDLVPEFLPAPEDVETNCEWSAFSCFQDA | | |
| | | QLKSANTGNNERIINVSIKKLKRKPPSTNPGRGQKHRLECP | | |
| | | SCDSYEKKPPKEFLERFKSLLQKMIHQHLSSRTHGSEDS | | |
| IL-21_5071 | V98E, R114G, | MRSSPGNMERIVICLMVIFLGTLVHKSSSQGQDRHMIRMRQ | 0.901 | 1332 |
| | R115D, T121E | LIDIVDQLKNYVNDLVPEFLPAPEDVETNCEWSAFSCFQKA | | |
| | | QLKSANTGNNERIINESIKKLKRKPPSTNAGGDQKHRLECP | | |
| | | SCDSYEKKPPKEFLERFKSLLQKMIHQHLSSRTHGSEDS | | |
| IL-21_5254 | K81D, P107V, | MRSSPGNMERIVICLMVIFLGTLVHKSSSQGQDRHMIRMRQ | 0.901 | 1333 |
| | A112P, R115G | LIDIVDQLKNYVNDLVPEFLPAPEDVETNCEWSAFSCFQDA | | |
| | | QLKSANTGNNERIINVSIKKLKRKVPSTNPGRGQKHRLTCP | | |
| | | SCDSYEKKPPKEFLERFKSLLQKMIHQHLSSRTHGSEDS | | |
| IL-21_5231 | K81D, T110D, | MRSSPGNMERIVICLMVIFLGTLVHKSSSQGQDRHMIRMRQ | 0.901 | 1334 |
| | A112P, R114Q | LIDIVDQLKNYVNDLVPEFLPAPEDVETNCEWSAFSCFQDA | | |
| | | QLKSANTGNNERIINVSIKKLKRKPPSDNPGQRQKHRLTCP | | |
| | | SCDSYEKKPPKEFLERFKSLLQKMIHQHLSSRTHGSEDS | | |
| IL-21_5953 | H35F, A112P, | MRSSPGNMERIVICLMVIFLGTLVHKSSSQGQDREMIRMRQ | 0.901 | 1335 |
| | R114Q, R115D | LIDIVDQLKNYVNDLVPEFLPAPEDVETNCEWSAFSCFQKA | | |
| | | QLKSANTGNNERIINVSIKKLKRKPPSTNPGQDQKHRLTCP | | |
| | | SCDSYEKKPPKEFLERFKSLLQKMIHQHLSSRTHGSEDS | | |
| IL-21_5697 | K81D, P107V, | MRSSPGNMERIVICLMVIFLGTLVHKSSSQGQDRHMIRMRQ | 0.901 | 1336 |
| | T110D | LIDIVDQLKNYVNDLVPEFLPAPEDVETNCEWSAFSCFQDA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5241 | K81D, P107V, | MRSSPGNMERIVICLMVIFLGTLVHKSSSQGQDRHMIRMRQ | 0.902 | 1337 |
| | R114G, T121E | LIDIVDQLKNYVNDLVPEFLPAPEDVETNCEWSAFSCFQDA | | |
| | | QLKSANTGNNERIINVSIKKLKRKVPSTNAGGRQKHRLECP | | |
| | | SCDSYEKKPPKEFLERFKSLLQKMIHQHLSSRTHGSEDS | | |
| IL-21_6226 | H35I, I45T, | MRSSPGNMERIVICLMVIFLGTLVHKSSSQGQDRIMIRMRQ | 0.902 | 1338 |
| | K81D, R115G | LIDTVDQLKNYVNDLVPEFLPAPEDVETNCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_6143 | H35I, K81D, | MRSSPGNMERIVICLMVIFLGTLVHKSSSQGQDRIMIRMRQ | 0.903 | 1339 |
| | R114Q, R115G | LIDIVDQLKNYVNDLVPEFLPAPEDVETNCEWSAFSCFQDA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5131 | V98E, P107V, | MRSSPGNMERIVICLMVIFLGTLVHKSSSQGQDRHMIRMRQ | 0.903 | 1340 |
| | R115G, T121E | LIDIVDQLKNYVNDLVPEFLPAPEDVETNCEWSAFSCFQKA | | |
| | | QLKSANTGNNERIINESIKKLKRKVPSTNAGRGQKHRLECP | | |
| | | SCDSYEKKPPKEFLERFKSLLQKMIHQHLSSRTHGSEDS | | |
| IL-21_5199 | K81D, A112P, | MRSSPGNMERIVICLMVIFLGTLVHKSSSQGQDRHMIRMRQ | 0.904 | 1341 |
| | R114G, R115G | LIDIVDQLKNYVNDLVPEFLPAPEDVETNCEWSAFSCFQDA | | |
| | | QLKSANTGNNERIINVSIKKLKRKPPSTNPGGGQKHRLTCP | | |
| | | SCDSYEKKPPKEFLERFKSLLQKMIHQHLSSRTHGSEDS | | |
| IL-21_5230 | K81D, T110D, | MRSSPGNMERIVICLMVIFLGTLVHKSSSQGQDRHMIRMRQ | 0.904 | 1342 |
| | A112P, R114G | LIDIVDQLKNYVNDLVPEFLPAPEDVETNCEWSAFSCFQDA | | |
| | | QLKSANTGNNERIINVSIKKLKRKPPSDNPGGRQKHRLTCP | | |
| | | SCDSYEKKPPKEFLERFKSLLQKMIHQHLSSRTHGSEDS | | |
| IL-21_6195 | H35I, N70G, | MRSSPGNMERIVICLMVIFLGTLVHKSSSQGQDRIMIRMRQ | 0.904 | 1343 |
| | K81D, R114Q | LIDIVDQLKNYVNDLVPEFLPAPEDVETGCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5075 | V98E, R114Q, | MRSSPGNMERIVICLMVIFLGTLVHKSSSQGQDRHMIRMRQ | 0.904 | 1344 |
| | H118D, T121E | LIDIVDQLKNYVNDLVPEFLPAPEDVETNCEWSAFSCFQKA | | |
| | | QLKSANTGNNERIINESIKKLKRKPPSTNAGQRQKDRLECP | | |
| | | SCDSYEKKPPKEFLERFKSLLQKMIHQHLSSRTHGSEDS | | |
| IL-21_5954 | H35F, T110D, | MRSSPGNMERIVICLMVIFLGTLVHKSSSQGQDREMIRMRQ | 0.905 | 1345 |
| | R114G, R115G | LIDIVDQLKNYVNDLVPEFLPAPEDVETNCEWSAFSCFQKA | | |
| | | QLKSANTGNNERIINVSIKKLKRKPPSDNAGGGQKHRLTCP | | |
| | | SCDSYEKKPPKEFLERFKSLLQKMIHQHLSSRTHGSEDS | | |
| IL-21_5333 | N70G, R114Q, | MRSSPGNMERIVICLMVIFLGTLVHKSSSQGQDRHMIRMRQ | 0.907 | 1346 |
| | R115D, H118D | LIDIVDQLKNYVNDLVPEFLPAPEDVETGCEWSAFSCFQKA | | |
| | | QLKSANTGNNERIINVSIKKLKRKPPSTNAGQDQKDRLTCP | | |
| | | SCDSYEKKPPKEFLERFKSLLQKMIHQHLSSRTHGSEDS | | |
| IL-21_5657 | K81D, H118D, | MRSSPGNMERIVICLMVIFLGTLVHKSSSQGQDRHMIRMRQ | 0.907 | 1347 |
| | R119T | LIDIVDQLKNYVNDLVPEFLPAPEDVETNCEWSAFSCFQDA | | |
| | | QLKSANTGNNERIINVSIKKLKRKPPSTNAGRRQKDTLTCP | | |
| | | SCDSYEKKPPKEFLERFKSLLQKMIHQHLSSRTHGSEDS | | |
| IL-21_6359 | H35I, V98E, | MRSSPGNMERIVICLMVIFLGTLVHKSSSQGQDRIMIRMRQ | 0.908 | 1348 |
| | R114G | LIDIVDQLKNYVNDLVPEFLPAPEDVETNCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5045 | P107V, T110D, | MRSSPGNMERIVICLMVIFLGTLVHKSSSQGQDRHMIRMRQ | 0.908 | 1349 |
| | R114G, R115D | LIDIVDQLKNYVNDLVPEFLPAPEDVETNCEWSAFSCFQKA | | |
| | | QLKSANTGNNERIINVSIKKLKRKVPSDNAGGDQKHRLTCP | | |
| | | SCDSYEKKPPKEFLERFKSLLQKMIHQHLSSRTHGSEDS | | |
| IL-21_5649 | V98E, P107V, | MRSSPGNMERIVICLMVIFLGTLVHKSSSQGQDRHMIRMRQ | 0.91 | 1350 |
| | R115G | LIDIVDQLKNYVNDLVPEFLPAPEDVETNCEWSAFSCFQKA | | |
| | | QLKSANTGNNERIINESIKKLKRKVPSTNAGRGQKHRLTCP | | |
| | | SCDSYEKKPPKEFLERFKSLLQKMIHQHLSSRTHGSEDS | | |
| IL-21_6306 | H35F, V98E, | MRSSPGNMERIVICLMVIFLGTLVHKSSSQGQDREMIRMRQ | 0.91 | 1351 |
| | R114G | LIDIVDQLKNYVNDLVPEFLPAPEDVETNCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5481 | N70G, K81D, | MRSSPGNMERIVICLMVIFLGTLVHKSSSQGQDRHMIRMRQ | 0.912 | 1352 |
| | A112P, R114Q | LIDIVDQLKNYVNDLVPEFLPAPEDVETGCEWSAFSCFQDA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5981 | H35F, V98E, | MRSSPGNMERIVICLMVIFLGTLVHKSSSQGQDREMIRMRQ | 0.912 | 1353 |
| | A112P, R114G | LIDIVDQLKNYVNDLVPEFLPAPEDVETNCEWSAFSCFQKA | | |
| | | QLKSANTGNNERIINESIKKLKRKPPSTNPGGRQKHRLTCP | | |
| | | SCDSYEKKPPKEFLERFKSLLQKMIHQHLSSRTHGSEDS | | |
| IL-21_5449 | N70G, V98E, | MRSSPGNMERIVICLMVIFLGTLVHKSSSQGQDRHMIRMRQ | 0.913 | 1354 |
| | P107V, H118D | LIDIVDQLKNYVNDLVPEFLPAPEDVETGCEWSAFSCFQKA | | |
| | | QLKSANTGNNERIINESIKKLKRKVPSTNAGRRQKDRLTCP | | |
| | | SCDSYEKKPPKEFLERFKSLLQKMIHQHLSSRTHGSEDS | | |
| IL-21_5936 | I45T, N70G, | MRSSPGNMERIVICLMVIFLGTLVHKSSSQGQDRHMIRMRQ | 0.914 | 1355 |
| | V98E, R115D | LIDTVDQLKNYVNDLVPEFLPAPEDVETGCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5656 | K81D, H118D, | MRSSPGNMERIVICLMVIFLGTLVHKSSSQGQDRHMIRMRQ | 0.914 | 1356 |
| | T121E | LIDIVDQLKNYVNDLVPEFLPAPEDVETNCEWSAFSCFQDA | | |
| | | QLKSANTGNNERIINVSIKKLKRKPPSTNAGRRQKDRLECP | | |
| | | SCDSYEKKPPKEFLERFKSLLQKMIHQHLSSRTHGSEDS | | |
| IL-21_4949 | T110D, R115D, | MRSSPGNMERIVICLMVIFLGTLVHKSSSQGQDRHMIRMRQ | 0.915 | 1357 |
| | H118D, T121E | LIDIVDQLKNYVNDLVPEFLPAPEDVETNCEWSAFSCFQKA | | |
| | | QLKSANTGNNERIINVSIKKLKRKPPSDNAGRDQKDRLECP | | |
| | | SCDSYEKKPPKEFLERFKSLLQKMIHQHLSSRTHGSEDS | | |
| IL-21_5694 | K81D, P107V, | MRSSPGNMERIVICLMVIFLGTLVHKSSSQGQDRHMIRMRQ | 0.916 | 1358 |
| | R114G | LIDIVDQLKNYVNDLVPEFLPAPEDVETNCEWSAFSCFQDA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5482 | N70G, K81D, | MRSSPGNMERIVICLMVIFLGTLVHKSSSQGQDRHMIRMRQ | 0.916 | 1359 |
| | T110D, T121E | LIDIVDQLKNYVNDLVPEFLPAPEDVETGCEWSAFSCFQDA | | |
| | | QLKSANTGNNERIINVSIKKLKRKPPSDNAGRRQKHRLECP | | |
| | | SCDSYEKKPPKEFLERFKSLLQKMIHQHLSSRTHGSEDS | | |
| IL-21_5437 | N70G, V98E, | MRSSPGNMERIVICLMVIFLGTLVHKSSSQGQDRHMIRMRQ | 0.916 | 1360 |
| | A112P, R114G | LIDIVDQLKNYVNDLVPEFLPAPEDVETGCEWSAFSCFQKA | | |
| | | QLKSANTGNNERIINESIKKLKRKPPSTNPGGRQKHRLTCP | | |
| | | SCDSYEKKPPKEFLERFKSLLQKMIHQHLSSRTHGSEDS | | |
| IL-21_4993 | P107V, R115D, | MRSSPGNMERIVICLMVIFLGTLVHKSSSQGQDRHMIRMRQ | 0.917 | 1361 |
| | H118D, T121E | LIDIVDQLKNYVNDLVPEFLPAPEDVETNCEWSAFSCFQKA | | |
| | | QLKSANTGNNERIINVSIKKLKRKVPSTNAGRDQKDRLECP | | |
| | | SCDSYEKKPPKEFLERFKSLLQKMIHQHLSSRTHGSEDS | | |
| IL-21_5669 | K81D, R114Q, | MRSSPGNMERIVICLMVIFLGTLVHKSSSQGQDRHMIRMRQ | 0.917 | 1362 |
| | T121E | LIDIVDQLKNYVNDLVPEFLPAPEDVETNCEWSAFSCFQDA | | |
| | | QLKSANTGNNERIINVSIKKLKRKPPSTNAGQRQKHRLECP | | |
| | | SCDSYEKKPPKEFLERFKSLLQKMIHQHLSSRTHGSEDS | | |
| IL-21_5678 | K81D, A112P, | MRSSPGNMERIVICLMVIFLGTLVHKSSSQGQDRHMIRMRQ | 0.918 | 1363 |
| | R115D | LIDIVDQLKNYVNDLVPEFLPAPEDVETNCEWSAFSCFQDA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_6140 | H35I, V98E, | MRSSPGNMERIVICLMVIFLGTLVHKSSSQGQDRIMIRMRQ | 0.918 | 1364 |
| | P107V, T110D | LIDIVDQLKNYVNDLVPEFLPAPEDVETNCEWSAFSCFQKA | | |
| | | QLKSANTGNNERIINESIKKLKRKVPSDNAGRRQKHRLTCP | | |
| | | SCDSYEKKPPKEFLERFKSLLQKMIHQHLSSRTHGSEDS | | |
| IL-21_5087 | V98E, A112P, | MRSSPGNMERIVICLMVIFLGTLVHKSSSQGQDRHMIRMRQ | 0.919 | 1365 |
| | R115G, R119T | LIDIVDQLKNYVNDLVPEFLPAPEDVETNCEWSAFSCFQKA | | |
| | | QLKSANTGNNERIINESIKKLKRKPPSTNPGRGQKHTLTCP | | |
| | | SCDSYEKKPPKEFLERFKSLLQKMIHQHLSSRTHGSEDS | | |
| IL-21_5985 | H35F, V98E, | MRSSPGNMERIVICLMVIFLGTLVHKSSSQGQDRFMIRMRQ | 0.919 | 1366 |
| | T110D, R114G | LIDIVDQLKNYVNDLVPEFLPAPEDVETNCEWSAFSCFQKA | | |
| | | QLKSANTGNNERIINESIKKLKRKPPSDNAGGRQKHRLTCP | | |
| | | SCDSYEKKPPKEFLERFKSLLQKMIHQHLSSRTHGSEDS | | |
| IL-21_5452 | N70G, V98E, | MRSSPGNMERIVICLMVIFLGTLVHKSSSQGQDRHMIRMRQ | 0.919 | 1367 |
| | P107V, R114G | LIDIVDQLKNYVNDLVPEFLPAPEDVETGCEWSAFSCFQKA | | |
| | | QLKSANTGNNERIINESIKKLKRKVPSTNAGGRQKHRLTCP | | |
| | | SCDSYEKKPPKEFLERFKSLLQKMIHQHLSSRTHGSEDS | | |
| IL-21_5438 | N70G, V98E, | MRSSPGNMERIVICLMVIFLGTLVHKSSSQGQDRHMIRMRQ | 0.919 | 1368 |
| | A112P, R114Q | LIDIVDQLKNYVNDLVPEFLPAPEDVETGCEWSAFSCFQKA | | |
| | | QLKSANTGNNERIINESIKKLKRKPPSTNPGQRQKHRLTCP | | |
| | | SCDSYEKKPPKEFLERFKSLLQKMIHQHLSSRTHGSEDS | | |
| IL-21_5991 | H35F, V98E, | MRSSPGNMERIVICLMVIFLGTLVHKSSSQGQDREMIRMRQ | 0.92 | 1369 |
| | P107V, R114Q | LIDIVDQLKNYVNDLVPEFLPAPEDVETNCEWSAFSCFQKA | | |
| | | QLKSANTGNNERIINESIKKLKRKVPSTNAGQRQKHRLTCP | | |
| | | SCDSYEKKPPKEFLERFKSLLQKMIHQHLSSRTHGSEDS | | |
| IL-21_5915 | I45T, K81D, | MRSSPGNMERIVICLMVIFLGTLVHKSSSQGQDRHMIRMRQ | 0.92 | 1370 |
| | V98E, P107V | LIDTVDQLKNYVNDLVPEFLPAPEDVETNCEWSAFSCFQDA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5128 | V98E, P107V, | MRSSPGNMERIVICLMVIFLGTLVHKSSSQGQDRHMIRMRQ | 0.921 | 1371 |
| | R119T, T121E | LIDIVDQLKNYVNDLVPEFLPAPEDVETNCEWSAFSCFQKA | | |
| | | QLKSANTGNNERIINESIKKLKRKVPSTNAGRRQKHTLECP | | |
| | | SCDSYEKKPPKEFLERFKSLLQKMIHQHLSSRTHGSEDS | | |
| IL-21_4994 | P107V, R115D, | MRSSPGNMERIVICLMVIFLGTLVHKSSSQGQDRHMIRMRQ | 0.921 | 1372 |
| | H118D, R119T | LIDIVDQLKNYVNDLVPEFLPAPEDVETNCEWSAFSCFQKA | | |
| | | QLKSANTGNNERIINVSIKKLKRKVPSTNAGRDQKDTLTCP | | |
| | | SCDSYEKKPPKEFLERFKSLLQKMIHQHLSSRTHGSEDS | | |
| IL-21_5459 | N70G, K81D, | MRSSPGNMERIVICLMVIFLGTLVHKSSSQGQDRHMIRMRQ | 0.921 | 1373 |
| | R115G, T121E | LIDIVDQLKNYVNDLVPEFLPAPEDVETGCEWSAFSCFQDA | | |
| | | QLKSANTGNNERIINVSIKKLKRKPPSTNAGRGQKHRLECP | | |
| | | SCDSYEKKPPKEFLERFKSLLQKMIHQHLSSRTHGSEDS | | |
| IL-21_5315 | N70G, R115D, | MRSSPGNMERIVICLMVIFLGTLVHKSSSQGQDRHMIRMRQ | 0.921 | 1374 |
| | H118D, R119T | LIDIVDQLKNYVNDLVPEFLPAPEDVETGCEWSAFSCFQKA | | |
| | | QLKSANTGNNERIINVSIKKLKRKPPSTNAGRDQKDTLTCP | | |
| | | SCDSYEKKPPKEFLERFKSLLQKMIHQHLSSRTHGSEDS | | |
| IL-21_6307 | H35F, V98E, | MRSSPGNMERIVICLMVIFLGTLVHKSSSQGQDRFMIRMRQ | 0.922 | 1375 |
| | R114Q | LIDIVDQLKNYVNDLVPEFLPAPEDVETNCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_6153 | H35I, K81D, | MRSSPGNMERIVICLMVIFLGTLVHKSSSQGQDRIMIRMRQ | 0.922 | 1376 |
| | T110D, A112P | LIDIVDQLKNYVNDLVPEFLPAPEDVETNCEWSAFSCFQDA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5759 | N70G, V98E, | MRSSPGNMERIVICLMVIFLGTLVHKSSSQGQDRHMIRMRQ | 0.922 | 1377 |
| | T110D | LIDIVDQLKNYVNDLVPEFLPAPEDVETGCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5665 | K81D, R114G, | MRSSPGNMERIVICLMVIFLGTLVHKSSSQGQDRHMIRMRQ | 0.924 | 1378 |
| | R119T | LIDIVDQLKNYVNDLVPEFLPAPEDVETNCEWSAFSCFQDA | | |
| | | QLKSANTGNNERIINVSIKKLKRKPPSTNAGGRQKHTLTCP | | |
| | | SCDSYEKKPPKEFLERFKSLLQKMIHQHLSSRTHGSEDS | | |
| IL-21_5491 | N70G, K81D, | MRSSPGNMERIVICLMVIFLGTLVHKSSSQGQDRHMIRMRQ | 0.924 | 1379 |
| | P107V, R119T | LIDIVDQLKNYVNDLVPEFLPAPEDVETGCEWSAFSCFQDA | | |
| | | QLKSANTGNNERIINVSIKKLKRKVPSTNAGRRQKHTLTCP | | |
| | | SCDSYEKKPPKEFLERFKSLLQKMIHQHLSSRTHGSEDS | | |
| IL-21_6104 | H35I, T110D, | MRSSPGNMERIVICLMVIFLGTLVHKSSSQGQDRIMIRMRQ | 0.925 | 1380 |
| | R114Q, R115D | LIDIVDQLKNYVNDLVPEFLPAPEDVETNCEWSAFSCFQKA | | |
| | | QLKSANTGNNERIINVSIKKLKRKPPSDNAGQDQKHRLTCP | | |
| | | SCDSYEKKPPKEFLERFKSLLQKMIHQHLSSRTHGSEDS | | |
| IL-21_5164 | K81D, R115G, | MRSSPGNMERIVICLMVIFLGTLVHKSSSQGQDRHMIRMRQ | 0.926 | 1381 |
| | H118D, T121E | LIDIVDQLKNYVNDLVPEFLPAPEDVETNCEWSAFSCFQDA | | |
| | | QLKSANTGNNERIINVSIKKLKRKPPSTNAGRGQKDRLECP | | |
| | | SCDSYEKKPPKEFLERFKSLLQKMIHQHLSSRTHGSEDS | | |
| IL-21_5871 | I45T, V98E, | MRSSPGNMERIVICLMVIFLGTLVHKSSSQGQDRHMIRMRQ | 0.926 | 1382 |
| | R114G, R115G | LIDTVDQLKNYVNDLVPEFLPAPEDVETNCEWSAFSCFQKA | | |
| | | QLKSANTGNNERIINESIKKLKRKPPSTNAGGGQKHRLTCP | | |
| | | SCDSYEKKPPKEFLERFKSLLQKMIHQHLSSRTHGSEDS | | |
| IL-21_5909 | I45T, K81D, | MRSSPGNMERIVICLMVIFLGTLVHKSSSQGQDRHMIRMRQ | 0.927 | 1383 |
| | V98E, R115G | LIDTVDQLKNYVNDLVPEFLPAPEDVETNCEWSAFSCFQDA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_6327 | H35F, N70G, | MRSSPGNMERIVICLMVIFLGTLVHKSSSQGQDRFMIRMRQ | 0.927 | 1384 |
| | K81D | LIDIVDQLKNYVNDLVPEFLPAPEDVETGCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5988 | H35F, V98E, | MRSSPGNMERIVICLMVIFLGTLVHKSSSQGQDRFMIRMRQ | 0.929 | 1385 |
| | P107V, R115G | LIDIVDQLKNYVNDLVPEFLPAPEDVETNCEWSAFSCFQKA | | |
| | | QLKSANTGNNERIINESIKKLKRKVPSTNAGRGQKHRLTCP | | |
| | | SCDSYEKKPPKEFLERFKSLLQKMIHQHLSSRTHGSEDS | | |
| IL-21_5652 | V98E, P107V, | MRSSPGNMERIVICLMVIFLGTLVHKSSSQGQDRHMIRMRQ | 0.929 | 1386 |
| | R114Q | LIDIVDQLKNYVNDLVPEFLPAPEDVETNCEWSAFSCFQKA | | |
| | | QLKSANTGNNERIINESIKKLKRKVPSTNAGQRQKHRLTCP | | |
| | | SCDSYEKKPPKEFLERFKSLLQKMIHQHLSSRTHGSEDS | | |
| IL-21_4968 | T110D, R114Q, | MRSSPGNMERIVICLMVIFLGTLVHKSSSQGQDRHMIRMRQ | 0.929 | 1387 |
| | R115D, H118D | LIDIVDQLKNYVNDLVPEFLPAPEDVETNCEWSAFSCFQKA | | |
| | | QLKSANTGNNERIINVSIKKLKRKPPSDNAGQDQKDRLTCP | | |
| | | SCDSYEKKPPKEFLERFKSLLQKMIHQHLSSRTHGSEDS | | |
| IL-21_5614 | V98E, H118D, | MRSSPGNMERIVICLMVIFLGTLVHKSSSQGQDRHMIRMRQ | 0.93 | 1388 |
| | R119T | LIDIVDQLKNYVNDLVPEFLPAPEDVETNCEWSAFSCFQKA | | |
| | | QLKSANTGNNERIINESIKKLKRKPPSTNAGRRQKDTLTCP | | |
| | | SCDSYEKKPPKEFLERFKSLLQKMIHQHLSSRTHGSEDS | | |
| IL-21_5648 | V98E, P107V, | MRSSPGNMERIVICLMVIFLGTLVHKSSSQGQDRHMIRMRQ | 0.93 | 1389 |
| | H118D | LIDIVDQLKNYVNDLVPEFLPAPEDVETNCEWSAFSCFQKA | | |
| | | QLKSANTGNNERIINESIKKLKRKVPSTNAGRRQKDRLTCP | | |
| | | SCDSYEKKPPKEFLERFKSLLQKMIHQHLSSRTHGSEDS | | |
| IL-21_5769 | N70G, K81D, | MRSSPGNMERIVICLMVIFLGTLVHKSSSQGQDRHMIRMRQ | 0.931 | 1390 |
| | T110D | LIDIVDQLKNYVNDLVPEFLPAPEDVETGCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5422 | N70G, V98E, | MRSSPGNMERIVICLMVIFLGTLVHKSSSQGQDRHMIRMRQ | 0.931 | 1391 |
| | R114G, T121E | LIDIVDQLKNYVNDLVPEFLPAPEDVETGCEWSAFSCFQKA | | |
| | | QLKSANTGNNERIINESIKKLKRKPPSTNAGGRQKHRLECP | | |
| | | SCDSYEKKPPKEFLERFKSLLQKMIHQHLSSRTHGSEDS | | |
| IL-21_5763 | N70G, K81D, | MRSSPGNMERIVICLMVIFLGTLVHKSSSQGQDRHMIRMRQ | 0.931 | 1392 |
| | H118D | LIDIVDQLKNYVNDLVPEFLPAPEDVETGCEWSAFSCFQDA | | |
| | | QLKSANTGNNERIINVSIKKLKRKPPSTNAGRRQKDRLTCP | | |
| | | SCDSYEKKPPKEFLERFKSLLQKMIHQHLSSRTHGSEDS | | |
| IL-21_6082 | H35F, I45T, | MRSSPGNMERIVICLMVIFLGTLVHKSSSQGQDREMIRMRQ | 0.931 | 1393 |
| | K81D, R114G | LIDTVDQLKNYVNDLVPEFLPAPEDVETNCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5627 | V98E, R114Q, | MRSSPGNMERIVICLMVIFLGTLVHKSSSQGQDRHMIRMRQ | 0.932 | 1394 |
| | R119T | LIDIVDQLKNYVNDLVPEFLPAPEDVETNCEWSAFSCFQKA | | |
| | | QLKSANTGNNERIINESIKKLKRKPPSTNAGQRQKHTLTCP | | |
| | | SCDSYEKKPPKEFLERFKSLLQKMIHQHLSSRTHGSEDS | | |
| IL-21_5904 | I45T, K81D, | MRSSPGNMERIVICLMVIFLGTLVHKSSSQGQDRHMIRMRQ | 0.933 | 1395 |
| | P107V, R115D | LIDTVDQLKNYVNDLVPEFLPAPEDVETNCEWSAFSCFQDA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_6135 | H35I, V98E, | MRSSPGNMERIVICLMVIFLGTLVHKSSSQGQDRIMIRMRQ | 0.933 | 1396 |
| | P107V, R115G | LIDIVDQLKNYVNDLVPEFLPAPEDVETNCEWSAFSCFQKA | | |
| | | QLKSANTGNNERIINESIKKLKRKVPSTNAGRGQKHRLTCP | | |
| | | SCDSYEKKPPKEFLERFKSLLQKMIHQHLSSRTHGSEDS | | |
| IL-21_5677 | K81D, A112P, | MRSSPGNMERIVICLMVIFLGTLVHKSSSQGQDRHMIRMRQ | 0.933 | 1397 |
| | R115G | LIDIVDQLKNYVNDLVPEFLPAPEDVETNCEWSAFSCFQDA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5592 | P107V, R114G, | MRSSPGNMERIVICLMVIFLGTLVHKSSSQGQDRHMIRMRQ | 0.934 | 1398 |
| | R115D | LIDIVDQLKNYVNDLVPEFLPAPEDVETNCEWSAFSCFQKA | | |
| | | QLKSANTGNNERIINVSIKKLKRKVPSTNAGGDQKHRLTCP | | |
| | | SCDSYEKKPPKEFLERFKSLLQKMIHQHLSSRTHGSEDS | | |
| IL-21_5761 | N70G, K81D, | MRSSPGNMERIVICLMVIFLGTLVHKSSSQGQDRHMIRMRQ | 0.934 | 1399 |
| | T121E | LIDIVDQLKNYVNDLVPEFLPAPEDVETGCEWSAFSCFQDA | | |
| | | QLKSANTGNNERIINVSIKKLKRKPPSTNAGRRQKHRLECP | | |
| | | SCDSYEKKPPKEFLERFKSLLQKMIHQHLSSRTHGSEDS | | |
| IL-21_5039 | P107V, T110D, | MRSSPGNMERIVICLMVIFLGTLVHKSSSQGQDRHMIRMRQ | 0.935 | 1400 |
| | R115D, R119T | LIDIVDQLKNYVNDLVPEFLPAPEDVETNCEWSAFSCFQKA | | |
| | | QLKSANTGNNERIINVSIKKLKRKVPSDNAGRDQKHTLTCP | | |
| | | SCDSYEKKPPKEFLERFKSLLQKMIHQHLSSRTHGSEDS | | |
| IL-21_5617 | V98E, R115G, | MRSSPGNMERIVICLMVIFLGTLVHKSSSQGQDRHMIRMRQ | 0.935 | 1401 |
| | H118D | LIDIVDQLKNYVNDLVPEFLPAPEDVETNCEWSAFSCFQKA | | |
| | | QLKSANTGNNERIINESIKKLKRKPPSTNAGRGQKDRLTCP | | |
| | | SCDSYEKKPPKEFLERFKSLLQKMIHQHLSSRTHGSEDS | | |
| IL-21_5986 | H35F, V98E, | MRSSPGNMERIVICLMVIFLGTLVHKSSSQGQDRFMIRMRQ | 0.936 | 1402 |
| | T110D, R114Q | LIDIVDQLKNYVNDLVPEFLPAPEDVETNCEWSAFSCFQKA | | |
| | | QLKSANTGNNERIINESIKKLKRKPPSDNAGQRQKHRLTCP | | |
| | | SCDSYEKKPPKEFLERFKSLLQKMIHQHLSSRTHGSEDS | | |
| IL-21_5133 | V98E, P107V, | MRSSPGNMERIVICLMVIFLGTLVHKSSSQGQDRHMIRMRQ | 0.937 | 1403 |
| | R115G, H118D | LIDIVDQLKNYVNDLVPEFLPAPEDVETNCEWSAFSCFQKA | | |
| | | QLKSANTGNNERIINESIKKLKRKVPSTNAGRGQKDRLTCP | | |
| | | SCDSYEKKPPKEFLERFKSLLQKMIHQHLSSRTHGSEDS | | |
| IL-21_5671 | K81D, R114Q, | MRSSPGNMERIVICLMVIFLGTLVHKSSSQGQDRHMIRMRQ | 0.937 | 1404 |
| | H118D | LIDIVDQLKNYVNDLVPEFLPAPEDVETNCEWSAFSCFQDA | | |
| | | QLKSANTGNNERIINVSIKKLKRKPPSTNAGQRQKDRLTCP | | |
| | | SCDSYEKKPPKEFLERFKSLLQKMIHQHLSSRTHGSEDS | | |
| IL-21_5622 | V98E, R114G, | MRSSPGNMERIVICLMVIFLGTLVHKSSSQGQDRHMIRMRQ | 0.938 | 1405 |
| | R119T | LIDIVDQLKNYVNDLVPEFLPAPEDVETNCEWSAFSCFQKA | | |
| | | QLKSANTGNNERIINESIKKLKRKPPSTNAGGRQKHTLTCP | | |
| | | SCDSYEKKPPKEFLERFKSLLQKMIHQHLSSRTHGSEDS | | |
| IL-21_6137 | H35I, V98E, | MRSSPGNMERIVICLMVIFLGTLVHKSSSQGQDRIMIRMRQ | 0.939 | 1406 |
| | P107V, R114G | LIDIVDQLKNYVNDLVPEFLPAPEDVETNCEWSAFSCFQKA | | |
| | | QLKSANTGNNERIINESIKKLKRKVPSTNAGGRQKHRLTCP | | |
| | | SCDSYEKKPPKEFLERFKSLLQKMIHQHLSSRTHGSEDS | | |
| IL-21_5332 | N70G, R114Q, | MRSSPGNMERIVICLMVIFLGTLVHKSSSQGQDRHMIRMRQ | 0.94 | 1407 |
| | R115D, R119T | LIDIVDQLKNYVNDLVPEFLPAPEDVETGCEWSAFSCFQKA | | |
| | | QLKSANTGNNERIINVSIKKLKRKPPSTNAGQDQKHTLTCP | | |
| | | SCDSYEKKPPKEFLERFKSLLQKMIHQHLSSRTHGSEDS | | |
| IL-21_5608 | P107V, T110D, | MRSSPGNMERIVICLMVIFLGTLVHKSSSQGQDRHMIRMRQ | 0.941 | 1408 |
| | R115D | LIDIVDQLKNYVNDLVPEFLPAPEDVETNCEWSAFSCFQKA | | |
| | | QLKSANTGNNERIINVSIKKLKRKVPSDNAGRDQKHRLTCP | | |
| | | SCDSYEKKPPKEFLERFKSLLQKMIHQHLSSRTHGSEDS | | |
| IL-21_5126 | V98E, T110D, | MRSSPGNMERIVICLMVIFLGTLVHKSSSQGQDRHMIRMRQ | 0.941 | 1409 |
| | A112P, R114G | LIDIVDQLKNYVNDLVPEFLPAPEDVETNCEWSAFSCFQKA | | |
| | | QLKSANTGNNERIINESIKKLKRKPPSDNPGGRQKHRLTCP | | |
| | | SCDSYEKKPPKEFLERFKSLLQKMIHQHLSSRTHGSEDS | | |
| IL-21_6233 | H35I, I45T, | MRSSPGNMERIVICLMVIFLGTLVHKSSSQGQDRIMIRMRQ | 0.942 | 1410 |
| | K81D, V98E | LIDTVDQLKNYVNDLVPEFLPAPEDVETNCEWSAFSCFQDA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5764 | N70G, K81D, | MRSSPGNMERIVICLMVIFLGTLVHKSSSQGQDRHMIRMRQ | 0.942 | 1411 |
| | R115G | LIDIVDQLKNYVNDLVPEFLPAPEDVETGCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5440 | N70G, V98E, | MRSSPGNMERIVICLMVIFLGTLVHKSSSQGQDRHMIRMRQ | 0.942 | 1412 |
| | T110D, R119T | LIDIVDQLKNYVNDLVPEFLPAPEDVETGCEWSAFSCFQKA | | |
| | | QLKSANTGNNERIINESIKKLKRKPPSDNAGRRQKHTLTCP | | |
| | | SCDSYEKKPPKEFLERFKSLLQKMIHQHLSSRTHGSEDS | | |
| IL-21_5149 | V98E, P107V, | MRSSPGNMERIVICLMVIFLGTLVHKSSSQGQDRHMIRMRQ | 0.943 | 1413 |
| | A112P, H118D | LIDIVDQLKNYVNDLVPEFLPAPEDVETNCEWSAFSCFQKA | | |
| | | QLKSANTGNNERIINESIKKLKRKVPSTNPGRRQKDRLTCP | | |
| | | SCDSYEKKPPKEFLERFKSLLQKMIHQHLSSRTHGSEDS | | |
| IL-21_5993 | H35F, V98E, | MRSSPGNMERIVICLMVIFLGTLVHKSSSQGQDRFMIRMRQ | 0.944 | 1414 |
| | P107V, T110D | LIDIVDQLKNYVNDLVPEFLPAPEDVETNCEWSAFSCFQKA | | |
| | | QLKSANTGNNERIINESIKKLKRKVPSDNAGRRQKHRLTCP | | |
| | | SCDSYEKKPPKEFLERFKSLLQKMIHQHLSSRTHGSEDS | | |
| IL-21_6138 | H35I, V98E, | MRSSPGNMERIVICLMVIFLGTLVHKSSSQGQDRIMIRMRQ | 0.944 | 1415 |
| | P107V, R114Q | LIDIVDQLKNYVNDLVPEFLPAPEDVETNCEWSAFSCFQKA | | |
| | | QLKSANTGNNERIINESIKKLKRKVPSTNAGQRQKHRLTCP | | |
| | | SCDSYEKKPPKEFLERFKSLLQKMIHQHLSSRTHGSEDS | | |
| IL-21_5635 | V98E, A112P, | MRSSPGNMERIVICLMVIFLGTLVHKSSSQGQDRHMIRMRQ | 0.946 | 1416 |
| | R115D | LIDIVDQLKNYVNDLVPEFLPAPEDVETNCEWSAFSCFQKA | | |
| | | QLKSANTGNNERIINESIKKLKRKPPSTNPGRDQKHRLTCP | | |
| | | SCDSYEKKPPKEFLERFKSLLQKMIHQHLSSRTHGSEDS | | |
| IL-21_5455 | N70G, V98E, | MRSSPGNMERIVICLMVIFLGTLVHKSSSQGQDRHMIRMRQ | 0.949 | 1417 |
| | P107V, T110D | LIDIVDQLKNYVNDLVPEFLPAPEDVETGCEWSAFSCFQKA | | |
| | | QLKSANTGNNERIINESIKKLKRKVPSDNAGRRQKHRLTCP | | |
| | | SCDSYEKKPPKEFLERFKSLLQKMIHQHLSSRTHGSEDS | | |
| IL-21_5990 | H35F, V98E, | MRSSPGNMERIVICLMVIFLGTLVHKSSSQGQDRFMIRMRQ | 0.95 | 1418 |
| | P107V, R114G | LIDIVDQLKNYVNDLVPEFLPAPEDVETNCEWSAFSCFQKA | | |
| | | QLKSANTGNNERIINESIKKLKRKVPSTNAGGRQKHRLTCP | | |
| | | SCDSYEKKPPKEFLERFKSLLQKMIHQHLSSRTHGSEDS | | |
| IL-21_5172 | K81D, R114G, | MRSSPGNMERIVICLMVIFLGTLVHKSSSQGQDRHMIRMRQ | 0.951 | 1419 |
| | R115G, T121E | LIDIVDQLKNYVNDLVPEFLPAPEDVETNCEWSAFSCFQDA | | |
| | | QLKSANTGNNERIINVSIKKLKRKPPSTNAGGGQKHRLECP | | |
| | | SCDSYEKKPPKEFLERFKSLLQKMIHQHLSSRTHGSEDS | | |
| IL-21_6263 | I45T, V98E, | MRSSPGNMERIVICLMVIFLGTLVHKSSSQGQDRHMIRMRQ | 0.953 | 1420 |
| | R115D | LIDTVDQLKNYVNDLVPEFLPAPEDVETNCEWSAFSCFQKA | | |
| | | QLKSANTGNNERIINESIKKLKRKPPSTNAGRDQKHRLTCP | | |
| | | SCDSYEKKPPKEFLERFKSLLQKMIHQHLSSRTHGSEDS | | |
| IL-21_5666 | K81D, R114G, | MRSSPGNMERIVICLMVIFLGTLVHKSSSQGQDRHMIRMRQ | 0.954 | 1421 |
| | H118D | LIDIVDQLKNYVNDLVPEFLPAPEDVETNCEWSAFSCFQDA | | |
| | | QLKSANTGNNERIINVSIKKLKRKPPSTNAGGRQKDRLTCP | | |
| | | SCDSYEKKPPKEFLERFKSLLQKMIHQHLSSRTHGSEDS | | |
| IL-21_5093 | V98E, A112P, | MRSSPGNMERIVICLMVIFLGTLVHKSSSQGQDRHMIRMRQ | 0.955 | 1422 |
| | R114G, R119T | LIDIVDQLKNYVNDLVPEFLPAPEDVETNCEWSAFSCFQKA | | |
| | | QLKSANTGNNERIINESIKKLKRKPPSTNPGGRQKHTLTCP | | |
| | | SCDSYEKKPPKEFLERFKSLLQKMIHQHLSSRTHGSEDS | | |
| IL-21_5257 | K81D, P107V, | MRSSPGNMERIVICLMVIFLGTLVHKSSSQGQDRHMIRMRQ | 0.956 | 1423 |
| | A112P, R114Q | LIDIVDQLKNYVNDLVPEFLPAPEDVETNCEWSAFSCFQDA | | |
| | | QLKSANTGNNERIINVSIKKLKRKVPSTNPGQRQKHRLTCP | | |
| | | SCDSYEKKPPKEFLERFKSLLQKMIHQHLSSRTHGSEDS | | |
| IL-21_5753 | N70G, V98E, | MRSSPGNMERIVICLMVIFLGTLVHKSSSQGQDRHMIRMRQ | 0.956 | 1424 |
| | H118D | LIDIVDQLKNYVNDLVPEFLPAPEDVETGCEWSAFSCFQKA | | |
| | | QLKSANTGNNERIINESIKKLKRKPPSTNAGRRQKDRLTCP | | |
| | | SCDSYEKKPPKEFLERFKSLLQKMIHQHLSSRTHGSEDS | | |
| IL-21_6364 | H35I, K81D, | MRSSPGNMERIVICLMVIFLGTLVHKSSSQGQDRIMIRMRQ | 0.957 | 1425 |
| | R115G | LIDIVDQLKNYVNDLVPEFLPAPEDVETNCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5613 | V98E, H118D, | MRSSPGNMERIVICLMVIFLGTLVHKSSSQGQDRHMIRMRQ | 0.957 | 1426 |
| | T121E | LIDIVDQLKNYVNDLVPEFLPAPEDVETNCEWSAFSCFQKA | | |
| | | QLKSANTGNNERIINESIKKLKRKPPSTNAGRRQKDRLECP | | |
| | | SCDSYEKKPPKEFLERFKSLLQKMIHQHLSSRTHGSEDS | | |
| IL-21_6006 | H35F, K81D, | MRSSPGNMERIVICLMVIFLGTLVHKSSSQGQDRFMIRMRQ | 0.958 | 1427 |
| | T110D, A112P | LIDIVDQLKNYVNDLVPEFLPAPEDVETNCEWSAFSCFQDA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5098 | V98E, A112P, | MRSSPGNMERIVICLMVIFLGTLVHKSSSQGQDRHMIRMRQ | 0.958 | 1428 |
| | R114Q, R119T | LIDIVDQLKNYVNDLVPEFLPAPEDVETNCEWSAFSCFQKA | | |
| | | QLKSANTGNNERIINESIKKLKRKPPSTNPGQRQKHTLTCP | | |
| | | SCDSYEKKPPKEFLERFKSLLQKMIHQHLSSRTHGSEDS | | |
| IL-21_5183 | K81D, R114Q, | MRSSPGNMERIVICLMVIFLGTLVHKSSSQGQDRHMIRMRQ | 0.958 | 1429 |
| | R115G, H118D | LIDIVDQLKNYVNDLVPEFLPAPEDVETNCEWSAFSCFQDA | | |
| | | QLKSANTGNNERIINVSIKKLKRKPPSTNAGQGQKDRLTCP | | |
| | | SCDSYEKKPPKEFLERFKSLLQKMIHQHLSSRTHGSEDS | | |
| IL-21_6231 | H35I, I45T, | MRSSPGNMERIVICLMVIFLGTLVHKSSSQGQDRIMIRMRQ | 0.958 | 1430 |
| | K81D, T110D | LIDTVDQLKNYVNDLVPEFLPAPEDVETNCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5480 | N70G, K81D, | MRSSPGNMERIVICLMVIFLGTLVHKSSSQGQDRHMIRMRQ | 0.958 | 1431 |
| | A112P, R114G | LIDIVDQLKNYVNDLVPEFLPAPEDVETGCEWSAFSCFQDA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5237 | K81D, P107V, | MRSSPGNMERIVICLMVIFLGTLVHKSSSQGQDRHMIRMRQ | 0.959 | 1432 |
| | R115G, H118D | LIDIVDQLKNYVNDLVPEFLPAPEDVETNCEWSAFSCFQDA | | |
| | | QLKSANTGNNERIINVSIKKLKRKVPSTNAGRGQKDRLTCP | | |
| | | SCDSYEKKPPKEFLERFKSLLQKMIHQHLSSRTHGSEDS | | |
| IL-21_4918 | R114Q, R115D, | MRSSPGNMERIVICLMVIFLGTLVHKSSSQGQDRHMIRMRQ | 0.959 | 1433 |
| | H118D, R119T | LIDIVDQLKNYVNDLVPEFLPAPEDVETNCEWSAFSCFQKA | | |
| | | QLKSANTGNNERIINVSIKKLKRKPPSTNAGQDQKDTLTCP | | |
| | | SCDSYEKKPPKEFLERFKSLLQKMIHQHLSSRTHGSEDS | | |
| IL-21_6055 | H35F, I45T, | MRSSPGNMERIVICLMVIFLGTLVHKSSSQGQDRFMIRMRQ | 0.96 | 1434 |
| | R114G, R115D | LIDTVDQLKNYVNDLVPEFLPAPEDVETNCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5827 | K81D, R115G | MRSSPGNMERIVICLMVIFLGTLVHKSSSQGQDRHMIRMRQ | 0.961 | 1435 |
| | | LIDIVDQLKNYVNDLVPEFLPAPEDVETNCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5658 | K81D, R115G, | MRSSPGNMERIVICLMVIFLGTLVHKSSSQGQDRHMIRMRQ | 0.963 | 1436 |
| | T121E | LIDIVDQLKNYVNDLVPEFLPAPEDVETNCEWSAFSCFQDA | | |
| | | QLKSANTGNNERIINVSIKKLKRKPPSTNAGRGQKHRLECP | | |
| | | SCDSYEKKPPKEFLERFKSLLQKMIHQHLSSRTHGSEDS | | |
| IL-21_5152 | V98E, P107V, | MRSSPGNMERIVICLMVIFLGTLVHKSSSQGQDRHMIRMRQ | 0.966 | 1437 |
| | A112P, R114G | LIDIVDQLKNYVNDLVPEFLPAPEDVETNCEWSAFSCFQKA | | |
| | | QLKSANTGNNERIINESIKKLKRKVPSTNPGGRQKHRLTCP | | |
| | | SCDSYEKKPPKEFLERFKSLLQKMIHQHLSSRTHGSEDS | | |
| IL-21_6224 | H35I, I45T, | MRSSPGNMERIVICLMVIFLGTLVHKSSSQGQDRIMIRMRQ | 0.966 | 1438 |
| | V98E, T110D | LIDTVDQLKNYVNDLVPEFLPAPEDVETNCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_4904 | R115D, H118D, | MRSSPGNMERIVICLMVIFLGTLVHKSSSQGQDRHMIRMRQ | 0.967 | 1439 |
| | R119T, T121E | LIDIVDQLKNYVNDLVPEFLPAPEDVETNCEWSAFSCFQKA | | |
| | | QLKSANTGNNERIINVSIKKLKRKPPSTNAGRDQKDTLECP | | |
| | | SCDSYEKKPPKEFLERFKSLLQKMIHQHLSSRTHGSEDS | | |
| IL-21_5912 | I45T, K81D, | MRSSPGNMERIVICLMVIFLGTLVHKSSSQGQDRHMIRMRQ | 0.967 | 1440 |
| | V98E, R114Q | LIDTVDQLKNYVNDLVPEFLPAPEDVETNCEWSAFSCFQDA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_6317 | H35F, K81D, | MRSSPGNMERIVICLMVIFLGTLVHKSSSQGQDRFMIRMRQ | 0.968 | 1441 |
| | P107V | LIDIVDQLKNYVNDLVPEFLPAPEDVETNCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5182 | K81D, R114Q, | MRSSPGNMERIVICLMVIFLGTLVHKSSSQGQDRHMIRMRQ | 0.969 | 1442 |
| | R115G, R119T | LIDIVDQLKNYVNDLVPEFLPAPEDVETNCEWSAFSCFQDA | | |
| | | QLKSANTGNNERIINVSIKKLKRKPPSTNAGQGQKHTLTCP | | |
| | | SCDSYEKKPPKEFLERFKSLLQKMIHQHLSSRTHGSEDS | | |
| IL-21_5905 | I45T, K81D, | MRSSPGNMERIVICLMVIFLGTLVHKSSSQGQDRHMIRMRQ | 0.972 | 1443 |
| | P107V, R114G | LIDTVDQLKNYVNDLVPEFLPAPEDVETNCEWSAFSCFQDA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_6021 | H35F, N70G, | MRSSPGNMERIVICLMVIFLGTLVHKSSSQGQDRFMIRMRQ | 0.974 | 1444 |
| | R114G, R115D | LIDIVDQLKNYVNDLVPEFLPAPEDVETGCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5253 | K81D, P107V, | MRSSPGNMERIVICLMVIFLGTLVHKSSSQGQDRHMIRMRQ | 0.974 | 1445 |
| | A112P, H118D | LIDIVDQLKNYVNDLVPEFLPAPEDVETNCEWSAFSCFQDA | | |
| | | QLKSANTGNNERIINVSIKKLKRKVPSTNPGRRQKDRLTCP | | |
| | | SCDSYEKKPPKEFLERFKSLLQKMIHQHLSSRTHGSEDS | | |
| IL-21_6156 | H35I, K81D, | MRSSPGNMERIVICLMVIFLGTLVHKSSSQGQDRIMIRMRQ | 0.976 | 1446 |
| | P107V, R114G | LIDIVDQLKNYVNDLVPEFLPAPEDVETNCEWSAFSCFQDA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5471 | N70G, K81D, | MRSSPGNMERIVICLMVIFLGTLVHKSSSQGQDRHMIRMRQ | 0.976 | 1447 |
| | R114Q, R119T | LIDIVDQLKNYVNDLVPEFLPAPEDVETGCEWSAFSCFQDA | | |
| | | QLKSANTGNNERIINVSIKKLKRKPPSTNAGQRQKHTLTCP | | |
| | | SCDSYEKKPPKEFLERFKSLLQKMIHQHLSSRTHGSEDS | | |
| IL-21_5682 | K81D, T110D, | MRSSPGNMERIVICLMVIFLGTLVHKSSSQGQDRHMIRMRQ | 0.977 | 1448 |
| | R119T | LIDIVDQLKNYVNDLVPEFLPAPEDVETNCEWSAFSCFQDA | | |
| | | QLKSANTGNNERIINVSIKKLKRKPPSDNAGRRQKHTLTCP | | |
| | | SCDSYEKKPPKEFLERFKSLLQKMIHQHLSSRTHGSEDS | | |
| IL-21_4950 | T110D, R115D, | MRSSPGNMERIVICLMVIFLGTLVHKSSSQGQDRHMIRMRQ | 0.978 | 1449 |
| | H118D, R119T | LIDIVDQLKNYVNDLVPEFLPAPEDVETNCEWSAFSCFQKA | | |
| | | QLKSANTGNNERIINVSIKKLKRKPPSDNAGRDQKDTLTCP | | |
| | | SCDSYEKKPPKEFLERFKSLLQKMIHQHLSSRTHGSEDS | | |
| IL-21_6304 | H35F, V98E, | MRSSPGNMERIVICLMVIFLGTLVHKSSSQGQDREMIRMRQ | 0.979 | 1450 |
| | R115G | LIDIVDQLKNYVNDLVPEFLPAPEDVETNCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_5754 | N70G, V98E, | MRSSPGNMERIVICLMVIFLGTLVHKSSSQGQDRHMIRMRQ | 0.981 | 1451 |
| | R115G | LIDIVDQLKNYVNDLVPEFLPAPEDVETGCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_6362 | H35I, V98E, | MRSSPGNMERIVICLMVIFLGTLVHKSSSQGQDRIMIRMRQ | 0.982 | 1452 |
| | T110D | LIDIVDQLKNYVNDLVPEFLPAPEDVETNCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5679 | K81D, A112P, | MRSSPGNMERIVICLMVIFLGTLVHKSSSQGQDRHMIRMRQ | 0.982 | 1453 |
| | R114G | LIDIVDQLKNYVNDLVPEFLPAPEDVETNCEWSAFSCFQDA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5626 | V98E, R114Q, | MRSSPGNMERIVICLMVIFLGTLVHKSSSQGQDRHMIRMRQ | 0.982 | 1454 |
| | T121E | LIDIVDQLKNYVNDLVPEFLPAPEDVETNCEWSAFSCFQKA | | |
| | | QLKSANTGNNERIINESIKKLKRKPPSTNAGQRQKHRLECP | | |
| | | SCDSYEKKPPKEFLERFKSLLQKMIHQHLSSRTHGSEDS | | |
| IL-21_5644 | V98E, T110D, | MRSSPGNMERIVICLMVIFLGTLVHKSSSQGQDRHMIRMRQ | 0.982 | 1455 |
| | R114Q | LIDIVDQLKNYVNDLVPEFLPAPEDVETNCEWSAFSCFQKA | | |
| | | QLKSANTGNNERIINESIKKLKRKPPSDNAGQRQKHRLTCP | | |
| | | SCDSYEKKPPKEFLERFKSLLQKMIHQHLSSRTHGSEDS | | |
| IL-21_5050 | P107V, T110D, | MRSSPGNMERIVICLMVIFLGTLVHKSSSQGQDRHMIRMRQ | 0.983 | 1456 |
| | R114Q, R115D | LIDIVDQLKNYVNDLVPEFLPAPEDVETNCEWSAFSCFQKA | | |
| | | QLKSANTGNNERIINVSIKKLKRKVPSDNAGQDQKHRLTCP | | |
| | | SCDSYEKKPPKEFLERFKSLLQKMIHQHLSSRTHGSEDS | | |
| IL-21_5413 | N70G, V98E, | MRSSPGNMERIVICLMVIFLGTLVHKSSSQGQDRHMIRMRQ | 0.983 | 1457 |
| | R119T, T121E | LIDIVDQLKNYVNDLVPEFLPAPEDVETGCEWSAFSCFQKA | | |
| | | QLKSANTGNNERIINESIKKLKRKPPSTNAGRRQKHTLECP | | |
| | | SCDSYEKKPPKEFLERFKSLLQKMIHQHLSSRTHGSEDS | | |
| IL-21_5705 | K81D, V98E, | MRSSPGNMERIVICLMVIFLGTLVHKSSSQGQDRHMIRMRQ | 0.983 | 1458 |
| | A112P | LIDIVDQLKNYVNDLVPEFLPAPEDVETNCEWSAFSCFQDA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_4991 | P107V, R115G, | MRSSPGNMERIVICLMVIFLGTLVHKSSSQGQDRHMIRMRQ | 0.985 | 1459 |
| | H118D, R119T | LIDIVDQLKNYVNDLVPEFLPAPEDVETNCEWSAFSCFQKA | | |
| | | QLKSANTGNNERIINVSIKKLKRKVPSTNAGRGQKDTLTCP | | |
| | | SCDSYEKKPPKEFLERFKSLLQKMIHQHLSSRTHGSEDS | | |
| IL-21_6242 | H35I, I45T, | MRSSPGNMERIVICLMVIFLGTLVHKSSSQGQDRIMIRMRQ | 0.985 | 1460 |
| | N70G, K81D | LIDTVDQLKNYVNDLVPEFLPAPEDVETGCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_4992 | P107V, R115D | MRSSPGNMERIVICLMVIFLGTLVHKSSSQGQDRHMIRMRQ | 0.985 | 1461 |
| | R119T, T121E | LIDIVDQLKNYVNDLVPEFLPAPEDVETNCEWSAFSCFQKA | | |
| | | QLKSANTGNNERIINVSIKKLKRKVPSTNAGRDQKHTLECP | | |
| | | SCDSYEKKPPKEFLERFKSLLQKMIHQHLSSRTHGSEDS | | |
| IL-21_5397 | N70G, P107V, | MRSSPGNMERIVICLMVIFLGTLVHKSSSQGQDRHMIRMRQ | 0.986 | 1462 |
| | R114Q, R115D | LIDIVDQLKNYVNDLVPEFLPAPEDVETGCEWSAFSCFQKA | | |
| | | QLKSANTGNNERIINVSIKKLKRKVPSTNAGQDQKHRLTCP | | |
| | | SCDSYEKKPPKEFLERFKSLLQKMIHQHLSSRTHGSEDS | | |
| IL-21_5892 | I45T, K81D, | MRSSPGNMERIVICLMVIFLGTLVHKSSSQGQDRHMIRMRQ | 0.987 | 1463 |
| | R114Q, R115G | LIDTVDQLKNYVNDLVPEFLPAPEDVETNCEWSAFSCFQDA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5832 | K81D, T110D | MRSSPGNMERIVICLMVIFLGTLVHKSSSQGQDRHMIRMRQ | 0.987 | 1464 |
| | | LIDIVDQLKNYVNDLVPEFLPAPEDVETNCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5202 | K81D, A112P, | MRSSPGNMERIVICLMVIFLGTLVHKSSSQGQDRHMIRMRQ | 0.988 | 1465 |
| | R114Q, R119T | LIDIVDQLKNYVNDLVPEFLPAPEDVETNCEWSAFSCFQDA | | |
| | | QLKSANTGNNERIINVSIKKLKRKPPSTNPGQRQKHTLTCP | | |
| | | SCDSYEKKPPKEFLERFKSLLQKMIHQHLSSRTHGSEDS | | |
| IL-21_6110 | H35I, P107V, | MRSSPGNMERIVICLMVIFLGTLVHKSSSQGQDRIMIRMRQ | 0.988 | 1466 |
| | R114G, R115D | LIDIVDQLKNYVNDLVPEFLPAPEDVETNCEWSAFSCFQKA | | |
| | | QLKSANTGNNERIINVSIKKLKRKVPSTNAGGDQKHRLTCP | | |
| | | SCDSYEKKPPKEFLERFKSLLQKMIHQHLSSRTHGSEDS | | |
| IL-21_5323 | N70G, R114G, | MRSSPGNMERIVICLMVIFLGTLVHKSSSQGQDRHMIRMRQ | 0.99 | 1467 |
| | R115D, R119T | LIDIVDQLKNYVNDLVPEFLPAPEDVETGCEWSAFSCFQKA | | |
| | | QLKSANTGNNERIINVSIKKLKRKPPSTNAGGDQKHTLTCP | | |
| | | SCDSYEKKPPKEFLERFKSLLQKMIHQHLSSRTHGSEDS | | |
| IL-21_6294 | H35F, T110D, | MRSSPGNMERIVICLMVIFLGTLVHKSSSQGQDREMIRMRQ | 0.99 | 1468 |
| | R115D | LIDIVDQLKNYVNDLVPEFLPAPEDVETNCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_5002 | P107V, R114G, | MRSSPGNMERIVICLMVIFLGTLVHKSSSQGQDRHMIRMRQ | 0.991 | 1469 |
| | R115D, R119T | LIDIVDQLKNYVNDLVPEFLPAPEDVETNCEWSAFSCFQKA | | |
| | | QLKSANTGNNERIINVSIKKLKRKVPSTNAGGDQKHTLTCP | | |
| | | SCDSYEKKPPKEFLERFKSLLQKMIHQHLSSRTHGSEDS | | |
| IL-21_5489 | N70G, K81D, | MRSSPGNMERIVICLMVIFLGTLVHKSSSQGQDRHMIRMRQ | 0.991 | 1470 |
| | T110D, A112P | LIDIVDQLKNYVNDLVPEFLPAPEDVETGCEWSAFSCFQDA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5153 | V98E, P107V, | MRSSPGNMERIVICLMVIFLGTLVHKSSSQGQDRHMIRMRQ | 0.991 | 1471 |
| | A112P, R114Q | LIDIVDQLKNYVNDLVPEFLPAPEDVETNCEWSAFSCFQKA | | |
| | | QLKSANTGNNERIINESIKKLKRKVPSTNPGQRQKHRLTCP | | |
| | | SCDSYEKKPPKEFLERFKSLLQKMIHQHLSSRTHGSEDS | | |
| IL-21_5690 | K81D, P107V, | MRSSPGNMERIVICLMVIFLGTLVHKSSSQGQDRHMIRMRQ | 0.993 | 1472 |
| | R119T | LIDIVDQLKNYVNDLVPEFLPAPEDVETNCEWSAFSCFQDA | | |
| | | QLKSANTGNNERIINVSIKKLKRKVPSTNAGRRQKHTLTCP | | |
| | | SCDSYEKKPPKEFLERFKSLLQKMIHQHLSSRTHGSEDS | | |
| IL-21_5429 | N70G, V98E, | MRSSPGNMERIVICLMVIFLGTLVHKSSSQGQDRHMIRMRQ | 0.993 | 1473 |
| | R114Q, H118D | LIDIVDQLKNYVNDLVPEFLPAPEDVETGCEWSAFSCFQKA | | |
| | | QLKSANTGNNERIINESIKKLKRKPPSTNAGQRQKDRLTCP | | |
| | | SCDSYEKKPPKEFLERFKSLLQKMIHQHLSSRTHGSEDS | | |
| IL-21_5895 | I45T, K81D, | MRSSPGNMERIVICLMVIFLGTLVHKSSSQGQDRHMIRMRQ | 0.993 | 1474 |
| | A112P, R115D | LIDTVDQLKNYVNDLVPEFLPAPEDVETNCEWSAFSCFQDA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5252 | K81D, P107V, | MRSSPGNMERIVICLMVIFLGTLVHKSSSQGQDRHMIRMRQ | 0.993 | 1475 |
| | A112P, R119T | LIDIVDQLKNYVNDLVPEFLPAPEDVETNCEWSAFSCFQDA | | |
| | | QLKSANTGNNERIINVSIKKLKRKVPSTNPGRRQKHTLTCP | | |
| | | SCDSYEKKPPKEFLERFKSLLQKMIHQHLSSRTHGSEDS | | |
| IL-21_5695 | K81D, P107V, | MRSSPGNMERIVICLMVIFLGTLVHKSSSQGQDRHMIRMRQ | 0.994 | 1476 |
| | R114Q | LIDIVDQLKNYVNDLVPEFLPAPEDVETNCEWSAFSCFQDA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5194 | K81D, A112P, | MRSSPGNMERIVICLMVIFLGTLVHKSSSQGQDRHMIRMRQ | 0.994 | 1477 |
| | R115D, R119T | LIDIVDQLKNYVNDLVPEFLPAPEDVETNCEWSAFSCFQDA | | |
| | | QLKSANTGNNERIINVSIKKLKRKPPSTNPGRDQKHTLTCP | | |
| | | SCDSYEKKPPKEFLERFKSLLQKMIHQHLSSRTHGSEDS | | |
| IL-21_5973 | H35F, P107V, | MRSSPGNMERIVICLMVIFLGTLVHKSSSQGQDREMIRMRQ | 0.994 | 1478 |
| | T110D, R114Q | LIDIVDQLKNYVNDLVPEFLPAPEDVETNCEWSAFSCFQKA | | |
| | | QLKSANTGNNERIINVSIKKLKRKVPSDNAGQRQKHRLTCP | | |
| | | SCDSYEKKPPKEFLERFKSLLQKMIHQHLSSRTHGSEDS | | |
| IL-21_5676 | K81D, A112P, | MRSSPGNMERIVICLMVIFLGTLVHKSSSQGQDRHMIRMRQ | 0.994 | 1479 |
| | H118D | LIDIVDQLKNYVNDLVPEFLPAPEDVETNCEWSAFSCFQDA | | |
| | | QLKSANTGNNERIINVSIKKLKRKPPSTNPGRRQKDRLTCP | | |
| | | SCDSYEKKPPKEFLERFKSLLQKMIHQHLSSRTHGSEDS | | |
| IL-21_5903 | I45T, K81D, | MRSSPGNMERIVICLMVIFLGTLVHKSSSQGQDRHMIRMRQ | 0.995 | 1480 |
| | P107V, R115G | LIDTVDQLKNYVNDLVPEFLPAPEDVETNCEWSAFSCFQDA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_6270 | I45T, K81D, | MRSSPGNMERIVICLMVIFLGTLVHKSSSQGQDRHMIRMRQ | 0.995 | 1481 |
| | R115D | LIDTVDQLKNYVNDLVPEFLPAPEDVETNCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5691 | K81D, P107V, | MRSSPGNMERIVICLMVIFLGTLVHKSSSQGQDRHMIRMRQ | 0.998 | 1482 |
| | H118D | LIDIVDQLKNYVNDLVPEFLPAPEDVETNCEWSAFSCFQDA | | |
| | | QLKSANTGNNERIINVSIKKLKRKVPSTNAGRRQKDRLTCP | | |
| | | SCDSYEKKPPKEFLERFKSLLQKMIHQHLSSRTHGSEDS | | |
| IL-21_5409 | N70G, P107V, | MRSSPGNMERIVICLMVIFLGTLVHKSSSQGQDRHMIRMRQ | 0.998 | 1483 |
| | T110D, R115D | LIDIVDQLKNYVNDLVPEFLPAPEDVETGCEWSAFSCFQKA | | |
| | | QLKSANTGNNERIINVSIKKLKRKVPSDNAGRDQKHRLTCP | | |
| | | SCDSYEKKPPKEFLERFKSLLQKMIHQHLSSRTHGSEDS | | |
| IL-21_5201 | K81D, A112P, | MRSSPGNMERIVICLMVIFLGTLVHKSSSQGQDRHMIRMRQ | 0.998 | 1484 |
| | R114Q, T121E | LIDIVDQLKNYVNDLVPEFLPAPEDVETNCEWSAFSCFQDA | | |
| | | QLKSANTGNNERIINVSIKKLKRKPPSTNPGQRQKHRLECP | | |
| | | SCDSYEKKPPKEFLERFKSLLQKMIHQHLSSRTHGSEDS | | |
| IL-21_5956 | H35F, T110D, | MRSSPGNMERIVICLMVIFLGTLVHKSSSQGQDREMIRMRQ | 0.999 | 1485 |
| | R114Q, R115G | LIDIVDQLKNYVNDLVPEFLPAPEDVETNCEWSAFSCFQKA | | |
| | | QLKSANTGNNERIINVSIKKLKRKPPSDNAGQGQKHRLTCP | | |
| | | SCDSYEKKPPKEFLERFKSLLQKMIHQHLSSRTHGSEDS | | |
| IL-21_5150 | V98E, P107V, | MRSSPGNMERIVICLMVIFLGTLVHKSSSQGQDRHMIRMRQ | 1 | 1486 |
| | A112P, R115G | LIDIVDQLKNYVNDLVPEFLPAPEDVETNCEWSAFSCFQKA | | |
| | | QLKSANTGNNERIINESIKKLKRKVPSTNPGRGQKHRLTCP | | |
| | | SCDSYEKKPPKEFLERFKSLLQKMIHQHLSSRTHGSEDS | | |
| IL-21_6170 | H35I, N70G, | MRSSPGNMERIVICLMVIFLGTLVHKSSSQGQDRIMIRMRQ | 1 | 1487 |
| | R114Q, R115D | LIDIVDQLKNYVNDLVPEFLPAPEDVETGCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_6158 | H35I, K81D, | MRSSPGNMERIVICLMVIFLGTLVHKSSSQGQDRIMIRMRQ | 1.001 | 1488 |
| | P107V, A112P | LIDIVDQLKNYVNDLVPEFLPAPEDVETNCEWSAFSCFQDA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_5853 | I45T, T110D, | MRSSPGNMERIVICLMVIFLGTLVHKSSSQGQDRHMIRMRQ | 1.001 | 1489 |
| | R114Q, R115D | LIDTVDQLKNYVNDLVPEFLPAPEDVETNCEWSAFSCFQKA | | |
| | | QLKSANTGNNERIINVSIKKLKRKPPSDNAGQDQKHRLTCP | | |
| | | SCDSYEKKPPKEFLERFKSLLQKMIHQHLSSRTHGSEDS | | |
| IL-21_5816 | V98E, H118D | MRSSPGNMERIVICLMVIFLGTLVHKSSSQGQDRHMIRMRQ | 1.001 | 1490 |
| | | LIDIVDQLKNYVNDLVPEFLPAPEDVETNCEWSAFSCFQKA | | |
| | | QLKSANTGNNERIINESIKKLKRKPPSTNAGRRQKDRLTCP | | |
| | | SCDSYEKKPPKEFLERFKSLLQKMIHQHLSSRTHGSEDS | | |
| IL-21_5312 | N70G, R115G, | MRSSPGNMERIVICLMVIFLGTLVHKSSSQGQDRHMIRMRQ | 1.004 | 1491 |
| | H118D, R119T | LIDIVDQLKNYVNDLVPEFLPAPEDVETGCEWSAFSCFQKA | | |
| | | QLKSANTGNNERIINVSIKKLKRKPPSTNAGRGQKDTLTCP | | |
| | | SCDSYEKKPPKEFLERFKSLLQKMIHQHLSSRTHGSEDS | | |
| IL-21_5615 | V98E, R115G, | MRSSPGNMERIVICLMVIFLGTLVHKSSSQGQDRHMIRMRQ | 1.004 | 1492 |
| | T121E | LIDIVDQLKNYVNDLVPEFLPAPEDVETNCEWSAFSCFQKA | | |
| | | QLKSANTGNNERIINESIKKLKRKPPSTNAGRGQKHRLECP | | |
| | | SCDSYEKKPPKEFLERFKSLLQKMIHQHLSSRTHGSEDS | | |
| IL-21_5470 | N70G, K81D, | MRSSPGNMERIVICLMVIFLGTLVHKSSSQGQDRHMIRMRQ | 1.006 | 1493 |
| | R114Q, T121E | LIDIVDQLKNYVNDLVPEFLPAPEDVETGCEWSAFSCFQDA | | |
| | | QLKSANTGNNERIINVSIKKLKRKPPSTNAGQRQKHRLECP | | |
| | | SCDSYEKKPPKEFLERFKSLLQKMIHQHLSSRTHGSEDS | | |
| IL-21_5752 | N70G, V98E, | MRSSPGNMERIVICLMVIFLGTLVHKSSSQGQDRHMIRMRQ | 1.006 | 1494 |
| | R119T | LIDIVDQLKNYVNDLVPEFLPAPEDVETGCEWSAFSCFQKA | | |
| | | QLKSANTGNNERIINESIKKLKRKPPSTNAGRRQKHTLTCP | | |
| | | SCDSYEKKPPKEFLERFKSLLQKMIHQHLSSRTHGSEDS | | |
| IL-21_6085 | H35F, I45T, | MRSSPGNMERIVICLMVIFLGTLVHKSSSQGQDRFMIRMRQ | 1.008 | 1495 |
| | K81D, T110D | LIDTVDQLKNYVNDLVPEFLPAPEDVETNCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5251 | K81D, P107V, | MRSSPGNMERIVICLMVIFLGTLVHKSSSQGQDRHMIRMRQ | 1.009 | 1496 |
| | A112P, T121E | LIDIVDQLKNYVNDLVPEFLPAPEDVETNCEWSAFSCFQDA | | |
| | | QLKSANTGNNERIINVSIKKLKRKVPSTNPGRRQKHRLECP | | |
| | | SCDSYEKKPPKEFLERFKSLLQKMIHQHLSSRTHGSEDS | | |
| IL-21_5037 | P107V, T110D, | MRSSPGNMERIVICLMVIFLGTLVHKSSSQGQDRHMIRMRQ | 1.011 | 1497 |
| | R115G, H118D | LIDIVDQLKNYVNDLVPEFLPAPEDVETNCEWSAFSCFQKA | | |
| | | QLKSANTGNNERIINVSIKKLKRKVPSDNAGRGQKDRLTCP | | |
| | | SCDSYEKKPPKEFLERFKSLLQKMIHQHLSSRTHGSEDS | | |
| IL-21_4959 | T110D, R114G, | MRSSPGNMERIVICLMVIFLGTLVHKSSSQGQDRHMIRMRQ | 1.011 | 1498 |
| | R115D, H118D | LIDIVDQLKNYVNDLVPEFLPAPEDVETNCEWSAFSCFQKA | | |
| | | QLKSANTGNNERIINVSIKKLKRKPPSDNAGGDQKDRLTCP | | |
| | | SCDSYEKKPPKEFLERFKSLLQKMIHQHLSSRTHGSEDS | | |
| IL-21_5387 | N70G, P107V, | MRSSPGNMERIVICLMVIFLGTLVHKSSSQGQDRHMIRMRQ | 1.012 | 1499 |
| | R115D, H118D | LIDIVDQLKNYVNDLVPEFLPAPEDVETGCEWSAFSCFQKA | | |
| | | QLKSANTGNNERIINVSIKKLKRKVPSTNAGRDQKDRLTCP | | |
| | | SCDSYEKKPPKEFLERFKSLLQKMIHQHLSSRTHGSEDS | | |
| IL-21_6118 | H35I, P107V, | MRSSPGNMERIVICLMVIFLGTLVHKSSSQGQDRIMIRMRQ | 1.012 | 1500 |
| | T110D, R115D | LIDIVDQLKNYVNDLVPEFLPAPEDVETNCEWSAFSCFQKA | | |
| | | QLKSANTGNNERIINVSIKKLKRKVPSDNAGRDQKHRLTCP | | |
| | | SCDSYEKKPPKEFLERFKSLLQKMIHQHLSSRTHGSEDS | | |
| IL-21_5830 | K81D, R114Q | MRSSPGNMERIVICLMVIFLGTLVHKSSSQGQDRHMIRMRQ | 1.012 | 1501 |
| | | LIDIVDQLKNYVNDLVPEFLPAPEDVETNCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_6191 | H35I, N70G, | MRSSPGNMERIVICLMVIFLGTLVHKSSSQGQDRIMIRMRQ | 1.013 | 1502 |
| | V98E, P107V | LIDIVDQLKNYVNDLVPEFLPAPEDVETGCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5159 | V98E, P107V, | MRSSPGNMERIVICLMVIFLGTLVHKSSSQGQDRHMIRMRQ | 1.013 | 1503 |
| | T110D, R114G | LIDIVDQLKNYVNDLVPEFLPAPEDVETNCEWSAFSCFQKA | | |
| | | QLKSANTGNNERIINESIKKLKRKVPSDNAGGRQKHRLTCP | | |
| | | SCDSYEKKPPKEFLERFKSLLQKMIHQHLSSRTHGSEDS | | |
| IL-21_5001 | P107V, R114G, | MRSSPGNMERIVICLMVIFLGTLVHKSSSQGQDRHMIRMRQ | 1.013 | 1504 |
| | R115D, T121E | LIDIVDQLKNYVNDLVPEFLPAPEDVETNCEWSAFSCFQKA | | |
| | | QLKSANTGNNERIINVSIKKLKRKVPSTNAGGDQKHRLECP | | |
| | | SCDSYEKKPPKEFLERFKSLLQKMIHQHLSSRTHGSEDS | | |
| IL-21_5092 | V98E, A112P, | MRSSPGNMERIVICLMVIFLGTLVHKSSSQGQDRHMIRMRQ | 1.014 | 1505 |
| | R114G, T121E | LIDIVDQLKNYVNDLVPEFLPAPEDVETNCEWSAFSCFQKA | | |
| | | QLKSANTGNNERIINESIKKLKRKPPSTNPGGRQKHRLECP | | |
| | | SCDSYEKKPPKEFLERFKSLLQKMIHQHLSSRTHGSEDS | | |
| IL-21_5360 | N70G, T110D, | MRSSPGNMERIVICLMVIFLGTLVHKSSSQGQDRHMIRMRQ | 1.014 | 1506 |
| | R115D, R119T | LIDIVDQLKNYVNDLVPEFLPAPEDVETGCEWSAFSCFQKA | | |
| | | QLKSANTGNNERIINVSIKKLKRKPPSDNAGRDQKHTLTCP | | |
| | | SCDSYEKKPPKEFLERFKSLLQKMIHQHLSSRTHGSEDS | | |
| IL-21_5943 | I45T, N70G, | MRSSPGNMERIVICLMVIFLGTLVHKSSSQGQDRHMIRMRQ | 1.015 | 1507 |
| | K81D, R115D | LIDTVDQLKNYVNDLVPEFLPAPEDVETGCEWSAFSCFQDA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5097 | V98E, A112P, | MRSSPGNMERIVICLMVIFLGTLVHKSSSQGQDRHMIRMRQ | 1.015 | 1508 |
| | R114Q, T121E | LIDIVDQLKNYVNDLVPEFLPAPEDVETNCEWSAFSCFQKA | | |
| | | QLKSANTGNNERIINESIKKLKRKPPSTNPGQRQKHRLECP | | |
| | | SCDSYEKKPPKEFLERFKSLLQKMIHQHLSSRTHGSEDS | | |
| IL-21_6222 | H351, I45T, | MRSSPGNMERIVICLMVIFLGTLVHKSSSQGQDRIMIRMRQ | 1.018 | 1509 |
| | V98E, R114Q | LIDTVDQLKNYVNDLVPEFLPAPEDVETNCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_6380 | H35I, N70G, | MRSSPGNMERIVICLMVIFLGTLVHKSSSQGQDRIMIRMRQ | 1.018 | 1510 |
| | K81D | LIDIVDQLKNYVNDLVPEFLPAPEDVETGCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5040 | P107V, T110D, | MRSSPGNMERIVICLMVIFLGTLVHKSSSQGQDRHMIRMRQ | 1.018 | 1511 |
| | R115D, H118D | LIDIVDQLKNYVNDLVPEFLPAPEDVETNCEWSAFSCFQKA | | |
| | | QLKSANTGNNERIINVSIKKLKRKVPSDNAGRDQKDRLTCP | | |
| | | SCDSYEKKPPKEFLERFKSLLQKMIHQHLSSRTHGSEDS | | |
| IL-21_5688 | K81D, T110D, | MRSSPGNMERIVICLMVIFLGTLVHKSSSQGQDRHMIRMRQ | 1.019 | 1512 |
| | A112P | LIDIVDQLKNYVNDLVPEFLPAPEDVETNCEWSAFSCFQDA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_6075 | H35F, I45T, | MRSSPGNMERIVICLMVIFLGTLVHKSSSQGQDREMIRMRQ | 1.019 | 1513 |
| | V98E, R114G | LIDTVDQLKNYVNDLVPEFLPAPEDVETNCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5639 | V98E, T110D, | MRSSPGNMERIVICLMVIFLGTLVHKSSSQGQDRHMIRMRQ | 1.02 | 1514 |
| | R119T | LIDIVDQLKNYVNDLVPEFLPAPEDVETNCEWSAFSCFQKA | | |
| | | QLKSANTGNNERIINESIKKLKRKPPSDNAGRRQKHTLTCP | | |
| | | SCDSYEKKPPKEFLERFKSLLQKMIHQHLSSRTHGSEDS | | |
| IL-21_4943 | A112P, R114Q, | MRSSPGNMERIVICLMVIFLGTLVHKSSSQGQDRHMIRMRQ | 1.02 | 1515 |
| | R115D, H118D | LIDIVDQLKNYVNDLVPEFLPAPEDVETNCEWSAFSCFQKA | | |
| | | QLKSANTGNNERIINVSIKKLKRKPPSTNPGQDQKDRLTCP | | |
| | | SCDSYEKKPPKEFLERFKSLLQKMIHQHLSSRTHGSEDS | | |
| IL-21_4957 | T110D, R114G, | MRSSPGNMERIVICLMVIFLGTLVHKSSSQGQDRHMIRMRQ | 1.02 | 1516 |
| | R115D, T121E | LIDIVDQLKNYVNDLVPEFLPAPEDVETNCEWSAFSCFQKA | | |
| | | QLKSANTGNNERIINVSIKKLKRKPPSDNAGGDQKHRLECP | | |
| | | SCDSYEKKPPKEFLERFKSLLQKMIHQHLSSRTHGSEDS | | |
| IL-21_6148 | H35I, K81D, | MRSSPGNMERIVICLMVIFLGTLVHKSSSQGQDRIMIRMRQ | 1.02 | 1517 |
| | A112P, R114Q | LIDIVDQLKNYVNDLVPEFLPAPEDVETNCEWSAFSCFQDA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5192 | K81D, A112P, | MRSSPGNMERIVICLMVIFLGTLVHKSSSQGQDRHMIRMRQ | 1.022 | 1518 |
| | R115G, H118D | LIDIVDQLKNYVNDLVPEFLPAPEDVETNCEWSAFSCFQDA | | |
| | | QLKSANTGNNERIINVSIKKLKRKPPSTNPGRGQKDRLTCP | | |
| | | SCDSYEKKPPKEFLERFKSLLQKMIHQHLSSRTHGSEDS | | |
| IL-21_6086 | H35F, I45T, | MRSSPGNMERIVICLMVIFLGTLVHKSSSQGQDREMIRMRQ | 1.027 | 1519 |
| | K81D, P107V | LIDTVDQLKNYVNDLVPEFLPAPEDVETNCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5664 | K81D, R114G, | MRSSPGNMERIVICLMVIFLGTLVHKSSSQGQDRHMIRMRQ | 1.028 | 1520 |
| | T121E | LIDIVDQLKNYVNDLVPEFLPAPEDVETNCEWSAFSCFQDA | | |
| | | QLKSANTGNNERIINVSIKKLKRKPPSTNAGGRQKHRLECP | | |
| | | SCDSYEKKPPKEFLERFKSLLQKMIHQHLSSRTHGSEDS | | |
| IL-21_5872 | I45T, V98E, | MRSSPGNMERIVICLMVIFLGTLVHKSSSQGQDRHMIRMRQ | 1.028 | 1521 |
| | R114G, R115D | LIDTVDQLKNYVNDLVPEFLPAPEDVETNCEWSAFSCFQKA | | |
| | | QLKSANTGNNERIINESIKKLKRKPPSTNAGGDQKHRLTCP | | |
| | | SCDSYEKKPPKEFLERFKSLLQKMIHQHLSSRTHGSEDS | | |
| IL-21_5456 | N70G, K81D, | MRSSPGNMERIVICLMVIFLGTLVHKSSSQGQDRHMIRMRQ | 1.028 | 1522 |
| | R119T, T121E | LIDIVDQLKNYVNDLVPEFLPAPEDVETGCEWSAFSCFQDA | | |
| | | QLKSANTGNNERIINVSIKKLKRKPPSTNAGRRQKHTLECP | | |
| | | SCDSYEKKPPKEFLERFKSLLQKMIHQHLSSRTHGSEDS | | |
| IL-21_4966 | T110D, R114Q, | MRSSPGNMERIVICLMVIFLGTLVHKSSSQGQDRHMIRMRQ | 1.029 | 1523 |
| | R115D, T121E | LIDIVDQLKNYVNDLVPEFLPAPEDVETNCEWSAFSCFQKA | | |
| | | QLKSANTGNNERIINVSIKKLKRKPPSDNAGQDQKHRLECP | | |
| | | SCDSYEKKPPKEFLERFKSLLQKMIHQHLSSRTHGSEDS | | |
| IL-21_5435 | N70G, V98E, | MRSSPGNMERIVICLMVIFLGTLVHKSSSQGQDRHMIRMRQ | 1.029 | 1524 |
| | A112P, R115G | LIDIVDQLKNYVNDLVPEFLPAPEDVETGCEWSAFSCFQKA | | |
| | | QLKSANTGNNERIINESIKKLKRKPPSTNPGRGQKHRLTCP | | |
| | | SCDSYEKKPPKEFLERFKSLLQKMIHQHLSSRTHGSEDS | | |
| IL-21_5683 | K81D, T110D, | MRSSPGNMERIVICLMVIFLGTLVHKSSSQGQDRHMIRMRQ | 1.031 | 1525 |
| | H118D | LIDIVDQLKNYVNDLVPEFLPAPEDVETNCEWSAFSCFQDA | | |
| | | QLKSANTGNNERIINVSIKKLKRKPPSDNAGRRQKDRLTCP | | |
| | | SCDSYEKKPPKEFLERFKSLLQKMIHQHLSSRTHGSEDS | | |
| IL-21_4910 | R114G, R115D, | MRSSPGNMERIVICLMVIFLGTLVHKSSSQGQDRHMIRMRQ | 1.031 | 1526 |
| | H118D, T121E | LIDIVDQLKNYVNDLVPEFLPAPEDVETNCEWSAFSCFQKA | | |
| | | QLKSANTGNNERIINVSIKKLKRKPPSTNAGGDQKDRLECP | | |
| | | SCDSYEKKPPKEFLERFKSLLQKMIHQHLSSRTHGSEDS | | |
| IL-21_5651 | V98E, P107V, | MRSSPGNMERIVICLMVIFLGTLVHKSSSQGQDRHMIRMRQ | 1.031 | 1527 |
| | R114G | LIDIVDQLKNYVNDLVPEFLPAPEDVETNCEWSAFSCFQKA | | |
| | | QLKSANTGNNERIINESIKKLKRKVPSTNAGGRQKHRLTCP | | |
| | | SCDSYEKKPPKEFLERFKSLLQKMIHQHLSSRTHGSEDS | | |
| IL-21_6309 | H35F, V98E, | MRSSPGNMERIVICLMVIFLGTLVHKSSSQGQDREMIRMRQ | 1.032 | 1528 |
| | T110D | LIDIVDQLKNYVNDLVPEFLPAPEDVETNCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5225 | K81D, T110D, | MRSSPGNMERIVICLMVIFLGTLVHKSSSQGQDRHMIRMRQ | 1.032 | 1529 |
| | A112P, T121E | LIDIVDQLKNYVNDLVPEFLPAPEDVETNCEWSAFSCFQDA | | |
| | | QLKSANTGNNERIINVSIKKLKRKPPSDNPGRRQKHRLECP | | |
| | | SCDSYEKKPPKEFLERFKSLLQKMIHQHLSSRTHGSEDS | | |
| IL-21_5189 | K81D, A112P, | MRSSPGNMERIVICLMVIFLGTLVHKSSSQGQDRHMIRMRQ | 1.032 | 1530 |
| | H118D, R119T | LIDIVDQLKNYVNDLVPEFLPAPEDVETNCEWSAFSCFQDA | | |
| | | QLKSANTGNNERIINVSIKKLKRKPPSTNPGRRQKDTLTCP | | |
| | | SCDSYEKKPPKEFLERFKSLLQKMIHQHLSSRTHGSEDS | | |
| IL-21_5003 | P107V, R114G, | MRSSPGNMERIVICLMVIFLGTLVHKSSSQGQDRHMIRMRQ | 1.034 | 1531 |
| | R115D, H118D | LIDIVDQLKNYVNDLVPEFLPAPEDVETNCEWSAFSCFQKA | | |
| | | QLKSANTGNNERIINVSIKKLKRKVPSTNAGGDQKDRLTCP | | |
| | | SCDSYEKKPPKEFLERFKSLLQKMIHQHLSSRTHGSEDS | | |
| IL-21_6096 | H35F, I45T, | MRSSPGNMERIVICLMVIFLGTLVHKSSSQGQDRFMIRMRQ | 1.034 | 1532 |
| | N70G, K81D | LIDTVDQLKNYVNDLVPEFLPAPEDVETGCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6169 | H35I, N70G, | MRSSPGNMERIVICLMVIFLGTLVHKSSSQGQDRIMIRMRQ | 1.034 | 1533 |
| | R114Q, R115G | LIDIVDQLKNYVNDLVPEFLPAPEDVETGCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_6196 | H35I, N70G, | MRSSPGNMERIVICLMVIFLGTLVHKSSSQGQDRIMIRMRQ | 1.035 | 1534 |
| | K81D, A112P | LIDIVDQLKNYVNDLVPEFLPAPEDVETGCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5370 | N70G, T110D, | MRSSPGNMERIVICLMVIFLGTLVHKSSSQGQDRHMIRMRQ | 1.035 | 1535 |
| | R114Q, R115G | LIDIVDQLKNYVNDLVPEFLPAPEDVETGCEWSAFSCFQKA | | |
| | | QLKSANTGNNERIINVSIKKLKRKPPSDNAGQGQKHRLTCP | | |
| | | SCDSYEKKPPKEFLERFKSLLQKMIHQHLSSRTHGSEDS | | |
| IL-21_5829 | K81D, R114G | MRSSPGNMERIVICLMVIFLGTLVHKSSSQGQDRHMIRMRQ | 1.036 | 1536 |
| | | LIDIVDQLKNYVNDLVPEFLPAPEDVETNCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_6192 | H35I, N70G, | MRSSPGNMERIVICLMVIFLGTLVHKSSSQGQDRIMIRMRQ | 1.036 | 1537 |
| | K81D, R115G | LIDIVDQLKNYVNDLVPEFLPAPEDVETGCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5256 | K81D, P107V, | MRSSPGNMERIVICLMVIFLGTLVHKSSSQGQDRHMIRMRQ | 1.038 | 1538 |
| | A112P, R114G | LIDIVDQLKNYVNDLVPEFLPAPEDVETNCEWSAFSCFQDA | | |
| | | QLKSANTGNNERIINVSIKKLKRKVPSTNPGGRQKHRLTCP | | |
| | | SCDSYEKKPPKEFLERFKSLLQKMIHQHLSSRTHGSEDS | | |
| IL-21_6084 | H35F, I45T, | MRSSPGNMERIVICLMVIFLGTLVHKSSSQGQDRFMIRMRQ | 1.039 | 1539 |
| | K81D, A112P | LIDTVDQLKNYVNDLVPEFLPAPEDVETNCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5945 | I45T, N70G, | MRSSPGNMERIVICLMVIFLGTLVHKSSSQGQDRHMIRMRQ | 1.042 | 1540 |
| | K81D, R114Q | LIDTVDQLKNYVNDLVPEFLPAPEDVETGCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5770 | N70G, K81D, | MRSSPGNMERIVICLMVIFLGTLVHKSSSQGQDRHMIRMRQ | 1.043 | 1541 |
| | P107V | LIDIVDQLKNYVNDLVPEFLPAPEDVETGCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5178 | K81D, R114Q, | MRSSPGNMERIVICLMVIFLGTLVHKSSSQGQDRHMIRMRQ | 1.045 | 1542 |
| | R119T, T121E | LIDIVDQLKNYVNDLVPEFLPAPEDVETNCEWSAFSCFQDA | | |
| | | QLKSANTGNNERIINVSIKKLKRKPPSTNAGQRQKHTLECP | | |
| | | SCDSYEKKPPKEFLERFKSLLQKMIHQHLSSRTHGSEDS | | |
| IL-21_6023 | H35F, N70G, | MRSSPGNMERIVICLMVIFLGTLVHKSSSQGQDREMIRMRQ | 1.047 | 1543 |
| | R114Q, R115D | LIDIVDQLKNYVNDLVPEFLPAPEDVETGCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5475 | N70G, K81D, | MRSSPGNMERIVICLMVIFLGTLVHKSSSQGQDRHMIRMRQ | 1.048 | 1544 |
| | A112P, T121E | LIDIVDQLKNYVNDLVPEFLPAPEDVETGCEWSAFSCFQDA | | |
| | | QLKSANTGNNERIINVSIKKLKRKPPSTNPGRRQKHRLECP | | |
| | | SCDSYEKKPPKEFLERFKSLLQKMIHQHLSSRTHGSEDS | | |
| IL-21_5433 | N70G, V98E, | MRSSPGNMERIVICLMVIFLGTLVHKSSSQGQDRHMIRMRQ | 1.048 | 1545 |
| | A112P, R119T | LIDIVDQLKNYVNDLVPEFLPAPEDVETGCEWSAFSCFQKA | | |
| | | QLKSANTGNNERIINESIKKLKRKPPSTNPGRRQKHTLTCP | | |
| | | SCDSYEKKPPKEFLERFKSLLQKMIHQHLSSRTHGSEDS | | |
| IL-21_5637 | V98E, A112P, | MRSSPGNMERIVICLMVIFLGTLVHKSSSQGQDRHMIRMRQ | 1.048 | 1546 |
| | R114Q | LIDIVDQLKNYVNDLVPEFLPAPEDVETNCEWSAFSCFQKA | | |
| | | QLKSANTGNNERIINESIKKLKRKPPSTNPGQRQKHRLTCP | | |
| | | SCDSYEKKPPKEFLERFKSLLQKMIHQHLSSRTHGSEDS | | |
| IL-21_4917 | R114Q, R115, | MRSSPGNMERIVICLMVIFLGTLVHKSSSQGQDRHMIRMRQ | 1.049 | 1547 |
| | H118D, T121E | LIDIVDQLKNYVNDLVPEFLPAPEDVETNCEWSAFSCFQKA | | |
| | | QLKSANTGNNERIINVSIKKLKRKPPSTNAGQDQKDRLECP | | |
| | | SCDSYEKKPPKEFLERFKSLLQKMIHQHLSSRTHGSEDS | | |
| IL-21_5645 | V98E, T110D, | MRSSPGNMERIVICLMVIFLGTLVHKSSSQGQDRHMIRMRQ | 1.052 | 1548 |
| | A112P | LIDIVDQLKNYVNDLVPEFLPAPEDVETNCEWSAFSCFQKA | | |
| | | QLKSANTGNNERIINESIKKLKRKPPSDNPGRRQKHRLTCP | | |
| | | SCDSYEKKPPKEFLERFKSLLQKMIHQHLSSRTHGSEDS | | |
| IL-21_5196 | K81D, A112P, | MRSSPGNMERIVICLMVIFLGTLVHKSSSQGQDRHMIRMRQ | 1.052 | 1549 |
| | R114G, T121E | LIDIVDQLKNYVNDLVPEFLPAPEDVETNCEWSAFSCFQDA | | |
| | | QLKSANTGNNERIINVSIKKLKRKPPSTNPGGRQKHRLECP | | |
| | | SCDSYEKKPPKEFLERFKSLLQKMIHQHLSSRTHGSEDS | | |
| IL-21_5322 | N70G, R114G, | MRSSPGNMERIVICLMVIFLGTLVHKSSSQGQDRHMIRMRQ | 1.052 | 1550 |
| | R115D, T121E | LIDIVDQLKNYVNDLVPEFLPAPEDVETGCEWSAFSCFQKA | | |
| | | QLKSANTGNNERIINVSIKKLKRKPPSTNAGGDQKHRLECP | | |
| | | SCDSYEKKPPKEFLERFKSLLQKMIHQHLSSRTHGSEDS | | |
| IL-21_5448 | N70G, V98E, | MRSSPGNMERIVICLMVIFLGTLVHKSSSQGQDRHMIRMRQ | 1.053 | 1551 |
| | P107V, R119T | LIDIVDQLKNYVNDLVPEFLPAPEDVETGCEWSAFSCFQKA | | |
| | | QLKSANTGNNERIINESIKKLKRKVPSTNAGRRQKHTLTCP | | |
| | | SCDSYEKKPPKEFLERFKSLLQKMIHQHLSSRTHGSEDS | | |
| IL-21_5587 | P107V, R115D, | MRSSPGNMERIVICLMVIFLGTLVHKSSSQGQDRHMIRMRQ | 1.054 | 1552 |
| | H118D | LIDIVDQLKNYVNDLVPEFLPAPEDVETNCEWSAFSCFQKA | | |
| | | QLKSANTGNNERIINVSIKKLKRKVPSTNAGRDQKDRLTCP | | |
| | | SCDSYEKKPPKEFLERFKSLLQKMIHQHLSSRTHGSEDS | | |
| IL-21_5447 | N70G, V98E, | MRSSPGNMERIVICLMVIFLGTLVHKSSSQGQDRHMIRMRQ | 1.054 | 1553 |
| | P107V, T121E | LIDIVDQLKNYVNDLVPEFLPAPEDVETGCEWSAFSCFQKA | | |
| | | QLKSANTGNNERIINESIKKLKRKVPSTNAGRRQKHRLECP | | |
| | | SCDSYEKKPPKEFLERFKSLLQKMIHQHLSSRTHGSEDS | | |
| IL-21_5026 | P107V, A112P, | MRSSPGNMERIVICLMVIFLGTLVHKSSSQGQDRHMIRMRQ | 1.055 | 1554 |
| | R114G, R115D | LIDIVDQLKNYVNDLVPEFLPAPEDVETNCEWSAFSCFQKA | | |
| | | QLKSANTGNNERIINVSIKKLKRKVPSTNPGGDQKHRLTCP | | |
| | | SCDSYEKKPPKEFLERFKSLLQKMIHQHLSSRTHGSEDS | | |
| IL-21_6175 | H35I, N70G, | MRSSPGNMERIVICLMVIFLGTLVHKSSSQGQDRIMIRMRQ | 1.055 | 1555 |
| | T110D, R115G | LIDIVDQLKNYVNDLVPEFLPAPEDVETGCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_6194 | H35I, N70G, | MRSSPGNMERIVICLMVIFLGTLVHKSSSQGQDRIMIRMRQ | 1.055 | 1556 |
| | K81D, R114G | LIDIVDQLKNYVNDLVPEFLPAPEDVETGCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5766 | N70G, K81D, | MRSSPGNMERIVICLMVIFLGTLVHKSSSQGQDRHMIRMRQ | 1.056 | 1557 |
| | R114G | LIDIVDQLKNYVNDLVPEFLPAPEDVETGCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5726 | N70G, R114Q, | MRSSPGNMERIVICLMVIFLGTLVHKSSSQGQDRHMIRMRQ | 1.056 | 1558 |
| | R115D | LIDIVDQLKNYVNDLVPEFLPAPEDVETGCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5944 | I45T, N70G, | MRSSPGNMERIVICLMVIFLGTLVHKSSSQGQDRHMIRMRQ | 1.057 | 1559 |
| | K81D, R114G | LIDTVDQLKNYVNDLVPEFLPAPEDVETGCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_4997 | P107V, R114G, | MRSSPGNMERIVICLMVIFLGTLVHKSSSQGQDRHMIRMRQ | 1.057 | 1560 |
| | H118D, R119T | LIDIVDQLKNYVNDLVPEFLPAPEDVETNCEWSAFSCFQKA | | |
| | | QLKSANTGNNERIINVSIKKLKRKVPSTNAGGRQKDTLTCP | | |
| | | SCDSYEKKPPKEFLERFKSLLQKMIHQHLSSRTHGSEDS | | |
| IL-21_5982 | H35F, V98E, | MRSSPGNMERIVICLMVIFLGTLVHKSSSQGQDREMIRMRQ | 1.058 | 1561 |
| | A112P, R114Q | LIDIVDQLKNYVNDLVPEFLPAPEDVETNCEWSAFSCFQKA | | |
| | | QLKSANTGNNERIINESIKKLKRKPPSTNPGQRQKHRLTCP | | |
| | | SCDSYEKKPPKEFLERFKSLLQKMIHQHLSSRTHGSEDS | | |
| IL-21_5942 | I45T, N70G, | MRSSPGNMERIVICLMVIFLGTLVHKSSSQGQDRHMIRMRQ | 1.058 | 1562 |
| | K81D, R115G | LIDTVDQLKNYVNDLVPEFLPAPEDVETGCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_6011 | H35F, K81D, | MRSSPGNMERIVICLMVIFLGTLVHKSSSQGQDREMIRMRQ | 1.059 | 1563 |
| | P107V, A112P | LIDIVDQLKNYVNDLVPEFLPAPEDVETNCEWSAFSCFQDA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_5959 | H35F, T110D, | MRSSPGNMERIVICLMVIFLGTLVHKSSSQGQDRFMIRMRQ | 1.059 | 1564 |
| | A112P, R115D | LIDIVDQLKNYVNDLVPEFLPAPEDVETNCEWSAFSCFQKA | | |
| | | QLKSANTGNNERIINVSIKKLKRKPPSDNPGRDQKHRLTCP | | |
| | | SCDSYEKKPPKEFLERFKSLLQKMIHQHLSSRTHGSEDS | | |
| IL-21_5010 | P107V, R114Q, | MRSSPGNMERIVICLMVIFLGTLVHKSSSQGQDRHMIRMRQ | 1.06 | 1565 |
| | R115D, T121E | LIDIVDQLKNYVNDLVPEFLPAPEDVETNCEWSAFSCFQKA | | |
| | | QLKSANTGNNERIINVSIKKLKRKVPSTNAGQDQKHRLECP | | |
| | | SCDSYEKKPPKEFLERFKSLLQKMIHQHLSSRTHGSEDS | | |
| IL-21_6076 | H35F, I45T, | MRSSPGNMERIVICLMVIFLGTLVHKSSSQGQDREMIRMRQ | 1.06 | 1566 |
| | V98E, R114Q | LIDTVDQLKNYVNDLVPEFLPAPEDVETNCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_5820 | V98E, R114Q | MRSSPGNMERIVICLMVIFLGTLVHKSSSQGQDRHMIRMRQ | 1.061 | 1567 |
| | | LIDIVDQLKNYVNDLVPEFLPAPEDVETNCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_5951 | H35F, A112P, | MRSSPGNMERIVICLMVIFLGTLVHKSSSQGQDREMIRMRQ | 1.062 | 1568 |
| | R114G, R115D | LIDIVDQLKNYVNDLVPEFLPAPEDVETNCEWSAFSCFQKA | | |
| | | QLKSANTGNNERIINVSIKKLKRKPPSTNPGGDQKHRLTCP | | |
| | | SCDSYEKKPPKEFLERFKSLLQKMIHQHLSSRTHGSEDS | | |
| IL-21_5226 | K81D, T110D, | MRSSPGNMERIVICLMVIFLGTLVHKSSSQGQDRHMIRMRQ | 1.062 | 1569 |
| | A112P, R119T | LIDIVDQLKNYVNDLVPEFLPAPEDVETNCEWSAFSCFQDA | | |
| | | QLKSANTGNNERIINVSIKKLKRKPPSDNPGRRQKHTLTCP | | |
| | | SCDSYEKKPPKEFLERFKSLLQKMIHQHLSSRTHGSEDS | | |
| IL-21_6029 | H35F, N70G, | MRSSPGNMERIVICLMVIFLGTLVHKSSSQGQDRFMIRMRQ | 1.062 | 1570 |
| | T110D, R115D | LIDIVDQLKNYVNDLVPEFLPAPEDVETGCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6366 | H35I, K81D, | MRSSPGNMERIVICLMVIFLGTLVHKSSSQGQDRIMIRMRQ | 1.063 | 1571 |
| | R114G | LIDIVDQLKNYVNDLVPEFLPAPEDVETNCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5881 | I45T, V98E, | MRSSPGNMERIVICLMVIFLGTLVHKSSSQGQDRHMIRMRQ | 1.063 | 1572 |
| | T110D, R114G | LIDTVDQLKNYVNDLVPEFLPAPEDVETNCEWSAFSCFQKA | | |
| | | QLKSANTGNNERIINESIKKLKRKPPSDNAGGRQKHRLTCP | | |
| | | SCDSYEKKPPKEFLERFKSLLQKMIHQHLSSRTHGSEDS | | |
| IL-21_6151 | H35I, K81D, | MRSSPGNMERIVICLMVIFLGTLVHKSSSQGQDRIMIRMRQ | 1.064 | 1573 |
| | T110D, R114G | LIDIVDQLKNYVNDLVPEFLPAPEDVETNCEWSAFSCFQDA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5038 | P107V, T110D, | MRSSPGNMERIVICLMVIFLGTLVHKSSSQGQDRHMIRMRQ | 1.064 | 1574 |
| | R115D, T121E | LIDIVDQLKNYVNDLVPEFLPAPEDVETNCEWSAFSCFQKA | | |
| | | QLKSANTGNNERIINVSIKKLKRKVPSDNAGRDQKHRLECP | | |
| | | SCDSYEKKPPKEFLERFKSLLQKMIHQHLSSRTHGSEDS | | |
| IL-21_5321 | N70G, R114G, | MRSSPGNMERIVICLMVIFLGTLVHKSSSQGQDRHMIRMRQ | 1.064 | 1575 |
| | R115G, H118D | LIDIVDQLKNYVNDLVPEFLPAPEDVETGCEWSAFSCFQKA | | |
| | | QLKSANTGNNERIINVSIKKLKRKPPSTNAGGGQKDRLTCP | | |
| | | SCDSYEKKPPKEFLERFKSLLQKMIHQHLSSRTHGSEDS | | |
| IL-21_5477 | N70G, K81D, | MRSSPGNMERIVICLMVIFLGTLVHKSSSQGQDRHMIRMRQ | 1.065 | 1576 |
| | A112P, H118D | LIDIVDQLKNYVNDLVPEFLPAPEDVETGCEWSAFSCFQDA | | |
| | | QLKSANTGNNERIINVSIKKLKRKPPSTNPGRRQKDRLTCP | | |
| | | SCDSYEKKPPKEFLERFKSLLQKMIHQHLSSRTHGSEDS | | |
| IL-21_5897 | I45T, K81D, | MRSSPGNMERIVICLMVIFLGTLVHKSSSQGQDRHMIRMRQ | 1.066 | 1577 |
| | A112P, R114Q | LIDTVDQLKNYVNDLVPEFLPAPEDVETNCEWSAFSCFQDA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5970 | H35F, P107V, | MRSSPGNMERIVICLMVIFLGTLVHKSSSQGQDREMIRMRQ | 1.066 | 1578 |
| | T110D, R115G | LIDIVDQLKNYVNDLVPEFLPAPEDVETNCEWSAFSCFQKA | | |
| | | QLKSANTGNNERIINVSIKKLKRKVPSDNAGRGQKHRLTCP | | |
| | | SCDSYEKKPPKEFLERFKSLLQKMIHQHLSSRTHGSEDS | | |
| IL-21_6168 | H35I, N70G, | MRSSPGNMERIVICLMVIFLGTLVHKSSSQGQDRIMIRMRQ | 1.066 | 1579 |
| | R114G, R115D | LIDIVDQLKNYVNDLVPEFLPAPEDVETGCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5887 | I45T, V98E, | MRSSPGNMERIVICLMVIFLGTLVHKSSSQGQDRHMIRMRQ | 1.067 | 1580 |
| | P107V, R114Q | LIDTVDQLKNYVNDLVPEFLPAPEDVETNCEWSAFSCFQKA | | |
| | | QLKSANTGNNERIINESIKKLKRKVPSTNAGQRQKHRLTCP | | |
| | | SCDSYEKKPPKEFLERFKSLLQKMIHQHLSSRTHGSEDS | | |
| IL-21_5757 | N70G, V98E, | MRSSPGNMERIVICLMVIFLGTLVHKSSSQGQDRHMIRMRQ | 1.069 | 1581 |
| | R114Q | LIDIVDQLKNYVNDLVPEFLPAPEDVETGCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_5882 | I45T, V98E, | MRSSPGNMERIVICLMVIFLGTLVHKSSSQGQDRHMIRMRQ | 1.069 | 1582 |
| | T110D, R114Q | LIDTVDQLKNYVNDLVPEFLPAPEDVETNCEWSAFSCFQKA | | |
| | | QLKSANTGNNERIINESIKKLKRKPPSDNAGQRQKHRLTCP | | |
| | | SCDSYEKKPPKEFLERFKSLLQKMIHQHLSSRTHGSEDS | | |
| IL-21_6315 | H35F, K81D, | MRSSPGNMERIVICLMVIFLGTLVHKSSSQGQDREMIRMRQ | 1.071 | 1583 |
| | A112P | LIDIVDQLKNYVNDLVPEFLPAPEDVETNCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5647 | V98E, P107V, | MRSSPGNMERIVICLMVIFLGTLVHKSSSQGQDRHMIRMRQ | 1.072 | 1584 |
| | R119T | LIDIVDQLKNYVNDLVPEFLPAPEDVETNCEWSAFSCFQKA | | |
| | | QLKSANTGNNERIINESIKKLKRKVPSTNAGRRQKHTLTCP | | |
| | | SCDSYEKKPPKEFLERFKSLLQKMIHQHLSSRTHGSEDS | | |
| IL-21_5187 | K81D, A112P, | MRSSPGNMERIVICLMVIFLGTLVHKSSSQGQDRHMIRMRQ | 1.073 | 1585 |
| | R119T, T121E | LIDIVDQLKNYVNDLVPEFLPAPEDVETNCEWSAFSCFQDA | | |
| | | QLKSANTGNNERIINVSIKKLKRKPPSTNPGRRQKHTLECP | | |
| | | SCDSYEKKPPKEFLERFKSLLQKMIHQHLSSRTHGSEDS | | |
| IL-21_5879 | I45T, V98E, | MRSSPGNMERIVICLMVIFLGTLVHKSSSQGQDRHMIRMRQ | 1.073 | 1586 |
| | T110D, R115G | LIDTVDQLKNYVNDLVPEFLPAPEDVETNCEWSAFSCFQKA | | |
| | | QLKSANTGNNERIINESIKKLKRKPPSDNAGRGQKHRLTCP | | |
| | | SCDSYEKKPPKEFLERFKSLLQKMIHQHLSSRTHGSEDS | | |
| IL-21_5890 | I45T, K81D, | MRSSPGNMERIVICLMVIFLGTLVHKSSSQGQDRHMIRMRQ | 1.073 | 1587 |
| | R114G, R115G | LIDTVDQLKNYVNDLVPEFLPAPEDVETNCEWSAFSCFQDA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_6288 | H35F, R114Q, | MRSSPGNMERIVICLMVIFLGTLVHKSSSQGQDRFMIRMRQ | 1.073 | 1588 |
| | R115D | LIDIVDQLKNYVNDLVPEFLPAPEDVETNCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5681 | K81D, T110D, | MRSSPGNMERIVICLMVIFLGTLVHKSSSQGQDRHMIRMRQ | 1.074 | 1589 |
| | T121E | LIDIVDQLKNYVNDLVPEFLPAPEDVETNCEWSAFSCFQDA | | |
| | | QLKSANTGNNERIINVSIKKLKRKPPSDNAGRRQKHRLECP | | |
| | | SCDSYEKKPPKEFLERFKSLLQKMIHQHLSSRTHGSEDS | | |
| IL-21_5947 | I45T, N70G, | MRSSPGNMERIVICLMVIFLGTLVHKSSSQGQDRHMIRMRQ | 1.075 | 1590 |
| | K81D, T110D | LIDTVDQLKNYVNDLVPEFLPAPEDVETGCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_4952 | T110D, R114G, | MRSSPGNMERIVICLMVIFLGTLVHKSSSQGQDRHMIRMRQ | 1.076 | 1591 |
| | H118D, T121E | LIDIVDQLKNYVNDLVPEFLPAPEDVETNCEWSAFSCFQKA | | |
| | | QLKSANTGNNERIINVSIKKLKRKPPSDNAGGRQKDRLECP | | |
| | | SCDSYEKKPPKEFLERFKSLLQKMIHQHLSSRTHGSEDS | | |
| IL-21_4911 | R114G, R115D, | MRSSPGNMERIVICLMVIFLGTLVHKSSSQGQDRHMIRMRQ | 1.076 | 1592 |
| | H118D, R119T | LIDIVDQLKNYVNDLVPEFLPAPEDVETNCEWSAFSCFQKA | | |
| | | QLKSANTGNNERIINVSIKKLKRKPPSTNAGGDQKDTLTCP | | |
| | | SCDSYEKKPPKEFLERFKSLLQKMIHQHLSSRTHGSEDS | | |
| IL-21_5634 | V98E, A112P, | MRSSPGNMERIVICLMVIFLGTLVHKSSSQGQDRHMIRMRQ | 1.077 | 1593 |
| | R115G | LIDIVDQLKNYVNDLVPEFLPAPEDVETNCEWSAFSCFQKA | | |
| | | QLKSANTGNNERIINESIKKLKRKPPSTNPGRGQKHRLTCP | | |
| | | SCDSYEKKPPKEFLERFKSLLQKMIHQHLSSRTHGSEDS | | |
| IL-21_6221 | H35I, I45T, | MRSSPGNMERIVICLMVIFLGTLVHKSSSQGQDRIMIRMRQ | 1.078 | 1594 |
| | V98E, R114G | LIDTVDQLKNYVNDLVPEFLPAPEDVETNCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5122 | V98E, T110D, | MRSSPGNMERIVICLMVIFLGTLVHKSSSQGQDRHMIRMRQ | 1.078 | 1595 |
| | A112P, R119T | LIDIVDQLKNYVNDLVPEFLPAPEDVETNCEWSAFSCFQKA | | |
| | | QLKSANTGNNERIINESIKKLKRKPPSDNPGRRQKHTLTCP | | |
| | | SCDSYEKKPPKEFLERFKSLLQKMIHQHLSSRTHGSEDS | | |
| IL-21_5621 | V98E, R114G, | MRSSPGNMERIVICLMVIFLGTLVHKSSSQGQDRHMIRMRQ | 1.08 | 1596 |
| | T121E | LIDIVDQLKNYVNDLVPEFLPAPEDVETNCEWSAFSCFQKA | | |
| | | QLKSANTGNNERIINESIKKLKRKPPSTNAGGRQKHRLECP | | |
| | | SCDSYEKKPPKEFLERFKSLLQKMIHQHLSSRTHGSEDS | | |
| IL-21_6057 | H35F, I45T, | MRSSPGNMERIVICLMVIFLGTLVHKSSSQGQDREMIRMRQ | 1.083 | 1597 |
| | R114Q, R115D | LIDTVDQLKNYVNDLVPEFLPAPEDVETNCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_6025 | H35F, N70G, | MRSSPGNMERIVICLMVIFLGTLVHKSSSQGQDRFMIRMRQ | 1.084 | 1598 |
| | A112P, R115D | LIDIVDQLKNYVNDLVPEFLPAPEDVETGCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5347 | N70G, A112P, | MRSSPGNMERIVICLMVIFLGTLVHKSSSQGQDRHMIRMRQ | 1.084 | 1599 |
| | R114G, R115D | LIDIVDQLKNYVNDLVPEFLPAPEDVETGCEWSAFSCFQKA | | |
| | | QLKSANTGNNERIINVSIKKLKRKPPSTNPGGDQKHRLTCP | | |
| | | SCDSYEKKPPKEFLERFKSLLQKMIHQHLSSRTHGSEDS | | |
| IL-21_4967 | T110D, R114Q, | MRSSPGNMERIVICLMVIFLGTLVHKSSSQGQDRHMIRMRQ | 1.084 | 1600 |
| | R115D, R119T | LIDIVDQLKNYVNDLVPEFLPAPEDVETNCEWSAFSCFQKA | | |
| | | QLKSANTGNNERIINVSIKKLKRKPPSDNAGQDQKHTLTCP | | |
| | | SCDSYEKKPPKEFLERFKSLLQKMIHQHLSSRTHGSEDS | | |
| IL-21_5867 | I45T, P107V, | MRSSPGNMERIVICLMVIFLGTLVHKSSSQGQDRHMIRMRQ | 1.084 | 1601 |
| | T110D, R115D | LIDTVDQLKNYVNDLVPEFLPAPEDVETNCEWSAFSCFQKA | | |
| | | QLKSANTGNNERIINVSIKKLKRKVPSDNAGRDQKHRLTCP | | |
| | | SCDSYEKKPPKEFLERFKSLLQKMIHQHLSSRTHGSEDS | | |
| IL-21_5885 | I45T, V98E, | MRSSPGNMERIVICLMVIFLGTLVHKSSSQGQDRHMIRMRQ | 1.084 | 1602 |
| | P107V, R115D | LIDTVDQLKNYVNDLVPEFLPAPEDVETNCEWSAFSCFQKA | | |
| | | QLKSANTGNNERIINESIKKLKRKVPSTNAGRDQKHRLTCP | | |
| | | SCDSYEKKPPKEFLERFKSLLQKMIHQHLSSRTHGSEDS | | |
| IL-21_5913 | I45T, K81D, | MRSSPGNMERIVICLMVIFLGTLVHKSSSQGQDRHMIRMRQ | 1.085 | 1603 |
| | V98E, A112P | LIDTVDQLKNYVNDLVPEFLPAPEDVETNCEWSAFSCFQDA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5355 | N70G, T110D, | MRSSPGNMERIVICLMVIFLGTLVHKSSSQGQDRHMIRMRQ | 1.085 | 1604 |
| | H118D, R119T | LIDIVDQLKNYVNDLVPEFLPAPEDVETGCEWSAFSCFQKA | | |
| | | QLKSANTGNNERIINVSIKKLKRKPPSDNAGRRQKDTLTCP | | |
| | | SCDSYEKKPPKEFLERFKSLLQKMIHQHLSSRTHGSEDS | | |
| IL-21_5987 | H35F, V98E, | MRSSPGNMERIVICLMVIFLGTLVHKSSSQGQDREMIRMRQ | 1.085 | 1605 |
| | T110D, A112P | LIDIVDQLKNYVNDLVPEFLPAPEDVETNCEWSAFSCFQKA | | |
| | | QLKSANTGNNERIINESIKKLKRKPPSDNPGRRQKHRLTCP | | |
| | | SCDSYEKKPPKEFLERFKSLLQKMIHQHLSSRTHGSEDS | | |
| IL-21_5391 | N70G, P107V, | MRSSPGNMERIVICLMVIFLGTLVHKSSSQGQDRHMIRMRQ | 1.087 | 1606 |
| | R114G, R115G | LIDIVDQLKNYVNDLVPEFLPAPEDVETGCEWSAFSCFQKA | | |
| | | QLKSANTGNNERIINVSIKKLKRKVPSTNAGGGQKHRLTCP | | |
| | | SCDSYEKKPPKEFLERFKSLLQKMIHQHLSSRTHGSEDS | | |
| IL-21_5901 | I45T, K81D, | MRSSPGNMERIVICLMVIFLGTLVHKSSSQGQDRHMIRMRQ | 1.088 | 1607 |
| | T110D, R114Q | LIDTVDQLKNYVNDLVPEFLPAPEDVETNCEWSAFSCFQDA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5721 | N70G, R114G, | MRSSPGNMERIVICLMVIFLGTLVHKSSSQGQDRHMIRMRQ | 1.089 | 1608 |
| | R115D | LIDIVDQLKNYVNDLVPEFLPAPEDVETGCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_6022 | H35F, N70G, | MRSSPGNMERIVICLMVIFLGTLVHKSSSQGQDREMIRMRQ | 1.089 | 1609 |
| | R114Q, R115G | LIDIVDQLKNYVNDLVPEFLPAPEDVETGCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5636 | V98E, A112P, | MRSSPGNMERIVICLMVIFLGTLVHKSSSQGQDRHMIRMRQ | 1.089 | 1610 |
| | R114G | LIDIVDQLKNYVNDLVPEFLPAPEDVETNCEWSAFSCFQKA | | |
| | | QLKSANTGNNERIINESIKKLKRKPPSTNPGGRQKHRLTCP | | |
| | | SCDSYEKKPPKEFLERFKSLLQKMIHQHLSSRTHGSEDS | | |
| IL-21_5896 | I45T, K81D, | MRSSPGNMERIVICLMVIFLGTLVHKSSSQGQDRHMIRMRQ | 1.09 | 1611 |
| | A112P, R114G | LIDTVDQLKNYVNDLVPEFLPAPEDVETNCEWSAFSCFQDA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5011 | P107V, R114Q, | MRSSPGNMERIVICLMVIFLGTLVHKSSSQGQDRHMIRMRQ | 1.093 | 1612 |
| | R115D, R119T | LIDIVDQLKNYVNDLVPEFLPAPEDVETNCEWSAFSCFQKA | | |
| | | QLKSANTGNNERIINVSIKKLKRKVPSTNAGQDQKHTLTCP | | |
| | | SCDSYEKKPPKEFLERFKSLLQKMIHQHLSSRTHGSEDS | | |
| IL-21_5654 | V98E, P107V, | MRSSPGNMERIVICLMVIFLGTLVHKSSSQGQDRHMIRMRQ | 1.094 | 1613 |
| | T110D | LIDIVDQLKNYVNDLVPEFLPAPEDVETNCEWSAFSCFQKA | | |
| | | QLKSANTGNNERIINESIKKLKRKVPSDNAGRRQKHRLTCP | | |
| | | SCDSYEKKPPKEFLERFKSLLQKMIHQHLSSRTHGSEDS | | |
| IL-21_4905 | R114G, H118D, | MRSSPGNMERIVICLMVIFLGTLVHKSSSQGQDRHMIRMRQ | 1.094 | 1614 |
| | R119T, T121E | LIDIVDQLKNYVNDLVPEFLPAPEDVETNCEWSAFSCFQKA | | |
| | | QLKSANTGNNERIINVSIKKLKRKPPSTNAGGRQKDTLECP | | |
| | | SCDSYEKKPPKEFLERFKSLLQKMIHQHLSSRTHGSEDS | | |
| IL-21_5009 | P107V, R114Q, | MRSSPGNMERIVICLMVIFLGTLVHKSSSQGQDRHMIRMRQ | 1.097 | 1615 |
| | R115G, H118D | LIDIVDQLKNYVNDLVPEFLPAPEDVETNCEWSAFSCFQKA | | |
| | | QLKSANTGNNERIINVSIKKLKRKVPSTNAGQGQKDRLTCP | | |
| | | SCDSYEKKPPKEFLERFKSLLQKMIHQHLSSRTHGSEDS | | |
| IL-21_6043 | H35F, N70G, | MRSSPGNMERIVICLMVIFLGTLVHKSSSQGQDRFMIRMRQ | 1.097 | 1616 |
| | V98E, A112P | LIDIVDQLKNYVNDLVPEFLPAPEDVETGCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5670 | K81D, R114Q, | MRSSPGNMERIVICLMVIFLGTLVHKSSSQGQDRHMIRMRQ | 1.099 | 1617 |
| | R119T | LIDIVDQLKNYVNDLVPEFLPAPEDVETNCEWSAFSCFQDA | | |
| | | QLKSANTGNNERIINVSIKKLKRKPPSTNAGQRQKHTLTCP | | |
| | | SCDSYEKKPPKEFLERFKSLLQKMIHQHLSSRTHGSEDS | | |
| IL-21_5819 | V98E, R114G | MRSSPGNMERIVICLMVIFLGTLVHKSSSQGQDRHMIRMRQ | 1.1 | 1618 |
| | | LIDIVDQLKNYVNDLVPEFLPAPEDVETNCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5972 | H35F, P107V, | MRSSPGNMERIVICLMVIFLGTLVHKSSSQGQDRFMIRMRQ | 1.101 | 1619 |
| | T110D, R114G | LIDIVDQLKNYVNDLVPEFLPAPEDVETNCEWSAFSCFQKA | | |
| | | QLKSANTGNNERIINVSIKKLKRKVPSDNAGGRQKHRLTCP | | |
| | | SCDSYEKKPPKEFLERFKSLLQKMIHQHLSSRTHGSEDS | | |
| IL-21_4924 | A112P, R115D, | MRSSPGNMERIVICLMVIFLGTLVHKSSSQGQDRHMIRMRQ | 1.102 | 1620 |
| | H118D, T121E | LIDIVDQLKNYVNDLVPEFLPAPEDVETNCEWSAFSCFQKA | | |
| | | QLKSANTGNNERIINVSIKKLKRKPPSTNPGRDQKDRLECP | | |
| | | SCDSYEKKPPKEFLERFKSLLQKMIHQHLSSRTHGSEDS | | |
| IL-21_4977 | T110D, A112P, | MRSSPGNMERIVICLMVIFLGTLVHKSSSQGQDRHMIRMRQ | 1.102 | 1621 |
| | R115D, H118D | LIDIVDQLKNYVNDLVPEFLPAPEDVETNCEWSAFSCFQKA | | |
| | | QLKSANTGNNERIINVSIKKLKRKPPSDNPGRDQKDRLTCP | | |
| | | SCDSYEKKPPKEFLERFKSLLQKMIHQHLSSRTHGSEDS | | |
| IL-21_5876 | I45T, V98E, | MRSSPGNMERIVICLMVIFLGTLVHKSSSQGQDRHMIRMRQ | 1.103 | 1622 |
| | A112P, R115D | LIDTVDQLKNYVNDLVPEFLPAPEDVETNCEWSAFSCFQKA | | |
| | | QLKSANTGNNERIINESIKKLKRKPPSTNPGRDQKHRLTCP | | |
| | | SCDSYEKKPPKEFLERFKSLLQKMIHQHLSSRTHGSEDS | | |
| IL-21_5044 | P107V, T110D, | MRSSPGNMERIVICLMVIFLGTLVHKSSSQGQDRHMIRMRQ | 1.103 | 1623 |
| | R114G, R115G | LIDIVDQLKNYVNDLVPEFLPAPEDVETNCEWSAFSCFQKA | | |
| | | QLKSANTGNNERIINVSIKKLKRKVPSDNAGGGQKHRLTCP | | |
| | | SCDSYEKKPPKEFLERFKSLLQKMIHQHLSSRTHGSEDS | | |
| IL-21_6419 | H35I, V98E | MRSSPGNMERIVICLMVIFLGTLVHKSSSQGQDRIMIRMRQ | 1.104 | 1624 |
| | | LIDIVDQLKNYVNDLVPEFLPAPEDVETNCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_6360 | H35I, V98E, | MRSSPGNMERIVICLMVIFLGTLVHKSSSQGQDRIMIRMRQ | 1.105 | 1625 |
| | R114Q | LIDIVDQLKNYVNDLVPEFLPAPEDVETNCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_5873 | I45T, V98E, | MRSSPGNMERIVICLMVIFLGTLVHKSSSQGQDRHMIRMRQ | 1.105 | 1626 |
| | 1R14Q, R115G | LIDTVDQLKNYVNDLVPEFLPAPEDVETNCEWSAFSCFQKA | | |
| | | QLKSANTGNNERIINESIKKLKRKPPSTNAGQGQKHRLTCP | | |
| | | SCDSYEKKPPKEFLERFKSLLQKMIHQHLSSRTHGSEDS | | |
| IL-21_5751 | N70G, V98E, | MRSSPGNMERIVICLMVIFLGTLVHKSSSQGQDRHMIRMRQ | 1.105 | 1627 |
| | T121E | LIDIVDQLKNYVNDLVPEFLPAPEDVETGCEWSAFSCFQKA | | |
| | | QLKSANTGNNERIINESIKKLKRKPPSTNAGRRQKHRLECP | | |
| | | SCDSYEKKPPKEFLERFKSLLQKMIHQHLSSRTHGSEDS | | |
| IL-21_5314 | N70G, R115D, | MRSSPGNMERIVICLMVIFLGTLVHKSSSQGQDRHMIRMRQ | 1.107 | 1628 |
| | H118D, T121E | LIDIVDQLKNYVNDLVPEFLPAPEDVETGCEWSAFSCFQKA | | |
| | | QLKSANTGNNERIINVSIKKLKRKPPSTNAGRDQKDRLECP | | |
| | | SCDSYEKKPPKEFLERFKSLLQKMIHQHLSSRTHGSEDS | | |
| IL-21_6045 | H35F, N70G, | MRSSPGNMERIVICLMVIFLGTLVHKSSSQGQDREMIRMRQ | 1.109 | 1629 |
| | V98E, P107V | LIDIVDQLKNYVNDLVPEFLPAPEDVETGCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5161 | V98E, P107V, | MRSSPGNMERIVICLMVIFLGTLVHKSSSQGQDRHMIRMRQ | 1.11 | 1630 |
| | T110D, A112P | LIDIVDQLKNYVNDLVPEFLPAPEDVETNCEWSAFSCFQKA | | |
| | | QLKSANTGNNERIINESIKKLKRKVPSDNPGRRQKHRLTCP | | |
| | | SCDSYEKKPPKEFLERFKSLLQKMIHQHLSSRTHGSEDS | | |
| IL-21_5497 | N70G, K81D, | MRSSPGNMERIVICLMVIFLGTLVHKSSSQGQDRHMIRMRQ | 1.11 | 1631 |
| | P107V, A112P | LIDIVDQLKNYVNDLVPEFLPAPEDVETGCEWSAFSCFQDA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_5330 | N70G, R114Q, | MRSSPGNMERIVICLMVIFLGTLVHKSSSQGQDRHMIRMRQ | 1.11 | 1632 |
| | R115G, H118D | LIDIVDQLKNYVNDLVPEFLPAPEDVETGCEWSAFSCFQKA | | |
| | | QLKSANTGNNERIINVSIKKLKRKPPSTNAGQGQKDRLTCP | | |
| | | SCDSYEKKPPKEFLERFKSLLQKMIHQHLSSRTHGSEDS | | |
| IL-21_4909 | R114G, R115D, | MRSSPGNMERIVICLMVIFLGTLVHKSSSQGQDRHMIRMRQ | 1.11 | 1633 |
| | R119T, T121E | LIDIVDQLKNYVNDLVPEFLPAPEDVETNCEWSAFSCFQKA | | |
| | | QLKSANTGNNERIINVSIKKLKRKPPSTNAGGDQKHTLECP | | |
| | | SCDSYEKKPPKEFLERFKSLLQKMIHQHLSSRTHGSEDS | | |
| IL-21_5369 | N70G, T110D, | MRSSPGNMERIVICLMVIFLGTLVHKSSSQGQDRHMIRMRQ | 1.111 | 1634 |
| | R114Q, H118D | LIDIVDQLKNYVNDLVPEFLPAPEDVETGCEWSAFSCFQKA | | |
| | | QLKSANTGNNERIINVSIKKLKRKPPSDNAGQRQKDRLTCP | | |
| | | SCDSYEKKPPKEFLERFKSLLQKMIHQHLSSRTHGSEDS | | |
| IL-21_5817 | V98E, R115G | MRSSPGNMERIVICLMVIFLGTLVHKSSSQGQDRHMIRMRQ | 1.112 | 1635 |
| | | LIDIVDQLKNYVNDLVPEFLPAPEDVETNCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_5571 | T110D, R114Q, | MRSSPGNMERIVICLMVIFLGTLVHKSSSQGQDRHMIRMRQ | 1.113 | 1636 |
| | R115D | LIDIVDQLKNYVNDLVPEFLPAPEDVETNCEWSAFSCFQKA | | |
| | | QLKSANTGNNERIINVSIKKLKRKPPSDNAGQDQKHRLTCP | | |
| | | SCDSYEKKPPKEFLERFKSLLQKMIHQHLSSRTHGSEDS | | |
| IL-21_5086 | V98E, A112P, | MRSSPGNMERIVICLMVIFLGTLVHKSSSQGQDRHMIRMRQ | 1.113 | 1637 |
| | R115G, T121E | LIDIVDQLKNYVNDLVPEFLPAPEDVETNCEWSAFSCFQKA | | |
| | | QLKSANTGNNERIINESIKKLKRKPPSTNPGRGQKHRLECP | | |
| | | SCDSYEKKPPKEFLERFKSLLQKMIHQHLSSRTHGSEDS | | |
| IL-21_6063 | H35F, I45T, | MRSSPGNMERIVICLMVIFLGTLVHKSSSQGQDREMIRMRQ | 1.114 | 1638 |
| | T110D, R115D | LIDTVDQLKNYVNDLVPEFLPAPEDVETNCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6285 | H35F, R114G, | MRSSPGNMERIVICLMVIFLGTLVHKSSSQGQDREMIRMRQ | 1.114 | 1639 |
| | R115G | LIDIVDQLKNYVNDLVPEFLPAPEDVETNCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_6310 | H35F, V98E, | MRSSPGNMERIVICLMVIFLGTLVHKSSSQGQDREMIRMRQ | 1.115 | 1640 |
| | P107V | LIDIVDQLKNYVNDLVPEFLPAPEDVETNCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_4965 | T110D, R114Q, | MRSSPGNMERIVICLMVIFLGTLVHKSSSQGQDRHMIRMRQ | 1.116 | 1641 |
| | R115G, H118D | LIDIVDQLKNYVNDLVPEFLPAPEDVETNCEWSAFSCFQKA | | |
| | | QLKSANTGNNERIINVSIKKLKRKPPSDNAGQGQKDRLTCP | | |
| | | SCDSYEKKPPKEFLERFKSLLQKMIHQHLSSRTHGSEDS | | |
| IL-21_5142 | V98E, P107V, | MRSSPGNMERIVICLMVIFLGTLVHKSSSQGQDRHMIRMRQ | 1.117 | 1642 |
| | R114Q, T121E | LIDIVDQLKNYVNDLVPEFLPAPEDVETNCEWSAFSCFQKA | | |
| | | QLKSANTGNNERIINESIKKLKRKVPSTNAGQRQKHRLECP | | |
| | | SCDSYEKKPPKEFLERFKSLLQKMIHQHLSSRTHGSEDS | | |
| IL-21_6109 | H35I, P107V, | MRSSPGNMERIVICLMVIFLGTLVHKSSSQGQDRIMIRMRQ | 1.119 | 1643 |
| | R114G, R115G | LIDIVDQLKNYVNDLVPEFLPAPEDVETNCEWSAFSCFQKA | | |
| | | QLKSANTGNNERIINVSIKKLKRKVPSTNAGGGQKHRLTCP | | |
| | | SCDSYEKKPPKEFLERFKSLLQKMIHQHLSSRTHGSEDS | | |
| IL-21_5762 | N70G, K81D, | MRSSPGNMERIVICLMVIFLGTLVHKSSSQGQDRHMIRMRQ | 1.121 | 1644 |
| | R119T | LIDIVDQLKNYVNDLVPEFLPAPEDVETGCEWSAFSCFQDA | | |
| | | QLKSANTGNNERIINVSIKKLKRKPPSTNAGRRQKHTLTCP | | |
| | | SCDSYEKKPPKEFLERFKSLLQKMIHQHLSSRTHGSEDS | | |
| IL-21_5000 | P107V, R114G, | MRSSPGNMERIVICLMVIFLGTLVHKSSSQGQDRHMIRMRQ | 1.121 | 1645 |
| | R115G, H118D | LIDIVDQLKNYVNDLVPEFLPAPEDVETNCEWSAFSCFQKA | | |
| | | QLKSANTGNNERIINVSIKKLKRKVPSTNAGGGQKDRLTCP | | |
| | | SCDSYEKKPPKEFLERFKSLLQKMIHQHLSSRTHGSEDS | | |
| IL-21_5937 | I45T, N70G, | MRSSPGNMERIVICLMVIFLGTLVHKSSSQGQDRHMIRMRQ | 1.122 | 1646 |
| | V98E, R114G | LIDTVDQLKNYVNDLVPEFLPAPEDVETGCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_4914 | R114Q, R115G, | MRSSPGNMERIVICLMVIFLGTLVHKSSSQGQDRHMIRMRQ | 1.123 | 1647 |
| | H118D, T121E | LIDIVDQLKNYVNDLVPEFLPAPEDVETNCEWSAFSCFQKA | | |
| | | QLKSANTGNNERIINVSIKKLKRKPPSTNAGQGQKDRLECP | | |
| | | SCDSYEKKPPKEFLERFKSLLQKMIHQHLSSRTHGSEDS | | |
| IL-21_5318 | N70G, R114G, | MRSSPGNMERIVICLMVIFLGTLVHKSSSQGQDRHMIRMRQ | 1.127 | 1648 |
| | H118D, R119T | LIDIVDQLKNYVNDLVPEFLPAPEDVETGCEWSAFSCFQKA | | |
| | | QLKSANTGNNERIINVSIKKLKRKPPSTNAGGRQKDTLTCP | | |
| | | SCDSYEKKPPKEFLERFKSLLQKMIHQHLSSRTHGSEDS | | |
| IL-21_6363 | H35I, V98E, | MRSSPGNMERIVICLMVIFLGTLVHKSSSQGQDRIMIRMRQ | 1.127 | 1649 |
| | P107V | LIDIVDQLKNYVNDLVPEFLPAPEDVETNCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_6369 | H35I, K81D, | MRSSPGNMERIVICLMVIFLGTLVHKSSSQGQDRIMIRMRQ | 1.129 | 1650 |
| | T110D | LIDIVDQLKNYVNDLVPEFLPAPEDVETNCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_4976 | T110D, A112P, | MRSSPGNMERIVICLMVIFLGTLVHKSSSQGQDRHMIRMRQ | 1.129 | 1651 |
| | R115D, R119T | LIDIVDQLKNYVNDLVPEFLPAPEDVETNCEWSAFSCFQKA | | |
| | | QLKSANTGNNERIINVSIKKLKRKPPSDNPGRDQKHTLTCP | | |
| | | SCDSYEKKPPKEFLERFKSLLQKMIHQHLSSRTHGSEDS | | |
| IL-21_6339 | H35I, R114G, | MRSSPGNMERIVICLMVIFLGTLVHKSSSQGQDRIMIRMRQ | 1.13 | 1652 |
| | R115D | LIDIVDQLKNYVNDLVPEFLPAPEDVETNCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5021 | P107V, A112P, | MRSSPGNMERIVICLMVIFLGTLVHKSSSQGQDRHMIRMRQ | 1.132 | 1653 |
| | R115D, H118D | LIDIVDQLKNYVNDLVPEFLPAPEDVETNCEWSAFSCFQKA | | |
| | | QLKSANTGNNERIINVSIKKLKRKVPSTNPGRDQKDRLTCP | | |
| | | SCDSYEKKPPKEFLERFKSLLQKMIHQHLSSRTHGSEDS | | |
| IL-21_5006 | P107V, R114Q, | MRSSPGNMERIVICLMVIFLGTLVHKSSSQGQDRHMIRMRQ | 1.132 | 1654 |
| | H118D, R119T | LIDIVDQLKNYVNDLVPEFLPAPEDVETNCEWSAFSCFQKA | | |
| | | QLKSANTGNNERIINVSIKKLKRKVPSTNAGQRQKDTLTCP | | |
| | | SCDSYEKKPPKEFLERFKSLLQKMIHQHLSSRTHGSEDS | | |
| IL-21_5048 | P107V, T110D, | MRSSPGNMERIVICLMVIFLGTLVHKSSSQGQDRHMIRMRQ | 1.133 | 1655 |
| | R114Q, H118D | LIDIVDQLKNYVNDLVPEFLPAPEDVETNCEWSAFSCFQKA | | |
| | | QLKSANTGNNERIINVSIKKLKRKVPSDNAGQRQKDRLTCP | | |
| | | SCDSYEKKPPKEFLERFKSLLQKMIHQHLSSRTHGSEDS | | |
| IL-21_5042 | P107V, T110D, | MRSSPGNMERIVICLMVIFLGTLVHKSSSQGQDRHMIRMRQ | 1.135 | 1656 |
| | R114G, R119T | LIDIVDQLKNYVNDLVPEFLPAPEDVETNCEWSAFSCFQKA | | |
| | | QLKSANTGNNERIINVSIKKLKRKVPSDNAGGRQKHTLTCP | | |
| | | SCDSYEKKPPKEFLERFKSLLQKMIHQHLSSRTHGSEDS | | |
| IL-21_4925 | A112P, R115D, | MRSSPGNMERIVICLMVIFLGTLVHKSSSQGQDRHMIRMRQ | 1.136 | 1657 |
| | H118D, R119T | LIDIVDQLKNYVNDLVPEFLPAPEDVETNCEWSAFSCFQKA | | |
| | | QLKSANTGNNERIINVSIKKLKRKPPSTNPGRDQKDTLTCP | | |
| | | SCDSYEKKPPKEFLERFKSLLQKMIHQHLSSRTHGSEDS | | |
| IL-21_5940 | I45T, N70G, | MRSSPGNMERIVICLMVIFLGTLVHKSSSQGQDRHMIRMRQ | 1.136 | 1658 |
| | V98E, T110D | LIDTVDQLKNYVNDLVPEFLPAPEDVETGCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5043 | P107V, T110D, | MRSSPGNMERIVICLMVIFLGTLVHKSSSQGQDRHMIRMRQ | 1.139 | 1659 |
| | R114G, H118D | LIDIVDQLKNYVNDLVPEFLPAPEDVETNCEWSAFSCFQKA | | |
| | | QLKSANTGNNERIINVSIKKLKRKVPSDNAGGRQKDRLTCP | | |
| | | SCDSYEKKPPKEFLERFKSLLQKMIHQHLSSRTHGSEDS | | |
| IL-21_5188 | K81D, A112P, | MRSSPGNMERIVICLMVIFLGTLVHKSSSQGQDRHMIRMRQ | 1.139 | 1660 |
| | H118D, T121E | LIDIVDQLKNYVNDLVPEFLPAPEDVETNCEWSAFSCFQDA | | |
| | | QLKSANTGNNERIINVSIKKLKRKPPSTNPGRRQKDRLECP | | |
| | | SCDSYEKKPPKEFLERFKSLLQKMIHQHLSSRTHGSEDS | | |
| IL-21_5386 | N70G, P107V, | MRSSPGNMERIVICLMVIFLGTLVHKSSSQGQDRHMIRMRQ | 1.143 | 1661 |
| | R115D, R119T | LIDIVDQLKNYVNDLVPEFLPAPEDVETGCEWSAFSCFQKA | | |
| | | QLKSANTGNNERIINVSIKKLKRKVPSTNAGRDQKHTLTCP | | |
| | | SCDSYEKKPPKEFLERFKSLLQKMIHQHLSSRTHGSEDS | | |
| IL-21_6167 | H35I, N70G, | MRSSPGNMERIVICLMVIFLGTLVHKSSSQGQDRIMIRMRQ | 1.143 | 1662 |
| | R114G, R115G | LIDIVDQLKNYVNDLVPEFLPAPEDVETGCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5894 | I45T, K81D, | MRSSPGNMERIVICLMVIFLGTLVHKSSSQGQDRHMIRMRQ | 1.144 | 1663 |
| | A112P, R115G | LIDTVDQLKNYVNDLVPEFLPAPEDVETNCEWSAFSCFQDA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5121 | V98E, T110D, | MRSSPGNMERIVICLMVIFLGTLVHKSSSQGQDRHMIRMRQ | 1.144 | 1664 |
| | A112P, T121E | LIDIVDQLKNYVNDLVPEFLPAPEDVETNCEWSAFSCFQKA | | |
| | | QLKSANTGNNERIINESIKKLKRKPPSDNPGRRQKHRLECP | | |
| | | SCDSYEKKPPKEFLERFKSLLQKMIHQHLSSRTHGSEDS | | |
| IL-21_5147 | V98E, P107V, | MRSSPGNMERIVICLMVIFLGTLVHKSSSQGQDRHMIRMRQ | 1.144 | 1665 |
| | A112P, T121E | LIDIVDQLKNYVNDLVPEFLPAPEDVETNCEWSAFSCFQKA | | |
| | | QLKSANTGNNERIINESIKKLKRKVPSTNPGRRQKHRLECP | | |
| | | SCDSYEKKPPKEFLERFKSLLQKMIHQHLSSRTHGSEDS | | |
| IL-21_5083 | V98E, A112P, | MRSSPGNMERIVICLMVIFLGTLVHKSSSQGQDRHMIRMRQ | 1.146 | 1666 |
| | R119T, T121E | LIDIVDQLKNYVNDLVPEFLPAPEDVETNCEWSAFSCFQKA | | |
| | | QLKSANTGNNERIINESIKKLKRKPPSTNPGRRQKHTLECP | | |
| | | SCDSYEKKPPKEFLERFKSLLQKMIHQHLSSRTHGSEDS | | |
| IL-21_5396 | N70G, P107V, | MRSSPGNMERIVICLMVIFLGTLVHKSSSQGQDRHMIRMRQ | 1.146 | 1667 |
| | R114Q, R115G | LIDIVDQLKNYVNDLVPEFLPAPEDVETGCEWSAFSCFQKA | | |
| | | QLKSANTGNNERIINVSIKKLKRKVPSTNAGQGQKHRLTCP | | |
| | | SCDSYEKKPPKEFLERFKSLLQKMIHQHLSSRTHGSEDS | | |
| IL-21_5822 | V98E, T110D | MRSSPGNMERIVICLMVIFLGTLVHKSSSQGQDRHMIRMRQ | 1.148 | 1668 |
| | | LIDIVDQLKNYVNDLVPEFLPAPEDVETNCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5309 | N70G, H118D, | MRSSPGNMERIVICLMVIFLGTLVHKSSSQGQDRHMIRMRQ | 1.148 | 1669 |
| | R119T, T121E | LIDIVDQLKNYVNDLVPEFLPAPEDVETGCEWSAFSCFQKA | | |
| | | QLKSANTGNNERIINVSIKKLKRKPPSTNAGRRQKDTLECP | | |
| | | SCDSYEKKPPKEFLERFKSLLQKMIHQHLSSRTHGSEDS | | |
| IL-21_6073 | H35F, I45T, | MRSSPGNMERIVICLMVIFLGTLVHKSSSQGQDREMIRMRQ | 1.148 | 1670 |
| | V98E, R115G | LIDTVDQLKNYVNDLVPEFLPAPEDVETNCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_4982 | T110D, A112P, | MRSSPGNMERIVICLMVIFLGTLVHKSSSQGQDRHMIRMRQ | 1.148 | 1671 |
| | R114G, R115D | LIDIVDQLKNYVNDLVPEFLPAPEDVETNCEWSAFSCFQKA | | |
| | | QLKSANTGNNERIINVSIKKLKRKPPSDNPGGDQKHRLTCP | | |
| | | SCDSYEKKPPKEFLERFKSLLQKMIHQHLSSRTHGSEDS | | |
| IL-21_5331 | N70G, R114Q, | MRSSPGNMERIVICLMVIFLGTLVHKSSSQGQDRHMIRMRQ | 1.149 | 1672 |
| | R115D, T121E | LIDIVDQLKNYVNDLVPEFLPAPEDVETGCEWSAFSCFQKA | | |
| | | QLKSANTGNNERIINVSIKKLKRKPPSTNAGQDQKHRLECP | | |
| | | SCDSYEKKPPKEFLERFKSLLQKMIHQHLSSRTHGSEDS | | |
| IL-21_5716 | N70G, R115D, | MRSSPGNMERIVICLMVIFLGTLVHKSSSQGQDRHMIRMRQ | 1.149 | 1673 |
| | H118D | LIDIVDQLKNYVNDLVPEFLPAPEDVETGCEWSAFSCFQKA | | |
| | | QLKSANTGNNERIINVSIKKLKRKPPSTNAGRDQKDRLTCP | | |
| | | SCDSYEKKPPKEFLERFKSLLQKMIHQHLSSRTHGSEDS | | |
| IL-21_6020 | H35F, N70G, | MRSSPGNMERIVICLMVIFLGTLVHKSSSQGQDRFMIRMRQ | 1.149 | 1674 |
| | R114G, R115G | LIDIVDQLKNYVNDLVPEFLPAPEDVETGCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_6409 | H35F, K81D | MRSSPGNMERIVICLMVIFLGTLVHKSSSQGQDRFMIRMRQ | 1.15 | 1675 |
| | | LIDIVDQLKNYVNDLVPEFLPAPEDVETNCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5359 | N70G, T110D, | MRSSPGNMERIVICLMVIFLGTLVHKSSSQGQDRHMIRMRQ | 1.151 | 1676 |
| | R115D, T121E | LIDIVDQLKNYVNDLVPEFLPAPEDVETGCEWSAFSCFQKA | | |
| | | QLKSANTGNNERIINVSIKKLKRKPPSDNAGRDQKHRLECP | | |
| | | SCDSYEKKPPKEFLERFKSLLQKMIHQHLSSRTHGSEDS | | |
| IL-21_5674 | K81D, A112P, | MRSSPGNMERIVICLMVIFLGTLVHKSSSQGQDRHMIRMRQ | 1.151 | 1677 |
| | T121E | LIDIVDQLKNYVNDLVPEFLPAPEDVETNCEWSAFSCFQDA | | |
| | | QLKSANTGNNERIINVSIKKLKRKPPSTNPGRRQKHRLECP | | |
| | | SCDSYEKKPPKEFLERFKSLLQKMIHQHLSSRTHGSEDS | | |
| IL-21_5886 | I45T, V98E, | MRSSPGNMERIVICLMVIFLGTLVHKSSSQGQDRHMIRMRQ | 1.151 | 1678 |
| | P107V, R114G | LIDTVDQLKNYVNDLVPEFLPAPEDVETNCEWSAFSCFQKA | | |
| | | QLKSANTGNNERIINESIKKLKRKVPSTNAGGRQKHRLTCP | | |
| | | SCDSYEKKPPKEFLERFKSLLQKMIHQHLSSRTHGSEDS | | |
| IL-21_4953 | T110D, R114G, | MRSSPGNMERIVICLMVIFLGTLVHKSSSQGQDRHMIRMRQ | 1.152 | 1679 |
| | H118D, R119T | LIDIVDQLKNYVNDLVPEFLPAPEDVETNCEWSAFSCFQKA | | |
| | | QLKSANTGNNERIINVSIKKLKRKPPSDNAGGRQKDTLTCP | | |
| | | SCDSYEKKPPKEFLERFKSLLQKMIHQHLSSRTHGSEDS | | |
| IL-21_4988 | P107V, H118D, | MRSSPGNMERIVICLMVIFLGTLVHKSSSQGQDRHMIRMRQ | 1.152 | 1680 |
| | R119T, T121E | LIDIVDQLKNYVNDLVPEFLPAPEDVETNCEWSAFSCFQKA | | |
| | | QLKSANTGNNERIINVSIKKLKRKVPSTNAGRRQKDTLECP | | |
| | | SCDSYEKKPPKEFLERFKSLLQKMIHQHLSSRTHGSEDS | | |
| IL-21_6203 | H35I, I45T, | MRSSPGNMERIVICLMVIFLGTLVHKSSSQGQDRIMIRMRQ | 1.153 | 1681 |
| | R114Q, R115D | LIDTVDQLKNYVNDLVPEFLPAPEDVETNCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5446 | N70G, V98E, | MRSSPGNMERIVICLMVIFLGTLVHKSSSQGQDRHMIRMRQ | 1.154 | 1682 |
| | T110D, A112P | LIDIVDQLKNYVNDLVPEFLPAPEDVETGCEWSAFSCFQKA | | |
| | | QLKSANTGNNERIINESIKKLKRKPPSDNPGRRQKHRLTCP | | |
| | | SCDSYEKKPPKEFLERFKSLLQKMIHQHLSSRTHGSEDS | | |
| IL-21_6034 | H35F, N70G, | MRSSPGNMERIVICLMVIFLGTLVHKSSSQGQDRFMIRMRQ | 1.154 | 1683 |
| | P107V, R115D | LIDIVDQLKNYVNDLVPEFLPAPEDVETGCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5031 | P107V, A112P, | MRSSPGNMERIVICLMVIFLGTLVHKSSSQGQDRHMIRMRQ | 1.155 | 1684 |
| | R114Q, R115D | LIDIVDQLKNYVNDLVPEFLPAPEDVETNCEWSAFSCFQKA | | |
| | | QLKSANTGNNERIINVSIKKLKRKVPSTNPGQDQKHRLTCP | | |
| | | SCDSYEKKPPKEFLERFKSLLQKMIHQHLSSRTHGSEDS | | |
| IL-21_4946 | T110D, R115G, | MRSSPGNMERIVICLMVIFLGTLVHKSSSQGQDRHMIRMRQ | 1.156 | 1685 |
| | H118D, T121E | LIDIVDQLKNYVNDLVPEFLPAPEDVETNCEWSAFSCFQKA | | |
| | | QLKSANTGNNERIINVSIKKLKRKPPSDNAGRGQKDRLECP | | |
| | | SCDSYEKKPPKEFLERFKSLLQKMIHQHLSSRTHGSEDS | | |
| IL-21_6054 | H35F, I45T, | MRSSPGNMERIVICLMVIFLGTLVHKSSSQGQDRFMIRMRQ | 1.157 | 1686 |
| | R114G, R115G | LIDTVDQLKNYVNDLVPEFLPAPEDVETNCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5358 | N70G, T110D, | MRSSPGNMERIVICLMVIFLGTLVHKSSSQGQDRHMIRMRQ | 1.157 | 1687 |
| | R115G, H118D | LIDIVDQLKNYVNDLVPEFLPAPEDVETGCEWSAFSCFQKA | | |
| | | QLKSANTGNNERIINVSIKKLKRKPPSDNAGRGQKDRLTCP | | |
| | | SCDSYEKKPPKEFLERFKSLLQKMIHQHLSSRTHGSEDS | | |
| IL-21_5638 | V98E, T110D, | MRSSPGNMERIVICLMVIFLGTLVHKSSSQGQDRHMIRMRQ | 1.161 | 1688 |
| | T121E | LIDIVDQLKNYVNDLVPEFLPAPEDVETNCEWSAFSCFQKA | | |
| | | QLKSANTGNNERIINESIKKLKRKPPSDNAGRRQKHRLECP | | |
| | | SCDSYEKKPPKEFLERFKSLLQKMIHQHLSSRTHGSEDS | | |
| IL-21_5099 | V98E, A112P, | MRSSPGNMERIVICLMVIFLGTLVHKSSSQGQDRHMIRMRQ | 1.162 | 1689 |
| | R114Q, H118D | LIDIVDQLKNYVNDLVPEFLPAPEDVETNCEWSAFSCFQKA | | |
| | | QLKSANTGNNERIINESIKKLKRKPPSTNPGQRQKDRLTCP | | |
| | | SCDSYEKKPPKEFLERFKSLLQKMIHQHLSSRTHGSEDS | | |
| IL-21_4948 | T110D, R115D, | MRSSPGNMERIVICLMVIFLGTLVHKSSSQGQDRHMIRMRQ | 1.162 | 1690 |
| | R119T, T121E | LIDIVDQLKNYVNDLVPEFLPAPEDVETNCEWSAFSCFQKA | | |
| | | QLKSANTGNNERIINVSIKKLKRKPPSDNAGRDQKHTLECP | | |
| | | SCDSYEKKPPKEFLERFKSLLQKMIHQHLSSRTHGSEDS | | |
| IL-21_5380 | N70G, P107V, | MRSSPGNMERIVICLMVIFLGTLVHKSSSQGQDRHMIRMRQ | 1.162 | 1691 |
| | H118D, T121E | LIDIVDQLKNYVNDLVPEFLPAPEDVETGCEWSAFSCFQKA | | |
| | | QLKSANTGNNERIINVSIKKLKRKVPSTNAGRRQKDRLECP | | |
| | | SCDSYEKKPPKEFLERFKSLLQKMIHQHLSSRTHGSEDS | | |
| IL-21_4933 | A112P, R114G, | MRSSPGNMERIVICLMVIFLGTLVHKSSSQGQDRHMIRMRQ | 1.163 | 1692 |
| | R115D, R119T | LIDIVDQLKNYVNDLVPEFLPAPEDVETNCEWSAFSCFQKA | | |
| | | QLKSANTGNNERIINVSIKKLKRKPPSTNPGGDQKHTLTCP | | |
| | | SCDSYEKKPPKEFLERFKSLLQKMIHQHLSSRTHGSEDS | | |
| IL-21_5354 | N70G, T110D, | MRSSPGNMERIVICLMVIFLGTLVHKSSSQGQDRHMIRMRQ | 1.166 | 1693 |
| | H118D, T121E | LIDIVDQLKNYVNDLVPEFLPAPEDVETGCEWSAFSCFQKA | | |
| | | QLKSANTGNNERIINVSIKKLKRKPPSDNAGRRQKDRLECP | | |
| | | SCDSYEKKPPKEFLERFKSLLQKMIHQHLSSRTHGSEDS | | |
| IL-21_5738 | N70G, T110D, | MRSSPGNMERIVICLMVIFLGTLVHKSSSQGQDRHMIRMRQ | 1.167 | 1694 |
| | R115D | LIDIVDQLKNYVNDLVPEFLPAPEDVETGCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6056 | H35F, I45T, | MRSSPGNMERIVICLMVIFLGTLVHKSSSQGQDREMIRMRQ | 1.168 | 1695 |
| | R114Q, R115G | LIDTVDQLKNYVNDLVPEFLPAPEDVETNCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_6219 | H35I, I45T, | MRSSPGNMERIVICLMVIFLGTLVHKSSSQGQDRIMIRMRQ | 1.169 | 1696 |
| | V98E, R115G | LIDTVDQLKNYVNDLVPEFLPAPEDVETNCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_5148 | V98E, P107V, | MRSSPGNMERIVICLMVIFLGTLVHKSSSQGQDRHMIRMRQ | 1.17 | 1697 |
| | A112P, R119T | LIDIVDQLKNYVNDLVPEFLPAPEDVETNCEWSAFSCFQKA | | |
| | | QLKSANTGNNERIINESIKKLKRKVPSTNPGRRQKHTLTCP | | |
| | | SCDSYEKKPPKEFLERFKSLLQKMIHQHLSSRTHGSEDS | | |
| IL-21_5845 | N70G, K81D | MRSSPGNMERIVICLMVIFLGTLVHKSSSQGQDRHMIRMRQ | 1.17 | 1698 |
| | | LIDIVDQLKNYVNDLVPEFLPAPEDVETGCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5919 | I45T, N70G, | MRSSPGNMERIVICLMVIFLGTLVHKSSSQGQDRHMIRMRQ | 1.171 | 1699 |
| | R114Q, R115D | LIDTVDQLKNYVNDLVPEFLPAPEDVETGCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_4996 | P107V, R114G, | MRSSPGNMERIVICLMVIFLGTLVHKSSSQGQDRHMIRMRQ | 1.172 | 1700 |
| | H118D, T121E | LIDIVDQLKNYVNDLVPEFLPAPEDVETNCEWSAFSCFQKA | | |
| | | QLKSANTGNNERIINVSIKKLKRKVPSTNAGGRQKDRLECP | | |
| | | SCDSYEKKPPKEFLERFKSLLQKMIHQHLSSRTHGSEDS | | |
| IL-21_6272 | I45T, K81D, | MRSSPGNMERIVICLMVIFLGTLVHKSSSQGQDRHMIRMRQ | 1.174 | 1701 |
| | R114Q | LIDTVDQLKNYVNDLVPEFLPAPEDVETNCEWSAFSCFQDA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5432 | N70G, V98E, | MRSSPGNMERIVICLMVIFLGTLVHKSSSQGQDRHMIRMRQ | 1.175 | 1702 |
| | A112P, T121E | LIDIVDQLKNYVNDLVPEFLPAPEDVETGCEWSAFSCFQKA | | |
| | | QLKSANTGNNERIINESIKKLKRKPPSTNPGRRQKHRLECP | | |
| | | SCDSYEKKPPKEFLERFKSLLQKMIHQHLSSRTHGSEDS | | |
| IL-21_6111 | H35I, P107V, | MRSSPGNMERIVICLMVIFLGTLVHKSSSQGQDRIMIRMRQ | 1.175 | 1703 |
| | R114Q, R115G | LIDIVDQLKNYVNDLVPEFLPAPEDVETNCEWSAFSCFQKA | | |
| | | QLKSANTGNNERIINVSIKKLKRKVPSTNAGQGQKHRLTCP | | |
| | | SCDSYEKKPPKEFLERFKSLLQKMIHQHLSSRTHGSEDS | | |
| IL-21_4944 | T110D, H118D, | MRSSPGNMERIVICLMVIFLGTLVHKSSSQGQDRHMIRMRQ | 1.18 | 1704 |
| | R119T, T121E | LIDIVDQLKNYVNDLVPEFLPAPEDVETNCEWSAFSCFQKA | | |
| | | QLKSANTGNNERIINVSIKKLKRKPPSDNAGRRQKDTLECP | | |
| | | SCDSYEKKPPKEFLERFKSLLQKMIHQHLSSRTHGSEDS | | |
| IL-21_5925 | I45T, N70G, | MRSSPGNMERIVICLMVIFLGTLVHKSSSQGQDRHMIRMRQ | 1.183 | 1705 |
| | T110D, R115D | LIDTVDQLKNYVNDLVPEFLPAPEDVETGCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6230 | H35I, I45T, | MRSSPGNMERIVICLMVIFLGTLVHKSSSQGQDRIMIRMRQ | 1.183 | 1706 |
| | K81D, A112P | LIDTVDQLKNYVNDLVPEFLPAPEDVETNCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_4942 | A112P, R114Q, | MRSSPGNMERIVICLMVIFLGTLVHKSSSQGQDRHMIRMRQ | 1.184 | 1707 |
| | R115D, R119T | LIDIVDQLKNYVNDLVPEFLPAPEDVETNCEWSAFSCFQKA | | |
| | | QLKSANTGNNERIINVSIKKLKRKPPSTNPGQDQKHTLTCP | | |
| | | SCDSYEKKPPKEFLERFKSLLQKMIHQHLSSRTHGSEDS | | |
| IL-21_5917 | I45T, N70G, | MRSSPGNMERIVICLMVIFLGTLVHKSSSQGQDRHMIRMRQ | 1.184 | 1708 |
| | R114G, R115D | LIDTVDQLKNYVNDLVPEFLPAPEDVETGCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5655 | K81D, R119T, | MRSSPGNMERIVICLMVIFLGTLVHKSSSQGQDRHMIRMRQ | 1.185 | 1709 |
| | T121E | LIDIVDQLKNYVNDLVPEFLPAPEDVETNCEWSAFSCFQDA | | |
| | | QLKSANTGNNERIINVSIKKLKRKPPSTNAGRRQKHTLECP | | |
| | | SCDSYEKKPPKEFLERFKSLLQKMIHQHLSSRTHGSEDS | | |
| IL-21_5559 | T110D, R115D, | MRSSPGNMERIVICLMVIFLGTLVHKSSSQGQDRHMIRMRQ | 1.185 | 1710 |
| | T121E | LIDIVDQLKNYVNDLVPEFLPAPEDVETNCEWSAFSCFQKA | | |
| | | QLKSANTGNNERIINVSIKKLKRKPPSDNAGRDQKHRLECP | | |
| | | SCDSYEKKPPKEFLERFKSLLQKMIHQHLSSRTHGSEDS | | |
| IL-21_5357 | N70G, T110D, | MRSSPGNMERIVICLMVIFLGTLVHKSSSQGQDRHMIRMRQ | 1.185 | 1711 |
| | R115G, R119T | LIDIVDQLKNYVNDLVPEFLPAPEDVETGCEWSAFSCFQKA | | |
| | | QLKSANTGNNERIINVSIKKLKRKPPSDNAGRGQKHTLTCP | | |
| | | SCDSYEKKPPKEFLERFKSLLQKMIHQHLSSRTHGSEDS | | |
| IL-21_5390 | N70G, P107V, | MRSSPGNMERIVICLMVIFLGTLVHKSSSQGQDRHMIRMRQ | 1.186 | 1712 |
| | R114G, H118D | LIDIVDQLKNYVNDLVPEFLPAPEDVETGCEWSAFSCFQKA | | |
| | | QLKSANTGNNERIINVSIKKLKRKVPSTNAGGRQKDRLTCP | | |
| | | SCDSYEKKPPKEFLERFKSLLQKMIHQHLSSRTHGSEDS | | |
| IL-21_5364 | N70G, T110D, | MRSSPGNMERIVICLMVIFLGTLVHKSSSQGQDRHMIRMRQ | 1.187 | 1713 |
| | R114G, H118D | LIDIVDQLKNYVNDLVPEFLPAPEDVETGCEWSAFSCFQKA | | |
| | | QLKSANTGNNERIINVSIKKLKRKPPSDNAGGRQKDRLTCP | | |
| | | SCDSYEKKPPKEFLERFKSLLQKMIHQHLSSRTHGSEDS | | |
| IL-21_6112 | H35I, P107V, | MRSSPGNMERIVICLMVIFLGTLVHKSSSQGQDRIMIRMRQ | 1.187 | 1714 |
| | R114Q, R115D | LIDIVDQLKNYVNDLVPEFLPAPEDVETNCEWSAFSCFQKA | | |
| | | QLKSANTGNNERIINVSIKKLKRKVPSTNAGQDQKHRLTCP | | |
| | | SCDSYEKKPPKEFLERFKSLLQKMIHQHLSSRTHGSEDS | | |
| IL-21_5833 | K81D, P107V | MRSSPGNMERIVICLMVIFLGTLVHKSSSQGQDRHMIRMRQ | 1.188 | 1715 |
| | | LIDIVDQLKNYVNDLVPEFLPAPEDVETNCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5946 | I45T, N70G, | MRSSPGNMERIVICLMVIFLGTLVHKSSSQGQDRHMIRMRQ | 1.19 | 1716 |
| | K81D, A112P | LIDTVDQLKNYVNDLVPEFLPAPEDVETGCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5055 | P107V, T110D, | MRSSPGNMERIVICLMVIFLGTLVHKSSSQGQDRHMIRMRQ | 1.19 | 1717 |
| | A112P, R115D | LIDIVDQLKNYVNDLVPEFLPAPEDVETNCEWSAFSCFQKA | | |
| | | QLKSANTGNNERIINVSIKKLKRKVPSDNPGRDQKHRLTCP | | |
| | | SCDSYEKKPPKEFLERFKSLLQKMIHQHLSSRTHGSEDS | | |
| IL-21_6379 | H35I, N70G, | MRSSPGNMERIVICLMVIFLGTLVHKSSSQGQDRIMIRMRQ | 1.19 | 1718 |
| | V98E | LIDIVDQLKNYVNDLVPEFLPAPEDVETGCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5327 | N70G, R114Q, | MRSSPGNMERIVICLMVIFLGTLVHKSSSQGQDRHMIRMRQ | 1.192 | 1719 |
| | H118D, R119T | LIDIVDQLKNYVNDLVPEFLPAPEDVETGCEWSAFSCFQKA | | |
| | | QLKSANTGNNERIINVSIKKLKRKPPSTNAGQRQKDTLTCP | | |
| | | SCDSYEKKPPKEFLERFKSLLQKMIHQHLSSRTHGSEDS | | |
| IL-21_5005 | P107V, R114Q, | MRSSPGNMERIVICLMVIFLGTLVHKSSSQGQDRHMIRMRQ | 1.193 | 1720 |
| | H118D, T121E | LIDIVDQLKNYVNDLVPEFLPAPEDVETNCEWSAFSCFQKA | | |
| | | QLKSANTGNNERIINVSIKKLKRKVPSTNAGQRQKDRLECP | | |
| | | SCDSYEKKPPKEFLERFKSLLQKMIHQHLSSRTHGSEDS | | |
| IL-21_5476 | N70G, K81D, | MRSSPGNMERIVICLMVIFLGTLVHKSSSQGQDRHMIRMRQ | 1.194 | 1721 |
| | A112P, R119T | LIDIVDQLKNYVNDLVPEFLPAPEDVETGCEWSAFSCFQDA | | |
| | | QLKSANTGNNERIINVSIKKLKRKPPSTNPGRRQKHTLTCP | | |
| | | SCDSYEKKPPKEFLERFKSLLQKMIHQHLSSRTHGSEDS | | |
| IL-21_6031 | H35F, N70G, | MRSSPGNMERIVICLMVIFLGTLVHKSSSQGQDREMIRMRQ | 1.194 | 1722 |
| | T110D, R114Q | LIDIVDQLKNYVNDLVPEFLPAPEDVETGCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_6232 | H35I, I45T, | MRSSPGNMERIVICLMVIFLGTLVHKSSSQGQDRIMIRMRQ | 1.195 | 1723 |
| | K81D, P107V | LIDTVDQLKNYVNDLVPEFLPAPEDVETNCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5034 | P107V, T110D, | MRSSPGNMERIVICLMVIFLGTLVHKSSSQGQDRHMIRMRQ | 1.195 | 1724 |
| | H118D, R119T | LIDIVDQLKNYVNDLVPEFLPAPEDVETNCEWSAFSCFQKA | | |
| | | QLKSANTGNNERIINVSIKKLKRKVPSDNAGRRQKDTLTCP | | |
| | | SCDSYEKKPPKEFLERFKSLLQKMIHQHLSSRTHGSEDS | | |
| IL-21_6370 | H35I, K81D, | MRSSPGNMERIVICLMVIFLGTLVHKSSSQGQDRIMIRMRQ | 1.196 | 1725 |
| | P107V | LIDIVDQLKNYVNDLVPEFLPAPEDVETNCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5008 | P107V, R114Q, | MRSSPGNMERIVICLMVIFLGTLVHKSSSQGQDRHMIRMRQ | 1.196 | 1726 |
| | R115G, R119T | LIDIVDQLKNYVNDLVPEFLPAPEDVETNCEWSAFSCFQKA | | |
| | | QLKSANTGNNERIINVSIKKLKRKVPSTNAGQGQKHTLTCP | | |
| | | SCDSYEKKPPKEFLERFKSLLQKMIHQHLSSRTHGSEDS | | |
| IL-21_4916 | R114Q, R115D, | MRSSPGNMERIVICLMVIFLGTLVHKSSSQGQDRHMIRMRQ | 1.199 | 1727 |
| | R119T, T121E | LIDIVDQLKNYVNDLVPEFLPAPEDVETNCEWSAFSCFQKA | | |
| | | QLKSANTGNNERIINVSIKKLKRKPPSTNAGQDQKHTLECP | | |
| | | SCDSYEKKPPKEFLERFKSLLQKMIHQHLSSRTHGSEDS | | |
| IL-21_5007 | P107V, R114Q, | MRSSPGNMERIVICLMVIFLGTLVHKSSSQGQDRHMIRMRQ | 1.201 | 1728 |
| | R115G, T121E | LIDIVDQLKNYVNDLVPEFLPAPEDVETNCEWSAFSCFQKA | | |
| | | QLKSANTGNNERIINVSIKKLKRKVPSTNAGQGQKHRLECP | | |
| | | SCDSYEKKPPKEFLERFKSLLQKMIHQHLSSRTHGSEDS | | |
| IL-21_6189 | H35I, N70G, | MRSSPGNMERIVICLMVIFLGTLVHKSSSQGQDRIMIRMRQ | 1.201 | 1729 |
| | V98E, A112P | LIDIVDQLKNYVNDLVPEFLPAPEDVETGCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5352 | N70G, A112P, | MRSSPGNMERIVICLMVIFLGTLVHKSSSQGQDRHMIRMRQ | 1.201 | 1730 |
| | R114Q, R115D | LIDIVDQLKNYVNDLVPEFLPAPEDVETGCEWSAFSCFQKA | | |
| | | QLKSANTGNNERIINVSIKKLKRKPPSTNPGQDQKHRLTCP | | |
| | | SCDSYEKKPPKEFLERFKSLLQKMIHQHLSSRTHGSEDS | | |
| IL-21_6030 | H35F, N70G, | MRSSPGNMERIVICLMVIFLGTLVHKSSSQGQDRFMIRMRQ | 1.202 | 1731 |
| | T110D, R114G | LIDIVDQLKNYVNDLVPEFLPAPEDVETGCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5612 | V98E, R119T, | MRSSPGNMERIVICLMVIFLGTLVHKSSSQGQDRHMIRMRQ | 1.203 | 1732 |
| | T121E | LIDIVDQLKNYVNDLVPEFLPAPEDVETNCEWSAFSCFQKA | | |
| | | QLKSANTGNNERIINESIKKLKRKPPSTNAGRRQKHTLECP | | |
| | | SCDSYEKKPPKEFLERFKSLLQKMIHQHLSSRTHGSEDS | | |
| IL-21_5964 | H35F, P107V, | MRSSPGNMERIVICLMVIFLGTLVHKSSSQGQDREMIRMRQ | 1.204 | 1733 |
| | R114Q, R115G | LIDIVDQLKNYVNDLVPEFLPAPEDVETNCEWSAFSCFQKA | | |
| | | QLKSANTGNNERIINVSIKKLKRKVPSTNAGQGQKHRLTCP | | |
| | | SCDSYEKKPPKEFLERFKSLLQKMIHQHLSSRTHGSEDS | | |
| IL-21_6103 | H35I, T110D, | MRSSPGNMERIVICLMVIFLGTLVHKSSSQGQDRIMIRMRQ | 1.204 | 1734 |
| | R114Q, R115G | LIDIVDQLKNYVNDLVPEFLPAPEDVETNCEWSAFSCFQKA | | |
| | | QLKSANTGNNERIINVSIKKLKRKPPSDNAGQGQKHRLTCP | | |
| | | SCDSYEKKPPKEFLERFKSLLQKMIHQHLSSRTHGSEDS | | |
| IL-21_5320 | N70G, R114G, | MRSSPGNMERIVICLMVIFLGTLVHKSSSQGQDRHMIRMRQ | 1.205 | 1735 |
| | R115G, R119T | LIDIVDQLKNYVNDLVPEFLPAPEDVETGCEWSAFSCFQKA | | |
| | | QLKSANTGNNERIINVSIKKLKRKPPSTNAGGGQKHTLTCP | | |
| | | SCDSYEKKPPKEFLERFKSLLQKMIHQHLSSRTHGSEDS | | |
| IL-21_5514 | R115D, H118D, | MRSSPGNMERIVICLMVIFLGTLVHKSSSQGQDRHMIRMRQ | 1.205 | 1736 |
| | T121E | LIDIVDQLKNYVNDLVPEFLPAPEDVETNCEWSAFSCFQKA | | |
| | | QLKSANTGNNERIINVSIKKLKRKPPSTNAGRDQKDRLECP | | |
| | | SCDSYEKKPPKEFLERFKSLLQKMIHQHLSSRTHGSEDS | | |
| IL-21_5351 | N70G, A112P, | MRSSPGNMERIVICLMVIFLGTLVHKSSSQGQDRHMIRMRQ | 1.205 | 1737 |
| | R114Q, R115G | LIDIVDQLKNYVNDLVPEFLPAPEDVETGCEWSAFSCFQKA | | |
| | | QLKSANTGNNERIINVSIKKLKRKPPSTNPGQGQKHRLTCP | | |
| | | SCDSYEKKPPKEFLERFKSLLQKMIHQHLSSRTHGSEDS | | |
| IL-21_4958 | T110D, R114G, | MRSSPGNMERIVICLMVIFLGTLVHKSSSQGQDRHMIRMRQ | 1.206 | 1738 |
| | R115D, R119T | LIDIVDQLKNYVNDLVPEFLPAPEDVETNCEWSAFSCFQKA | | |
| | | QLKSANTGNNERIINVSIKKLKRKPPSDNAGGDQKHTLTCP | | |
| | | SCDSYEKKPPKEFLERFKSLLQKMIHQHLSSRTHGSEDS | | |
| IL-21_5381 | N70G, P107V, | MRSSPGNMERIVICLMVIFLGTLVHKSSSQGQDRHMIRMRQ | 1.207 | 1739 |
| | H118D, R119T | LIDIVDQLKNYVNDLVPEFLPAPEDVETGCEWSAFSCFQKA | | |
| | | QLKSANTGNNERIINVSIKKLKRKVPSTNAGRRQKDTLTCP | | |
| | | SCDSYEKKPPKEFLERFKSLLQKMIHQHLSSRTHGSEDS | | |
| IL-21_5532 | R114Q, R115D, | MRSSPGNMERIVICLMVIFLGTLVHKSSSQGQDRHMIRMRQ | 1.207 | 1740 |
| | R119T | LIDIVDQLKNYVNDLVPEFLPAPEDVETNCEWSAFSCFQKA | | |
| | | QLKSANTGNNERIINVSIKKLKRKPPSTNAGQDQKHTLTCP | | |
| | | SCDSYEKKPPKEFLERFKSLLQKMIHQHLSSRTHGSEDS | | |
| IL-21_5967 | H35F, P107V, | MRSSPGNMERIVICLMVIFLGTLVHKSSSQGQDRFMIRMRQ | 1.207 | 1741 |
| | A112P, R115D | LIDIVDQLKNYVNDLVPEFLPAPEDVETNCEWSAFSCFQKA | | |
| | | QLKSANTGNNERIINVSIKKLKRKVPSTNPGRDQKHRLTCP | | |
| | | SCDSYEKKPPKEFLERFKSLLQKMIHQHLSSRTHGSEDS | | |
| IL-21_5375 | N70G, T110D, | MRSSPGNMERIVICLMVIFLGTLVHKSSSQGQDRHMIRMRQ | 1.207 | 1742 |
| | A112P, R115G | LIDIVDQLKNYVNDLVPEFLPAPEDVETGCEWSAFSCFQKA | | |
| | | QLKSANTGNNERIINVSIKKLKRKPPSDNPGRGQKHRLTCP | | |
| | | SCDSYEKKPPKEFLERFKSLLQKMIHQHLSSRTHGSEDS | | |
| IL-21_4908 | R114G, R115G, | MRSSPGNMERIVICLMVIFLGTLVHKSSSQGQDRHMIRMRQ | 1.208 | 1743 |
| | H118D, R119T | LIDIVDQLKNYVNDLVPEFLPAPEDVETNCEWSAFSCFQKA | | |
| | | QLKSANTGNNERIINVSIKKLKRKPPSTNAGGGQKDTLTCP | | |
| | | SCDSYEKKPPKEFLERFKSLLQKMIHQHLSSRTHGSEDS | | |
| IL-21_5826 | K81D, H118D | MRSSPGNMERIVICLMVIFLGTLVHKSSSQGQDRHMIRMRQ | 1.209 | 1744 |
| | | LIDIVDQLKNYVNDLVPEFLPAPEDVETNCEWSAFSCFQDA | | |
| | | QLKSANTGNNERIINVSIKKLKRKPPSTNAGRRQKDRLTCP | | |
| | | SCDSYEKKPPKEFLERFKSLLQKMIHQHLSSRTHGSEDS | | |
| IL-21_5311 | N70G, R115G, | MRSSPGNMERIVICLMVIFLGTLVHKSSSQGQDRHMIRMRQ | 1.209 | 1745 |
| | H118D, T121E | LIDIVDQLKNYVNDLVPEFLPAPEDVETGCEWSAFSCFQKA | | |
| | | QLKSANTGNNERIINVSIKKLKRKPPSTNAGRGQKDRLECP | | |
| | | SCDSYEKKPPKEFLERFKSLLQKMIHQHLSSRTHGSEDS | | |
| IL-21_4912 | R114Q, H118D, | MRSSPGNMERIVICLMVIFLGTLVHKSSSQGQDRHMIRMRQ | 1.212 | 1746 |
| | R119T, T121E | LIDIVDQLKNYVNDLVPEFLPAPEDVETNCEWSAFSCFQKA | | |
| | | QLKSANTGNNERIINVSIKKLKRKPPSTNAGQRQKDTLECP | | |
| | | SCDSYEKKPPKEFLERFKSLLQKMIHQHLSSRTHGSEDS | | |
| IL-21_4999 | P107V, R114G, | MRSSPGNMERIVICLMVIFLGTLVHKSSSQGQDRHMIRMRQ | 1.213 | 1747 |
| | R115G, R119T | LIDIVDQLKNYVNDLVPEFLPAPEDVETNCEWSAFSCFQKA | | |
| | | QLKSANTGNNERIINVSIKKLKRKVPSTNAGGGQKHTLTCP | | |
| | | SCDSYEKKPPKEFLERFKSLLQKMIHQHLSSRTHGSEDS | | |
| IL-21_5689 | K81D, P107V, | MRSSPGNMERIVICLMVIFLGTLVHKSSSQGQDRHMIRMRQ | 1.213 | 1748 |
| | T121E | LIDIVDQLKNYVNDLVPEFLPAPEDVETNCEWSAFSCFQDA | | |
| | | QLKSANTGNNERIINVSIKKLKRKVPSTNAGRRQKHRLECP | | |
| | | SCDSYEKKPPKEFLERFKSLLQKMIHQHLSSRTHGSEDS | | |
| IL-21_6269 | I45T, K81D, | MRSSPGNMERIVICLMVIFLGTLVHKSSSQGQDRHMIRMRQ | 1.213 | 1749 |
| | R115G | LIDTVDQLKNYVNDLVPEFLPAPEDVETNCEWSAFSCFQDA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5410 | N70G, P107V, | MRSSPGNMERIVICLMVIFLGTLVHKSSSQGQDRHMIRMRQ | 1.213 | 1750 |
| | T110D, R114G | LIDIVDQLKNYVNDLVPEFLPAPEDVETGCEWSAFSCFQKA | | |
| | | QLKSANTGNNERIINVSIKKLKRKVPSDNAGGRQKHRLTCP | | |
| | | SCDSYEKKPPKEFLERFKSLLQKMIHQHLSSRTHGSEDS | | |
| IL-21_6078 | H35F, I45T, | MRSSPGNMERIVICLMVIFLGTLVHKSSSQGQDRFMIRMRQ | 1.214 | 1751 |
| | V98E, T110D | LIDTVDQLKNYVNDLVPEFLPAPEDVETNCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5859 | I45T, P107V, | MRSSPGNMERIVICLMVIFLGTLVHKSSSQGQDRHMIRMRQ | 1.218 | 1752 |
| | R114G, R115D | LIDTVDQLKNYVNDLVPEFLPAPEDVETNCEWSAFSCFQKA | | |
| | | QLKSANTGNNERIINVSIKKLKRKVPSTNAGGDQKHRLTCP | | |
| | | SCDSYEKKPPKEFLERFKSLLQKMIHQHLSSRTHGSEDS | | |
| IL-21_4961 | T110D, R114Q, | MRSSPGNMERIVICLMVIFLGTLVHKSSSQGQDRHMIRMRQ | 1.222 | 1753 |
| | H118D, T121E | LIDIVDQLKNYVNDLVPEFLPAPEDVETNCEWSAFSCFQKA | | |
| | | QLKSANTGNNERIINVSIKKLKRKPPSDNAGQRQKDRLECP | | |
| | | SCDSYEKKPPKEFLERFKSLLQKMIHQHLSSRTHGSEDS | | |
| IL-21_6341 | H35I, R114Q, | MRSSPGNMERIVICLMVIFLGTLVHKSSSQGQDRIMIRMRQ | 1.222 | 1754 |
| | R115D | LIDIVDQLKNYVNDLVPEFLPAPEDVETNCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_6176 | H35I, N70G, | MRSSPGNMERIVICLMVIFLGTLVHKSSSQGQDRIMIRMRQ | 1.222 | 1755 |
| | T110D, R115D | LIDIVDQLKNYVNDLVPEFLPAPEDVETGCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6180 | H35I, N70G, | MRSSPGNMERIVICLMVIFLGTLVHKSSSQGQDRIMIRMRQ | 1.223 | 1756 |
| | P107V, R115D | LIDIVDQLKNYVNDLVPEFLPAPEDVETGCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5719 | N70G, R114G, | MRSSPGNMERIVICLMVIFLGTLVHKSSSQGQDRHMIRMRQ | 1.224 | 1757 |
| | H118D | LIDIVDQLKNYVNDLVPEFLPAPEDVETGCEWSAFSCFQKA | | |
| | | QLKSANTGNNERIINVSIKKLKRKPPSTNAGGRQKDRLTCP | | |
| | | SCDSYEKKPPKEFLERFKSLLQKMIHQHLSSRTHGSEDS | | |
| IL-21_5020 | P107V, A112P, | MRSSPGNMERIVICLMVIFLGTLVHKSSSQGQDRHMIRMRQ | 1.225 | 1758 |
| | R115D, R119T | LIDIVDQLKNYVNDLVPEFLPAPEDVETNCEWSAFSCFQKA | | |
| | | QLKSANTGNNERIINVSIKKLKRKVPSTNPGRDQKHTLTCP | | |
| | | SCDSYEKKPPKEFLERFKSLLQKMIHQHLSSRTHGSEDS | | |
| IL-21_6368 | H35I, K81D, | MRSSPGNMERIVICLMVIFLGTLVHKSSSQGQDRIMIRMRQ | 1.226 | 1759 |
| | A112P | LIDIVDQLKNYVNDLVPEFLPAPEDVETNCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5632 | V98E, A112P, | MRSSPGNMERIVICLMVIFLGTLVHKSSSQGQDRHMIRMRQ | 1.226 | 1760 |
| | R119T | LIDIVDQLKNYVNDLVPEFLPAPEDVETNCEWSAFSCFQKA | | |
| | | QLKSANTGNNERIINESIKKLKRKPPSTNPGRRQKHTLTCP | | |
| | | SCDSYEKKPPKEFLERFKSLLQKMIHQHLSSRTHGSEDS | | |
| IL-21_5384 | N70G, P107V, | MRSSPGNMERIVICLMVIFLGTLVHKSSSQGQDRHMIRMRQ | 1.226 | 1761 |
| | R115G, H118D | LIDIVDQLKNYVNDLVPEFLPAPEDVETGCEWSAFSCFQKA | | |
| | | QLKSANTGNNERIINVSIKKLKRKVPSTNAGRGQKDRLTCP | | |
| | | SCDSYEKKPPKEFLERFKSLLQKMIHQHLSSRTHGSEDS | | |
| IL-21_6178 | H35I, N70G, | MRSSPGNMERIVICLMVIFLGTLVHKSSSQGQDRIMIRMRQ | 1.228 | 1762 |
| | T110D, R114Q | LIDIVDQLKNYVNDLVPEFLPAPEDVETGCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_6262 | I45T, V98E, | MRSSPGNMERIVICLMVIFLGTLVHKSSSQGQDRHMIRMRQ | 1.228 | 1763 |
| | R115G | LIDTVDQLKNYVNDLVPEFLPAPEDVETNCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_4931 | A112P, R114G, | MRSSPGNMERIVICLMVIFLGTLVHKSSSQGQDRHMIRMRQ | 1.229 | 1764 |
| | R115G, H118D | LIDIVDQLKNYVNDLVPEFLPAPEDVETNCEWSAFSCFQKA | | |
| | | QLKSANTGNNERIINVSIKKLKRKPPSTNPGGGQKDRLTCP | | |
| | | SCDSYEKKPPKEFLERFKSLLQKMIHQHLSSRTHGSEDS | | |
| IL-21_6347 | H35I, T110D, | MRSSPGNMERIVICLMVIFLGTLVHKSSSQGQDRIMIRMRQ | 1.23 | 1765 |
| | R115D | LIDIVDQLKNYVNDLVPEFLPAPEDVETNCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_5512 | R115G, H118D, | MRSSPGNMERIVICLMVIFLGTLVHKSSSQGQDRHMIRMRQ | 1.233 | 1766 |
| | R119T | LIDIVDQLKNYVNDLVPEFLPAPEDVETNCEWSAFSCFQKA | | |
| | | QLKSANTGNNERIINVSIKKLKRKPPSTNAGRGQKDTLTCP | | |
| | | SCDSYEKKPPKEFLERFKSLLQKMIHQHLSSRTHGSEDS | | |
| IL-21_6068 | H35F, I45T, | MRSSPGNMERIVICLMVIFLGTLVHKSSSQGQDREMIRMRQ | 1.235 | 1767 |
| | P107V, R115D | LIDTVDQLKNYVNDLVPEFLPAPEDVETNCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5680 | K81D, A112P, | MRSSPGNMERIVICLMVIFLGTLVHKSSSQGQDRHMIRMRQ | 1.235 | 1768 |
| | R114Q | LIDIVDQLKNYVNDLVPEFLPAPEDVETNCEWSAFSCFQDA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_6117 | H35I, P107V, | MRSSPGNMERIVICLMVIFLGTLVHKSSSQGQDRIMIRMRQ | 1.236 | 1769 |
| | T110D, R115G | LIDIVDQLKNYVNDLVPEFLPAPEDVETNCEWSAFSCFQKA | | |
| | | QLKSANTGNNERIINVSIKKLKRKVPSDNAGRGQKHRLTCP | | |
| | | SCDSYEKKPPKEFLERFKSLLQKMIHQHLSSRTHGSEDS | | |
| IL-21_6114 | H35I, P107V, | MRSSPGNMERIVICLMVIFLGTLVHKSSSQGQDRIMIRMRQ | 1.238 | 1770 |
| | A112P, R115D | LIDIVDQLKNYVNDLVPEFLPAPEDVETNCEWSAFSCFQKA | | |
| | | QLKSANTGNNERIINVSIKKLKRKVPSTNPGRDQKHRLTCP | | |
| | | SCDSYEKKPPKEFLERFKSLLQKMIHQHLSSRTHGSEDS | | |
| IL-21_6373 | H35I, N70G, | MRSSPGNMERIVICLMVIFLGTLVHKSSSQGQDRIMIRMRQ | 1.239 | 1771 |
| | R115D | LIDIVDQLKNYVNDLVPEFLPAPEDVETGCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5004 | P107V, R114Q, | MRSSPGNMERIVICLMVIFLGTLVHKSSSQGQDRHMIRMRQ | 1.239 | 1772 |
| | R119T, T121E | LIDIVDQLKNYVNDLVPEFLPAPEDVETNCEWSAFSCFQKA | | |
| | | QLKSANTGNNERIINVSIKKLKRKVPSTNAGQRQKHTLECP | | |
| | | SCDSYEKKPPKEFLERFKSLLQKMIHQHLSSRTHGSEDS | | |
| IL-21_5586 | P107V, R115D, | MRSSPGNMERIVICLMVIFLGTLVHKSSSQGQDRHMIRMRQ | 1.239 | 1773 |
| | R119T | LIDIVDQLKNYVNDLVPEFLPAPEDVETNCEWSAFSCFQKA | | |
| | | QLKSANTGNNERIINVSIKKLKRKVPSTNAGRDQKHTLTCP | | |
| | | SCDSYEKKPPKEFLERFKSLLQKMIHQHLSSRTHGSEDS | | |
| IL-21_5368 | N70G, T110D, | MRSSPGNMERIVICLMVIFLGTLVHKSSSQGQDRHMIRMRQ | 1.239 | 1774 |
| | R114Q, R119T | LIDIVDQLKNYVNDLVPEFLPAPEDVETGCEWSAFSCFQKA | | |
| | | QLKSANTGNNERIINVSIKKLKRKPPSDNAGQRQKHTLTCP | | |
| | | SCDSYEKKPPKEFLERFKSLLQKMIHQHLSSRTHGSEDS | | |
| IL-21_5036 | P107V, T110D, | MRSSPGNMERIVICLMVIFLGTLVHKSSSQGQDRHMIRMRQ | 1.24 | 1775 |
| | R115G, R119T | LIDIVDQLKNYVNDLVPEFLPAPEDVETNCEWSAFSCFQKA | | |
| | | QLKSANTGNNERIINVSIKKLKRKVPSDNAGRGQKHTLTCP | | |
| | | SCDSYEKKPPKEFLERFKSLLQKMIHQHLSSRTHGSEDS | | |
| IL-21_5992 | H35F, V98E, | MRSSPGNMERIVICLMVIFLGTLVHKSSSQGQDREMIRMRQ | 1.241 | 1776 |
| | P107V, A112P | LIDIVDQLKNYVNDLVPEFLPAPEDVETNCEWSAFSCFQKA | | |
| | | QLKSANTGNNERIINESIKKLKRKVPSTNPGRRQKHRLTCP | | |
| | | SCDSYEKKPPKEFLERFKSLLQKMIHQHLSSRTHGSEDS | | |
| IL-21_6100 | H35I, A112P, | MRSSPGNMERIVICLMVIFLGTLVHKSSSQGQDRIMIRMRQ | 1.242 | 1777 |
| | R114Q, R115D | LIDIVDQLKNYVNDLVPEFLPAPEDVETNCEWSAFSCFQKA | | |
| | | QLKSANTGNNERIINVSIKKLKRKPPSTNPGQDQKHRLTCP | | |
| | | SCDSYEKKPPKEFLERFKSLLQKMIHQHLSSRTHGSEDS | | |
| IL-21_4915 | R114Q, R115G, | MRSSPGNMERIVICLMVIFLGTLVHKSSSQGQDRHMIRMRQ | 1.242 | 1778 |
| | H118D, R119T | LIDIVDQLKNYVNDLVPEFLPAPEDVETNCEWSAFSCFQKA | | |
| | | QLKSANTGNNERIINVSIKKLKRKPPSTNAGQGQKDTLTCP | | |
| | | SCDSYEKKPPKEFLERFKSLLQKMIHQHLSSRTHGSEDS | | |
| IL-21_5326 | N70G, R114Q, | MRSSPGNMERIVICLMVIFLGTLVHKSSSQGQDRHMIRMRQ | 1.244 | 1779 |
| | H118D, T121E | LIDIVDQLKNYVNDLVPEFLPAPEDVETGCEWSAFSCFQKA | | |
| | | QLKSANTGNNERIINVSIKKLKRKPPSTNAGQRQKDRLECP | | |
| | | SCDSYEKKPPKEFLERFKSLLQKMIHQHLSSRTHGSEDS | | |
| IL-21_5033 | P107V, T110D, | MRSSPGNMERIVICLMVIFLGTLVHKSSSQGQDRHMIRMRQ | 1.244 | 1780 |
| | H118D, T121E | LIDIVDQLKNYVNDLVPEFLPAPEDVETNCEWSAFSCFQKA | | |
| | | QLKSANTGNNERIINVSIKKLKRKVPSDNAGRRQKDRLECP | | |
| | | SCDSYEKKPPKEFLERFKSLLQKMIHQHLSSRTHGSEDS | | |
| IL-21_6120 | H35I, P107V, | MRSSPGNMERIVICLMVIFLGTLVHKSSSQGQDRIMIRMRQ | 1.245 | 1781 |
| | T110D, R114Q | LIDIVDQLKNYVNDLVPEFLPAPEDVETNCEWSAFSCFQKA | | |
| | | QLKSANTGNNERIINVSIKKLKRKVPSDNAGQRQKHRLTCP | | |
| | | SCDSYEKKPPKEFLERFKSLLQKMIHQHLSSRTHGSEDS | | |
| IL-21_5411 | N70G, P107V, | MRSSPGNMERIVICLMVIFLGTLVHKSSSQGQDRHMIRMRQ | 1.245 | 1782 |
| | T110D, R114Q | LIDIVDQLKNYVNDLVPEFLPAPEDVETGCEWSAFSCFQKA | | |
| | | QLKSANTGNNERIINVSIKKLKRKVPSDNAGQRQKHRLTCP | | |
| | | SCDSYEKKPPKEFLERFKSLLQKMIHQHLSSRTHGSEDS | | |
| IL-21_5365 | N70G, T110D, | MRSSPGNMERIVICLMVIFLGTLVHKSSSQGQDRHMIRMRQ | 1.247 | 1783 |
| | R114G, R115G | LIDIVDQLKNYVNDLVPEFLPAPEDVETGCEWSAFSCFQKA | | |
| | | QLKSANTGNNERIINVSIKKLKRKPPSDNAGGGQKHRLTCP | | |
| | | SCDSYEKKPPKEFLERFKSLLQKMIHQHLSSRTHGSEDS | | |
| IL-21_5363 | N70G, T110D, | MRSSPGNMERIVICLMVIFLGTLVHKSSSQGQDRHMIRMRQ | 1.247 | 1784 |
| | R114G, R119T | LIDIVDQLKNYVNDLVPEFLPAPEDVETGCEWSAFSCFQKA | | |
| | | QLKSANTGNNERIINVSIKKLKRKPPSDNAGGRQKHTLTCP | | |
| | | SCDSYEKKPPKEFLERFKSLLQKMIHQHLSSRTHGSEDS | | |
| IL-21_5515 | R115D, H118D, | MRSSPGNMERIVICLMVIFLGTLVHKSSSQGQDRHMIRMRQ | 1.251 | 1785 |
| | R119T | LIDIVDQLKNYVNDLVPEFLPAPEDVETNCEWSAFSCFQKA | | |
| | | QLKSANTGNNERIINVSIKKLKRKPPSTNAGRDQKDTLTCP | | |
| | | SCDSYEKKPPKEFLERFKSLLQKMIHQHLSSRTHGSEDS | | |
| IL-21_5825 | K81D, R119T | MRSSPGNMERIVICLMVIFLGTLVHKSSSQGQDRHMIRMRQ | 1.251 | 1786 |
| | | LIDIVDQLKNYVNDLVPEFLPAPEDVETNCEWSAFSCFQDA | | |
| | | QLKSANTGNNERIINVSIKKLKRKPPSTNAGRRQKHTLTCP | | |
| | | SCDSYEKKPPKEFLERFKSLLQKMIHQHLSSRTHGSEDS | | |
| IL-21_4989 | P107V, R115G, | MRSSPGNMERIVICLMVIFLGTLVHKSSSQGQDRHMIRMRQ | 1.252 | 1787 |
| | R119T, T121E | LIDIVDQLKNYVNDLVPEFLPAPEDVETNCEWSAFSCFQKA | | |
| | | QLKSANTGNNERIINVSIKKLKRKVPSTNAGRGQKHTLECP | | |
| | | SCDSYEKKPPKEFLERFKSLLQKMIHQHLSSRTHGSEDS | | |
| IL-21_6308 | H35F, V98E, | MRSSPGNMERIVICLMVIFLGTLVHKSSSQGQDREMIRMRQ | 1.253 | 1788 |
| | A112P | LIDIVDQLKNYVNDLVPEFLPAPEDVETNCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5675 | K81D, A112P, | MRSSPGNMERIVICLMVIFLGTLVHKSSSQGQDRHMIRMRQ | 1.253 | 1789 |
| | R119T | LIDIVDQLKNYVNDLVPEFLPAPEDVETNCEWSAFSCFQDA | | |
| | | QLKSANTGNNERIINVSIKKLKRKPPSTNPGRRQKHTLTCP | | |
| | | SCDSYEKKPPKEFLERFKSLLQKMIHQHLSSRTHGSEDS | | |
| IL-21_5962 | H35F, P107V, | MRSSPGNMERIVICLMVIFLGTLVHKSSSQGQDRFMIRMRQ | 1.254 | 1790 |
| | R114G, R115G | LIDIVDQLKNYVNDLVPEFLPAPEDVETNCEWSAFSCFQKA | | |
| | | QLKSANTGNNERIINVSIKKLKRKVPSTNAGGGQKHRLTCP | | |
| | | SCDSYEKKPPKEFLERFKSLLQKMIHQHLSSRTHGSEDS | | |
| IL-21_5049 | P107V, T110D, | MRSSPGNMERIVICLMVIFLGTLVHKSSSQGQDRHMIRMRQ | 1.256 | 1791 |
| | R114Q, R115G | LIDIVDQLKNYVNDLVPEFLPAPEDVETNCEWSAFSCFQKA | | |
| | | QLKSANTGNNERIINVSIKKLKRKVPSDNAGQGQKHRLTCP | | |
| | | SCDSYEKKPPKEFLERFKSLLQKMIHQHLSSRTHGSEDS | | |
| IL-21_5454 | N70G, V98E, | MRSSPGNMERIVICLMVIFLGTLVHKSSSQGQDRHMIRMRQ | 1.256 | 1792 |
| | P107V, A112P | LIDIVDQLKNYVNDLVPEFLPAPEDVETGCEWSAFSCFQKA | | |
| | | QLKSANTGNNERIINESIKKLKRKVPSTNPGRRQKHRLTCP | | |
| | | SCDSYEKKPPKEFLERFKSLLQKMIHQHLSSRTHGSEDS | | |
| IL-21_4903 | R115G, H118D, | MRSSPGNMERIVICLMVIFLGTLVHKSSSQGQDRHMIRMRQ | 1.256 | 1793 |
| | R119T, T121E | LIDIVDQLKNYVNDLVPEFLPAPEDVETNCEWSAFSCFQKA | | |
| | | QLKSANTGNNERIINVSIKKLKRKPPSTNAGRGQKDTLECP | | |
| | | SCDSYEKKPPKEFLERFKSLLQKMIHQHLSSRTHGSEDS | | |
| IL-21_5696 | K81D, P107V, | MRSSPGNMERIVICLMVIFLGTLVHKSSSQGQDRHMIRMRQ | 1.257 | 1794 |
| | A112P | LIDIVDQLKNYVNDLVPEFLPAPEDVETNCEWSAFSCFQDA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_6408 | H35F, V98E | MRSSPGNMERIVICLMVIFLGTLVHKSSSQGQDREMIRMRQ | 1.258 | 1795 |
| | | LIDIVDQLKNYVNDLVPEFLPAPEDVETNCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_4962 | T110D, R114Q, | MRSSPGNMERIVICLMVIFLGTLVHKSSSQGQDRHMIRMRQ | 1.258 | 1796 |
| | H118D, R119T | LIDIVDQLKNYVNDLVPEFLPAPEDVETNCEWSAFSCFQKA | | |
| | | QLKSANTGNNERIINVSIKKLKRKPPSDNAGQRQKDTLTCP | | |
| | | SCDSYEKKPPKEFLERFKSLLQKMIHQHLSSRTHGSEDS | | |
| IL-21_4990 | P107V, R115G, | MRSSPGNMERIVICLMVIFLGTLVHKSSSQGQDRHMIRMRQ | 1.261 | 1797 |
| | H118D, T121E | LIDIVDQLKNYVNDLVPEFLPAPEDVETNCEWSAFSCFQKA | | |
| | | QLKSANTGNNERIINVSIKKLKRKVPSTNAGRGQKDRLECP | | |
| | | SCDSYEKKPPKEFLERFKSLLQKMIHQHLSSRTHGSEDS | | |
| IL-21_6134 | H35I, V98E, | MRSSPGNMERIVICLMVIFLGTLVHKSSSQGQDRIMIRMRQ | 1.262 | 1798 |
| | T110D, A112P | LIDIVDQLKNYVNDLVPEFLPAPEDVETNCEWSAFSCFQKA | | |
| | | QLKSANTGNNERIINESIKKLKRKPPSDNPGRRQKHRLTCP | | |
| | | SCDSYEKKPPKEFLERFKSLLQKMIHQHLSSRTHGSEDS | | |
| IL-21_5524 | R114G, R115D, | MRSSPGNMERIVICLMVIFLGTLVHKSSSQGQDRHMIRMRQ | 1.263 | 1799 |
| | H118D | LIDIVDQLKNYVNDLVPEFLPAPEDVETNCEWSAFSCFQKA | | |
| | | QLKSANTGNNERIINVSIKKLKRKPPSTNAGGDQKDRLTCP | | |
| | | SCDSYEKKPPKEFLERFKSLLQKMIHQHLSSRTHGSEDS | | |
| IL-21_5746 | N70G, P107V, | MRSSPGNMERIVICLMVIFLGTLVHKSSSQGQDRHMIRMRQ | 1.264 | 1800 |
| | R115D | LIDIVDQLKNYVNDLVPEFLPAPEDVETGCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5328 | N70G, R114Q, | MRSSPGNMERIVICLMVIFLGTLVHKSSSQGQDRHMIRMRQ | 1.264 | 1801 |
| | R115G, T121E | LIDIVDQLKNYVNDLVPEFLPAPEDVETGCEWSAFSCFQKA | | |
| | | QLKSANTGNNERIINVSIKKLKRKPPSTNAGQGQKHRLECP | | |
| | | SCDSYEKKPPKEFLERFKSLLQKMIHQHLSSRTHGSEDS | | |
| IL-21_5646 | V98E, P107V, | MRSSPGNMERIVICLMVIFLGTLVHKSSSQGQDRHMIRMRQ | 1.265 | 1802 |
| | T121E | LIDIVDQLKNYVNDLVPEFLPAPEDVETNCEWSAFSCFQKA | | |
| | | QLKSANTGNNERIINESIKKLKRKVPSTNAGRRQKHRLECP | | |
| | | SCDSYEKKPPKEFLERFKSLLQKMIHQHLSSRTHGSEDS | | |
| IL-21_4947 | T110D, R115G, | MRSSPGNMERIVICLMVIFLGTLVHKSSSQGQDRHMIRMRQ | 1.265 | 1803 |
| | H118D, R119T | LIDIVDQLKNYVNDLVPEFLPAPEDVETNCEWSAFSCFQKA | | |
| | | QLKSANTGNNERIINVSIKKLKRKPPSDNAGRGQKDTLTCP | | |
| | | SCDSYEKKPPKEFLERFKSLLQKMIHQHLSSRTHGSEDS | | |
| IL-21_5561 | T110D, R115D, | MRSSPGNMERIVICLMVIFLGTLVHKSSSQGQDRHMIRMRQ | 1.267 | 1804 |
| | H118D | LIDIVDQLKNYVNDLVPEFLPAPEDVETNCEWSAFSCFQKA | | |
| | | QLKSANTGNNERIINVSIKKLKRKPPSDNAGRDQKDRLTCP | | |
| | | SCDSYEKKPPKEFLERFKSLLQKMIHQHLSSRTHGSEDS | | |
| IL-21_4954 | T110D, R114G, | MRSSPGNMERIVICLMVIFLGTLVHKSSSQGQDRHMIRMRQ | 1.268 | 1805 |
| | R115G, T121E | LIDIVDQLKNYVNDLVPEFLPAPEDVETNCEWSAFSCFQKA | | |
| | | QLKSANTGNNERIINVSIKKLKRKPPSDNAGGGQKHRLECP | | |
| | | SCDSYEKKPPKEFLERFKSLLQKMIHQHLSSRTHGSEDS | | |
| IL-21_5907 | I45T, K81D, | MRSSPGNMERIVICLMVIFLGTLVHKSSSQGQDRHMIRMRQ | 1.27 | 1806 |
| | P107V, A112P | LIDTVDQLKNYVNDLVPEFLPAPEDVETNCEWSAFSCFQDA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_6296 | H35F, T110D, | MRSSPGNMERIVICLMVIFLGTLVHKSSSQGQDREMIRMRQ | 1.271 | 1807 |
| | R114Q | LIDIVDQLKNYVNDLVPEFLPAPEDVETNCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_4913 | R114Q, R115G, | MRSSPGNMERIVICLMVIFLGTLVHKSSSQGQDRHMIRMRQ | 1.274 | 1808 |
| | R119T, T121E | LIDIVDQLKNYVNDLVPEFLPAPEDVETNCEWSAFSCFQKA | | |
| | | QLKSANTGNNERIINVSIKKLKRKPPSTNAGQGQKHTLECP | | |
| | | SCDSYEKKPPKEFLERFKSLLQKMIHQHLSSRTHGSEDS | | |
| IL-21_6295 | H35F, T110D, | MRSSPGNMERIVICLMVIFLGTLVHKSSSQGQDRFMIRMRQ | 1.274 | 1809 |
| | R114G | LIDIVDQLKNYVNDLVPEFLPAPEDVETNCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5714 | N70G, R115D, | MRSSPGNMERIVICLMVIFLGTLVHKSSSQGQDRHMIRMRQ | 1.275 | 1810 |
| | T121E | LIDIVDQLKNYVNDLVPEFLPAPEDVETGCEWSAFSCFQKA | | |
| | | QLKSANTGNNERIINVSIKKLKRKPPSTNAGRDQKHRLECP | | |
| | | SCDSYEKKPPKEFLERFKSLLQKMIHQHLSSRTHGSEDS | | |
| IL-21_5317 | N70G, R114G, | MRSSPGNMERIVICLMVIFLGTLVHKSSSQGQDRHMIRMRQ | 1.275 | 1811 |
| | H118D, T121E | LIDIVDQLKNYVNDLVPEFLPAPEDVETGCEWSAFSCFQKA | | |
| | | QLKSANTGNNERIINVSIKKLKRKPPSTNAGGRQKDRLECP | | |
| | | SCDSYEKKPPKEFLERFKSLLQKMIHQHLSSRTHGSEDS | | |
| IL-21_6201 | H35I, I45T, | MRSSPGNMERIVICLMVIFLGTLVHKSSSQGQDRIMIRMRQ | 1.277 | 1812 |
| | R114G, R115D | LIDTVDQLKNYVNDLVPEFLPAPEDVETNCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5376 | N70G, T110D, | MRSSPGNMERIVICLMVIFLGTLVHKSSSQGQDRHMIRMRQ | 1.278 | 1813 |
| | A112P, R115D | LIDIVDQLKNYVNDLVPEFLPAPEDVETGCEWSAFSCFQKA | | |
| | | QLKSANTGNNERIINVSIKKLKRKPPSDNPGRDQKHRLTCP | | |
| | | SCDSYEKKPPKEFLERFKSLLQKMIHQHLSSRTHGSEDS | | |
| IL-21_4955 | T110D, R114G, | MRSSPGNMERIVICLMVIFLGTLVHKSSSQGQDRHMIRMRQ | 1.278 | 1814 |
| | R115G, R119T | LIDIVDQLKNYVNDLVPEFLPAPEDVETNCEWSAFSCFQKA | | |
| | | QLKSANTGNNERIINVSIKKLKRKPPSDNAGGGQKHTLTCP | | |
| | | SCDSYEKKPPKEFLERFKSLLQKMIHQHLSSRTHGSEDS | | |
| IL-21_4956 | T110D, R114G, | MRSSPGNMERIVICLMVIFLGTLVHKSSSQGQDRHMIRMRQ | 1.28 | 1815 |
| | R115G, H118D | LIDIVDQLKNYVNDLVPEFLPAPEDVETNCEWSAFSCFQKA | | |
| | | QLKSANTGNNERIINVSIKKLKRKPPSDNAGGGQKDRLTCP | | |
| | | SCDSYEKKPPKEFLERFKSLLQKMIHQHLSSRTHGSEDS | | |
| IL-21_4907 | R114G, R115G, | MRSSPGNMERIVICLMVIFLGTLVHKSSSQGQDRHMIRMRQ | 1.281 | 1816 |
| | H118D, T121E | LIDIVDQLKNYVNDLVPEFLPAPEDVETNCEWSAFSCFQKA | | |
| | | QLKSANTGNNERIINVSIKKLKRKPPSTNAGGGQKDRLECP | | |
| | | SCDSYEKKPPKEFLERFKSLLQKMIHQHLSSRTHGSEDS | | |
| IL-21_5715 | N70G, R115D, | MRSSPGNMERIVICLMVIFLGTLVHKSSSQGQDRHMIRMRQ | 1.282 | 1817 |
| | R119T | LIDIVDQLKNYVNDLVPEFLPAPEDVETGCEWSAFSCFQKA | | |
| | | QLKSANTGNNERIINVSIKKLKRKPPSTNAGRDQKHTLTCP | | |
| | | SCDSYEKKPPKEFLERFKSLLQKMIHQHLSSRTHGSEDS | | |
| IL-21_6275 | I45T, K81D, | MRSSPGNMERIVICLMVIFLGTLVHKSSSQGQDRHMIRMRQ | 1.283 | 1818 |
| | P107V | LIDTVDQLKNYVNDLVPEFLPAPEDVETNCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5768 | N70G, K81D, | MRSSPGNMERIVICLMVIFLGTLVHKSSSQGQDRHMIRMRQ | 1.285 | 1819 |
| | A112P | LIDIVDQLKNYVNDLVPEFLPAPEDVETGCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5523 | R114G, R115D, | MRSSPGNMERIVICLMVIFLGTLVHKSSSQGQDRHMIRMRQ | 1.286 | 1820 |
| | R119T | LIDIVDQLKNYVNDLVPEFLPAPEDVETNCEWSAFSCFQKA | | |
| | | QLKSANTGNNERIINVSIKKLKRKPPSTNAGGDQKHTLTCP | | |
| | | SCDSYEKKPPKEFLERFKSLLQKMIHQHLSSRTHGSEDS | | |
| IL-21_6101 | H35I, T110D, | MRSSPGNMERIVICLMVIFLGTLVHKSSSQGQDRIMIRMRQ | 1.289 | 1821 |
| | R114G, R115G | LIDIVDQLKNYVNDLVPEFLPAPEDVETNCEWSAFSCFQKA | | |
| | | QLKSANTGNNERIINVSIKKLKRKPPSDNAGGGQKHRLTCP | | |
| | | SCDSYEKKPPKEFLERFKSLLQKMIHQHLSSRTHGSEDS | | |
| IL-21_5595 | P107V, R114Q, | MRSSPGNMERIVICLMVIFLGTLVHKSSSQGQDRHMIRMRQ | 1.291 | 1822 |
| | H118D | LIDIVDQLKNYVNDLVPEFLPAPEDVETNCEWSAFSCFQKA | | |
| | | QLKSANTGNNERIINVSIKKLKRKVPSTNAGQRQKDRLTCP | | |
| | | SCDSYEKKPPKEFLERFKSLLQKMIHQHLSSRTHGSEDS | | |
| IL-21_4934 | A112P, R114G, | MRSSPGNMERIVICLMVIFLGTLVHKSSSQGQDRHMIRMRQ | 1.294 | 1823 |
| | R115D, H118D | LIDIVDQLKNYVNDLVPEFLPAPEDVETNCEWSAFSCFQKA | | |
| | | QLKSANTGNNERIINVSIKKLKRKPPSTNPGGDQKDRLTCP | | |
| | | SCDSYEKKPPKEFLERFKSLLQKMIHQHLSSRTHGSEDS | | |
| IL-21_5533 | R114Q, R115D, | MRSSPGNMERIVICLMVIFLGTLVHKSSSQGQDRHMIRMRQ | 1.295 | 1824 |
| | H118D | LIDIVDQLKNYVNDLVPEFLPAPEDVETNCEWSAFSCFQKA | | |
| | | QLKSANTGNNERIINVSIKKLKRKPPSTNAGQDQKDRLTCP | | |
| | | SCDSYEKKPPKEFLERFKSLLQKMIHQHLSSRTHGSEDS | | |
| IL-21_5407 | N70G, P107V, | MRSSPGNMERIVICLMVIFLGTLVHKSSSQGQDRHMIRMRQ | 1.296 | 1825 |
| | T110D, H118D | LIDIVDQLKNYVNDLVPEFLPAPEDVETGCEWSAFSCFQKA | | |
| | | QLKSANTGNNERIINVSIKKLKRKVPSDNAGRRQKDRLTCP | | |
| | | SCDSYEKKPPKEFLERFKSLLQKMIHQHLSSRTHGSEDS | | |
| IL-21_6267 | I45T, V98E, | MRSSPGNMERIVICLMVIFLGTLVHKSSSQGQDRHMIRMRQ | 1.298 | 1826 |
| | T110D | LIDTVDQLKNYVNDLVPEFLPAPEDVETNCEWSAFSCFQKA | | |
| | | QLKSANTGNNERIINESIKKLKRKPPSDNAGRRQKHRLTCP | | |
| | | SCDSYEKKPPKEFLERFKSLLQKMIHQHLSSRTHGSEDS | | |
| IL-21_5952 | H35F, A112P, | MRSSPGNMERIVICLMVIFLGTLVHKSSSQGQDRFMIRMRQ | 1.299 | 1827 |
| | R114Q, R115G | LIDIVDQLKNYVNDLVPEFLPAPEDVETNCEWSAFSCFQKA | | |
| | | QLKSANTGNNERIINVSIKKLKRKPPSTNPGQGQKHRLTCP | | |
| | | SCDSYEKKPPKEFLERFKSLLQKMIHQHLSSRTHGSEDS | | |
| IL-21_6089 | H35F, I45T, | MRSSPGNMERIVICLMVIFLGTLVHKSSSQGQDREMIRMRQ | 1.3 | 1828 |
| | N70G, R115D | LIDTVDQLKNYVNDLVPEFLPAPEDVETGCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5720 | N70G, R114G, | MRSSPGNMERIVICLMVIFLGTLVHKSSSQGQDRHMIRMRQ | 1.3 | 1829 |
| | R115G | LIDIVDQLKNYVNDLVPEFLPAPEDVETGCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5725 | N70G, R114Q, | MRSSPGNMERIVICLMVIFLGTLVHKSSSQGQDRHMIRMRQ | 1.301 | 1830 |
| | R115G | LIDIVDQLKNYVNDLVPEFLPAPEDVETGCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5889 | I45T, V98E, | MRSSPGNMERIVICLMVIFLGTLVHKSSSQGQDRHMIRMRQ | 1.301 | 1831 |
| | P107V, T110D | LIDTVDQLKNYVNDLVPEFLPAPEDVETNCEWSAFSCFQKA | | |
| | | QLKSANTGNNERIINESIKKLKRKVPSDNAGRRQKHRLTCP | | |
| | | SCDSYEKKPPKEFLERFKSLLQKMIHQHLSSRTHGSEDS | | |
| IL-21_5591 | P107V, R114G, | MRSSPGNMERIVICLMVIFLGTLVHKSSSQGQDRHMIRMRQ | 1.303 | 1832 |
| | R115G | LIDIVDQLKNYVNDLVPEFLPAPEDVETNCEWSAFSCFQKA | | |
| | | QLKSANTGNNERIINVSIKKLKRKVPSTNAGGGQKHRLTCP | | |
| | | SCDSYEKKPPKEFLERFKSLLQKMIHQHLSSRTHGSEDS | | |
| IL-21_4998 | P107V, R114G, | MRSSPGNMERIVICLMVIFLGTLVHKSSSQGQDRHMIRMRQ | 1.303 | 1833 |
| | R115G, T121E | LIDIVDQLKNYVNDLVPEFLPAPEDVETNCEWSAFSCFQKA | | |
| | | QLKSANTGNNERIINVSIKKLKRKVPSTNAGGGQKHRLECP | | |
| | | SCDSYEKKPPKEFLERFKSLLQKMIHQHLSSRTHGSEDS | | |
| IL-21_5015 | P107V, A112P, | MRSSPGNMERIVICLMVIFLGTLVHKSSSQGQDRHMIRMRQ | 1.304 | 1834 |
| | H118D, R119T | LIDIVDQLKNYVNDLVPEFLPAPEDVETNCEWSAFSCFQKA | | |
| | | QLKSANTGNNERIINVSIKKLKRKVPSTNPGRRQKDTLTCP | | |
| | | SCDSYEKKPPKEFLERFKSLLQKMIHQHLSSRTHGSEDS | | |
| IL-21_6098 | H35I, A112P, | MRSSPGNMERIVICLMVIFLGTLVHKSSSQGQDRIMIRMRQ | 1.304 | 1835 |
| | R114G, R115D | LIDIVDQLKNYVNDLVPEFLPAPEDVETNCEWSAFSCFQKA | | |
| | | QLKSANTGNNERIINVSIKKLKRKPPSTNPGGDQKHRLTCP | | |
| | | SCDSYEKKPPKEFLERFKSLLQKMIHQHLSSRTHGSEDS | | |
| IL-21_5801 | T110D, R115D | MRSSPGNMERIVICLMVIFLGTLVHKSSSQGQDRHMIRMRQ | 1.304 | 1836 |
| | | LIDIVDQLKNYVNDLVPEFLPAPEDVETNCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6326 | H35F, N70G, | MRSSPGNMERIVICLMVIFLGTLVHKSSSQGQDRFMIRMRQ | 1.304 | 1837 |
| | V98E | LIDIVDQLKNYVNDLVPEFLPAPEDVETGCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5335 | N70G, A112P, | MRSSPGNMERIVICLMVIFLGTLVHKSSSQGQDRHMIRMRQ | 1.305 | 1838 |
| | H118D, T121E | LIDIVDQLKNYVNDLVPEFLPAPEDVETGCEWSAFSCFQKA | | |
| | | QLKSANTGNNERIINVSIKKLKRKPPSTNPGRRQKDRLECP | | |
| | | SCDSYEKKPPKEFLERFKSLLQKMIHQHLSSRTHGSEDS | | |
| IL-21_6102 | H35I, T110D, | MRSSPGNMERIVICLMVIFLGTLVHKSSSQGQDRIMIRMRQ | 1.305 | 1839 |
| | R114G, R115D | LIDIVDQLKNYVNDLVPEFLPAPEDVETNCEWSAFSCFQKA | | |
| | | QLKSANTGNNERIINVSIKKLKRKPPSDNAGGDQKHRLTCP | | |
| | | SCDSYEKKPPKEFLERFKSLLQKMIHQHLSSRTHGSEDS | | |
| IL-21_4923 | A112P, R115D, | MRSSPGNMERIVICLMVIFLGTLVHKSSSQGQDRHMIRMRQ | 1.306 | 1840 |
| | R119T, T121E | LIDIVDQLKNYVNDLVPEFLPAPEDVETNCEWSAFSCFQKA | | |
| | | QLKSANTGNNERIINVSIKKLKRKPPSTNPGRDQKHTLECP | | |
| | | SCDSYEKKPPKEFLERFKSLLQKMIHQHLSSRTHGSEDS | | |
| IL-21_6413 | H35I, R115D | MRSSPGNMERIVICLMVIFLGTLVHKSSSQGQDRIMIRMRQ | 1.306 | 1841 |
| | | LIDIVDQLKNYVNDLVPEFLPAPEDVETNCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5902 | I45T, K81D, | MRSSPGNMERIVICLMVIFLGTLVHKSSSQGQDRHMIRMRQ | 1.306 | 1842 |
| | T110D, A112P | LIDTVDQLKNYVNDLVPEFLPAPEDVETNCEWSAFSCFQDA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5731 | N70G, A112P, | MRSSPGNMERIVICLMVIFLGTLVHKSSSQGQDRHMIRMRQ | 1.307 | 1843 |
| | R115D | LIDIVDQLKNYVNDLVPEFLPAPEDVETGCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_6320 | H35F, N70G, | MRSSPGNMERIVICLMVIFLGTLVHKSSSQGQDRFMIRMRQ | 1.307 | 1844 |
| | R115D | LIDIVDQLKNYVNDLVPEFLPAPEDVETGCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_6271 | I45T, K81D, | MRSSPGNMERIVICLMVIFLGTLVHKSSSQGQDRHMIRMRQ | 1.308 | 1845 |
| | R114G | LIDTVDQLKNYVNDLVPEFLPAPEDVETNCEWSAFSCFQDA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5047 | P107V, T110D, | MRSSPGNMERIVICLMVIFLGTLVHKSSSQGQDRHMIRMRQ | 1.309 | 1846 |
| | R114Q, R119T | LIDIVDQLKNYVNDLVPEFLPAPEDVETNCEWSAFSCFQKA | | |
| | | QLKSANTGNNERIINVSIKKLKRKVPSDNAGQRQKHTLTCP | | |
| | | SCDSYEKKPPKEFLERFKSLLQKMIHQHLSSRTHGSEDS | | |
| IL-21_5861 | I45T, P107V, | MRSSPGNMERIVICLMVIFLGTLVHKSSSQGQDRHMIRMRQ | 1.309 | 1847 |
| | R114Q, R115D | LIDTVDQLKNYVNDLVPEFLPAPEDVETNCEWSAFSCFQKA | | |
| | | QLKSANTGNNERIINVSIKKLKRKVPSTNAGQDQKHRLTCP | | |
| | | SCDSYEKKPPKEFLERFKSLLQKMIHQHLSSRTHGSEDS | | |
| IL-21_5310 | N70G, R115G, | MRSSPGNMERIVICLMVIFLGTLVHKSSSQGQDRHMIRMRQ | 1.311 | 1848 |
| | R119T, T121E | LIDIVDQLKNYVNDLVPEFLPAPEDVETGCEWSAFSCFQKA | | |
| | | QLKSANTGNNERIINVSIKKLKRKPPSTNAGRGQKHTLECP | | |
| | | SCDSYEKKPPKEFLERFKSLLQKMIHQHLSSRTHGSEDS | | |
| IL-21_5530 | R114Q, R115G, | MRSSPGNMERIVICLMVIFLGTLVHKSSSQGQDRHMIRMRQ | 1.311 | 1849 |
| | H118D | LIDIVDQLKNYVNDLVPEFLPAPEDVETNCEWSAFSCFQKA | | |
| | | QLKSANTGNNERIINVSIKKLKRKPPSTNAGQGQKDRLTCP | | |
| | | SCDSYEKKPPKEFLERFKSLLQKMIHQHLSSRTHGSEDS | | |
| IL-21_6106 | H35I, T110D, | MRSSPGNMERIVICLMVIFLGTLVHKSSSQGQDRIMIRMRQ | 1.313 | 1850 |
| | A112P, R115D | LIDIVDQLKNYVNDLVPEFLPAPEDVETNCEWSAFSCFQKA | | |
| | | QLKSANTGNNERIINVSIKKLKRKPPSDNPGRDQKHRLTCP | | |
| | | SCDSYEKKPPKEFLERFKSLLQKMIHQHLSSRTHGSEDS | | |
| IL-21_4964 | T110D, R114Q, | MRSSPGNMERIVICLMVIFLGTLVHKSSSQGQDRHMIRMRQ | 1.313 | 1851 |
| | R115G, R119T | LIDIVDQLKNYVNDLVPEFLPAPEDVETNCEWSAFSCFQKA | | |
| | | QLKSANTGNNERIINVSIKKLKRKPPSDNAGQGQKHTLTCP | | |
| | | SCDSYEKKPPKEFLERFKSLLQKMIHQHLSSRTHGSEDS | | |
| IL-21_5402 | N70G, P107V, | MRSSPGNMERIVICLMVIFLGTLVHKSSSQGQDRHMIRMRQ | 1.315 | 1852 |
| | A112P, R115D | LIDIVDQLKNYVNDLVPEFLPAPEDVETGCEWSAFSCFQKA | | |
| | | QLKSANTGNNERIINVSIKKLKRKVPSTNPGRDQKHRLTCP | | |
| | | SCDSYEKKPPKEFLERFKSLLQKMIHQHLSSRTHGSEDS | | |
| IL-21_6284 | I45T, N70G, | MRSSPGNMERIVICLMVIFLGTLVHKSSSQGQDRHMIRMRQ | 1.319 | 1853 |
| | K81D | LIDTVDQLKNYVNDLVPEFLPAPEDVETGCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6336 | H35F, I45T, | MRSSPGNMERIVICLMVIFLGTLVHKSSSQGQDREMIRMRQ | 1.32 | 1854 |
| | K81D | LIDTVDQLKNYVNDLVPEFLPAPEDVETNCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5339 | N70G, A112P, | MRSSPGNMERIVICLMVIFLGTLVHKSSSQGQDRHMIRMRQ | 1.32 | 1855 |
| | R115G, H118D | LIDIVDQLKNYVNDLVPEFLPAPEDVETGCEWSAFSCFQKA | | |
| | | QLKSANTGNNERIINVSIKKLKRKPPSTNPGRGQKDRLTCP | | |
| | | SCDSYEKKPPKEFLERFKSLLQKMIHQHLSSRTHGSEDS | | |
| IL-21_5758 | N70G, V98E, | MRSSPGNMERIVICLMVIFLGTLVHKSSSQGQDRHMIRMRQ | 1.321 | 1856 |
| | A112P | LIDIVDQLKNYVNDLVPEFLPAPEDVETGCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5815 | V98E, R119T | MRSSPGNMERIVICLMVIFLGTLVHKSSSQGQDRHMIRMRQ | 1.324 | 1857 |
| | | LIDIVDQLKNYVNDLVPEFLPAPEDVETNCEWSAFSCFQKA | | |
| | | QLKSANTGNNERIINESIKKLKRKPPSTNAGRRQKHTLTCP | | |
| | | SCDSYEKKPPKEFLERFKSLLQKMIHQHLSSRTHGSEDS | | |
| IL-21_4987 | T110D, A112P, | MRSSPGNMERIVICLMVIFLGTLVHKSSSQGQDRHMIRMRQ | 1.324 | 1858 |
| | R114Q, R115D | LIDIVDQLKNYVNDLVPEFLPAPEDVETNCEWSAFSCFQKA | | |
| | | QLKSANTGNNERIINVSIKKLKRKPPSDNPGQDQKHRLTCP | | |
| | | SCDSYEKKPPKEFLERFKSLLQKMIHQHLSSRTHGSEDS | | |
| IL-21_6274 | I45T, K81D, | MRSSPGNMERIVICLMVIFLGTLVHKSSSQGQDRHMIRMRQ | 1.324 | 1859 |
| | T110D | LIDTVDQLKNYVNDLVPEFLPAPEDVETNCEWSAFSCFQDA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5737 | N70G, T110D, | MRSSPGNMERIVICLMVIFLGTLVHKSSSQGQDRHMIRMRQ | 1.324 | 1860 |
| | R115G | LIDIVDQLKNYVNDLVPEFLPAPEDVETGCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_6172 | H35I, N70G, | MRSSPGNMERIVICLMVIFLGTLVHKSSSQGQDRIMIRMRQ | 1.324 | 1861 |
| | A112P, R115D | LIDIVDQLKNYVNDLVPEFLPAPEDVETGCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5362 | N70G, T110D, | MRSSPGNMERIVICLMVIFLGTLVHKSSSQGQDRHMIRMRQ | 1.327 | 1862 |
| | R114G, T121E | LIDIVDQLKNYVNDLVPEFLPAPEDVETGCEWSAFSCFQKA | | |
| | | QLKSANTGNNERIINVSIKKLKRKPPSDNAGGRQKHRLECP | | |
| | | SCDSYEKKPPKEFLERFKSLLQKMIHQHLSSRTHGSEDS | | |
| IL-21_5374 | N70G, T110D, | MRSSPGNMERIVICLMVIFLGTLVHKSSSQGQDRHMIRMRQ | 1.327 | 1863 |
| | A112P, H118D | LIDIVDQLKNYVNDLVPEFLPAPEDVETGCEWSAFSCFQKA | | |
| | | QLKSANTGNNERIINVSIKKLKRKPPSDNPGRRQKDRLTCP | | |
| | | SCDSYEKKPPKEFLERFKSLLQKMIHQHLSSRTHGSEDS | | |
| IL-21_5631 | V98E, A112P, | MRSSPGNMERIVICLMVIFLGTLVHKSSSQGQDRHMIRMRQ | 1.328 | 1864 |
| | T121E | LIDIVDQLKNYVNDLVPEFLPAPEDVETNCEWSAFSCFQKA | | |
| | | QLKSANTGNNERIINESIKKLKRKPPSTNPGRRQKHRLECP | | |
| | | SCDSYEKKPPKEFLERFKSLLQKMIHQHLSSRTHGSEDS | | |
| IL-21_6202 | H35I, I45T, | MRSSPGNMERIVICLMVIFLGTLVHKSSSQGQDRIMIRMRQ | 1.328 | 1865 |
| | R114Q, R115G | LIDTVDQLKNYVNDLVPEFLPAPEDVETNCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5408 | N70G, P107V, | MRSSPGNMERIVICLMVIFLGTLVHKSSSQGQDRHMIRMRQ | 1.328 | 1866 |
| | T110D, R115G | LIDIVDQLKNYVNDLVPEFLPAPEDVETGCEWSAFSCFQKA | | |
| | | QLKSANTGNNERIINVSIKKLKRKVPSDNAGRGQKHRLTCP | | |
| | | SCDSYEKKPPKEFLERFKSLLQKMIHQHLSSRTHGSEDS | | |
| IL-21_6287 | H35F, R114Q, | MRSSPGNMERIVICLMVIFLGTLVHKSSSQGQDRFMIRMRQ | 1.329 | 1867 |
| | R115G | LIDIVDQLKNYVNDLVPEFLPAPEDVETNCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5313 | N70G, R115D, | MRSSPGNMERIVICLMVIFLGTLVHKSSSQGQDRHMIRMRQ | 1.333 | 1868 |
| | R119T, T121E | LIDIVDQLKNYVNDLVPEFLPAPEDVETGCEWSAFSCFQKA | | |
| | | QLKSANTGNNERIINVSIKKLKRKPPSTNAGRDQKHTLECP | | |
| | | SCDSYEKKPPKEFLERFKSLLQKMIHQHLSSRTHGSEDS | | |
| IL-21_6095 | H35F, I45T, | MRSSPGNMERIVICLMVIFLGTLVHKSSSQGQDREMIRMRQ | 1.334 | 1869 |
| | N70G, V98E | LIDTVDQLKNYVNDLVPEFLPAPEDVETGCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5019 | P107V, A112P, | MRSSPGNMERIVICLMVIFLGTLVHKSSSQGQDRHMIRMRQ | 1.334 | 1870 |
| | R115D, T121E | LIDIVDQLKNYVNDLVPEFLPAPEDVETNCEWSAFSCFQKA | | |
| | | QLKSANTGNNERIINVSIKKLKRKVPSTNPGRDQKHRLECP | | |
| | | SCDSYEKKPPKEFLERFKSLLQKMIHQHLSSRTHGSEDS | | |
| IL-21_6361 | H35I, V98E, | MRSSPGNMERIVICLMVIFLGTLVHKSSSQGQDRIMIRMRQ | 1.335 | 1871 |
| | A112P | LIDIVDQLKNYVNDLVPEFLPAPEDVETNCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5884 | I45T, V98E, | MRSSPGNMERIVICLMVIFLGTLVHKSSSQGQDRHMIRMRQ | 1.336 | 1872 |
| | P017V, R115G | LIDTVDQLKNYVNDLVPEFLPAPEDVETNCEWSAFSCFQKA | | |
| | | QLKSANTGNNERIINESIKKLKRKVPSTNAGRGQKHRLTCP | | |
| | | SCDSYEKKPPKEFLERFKSLLQKMIHQHLSSRTHGSEDS | | |
| IL-21_6293 | H35F, T110D, | MRSSPGNMERIVICLMVIFLGTLVHKSSSQGQDREMIRMRQ | 1.336 | 1873 |
| | R115G | LIDIVDQLKNYVNDLVPEFLPAPEDVETNCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_5319 | N70G, R114G, | MRSSPGNMERIVICLMVIFLGTLVHKSSSQGQDRHMIRMRQ | 1.337 | 1874 |
| | R115G, T121E | LIDIVDQLKNYVNDLVPEFLPAPEDVETGCEWSAFSCFQKA | | |
| | | QLKSANTGNNERIINVSIKKLKRKPPSTNAGGGQKHRLECP | | |
| | | SCDSYEKKPPKEFLERFKSLLQKMIHQHLSSRTHGSEDS | | |
| IL-21_4995 | P107V, R114G, | MRSSPGNMERIVICLMVIFLGTLVHKSSSQGQDRHMIRMRQ | 1.337 | 1875 |
| | R119T, T121E | LIDIVDQLKNYVNDLVPEFLPAPEDVETNCEWSAFSCFQKA | | |
| | | QLKSANTGNNERIINVSIKKLKRKVPSTNAGGRQKHTLECP | | |
| | | SCDSYEKKPPKEFLERFKSLLQKMIHQHLSSRTHGSEDS | | |
| IL-21_5513 | R115D, R119T, | MRSSPGNMERIVICLMVIFLGTLVHKSSSQGQDRHMIRMRQ | 1.338 | 1876 |
| | T121E | LIDIVDQLKNYVNDLVPEFLPAPEDVETNCEWSAFSCFQKA | | |
| | | QLKSANTGNNERIINVSIKKLKRKPPSTNAGRDQKHTLECP | | |
| | | SCDSYEKKPPKEFLERFKSLLQKMIHQHLSSRTHGSEDS | | |
| IL-21_4940 | A112P, R114Q, | MRSSPGNMERIVICLMVIFLGTLVHKSSSQGQDRHMIRMRQ | 1.34 | 1877 |
| | R115G, H118D | LIDIVDQLKNYVNDLVPEFLPAPEDVETNCEWSAFSCFQKA | | |
| | | QLKSANTGNNERIINVSIKKLKRKPPSTNPGQGQKDRLTCP | | |
| | | SCDSYEKKPPKEFLERFKSLLQKMIHQHLSSRTHGSEDS | | |
| IL-21_4951 | T110D, R114G, | MRSSPGNMERIVICLMVIFLGTLVHKSSSQGQDRHMIRMRQ | 1.341 | 1878 |
| | R119T, T121E | LIDIVDQLKNYVNDLVPEFLPAPEDVETNCEWSAFSCFQKA | | |
| | | QLKSANTGNNERIINVSIKKLKRKPPSDNAGGRQKHTLECP | | |
| | | SCDSYEKKPPKEFLERFKSLLQKMIHQHLSSRTHGSEDS | | |
| IL-21_6246 | I45T, R114Q, | MRSSPGNMERIVICLMVIFLGTLVHKSSSQGQDRHMIRMRQ | 1.343 | 1879 |
| | R115D | LIDTVDQLKNYVNDLVPEFLPAPEDVETNCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_5389 | N70G, P107V, | MRSSPGNMERIVICLMVIFLGTLVHKSSSQGQDRHMIRMRQ | 1.346 | 1880 |
| | R114G, R119T | LIDIVDQLKNYVNDLVPEFLPAPEDVETGCEWSAFSCFQKA | | |
| | | QLKSANTGNNERIINVSIKKLKRKVPSTNAGGRQKHTLTCP | | |
| | | SCDSYEKKPPKEFLERFKSLLQKMIHQHLSSRTHGSEDS | | |
| IL-21_5869 | I45T, P107V, | MRSSPGNMERIVICLMVIFLGTLVHKSSSQGQDRHMIRMRQ | 1.346 | 1881 |
| | T110D, R114Q | LIDTVDQLKNYVNDLVPEFLPAPEDVETNCEWSAFSCFQKA | | |
| | | QLKSANTGNNERIINVSIKKLKRKVPSDNAGQRQKHRLTCP | | |
| | | SCDSYEKKPPKEFLERFKSLLQKMIHQHLSSRTHGSEDS | | |
| IL-21_5316 | N70G, R114G, | MRSSPGNMERIVICLMVIFLGTLVHKSSSQGQDRHMIRMRQ | 1.346 | 1882 |
| | R119T, T121E | LIDIVDQLKNYVNDLVPEFLPAPEDVETGCEWSAFSCFQKA | | |
| | | QLKSANTGNNERIINVSIKKLKRKPPSTNAGGRQKHTLECP | | |
| | | SCDSYEKKPPKEFLERFKSLLQKMIHQHLSSRTHGSEDS | | |
| IL-21_5367 | N70G, T110D, | MRSSPGNMERIVICLMVIFLGTLVHKSSSQGQDRHMIRMRQ | 1.347 | 1883 |
| | R114Q, T121E | LIDIVDQLKNYVNDLVPEFLPAPEDVETGCEWSAFSCFQKA | | |
| | | QLKSANTGNNERIINVSIKKLKRKPPSDNAGQRQKHRLECP | | |
| | | SCDSYEKKPPKEFLERFKSLLQKMIHQHLSSRTHGSEDS | | |
| IL-21_5585 | P107V, R115D, | MRSSPGNMERIVICLMVIFLGTLVHKSSSQGQDRHMIRMRQ | 1.349 | 1884 |
| | T121E | LIDIVDQLKNYVNDLVPEFLPAPEDVETNCEWSAFSCFQKA | | |
| | | QLKSANTGNNERIINVSIKKLKRKVPSTNAGRDQKHRLECP | | |
| | | SCDSYEKKPPKEFLERFKSLLQKMIHQHLSSRTHGSEDS | | |
| IL-21_5935 | I45T, N70G, | MRSSPGNMERIVICLMVIFLGTLVHKSSSQGQDRHMIRMRQ | 1.349 | 1885 |
| | V98E, R115G | LIDTVDQLKNYVNDLVPEFLPAPEDVETGCEWSAFSCFQKA | | |
| | | QLKSANTGNNERIINESIKKLKRKPPSTNAGRGQKHRLTCP | | |
| | | SCDSYEKKPPKEFLERFKSLLQKMIHQHLSSRTHGSEDS | | |
| IL-21_4985 | T110D, A112P | MRSSPGNMERIVICLMVIFLGTLVHKSSSQGQDRHMIRMRQ | 1.352 | 1886 |
| | R114Q, H118D | LIDIVDQLKNYVNDLVPEFLPAPEDVETNCEWSAFSCFQKA | | |
| | | QLKSANTGNNERIINVSIKKLKRKPPSDNPGQRQKDRLTCP | | |
| | | SCDSYEKKPPKEFLERFKSLLQKMIHQHLSSRTHGSEDS | | |
| IL-21_5385 | N70G, P107V, | MRSSPGNMERIVICLMVIFLGTLVHKSSSQGQDRHMIRMRQ | 1.352 | 1887 |
| | R115D, T121E | LIDIVDQLKNYVNDLVPEFLPAPEDVETGCEWSAFSCFQKA | | |
| | | QLKSANTGNNERIINVSIKKLKRKVPSTNAGRDQKHRLECP | | |
| | | SCDSYEKKPPKEFLERFKSLLQKMIHQHLSSRTHGSEDS | | |
| IL-21_4945 | T110D, R115G, | MRSSPGNMERIVICLMVIFLGTLVHKSSSQGQDRHMIRMRQ | 1.357 | 1888 |
| | R119T, T121E | LIDIVDQLKNYVNDLVPEFLPAPEDVETNCEWSAFSCFQKA | | |
| | | QLKSANTGNNERIINVSIKKLKRKPPSDNAGRGQKHTLECP | | |
| | | SCDSYEKKPPKEFLERFKSLLQKMIHQHLSSRTHGSEDS | | |
| IL-21_6026 | H35F, N70G, | MRSSPGNMERIVICLMVIFLGTLVHKSSSQGQDRFMIRMRQ | 1.357 | 1889 |
| | A112P, R114G | LIDIVDQLKNYVNDLVPEFLPAPEDVETGCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5325 | N70G, R114Q, | MRSSPGNMERIVICLMVIFLGTLVHKSSSQGQDRHMIRMRQ | 1.358 | 1890 |
| | R119T, T121E | LIDIVDQLKNYVNDLVPEFLPAPEDVETGCEWSAFSCFQKA | | |
| | | QLKSANTGNNERIINVSIKKLKRKPPSTNAGQRQKHTLECP | | |
| | | SCDSYEKKPPKEFLERFKSLLQKMIHQHLSSRTHGSEDS | | |
| IL-21_5393 | N70G, P107V, | MRSSPGNMERIVICLMVIFLGTLVHKSSSQGQDRHMIRMRQ | 1.358 | 1891 |
| | R114Q, T121E | LIDIVDQLKNYVNDLVPEFLPAPEDVETGCEWSAFSCFQKA | | |
| | | QLKSANTGNNERIINVSIKKLKRKVPSTNAGQRQKHRLECP | | |
| | | SCDSYEKKPPKEFLERFKSLLQKMIHQHLSSRTHGSEDS | | |
| IL-21_5950 | H35F, A112P, | MRSSPGNMERIVICLMVIFLGTLVHKSSSQGQDREMIRMRQ | 1.358 | 1892 |
| | R114G, R115G | LIDIVDQLKNYVNDLVPEFLPAPEDVETNCEWSAFSCFQKA | | |
| | | QLKSANTGNNERIINVSIKKLKRKPPSTNPGGGQKHRLTCP | | |
| | | SCDSYEKKPPKEFLERFKSLLQKMIHQHLSSRTHGSEDS | | |
| IL-21_5844 | N70G, V98E | MRSSPGNMERIVICLMVIFLGTLVHKSSSQGQDRHMIRMRQ | 1.358 | 1893 |
| | | LIDIVDQLKNYVNDLVPEFLPAPEDVETGCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5566 | T110D, R114G, | MRSSPGNMERIVICLMVIFLGTLVHKSSSQGQDRHMIRMRQ | 1.359 | 1894 |
| | R115D | LIDIVDQLKNYVNDLVPEFLPAPEDVETNCEWSAFSCFQKA | | |
| | | QLKSANTGNNERIINVSIKKLKRKPPSDNAGGDQKHRLTCP | | |
| | | SCDSYEKKPPKEFLERFKSLLQKMIHQHLSSRTHGSEDS | | |
| IL-21_5383 | N70G, P107V, | MRSSPGNMERIVICLMVIFLGTLVHKSSSQGQDRHMIRMRQ | 1.359 | 1895 |
| | R115G, R119T | LIDIVDQLKNYVNDLVPEFLPAPEDVETGCEWSAFSCFQKA | | |
| | | QLKSANTGNNERIINVSIKKLKRKVPSTNAGRGQKHTLTCP | | |
| | | SCDSYEKKPPKEFLERFKSLLQKMIHQHLSSRTHGSEDS | | |
| IL-21_5824 | K81D, T121E | MRSSPGNMERIVICLMVIFLGTLVHKSSSQGQDRHMIRMRQ | 1.361 | 1896 |
| | | LIDIVDQLKNYVNDLVPEFLPAPEDVETNCEWSAFSCFQDA | | |
| | | QLKSANTGNNERIINVSIKKLKRKPPSTNAGRRQKHRLECP | | |
| | | SCDSYEKKPPKEFLERFKSLLQKMIHQHLSSRTHGSEDS | | |
| IL-21_5568 | T110D, R114Q, | MRSSPGNMERIVICLMVIFLGTLVHKSSSQGQDRHMIRMRQ | 1.362 | 1897 |
| | R119T | LIDIVDQLKNYVNDLVPEFLPAPEDVETNCEWSAFSCFQKA | | |
| | | QLKSANTGNNERIINVSIKKLKRKPPSDNAGQRQKHTLTCP | | |
| | | SCDSYEKKPPKEFLERFKSLLQKMIHQHLSSRTHGSEDS | | |
| IL-21_5023 | P107V, A112P, | MRSSPGNMERIVICLMVIFLGTLVHKSSSQGQDRHMIRMRQ | 1.364 | 1898 |
| | R114G, R119T | LIDIVDQLKNYVNDLVPEFLPAPEDVETNCEWSAFSCFQKA | | |
| | | QLKSANTGNNERIINVSIKKLKRKVPSTNPGGRQKHTLTCP | | |
| | | SCDSYEKKPPKEFLERFKSLLQKMIHQHLSSRTHGSEDS | | |
| IL-21_5560 | T110D, R115D, | MRSSPGNMERIVICLMVIFLGTLVHKSSSQGQDRHMIRMRQ | 1.365 | 1899 |
| | R119T | LIDIVDQLKNYVNDLVPEFLPAPEDVETNCEWSAFSCFQKA | | |
| | | QLKSANTGNNERIINVSIKKLKRKPPSDNAGRDQKHTLTCP | | |
| | | SCDSYEKKPPKEFLERFKSLLQKMIHQHLSSRTHGSEDS | | |
| IL-21_5378 | N70G, T110D, | MRSSPGNMERIVICLMVIFLGTLVHKSSSQGQDRHMIRMRQ | 1.366 | 1900 |
| | A112P, R114Q | LIDIVDQLKNYVNDLVPEFLPAPEDVETGCEWSAFSCFQKA | | |
| | | QLKSANTGNNERIINVSIKKLKRKPPSDNPGQRQKHRLTCP | | |
| | | SCDSYEKKPPKEFLERFKSLLQKMIHQHLSSRTHGSEDS | | |
| IL-21_5851 | I45T, T110D, | MRSSPGNMERIVICLMVIFLGTLVHKSSSQGQDRHMIRMRQ | 1.368 | 1901 |
| | R114G, R115D | LIDTVDQLKNYVNDLVPEFLPAPEDVETNCEWSAFSCFQKA | | |
| | | QLKSANTGNNERIINVSIKKLKRKPPSDNAGGDQKHRLTCP | | |
| | | SCDSYEKKPPKEFLERFKSLLQKMIHQHLSSRTHGSEDS | | |
| IL-21_6210 | H35I, I45T, | MRSSPGNMERIVICLMVIFLGTLVHKSSSQGQDRIMIRMRQ | 1.369 | 1902 |
| | T110D, R114G | LIDTVDQLKNYVNDLVPEFLPAPEDVETNCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5849 | I45T, A112P, | MRSSPGNMERIVICLMVIFLGTLVHKSSSQGQDRHMIRMRQ | 1.372 | 1903 |
| | R114Q, R115D | LIDTVDQLKNYVNDLVPEFLPAPEDVETNCEWSAFSCFQKA | | |
| | | QLKSANTGNNERIINVSIKKLKRKPPSTNPGQDQKHRLTCP | | |
| | | SCDSYEKKPPKEFLERFKSLLQKMIHQHLSSRTHGSEDS | | |
| IL-21_6420 | H35I, K81D | MRSSPGNMERIVICLMVIFLGTLVHKSSSQGQDRIMIRMRQ | 1.373 | 1904 |
| | | LIDIVDQLKNYVNDLVPEFLPAPEDVETNCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5609 | P107V, T110D, | MRSSPGNMERIVICLMVIFLGTLVHKSSSQGQDRHMIRMRQ | 1.374 | 1905 |
| | R114G | LIDIVDQLKNYVNDLVPEFLPAPEDVETNCEWSAFSCFQKA | | |
| | | QLKSANTGNNERIINVSIKKLKRKVPSDNAGGRQKHRLTCP | | |
| | | SCDSYEKKPPKEFLERFKSLLQKMIHQHLSSRTHGSEDS | | |
| IL-21_5356 | N70G, T110D, | MRSSPGNMERIVICLMVIFLGTLVHKSSSQGQDRHMIRMRQ | 1.376 | 1906 |
| | R115G, T121E | LIDIVDQLKNYVNDLVPEFLPAPEDVETGCEWSAFSCFQKA | | |
| | | QLKSANTGNNERIINVSIKKLKRKPPSDNAGRGQKHRLECP | | |
| | | SCDSYEKKPPKEFLERFKSLLQKMIHQHLSSRTHGSEDS | | |
| IL-21_6214 | H35I, I45T, | MRSSPGNMERIVICLMVIFLGTLVHKSSSQGQDRIMIRMRQ | 1.378 | 1907 |
| | P107V, R115D | LIDTVDQLKNYVNDLVPEFLPAPEDVETNCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5329 | N70G, R114Q, | MRSSPGNMERIVICLMVIFLGTLVHKSSSQGQDRHMIRMRQ | 1.378 | 1908 |
| | R115G, R119T | LIDIVDQLKNYVNDLVPEFLPAPEDVETGCEWSAFSCFQKA | | |
| | | QLKSANTGNNERIINVSIKKLKRKPPSTNAGQGQKHTLTCP | | |
| | | SCDSYEKKPPKEFLERFKSLLQKMIHQHLSSRTHGSEDS | | |
| IL-21_5569 | T110D, R114Q, | MRSSPGNMERIVICLMVIFLGTLVHKSSSQGQDRHMIRMRQ | 1.379 | 1909 |
| | H118D | LIDIVDQLKNYVNDLVPEFLPAPEDVETNCEWSAFSCFQKA | | |
| | | QLKSANTGNNERIINVSIKKLKRKPPSDNAGQRQKDRLTCP | | |
| | | SCDSYEKKPPKEFLERFKSLLQKMIHQHLSSRTHGSEDS | | |
| IL-21_5860 | I45T, P107V, | MRSSPGNMERIVICLMVIFLGTLVHKSSSQGQDRHMIRMRQ | 1.38 | 1910 |
| | R114Q, R115G | LIDTVDQLKNYVNDLVPEFLPAPEDVETNCEWSAFSCFQKA | | |
| | | QLKSANTGNNERIINVSIKKLKRKVPSTNAGQGQKHRLTCP | | |
| | | SCDSYEKKPPKEFLERFKSLLQKMIHQHLSSRTHGSEDS | | |
| IL-21_5855 | I45T, T110D, | MRSSPGNMERIVICLMVIFLGTLVHKSSSQGQDRHMIRMRQ | 1.38 | 1911 |
| | A112P, R115D | LIDTVDQLKNYVNDLVPEFLPAPEDVETNCEWSAFSCFQKA | | |
| | | QLKSANTGNNERIINVSIKKLKRKPPSDNPGRDQKHRLTCP | | |
| | | SCDSYEKKPPKEFLERFKSLLQKMIHQHLSSRTHGSEDS | | |
| IL-21_5596 | P107V, R114Q, | MRSSPGNMERIVICLMVIFLGTLVHKSSSQGQDRHMIRMRQ | 1.381 | 1912 |
| | R115G | LIDIVDQLKNYVNDLVPEFLPAPEDVETNCEWSAFSCFQKA | | |
| | | QLKSANTGNNERIINVSIKKLKRKVPSTNAGQGQKHRLTCP | | |
| | | SCDSYEKKPPKEFLERFKSLLQKMIHQHLSSRTHGSEDS | | |
| IL-21_6177 | H35I, N70G, | MRSSPGNMERIVICLMVIFLGTLVHKSSSQGQDRIMIRMRQ | 1.381 | 1913 |
| | T110D, R114G | LIDIVDQLKNYVNDLVPEFLPAPEDVETGCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5342 | N70G, A112P, | MRSSPGNMERIVICLMVIFLGTLVHKSSSQGQDRHMIRMRQ | 1.382 | 1914 |
| | R115D, H118D | LIDIVDQLKNYVNDLVPEFLPAPEDVETGCEWSAFSCFQKA | | |
| | | QLKSANTGNNERIINVSIKKLKRKPPSTNPGRDQKDRLTCP | | |
| | | SCDSYEKKPPKEFLERFKSLLQKMIHQHLSSRTHGSEDS | | |
| IL-21_6241 | H351, I45T, | MRSSPGNMERIVICLMVIFLGTLVHKSSSQGQDRIMIRMRQ | 1.382 | 1915 |
| | N70G, V98E | LIDTVDQLKNYVNDLVPEFLPAPEDVETGCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_4928 | A112P, R114G, | MRSSPGNMERIVICLMVIFLGTLVHKSSSQGQDRHMIRMRQ | 1.383 | 1916 |
| | H118D, R119T | LIDIVDQLKNYVNDLVPEFLPAPEDVETNCEWSAFSCFQKA | | |
| | | QLKSANTGNNERIINVSIKKLKRKPPSTNPGGRQKDTLTCP | | |
| | | SCDSYEKKPPKEFLERFKSLLQKMIHQHLSSRTHGSEDS | | |
| IL-21_5581 | P107V, H118D, | MRSSPGNMERIVICLMVIFLGTLVHKSSSQGQDRHMIRMRQ | 1.384 | 1917 |
| | R119T | LIDIVDQLKNYVNDLVPEFLPAPEDVETNCEWSAFSCFQKA | | |
| | | QLKSANTGNNERIINVSIKKLKRKVPSTNAGRRQKDTLTCP | | |
| | | SCDSYEKKPPKEFLERFKSLLQKMIHQHLSSRTHGSEDS | | |
| IL-21_6235 | H35I, I45T, | MRSSPGNMERIVICLMVIFLGTLVHKSSSQGQDRIMIRMRQ | 1.386 | 1918 |
| | N70G, R115D | LIDTVDQLKNYVNDLVPEFLPAPEDVETGCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_6062 | H35F, I45T, | MRSSPGNMERIVICLMVIFLGTLVHKSSSQGQDREMIRMRQ | 1.386 | 1919 |
| | T110D, R115G | LIDTVDQLKNYVNDLVPEFLPAPEDVETNCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_6099 | H35I, A112P, | MRSSPGNMERIVICLMVIFLGTLVHKSSSQGQDRIMIRMRQ | 1.388 | 1920 |
| | R114Q, R115G | LIDIVDQLKNYVNDLVPEFLPAPEDVETNCEWSAFSCFQKA | | |
| | | QLKSANTGNNERIINVSIKKLKRKPPSTNPGQGQKHRLTCP | | |
| | | SCDSYEKKPPKEFLERFKSLLQKMIHQHLSSRTHGSEDS | | |
| IL-21_6119 | H35I, P107V, | MRSSPGNMERIVICLMVIFLGTLVHKSSSQGQDRIMIRMRQ | 1.389 | 1921 |
| | T110D, R114G | LIDIVDQLKNYVNDLVPEFLPAPEDVETNCEWSAFSCFQKA | | |
| | | QLKSANTGNNERIINVSIKKLKRKVPSDNAGGRQKHRLTCP | | |
| | | SCDSYEKKPPKEFLERFKSLLQKMIHQHLSSRTHGSEDS | | |
| IL-21_6205 | H35I, I45T, | MRSSPGNMERIVICLMVIFLGTLVHKSSSQGQDRIMIRMRQ | 1.39 | 1922 |
| | A112P, R115D | LIDTVDQLKNYVNDLVPEFLPAPEDVETNCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5584 | P107V, R115G, | MRSSPGNMERIVICLMVIFLGTLVHKSSSQGQDRHMIRMRQ | 1.391 | 1923 |
| | H118D | LIDIVDQLKNYVNDLVPEFLPAPEDVETNCEWSAFSCFQKA | | |
| | | QLKSANTGNNERIINVSIKKLKRKVPSTNAGRGQKDRLTCP | | |
| | | SCDSYEKKPPKEFLERFKSLLQKMIHQHLSSRTHGSEDS | | |
| IL-21_5388 | N70G, P107V, | MRSSPGNMERIVICLMVIFLGTLVHKSSSQGQDRHMIRMRQ | 1.393 | 1924 |
| | R114G, T121E | LIDIVDQLKNYVNDLVPEFLPAPEDVETGCEWSAFSCFQKA | | |
| | | QLKSANTGNNERIINVSIKKLKRKVPSTNAGGRQKHRLECP | | |
| | | SCDSYEKKPPKEFLERFKSLLQKMIHQHLSSRTHGSEDS | | |
| IL-21_4941 | A112P, R114Q, | MRSSPGNMERIVICLMVIFLGTLVHKSSSQGQDRHMIRMRQ | 1.394 | 1925 |
| | R115D, T121E | LIDIVDQLKNYVNDLVPEFLPAPEDVETNCEWSAFSCFQKA | | |
| | | QLKSANTGNNERIINVSIKKLKRKPPSTNPGQDQKHRLECP | | |
| | | SCDSYEKKPPKEFLERFKSLLQKMIHQHLSSRTHGSEDS | | |
| IL-21_5029 | P107V, A112P, | MRSSPGNMERIVICLMVIFLGTLVHKSSSQGQDRHMIRMRQ | 1.395 | 1926 |
| | R114Q, H118D | LIDIVDQLKNYVNDLVPEFLPAPEDVETNCEWSAFSCFQKA | | |
| | | QLKSANTGNNERIINVSIKKLKRKVPSTNPGQRQKDRLTCP | | |
| | | SCDSYEKKPPKEFLERFKSLLQKMIHQHLSSRTHGSEDS | | |
| IL-21_4906 | R114G, R115G, | MRSSPGNMERIVICLMVIFLGTLVHKSSSQGQDRHMIRMRQ | 1.395 | 1927 |
| | R119T, T121E | LIDIVDQLKNYVNDLVPEFLPAPEDVETNCEWSAFSCFQKA | | |
| | | QLKSANTGNNERIINVSIKKLKRKPPSTNAGGGQKHTLECP | | |
| | | SCDSYEKKPPKEFLERFKSLLQKMIHQHLSSRTHGSEDS | | |
| IL-21_5035 | P107V, T110D, | MRSSPGNMERIVICLMVIFLGTLVHKSSSQGQDRHMIRMRQ | 1.397 | 1928 |
| | R115G, T121E | LIDIVDQLKNYVNDLVPEFLPAPEDVETNCEWSAFSCFQKA | | |
| | | QLKSANTGNNERIINVSIKKLKRKVPSDNAGRGQKHRLECP | | |
| | | SCDSYEKKPPKEFLERFKSLLQKMIHQHLSSRTHGSEDS | | |
| IL-21_6299 | H35F, P107V, | MRSSPGNMERIVICLMVIFLGTLVHKSSSQGQDRFMIRMRQ | 1.398 | 1929 |
| | R115D | LIDIVDQLKNYVNDLVPEFLPAPEDVETNCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5395 | N70G, P107V, | MRSSPGNMERIVICLMVIFLGTLVHKSSSQGQDRHMIRMRQ | 1.4 | 1930 |
| | R114Q, H118D | LIDIVDQLKNYVNDLVPEFLPAPEDVETGCEWSAFSCFQKA | | |
| | | QLKSANTGNNERIINVSIKKLKRKVPSTNAGQRQKDRLTCP | | |
| | | SCDSYEKKPPKEFLERFKSLLQKMIHQHLSSRTHGSEDS | | |
| IL-21_5875 | I45T, V98E, | MRSSPGNMERIVICLMVIFLGTLVHKSSSQGQDRHMIRMRQ | 1.401 | 1931 |
| | A112P, R115G | LIDTVDQLKNYVNDLVPEFLPAPEDVETNCEWSAFSCFQKA | | |
| | | QLKSANTGNNERIINESIKKLKRKPPSTNPGRGQKHRLTCP | | |
| | | SCDSYEKKPPKEFLERFKSLLQKMIHQHLSSRTHGSEDS | | |
| IL-21_6065 | H35F, I45T, | MRSSPGNMERIVICLMVIFLGTLVHKSSSQGQDRFMIRMRQ | 1.401 | 1932 |
| | T110D, R114Q | LIDTVDQLKNYVNDLVPEFLPAPEDVETNCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_4927 | A112P, R114G, | MRSSPGNMERIVICLMVIFLGTLVHKSSSQGQDRHMIRMRQ | 1.402 | 1933 |
| | H118D, T121E | LIDIVDQLKNYVNDLVPEFLPAPEDVETNCEWSAFSCFQKA | | |
| | | QLKSANTGNNERIINVSIKKLKRKPPSTNPGGRQKDRLECP | | |
| | | SCDSYEKKPPKEFLERFKSLLQKMIHQHLSSRTHGSEDS | | |
| IL-21_5938 | I45T, N70G, | MRSSPGNMERIVICLMVIFLGTLVHKSSSQGQDRHMIRMRQ | 1.403 | 1934 |
| | V98E, R114Q | LIDTVDQLKNYVNDLVPEFLPAPEDVETGCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_6179 | H35I, N70G, | MRSSPGNMERIVICLMVIFLGTLVHKSSSQGQDRIMIRMRQ | 1.403 | 1935 |
| | P107V, R115G | LIDIVDQLKNYVNDLVPEFLPAPEDVETGCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5918 | I45T, N70G, | MRSSPGNMERIVICLMVIFLGTLVHKSSSQGQDRHMIRMRQ | 1.403 | 1936 |
| | R114Q, R115G | LIDTVDQLKNYVNDLVPEFLPAPEDVETGCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5590 | P107V, R114G, | MRSSPGNMERIVICLMVIFLGTLVHKSSSQGQDRHMIRMRQ | 1.403 | 1937 |
| | H118D | LIDIVDQLKNYVNDLVPEFLPAPEDVETNCEWSAFSCFQKA | | |
| | | QLKSANTGNNERIINVSIKKLKRKVPSTNAGGRQKDRLTCP | | |
| | | SCDSYEKKPPKEFLERFKSLLQKMIHQHLSSRTHGSEDS | | |
| IL-21_4963 | T110D, R114Q, | MRSSPGNMERIVICLMVIFLGTLVHKSSSQGQDRHMIRMRQ | 1.403 | 1938 |
| | R115G, T121E | LIDIVDQLKNYVNDLVPEFLPAPEDVETNCEWSAFSCFQKA | | |
| | | QLKSANTGNNERIINVSIKKLKRKPPSDNAGQGQKHRLECP | | |
| | | SCDSYEKKPPKEFLERFKSLLQKMIHQHLSSRTHGSEDS | | |
| IL-21_5526 | R114Q, H118D, | MRSSPGNMERIVICLMVIFLGTLVHKSSSQGQDRHMIRMRQ | 1.406 | 1939 |
| | T121E | LIDIVDQLKNYVNDLVPEFLPAPEDVETNCEWSAFSCFQKA | | |
| | | QLKSANTGNNERIINVSIKKLKRKPPSTNAGQRQKDRLECP | | |
| | | SCDSYEKKPPKEFLERFKSLLQKMIHQHLSSRTHGSEDS | | |
| IL-21_5570 | T110D, R114Q, | MRSSPGNMERIVICLMVIFLGTLVHKSSSQGQDRHMIRMRQ | 1.407 | 1940 |
| | R115G | LIDIVDQLKNYVNDLVPEFLPAPEDVETNCEWSAFSCFQKA | | |
| | | QLKSANTGNNERIINVSIKKLKRKPPSDNAGQGQKHRLTCP | | |
| | | SCDSYEKKPPKEFLERFKSLLQKMIHQHLSSRTHGSEDS | | |
| IL-21_4974 | T110D, A112P, | MRSSPGNMERIVICLMVIFLGTLVHKSSSQGQDRHMIRMRQ | 1.408 | 1941 |
| | R115G, H118D | LIDIVDQLKNYVNDLVPEFLPAPEDVETNCEWSAFSCFQKA | | |
| | | QLKSANTGNNERIINVSIKKLKRKPPSDNPGRGQKDRLTCP | | |
| | | SCDSYEKKPPKEFLERFKSLLQKMIHQHLSSRTHGSEDS | | |
| IL-21_5863 | I45T, P107V, | MRSSPGNMERIVICLMVIFLGTLVHKSSSQGQDRHMIRMRQ | 1.409 | 1942 |
| | A112P, R115D | LIDTVDQLKNYVNDLVPEFLPAPEDVETNCEWSAFSCFQKA | | |
| | | QLKSANTGNNERIINVSIKKLKRKVPSTNPGRDQKHRLTCP | | |
| | | SCDSYEKKPPKEFLERFKSLLQKMIHQHLSSRTHGSEDS | | |
| IL-21_6028 | H35F, N70G, | MRSSPGNMERIVICLMVIFLGTLVHKSSSQGQDRFMIRMRQ | 1.41 | 1943 |
| | T110D, R115G | LIDIVDQLKNYVNDLVPEFLPAPEDVETGCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_4932 | A112P, R114G, | MRSSPGNMERIVICLMVIFLGTLVHKSSSQGQDRHMIRMRQ | 1.41 | 1944 |
| | R115D, T121E | LIDIVDQLKNYVNDLVPEFLPAPEDVETNCEWSAFSCFQKA | | |
| | | QLKSANTGNNERIINVSIKKLKRKPPSTNPGGDQKHRLECP | | |
| | | SCDSYEKKPPKEFLERFKSLLQKMIHQHLSSRTHGSEDS | | |
| IL-21_5868 | I45T, P107V, | MRSSPGNMERIVICLMVIFLGTLVHKSSSQGQDRHMIRMRQ | 1.411 | 1945 |
| | T110D, R114G | LIDTVDQLKNYVNDLVPEFLPAPEDVETNCEWSAFSCFQKA | | |
| | | QLKSANTGNNERIINVSIKKLKRKVPSDNAGGRQKHRLTCP | | |
| | | SCDSYEKKPPKEFLERFKSLLQKMIHQHLSSRTHGSEDS | | |
| IL-21_5580 | P107V, H118D, | MRSSPGNMERIVICLMVIFLGTLVHKSSSQGQDRHMIRMRQ | 1.411 | 1946 |
| | T121E | LIDIVDQLKNYVNDLVPEFLPAPEDVETNCEWSAFSCFQKA | | |
| | | QLKSANTGNNERIINVSIKKLKRKVPSTNAGRRQKDRLECP | | |
| | | SCDSYEKKPPKEFLERFKSLLQKMIHQHLSSRTHGSEDS | | |
| IL-21_6139 | H35I, V98E, | MRSSPGNMERIVICLMVIFLGTLVHKSSSQGQDRIMIRMRQ | 1.412 | 1947 |
| | P107V, A112P | LIDIVDQLKNYVNDLVPEFLPAPEDVETNCEWSAFSCFQKA | | |
| | | QLKSANTGNNERIINESIKKLKRKVPSTNPGRRQKHRLTCP | | |
| | | SCDSYEKKPPKEFLERFKSLLQKMIHQHLSSRTHGSEDS | | |
| IL-21_6290 | H35F, A112P, | MRSSPGNMERIVICLMVIFLGTLVHKSSSQGQDRFMIRMRQ | 1.416 | 1948 |
| | R115D | LIDIVDQLKNYVNDLVPEFLPAPEDVETNCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5053 | P107V, T110D, | MRSSPGNMERIVICLMVIFLGTLVHKSSSQGQDRHMIRMRQ | 1.417 | 1949 |
| | A112P, H118D | LIDIVDQLKNYVNDLVPEFLPAPEDVETNCEWSAFSCFQKA | | |
| | | QLKSANTGNNERIINVSIKKLKRKVPSDNPGRRQKDRLTCP | | |
| | | SCDSYEKKPPKEFLERFKSLLQKMIHQHLSSRTHGSEDS | | |
| IL-21_6173 | H35I, N70G, | MRSSPGNMERIVICLMVIFLGTLVHKSSSQGQDRIMIRMRQ | 1.417 | 1950 |
| | A112P, R114G | LIDIVDQLKNYVNDLVPEFLPAPEDVETGCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5601 | P107V, A112P, | MRSSPGNMERIVICLMVIFLGTLVHKSSSQGQDRHMIRMRQ | 1.417 | 1951 |
| | R115D | LIDIVDQLKNYVNDLVPEFLPAPEDVETNCEWSAFSCFQKA | | |
| | | QLKSANTGNNERIINVSIKKLKRKVPSTNPGRDQKHRLTCP | | |
| | | SCDSYEKKPPKEFLERFKSLLQKMIHQHLSSRTHGSEDS | | |
| IL-21_5041 | P107V, T110D, | MRSSPGNMERIVICLMVIFLGTLVHKSSSQGQDRHMIRMRQ | 1.42 | 1952 |
| | R114G, T121E | LIDIVDQLKNYVNDLVPEFLPAPEDVETNCEWSAFSCFQKA | | |
| | | QLKSANTGNNERIINVSIKKLKRKVPSDNAGGRQKHRLECP | | |
| | | SCDSYEKKPPKEFLERFKSLLQKMIHQHLSSRTHGSEDS | | |
| IL-21_5340 | N70G, A112P, | MRSSPGNMERIVICLMVIFLGTLVHKSSSQGQDRHMIRMRQ | 1.421 | 1953 |
| | R115D, T121E | LIDIVDQLKNYVNDLVPEFLPAPEDVETGCEWSAFSCFQKA | | |
| | | QLKSANTGNNERIINVSIKKLKRKPPSTNPGRDQKHRLECP | | |
| | | SCDSYEKKPPKEFLERFKSLLQKMIHQHLSSRTHGSEDS | | |
| IL-21_5852 | I45T, T110D, | MRSSPGNMERIVICLMVIFLGTLVHKSSSQGQDRHMIRMRQ | 1.422 | 1954 |
| | R114Q, R115G | LIDTVDQLKNYVNDLVPEFLPAPEDVETNCEWSAFSCFQKA | | |
| | | QLKSANTGNNERIINVSIKKLKRKPPSDNAGQGQKHRLTCP | | |
| | | SCDSYEKKPPKEFLERFKSLLQKMIHQHLSSRTHGSEDS | | |
| IL-21_6389 | H35I, I45T, | MRSSPGNMERIVICLMVIFLGTLVHKSSSQGQDRIMIRMRQ | 1.422 | 1955 |
| | K81D | LIDTVDQLKNYVNDLVPEFLPAPEDVETNCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6033 | H35F, N70G, | MRSSPGNMERIVICLMVIFLGTLVHKSSSQGQDREMIRMRQ | 1.422 | 1956 |
| | P107V, R115G | LIDIVDQLKNYVNDLVPEFLPAPEDVETGCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_6264 | I45T, V98E, | MRSSPGNMERIVICLMVIFLGTLVHKSSSQGQDRHMIRMRQ | 1.422 | 1957 |
| | R114G | LIDTVDQLKNYVNDLVPEFLPAPEDVETNCEWSAFSCFQKA | | |
| | | QLKSANTGNNERIINESIKKLKRKPPSTNAGGRQKHRLTCP | | |
| | | SCDSYEKKPPKEFLERFKSLLQKMIHQHLSSRTHGSEDS | | |
| IL-21_5823 | V98E, P107V | MRSSPGNMERIVICLMVIFLGTLVHKSSSQGQDRHMIRMRQ | 1.424 | 1958 |
| | | LIDIVDQLKNYVNDLVPEFLPAPEDVETNCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_4922 | A112P, R115G, | MRSSPGNMERIVICLMVIFLGTLVHKSSSQGQDRHMIRMRQ | 1.424 | 1959 |
| | H118D, R119T | LIDIVDQLKNYVNDLVPEFLPAPEDVETNCEWSAFSCFQKA | | |
| | | QLKSANTGNNERIINVSIKKLKRKPPSTNPGRGQKDTLTCP | | |
| | | SCDSYEKKPPKEFLERFKSLLQKMIHQHLSSRTHGSEDS | | |
| IL-21_5883 | I45T, V98E, | MRSSPGNMERIVICLMVIFLGTLVHKSSSQGQDRHMIRMRQ | 1.426 | 1960 |
| | T110D, A112P | LIDTVDQLKNYVNDLVPEFLPAPEDVETNCEWSAFSCFQKA | | |
| | | QLKSANTGNNERIINESIKKLKRKPPSDNPGRRQKHRLTCP | | |
| | | SCDSYEKKPPKEFLERFKSLLQKMIHQHLSSRTHGSEDS | | |
| IL-21_5547 | A112P, R114G, | MRSSPGNMERIVICLMVIFLGTLVHKSSSQGQDRHMIRMRQ | 1.426 | 1961 |
| | R115D | LIDIVDQLKNYVNDLVPEFLPAPEDVETNCEWSAFSCFQKA | | |
| | | QLKSANTGNNERIINVSIKKLKRKPPSTNPGGDQKHRLTCP | | |
| | | SCDSYEKKPPKEFLERFKSLLQKMIHQHLSSRTHGSEDS | | |
| IL-21_4930 | A112P, R114G, | MRSSPGNMERIVICLMVIFLGTLVHKSSSQGQDRHMIRMRQ | 1.427 | 1962 |
| | R115G, R119T | LIDIVDQLKNYVNDLVPEFLPAPEDVETNCEWSAFSCFQKA | | |
| | | QLKSANTGNNERIINVSIKKLKRKPPSTNPGGGQKHTLTCP | | |
| | | SCDSYEKKPPKEFLERFKSLLQKMIHQHLSSRTHGSEDS | | |
| IL-21_6375 | H35I, N70G, | MRSSPGNMERIVICLMVIFLGTLVHKSSSQGQDRIMIRMRQ | 1.427 | 1963 |
| | R114Q | LIDIVDQLKNYVNDLVPEFLPAPEDVETGCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_4970 | T110D, A112P, | MRSSPGNMERIVICLMVIFLGTLVHKSSSQGQDRHMIRMRQ | 1.427 | 1964 |
| | H118D, T121E | LIDIVDQLKNYVNDLVPEFLPAPEDVETNCEWSAFSCFQKA | | |
| | | QLKSANTGNNERIINVSIKKLKRKPPSDNPGRRQKDRLECP | | |
| | | SCDSYEKKPPKEFLERFKSLLQKMIHQHLSSRTHGSEDS | | |
| IL-21_5531 | R114Q, R115D, | MRSSPGNMERIVICLMVIFLGTLVHKSSSQGQDRHMIRMRQ | 1.428 | 1965 |
| | T121E | LIDIVDQLKNYVNDLVPEFLPAPEDVETNCEWSAFSCFQKA | | |
| | | QLKSANTGNNERIINVSIKKLKRKPPSTNAGQDQKHRLECP | | |
| | | SCDSYEKKPPKEFLERFKSLLQKMIHQHLSSRTHGSEDS | | |
| IL-21_6184 | H35I, N70G, | MRSSPGNMERIVICLMVIFLGTLVHKSSSQGQDRIMIRMRQ | 1.428 | 1966 |
| | 1P07V, T110D | LIDIVDQLKNYVNDLVPEFLPAPEDVETGCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_6181 | H35I, N70G, | MRSSPGNMERIVICLMVIFLGTLVHKSSSQGQDRIMIRMRQ | 1.428 | 1967 |
| | P107V, R114G | LIDIVDQLKNYVNDLVPEFLPAPEDVETGCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_6273 | I45T, K81D, | MRSSPGNMERIVICLMVIFLGTLVHKSSSQGQDRHMIRMRQ | 1.43 | 1968 |
| | A112P | LIDTVDQLKNYVNDLVPEFLPAPEDVETNCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5542 | A112P, R115D, | MRSSPGNMERIVICLMVIFLGTLVHKSSSQGQDRHMIRMRQ | 1.43 | 1969 |
| | H118D | LIDIVDQLKNYVNDLVPEFLPAPEDVETNCEWSAFSCFQKA | | |
| | | QLKSANTGNNERIINVSIKKLKRKPPSTNPGRDQKDRLTCP | | |
| | | SCDSYEKKPPKEFLERFKSLLQKMIHQHLSSRTHGSEDS | | |
| IL-21_5032 | P107V, T110D, | MRSSPGNMERIVICLMVIFLGTLVHKSSSQGQDRHMIRMRQ | 1.43 | 1970 |
| | R119T, T121E | LIDIVDQLKNYVNDLVPEFLPAPEDVETNCEWSAFSCFQKA | | |
| | | QLKSANTGNNERIINVSIKKLKRKVPSDNAGRRQKHTLECP | | |
| | | SCDSYEKKPPKEFLERFKSLLQKMIHQHLSSRTHGSEDS | | |
| IL-21_5958 | H35F, T110D, | MRSSPGNMERIVICLMVIFLGTLVHKSSSQGQDREMIRMRQ | 1.431 | 1971 |
| | A112P, R115G | LIDIVDQLKNYVNDLVPEFLPAPEDVETNCEWSAFSCFQKA | | |
| | | QLKSANTGNNERIINVSIKKLKRKPPSDNPGRGQKHRLTCP | | |
| | | SCDSYEKKPPKEFLERFKSLLQKMIHQHLSSRTHGSEDS | | |
| IL-21_6388 | H35I, I45T, | MRSSPGNMERIVICLMVIFLGTLVHKSSSQGQDRIMIRMRQ | 1.432 | 1972 |
| | V98E | LIDTVDQLKNYVNDLVPEFLPAPEDVETNCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5527 | R114Q, H118D, | MRSSPGNMERIVICLMVIFLGTLVHKSSSQGQDRHMIRMRQ | 1.433 | 1973 |
| | R119T | LIDIVDQLKNYVNDLVPEFLPAPEDVETNCEWSAFSCFQKA | | |
| | | QLKSANTGNNERIINVSIKKLKRKPPSTNAGQRQKDTLTCP | | |
| | | SCDSYEKKPPKEFLERFKSLLQKMIHQHLSSRTHGSEDS | | |
| IL-21_6352 | H35I, P107V, | MRSSPGNMERIVICLMVIFLGTLVHKSSSQGQDRIMIRMRQ | 1.433 | 1974 |
| | R115D | LIDIVDQLKNYVNDLVPEFLPAPEDVETNCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_6286 | H35F, R114G, | MRSSPGNMERIVICLMVIFLGTLVHKSSSQGQDRFMIRMRQ | 1.434 | 1975 |
| | R115D | LIDIVDQLKNYVNDLVPEFLPAPEDVETNCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_6319 | H35F, N70G, | MRSSPGNMERIVICLMVIFLGTLVHKSSSQGQDRFMIRMRQ | 1.435 | 1976 |
| | R115G | LIDIVDQLKNYVNDLVPEFLPAPEDVETGCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_4921 | A112P, R115G, | MRSSPGNMERIVICLMVIFLGTLVHKSSSQGQDRHMIRMRQ | 1.436 | 1977 |
| | H118D, T121E | LIDIVDQLKNYVNDLVPEFLPAPEDVETNCEWSAFSCFQKA | | |
| | | QLKSANTGNNERIINVSIKKLKRKPPSTNPGRGQKDRLECP | | |
| | | SCDSYEKKPPKEFLERFKSLLQKMIHQHLSSRTHGSEDS | | |
| IL-21_5877 | I45T, V98E, | MRSSPGNMERIVICLMVIFLGTLVHKSSSQGQDRHMIRMRQ | 1.436 | 1978 |
| | A112P, R114G | LIDTVDQLKNYVNDLVPEFLPAPEDVETNCEWSAFSCFQKA | | |
| | | QLKSANTGNNERIINESIKKLKRKPPSTNPGGRQKHRLTCP | | |
| | | SCDSYEKKPPKEFLERFKSLLQKMIHQHLSSRTHGSEDS | | |
| IL-21_5565 | T110D, R114G, | MRSSPGNMERIVICLMVIFLGTLVHKSSSQGQDRHMIRMRQ | 1.438 | 1979 |
| | R115G | LIDIVDQLKNYVNDLVPEFLPAPEDVETNCEWSAFSCFQKA | | |
| | | QLKSANTGNNERIINVSIKKLKRKPPSDNAGGGQKHRLTCP | | |
| | | SCDSYEKKPPKEFLERFKSLLQKMIHQHLSSRTHGSEDS | | |
| IL-21_6182 | H35I, N70G, | MRSSPGNMERIVICLMVIFLGTLVHKSSSQGQDRIMIRMRQ | 1.441 | 1980 |
| | P107V, R114Q | LIDIVDQLKNYVNDLVPEFLPAPEDVETGCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_6329 | H35F, I45T, | MRSSPGNMERIVICLMVIFLGTLVHKSSSQGQDREMIRMRQ | 1.441 | 1981 |
| | R115D | LIDTVDQLKNYVNDLVPEFLPAPEDVETNCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_6402 | H35F, R115D | MRSSPGNMERIVICLMVIFLGTLVHKSSSQGQDRFMIRMRQ | 1.442 | 1982 |
| | | LIDIVDQLKNYVNDLVPEFLPAPEDVETNCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_6171 | H35I, N70G, | MRSSPGNMERIVICLMVIFLGTLVHKSSSQGQDRIMIRMRQ | 1.443 | 1983 |
| | A112P, R115G | LIDIVDQLKNYVNDLVPEFLPAPEDVETGCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5346 | N70G, A112P, | MRSSPGNMERIVICLMVIFLGTLVHKSSSQGQDRHMIRMRQ | 1.444 | 1984 |
| | R114G, R115G | LIDIVDQLKNYVNDLVPEFLPAPEDVETGCEWSAFSCFQKA | | |
| | | QLKSANTGNNERIINVSIKKLKRKPPSTNPGGGQKHRLTCP | | |
| | | SCDSYEKKPPKEFLERFKSLLQKMIHQHLSSRTHGSEDS | | |
| IL-21_6252 | I45T, T110D, | MRSSPGNMERIVICLMVIFLGTLVHKSSSQGQDRHMIRMRQ | 1.446 | 1985 |
| | R115D | LIDTVDQLKNYVNDLVPEFLPAPEDVETNCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6090 | H35F, I45T, | MRSSPGNMERIVICLMVIFLGTLVHKSSSQGQDRFMIRMRQ | 1.448 | 1986 |
| | N70G, R114G | LIDTVDQLKNYVNDLVPEFLPAPEDVETGCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_4973 | T110D, A112P, | MRSSPGNMERIVICLMVIFLGTLVHKSSSQGQDRHMIRMRQ | 1.448 | 1987 |
| | R115G, R119T | LIDIVDQLKNYVNDLVPEFLPAPEDVETNCEWSAFSCFQKA | | |
| | | QLKSANTGNNERIINVSIKKLKRKPPSDNPGRGQKHTLTCP | | |
| | | SCDSYEKKPPKEFLERFKSLLQKMIHQHLSSRTHGSEDS | | |
| IL-21_5713 | N70G, R115G, | MRSSPGNMERIVICLMVIFLGTLVHKSSSQGQDRHMIRMRQ | 1.449 | 1988 |
| | H118D | LIDIVDQLKNYVNDLVPEFLPAPEDVETGCEWSAFSCFQKA | | |
| | | QLKSANTGNNERIINVSIKKLKRKPPSTNAGRGQKDRLTCP | | |
| | | SCDSYEKKPPKEFLERFKSLLQKMIHQHLSSRTHGSEDS | | |
| IL-21_5341 | N70G, A112P, | MRSSPGNMERIVICLMVIFLGTLVHKSSSQGQDRHMIRMRQ | 1.449 | 1989 |
| | R115D, R119T | LIDIVDQLKNYVNDLVPEFLPAPEDVETGCEWSAFSCFQKA | | |
| | | QLKSANTGNNERIINVSIKKLKRKPPSTNPGRDQKHTLTCP | | |
| | | SCDSYEKKPPKEFLERFKSLLQKMIHQHLSSRTHGSEDS | | |
| IL-21_5710 | N70G, H118D, | MRSSPGNMERIVICLMVIFLGTLVHKSSSQGQDRHMIRMRQ | 1.451 | 1990 |
| | R119T | LIDIVDQLKNYVNDLVPEFLPAPEDVETGCEWSAFSCFQKA | | |
| | | QLKSANTGNNERIINVSIKKLKRKPPSTNAGRRQKDTLTCP | | |
| | | SCDSYEKKPPKEFLERFKSLLQKMIHQHLSSRTHGSEDS | | |
| IL-21_5576 | T110D, A112P, | MRSSPGNMERIVICLMVIFLGTLVHKSSSQGQDRHMIRMRQ | 1.452 | 1991 |
| | R115D | LIDIVDQLKNYVNDLVPEFLPAPEDVETNCEWSAFSCFQKA | | |
| | | QLKSANTGNNERIINVSIKKLKRKPPSDNPGRDQKHRLTCP | | |
| | | SCDSYEKKPPKEFLERFKSLLQKMIHQHLSSRTHGSEDS | | |
| IL-21_5858 | I45T, P107V, | MRSSPGNMERIVICLMVIFLGTLVHKSSSQGQDRHMIRMRQ | 1.452 | 1992 |
| | R114G, R115G | LIDTVDQLKNYVNDLVPEFLPAPEDVETNCEWSAFSCFQKA | | |
| | | QLKSANTGNNERIINVSIKKLKRKVPSTNAGGGQKHRLTCP | | |
| | | SCDSYEKKPPKEFLERFKSLLQKMIHQHLSSRTHGSEDS | | |
| IL-21_6027 | H35F, N70G, | MRSSPGNMERIVICLMVIFLGTLVHKSSSQGQDREMIRMRQ | 1.456 | 1993 |
| | A112P, R114Q | LIDIVDQLKNYVNDLVPEFLPAPEDVETGCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5555 | T110D, H118D, | MRSSPGNMERIVICLMVIFLGTLVHKSSSQGQDRHMIRMRQ | 1.458 | 1994 |
| | R119T | LIDIVDQLKNYVNDLVPEFLPAPEDVETNCEWSAFSCFQKA | | |
| | | QLKSANTGNNERIINVSIKKLKRKPPSDNAGRRQKDTLTCP | | |
| | | SCDSYEKKPPKEFLERFKSLLQKMIHQHLSSRTHGSEDS | | |
| IL-21_5552 | A112P, R114Q, | MRSSPGNMERIVICLMVIFLGTLVHKSSSQGQDRHMIRMRQ | 1.461 | 1995 |
| | R115D | LIDIVDQLKNYVNDLVPEFLPAPEDVETNCEWSAFSCFQKA | | |
| | | QLKSANTGNNERIINVSIKKLKRKPPSTNPGQDQKHRLTCP | | |
| | | SCDSYEKKPPKEFLERFKSLLQKMIHQHLSSRTHGSEDS | | |
| IL-21_6338 | H35I, R114G, | MRSSPGNMERIVICLMVIFLGTLVHKSSSQGQDRIMIRMRQ | 1.461 | 1996 |
| | R115G | LIDIVDQLKNYVNDLVPEFLPAPEDVETNCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5779 | R115D, H118D | MRSSPGNMERIVICLMVIFLGTLVHKSSSQGQDRHMIRMRQ | 1.461 | 1997 |
| | | LIDIVDQLKNYVNDLVPEFLPAPEDVETNCEWSAFSCFQKA | | |
| | | QLKSANTGNNERIINVSIKKLKRKPPSTNAGRDQKDRLTCP | | |
| | | SCDSYEKKPPKEFLERFKSLLQKMIHQHLSSRTHGSEDS | | |
| IL-21_5353 | N70G, T110D, | MRSSPGNMERIVICLMVIFLGTLVHKSSSQGQDRHMIRMRQ | 1.462 | 1998 |
| | R119T, T121E | LIDIVDQLKNYVNDLVPEFLPAPEDVETGCEWSAFSCFQKA | | |
| | | QLKSANTGNNERIINVSIKKLKRKPPSDNAGRRQKHTLECP | | |
| | | SCDSYEKKPPKEFLERFKSLLQKMIHQHLSSRTHGSEDS | | |
| IL-21_5736 | N70G, T110D, | MRSSPGNMERIVICLMVIFLGTLVHKSSSQGQDRHMIRMRQ | 1.463 | 1999 |
| | H118D | LIDIVDQLKNYVNDLVPEFLPAPEDVETGCEWSAFSCFQKA | | |
| | | QLKSANTGNNERIINVSIKKLKRKPPSDNAGRRQKDRLTCP | | |
| | | SCDSYEKKPPKEFLERFKSLLQKMIHQHLSSRTHGSEDS | | |
| IL-21_5558 | T110D, R115G, | MRSSPGNMERIVICLMVIFLGTLVHKSSSQGQDRHMIRMRQ | 1.464 | 2000 |
| | H118D | LIDIVDQLKNYVNDLVPEFLPAPEDVETNCEWSAFSCFQKA | | |
| | | QLKSANTGNNERIINVSIKKLKRKPPSDNAGRGQKDRLTCP | | |
| | | SCDSYEKKPPKEFLERFKSLLQKMIHQHLSSRTHGSEDS | | |
| IL-21_5709 | N70G, H118D, | MRSSPGNMERIVICLMVIFLGTLVHKSSSQGQDRHMIRMRQ | 1.465 | 2001 |
| | T121E | LIDIVDQLKNYVNDLVPEFLPAPEDVETGCEWSAFSCFQKA | | |
| | | QLKSANTGNNERIINVSIKKLKRKPPSTNAGRRQKDRLECP | | |
| | | SCDSYEKKPPKEFLERFKSLLQKMIHQHLSSRTHGSEDS | | |
| IL-21_6174 | H35I, N70G, | MRSSPGNMERIVICLMVIFLGTLVHKSSSQGQDRIMIRMRQ | 1.468 | 2002 |
| | A112P, R114Q | LIDIVDQLKNYVNDLVPEFLPAPEDVETGCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_4986 | T110D, A112P, | MRSSPGNMERIVICLMVIFLGTLVHKSSSQGQDRHMIRMRQ | 1.469 | 2003 |
| | R114Q, R115G | LIDIVDQLKNYVNDLVPEFLPAPEDVETNCEWSAFSCFQKA | | |
| | | QLKSANTGNNERIINVSIKKLKRKPPSDNPGQGQKHRLTCP | | |
| | | SCDSYEKKPPKEFLERFKSLLQKMIHQHLSSRTHGSEDS | | |
| IL-21_5960 | H35F, T110D, | MRSSPGNMERIVICLMVIFLGTLVHKSSSQGQDREMIRMRQ | 1.471 | 2004 |
| | A112P, R114G | LIDIVDQLKNYVNDLVPEFLPAPEDVETNCEWSAFSCFQKA | | |
| | | QLKSANTGNNERIINVSIKKLKRKPPSDNPGGRQKHRLTCP | | |
| | | SCDSYEKKPPKEFLERFKSLLQKMIHQHLSSRTHGSEDS | | |
| IL-21_6105 | H35I, T110D, | MRSSPGNMERIVICLMVIFLGTLVHKSSSQGQDRIMIRMRQ | 1.471 | 2005 |
| | A112P, R115G | LIDIVDQLKNYVNDLVPEFLPAPEDVETNCEWSAFSCFQKA | | |
| | | QLKSANTGNNERIINVSIKKLKRKPPSDNPGRGQKHRLTCP | | |
| | | SCDSYEKKPPKEFLERFKSLLQKMIHQHLSSRTHGSEDS | | |
| IL-21_6223 | H35I, I45T, | MRSSPGNMERIVICLMVIFLGTLVHKSSSQGQDRIMIRMRQ | 1.472 | 2006 |
| | V98E, A112P | LIDTVDQLKNYVNDLVPEFLPAPEDVETNCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5606 | P107V, T110D, | MRSSPGNMERIVICLMVIFLGTLVHKSSSQGQDRHMIRMRQ | 1.473 | 2007 |
| | H118D | LIDIVDQLKNYVNDLVPEFLPAPEDVETNCEWSAFSCFQKA | | |
| | | QLKSANTGNNERIINVSIKKLKRKVPSDNAGRRQKDRLTCP | | |
| | | SCDSYEKKPPKEFLERFKSLLQKMIHQHLSSRTHGSEDS | | |
| IL-21_5789 | R114Q, R115D | MRSSPGNMERIVICLMVIFLGTLVHKSSSQGQDRHMIRMRQ | 1.476 | 2008 |
| | | LIDIVDQLKNYVNDLVPEFLPAPEDVETNCEWSAFSCFQKA | | |
| | | QLKSANTGNNERIINVSIKKLKRKPPSTNAGQDQKHRLTCP | | |
| | | SCDSYEKKPPKEFLERFKSLLQKMIHQHLSSRTHGSEDS | | |
| IL-21_6064 | H35F, I45T, | MRSSPGNMERIVICLMVIFLGTLVHKSSSQGQDREMIRMRQ | 1.477 | 2009 |
| | T110D, R114G | LIDTVDQLKNYVNDLVPEFLPAPEDVETNCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5054 | P107V, T110D, | MRSSPGNMERIVICLMVIFLGTLVHKSSSQGQDRHMIRMRQ | 1.477 | 2010 |
| | A112P, R115G | LIDIVDQLKNYVNDLVPEFLPAPEDVETNCEWSAFSCFQKA | | |
| | | QLKSANTGNNERIINVSIKKLKRKVPSDNPGRGQKHRLTCP | | |
| | | SCDSYEKKPPKEFLERFKSLLQKMIHQHLSSRTHGSEDS | | |
| IL-21_5522 | R114G, R115D, | MRSSPGNMERIVICLMVIFLGTLVHKSSSQGQDRHMIRMRQ | 1.477 | 2011 |
| | T121E | LIDIVDQLKNYVNDLVPEFLPAPEDVETNCEWSAFSCFQKA | | |
| | | QLKSANTGNNERIINVSIKKLKRKPPSTNAGGDQKHRLECP | | |
| | | SCDSYEKKPPKEFLERFKSLLQKMIHQHLSSRTHGSEDS | | |
| IL-21_6116 | H35I, P107V, | MRSSPGNMERIVICLMVIFLGTLVHKSSSQGQDRIMIRMRQ | 1.478 | 2012 |
| | A112P, R114Q | LIDIVDQLKNYVNDLVPEFLPAPEDVETNCEWSAFSCFQKA | | |
| | | QLKSANTGNNERIINVSIKKLKRKVPSTNPGQRQKHRLTCP | | |
| | | SCDSYEKKPPKEFLERFKSLLQKMIHQHLSSRTHGSEDS | | |
| IL-21_5541 | A112P, R115D, | MRSSPGNMERIVICLMVIFLGTLVHKSSSQGQDRHMIRMRQ | 1.478 | 2013 |
| | R119T | LIDIVDQLKNYVNDLVPEFLPAPEDVETNCEWSAFSCFQKA | | |
| | | QLKSANTGNNERIINVSIKKLKRKPPSTNPGRDQKHTLTCP | | |
| | | SCDSYEKKPPKEFLERFKSLLQKMIHQHLSSRTHGSEDS | | |
| IL-21_5350 | N70G, A112P, | MRSSPGNMERIVICLMVIFLGTLVHKSSSQGQDRHMIRMRQ | 1.479 | 2014 |
| | R114Q, H118D | LIDIVDQLKNYVNDLVPEFLPAPEDVETGCEWSAFSCFQKA | | |
| | | QLKSANTGNNERIINVSIKKLKRKPPSTNPGQRQKDRLTCP | | |
| | | SCDSYEKKPPKEFLERFKSLLQKMIHQHLSSRTHGSEDS | | |
| IL-21_6265 | I45T, V98E, | MRSSPGNMERIVICLMVIFLGTLVHKSSSQGQDRHMIRMRQ | 1.481 | 2015 |
| | R114Q | LIDTVDQLKNYVNDLVPEFLPAPEDVETNCEWSAFSCFQKA | | |
| | | QLKSANTGNNERIINESIKKLKRKPPSTNAGQRQKHRLTCP | | |
| | | SCDSYEKKPPKEFLERFKSLLQKMIHQHLSSRTHGSEDS | | |
| IL-21_5724 | N70G, R114Q, | MRSSPGNMERIVICLMVIFLGTLVHKSSSQGQDRHMIRMRQ | 1.483 | 2016 |
| | H118D | LIDIVDQLKNYVNDLVPEFLPAPEDVETGCEWSAFSCFQKA | | |
| | | QLKSANTGNNERIINVSIKKLKRKPPSTNAGQRQKDRLTCP | | |
| | | SCDSYEKKPPKEFLERFKSLLQKMIHQHLSSRTHGSEDS | | |
| IL-21_5046 | P107V, T110D, | MRSSPGNMERIVICLMVIFLGTLVHKSSSQGQDRHMIRMRQ | 1.483 | 2017 |
| | R114Q, T121E | LIDIVDQLKNYVNDLVPEFLPAPEDVETNCEWSAFSCFQKA | | |
| | | QLKSANTGNNERIINVSIKKLKRKVPSDNAGQRQKHRLECP | | |
| | | SCDSYEKKPPKEFLERFKSLLQKMIHQHLSSRTHGSEDS | | |
| IL-21_4919 | A112P, H118D, | MRSSPGNMERIVICLMVIFLGTLVHKSSSQGQDRHMIRMRQ | 1.484 | 2018 |
| | R119T, T121E | LIDIVDQLKNYVNDLVPEFLPAPEDVETNCEWSAFSCFQKA | | |
| | | QLKSANTGNNERIINVSIKKLKRKPPSTNPGRRQKDTLECP | | |
| | | SCDSYEKKPPKEFLERFKSLLQKMIHQHLSSRTHGSEDS | | |
| IL-21_5966 | H35F, P107V, | MRSSPGNMERIVICLMVIFLGTLVHKSSSQGQDREMIRMRQ | 1.485 | 2019 |
| | A112P, R115G | LIDIVDQLKNYVNDLVPEFLPAPEDVETNCEWSAFSCFQKA | | |
| | | QLKSANTGNNERIINVSIKKLKRKVPSTNPGRGQKHRLTCP | | |
| | | SCDSYEKKPPKEFLERFKSLLQKMIHQHLSSRTHGSEDS | | |
| IL-21_5401 | N70G, P107V, | MRSSPGNMERIVICLMVIFLGTLVHKSSSQGQDRHMIRMRQ | 1.485 | 2020 |
| | A112P, R115G | LIDIVDQLKNYVNDLVPEFLPAPEDVETGCEWSAFSCFQKA | | |
| | | QLKSANTGNNERIINVSIKKLKRKVPSTNPGRGQKHRLTCP | | |
| | | SCDSYEKKPPKEFLERFKSLLQKMIHQHLSSRTHGSEDS | | |
| IL-21_5784 | R114G, R115D | MRSSPGNMERIVICLMVIFLGTLVHKSSSQGQDRHMIRMRQ | 1.486 | 2021 |
| | | LIDIVDQLKNYVNDLVPEFLPAPEDVETNCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_6208 | H351, I45T, | MRSSPGNMERIVICLMVIFLGTLVHKSSSQGQDRIMIRMRQ | 1.49 | 2022 |
| | T110D, R115G | LIDTVDQLKNYVNDLVPEFLPAPEDVETNCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_5739 | N70G, T110D, | MRSSPGNMERIVICLMVIFLGTLVHKSSSQGQDRHMIRMRQ | 1.492 | 2023 |
| | R114G | LIDIVDQLKNYVNDLVPEFLPAPEDVETGCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5930 | I45T, N70G, | MRSSPGNMERIVICLMVIFLGTLVHKSSSQGQDRHMIRMRQ | 1.492 | 2024 |
| | P107V, R115D | LIDTVDQLKNYVNDLVPEFLPAPEDVETGCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_4971 | T110D, A112P, | MRSSPGNMERIVICLMVIFLGTLVHKSSSQGQDRHMIRMRQ | 1.494 | 2025 |
| | H118D, R119T | LIDIVDQLKNYVNDLVPEFLPAPEDVETNCEWSAFSCFQKA | | |
| | | QLKSANTGNNERIINVSIKKLKRKPPSDNPGRRQKDTLTCP | | |
| | | SCDSYEKKPPKEFLERFKSLLQKMIHQHLSSRTHGSEDS | | |
| IL-21_5607 | P107V, T110D, | MRSSPGNMERIVICLMVIFLGTLVHKSSSQGQDRHMIRMRQ | 1.496 | 2026 |
| | R115G | LIDIVDQLKNYVNDLVPEFLPAPEDVETNCEWSAFSCFQKA | | |
| | | QLKSANTGNNERIINVSIKKLKRKVPSDNAGRGQKHRLTCP | | |
| | | SCDSYEKKPPKEFLERFKSLLQKMIHQHLSSRTHGSEDS | | |
| IL-21_5056 | P107V, T110D, | MRSSPGNMERIVICLMVIFLGTLVHKSSSQGQDRHMIRMRQ | 1.497 | 2027 |
| | A112P, R114G | LIDIVDQLKNYVNDLVPEFLPAPEDVETNCEWSAFSCFQKA | | |
| | | QLKSANTGNNERIINVSIKKLKRKVPSDNPGGRQKHRLTCP | | |
| | | SCDSYEKKPPKEFLERFKSLLQKMIHQHLSSRTHGSEDS | | |
| IL-21_4980 | T110D, A112P, | MRSSPGNMERIVICLMVIFLGTLVHKSSSQGQDRHMIRMRQ | 1.498 | 2028 |
| | R114G, H118D | LIDIVDQLKNYVNDLVPEFLPAPEDVETNCEWSAFSCFQKA | | |
| | | QLKSANTGNNERIINVSIKKLKRKPPSDNPGGRQKDRLTCP | | |
| | | SCDSYEKKPPKEFLERFKSLLQKMIHQHLSSRTHGSEDS | | |
| IL-21_5916 | I45T, N70G, | MRSSPGNMERIVICLMVIFLGTLVHKSSSQGQDRHMIRMRQ | 1.499 | 2029 |
| | R114G, R115G | LIDTVDQLKNYVNDLVPEFLPAPEDVETGCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5511 | R115G, H118D, | MRSSPGNMERIVICLMVIFLGTLVHKSSSQGQDRHMIRMRQ | 1.504 | 2030 |
| | T121E | LIDIVDQLKNYVNDLVPEFLPAPEDVETNCEWSAFSCFQKA | | |
| | | QLKSANTGNNERIINVSIKKLKRKPPSTNAGRGQKDRLECP | | |
| | | SCDSYEKKPPKEFLERFKSLLQKMIHQHLSSRTHGSEDS | | |
| IL-21_5924 | I45T, N70G, | MRSSPGNMERIVICLMVIFLGTLVHKSSSQGQDRHMIRMRQ | 1.504 | 2031 |
| | T110D, R115G | LIDTVDQLKNYVNDLVPEFLPAPEDVETGCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_6257 | I45T, P107V, | MRSSPGNMERIVICLMVIFLGTLVHKSSSQGQDRHMIRMRQ | 1.504 | 2032 |
| | R115D | LIDTVDQLKNYVNDLVPEFLPAPEDVETNCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_6343 | H35I, A112P, | MRSSPGNMERIVICLMVIFLGTLVHKSSSQGQDRIMIRMRQ | 1.506 | 2033 |
| | R115D | LIDIVDQLKNYVNDLVPEFLPAPEDVETNCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_4937 | A112P, R114Q, | MRSSPGNMERIVICLMVIFLGTLVHKSSSQGQDRHMIRMRQ | 1.506 | 2034 |
| | H118D, R119T | LIDIVDQLKNYVNDLVPEFLPAPEDVETNCEWSAFSCFQKA | | |
| | | QLKSANTGNNERIINVSIKKLKRKPPSTNPGQRQKDTLTCP | | |
| | | SCDSYEKKPPKEFLERFKSLLQKMIHQHLSSRTHGSEDS | | |
| IL-21_5760 | N70G, V98E, | MRSSPGNMERIVICLMVIFLGTLVHKSSSQGQDRHMIRMRQ | 1.508 | 2035 |
| | P107V | LIDIVDQLKNYVNDLVPEFLPAPEDVETGCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5748 | N70G, P107V, | MRSSPGNMERIVICLMVIFLGTLVHKSSSQGQDRHMIRMRQ | 1.509 | 2036 |
| | R114Q | LIDIVDQLKNYVNDLVPEFLPAPEDVETGCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5588 | P107V, R114G, | MRSSPGNMERIVICLMVIFLGTLVHKSSSQGQDRHMIRMRQ | 1.509 | 2037 |
| | T121E | LIDIVDQLKNYVNDLVPEFLPAPEDVETNCEWSAFSCFQKA | | |
| | | QLKSANTGNNERIINVSIKKLKRKVPSTNAGGRQKHRLECP | | |
| | | SCDSYEKKPPKEFLERFKSLLQKMIHQHLSSRTHGSEDS | | |
| IL-21_5024 | P107V, A112P, | MRSSPGNMERIVICLMVIFLGTLVHKSSSQGQDRHMIRMRQ | 1.51 | 2038 |
| | R114G, H118D | LIDIVDQLKNYVNDLVPEFLPAPEDVETNCEWSAFSCFQKA | | |
| | | QLKSANTGNNERIINVSIKKLKRKVPSTNPGGRQKDRLTCP | | |
| | | SCDSYEKKPPKEFLERFKSLLQKMIHQHLSSRTHGSEDS | | |
| IL-21_5740 | N70G, T110D, | MRSSPGNMERIVICLMVIFLGTLVHKSSSQGQDRHMIRMRQ | 1.512 | 2039 |
| | R114Q | LIDIVDQLKNYVNDLVPEFLPAPEDVETGCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_6038 | H35F, N70G, | MRSSPGNMERIVICLMVIFLGTLVHKSSSQGQDREMIRMRQ | 1.512 | 2040 |
| | P107V, T110D | LIDIVDQLKNYVNDLVPEFLPAPEDVETGCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_6298 | H35F, P107V, | MRSSPGNMERIVICLMVIFLGTLVHKSSSQGQDRFMIRMRQ | 1.513 | 2041 |
| | R115G | LIDIVDQLKNYVNDLVPEFLPAPEDVETNCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5814 | V98E, T121E | MRSSPGNMERIVICLMVIFLGTLVHKSSSQGQDRHMIRMRQ | 1.513 | 2042 |
| | | LIDIVDQLKNYVNDLVPEFLPAPEDVETNCEWSAFSCFQKA | | |
| | | QLKSANTGNNERIINESIKKLKRKPPSTNAGRRQKHRLECP | | |
| | | SCDSYEKKPPKEFLERFKSLLQKMIHQHLSSRTHGSEDS | | |
| IL-21_6335 | H35F, I45T, | MRSSPGNMERIVICLMVIFLGTLVHKSSSQGQDRFMIRMRQ | 1.514 | 2043 |
| | V98E | LIDTVDQLKNYVNDLVPEFLPAPEDVETNCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_6211 | H351, I45T, | MRSSPGNMERIVICLMVIFLGTLVHKSSSQGQDRIMIRMRQ | 1.516 | 2044 |
| | T110D, R114Q | LIDTVDQLKNYVNDLVPEFLPAPEDVETNCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5517 | R114G, H118D, | MRSSPGNMERIVICLMVIFLGTLVHKSSSQGQDRHMIRMRQ | 1.517 | 2045 |
| | T121E | LIDIVDQLKNYVNDLVPEFLPAPEDVETNCEWSAFSCFQKA | | |
| | | QLKSANTGNNERIINVSIKKLKRKPPSTNAGGRQKDRLECP | | |
| | | SCDSYEKKPPKEFLERFKSLLQKMIHQHLSSRTHGSEDS | | |
| IL-21_5847 | I45T, A112P, | MRSSPGNMERIVICLMVIFLGTLVHKSSSQGQDRHMIRMRQ | 1.517 | 2046 |
| | R114G, R115D | LIDTVDQLKNYVNDLVPEFLPAPEDVETNCEWSAFSCFQKA | | |
| | | QLKSANTGNNERIINVSIKKLKRKPPSTNPGGDQKHRLTCP | | |
| | | SCDSYEKKPPKEFLERFKSLLQKMIHQHLSSRTHGSEDS | | |
| IL-21_5337 | N70G, A112P, | MRSSPGNMERIVICLMVIFLGTLVHKSSSQGQDRHMIRMRQ | 1.519 | 2047 |
| | R115G, T121E | LIDIVDQLKNYVNDLVPEFLPAPEDVETGCEWSAFSCFQKA | | |
| | | QLKSANTGNNERIINVSIKKLKRKPPSTNPGRGQKHRLECP | | |
| | | SCDSYEKKPPKEFLERFKSLLQKMIHQHLSSRTHGSEDS | | |
| IL-21_6322 | H35F, N70G, | MRSSPGNMERIVICLMVIFLGTLVHKSSSQGQDRFMIRMRQ | 1.52 | 2048 |
| | R114Q | LIDIVDQLKNYVNDLVPEFLPAPEDVETGCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_4975 | T110D, A112P, | MRSSPGNMERIVICLMVIFLGTLVHKSSSQGQDRHMIRMRQ | 1.52 | 2049 |
| | R115D, T121E | LIDIVDQLKNYVNDLVPEFLPAPEDVETNCEWSAFSCFQKA | | |
| | | QLKSANTGNNERIINVSIKKLKRKPPSDNPGRDQKHRLECP | | |
| | | SCDSYEKKPPKEFLERFKSLLQKMIHQHLSSRTHGSEDS | | |
| IL-21_5927 | I45T, N70G, | MRSSPGNMERIVICLMVIFLGTLVHKSSSQGQDRHMIRMRQ | 1.522 | 2050 |
| | T110D, R114Q | LIDTVDQLKNYVNDLVPEFLPAPEDVETGCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5850 | I45T, T110D, | MRSSPGNMERIVICLMVIFLGTLVHKSSSQGQDRHMIRMRQ | 1.523 | 2051 |
| | R114G, R115G | LIDTVDQLKNYVNDLVPEFLPAPEDVETNCEWSAFSCFQKA | | |
| | | QLKSANTGNNERIINVSIKKLKRKPPSDNAGGGQKHRLTCP | | |
| | | SCDSYEKKPPKEFLERFKSLLQKMIHQHLSSRTHGSEDS | | |
| IL-21_5521 | R114G, R115G, | MRSSPGNMERIVICLMVIFLGTLVHKSSSQGQDRHMIRMRQ | 1.524 | 2052 |
| | H118D | LIDIVDQLKNYVNDLVPEFLPAPEDVETNCEWSAFSCFQKA | | |
| | | QLKSANTGNNERIINVSIKKLKRKPPSTNAGGGQKDRLTCP | | |
| | | SCDSYEKKPPKEFLERFKSLLQKMIHQHLSSRTHGSEDS | | |
| IL-21_6036 | H35F, N70G, | MRSSPGNMERIVICLMVIFLGTLVHKSSSQGQDREMIRMRQ | 1.527 | 2053 |
| | P107V, R114Q | LIDIVDQLKNYVNDLVPEFLPAPEDVETGCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_4960 | T110D, R114Q, | MRSSPGNMERIVICLMVIFLGTLVHKSSSQGQDRHMIRMRQ | 1.528 | 2054 |
| | R119T, T121E | LIDIVDQLKNYVNDLVPEFLPAPEDVETNCEWSAFSCFQKA | | |
| | | QLKSANTGNNERIINVSIKKLKRKPPSDNAGQRQKHTLECP | | |
| | | SCDSYEKKPPKEFLERFKSLLQKMIHQHLSSRTHGSEDS | | |
| IL-21_6321 | H35F, N70G, | MRSSPGNMERIVICLMVIFLGTLVHKSSSQGQDRFMIRMRQ | 1.529 | 2055 |
| | R114G | LIDIVDQLKNYVNDLVPEFLPAPEDVETGCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5878 | I45T, V98E, | MRSSPGNMERIVICLMVIFLGTLVHKSSSQGQDRHMIRMRQ | 1.53 | 2056 |
| | A112P, R114Q | LIDTVDQLKNYVNDLVPEFLPAPEDVETNCEWSAFSCFQKA | | |
| | | QLKSANTGNNERIINESIKKLKRKPPSTNPGQRQKHRLTCP | | |
| | | SCDSYEKKPPKEFLERFKSLLQKMIHQHLSSRTHGSEDS | | |
| IL-21_6382 | H35I, I45T, | MRSSPGNMERIVICLMVIFLGTLVHKSSSQGQDRIMIRMRQ | 1.53 | 2057 |
| | R115D | LIDTVDQLKNYVNDLVPEFLPAPEDVETNCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5336 | N70G, A112P, | MRSSPGNMERIVICLMVIFLGTLVHKSSSQGQDRHMIRMRQ | 1.532 | 2058 |
| | H118D, R119T | LIDIVDQLKNYVNDLVPEFLPAPEDVETGCEWSAFSCFQKA | | |
| | | QLKSANTGNNERIINVSIKKLKRKPPSTNPGRRQKDTLTCP | | |
| | | SCDSYEKKPPKEFLERFKSLLQKMIHQHLSSRTHGSEDS | | |
| IL-21_6035 | H35F, N70G, | MRSSPGNMERIVICLMVIFLGTLVHKSSSQGQDRFMIRMRQ | 1.533 | 2059 |
| | P107V, R114G | LIDIVDQLKNYVNDLVPEFLPAPEDVETGCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_6216 | H35I, I45T, | MRSSPGNMERIVICLMVIFLGTLVHKSSSQGQDRIMIRMRQ | 1.533 | 2060 |
| | P107V, R114Q | LIDTVDQLKNYVNDLVPEFLPAPEDVETNCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_4984 | T110D, A112P, | MRSSPGNMERIVICLMVIFLGTLVHKSSSQGQDRHMIRMRQ | 1.535 | 2061 |
| | R114Q, R119T | LIDIVDQLKNYVNDLVPEFLPAPEDVETNCEWSAFSCFQKA | | |
| | | QLKSANTGNNERIINVSIKKLKRKPPSDNPGQRQKHTLTCP | | |
| | | SCDSYEKKPPKEFLERFKSLLQKMIHQHLSSRTHGSEDS | | |
| IL-21_5344 | N70G, A112P, | MRSSPGNMERIVICLMVIFLGTLVHKSSSQGQDRHMIRMRQ | 1.537 | 2062 |
| | R114G, R119T | LIDIVDQLKNYVNDLVPEFLPAPEDVETGCEWSAFSCFQKA | | |
| | | QLKSANTGNNERIINVSIKKLKRKPPSTNPGGRQKHTLTCP | | |
| | | SCDSYEKKPPKEFLERFKSLLQKMIHQHLSSRTHGSEDS | | |
| IL-21_6225 | H35I, I45T, | MRSSPGNMERIVICLMVIFLGTLVHKSSSQGQDRIMIRMRQ | 1.538 | 2063 |
| | V98E, P107V | LIDTVDQLKNYVNDLVPEFLPAPEDVETNCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5921 | I45T, N70G, | MRSSPGNMERIVICLMVIFLGTLVHKSSSQGQDRHMIRMRQ | 1.54 | 2064 |
| | A112P, R115D | LIDTVDQLKNYVNDLVPEFLPAPEDVETGCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5821 | V98E, A112P | MRSSPGNMERIVICLMVIFLGTLVHKSSSQGQDRHMIRMRQ | 1.543 | 2065 |
| | | LIDIVDQLKNYVNDLVPEFLPAPEDVETNCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5809 | P107V, R115D | MRSSPGNMERIVICLMVIFLGTLVHKSSSQGQDRHMIRMRQ | 1.545 | 2066 |
| | | LIDIVDQLKNYVNDLVPEFLPAPEDVETNCEWSAFSCFQKA | | |
| | | QLKSANTGNNERIINVSIKKLKRKVPSTNAGRDQKHRLTCP | | |
| | | SCDSYEKKPPKEFLERFKSLLQKMIHQHLSSRTHGSEDS | | |
| IL-21_5941 | I45T, N70G, | MRSSPGNMERIVICLMVIFLGTLVHKSSSQGQDRHMIRMRQ | 1.546 | 2067 |
| | V98E, P107V | LIDTVDQLKNYVNDLVPEFLPAPEDVETGCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5382 | N70G, P107V, | MRSSPGNMERIVICLMVIFLGTLVHKSSSQGQDRHMIRMRQ | 1.546 | 2068 |
| | R115G, T121E | LIDIVDQLKNYVNDLVPEFLPAPEDVETGCEWSAFSCFQKA | | |
| | | QLKSANTGNNERIINVSIKKLKRKVPSTNAGRGQKHRLECP | | |
| | | SCDSYEKKPPKEFLERFKSLLQKMIHQHLSSRTHGSEDS | | |
| IL-21_4979 | T110D, A112P, | MRSSPGNMERIVICLMVIFLGTLVHKSSSQGQDRHMIRMRQ | 1.548 | 2069 |
| | R114G, R119T | LIDIVDQLKNYVNDLVPEFLPAPEDVETNCEWSAFSCFQKA | | |
| | | QLKSANTGNNERIINVSIKKLKRKPPSDNPGGRQKHTLTCP | | |
| | | SCDSYEKKPPKEFLERFKSLLQKMIHQHLSSRTHGSEDS | | |
| IL-21_6403 | H35F, R114G | MRSSPGNMERIVICLMVIFLGTLVHKSSSQGQDREMIRMRQ | 1.548 | 2070 |
| | | LIDIVDQLKNYVNDLVPEFLPAPEDVETNCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5509 | H118D, R119T, | MRSSPGNMERIVICLMVIFLGTLVHKSSSQGQDRHMIRMRQ | 1.55 | 2071 |
| | T121E | LIDIVDQLKNYVNDLVPEFLPAPEDVETNCEWSAFSCFQKA | | |
| | | QLKSANTGNNERIINVSIKKLKRKPPSTNAGRRQKDTLECP | | |
| | | SCDSYEKKPPKEFLERFKSLLQKMIHQHLSSRTHGSEDS | | |
| IL-21_4981 | T110D, A112P, | MRSSPGNMERIVICLMVIFLGTLVHKSSSQGQDRHMIRMRQ | 1.55 | 2072 |
| | R114G, R115G | LIDIVDQLKNYVNDLVPEFLPAPEDVETNCEWSAFSCFQKA | | |
| | | QLKSANTGNNERIINVSIKKLKRKPPSDNPGGGQKHRLTCP | | |
| | | SCDSYEKKPPKEFLERFKSLLQKMIHQHLSSRTHGSEDS | | |
| IL-21_6401 | H35F, R115G | MRSSPGNMERIVICLMVIFLGTLVHKSSSQGQDRFMIRMRQ | 1.552 | 2073 |
| | | LIDIVDQLKNYVNDLVPEFLPAPEDVETNCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5723 | N70G, R114Q, | MRSSPGNMERIVICLMVIFLGTLVHKSSSQGQDRHMIRMRQ | 1.554 | 2074 |
| | R119T | LIDIVDQLKNYVNDLVPEFLPAPEDVETGCEWSAFSCFQKA | | |
| | | QLKSANTGNNERIINVSIKKLKRKPPSTNAGQRQKHTLTCP | | |
| | | SCDSYEKKPPKEFLERFKSLLQKMIHQHLSSRTHGSEDS | | |
| IL-21_5405 | N70G, P107V, | MRSSPGNMERIVICLMVIFLGTLVHKSSSQGQDRHMIRMRQ | 1.554 | 2075 |
| | T110D, T121E | LIDIVDQLKNYVNDLVPEFLPAPEDVETGCEWSAFSCFQKA | | |
| | | QLKSANTGNNERIINVSIKKLKRKVPSDNAGRRQKHRLECP | | |
| | | SCDSYEKKPPKEFLERFKSLLQKMIHQHLSSRTHGSEDS | | |
| IL-21_4972 | T110D, A112P, | MRSSPGNMERIVICLMVIFLGTLVHKSSSQGQDRHMIRMRQ | 1.555 | 2076 |
| | R115G, T121E | LIDIVDQLKNYVNDLVPEFLPAPEDVETNCEWSAFSCFQKA | | |
| | | QLKSANTGNNERIINVSIKKLKRKPPSDNPGRGQKHRLECP | | |
| | | SCDSYEKKPPKEFLERFKSLLQKMIHQHLSSRTHGSEDS | | |
| IL-21_5394 | N70G, P107V, | MRSSPGNMERIVICLMVIFLGTLVHKSSSQGQDRHMIRMRQ | 1.557 | 2077 |
| | R114Q, R119T | LIDIVDQLKNYVNDLVPEFLPAPEDVETGCEWSAFSCFQKA | | |
| | | QLKSANTGNNERIINVSIKKLKRKVPSTNAGQRQKHTLTCP | | |
| | | SCDSYEKKPPKEFLERFKSLLQKMIHQHLSSRTHGSEDS | | |
| IL-21_5589 | P107V, R114G, | MRSSPGNMERIVICLMVIFLGTLVHKSSSQGQDRHMIRMRQ | 1.56 | 2078 |
| | R119T | LIDIVDQLKNYVNDLVPEFLPAPEDVETNCEWSAFSCFQKA | | |
| | | QLKSANTGNNERIINVSIKKLKRKVPSTNAGGRQKHTLTCP | | |
| | | SCDSYEKKPPKEFLERFKSLLQKMIHQHLSSRTHGSEDS | | |
| IL-21_5831 | K81D, A112P | MRSSPGNMERIVICLMVIFLGTLVHKSSSQGQDRHMIRMRQ | 1.563 | 2079 |
| | | LIDIVDQLKNYVNDLVPEFLPAPEDVETNCEWSAFSCFQDA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5712 | N70G, R115G, | MRSSPGNMERIVICLMVIFLGTLVHKSSSQGQDRHMIRMRQ | 1.564 | 2080 |
| | R119T | LIDIVDQLKNYVNDLVPEFLPAPEDVETGCEWSAFSCFQKA | | |
| | | QLKSANTGNNERIINVSIKKLKRKPPSTNAGRGQKHTLTCP | | |
| | | SCDSYEKKPPKEFLERFKSLLQKMIHQHLSSRTHGSEDS | | |
| IL-21_6351 | H35I, P107V, | MRSSPGNMERIVICLMVIFLGTLVHKSSSQGQDRIMIRMRQ | 1.566 | 2081 |
| | R115G | LIDIVDQLKNYVNDLVPEFLPAPEDVETNCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5400 | N70G, P107V, | MRSSPGNMERIVICLMVIFLGTLVHKSSSQGQDRHMIRMRQ | 1.568 | 2082 |
| | A112P, H118D | LIDIVDQLKNYVNDLVPEFLPAPEDVETGCEWSAFSCFQKA | | |
| | | QLKSANTGNNERIINVSIKKLKRKVPSTNPGRRQKDRLTCP | | |
| | | SCDSYEKKPPKEFLERFKSLLQKMIHQHLSSRTHGSEDS | | |
| IL-21_5406 | N70G, P107V, | MRSSPGNMERIVICLMVIFLGTLVHKSSSQGQDRHMIRMRQ | 1.568 | 2083 |
| | T110D, R119T | LIDIVDQLKNYVNDLVPEFLPAPEDVETGCEWSAFSCFQKA | | |
| | | QLKSANTGNNERIINVSIKKLKRKVPSDNAGRRQKHTLTCP | | |
| | | SCDSYEKKPPKEFLERFKSLLQKMIHQHLSSRTHGSEDS | | |
| IL-21_6372 | H35I, N70G, | MRSSPGNMERIVICLMVIFLGTLVHKSSSQGQDRIMIRMRQ | 1.57 | 2084 |
| | R115G | LIDIVDQLKNYVNDLVPEFLPAPEDVETGCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5745 | N70G, P107V, | MRSSPGNMERIVICLMVIFLGTLVHKSSSQGQDRHMIRMRQ | 1.571 | 2085 |
| | R115G | LIDIVDQLKNYVNDLVPEFLPAPEDVETGCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5017 | P107V, A112P, | MRSSPGNMERIVICLMVIFLGTLVHKSSSQGQDRHMIRMRQ | 1.572 | 2086 |
| | R115G, R119T | LIDIVDQLKNYVNDLVPEFLPAPEDVETNCEWSAFSCFQKA | | |
| | | QLKSANTGNNERIINVSIKKLKRKVPSTNPGRGQKHTLTCP | | |
| | | SCDSYEKKPPKEFLERFKSLLQKMIHQHLSSRTHGSEDS | | |
| IL-21_5926 | I45T, N70G, | MRSSPGNMERIVICLMVIFLGTLVHKSSSQGQDRHMIRMRQ | 1.572 | 2087 |
| | T110D, R114G | LIDTVDQLKNYVNDLVPEFLPAPEDVETGCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_6024 | H35F, N70G, | MRSSPGNMERIVICLMVIFLGTLVHKSSSQGQDREMIRMRQ | 1.574 | 2088 |
| | A112P, R115G | LIDIVDQLKNYVNDLVPEFLPAPEDVETGCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_6079 | H35F, I45T, | MRSSPGNMERIVICLMVIFLGTLVHKSSSQGQDREMIRMRQ | 1.575 | 2089 |
| | V98E, P107V | LIDTVDQLKNYVNDLVPEFLPAPEDVETNCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5563 | T110D, R114G, | MRSSPGNMERIVICLMVIFLGTLVHKSSSQGQDRHMIRMRQ | 1.575 | 2090 |
| | R119T | LIDIVDQLKNYVNDLVPEFLPAPEDVETNCEWSAFSCFQKA | | |
| | | QLKSANTGNNERIINVSIKKLKRKPPSDNAGGRQKHTLTCP | | |
| | | SCDSYEKKPPKEFLERFKSLLQKMIHQHLSSRTHGSEDS | | |
| IL-21_6236 | H35I, I45T, | MRSSPGNMERIVICLMVIFLGTLVHKSSSQGQDRIMIRMRQ | 1.576 | 2091 |
| | N70G, R114G | LIDTVDQLKNYVNDLVPEFLPAPEDVETGCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_4936 | A112P, R114Q, | MRSSPGNMERIVICLMVIFLGTLVHKSSSQGQDRHMIRMRQ | 1.577 | 2092 |
| | H118D, T121E | LIDIVDQLKNYVNDLVPEFLPAPEDVETNCEWSAFSCFQKA | | |
| | | QLKSANTGNNERIINVSIKKLKRKPPSTNPGQRQKDRLECP | | |
| | | SCDSYEKKPPKEFLERFKSLLQKMIHQHLSSRTHGSEDS | | |
| IL-21_6244 | I45T, R114G, | MRSSPGNMERIVICLMVIFLGTLVHKSSSQGQDRHMIRMRQ | 1.579 | 2093 |
| | R115D | LIDTVDQLKNYVNDLVPEFLPAPEDVETNCEWSAFSCFQKA | | |
| | | QLKSANTGNNERIINVSIKKLKRKPPSTNAGGDQKHRLTCP | | |
| | | SCDSYEKKPPKEFLERFKSLLQKMIHQHLSSRTHGSEDS | | |
| IL-21_5529 | R114Q, R115G, | MRSSPGNMERIVICLMVIFLGTLVHKSSSQGQDRHMIRMRQ | 1.579 | 2094 |
| | R119T | LIDIVDQLKNYVNDLVPEFLPAPEDVETNCEWSAFSCFQKA | | |
| | | QLKSANTGNNERIINVSIKKLKRKPPSTNAGQGQKHTLTCP | | |
| | | SCDSYEKKPPKEFLERFKSLLQKMIHQHLSSRTHGSEDS | | |
| IL-21_5338 | N70G, A112P, | MRSSPGNMERIVICLMVIFLGTLVHKSSSQGQDRHMIRMRQ | 1.584 | 2095 |
| | R115G, R119T | LIDIVDQLKNYVNDLVPEFLPAPEDVETGCEWSAFSCFQKA | | |
| | | QLKSANTGNNERIINVSIKKLKRKPPSTNPGRGQKHTLTCP | | |
| | | SCDSYEKKPPKEFLERFKSLLQKMIHQHLSSRTHGSEDS | | |
| IL-21_5539 | A112P, R115G, | MRSSPGNMERIVICLMVIFLGTLVHKSSSQGQDRHMIRMRQ | 1.585 | 2096 |
| | H118D | LIDIVDQLKNYVNDLVPEFLPAPEDVETNCEWSAFSCFQKA | | |
| | | QLKSANTGNNERIINVSIKKLKRKPPSTNPGRGQKDRLTCP | | |
| | | SCDSYEKKPPKEFLERFKSLLQKMIHQHLSSRTHGSEDS | | |
| IL-21_6115 | H35I, P107V, | MRSSPGNMERIVICLMVIFLGTLVHKSSSQGQDRIMIRMRQ | 1.586 | 2097 |
| | A112P, R114G | LIDIVDQLKNYVNDLVPEFLPAPEDVETNCEWSAFSCFQKA | | |
| | | QLKSANTGNNERIINVSIKKLKRKVPSTNPGGRQKHRLTCP | | |
| | | SCDSYEKKPPKEFLERFKSLLQKMIHQHLSSRTHGSEDS | | |
| IL-21_5800 | T110D, R115G | MRSSPGNMERIVICLMVIFLGTLVHKSSSQGQDRHMIRMRQ | 1.587 | 2098 |
| | | LIDIVDQLKNYVNDLVPEFLPAPEDVETNCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_5518 | R114G, H118D, | MRSSPGNMERIVICLMVIFLGTLVHKSSSQGQDRHMIRMRQ | 1.588 | 2099 |
| | R119T | LIDIVDQLKNYVNDLVPEFLPAPEDVETNCEWSAFSCFQKA | | |
| | | QLKSANTGNNERIINVSIKKLKRKPPSTNAGGRQKDTLTCP | | |
| | | SCDSYEKKPPKEFLERFKSLLQKMIHQHLSSRTHGSEDS | | |
| IL-21_5373 | N70G, T110D, | MRSSPGNMERIVICLMVIFLGTLVHKSSSQGQDRHMIRMRQ | 1.588 | 2100 |
| | A112P, R119T | LIDIVDQLKNYVNDLVPEFLPAPEDVETGCEWSAFSCFQKA | | |
| | | QLKSANTGNNERIINVSIKKLKRKPPSDNPGRRQKHTLTCP | | |
| | | SCDSYEKKPPKEFLERFKSLLQKMIHQHLSSRTHGSEDS | | |
| IL-21_4926 | A112P, R114G, | MRSSPGNMERIVICLMVIFLGTLVHKSSSQGQDRHMIRMRQ | 1.592 | 2101 |
| | R119T, T121E | LIDIVDQLKNYVNDLVPEFLPAPEDVETNCEWSAFSCFQKA | | |
| | | QLKSANTGNNERIINVSIKKLKRKPPSTNPGGRQKHTLECP | | |
| | | SCDSYEKKPPKEFLERFKSLLQKMIHQHLSSRTHGSEDS | | |
| IL-21_6091 | H35F, I45T, | MRSSPGNMERIVICLMVIFLGTLVHKSSSQGQDREMIRMRQ | 1.593 | 2102 |
| | N70G, R114Q | LIDTVDQLKNYVNDLVPEFLPAPEDVETGCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_6077 | H35F, I45T, | MRSSPGNMERIVICLMVIFLGTLVHKSSSQGQDREMIRMRQ | 1.593 | 2103 |
| | V98E, A112P | LIDTVDQLKNYVNDLVPEFLPAPEDVETNCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5718 | N70G, R114G, | MRSSPGNMERIVICLMVIFLGTLVHKSSSQGQDRHMIRMRQ | 1.594 | 2104 |
| | R119T | LIDIVDQLKNYVNDLVPEFLPAPEDVETGCEWSAFSCFQKA | | |
| | | QLKSANTGNNERIINVSIKKLKRKPPSTNAGGRQKHTLTCP | | |
| | | SCDSYEKKPPKEFLERFKSLLQKMIHQHLSSRTHGSEDS | | |
| IL-21_4938 | A112P, R114Q, | MRSSPGNMERIVICLMVIFLGTLVHKSSSQGQDRHMIRMRQ | 1.595 | 2105 |
| | R115G, T121E | LIDIVDQLKNYVNDLVPEFLPAPEDVETNCEWSAFSCFQKA | | |
| | | QLKSANTGNNERIINVSIKKLKRKPPSTNPGQGQKHRLECP | | |
| | | SCDSYEKKPPKEFLERFKSLLQKMIHQHLSSRTHGSEDS | | |
| IL-21_5732 | N70G, A112P, | MRSSPGNMERIVICLMVIFLGTLVHKSSSQGQDRHMIRMRQ | 1.595 | 2106 |
| | R114G | LIDIVDQLKNYVNDLVPEFLPAPEDVETGCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5564 | T110D, R114G, | MRSSPGNMERIVICLMVIFLGTLVHKSSSQGQDRHMIRMRQ | 1.596 | 2107 |
| | H118D | LIDIVDQLKNYVNDLVPEFLPAPEDVETNCEWSAFSCFQKA | | |
| | | QLKSANTGNNERIINVSIKKLKRKPPSDNAGGRQKDRLTCP | | |
| | | SCDSYEKKPPKEFLERFKSLLQKMIHQHLSSRTHGSEDS | | |
| IL-21_5028 | P107V, A112P, | MRSSPGNMERIVICLMVIFLGTLVHKSSSQGQDRHMIRMRQ | 1.596 | 2108 |
| | R114Q, R119T | LIDIVDQLKNYVNDLVPEFLPAPEDVETNCEWSAFSCFQKA | | |
| | | QLKSANTGNNERIINVSIKKLKRKVPSTNPGQRQKHTLTCP | | |
| | | SCDSYEKKPPKEFLERFKSLLQKMIHQHLSSRTHGSEDS | | |
| IL-21_5540 | A112P, R115D, | MRSSPGNMERIVICLMVIFLGTLVHKSSSQGQDRHMIRMRQ | 1.597 | 2109 |
| | T121E | LIDIVDQLKNYVNDLVPEFLPAPEDVETNCEWSAFSCFQKA | | |
| | | QLKSANTGNNERIINVSIKKLKRKPPSTNPGRDQKHRLECP | | |
| | | SCDSYEKKPPKEFLERFKSLLQKMIHQHLSSRTHGSEDS | | |
| IL-21_5025 | P107V, A112P, | MRSSPGNMERIVICLMVIFLGTLVHKSSSQGQDRHMIRMRQ | 1.599 | 2110 |
| | R114G, R115G | LIDIVDQLKNYVNDLVPEFLPAPEDVETNCEWSAFSCFQKA | | |
| | | QLKSANTGNNERIINVSIKKLKRKVPSTNPGGGQKHRLTCP | | |
| | | SCDSYEKKPPKEFLERFKSLLQKMIHQHLSSRTHGSEDS | | |
| IL-21_4920 | A112P, R115G, | MRSSPGNMERIVICLMVIFLGTLVHKSSSQGQDRHMIRMRQ | 1.606 | 2111 |
| | R119T, T121E | LIDIVDQLKNYVNDLVPEFLPAPEDVETNCEWSAFSCFQKA | | |
| | | QLKSANTGNNERIINVSIKKLKRKPPSTNPGRGQKHTLECP | | |
| | | SCDSYEKKPPKEFLERFKSLLQKMIHQHLSSRTHGSEDS | | |
| IL-21_5030 | P107V, A112P, | MRSSPGNMERIVICLMVIFLGTLVHKSSSQGQDRHMIRMRQ | 1.61 | 2112 |
| | R114Q, R115G | LIDIVDQLKNYVNDLVPEFLPAPEDVETNCEWSAFSCFQKA | | |
| | | QLKSANTGNNERIINVSIKKLKRKVPSTNPGQGQKHRLTCP | | |
| | | SCDSYEKKPPKEFLERFKSLLQKMIHQHLSSRTHGSEDS | | |
| IL-21_5510 | R115G, R119T, | MRSSPGNMERIVICLMVIFLGTLVHKSSSQGQDRHMIRMRQ | 1.614 | 2113 |
| | T121E | LIDIVDQLKNYVNDLVPEFLPAPEDVETNCEWSAFSCFQKA | | |
| | | QLKSANTGNNERIINVSIKKLKRKPPSTNAGRGQKHTLECP | | |
| | | SCDSYEKKPPKEFLERFKSLLQKMIHQHLSSRTHGSEDS | | |
| IL-21_5961 | H35F, T110D, | MRSSPGNMERIVICLMVIFLGTLVHKSSSQGQDREMIRMRQ | 1.614 | 2114 |
| | A112P, R114Q | LIDIVDQLKNYVNDLVPEFLPAPEDVETNCEWSAFSCFQKA | | |
| | | QLKSANTGNNERIINVSIKKLKRKPPSDNPGQRQKHRLTCP | | |
| | | SCDSYEKKPPKEFLERFKSLLQKMIHQHLSSRTHGSEDS | | |
| IL-21_5969 | H35F, P107V, | MRSSPGNMERIVICLMVIFLGTLVHKSSSQGQDREMIRMRQ | 1.621 | 2115 |
| | A112P, R114Q | LIDIVDQLKNYVNDLVPEFLPAPEDVETNCEWSAFSCFQKA | | |
| | | QLKSANTGNNERIINVSIKKLKRKVPSTNPGQRQKHRLTCP | | |
| | | SCDSYEKKPPKEFLERFKSLLQKMIHQHLSSRTHGSEDS | | |
| IL-21_5349 | N70G, A112P, | MRSSPGNMERIVICLMVIFLGTLVHKSSSQGQDRHMIRMRQ | 1.622 | 2116 |
| | R114Q, R119T | LIDIVDQLKNYVNDLVPEFLPAPEDVETGCEWSAFSCFQKA | | |
| | | QLKSANTGNNERIINVSIKKLKRKPPSTNPGQRQKHTLTCP | | |
| | | SCDSYEKKPPKEFLERFKSLLQKMIHQHLSSRTHGSEDS | | |
| IL-21_6381 | H35I, I45T, | MRSSPGNMERIVICLMVIFLGTLVHKSSSQGQDRIMIRMRQ | 1.622 | 2117 |
| | R115G | LIDTVDQLKNYVNDLVPEFLPAPEDVETNCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5948 | I45T, N70G, | MRSSPGNMERIVICLMVIFLGTLVHKSSSQGQDRHMIRMRQ | 1.623 | 2118 |
| | K81D, P107V | LIDTVDQLKNYVNDLVPEFLPAPEDVETGCEWSAFSCFQDA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_6354 | H35I, P107V, | MRSSPGNMERIVICLMVIFLGTLVHKSSSQGQDRIMIRMRQ | 1.632 | 2119 |
| | R114Q | LIDIVDQLKNYVNDLVPEFLPAPEDVETNCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5734 | N70G, T110D, | MRSSPGNMERIVICLMVIFLGTLVHKSSSQGQDRHMIRMRQ | 1.633 | 2120 |
| | T121E | LIDIVDQLKNYVNDLVPEFLPAPEDVETGCEWSAFSCFQKA | | |
| | | QLKSANTGNNERIINVSIKKLKRKPPSDNAGRRQKHRLECP | | |
| | | SCDSYEKKPPKEFLERFKSLLQKMIHQHLSSRTHGSEDS | | |
| IL-21_6251 | I45T, T110D, | MRSSPGNMERIVICLMVIFLGTLVHKSSSQGQDRHMIRMRQ | 1.633 | 2121 |
| | R115G | LIDTVDQLKNYVNDLVPEFLPAPEDVETNCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_4939 | A112P, R114Q, | MRSSPGNMERIVICLMVIFLGTLVHKSSSQGQDRHMIRMRQ | 1.634 | 2122 |
| | R115G, R119T | LIDIVDQLKNYVNDLVPEFLPAPEDVETNCEWSAFSCFQKA | | |
| | | QLKSANTGNNERIINVSIKKLKRKPPSTNPGQGQKHTLTCP | | |
| | | SCDSYEKKPPKEFLERFKSLLQKMIHQHLSSRTHGSEDS | | |
| IL-21_6059 | H35F, I45T, | MRSSPGNMERIVICLMVIFLGTLVHKSSSQGQDREMIRMRQ | 1.635 | 2123 |
| | A112P, R115D | LIDTVDQLKNYVNDLVPEFLPAPEDVETNCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_6283 | I45T, N70G, | MRSSPGNMERIVICLMVIFLGTLVHKSSSQGQDRHMIRMRQ | 1.635 | 2124 |
| | V98E | LIDTVDQLKNYVNDLVPEFLPAPEDVETGCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5345 | N70G, A112P, | MRSSPGNMERIVICLMVIFLGTLVHKSSSQGQDRHMIRMRQ | 1.638 | 2125 |
| | R114G, H118D | LIDIVDQLKNYVNDLVPEFLPAPEDVETGCEWSAFSCFQKA | | |
| | | QLKSANTGNNERIINVSIKKLKRKPPSTNPGGRQKDRLTCP | | |
| | | SCDSYEKKPPKEFLERFKSLLQKMIHQHLSSRTHGSEDS | | |
| IL-21_5610 | P107V, T110D, | MRSSPGNMERIVICLMVIFLGTLVHKSSSQGQDRHMIRMRQ | 1.639 | 2126 |
| | R114Q | LIDIVDQLKNYVNDLVPEFLPAPEDVETNCEWSAFSCFQKA | | |
| | | QLKSANTGNNERIINVSIKKLKRKVPSDNAGQRQKHRLTCP | | |
| | | SCDSYEKKPPKEFLERFKSLLQKMIHQHLSSRTHGSEDS | | |
| IL-21_6340 | H35I, R114Q, | MRSSPGNMERIVICLMVIFLGTLVHKSSSQGQDRIMIRMRQ | 1.64 | 2127 |
| | R115G | LIDIVDQLKNYVNDLVPEFLPAPEDVETNCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_6289 | H35F, A112P, | MRSSPGNMERIVICLMVIFLGTLVHKSSSQGQDRFMIRMRQ | 1.641 | 2128 |
| | R115G | LIDIVDQLKNYVNDLVPEFLPAPEDVETNCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5525 | R114Q, R119T, | MRSSPGNMERIVICLMVIFLGTLVHKSSSQGQDRHMIRMRQ | 1.642 | 2129 |
| | T121E | LIDIVDQLKNYVNDLVPEFLPAPEDVETNCEWSAFSCFQKA | | |
| | | QLKSANTGNNERIINVSIKKLKRKPPSTNAGQRQKHTLECP | | |
| | | SCDSYEKKPPKEFLERFKSLLQKMIHQHLSSRTHGSEDS | | |
| IL-21_5653 | V98E, P107V, | MRSSPGNMERIVICLMVIFLGTLVHKSSSQGQDRHMIRMRQ | 1.643 | 2130 |
| | A112P | LIDIVDQLKNYVNDLVPEFLPAPEDVETNCEWSAFSCFQKA | | |
| | | QLKSANTGNNERIINESIKKLKRKVPSTNPGRRQKHRLTCP | | |
| | | SCDSYEKKPPKEFLERFKSLLQKMIHQHLSSRTHGSEDS | | |
| IL-21_5888 | I45T, V98E, | MRSSPGNMERIVICLMVIFLGTLVHKSSSQGQDRHMIRMRQ | 1.643 | 2131 |
| | P107V, A112P | LIDTVDQLKNYVNDLVPEFLPAPEDVETNCEWSAFSCFQKA | | |
| | | QLKSANTGNNERIINESIKKLKRKVPSTNPGRRQKHRLTCP | | |
| | | SCDSYEKKPPKEFLERFKSLLQKMIHQHLSSRTHGSEDS | | |
| IL-21_6070 | H35F, I45T, | MRSSPGNMERIVICLMVIFLGTLVHKSSSQGQDRFMIRMRQ | 1.646 | 2132 |
| | P107V, R114Q | LIDTVDQLKNYVNDLVPEFLPAPEDVETNCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5583 | P107V, R115G, | MRSSPGNMERIVICLMVIFLGTLVHKSSSQGQDRHMIRMRQ | 1.648 | 2133 |
| | R119T | LIDIVDQLKNYVNDLVPEFLPAPEDVETNCEWSAFSCFQKA | | |
| | | QLKSANTGNNERIINVSIKKLKRKVPSTNAGRGQKHTLTCP | | |
| | | SCDSYEKKPPKEFLERFKSLLQKMIHQHLSSRTHGSEDS | | |
| IL-21_5839 | N70G, R115D | MRSSPGNMERIVICLMVIFLGTLVHKSSSQGQDRHMIRMRQ | 1.649 | 2134 |
| | | LIDIVDQLKNYVNDLVPEFLPAPEDVETGCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5939 | I45T, N70G, | MRSSPGNMERIVICLMVIFLGTLVHKSSSQGQDRHMIRMRQ | 1.65 | 2135 |
| | V98E, A112P | LIDTVDQLKNYVNDLVPEFLPAPEDVETGCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5605 | P107V, T110D, | MRSSPGNMERIVICLMVIFLGTLVHKSSSQGQDRHMIRMRQ | 1.651 | 2136 |
| | R119T | LIDIVDQLKNYVNDLVPEFLPAPEDVETNCEWSAFSCFQKA | | |
| | | QLKSANTGNNERIINVSIKKLKRKVPSDNAGRRQKHTLTCP | | |
| | | SCDSYEKKPPKEFLERFKSLLQKMIHQHLSSRTHGSEDS | | |
| IL-21_6097 | H35I, A112P, | MRSSPGNMERIVICLMVIFLGTLVHKSSSQGQDRIMIRMRQ | 1.652 | 2137 |
| | R114G, R115G | LIDIVDQLKNYVNDLVPEFLPAPEDVETNCEWSAFSCFQKA | | |
| | | QLKSANTGNNERIINVSIKKLKRKPPSTNPGGGQKHRLTCP | | |
| | | SCDSYEKKPPKEFLERFKSLLQKMIHQHLSSRTHGSEDS | | |
| IL-21_5018 | P107V, A112P, | MRSSPGNMERIVICLMVIFLGTLVHKSSSQGQDRHMIRMRQ | 1.652 | 2138 |
| | R115G, H118D | LIDIVDQLKNYVNDLVPEFLPAPEDVETNCEWSAFSCFQKA | | |
| | | QLKSANTGNNERIINVSIKKLKRKVPSTNPGRGQKDRLTCP | | |
| | | SCDSYEKKPPKEFLERFKSLLQKMIHQHLSSRTHGSEDS | | |
| IL-21_5057 | P107V, T110D, | MRSSPGNMERIVICLMVIFLGTLVHKSSSQGQDRHMIRMRQ | 1.653 | 2139 |
| | A112P, R114Q | LIDIVDQLKNYVNDLVPEFLPAPEDVETNCEWSAFSCFQKA | | |
| | | QLKSANTGNNERIINVSIKKLKRKVPSDNPGQRQKHRLTCP | | |
| | | SCDSYEKKPPKEFLERFKSLLQKMIHQHLSSRTHGSEDS | | |
| IL-21_6107 | H35I, T110D, | MRSSPGNMERIVICLMVIFLGTLVHKSSSQGQDRIMIRMRQ | 1.653 | 2140 |
| | A112P, R114G | LIDIVDQLKNYVNDLVPEFLPAPEDVETNCEWSAFSCFQKA | | |
| | | QLKSANTGNNERIINVSIKKLKRKPPSDNPGGRQKHRLTCP | | |
| | | SCDSYEKKPPKEFLERFKSLLQKMIHQHLSSRTHGSEDS | | |
| IL-21_6377 | H35I, N70G, | MRSSPGNMERIVICLMVIFLGTLVHKSSSQGQDRIMIRMRQ | 1.655 | 2141 |
| | T110D | LIDIVDQLKNYVNDLVPEFLPAPEDVETGCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5528 | R114Q, R115G, | MRSSPGNMERIVICLMVIFLGTLVHKSSSQGQDRHMIRMRQ | 1.656 | 2142 |
| | T121E | LIDIVDQLKNYVNDLVPEFLPAPEDVETNCEWSAFSCFQKA | | |
| | | QLKSANTGNNERIINVSIKKLKRKPPSTNAGQGQKHRLECP | | |
| | | SCDSYEKKPPKEFLERFKSLLQKMIHQHLSSRTHGSEDS | | |
| IL-21_5553 | T110D, R119T, | MRSSPGNMERIVICLMVIFLGTLVHKSSSQGQDRHMIRMRQ | 1.657 | 2143 |
| | T121E | LIDIVDQLKNYVNDLVPEFLPAPEDVETNCEWSAFSCFQKA | | |
| | | QLKSANTGNNERIINVSIKKLKRKPPSDNAGRRQKHTLECP | | |
| | | SCDSYEKKPPKEFLERFKSLLQKMIHQHLSSRTHGSEDS | | |
| IL-21_5788 | R114Q, R115G | MRSSPGNMERIVICLMVIFLGTLVHKSSSQGQDRHMIRMRQ | 1.658 | 2144 |
| | | LIDIVDQLKNYVNDLVPEFLPAPEDVETNCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_6200 | H35I, I45T, | MRSSPGNMERIVICLMVIFLGTLVHKSSSQGQDRIMIRMRQ | 1.658 | 2145 |
| | R114G, R115G | LIDTVDQLKNYVNDLVPEFLPAPEDVETNCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5377 | N70G, T110D, | MRSSPGNMERIVICLMVIFLGTLVHKSSSQGQDRHMIRMRQ | 1.66 | 2146 |
| | A112P, R114G | LIDIVDQLKNYVNDLVPEFLPAPEDVETGCEWSAFSCFQKA | | |
| | | QLKSANTGNNERIINVSIKKLKRKPPSDNPGGRQKHRLTCP | | |
| | | SCDSYEKKPPKEFLERFKSLLQKMIHQHLSSRTHGSEDS | | |
| IL-21_5711 | N70G, R115G, | MRSSPGNMERIVICLMVIFLGTLVHKSSSQGQDRHMIRMRQ | 1.662 | 2147 |
| | T121E | LIDIVDQLKNYVNDLVPEFLPAPEDVETGCEWSAFSCFQKA | | |
| | | QLKSANTGNNERIINVSIKKLKRKPPSTNAGRGQKHRLECP | | |
| | | SCDSYEKKPPKEFLERFKSLLQKMIHQHLSSRTHGSEDS | | |
| IL-21_6324 | H35F, N70G, | MRSSPGNMERIVICLMVIFLGTLVHKSSSQGQDRFMIRMRQ | 1.662 | 2148 |
| | T110D | LIDIVDQLKNYVNDLVPEFLPAPEDVETGCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5545 | A112P, R114G, | MRSSPGNMERIVICLMVIFLGTLVHKSSSQGQDRHMIRMRQ | 1.663 | 2149 |
| | H118D | LIDIVDQLKNYVNDLVPEFLPAPEDVETNCEWSAFSCFQKA | | |
| | | QLKSANTGNNERIINVSIKKLKRKPPSTNPGGRQKDRLTCP | | |
| | | SCDSYEKKPPKEFLERFKSLLQKMIHQHLSSRTHGSEDS | | |
| IL-21_6349 | H35I, T110D, | MRSSPGNMERIVICLMVIFLGTLVHKSSSQGQDRIMIRMRQ | 1.664 | 2150 |
| | R114Q | LIDIVDQLKNYVNDLVPEFLPAPEDVETNCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_6291 | H35F, A112P, | MRSSPGNMERIVICLMVIFLGTLVHKSSSQGQDREMIRMRQ | 1.665 | 2151 |
| | R114G | LIDIVDQLKNYVNDLVPEFLPAPEDVETNCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_6088 | H35F, I45T, | MRSSPGNMERIVICLMVIFLGTLVHKSSSQGQDRFMIRMRQ | 1.665 | 2152 |
| | N70G, R115G | LIDTVDQLKNYVNDLVPEFLPAPEDVETGCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5602 | P107V, A112P, | MRSSPGNMERIVICLMVIFLGTLVHKSSSQGQDRHMIRMRQ | 1.666 | 2153 |
| | R114G | LIDIVDQLKNYVNDLVPEFLPAPEDVETNCEWSAFSCFQKA | | |
| | | QLKSANTGNNERIINVSIKKLKRKVPSTNPGGRQKHRLTCP | | |
| | | SCDSYEKKPPKEFLERFKSLLQKMIHQHLSSRTHGSEDS | | |
| IL-21_5520 | R114G, R115G, | MRSSPGNMERIVICLMVIFLGTLVHKSSSQGQDRHMIRMRQ | 1.666 | 2154 |
| | R119T | LIDIVDQLKNYVNDLVPEFLPAPEDVETNCEWSAFSCFQKA | | |
| | | QLKSANTGNNERIINVSIKKLKRKPPSTNAGGGQKHTLTCP | | |
| | | SCDSYEKKPPKEFLERFKSLLQKMIHQHLSSRTHGSEDS | | |
| IL-21_5783 | R114G, R115G | MRSSPGNMERIVICLMVIFLGTLVHKSSSQGQDRHMIRMRQ | 1.667 | 2155 |
| | | LIDIVDQLKNYVNDLVPEFLPAPEDVETNCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_6237 | H35I, I45T, | MRSSPGNMERIVICLMVIFLGTLVHKSSSQGQDRIMIRMRQ | 1.667 | 2156 |
| | N70G, R114Q | LIDTVDQLKNYVNDLVPEFLPAPEDVETGCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_6292 | H35F, A112P, | MRSSPGNMERIVICLMVIFLGTLVHKSSSQGQDREMIRMRQ | 1.668 | 2157 |
| | R114Q | LIDIVDQLKNYVNDLVPEFLPAPEDVETNCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5379 | N70G, P107V, | MRSSPGNMERIVICLMVIFLGTLVHKSSSQGQDRHMIRMRQ | 1.674 | 2158 |
| | R119T, T121E | LIDIVDQLKNYVNDLVPEFLPAPEDVETGCEWSAFSCFQKA | | |
| | | QLKSANTGNNERIINVSIKKLKRKVPSTNAGRRQKHTLECP | | |
| | | SCDSYEKKPPKEFLERFKSLLQKMIHQHLSSRTHGSEDS | | |
| IL-21_6353 | H35I, P107V, | MRSSPGNMERIVICLMVIFLGTLVHKSSSQGQDRIMIRMRQ | 1.677 | 2159 |
| | R114G | LIDIVDQLKNYVNDLVPEFLPAPEDVETNCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5582 | P107V, R115G, | MRSSPGNMERIVICLMVIFLGTLVHKSSSQGQDRHMIRMRQ | 1.678 | 2160 |
| | T121E | LIDIVDQLKNYVNDLVPEFLPAPEDVETNCEWSAFSCFQKA | | |
| | | QLKSANTGNNERIINVSIKKLKRKVPSTNAGRGQKHRLECP | | |
| | | SCDSYEKKPPKEFLERFKSLLQKMIHQHLSSRTHGSEDS | | |
| IL-21_6328 | H35F, I45T, | MRSSPGNMERIVICLMVIFLGTLVHKSSSQGQDREMIRMRQ | 1.685 | 2161 |
| | R115G | LIDTVDQLKNYVNDLVPEFLPAPEDVETNCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_6069 | H35F, I45T, | MRSSPGNMERIVICLMVIFLGTLVHKSSSQGQDREMIRMRQ | 1.685 | 2162 |
| | P107V, R114G | LIDTVDQLKNYVNDLVPEFLPAPEDVETNCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_6399 | I45T, K81D | MRSSPGNMERIVICLMVIFLGTLVHKSSSQGQDRHMIRMRQ | 1.686 | 2163 |
| | | LIDTVDQLKNYVNDLVPEFLPAPEDVETNCEWSAFSCFQDA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6206 | H35I, I45T, | MRSSPGNMERIVICLMVIFLGTLVHKSSSQGQDRIMIRMRQ | 1.687 | 2164 |
| | A112P, R114G | LIDTVDQLKNYVNDLVPEFLPAPEDVETNCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5574 | T110D, A112P, | MRSSPGNMERIVICLMVIFLGTLVHKSSSQGQDRHMIRMRQ | 1.688 | 2165 |
| | H118D | LIDIVDQLKNYVNDLVPEFLPAPEDVETNCEWSAFSCFQKA | | |
| | | QLKSANTGNNERIINVSIKKLKRKPPSDNPGRRQKDRLTCP | | |
| | | SCDSYEKKPPKEFLERFKSLLQKMIHQHLSSRTHGSEDS | | |
| IL-21_6356 | H35I, P107V, | MRSSPGNMERIVICLMVIFLGTLVHKSSSQGQDRIMIRMRQ | 1.688 | 2166 |
| | T110D | LIDIVDQLKNYVNDLVPEFLPAPEDVETNCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_4929 | A112P, R114G, | MRSSPGNMERIVICLMVIFLGTLVHKSSSQGQDRHMIRMRQ | 1.688 | 2167 |
| | R115G, T121E | LIDIVDQLKNYVNDLVPEFLPAPEDVETNCEWSAFSCFQKA | | |
| | | QLKSANTGNNERIINVSIKKLKRKPPSTNPGGGQKHRLECP | | |
| | | SCDSYEKKPPKEFLERFKSLLQKMIHQHLSSRTHGSEDS | | |
| IL-21_6108 | H35I, T110D, | MRSSPGNMERIVICLMVIFLGTLVHKSSSQGQDRIMIRMRQ | 1.689 | 2168 |
| | A112P, R114Q | LIDIVDQLKNYVNDLVPEFLPAPEDVETNCEWSAFSCFQKA | | |
| | | QLKSANTGNNERIINVSIKKLKRKPPSDNPGQRQKHRLTCP | | |
| | | SCDSYEKKPPKEFLERFKSLLQKMIHQHLSSRTHGSEDS | | |
| IL-21_5052 | P107V, T110D, | MRSSPGNMERIVICLMVIFLGTLVHKSSSQGQDRHMIRMRQ | 1.692 | 2169 |
| | A112P, R119T | LIDIVDQLKNYVNDLVPEFLPAPEDVETNCEWSAFSCFQKA | | |
| | | QLKSANTGNNERIINVSIKKLKRKVPSDNPGRRQKHTLTCP | | |
| | | SCDSYEKKPPKEFLERFKSLLQKMIHQHLSSRTHGSEDS | | |
| IL-21_6121 | H35I, P107V, | MRSSPGNMERIVICLMVIFLGTLVHKSSSQGQDRIMIRMRQ | 1.692 | 2170 |
| | T110D, A112P | LIDIVDQLKNYVNDLVPEFLPAPEDVETNCEWSAFSCFQKA | | |
| | | QLKSANTGNNERIINVSIKKLKRKVPSDNPGRRQKHRLTCP | | |
| | | SCDSYEKKPPKEFLERFKSLLQKMIHQHLSSRTHGSEDS | | |
| IL-21_6406 | H35F, T110D | MRSSPGNMERIVICLMVIFLGTLVHKSSSQGQDREMIRMRQ | 1.693 | 2171 |
| | | LIDIVDQLKNYVNDLVPEFLPAPEDVETNCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_6268 | I45T, V98E, | MRSSPGNMERIVICLMVIFLGTLVHKSSSQGQDRHMIRMRQ | 1.695 | 2172 |
| | P107V | LIDTVDQLKNYVNDLVPEFLPAPEDVETNCEWSAFSCFQKA | | |
| | | QLKSANTGNNERIINESIKKLKRKVPSTNAGRRQKHRLTCP | | |
| | | SCDSYEKKPPKEFLERFKSLLQKMIHQHLSSRTHGSEDS | | |
| IL-21_5722 | N70G, R114Q, | MRSSPGNMERIVICLMVIFLGTLVHKSSSQGQDRHMIRMRQ | 1.695 | 2173 |
| | T121E | LIDIVDQLKNYVNDLVPEFLPAPEDVETGCEWSAFSCFQKA | | |
| | | QLKSANTGNNERIINVSIKKLKRKPPSTNAGQRQKHRLECP | | |
| | | SCDSYEKKPPKEFLERFKSLLQKMIHQHLSSRTHGSEDS | | |
| IL-21_5866 | I45T, P107V, | MRSSPGNMERIVICLMVIFLGTLVHKSSSQGQDRHMIRMRQ | 1.695 | 2174 |
| | T110D, R115G | LIDTVDQLKNYVNDLVPEFLPAPEDVETNCEWSAFSCFQKA | | |
| | | QLKSANTGNNERIINVSIKKLKRKVPSDNAGRGQKHRLTCP | | |
| | | SCDSYEKKPPKEFLERFKSLLQKMIHQHLSSRTHGSEDS | | |
| IL-21_6239 | H351, I45T, | MRSSPGNMERIVICLMVIFLGTLVHKSSSQGQDRIMIRMRQ | 1.696 | 2175 |
| | N70G, T110D | LIDTVDQLKNYVNDLVPEFLPAPEDVETGCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5750 | N70G, P107V, | MRSSPGNMERIVICLMVIFLGTLVHKSSSQGQDRHMIRMRQ | 1.696 | 2176 |
| | T110D | LIDIVDQLKNYVNDLVPEFLPAPEDVETGCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5594 | P107V, R114Q, | MRSSPGNMERIVICLMVIFLGTLVHKSSSQGQDRHMIRMRQ | 1.696 | 2177 |
| | R119T | LIDIVDQLKNYVNDLVPEFLPAPEDVETNCEWSAFSCFQKA | | |
| | | QLKSANTGNNERIINVSIKKLKRKVPSTNAGQRQKHTLTCP | | |
| | | SCDSYEKKPPKEFLERFKSLLQKMIHQHLSSRTHGSEDS | | |
| IL-21_5717 | N70G, R114G, | MRSSPGNMERIVICLMVIFLGTLVHKSSSQGQDRHMIRMRQ | 1.698 | 2178 |
| | T121E | LIDIVDQLKNYVNDLVPEFLPAPEDVETGCEWSAFSCFQKA | | |
| | | QLKSANTGNNERIINVSIKKLKRKPPSTNAGGRQKHRLECP | | |
| | | SCDSYEKKPPKEFLERFKSLLQKMIHQHLSSRTHGSEDS | | |
| IL-21_6113 | H35I, P107V, | MRSSPGNMERIVICLMVIFLGTLVHKSSSQGQDRIMIRMRQ | 1.7 | 2179 |
| | A112P, R115G | LIDIVDQLKNYVNDLVPEFLPAPEDVETNCEWSAFSCFQKA | | |
| | | QLKSANTGNNERIINVSIKKLKRKVPSTNPGRGQKHRLTCP | | |
| | | SCDSYEKKPPKEFLERFKSLLQKMIHQHLSSRTHGSEDS | | |
| IL-21_5931 | I45T, N70G, | MRSSPGNMERIVICLMVIFLGTLVHKSSSQGQDRHMIRMRQ | 1.704 | 2180 |
| | P107V, R114G | LIDTVDQLKNYVNDLVPEFLPAPEDVETGCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5842 | N70G, T110D | MRSSPGNMERIVICLMVIFLGTLVHKSSSQGQDRHMIRMRQ | 1.705 | 2181 |
| | | LIDIVDQLKNYVNDLVPEFLPAPEDVETGCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5014 | P107V, A112P, | MRSSPGNMERIVICLMVIFLGTLVHKSSSQGQDRHMIRMRQ | 1.705 | 2182 |
| | H118D, T121E | LIDIVDQLKNYVNDLVPEFLPAPEDVETNCEWSAFSCFQKA | | |
| | | QLKSANTGNNERIINVSIKKLKRKVPSTNPGRRQKDRLECP | | |
| | | SCDSYEKKPPKEFLERFKSLLQKMIHQHLSSRTHGSEDS | | |
| IL-21_5567 | T110D, R114Q, | MRSSPGNMERIVICLMVIFLGTLVHKSSSQGQDRHMIRMRQ | 1.706 | 2183 |
| | T121E | LIDIVDQLKNYVNDLVPEFLPAPEDVETNCEWSAFSCFQKA | | |
| | | QLKSANTGNNERIINVSIKKLKRKPPSDNAGQRQKHRLECP | | |
| | | SCDSYEKKPPKEFLERFKSLLQKMIHQHLSSRTHGSEDS | | |
| IL-21_6378 | H35I, N70G, | MRSSPGNMERIVICLMVIFLGTLVHKSSSQGQDRIMIRMRQ | 1.707 | 2184 |
| | P107V | LIDIVDQLKNYVNDLVPEFLPAPEDVETGCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5554 | T110D, H118D, | MRSSPGNMERIVICLMVIFLGTLVHKSSSQGQDRHMIRMRQ | 1.709 | 2185 |
| | T121E | LIDIVDQLKNYVNDLVPEFLPAPEDVETNCEWSAFSCFQKA | | |
| | | QLKSANTGNNERIINVSIKKLKRKPPSDNAGRRQKDRLECP | | |
| | | SCDSYEKKPPKEFLERFKSLLQKMIHQHLSSRTHGSEDS | | |
| IL-21_5802 | T110D, R114G | MRSSPGNMERIVICLMVIFLGTLVHKSSSQGQDRHMIRMRQ | 1.711 | 2186 |
| | | LIDIVDQLKNYVNDLVPEFLPAPEDVETNCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5557 | T110D, R115G, | MRSSPGNMERIVICLMVIFLGTLVHKSSSQGQDRHMIRMRQ | 1.711 | 2187 |
| | R119T | LIDIVDQLKNYVNDLVPEFLPAPEDVETNCEWSAFSCFQKA | | |
| | | QLKSANTGNNERIINVSIKKLKRKPPSDNAGRGQKHTLTCP | | |
| | | SCDSYEKKPPKEFLERFKSLLQKMIHQHLSSRTHGSEDS | | |
| IL-21_5846 | I45T, A112P, | MRSSPGNMERIVICLMVIFLGTLVHKSSSQGQDRHMIRMRQ | 1.711 | 2188 |
| | R114G, R115G | LIDTVDQLKNYVNDLVPEFLPAPEDVETNCEWSAFSCFQKA | | |
| | | QLKSANTGNNERIINVSIKKLKRKPPSTNPGGGQKHRLTCP | | |
| | | SCDSYEKKPPKEFLERFKSLLQKMIHQHLSSRTHGSEDS | | |
| IL-21_5864 | I45T, P107V, | MRSSPGNMERIVICLMVIFLGTLVHKSSSQGQDRHMIRMRQ | 1.713 | 2189 |
| | A112P, R114G | LIDTVDQLKNYVNDLVPEFLPAPEDVETNCEWSAFSCFQKA | | |
| | | QLKSANTGNNERIINVSIKKLKRKVPSTNPGGRQKHRLTCP | | |
| | | SCDSYEKKPPKEFLERFKSLLQKMIHQHLSSRTHGSEDS | | |
| IL-21_6404 | H35F, R114Q | MRSSPGNMERIVICLMVIFLGTLVHKSSSQGQDREMIRMRQ | 1.714 | 2190 |
| | | LIDIVDQLKNYVNDLVPEFLPAPEDVETNCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5778 | R115D, R119T | MRSSPGNMERIVICLMVIFLGTLVHKSSSQGQDRHMIRMRQ | 1.719 | 2191 |
| | | LIDIVDQLKNYVNDLVPEFLPAPEDVETNCEWSAFSCFQKA | | |
| | | QLKSANTGNNERIINVSIKKLKRKPPSTNAGRDQKHTLTCP | | |
| | | SCDSYEKKPPKEFLERFKSLLQKMIHQHLSSRTHGSEDS | | |
| IL-21_6032 | H35F, N70G, | MRSSPGNMERIVICLMVIFLGTLVHKSSSQGQDREMIRMRQ | 1.721 | 2192 |
| | T110D, A112P | LIDIVDQLKNYVNDLVPEFLPAPEDVETGCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5016 | P107V, A112P, | MRSSPGNMERIVICLMVIFLGTLVHKSSSQGQDRHMIRMRQ | 1.723 | 2193 |
| | R115G, T121E | LIDIVDQLKNYVNDLVPEFLPAPEDVETNCEWSAFSCFQKA | | |
| | | QLKSANTGNNERIINVSIKKLKRKVPSTNPGRGQKHRLECP | | |
| | | SCDSYEKKPPKEFLERFKSLLQKMIHQHLSSRTHGSEDS | | |
| IL-21_5854 | I45T, T110D, | MRSSPGNMERIVICLMVIFLGTLVHKSSSQGQDRHMIRMRQ | 1.724 | 2194 |
| | A112P, R115G | LIDTVDQLKNYVNDLVPEFLPAPEDVETNCEWSAFSCFQKA | | |
| | | QLKSANTGNNERIINVSIKKLKRKPPSDNPGRGQKHRLTCP | | |
| | | SCDSYEKKPPKEFLERFKSLLQKMIHQHLSSRTHGSEDS | | |
| IL-21_5735 | N70G, T110D, | MRSSPGNMERIVICLMVIFLGTLVHKSSSQGQDRHMIRMRQ | 1.725 | 2195 |
| | R119T | LIDIVDQLKNYVNDLVPEFLPAPEDVETGCEWSAFSCFQKA | | |
| | | QLKSANTGNNERIINVSIKKLKRKPPSDNAGRRQKHTLTCP | | |
| | | SCDSYEKKPPKEFLERFKSLLQKMIHQHLSSRTHGSEDS | | |
| IL-21_6277 | I45T, N70G, | MRSSPGNMERIVICLMVIFLGTLVHKSSSQGQDRHMIRMRQ | 1.726 | 2196 |
| | R115D | LIDTVDQLKNYVNDLVPEFLPAPEDVETGCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_4935 | A112P, R114Q, | MRSSPGNMERIVICLMVIFLGTLVHKSSSQGQDRHMIRMRQ | 1.729 | 2197 |
| | R119T, T121E | LIDIVDQLKNYVNDLVPEFLPAPEDVETNCEWSAFSCFQKA | | |
| | | QLKSANTGNNERIINVSIKKLKRKPPSTNPGQRQKHTLECP | | |
| | | SCDSYEKKPPKEFLERFKSLLQKMIHQHLSSRTHGSEDS | | |
| IL-21_5782 | R114G, H118D | MRSSPGNMERIVICLMVIFLGTLVHKSSSQGQDRHMIRMRQ | 1.731 | 2198 |
| | | LIDIVDQLKNYVNDLVPEFLPAPEDVETNCEWSAFSCFQKA | | |
| | | QLKSANTGNNERIINVSIKKLKRKPPSTNAGGRQKDRLTCP | | |
| | | SCDSYEKKPPKEFLERFKSLLQKMIHQHLSSRTHGSEDS | | |
| IL-21_5579 | P107V, R119T, | MRSSPGNMERIVICLMVIFLGTLVHKSSSQGQDRHMIRMRQ | 1.731 | 2199 |
| | T121E | LIDIVDQLKNYVNDLVPEFLPAPEDVETNCEWSAFSCFQKA | | |
| | | QLKSANTGNNERIINVSIKKLKRKVPSTNAGRRQKHTLECP | | |
| | | SCDSYEKKPPKEFLERFKSLLQKMIHQHLSSRTHGSEDS | | |
| IL-21_5968 | H35F, P107V, | MRSSPGNMERIVICLMVIFLGTLVHKSSSQGQDREMIRMRQ | 1.732 | 2200 |
| | A112P, R114G | LIDIVDQLKNYVNDLVPEFLPAPEDVETNCEWSAFSCFQKA | | |
| | | QLKSANTGNNERIINVSIKKLKRKVPSTNPGGRQKHRLTCP | | |
| | | SCDSYEKKPPKEFLERFKSLLQKMIHQHLSSRTHGSEDS | | |
| IL-21_6253 | I45T, T110D, | MRSSPGNMERIVICLMVIFLGTLVHKSSSQGQDRHMIRMRQ | 1.732 | 2201 |
| | R114G | LIDTVDQLKNYVNDLVPEFLPAPEDVETNCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_6245 | I45T, R114Q, | MRSSPGNMERIVICLMVIFLGTLVHKSSSQGQDRHMIRMRQ | 1.733 | 2202 |
| | R115G | LIDTVDQLKNYVNDLVPEFLPAPEDVETNCEWSAFSCFQKA | | |
| | | QLKSANTGNNERIINVSIKKLKRKPPSTNAGQGQKHRLTCP | | |
| | | SCDSYEKKPPKEFLERFKSLLQKMIHQHLSSRTHGSEDS | | |
| IL-21_5932 | I45T, N70G, | MRSSPGNMERIVICLMVIFLGTLVHKSSSQGQDRHMIRMRQ | 1.739 | 2203 |
| | P107V, R114Q | LIDTVDQLKNYVNDLVPEFLPAPEDVETGCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5848 | I45T, A112P, | MRSSPGNMERIVICLMVIFLGTLVHKSSSQGQDRHMIRMRQ | 1.742 | 2204 |
| | R114Q, R115G | LIDTVDQLKNYVNDLVPEFLPAPEDVETNCEWSAFSCFQKA | | |
| | | QLKSANTGNNERIINVSIKKLKRKPPSTNPGQGQKHRLTCP | | |
| | | SCDSYEKKPPKEFLERFKSLLQKMIHQHLSSRTHGSEDS | | |
| IL-21_6346 | H35I, T110D, | MRSSPGNMERIVICLMVIFLGTLVHKSSSQGQDRIMIRMRQ | 1.745 | 2205 |
| | R115G | LIDIVDQLKNYVNDLVPEFLPAPEDVETNCEWSAFSCFQKA | | |
| | | QLKSANTGNNERIINVSIKKLKRKPPSDNAGRGQKHRLTCP | | |
| | | SCDSYEKKPPKEFLERFKSLLQKMIHQHLSSRTHGSEDS | | |
| IL-21_5856 | I45T, T110D, | MRSSPGNMERIVICLMVIFLGTLVHKSSSQGQDRHMIRMRQ | 1.751 | 2206 |
| | A112P, R114G | LIDTVDQLKNYVNDLVPEFLPAPEDVETNCEWSAFSCFQKA | | |
| | | QLKSANTGNNERIINVSIKKLKRKPPSDNPGGRQKHRLTCP | | |
| | | SCDSYEKKPPKEFLERFKSLLQKMIHQHLSSRTHGSEDS | | |
| IL-21_5777 | R115G, H118D | MRSSPGNMERIVICLMVIFLGTLVHKSSSQGQDRHMIRMRQ | 1.752 | 2207 |
| | | LIDIVDQLKNYVNDLVPEFLPAPEDVETNCEWSAFSCFQKA | | |
| | | QLKSANTGNNERIINVSIKKLKRKPPSTNAGRGQKDRLTCP | | |
| | | SCDSYEKKPPKEFLERFKSLLQKMIHQHLSSRTHGSEDS | | |
| IL-21_5840 | N70G, R114Q | MRSSPGNMERIVICLMVIFLGTLVHKSSSQGQDRHMIRMRQ | 1.752 | 2208 |
| | | LIDIVDQLKNYVNDLVPEFLPAPEDVETGCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5929 | I45T, N70G, | MRSSPGNMERIVICLMVIFLGTLVHKSSSQGQDRHMIRMRQ | 1.753 | 2209 |
| | P107V, R115G | LIDTVDQLKNYVNDLVPEFLPAPEDVETGCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_6209 | H35I, I45T, | MRSSPGNMERIVICLMVIFLGTLVHKSSSQGQDRIMIRMRQ | 1.753 | 2210 |
| | T110D, R115D | LIDTVDQLKNYVNDLVPEFLPAPEDVETNCEWSAFSCFQKA | | |
| | | QLKSANTGNNERIINVSIKKLKRKPPSDNAGRDQKHRLTCP | | |
| | | SCDSYEKKPPKEFLERFKSLLQKMIHQHLSSRTHGSEDS | | |
| IL-21_6348 | H35I, T110D, | MRSSPGNMERIVICLMVIFLGTLVHKSSSQGQDRIMIRMRQ | 1.755 | 2211 |
| | R114G | LIDIVDQLKNYVNDLVPEFLPAPEDVETNCEWSAFSCFQKA | | |
| | | QLKSANTGNNERIINVSIKKLKRKPPSDNAGGRQKHRLTCP | | |
| | | SCDSYEKKPPKEFLERFKSLLQKMIHQHLSSRTHGSEDS | | |
| IL-21_5546 | A112P, R114G, | MRSSPGNMERIVICLMVIFLGTLVHKSSSQGQDRHMIRMRQ | 1.755 | 2212 |
| | R115G | LIDIVDQLKNYVNDLVPEFLPAPEDVETNCEWSAFSCFQKA | | |
| | | QLKSANTGNNERIINVSIKKLKRKPPSTNPGGGQKHRLTCP | | |
| | | SCDSYEKKPPKEFLERFKSLLQKMIHQHLSSRTHGSEDS | | |
| IL-21_5562 | T110D, R114G, | MRSSPGNMERIVICLMVIFLGTLVHKSSSQGQDRHMIRMRQ | 1.758 | 2213 |
| | T121E | LIDIVDQLKNYVNDLVPEFLPAPEDVETNCEWSAFSCFQKA | | |
| | | QLKSANTGNNERIINVSIKKLKRKPPSDNAGGRQKHRLECP | | |
| | | SCDSYEKKPPKEFLERFKSLLQKMIHQHLSSRTHGSEDS | | |
| IL-21_5794 | A112P, R115D | MRSSPGNMERIVICLMVIFLGTLVHKSSSQGQDRHMIRMRQ | 1.758 | 2214 |
| | | LIDIVDQLKNYVNDLVPEFLPAPEDVETNCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_5348 | N70G, A112P, | MRSSPGNMERIVICLMVIFLGTLVHKSSSQGQDRHMIRMRQ | 1.762 | 2215 |
| | R114Q, T121E | LIDIVDQLKNYVNDLVPEFLPAPEDVETGCEWSAFSCFQKA | | |
| | | QLKSANTGNNERIINVSIKKLKRKPPSTNPGQRQKHRLECP | | |
| | | SCDSYEKKPPKEFLERFKSLLQKMIHQHLSSRTHGSEDS | | |
| IL-21_6061 | H35F, I45T, | MRSSPGNMERIVICLMVIFLGTLVHKSSSQGQDREMIRMRQ | 1.771 | 2216 |
| | A112P, R114Q | LIDTVDQLKNYVNDLVPEFLPAPEDVETNCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5773 | H118D, T121E | MRSSPGNMERIVICLMVIFLGTLVHKSSSQGQDRHMIRMRQ | 1.772 | 2217 |
| | | LIDIVDQLKNYVNDLVPEFLPAPEDVETNCEWSAFSCFQKA | | |
| | | QLKSANTGNNERIINVSIKKLKRKPPSTNAGRRQKDRLECP | | |
| | | SCDSYEKKPPKEFLERFKSLLQKMIHQHLSSRTHGSEDS | | |
| IL-21_5838 | N70G, R115G | MRSSPGNMERIVICLMVIFLGTLVHKSSSQGQDRHMIRMRQ | 1.772 | 2218 |
| | | LIDIVDQLKNYVNDLVPEFLPAPEDVETGCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_6301 | H35F, P107V, | MRSSPGNMERIVICLMVIFLGTLVHKSSSQGQDREMIRMRQ | 1.773 | 2219 |
| | R114Q | LIDIVDQLKNYVNDLVPEFLPAPEDVETNCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5747 | N70G, P107V, | MRSSPGNMERIVICLMVIFLGTLVHKSSSQGQDRHMIRMRQ | 1.774 | 2220 |
| | R114G | LIDIVDQLKNYVNDLVPEFLPAPEDVETGCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_6392 | I45T, R115D | MRSSPGNMERIVICLMVIFLGTLVHKSSSQGQDRHMIRMRQ | 1.776 | 2221 |
| | | LIDTVDQLKNYVNDLVPEFLPAPEDVETNCEWSAFSCFQKA | | |
| | | QLKSANTGNNERIINVSIKKLKRKPPSTNAGRDQKHRLTCP | | |
| | | SCDSYEKKPPKEFLERFKSLLQKMIHQHLSSRTHGSEDS | | |
| IL-21_5593 | P107V, R114Q, | MRSSPGNMERIVICLMVIFLGTLVHKSSSQGQDRHMIRMRQ | 1.777 | 2222 |
| | T121E | LIDIVDQLKNYVNDLVPEFLPAPEDVETNCEWSAFSCFQKA | | |
| | | QLKSANTGNNERIINVSIKKLKRKVPSTNAGQRQKHRLECP | | |
| | | SCDSYEKKPPKEFLERFKSLLQKMIHQHLSSRTHGSEDS | | |
| IL-21_5403 | N70G, P107V, | MRSSPGNMERIVICLMVIFLGTLVHKSSSQGQDRHMIRMRQ | 1.779 | 2223 |
| | A112P, R114G | LIDIVDQLKNYVNDLVPEFLPAPEDVETGCEWSAFSCFQKA | | |
| | | QLKSANTGNNERIINVSIKKLKRKVPSTNPGGRQKHRLTCP | | |
| | | SCDSYEKKPPKEFLERFKSLLQKMIHQHLSSRTHGSEDS | | |
| IL-21_4978 | T110D, A112P, | MRSSPGNMERIVICLMVIFLGTLVHKSSSQGQDRHMIRMRQ | 1.779 | 2224 |
| | R114G, T121E | LIDIVDQLKNYVNDLVPEFLPAPEDVETNCEWSAFSCFQKA | | |
| | | QLKSANTGNNERIINVSIKKLKRKPPSDNPGGRQKHRLECP | | |
| | | SCDSYEKKPPKEFLERFKSLLQKMIHQHLSSRTHGSEDS | | |
| IL-21_6303 | H35F, P107V, | MRSSPGNMERIVICLMVIFLGTLVHKSSSQGQDREMIRMRQ | 1.783 | 2225 |
| | T110D | LIDIVDQLKNYVNDLVPEFLPAPEDVETNCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5013 | P107V, A112P, | MRSSPGNMERIVICLMVIFLGTLVHKSSSQGQDRHMIRMRQ | 1.786 | 2226 |
| | R119T, T121E | LIDIVDQLKNYVNDLVPEFLPAPEDVETNCEWSAFSCFQKA | | |
| | | QLKSANTGNNERIINVSIKKLKRKVPSTNPGRRQKHTLECP | | |
| | | SCDSYEKKPPKEFLERFKSLLQKMIHQHLSSRTHGSEDS | | |
| IL-21_6345 | H35I, A112P, | MRSSPGNMERIVICLMVIFLGTLVHKSSSQGQDRIMIRMRQ | 1.787 | 2227 |
| | R114Q | LIDIVDQLKNYVNDLVPEFLPAPEDVETNCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5399 | N70G, P107V, | MRSSPGNMERIVICLMVIFLGTLVHKSSSQGQDRHMIRMRQ | 1.79 | 2228 |
| | A112P, R119T | LIDIVDQLKNYVNDLVPEFLPAPEDVETGCEWSAFSCFQKA | | |
| | | QLKSANTGNNERIINVSIKKLKRKVPSTNPGRRQKHTLTCP | | |
| | | SCDSYEKKPPKEFLERFKSLLQKMIHQHLSSRTHGSEDS | | |
| IL-21_5404 | N70G, P107V, | MRSSPGNMERIVICLMVIFLGTLVHKSSSQGQDRHMIRMRQ | 1.792 | 2229 |
| | A112P, R114Q | LIDIVDQLKNYVNDLVPEFLPAPEDVETGCEWSAFSCFQKA | | |
| | | QLKSANTGNNERIINVSIKKLKRKVPSTNPGQRQKHRLTCP | | |
| | | SCDSYEKKPPKEFLERFKSLLQKMIHQHLSSRTHGSEDS | | |
| IL-21_5516 | R114G, R119T, | MRSSPGNMERIVICLMVIFLGTLVHKSSSQGQDRHMIRMRQ | 1.792 | 2230 |
| | T121E | LIDIVDQLKNYVNDLVPEFLPAPEDVETNCEWSAFSCFQKA | | |
| | | QLKSANTGNNERIINVSIKKLKRKPPSTNAGGRQKHTLECP | | |
| | | SCDSYEKKPPKEFLERFKSLLQKMIHQHLSSRTHGSEDS | | |
| IL-21_5556 | T110D, R115G, | MRSSPGNMERIVICLMVIFLGTLVHKSSSQGQDRHMIRMRQ | 1.793 | 2231 |
| | T121E | LIDIVDQLKNYVNDLVPEFLPAPEDVETNCEWSAFSCFQKA | | |
| | | QLKSANTGNNERIINVSIKKLKRKPPSDNAGRGQKHRLECP | | |
| | | SCDSYEKKPPKEFLERFKSLLQKMIHQHLSSRTHGSEDS | | |
| IL-21_5051 | P107V, T110D, | MRSSPGNMERIVICLMVIFLGTLVHKSSSQGQDRHMIRMRQ | 1.795 | 2232 |
| | A112P, T121E | LIDIVDQLKNYVNDLVPEFLPAPEDVETNCEWSAFSCFQKA | | |
| | | QLKSANTGNNERIINVSIKKLKRKVPSDNPGRRQKHRLECP | | |
| | | SCDSYEKKPPKEFLERFKSLLQKMIHQHLSSRTHGSEDS | | |
| IL-21_5536 | A112P, H118D, | MRSSPGNMERIVICLMVIFLGTLVHKSSSQGQDRHMIRMRQ | 1.798 | 2233 |
| | R119T | LIDIVDQLKNYVNDLVPEFLPAPEDVETNCEWSAFSCFQKA | | |
| | | QLKSANTGNNERIINVSIKKLKRKPPSTNPGRRQKDTLTCP | | |
| | | SCDSYEKKPPKEFLERFKSLLQKMIHQHLSSRTHGSEDS | | |
| IL-21_6330 | H35F, I45T, | MRSSPGNMERIVICLMVIFLGTLVHKSSSQGQDRFMIRMRQ | 1.803 | 2234 |
| | R114G | LIDTVDQLKNYVNDLVPEFLPAPEDVETNCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_6384 | H351, I45T, | MRSSPGNMERIVICLMVIFLGTLVHKSSSQGQDRIMIRMRQ | 1.804 | 2235 |
| | R114Q | LIDTVDQLKNYVNDLVPEFLPAPEDVETNCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5807 | P107V, H118D | MRSSPGNMERIVICLMVIFLGTLVHKSSSQGQDRHMIRMRQ | 1.808 | 2236 |
| | | LIDIVDQLKNYVNDLVPEFLPAPEDVETNCEWSAFSCFQKA | | |
| | | QLKSANTGNNERIINVSIKKLKRKVPSTNAGRRQKDRLTCP | | |
| | | SCDSYEKKPPKEFLERFKSLLQKMIHQHLSSRTHGSEDS | | |
| IL-21_5575 | T110D, A112P, | MRSSPGNMERIVICLMVIFLGTLVHKSSSQGQDRHMIRMRQ | 1.809 | 2237 |
| | R115G | LIDIVDQLKNYVNDLVPEFLPAPEDVETNCEWSAFSCFQKA | | |
| | | QLKSANTGNNERIINVSIKKLKRKPPSDNPGRGQKHRLTCP | | |
| | | SCDSYEKKPPKEFLERFKSLLQKMIHQHLSSRTHGSEDS | | |
| IL-21_2896 | R115D, T121E | MRSSPGNMERIVICLMVIFLGTLVHKSSSQGQDRHMIRMRQ | 1.81 | 2238 |
| | | LIDIVDQLKNYVNDLVPEFLPAPEDVETNCEWSAFSCFQKA | | |
| | | QLKSANTGNNERIINVSIKKLKRKPPSTNAGRDQKHRLECP | | |
| | | SCDSYEKKPPKEFLERFKSLLQKMIHQHLSSRTHGSEDS | | |
| IL-21_6072 | H35F, I45T, | MRSSPGNMERIVICLMVIFLGTLVHKSSSQGQDREMIRMRQ | 1.81 | 2239 |
| | P107V, T110D | LIDTVDQLKNYVNDLVPEFLPAPEDVETNCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5334 | N70G, A112P, | MRSSPGNMERIVICLMVIFLGTLVHKSSSQGQDRHMIRMRQ | 1.814 | 2240 |
| | R119T, T121E | LIDIVDQLKNYVNDLVPEFLPAPEDVETGCEWSAFSCFQKA | | |
| | | QLKSANTGNNERIINVSIKKLKRKPPSTNPGRRQKHTLECP | | |
| | | SCDSYEKKPPKEFLERFKSLLQKMIHQHLSSRTHGSEDS | | |
| IL-21_6297 | H35F, T110D, | MRSSPGNMERIVICLMVIFLGTLVHKSSSQGQDREMIRMRQ | 1.817 | 2241 |
| | A112P | LIDIVDQLKNYVNDLVPEFLPAPEDVETNCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5604 | P107V, T110D, | MRSSPGNMERIVICLMVIFLGTLVHKSSSQGQDRHMIRMRQ | 1.817 | 2242 |
| | T121E | LIDIVDQLKNYVNDLVPEFLPAPEDVETNCEWSAFSCFQKA | | |
| | | QLKSANTGNNERIINVSIKKLKRKVPSDNAGRRQKHRLECP | | |
| | | SCDSYEKKPPKEFLERFKSLLQKMIHQHLSSRTHGSEDS | | |
| IL-21_5519 | R114G, R115G, | MRSSPGNMERIVICLMVIFLGTLVHKSSSQGQDRHMIRMRQ | 1.817 | 2243 |
| | T121E | LIDIVDQLKNYVNDLVPEFLPAPEDVETNCEWSAFSCFQKA | | |
| | | QLKSANTGNNERIINVSIKKLKRKPPSTNAGGGQKHRLECP | | |
| | | SCDSYEKKPPKEFLERFKSLLQKMIHQHLSSRTHGSEDS | | |
| IL-21_6266 | I45T, V98E, | MRSSPGNMERIVICLMVIFLGTLVHKSSSQGQDRHMIRMRQ | 1.819 | 2244 |
| | A112P | LIDTVDQLKNYVNDLVPEFLPAPEDVETNCEWSAFSCFQKA | | |
| | | QLKSANTGNNERIINESIKKLKRKPPSTNPGRRQKHRLTCP | | |
| | | SCDSYEKKPPKEFLERFKSLLQKMIHQHLSSRTHGSEDS | | |
| IL-21_5808 | P107V, R115G | MRSSPGNMERIVICLMVIFLGTLVHKSSSQGQDRHMIRMRQ | 1.819 | 2245 |
| | | LIDIVDQLKNYVNDLVPEFLPAPEDVETNCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5743 | N70G, P107V, | MRSSPGNMERIVICLMVIFLGTLVHKSSSQGQDRHMIRMRQ | 1.822 | 2246 |
| | R119T | LIDIVDQLKNYVNDLVPEFLPAPEDVETGCEWSAFSCFQKA | | |
| | | QLKSANTGNNERIINVSIKKLKRKVPSTNAGRRQKHTLTCP | | |
| | | SCDSYEKKPPKEFLERFKSLLQKMIHQHLSSRTHGSEDS | | |
| IL-21_5538 | A112P, R115G, | MRSSPGNMERIVICLMVIFLGTLVHKSSSQGQDRHMIRMRQ | 1.824 | 2247 |
| | R119T | LIDIVDQLKNYVNDLVPEFLPAPEDVETNCEWSAFSCFQKA | | |
| | | QLKSANTGNNERIINVSIKKLKRKPPSTNPGRGQKHTLTCP | | |
| | | SCDSYEKKPPKEFLERFKSLLQKMIHQHLSSRTHGSEDS | | |
| IL-21_5022 | P107V, A112P, | MRSSPGNMERIVICLMVIFLGTLVHKSSSQGQDRHMIRMRQ | 1.825 | 2248 |
| | R114G, T121E | LIDIVDQLKNYVNDLVPEFLPAPEDVETNCEWSAFSCFQKA | | |
| | | QLKSANTGNNERIINVSIKKLKRKVPSTNPGGRQKHRLECP | | |
| | | SCDSYEKKPPKEFLERFKSLLQKMIHQHLSSRTHGSEDS | | |
| IL-21_6323 | H35F, N70G, | MRSSPGNMERIVICLMVIFLGTLVHKSSSQGQDREMIRMRQ | 1.826 | 2249 |
| | A112P | LIDIVDQLKNYVNDLVPEFLPAPEDVETGCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5577 | T110D, A112P, | MRSSPGNMERIVICLMVIFLGTLVHKSSSQGQDRHMIRMRQ | 1.826 | 2250 |
| | R114G | LIDIVDQLKNYVNDLVPEFLPAPEDVETNCEWSAFSCFQKA | | |
| | | QLKSANTGNNERIINVSIKKLKRKPPSDNPGGRQKHRLTCP | | |
| | | SCDSYEKKPPKEFLERFKSLLQKMIHQHLSSRTHGSEDS | | |
| IL-21_4983 | T110D, A112P, | MRSSPGNMERIVICLMVIFLGTLVHKSSSQGQDRHMIRMRQ | 1.829 | 2251 |
| | R114Q, T121E | LIDIVDQLKNYVNDLVPEFLPAPEDVETNCEWSAFSCFQKA | | |
| | | QLKSANTGNNERIINVSIKKLKRKPPSDNPGQRQKHRLECP | | |
| | | SCDSYEKKPPKEFLERFKSLLQKMIHQHLSSRTHGSEDS | | |
| IL-21_6207 | H35I, I45T, | MRSSPGNMERIVICLMVIFLGTLVHKSSSQGQDRIMIRMRQ | 1.83 | 2252 |
| | A112P, R114Q | LIDTVDQLKNYVNDLVPEFLPAPEDVETNCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_6093 | H35F, I45T, | MRSSPGNMERIVICLMVIFLGTLVHKSSSQGQDREMIRMRQ | 1.83 | 2253 |
| | N70G, T110D | LIDTVDQLKNYVNDLVPEFLPAPEDVETGCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_6204 | H35I, I45T, | MRSSPGNMERIVICLMVIFLGTLVHKSSSQGQDRIMIRMRQ | 1.836 | 2254 |
| | A112P, R115G | LIDTVDQLKNYVNDLVPEFLPAPEDVETNCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5974 | H35F, P107V, | MRSSPGNMERIVICLMVIFLGTLVHKSSSQGQDRFMIRMRQ | 1.837 | 2255 |
| | T110D, A112P | LIDIVDQLKNYVNDLVPEFLPAPEDVETNCEWSAFSCFQKA | | |
| | | QLKSANTGNNERIINVSIKKLKRKVPSDNPGRRQKHRLTCP | | |
| | | SCDSYEKKPPKEFLERFKSLLQKMIHQHLSSRTHGSEDS | | |
| IL-21_2726 | N70G, R114G | MRSSPGNMERIVICLMVIFLGTLVHKSSSQGQDRHMIRMRQ | 1.837 | 2256 |
| | | LIDIVDQLKNYVNDLVPEFLPAPEDVETGCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_6067 | H35F, I45T, | MRSSPGNMERIVICLMVIFLGTLVHKSSSQGQDRFMIRMRQ | 1.839 | 2257 |
| | P107V, R115G | LIDTVDQLKNYVNDLVPEFLPAPEDVETNCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5742 | N70G, P107V, | MRSSPGNMERIVICLMVIFLGTLVHKSSSQGQDRHMIRMRQ | 1.842 | 2258 |
| | T121E | LIDIVDQLKNYVNDLVPEFLPAPEDVETGCEWSAFSCFQKA | | |
| | | QLKSANTGNNERIINVSIKKLKRKVPSTNAGRRQKHRLECP | | |
| | | SCDSYEKKPPKEFLERFKSLLQKMIHQHLSSRTHGSEDS | | |
| IL-21_6374 | H35I, N70G, | MRSSPGNMERIVICLMVIFLGTLVHKSSSQGQDRIMIRMRQ | 1.853 | 2259 |
| | R114G | LIDIVDQLKNYVNDLVPEFLPAPEDVETGCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5803 | T110D, R114Q | MRSSPGNMERIVICLMVIFLGTLVHKSSSQGQDRHMIRMRQ | 1.855 | 2260 |
| | | LIDIVDQLKNYVNDLVPEFLPAPEDVETNCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5837 | N70G, H118D | MRSSPGNMERIVICLMVIFLGTLVHKSSSQGQDRHMIRMRQ | 1.856 | 2261 |
| | | LIDIVDQLKNYVNDLVPEFLPAPEDVETGCEWSAFSCFQKA | | |
| | | QLKSANTGNNERIINVSIKKLKRKPPSTNAGRRQKDRLTCP | | |
| | | SCDSYEKKPPKEFLERFKSLLQKMIHQHLSSRTHGSEDS | | |
| IL-21_5787 | R114Q, H118D | MRSSPGNMERIVICLMVIFLGTLVHKSSSQGQDRHMIRMRQ | 1.858 | 2262 |
| | | LIDIVDQLKNYVNDLVPEFLPAPEDVETNCEWSAFSCFQKA | | |
| | | QLKSANTGNNERIINVSIKKLKRKPPSTNAGQRQKDRLTCP | | |
| | | SCDSYEKKPPKEFLERFKSLLQKMIHQHLSSRTHGSEDS | | |
| IL-21_6058 | H35F, I45T, | MRSSPGNMERIVICLMVIFLGTLVHKSSSQGQDREMIRMRQ | 1.861 | 2263 |
| | A112P, R115G | LIDTVDQLKNYVNDLVPEFLPAPEDVETNCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5922 | I45T, N70G, | MRSSPGNMERIVICLMVIFLGTLVHKSSSQGQDRHMIRMRQ | 1.866 | 2264 |
| | A112P, R114G | LIDTVDQLKNYVNDLVPEFLPAPEDVETGCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_6060 | H35F, I45T, | MRSSPGNMERIVICLMVIFLGTLVHKSSSQGQDREMIRMRQ | 1.866 | 2265 |
| | A112P, R114G | LIDTVDQLKNYVNDLVPEFLPAPEDVETNCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_6300 | H35F, P107V, | MRSSPGNMERIVICLMVIFLGTLVHKSSSQGQDRFMIRMRQ | 1.867 | 2266 |
| | R114G | LIDIVDQLKNYVNDLVPEFLPAPEDVETNCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5343 | N70G, A112P, | MRSSPGNMERIVICLMVIFLGTLVHKSSSQGQDRHMIRMRQ | 1.869 | 2267 |
| | R114G, T121E | LIDIVDQLKNYVNDLVPEFLPAPEDVETGCEWSAFSCFQKA | | |
| | | QLKSANTGNNERIINVSIKKLKRKPPSTNPGGRQKHRLECP | | |
| | | SCDSYEKKPPKEFLERFKSLLQKMIHQHLSSRTHGSEDS | | |
| IL-21_5027 | P107V, A112P, | MRSSPGNMERIVICLMVIFLGTLVHKSSSQGQDRHMIRMRQ | 1.87 | 2268 |
| | R114Q, T121E | LIDIVDQLKNYVNDLVPEFLPAPEDVETNCEWSAFSCFQKA | | |
| | | QLKSANTGNNERIINVSIKKLKRKVPSTNPGQRQKHRLECP | | |
| | | SCDSYEKKPPKEFLERFKSLLQKMIHQHLSSRTHGSEDS | | |
| IL-21_5923 | I45T, N70G, | MRSSPGNMERIVICLMVIFLGTLVHKSSSQGQDRHMIRMRQ | 1.875 | 2269 |
| | A112P, R114Q | LIDTVDQLKNYVNDLVPEFLPAPEDVETGCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5549 | A112P, R114Q, | MRSSPGNMERIVICLMVIFLGTLVHKSSSQGQDRHMIRMRQ | 1.876 | 2270 |
| | R119T | LIDIVDQLKNYVNDLVPEFLPAPEDVETNCEWSAFSCFQKA | | |
| | | QLKSANTGNNERIINVSIKKLKRKPPSTNPGQRQKHTLTCP | | |
| | | SCDSYEKKPPKEFLERFKSLLQKMIHQHLSSRTHGSEDS | | |
| IL-21_5799 | T110D, H118D | MRSSPGNMERIVICLMVIFLGTLVHKSSSQGQDRHMIRMRQ | 1.88 | 2271 |
| | | LIDIVDQLKNYVNDLVPEFLPAPEDVETNCEWSAFSCFQKA | | |
| | | QLKSANTGNNERIINVSIKKLKRKPPSDNAGRRQKDRLTCP | | |
| | | SCDSYEKKPPKEFLERFKSLLQKMIHQHLSSRTHGSEDS | | |
| IL-21_5813 | P107V, T110D | MRSSPGNMERIVICLMVIFLGTLVHKSSSQGQDRHMIRMRQ | 1.881 | 2272 |
| | | LIDIVDQLKNYVNDLVPEFLPAPEDVETNCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_6183 | H35I, N70G, | MRSSPGNMERIVICLMVIFLGTLVHKSSSQGQDRIMIRMRQ | 1.881 | 2273 |
| | P107V, A112P | LIDIVDQLKNYVNDLVPEFLPAPEDVETGCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_5733 | N70G, A112P, | MRSSPGNMERIVICLMVIFLGTLVHKSSSQGQDRHMIRMRQ | 1.885 | 2274 |
| | R114Q | LIDIVDQLKNYVNDLVPEFLPAPEDVETGCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_6213 | H35I, I45T, | MRSSPGNMERIVICLMVIFLGTLVHKSSSQGQDRIMIRMRQ | 1.885 | 2275 |
| | P107V, R115G | LIDTVDQLKNYVNDLVPEFLPAPEDVETNCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5372 | N70G, T110D, | MRSSPGNMERIVICLMVIFLGTLVHKSSSQGQDRHMIRMRQ | 1.887 | 2276 |
| | A112P, T121E | LIDIVDQLKNYVNDLVPEFLPAPEDVETGCEWSAFSCFQKA | | |
| | | QLKSANTGNNERIINVSIKKLKRKPPSDNPGRRQKHRLECP | | |
| | | SCDSYEKKPPKEFLERFKSLLQKMIHQHLSSRTHGSEDS | | |
| IL-21_6218 | H35I, I45T, | MRSSPGNMERIVICLMVIFLGTLVHKSSSQGQDRIMIRMRQ | 1.888 | 2277 |
| | P107V, T110D | LIDTVDQLKNYVNDLVPEFLPAPEDVETNCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5730 | N70G, A112P, | MRSSPGNMERIVICLMVIFLGTLVHKSSSQGQDRHMIRMRQ | 1.889 | 2278 |
| | R115G | LIDIVDQLKNYVNDLVPEFLPAPEDVETGCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5744 | N70G, P107V, | MRSSPGNMERIVICLMVIFLGTLVHKSSSQGQDRHMIRMRQ | 1.892 | 2279 |
| | H118D | LIDIVDQLKNYVNDLVPEFLPAPEDVETGCEWSAFSCFQKA | | |
| | | QLKSANTGNNERIINVSIKKLKRKVPSTNAGRRQKDRLTCP | | |
| | | SCDSYEKKPPKEFLERFKSLLQKMIHQHLSSRTHGSEDS | | |
| IL-21_6243 | I45T, R114G, | MRSSPGNMERIVICLMVIFLGTLVHKSSSQGQDRHMIRMRQ | 1.893 | 2280 |
| | R115G | LIDTVDQLKNYVNDLVPEFLPAPEDVETNCEWSAFSCFQKA | | |
| | | QLKSANTGNNERIINVSIKKLKRKPPSTNAGGGQKHRLTCP | | |
| | | SCDSYEKKPPKEFLERFKSLLQKMIHQHLSSRTHGSEDS | | |
| IL-21_5810 | P107V, R114G | MRSSPGNMERIVICLMVIFLGTLVHKSSSQGQDRHMIRMRQ | 1.898 | 2281 |
| | | LIDIVDQLKNYVNDLVPEFLPAPEDVETNCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_5729 | N70G, A112P, | MRSSPGNMERIVICLMVIFLGTLVHKSSSQGQDRHMIRMRQ | 1.91 | 2282 |
| | H118D | LIDIVDQLKNYVNDLVPEFLPAPEDVETGCEWSAFSCFQKA | | |
| | | QLKSANTGNNERIINVSIKKLKRKPPSTNPGRRQKDRLTCP | | |
| | | SCDSYEKKPPKEFLERFKSLLQKMIHQHLSSRTHGSEDS | | |
| IL-21_6383 | H35I, I45T, | MRSSPGNMERIVICLMVIFLGTLVHKSSSQGQDRIMIRMRQ | 1.912 | 2283 |
| | R114G | LIDTVDQLKNYVNDLVPEFLPAPEDVETNCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_6215 | H35I, I45T, | MRSSPGNMERIVICLMVIFLGTLVHKSSSQGQDRIMIRMRQ | 1.914 | 2284 |
| | P107V, R114G | LIDTVDQLKNYVNDLVPEFLPAPEDVETNCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_6278 | I45T, N70G, | MRSSPGNMERIVICLMVIFLGTLVHKSSSQGQDRHMIRMRQ | 1.916 | 2285 |
| | R114G | LIDTVDQLKNYVNDLVPEFLPAPEDVETGCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_5550 | A112P, R114Q, | MRSSPGNMERIVICLMVIFLGTLVHKSSSQGQDRHMIRMRQ | 1.916 | 2286 |
| | H118D | LIDIVDQLKNYVNDLVPEFLPAPEDVETNCEWSAFSCFQKA | | |
| | | QLKSANTGNNERIINVSIKKLKRKPPSTNPGQRQKDRLTCP | | |
| | | SCDSYEKKPPKEFLERFKSLLQKMIHQHLSSRTHGSEDS | | |
| IL-21_6415 | H35I, R114Q | MRSSPGNMERIVICLMVIFLGTLVHKSSSQGQDRIMIRMRQ | 1.917 | 2287 |
| | | LIDIVDQLKNYVNDLVPEFLPAPEDVETNCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5785 | R114Q, T121E | MRSSPGNMERIVICLMVIFLGTLVHKSSSQGQDRHMIRMRQ | 1.917 | 2288 |
| | | LIDIVDQLKNYVNDLVPEFLPAPEDVETNCEWSAFSCFQKA | | |
| | | QLKSANTGNNERIINVSIKKLKRKPPSTNAGQRQKHRLECP | | |
| | | SCDSYEKKPPKEFLERFKSLLQKMIHQHLSSRTHGSEDS | | |
| IL-21_5578 | T110D, A112P, | MRSSPGNMERIVICLMVIFLGTLVHKSSSQGQDRHMIRMRQ | 1.918 | 2289 |
| | R114Q | LIDIVDQLKNYVNDLVPEFLPAPEDVETNCEWSAFSCFQKA | | |
| | | QLKSANTGNNERIINVSIKKLKRKPPSDNPGQRQKHRLTCP | | |
| | | SCDSYEKKPPKEFLERFKSLLQKMIHQHLSSRTHGSEDS | | |
| IL-21_6256 | I45T, P107V, | MRSSPGNMERIVICLMVIFLGTLVHKSSSQGQDRHMIRMRQ | 1.921 | 2290 |
| | R115G | LIDTVDQLKNYVNDLVPEFLPAPEDVETNCEWSAFSCFQKA | | |
| | | QLKSANTGNNERIINVSIKKLKRKVPSTNAGRGQKHRLTCP | | |
| | | SCDSYEKKPPKEFLERFKSLLQKMIHQHLSSRTHGSEDS | | |
| IL-21_5870 | I45T, P107V, | MRSSPGNMERIVICLMVIFLGTLVHKSSSQGQDRHMIRMRQ | 1.923 | 2291 |
| | T110D, A112P | LIDTVDQLKNYVNDLVPEFLPAPEDVETNCEWSAFSCFQKA | | |
| | | QLKSANTGNNERIINVSIKKLKRKVPSDNPGRRQKHRLTCP | | |
| | | SCDSYEKKPPKEFLERFKSLLQKMIHQHLSSRTHGSEDS | | |
| IL-21_5551 | A112P, R114Q, | MRSSPGNMERIVICLMVIFLGTLVHKSSSQGQDRHMIRMRQ | 1.925 | 2292 |
| | R115G | LIDIVDQLKNYVNDLVPEFLPAPEDVETNCEWSAFSCFQKA | | |
| | | QLKSANTGNNERIINVSIKKLKRKPPSTNPGQGQKHRLTCP | | |
| | | SCDSYEKKPPKEFLERFKSLLQKMIHQHLSSRTHGSEDS | | |
| IL-21_5599 | P107V, A112P, | MRSSPGNMERIVICLMVIFLGTLVHKSSSQGQDRHMIRMRQ | 1.926 | 2293 |
| | H118D | LIDIVDQLKNYVNDLVPEFLPAPEDVETNCEWSAFSCFQKA | | |
| | | QLKSANTGNNERIINVSIKKLKRKVPSTNPGRRQKDRLTCP | | |
| | | SCDSYEKKPPKEFLERFKSLLQKMIHQHLSSRTHGSEDS | | |
| IL-21_5865 | I45T, P107V, | MRSSPGNMERIVICLMVIFLGTLVHKSSSQGQDRHMIRMRQ | 1.927 | 2294 |
| | A112P, R114Q | LIDTVDQLKNYVNDLVPEFLPAPEDVETNCEWSAFSCFQKA | | |
| | | QLKSANTGNNERIINVSIKKLKRKVPSTNPGQRQKHRLTCP | | |
| | | SCDSYEKKPPKEFLERFKSLLQKMIHQHLSSRTHGSEDS | | |
| IL-21_6212 | H351, I45T, | MRSSPGNMERIVICLMVIFLGTLVHKSSSQGQDRIMIRMRQ | 1.934 | 2295 |
| | T110D, A112P | LIDTVDQLKNYVNDLVPEFLPAPEDVETNCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5412 | N70G, P107V, | MRSSPGNMERIVICLMVIFLGTLVHKSSSQGQDRHMIRMRQ | 1.935 | 2296 |
| | T110D, A112P | LIDIVDQLKNYVNDLVPEFLPAPEDVETGCEWSAFSCFQKA | | |
| | | QLKSANTGNNERIINVSIKKLKRKVPSDNPGRRQKHRLTCP | | |
| | | SCDSYEKKPPKEFLERFKSLLQKMIHQHLSSRTHGSEDS | | |
| IL-21_5600 | P107V, A112P, | MRSSPGNMERIVICLMVIFLGTLVHKSSSQGQDRHMIRMRQ | 1.936 | 2297 |
| | R115G | LIDIVDQLKNYVNDLVPEFLPAPEDVETNCEWSAFSCFQKA | | |
| | | QLKSANTGNNERIINVSIKKLKRKVPSTNPGRGQKHRLTCP | | |
| | | SCDSYEKKPPKEFLERFKSLLQKMIHQHLSSRTHGSEDS | | |
| IL-21_4969 | T110D, A112P, | MRSSPGNMERIVICLMVIFLGTLVHKSSSQGQDRHMIRMRQ | 1.947 | 2298 |
| | R119T, T121E | LIDIVDQLKNYVNDLVPEFLPAPEDVETNCEWSAFSCFQKA | | |
| | | QLKSANTGNNERIINVSIKKLKRKPPSDNPGRRQKHTLECP | | |
| | | SCDSYEKKPPKEFLERFKSLLQKMIHQHLSSRTHGSEDS | | |
| IL-21_5862 | I45T, P107V, | MRSSPGNMERIVICLMVIFLGTLVHKSSSQGQDRHMIRMRQ | 1.952 | 2299 |
| | A112P, R115G | LIDTVDQLKNYVNDLVPEFLPAPEDVETNCEWSAFSCFQKA | | |
| | | QLKSANTGNNERIINVSIKKLKRKVPSTNPGRGQKHRLTCP | | |
| | | SCDSYEKKPPKEFLERFKSLLQKMIHQHLSSRTHGSEDS | | |
| IL-21_6248 | I45T, A112P, | MRSSPGNMERIVICLMVIFLGTLVHKSSSQGQDRHMIRMRQ | 1.953 | 2300 |
| | R115D | LIDTVDQLKNYVNDLVPEFLPAPEDVETNCEWSAFSCFQKA | | |
| | | QLKSANTGNNERIINVSIKKLKRKPPSTNPGRDQKHRLTCP | | |
| | | SCDSYEKKPPKEFLERFKSLLQKMIHQHLSSRTHGSEDS | | |
| IL-21_6350 | H35I, T110D, | MRSSPGNMERIVICLMVIFLGTLVHKSSSQGQDRIMIRMRQ | 1.957 | 2301 |
| | A112P | LIDIVDQLKNYVNDLVPEFLPAPEDVETNCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5573 | T110D, A112P, | MRSSPGNMERIVICLMVIFLGTLVHKSSSQGQDRHMIRMRQ | 1.96 | 2302 |
| | R119T | LIDIVDQLKNYVNDLVPEFLPAPEDVETNCEWSAFSCFQKA | | |
| | | QLKSANTGNNERIINVSIKKLKRKPPSDNPGRRQKHTLTCP | | |
| | | SCDSYEKKPPKEFLERFKSLLQKMIHQHLSSRTHGSEDS | | |
| IL-21_6398 | I45T, V98E | MRSSPGNMERIVICLMVIFLGTLVHKSSSQGQDRHMIRMRQ | 1.96 | 2303 |
| | | LIDTVDQLKNYVNDLVPEFLPAPEDVETNCEWSAFSCFQKA | | |
| | | QLKSANTGNNERIINESIKKLKRKPPSTNAGRRQKHRLTCP | | |
| | | SCDSYEKKPPKEFLERFKSLLQKMIHQHLSSRTHGSEDS | | |
| IL-21_5537 | A112P, R115G, | MRSSPGNMERIVICLMVIFLGTLVHKSSSQGQDRHMIRMRQ | 1.962 | 2304 |
| | T121E | LIDIVDQLKNYVNDLVPEFLPAPEDVETNCEWSAFSCFQKA | | |
| | | QLKSANTGNNERIINVSIKKLKRKPPSTNPGRGQKHRLECP | | |
| | | SCDSYEKKPPKEFLERFKSLLQKMIHQHLSSRTHGSEDS | | |
| IL-21_5708 | N70G, R119T, | MRSSPGNMERIVICLMVIFLGTLVHKSSSQGQDRHMIRMRQ | 1.964 | 2305 |
| | T121E | LIDIVDQLKNYVNDLVPEFLPAPEDVETGCEWSAFSCFQKA | | |
| | | QLKSANTGNNERIINVSIKKLKRKPPSTNAGRRQKHTLECP | | |
| | | SCDSYEKKPPKEFLERFKSLLQKMIHQHLSSRTHGSEDS | | |
| IL-21_6386 | H351, I45T, | MRSSPGNMERIVICLMVIFLGTLVHKSSSQGQDRIMIRMRQ | 1.97 | 2306 |
| | T110D | LIDTVDQLKNYVNDLVPEFLPAPEDVETNCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5544 | A112P, R114G, | MRSSPGNMERIVICLMVIFLGTLVHKSSSQGQDRHMIRMRQ | 1.971 | 2307 |
| | R119T | LIDIVDQLKNYVNDLVPEFLPAPEDVETNCEWSAFSCFQKA | | |
| | | QLKSANTGNNERIINVSIKKLKRKPPSTNPGGRQKHTLTCP | | |
| | | SCDSYEKKPPKEFLERFKSLLQKMIHQHLSSRTHGSEDS | | |
| IL-21_6331 | H35F, I45T, | MRSSPGNMERIVICLMVIFLGTLVHKSSSQGQDRFMIRMRQ | 1.971 | 2308 |
| | R114Q | LIDTVDQLKNYVNDLVPEFLPAPEDVETNCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_6410 | H35F, N70G | MRSSPGNMERIVICLMVIFLGTLVHKSSSQGQDRFMIRMRQ | 1.974 | 2309 |
| | | LIDIVDQLKNYVNDLVPEFLPAPEDVETGCEWSAFSCFQKA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6254 | I45T, T110D, | MRSSPGNMERIVICLMVIFLGTLVHKSSSQGQDRHMIRMRQ | 1.982 | 2310 |
| | R114Q | LIDTVDQLKNYVNDLVPEFLPAPEDVETNCEWSAFSCFQKA | | |
| | | QLKSANTGNNERIINVSIKKLKRKPPSDNAGQRQKHRLTCP | | |
| | | SCDSYEKKPPKEFLERFKSLLQKMIHQHLSSRTHGSEDS | | |
| IL-21_5535 | A112P, H118D, | MRSSPGNMERIVICLMVIFLGTLVHKSSSQGQDRHMIRMRQ | 1.992 | 2311 |
| | T121E | LIDIVDQLKNYVNDLVPEFLPAPEDVETNCEWSAFSCFQKA | | |
| | | QLKSANTGNNERIINVSIKKLKRKPPSTNPGRRQKDRLECP | | |
| | | SCDSYEKKPPKEFLERFKSLLQKMIHQHLSSRTHGSEDS | | |
| IL-21_5741 | N70G, T110D, | MRSSPGNMERIVICLMVIFLGTLVHKSSSQGQDRHMIRMRQ | 1.998 | 2312 |
| | A112P | LIDIVDQLKNYVNDLVPEFLPAPEDVETGCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5920 | I45T, N70G, | MRSSPGNMERIVICLMVIFLGTLVHKSSSQGQDRHMIRMRQ | 2 | 2313 |
| | A112P, R115G | LIDTVDQLKNYVNDLVPEFLPAPEDVETGCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5611 | P107V, T110D, | MRSSPGNMERIVICLMVIFLGTLVHKSSSQGQDRHMIRMRQ | 2.006 | 2314 |
| | A112P | LIDIVDQLKNYVNDLVPEFLPAPEDVETNCEWSAFSCFQKA | | |
| | | QLKSANTGNNERIINVSIKKLKRKVPSDNPGRRQKHRLTCP | | |
| | | SCDSYEKKPPKEFLERFKSLLQKMIHQHLSSRTHGSEDS | | |
| IL-21_6344 | H35I, A112P, | MRSSPGNMERIVICLMVIFLGTLVHKSSSQGQDRIMIRMRQ | 2.012 | 2315 |
| | R114G | LIDIVDQLKNYVNDLVPEFLPAPEDVETNCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_6421 | H35I, N70G | MRSSPGNMERIVICLMVIFLGTLVHKSSSQGQDRIMIRMRQ | 2.014 | 2316 |
| | | LIDIVDQLKNYVNDLVPEFLPAPEDVETGCEWSAFSCFQKA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5786 | R114Q, R119T | MRSSPGNMERIVICLMVIFLGTLVHKSSSQGQDRHMIRMRQ | 2.015 | 2317 |
| | | LIDIVDQLKNYVNDLVPEFLPAPEDVETNCEWSAFSCFQKA | | |
| | | QLKSANTGNNERIINVSIKKLKRKPPSTNAGQRQKHTLTCP | | |
| | | SCDSYEKKPPKEFLERFKSLLQKMIHQHLSSRTHGSEDS | | |
| IL-21_6407 | H35F, P107V | MRSSPGNMERIVICLMVIFLGTLVHKSSSQGQDRFMIRMRQ | 2.015 | 2318 |
| | | LIDIVDQLKNYVNDLVPEFLPAPEDVETNCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5774 | H118D, R119T | MRSSPGNMERIVICLMVIFLGTLVHKSSSQGQDRHMIRMRQ | 2.023 | 2319 |
| | | LIDIVDQLKNYVNDLVPEFLPAPEDVETNCEWSAFSCFQKA | | |
| | | QLKSANTGNNERIINVSIKKLKRKPPSTNAGRRQKDTLTCP | | |
| | | SCDSYEKKPPKEFLERFKSLLQKMIHQHLSSRTHGSEDS | | |
| IL-21_6325 | H35F, N70G, | MRSSPGNMERIVICLMVIFLGTLVHKSSSQGQDREMIRMRQ | 2.024 | 2320 |
| | P107V | LIDIVDQLKNYVNDLVPEFLPAPEDVETGCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_6281 | I45T, N70G, | MRSSPGNMERIVICLMVIFLGTLVHKSSSQGQDRHMIRMRQ | 2.028 | 2321 |
| | T110D | LIDTVDQLKNYVNDLVPEFLPAPEDVETGCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5934 | I45T, N70G, | MRSSPGNMERIVICLMVIFLGTLVHKSSSQGQDRHMIRMRQ | 2.033 | 2322 |
| | P107V, T110D | LIDTVDQLKNYVNDLVPEFLPAPEDVETGCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_6279 | I45T, N70G, | MRSSPGNMERIVICLMVIFLGTLVHKSSSQGQDRHMIRMRQ | 2.041 | 2323 |
| | R114Q | LIDTVDQLKNYVNDLVPEFLPAPEDVETGCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_6240 | H351, I45T, | MRSSPGNMERIVICLMVIFLGTLVHKSSSQGQDRIMIRMRQ | 2.063 | 2324 |
| | N70G, P107V | LIDTVDQLKNYVNDLVPEFLPAPEDVETGCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5798 | T110D, R119T | MRSSPGNMERIVICLMVIFLGTLVHKSSSQGQDRHMIRMRQ | 2.063 | 2325 |
| | | LIDIVDQLKNYVNDLVPEFLPAPEDVETNCEWSAFSCFQKA | | |
| | | QLKSANTGNNERIINVSIKKLKRKPPSDNAGRRQKHTLTCP | | |
| | | SCDSYEKKPPKEFLERFKSLLQKMIHQHLSSRTHGSEDS | | |
| IL-21_5811 | P107V, R114Q | MRSSPGNMERIVICLMVIFLGTLVHKSSSQGQDRHMIRMRQ | 2.071 | 2326 |
| | | LIDIVDQLKNYVNDLVPEFLPAPEDVETNCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5603 | P107V, A112P, | MRSSPGNMERIVICLMVIFLGTLVHKSSSQGQDRHMIRMRQ | 2.072 | 2327 |
| | R114Q | LIDIVDQLKNYVNDLVPEFLPAPEDVETNCEWSAFSCFQKA | | |
| | | QLKSANTGNNERIINVSIKKLKRKVPSTNPGQRQKHRLTCP | | |
| | | SCDSYEKKPPKEFLERFKSLLQKMIHQHLSSRTHGSEDS | | |
| IL-21_5775 | R115G, T121E | MRSSPGNMERIVICLMVIFLGTLVHKSSSQGQDRHMIRMRQ | 2.073 | 2328 |
| | | LIDIVDQLKNYVNDLVPEFLPAPEDVETNCEWSAFSCFQKA | | |
| | | QLKSANTGNNERIINVSIKKLKRKPPSTNAGRGQKHRLECP | | |
| | | SCDSYEKKPPKEFLERFKSLLQKMIHQHLSSRTHGSEDS | | |
| IL-21_6376 | H35I, N70G, | MRSSPGNMERIVICLMVIFLGTLVHKSSSQGQDRIMIRMRQ | 2.074 | 2329 |
| | A112P | LIDIVDQLKNYVNDLVPEFLPAPEDVETGCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5749 | N70G, P107V, | MRSSPGNMERIVICLMVIFLGTLVHKSSSQGQDRHMIRMRQ | 2.078 | 2330 |
| | A112P | LIDIVDQLKNYVNDLVPEFLPAPEDVETGCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_5543 | A112P, R114G | MRSSPGNMERIVICLMVIFLGTLVHKSSSQGQDRHMIRMRQ | 2.081 | 2331 |
| | T121E | LIDIVDQLKNYVNDLVPEFLPAPEDVETNCEWSAFSCFQKA | | |
| | | QLKSANTGNNERIINVSIKKLKRKPPSTNPGGRQKHRLECP | | |
| | | SCDSYEKKPPKEFLERFKSLLQKMIHQHLSSRTHGSEDS | | |
| IL-21_6337 | H35F, I45T, | MRSSPGNMERIVICLMVIFLGTLVHKSSSQGQDRFMIRMRQ | 2.089 | 2332 |
| | N70G | LIDTVDQLKNYVNDLVPEFLPAPEDVETGCEWSAFSCFQKA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_5792 | A112P, H118D | MRSSPGNMERIVICLMVIFLGTLVHKSSSQGQDRHMIRMRQ | 2.099 | 2333 |
| | | LIDIVDQLKNYVNDLVPEFLPAPEDVETNCEWSAFSCFQKA | | |
| | | QLKSANTGNNERIINVSIKKLKRKPPSTNPGRRQKDRLTCP | | |
| | | SCDSYEKKPPKEFLERFKSLLQKMIHQHLSSRTHGSEDS | | |
| IL-21_6258 | I45T, P107V, | MRSSPGNMERIVICLMVIFLGTLVHKSSSQGQDRHMIRMRQ | 2.108 | 2334 |
| | R114G | LIDTVDQLKNYVNDLVPEFLPAPEDVETNCEWSAFSCFQKA | | |
| | | QLKSANTGNNERIINVSIKKLKRKVPSTNAGGRQKHRLTCP | | |
| | | SCDSYEKKPPKEFLERFKSLLQKMIHQHLSSRTHGSEDS | | |
| IL-21_6092 | H35F, I45T, | MRSSPGNMERIVICLMVIFLGTLVHKSSSQGQDRFMIRMRQ | 2.109 | 2335 |
| | N70G, A112P | LIDTVDQLKNYVNDLVPEFLPAPEDVETGCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_6302 | H35F, P107V, | MRSSPGNMERIVICLMVIFLGTLVHKSSSQGQDRFMIRMRQ | 2.12 | 2336 |
| | A112P | LIDIVDQLKNYVNDLVPEFLPAPEDVETNCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_5843 | N70G, P107V | MRSSPGNMERIVICLMVIFLGTLVHKSSSQGQDRHMIRMRQ | 2.127 | 2337 |
| | | LIDIVDQLKNYVNDLVPEFLPAPEDVETGCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_6412 | H35I, R115G | MRSSPGNMERIVICLMVIFLGTLVHKSSSQGQDRIMIRMRQ | 2.135 | 2338 |
| | | LIDIVDQLKNYVNDLVPEFLPAPEDVETNCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_6066 | H35F, I45T, | MRSSPGNMERIVICLMVIFLGTLVHKSSSQGQDREMIRMRQ | 2.138 | 2339 |
| | T110D, A112P | LIDTVDQLKNYVNDLVPEFLPAPEDVETNCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5836 | N70G, R119T | MRSSPGNMERIVICLMVIFLGTLVHKSSSQGQDRHMIRMRQ | 2.144 | 2340 |
| | | LIDIVDQLKNYVNDLVPEFLPAPEDVETGCEWSAFSCFQKA | | |
| | | QLKSANTGNNERIINVSIKKLKRKPPSTNAGRRQKHTLTCP | | |
| | | SCDSYEKKPPKEFLERFKSLLQKMIHQHLSSRTHGSEDS | | |
| IL-21_5806 | P107V, R119T | MRSSPGNMERIVICLMVIFLGTLVHKSSSQGQDRHMIRMRQ | 2.15 | 2341 |
| | | LIDIVDQLKNYVNDLVPEFLPAPEDVETNCEWSAFSCFQKA | | |
| | | QLKSANTGNNERIINVSIKKLKRKVPSTNAGRRQKHTLTCP | | |
| | | SCDSYEKKPPKEFLERFKSLLQKMIHQHLSSRTHGSEDS | | |
| IL-21_6094 | H35F, I45T, | MRSSPGNMERIVICLMVIFLGTLVHKSSSQGQDREMIRMRQ | 2.155 | 2342 |
| | N07G, P107V | LIDTVDQLKNYVNDLVPEFLPAPEDVETGCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_6249 | I45T, A112P, | MRSSPGNMERIVICLMVIFLGTLVHKSSSQGQDRHMIRMRQ | 2.165 | 2343 |
| | R114G | LIDTVDQLKNYVNDLVPEFLPAPEDVETNCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_5598 | P107V, A112P, | MRSSPGNMERIVICLMVIFLGTLVHKSSSQGQDRHMIRMRQ | 2.167 | 2344 |
| | R119T | LIDIVDQLKNYVNDLVPEFLPAPEDVETNCEWSAFSCFQKA | | |
| | | QLKSANTGNNERIINVSIKKLKRKVPSTNPGRRQKHTLTCP | | |
| | | SCDSYEKKPPKEFLERFKSLLQKMIHQHLSSRTHGSEDS | | |
| IL-21_6234 | H35I, I45T, | MRSSPGNMERIVICLMVIFLGTLVHKSSSQGQDRIMIRMRQ | 2.168 | 2345 |
| | N70G, R115G | LIDTVDQLKNYVNDLVPEFLPAPEDVETGCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5835 | N70G, T121E | MRSSPGNMERIVICLMVIFLGTLVHKSSSQGQDRHMIRMRQ | 2.18 | 2346 |
| | | LIDIVDQLKNYVNDLVPEFLPAPEDVETGCEWSAFSCFQKA | | |
| | | QLKSANTGNNERIINVSIKKLKRKPPSTNAGRRQKHRLECP | | |
| | | SCDSYEKKPPKEFLERFKSLLQKMIHQHLSSRTHGSEDS | | |
| IL-21_6387 | H35I, I45T, | MRSSPGNMERIVICLMVIFLGTLVHKSSSQGQDRIMIRMRQ | 2.192 | 2347 |
| | P107V | LIDTVDQLKNYVNDLVPEFLPAPEDVETNCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5548 | A112P, R114Q, | MRSSPGNMERIVICLMVIFLGTLVHKSSSQGQDRHMIRMRQ | 2.193 | 2348 |
| | T121E | LIDIVDQLKNYVNDLVPEFLPAPEDVETNCEWSAFSCFQKA | | |
| | | QLKSANTGNNERIINVSIKKLKRKPPSTNPGQRQKHRLECP | | |
| | | SCDSYEKKPPKEFLERFKSLLQKMIHQHLSSRTHGSEDS | | |
| IL-21_5780 | R114G, T121E | MRSSPGNMERIVICLMVIFLGTLVHKSSSQGQDRHMIRMRQ | 2.193 | 2349 |
| | | LIDIVDQLKNYVNDLVPEFLPAPEDVETNCEWSAFSCFQKA | | |
| | | QLKSANTGNNERIINVSIKKLKRKPPSTNAGGRQKHRLECP | | |
| | | SCDSYEKKPPKEFLERFKSLLQKMIHQHLSSRTHGSEDS | | |
| IL-21_5597 | P107V, A112P, | MRSSPGNMERIVICLMVIFLGTLVHKSSSQGQDRHMIRMRQ | 2.203 | 2350 |
| | T121E | LIDIVDQLKNYVNDLVPEFLPAPEDVETNCEWSAFSCFQKA | | |
| | | QLKSANTGNNERIINVSIKKLKRKVPSTNPGRRQKHRLECP | | |
| | | SCDSYEKKPPKEFLERFKSLLQKMIHQHLSSRTHGSEDS | | |
| IL-21_6037 | H35F, N70G, | MRSSPGNMERIVICLMVIFLGTLVHKSSSQGQDRFMIRMRQ | 2.207 | 2351 |
| | P107V, A112P | LIDIVDQLKNYVNDLVPEFLPAPEDVETGCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_6414 | H35I, R114G | MRSSPGNMERIVICLMVIFLGTLVHKSSSQGQDRIMIRMRQ | 2.214 | 2352 |
| | | LIDIVDQLKNYVNDLVPEFLPAPEDVETNCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |
| IL-21_6276 | I45T, N70G, | MRSSPGNMERIVICLMVIFLGTLVHKSSSQGQDRHMIRMRQ | 2.214 | 2353 |
| | R115G | LIDTVDQLKNYVNDLVPEFLPAPEDVETGCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_6259 | I45T, P107V, | MRSSPGNMERIVICLMVIFLGTLVHKSSSQGQDRHMIRMRQ | 2.22 | 2354 |
| | R114Q | LIDTVDQLKNYVNDLVPEFLPAPEDVETNCEWSAFSCFQKA | | |
| | | QLKSANTGNNERIINVSIKKLKRKVPSTNAGQRQKHRLTCP | | |
| | | SCDSYEKKPPKEFLERFKSLLQKMIHQHLSSRTHGSEDS | | |
| IL-21_5572 | T110D, A112P, | MRSSPGNMERIVICLMVIFLGTLVHKSSSQGQDRHMIRMRQ | 2.227 | 2355 |
| | T121E | LIDIVDQLKNYVNDLVPEFLPAPEDVETNCEWSAFSCFQKA | | |
| | | QLKSANTGNNERIINVSIKKLKRKPPSDNPGRRQKHRLECP | | |
| | | SCDSYEKKPPKEFLERFKSLLQKMIHQHLSSRTHGSEDS | | |
| IL-21_6282 | I45T, N70G, | MRSSPGNMERIVICLMVIFLGTLVHKSSSQGQDRHMIRMRQ | 2.232 | 2356 |
| | P107V | LIDTVDQLKNYVNDLVPEFLPAPEDVETGCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5398 | N70G, P107V, | MRSSPGNMERIVICLMVIFLGTLVHKSSSQGQDRHMIRMRQ | 2.236 | 2357 |
| | A112P, T121E | LIDIVDQLKNYVNDLVPEFLPAPEDVETGCEWSAFSCFQKA | | |
| | | QLKSANTGNNERIINVSIKKLKRKVPSTNPGRRQKHRLECP | | |
| | | SCDSYEKKPPKEFLERFKSLLQKMIHQHLSSRTHGSEDS | | |
| IL-21_6333 | H35F, I45T, | MRSSPGNMERIVICLMVIFLGTLVHKSSSQGQDREMIRMRQ | 2.244 | 2358 |
| | T110D | LIDTVDQLKNYVNDLVPEFLPAPEDVETNCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_5728 | N70G, A112P, | MRSSPGNMERIVICLMVIFLGTLVHKSSSQGQDRHMIRMRQ | 2.245 | 2359 |
| | R119T | LIDIVDQLKNYVNDLVPEFLPAPEDVETGCEWSAFSCFQKA | | |
| | | QLKSANTGNNERIINVSIKKLKRKPPSTNPGRRQKHTLTCP | | |
| | | SCDSYEKKPPKEFLERFKSLLQKMIHQHLSSRTHGSEDS | | |
| IL-21_6261 | I45T, P107V, | MRSSPGNMERIVICLMVIFLGTLVHKSSSQGQDRHMIRMRQ | 2.245 | 2360 |
| | T110D | LIDTVDQLKNYVNDLVPEFLPAPEDVETNCEWSAFSCFQKA | | |
| | | QLKSANTGNNERIINVSIKKLKRKVPSDNAGRRQKHRLTCP | | |
| | | SCDSYEKKPPKEFLERFKSLLQKMIHQHLSSRTHGSEDS | | |
| IL-21_5534 | A112P, R119T, | MRSSPGNMERIVICLMVIFLGTLVHKSSSQGQDRHMIRMRQ | 2.252 | 2361 |
| | T121E | LIDIVDQLKNYVNDLVPEFLPAPEDVETNCEWSAFSCFQKA | | |
| | | QLKSANTGNNERIINVSIKKLKRKPPSTNPGRRQKHTLECP | | |
| | | SCDSYEKKPPKEFLERFKSLLQKMIHQHLSSRTHGSEDS | | |
| IL-21_6390 | H35I, I45T, | MRSSPGNMERIVICLMVIFLGTLVHKSSSQGQDRIMIRMRQ | 2.259 | 2362 |
| | N70G | LIDTVDQLKNYVNDLVPEFLPAPEDVETGCEWSAFSCFQKA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6405 | H35F, A112P | MRSSPGNMERIVICLMVIFLGTLVHKSSSQGQDRFMIRMRQ | 2.259 | 2363 |
| | | LIDIVDQLKNYVNDLVPEFLPAPEDVETNCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_6250 | I45T, A112P, | MRSSPGNMERIVICLMVIFLGTLVHKSSSQGQDRHMIRMRQ | 2.261 | 2364 |
| | R114Q | LIDTVDQLKNYVNDLVPEFLPAPEDVETNCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5805 | P107V, T121E | MRSSPGNMERIVICLMVIFLGTLVHKSSSQGQDRHMIRMRQ | 2.263 | 2365 |
| | | LIDIVDQLKNYVNDLVPEFLPAPEDVETNCEWSAFSCFQKA | | |
| | | QLKSANTGNNERIINVSIKKLKRKVPSTNAGRRQKHRLECP | | |
| | | SCDSYEKKPPKEFLERFKSLLQKMIHQHLSSRTHGSEDS | | |
| IL-21_6342 | H35I, A112P, | MRSSPGNMERIVICLMVIFLGTLVHKSSSQGQDRIMIRMRQ | 2.269 | 2366 |
| | R115G | LIDIVDQLKNYVNDLVPEFLPAPEDVETNCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_6247 | I45T, A112P, | MRSSPGNMERIVICLMVIFLGTLVHKSSSQGQDRHMIRMRQ | 2.272 | 2367 |
| | R115G | LIDTVDQLKNYVNDLVPEFLPAPEDVETNCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5793 | A112P, R115G | MRSSPGNMERIVICLMVIFLGTLVHKSSSQGQDRHMIRMRQ | 2.273 | 2368 |
| | | LIDIVDQLKNYVNDLVPEFLPAPEDVETNCEWSAFSCFQKA | | |
| | | QLKSANTGNNERIINVSIKKLKRKPPSTNPGRGQKHRLTCP | | |
| | | SCDSYEKKPPKEFLERFKSLLQKMIHQHLSSRTHGSEDS | | |
| IL-21_5772 | R119T, T121E | MRSSPGNMERIVICLMVIFLGTLVHKSSSQGQDRHMIRMRQ | 2.274 | 2369 |
| | | LIDIVDQLKNYVNDLVPEFLPAPEDVETNCEWSAFSCFQKA | | |
| | | QLKSANTGNNERIINVSIKKLKRKPPSTNAGRRQKHTLECP | | |
| | | SCDSYEKKPPKEFLERFKSLLQKMIHQHLSSRTHGSEDS | | |
| IL-21_5727 | N70G, A112P, | MRSSPGNMERIVICLMVIFLGTLVHKSSSQGQDRHMIRMRQ | 2.282 | 2370 |
| | T121E | LIDIVDQLKNYVNDLVPEFLPAPEDVETGCEWSAFSCFQKA | | |
| | | QLKSANTGNNERIINVSIKKLKRKPPSTNPGRRQKHRLECP | | |
| | | SCDSYEKKPPKEFLERFKSLLQKMIHQHLSSRTHGSEDS | | |
| IL-21_5928 | I45T, N70G, | MRSSPGNMERIVICLMVIFLGTLVHKSSSQGQDRHMIRMRQ | 2.294 | 2371 |
| | T110D, A112P | LIDTVDQLKNYVNDLVPEFLPAPEDVETGCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_6417 | H35I, T110D | MRSSPGNMERIVICLMVIFLGTLVHKSSSQGQDRIMIRMRQ | 2.314 | 2372 |
| | | LIDIVDQLKNYVNDLVPEFLPAPEDVETNCEWSAFSCFQKA | | |
| | | QLKSANTGNNERIINVSIKKLKRKPPSDNAGRRQKHRLTCP | | |
| | | SCDSYEKKPPKEFLERFKSLLQKMIHQHLSSRTHGSEDS | | |
| IL-21_6334 | H35F, I45T, | MRSSPGNMERIVICLMVIFLGTLVHKSSSQGQDRFMIRMRQ | 2.329 | 2373 |
| | P107V | LIDTVDQLKNYVNDLVPEFLPAPEDVETNCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_6394 | I45T, R114Q | MRSSPGNMERIVICLMVIFLGTLVHKSSSQGQDRHMIRMRQ | 2.333 | 2374 |
| | | LIDTVDQLKNYVNDLVPEFLPAPEDVETNCEWSAFSCFQKA | | |
| | | QLKSANTGNNERIINVSIKKLKRKPPSTNAGQRQKHRLTCP | | |
| | | SCDSYEKKPPKEFLERFKSLLQKMIHQHLSSRTHGSEDS | | |
| IL-21_5781 | R114G, R119T | MRSSPGNMERIVICLMVIFLGTLVHKSSSQGQDRHMIRMRQ | 2.356 | 2375 |
| | | LIDIVDQLKNYVNDLVPEFLPAPEDVETNCEWSAFSCFQKA | | |
| | | QLKSANTGNNERIINVSIKKLKRKPPSTNAGGRQKHTLTCP | | |
| | | SCDSYEKKPPKEFLERFKSLLQKMIHQHLSSRTHGSEDS | | |
| IL-21_6391 | I45T, R115G | MRSSPGNMERIVICLMVIFLGTLVHKSSSQGQDRHMIRMRQ | 2.384 | 2376 |
| | | LIDTVDQLKNYVNDLVPEFLPAPEDVETNCEWSAFSCFQKA | | |
| | | QLKSANTGNNERIINVSIKKLKRKPPSTNAGRGQKHRLTCP | | |
| | | SCDSYEKKPPKEFLERFKSLLQKMIHQHLSSRTHGSEDS | | |
| IL-21_5857 | I45T, T110D, | MRSSPGNMERIVICLMVIFLGTLVHKSSSQGQDRHMIRMRQ | 2.393 | 2377 |
| | A112P, R114Q | LIDTVDQLKNYVNDLVPEFLPAPEDVETNCEWSAFSCFQKA | | |
| | | QLKSANTGNNERIINVSIKKLKRKPPSDNPGQRQKHRLTCP | | |
| | | SCDSYEKKPPKEFLERFKSLLQKMIHQHLSSRTHGSEDS | | |
| IL-21_6238 | H35I, I45T, | MRSSPGNMERIVICLMVIFLGTLVHKSSSQGQDRIMIRMRQ | 2.403 | 2378 |
| | N70G, A112P | LIDTVDQLKNYVNDLVPEFLPAPEDVETGCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5841 | N70G, A112P | MRSSPGNMERIVICLMVIFLGTLVHKSSSQGQDRHMIRMRQ | 2.411 | 2379 |
| | | LIDIVDQLKNYVNDLVPEFLPAPEDVETGCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_6332 | H35F, I45T, | MRSSPGNMERIVICLMVIFLGTLVHKSSSQGQDRFMIRMRQ | 2.463 | 2380 |
| | A112P | LIDTVDQLKNYVNDLVPEFLPAPEDVETNCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_6217 | H35I, I45T, | MRSSPGNMERIVICLMVIFLGTLVHKSSSQGQDRIMIRMRQ | 2.467 | 2381 |
| | P107V, A112P | LIDTVDQLKNYVNDLVPEFLPAPEDVETNCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_6400 | I45T, N70G | MRSSPGNMERIVICLMVIFLGTLVHKSSSQGQDRHMIRMRQ | 2.486 | 2382 |
| | | LIDTVDQLKNYVNDLVPEFLPAPEDVETGCEWSAFSCFQKA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6255 | I45T, T110D, | MRSSPGNMERIVICLMVIFLGTLVHKSSSQGQDRHMIRMRQ | 2.543 | 2383 |
| | A112P | LIDTVDQLKNYVNDLVPEFLPAPEDVETNCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_5804 | T110D, A112P | MRSSPGNMERIVICLMVIFLGTLVHKSSSQGQDRHMIRMRQ | 2.582 | 2384 |
| | | LIDIVDQLKNYVNDLVPEFLPAPEDVETNCEWSAFSCFQKA | | |
| | | QLKSANTGNNERIINVSIKKLKRKPPSDNPGRRQKHRLTCP | | |
| | | SCDSYEKKPPKEFLERFKSLLQKMIHQHLSSRTHGSEDS | | |
| IL-21_6411 | H35F, I45T | MRSSPGNMERIVICLMVIFLGTLVHKSSSQGQDRFMIRMRQ | 2.582 | 2385 |
| | | LIDTVDQLKNYVNDLVPEFLPAPEDVETNCEWSAFSCFQKA | | |
| | | QLKSANTGNNERIINVSIKKLKRKPPSTNAGRRQKHRLTCP | | |
| | | SCDSYEKKPPKEFLERFKSLLQKMIHQHLSSRTHGSEDS | | |
| IL-21_6071 | H35F, I45T, | MRSSPGNMERIVICLMVIFLGTLVHKSSSQGQDREMIRMRQ | 2.588 | 2386 |
| | P107V, A112P | LIDTVDQLKNYVNDLVPEFLPAPEDVETNCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_6418 | H35I, P107V | MRSSPGNMERIVICLMVIFLGTLVHKSSSQGQDRIMIRMRQ | 2.59 | 2387 |
| | | LIDIVDQLKNYVNDLVPEFLPAPEDVETNCEWSAFSCFQKA | | |
| | | QLKSANTGNNERIINVSIKKLKRKVPSTNAGRRQKHRLTCP | | |
| | | SCDSYEKKPPKEFLERFKSLLQKMIHQHLSSRTHGSEDS | | |
| IL-21_5796 | A112P, R114Q | MRSSPGNMERIVICLMVIFLGTLVHKSSSQGQDRHMIRMRQ | 2.596 | 2388 |
| | | LIDIVDQLKNYVNDLVPEFLPAPEDVETNCEWSAFSCFQKA | | |
| | | QLKSANTGNNERIINVSIKKLKRKPPSTNPGQRQKHRLTCP | | |
| | | SCDSYEKKPPKEFLERFKSLLQKMIHQHLSSRTHGSEDS | | |
| IL-21_5795 | A112P, R114G | MRSSPGNMERIVICLMVIFLGTLVHKSSSQGQDRHMIRMRQ | 2.616 | 2389 |
| | | LIDIVDQLKNYVNDLVPEFLPAPEDVETNCEWSAFSCFQKA | | |
| | | QLKSANTGNNERIINVSIKKLKRKPPSTNPGGRQKHRLTCP | | |
| | | SCDSYEKKPPKEFLERFKSLLQKMIHQHLSSRTHGSEDS | | |
| IL-21_6260 | I45T, P107V, | MRSSPGNMERIVICLMVIFLGTLVHKSSSQGQDRHMIRMRQ | 2.673 | 2390 |
| | A112P | LIDTVDQLKNYVNDLVPEFLPAPEDVETNCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_6416 | H35I, A112P | MRSSPGNMERIVICLMVIFLGTLVHKSSSQGQDRIMIRMRQ | 2.695 | 2391 |
| | | LIDIVDQLKNYVNDLVPEFLPAPEDVETNCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5797 | T110D, T121E | MRSSPGNMERIVICLMVIFLGTLVHKSSSQGQDRHMIRMRQ | 2.703 | 2392 |
| | | LIDIVDQLKNYVNDLVPEFLPAPEDVETNCEWSAFSCFQKA | | |
| | | QLKSANTGNNERIINVSIKKLKRKPPSDNAGRRQKHRLECP | | |
| | | SCDSYEKKPPKEFLERFKSLLQKMIHQHLSSRTHGSEDS | | |
| IL-21_6355 | H35I, P107V, | MRSSPGNMERIVICLMVIFLGTLVHKSSSQGQDRIMIRMRQ | 2.704 | 2393 |
| | A112P | LIDIVDQLKNYVNDLVPEFLPAPEDVETNCEWSAFSCFQKA | | |
| | | QLKSANTGNNERIINVSIKKLKRKVPSTNPGRRQKHRLTCP | | |
| | | SCDSYEKKPPKEFLERFKSLLQKMIHQHLSSRTHGSEDS | | |
| IL-21_6385 | H35I, I45T | MRSSPGNMERIVICLMVIFLGTLVHKSSSQGQDRIMIRMRQ | 2.72 | 2394 |
| | A112F | LIDTVDQLKNYVNDLVPEFLPAPEDVETNCEWSAFSCFQKA | | |
| | | QLKSANTGNNERIINVSIKKLKRKPPSTNPGRRQKHRLTCP | | |
| | | SCDSYEKKPPKEFLERFKSLLQKMIHQHLSSRTHGSEDS | | |
| IL-21_5791 | A112P, R119T | MRSSPGNMERIVICLMVIFLGTLVHKSSSQGQDRHMIRMRQ | 2.72 | 2395 |
| | | LIDIVDQLKNYVNDLVPEFLPAPEDVETNCEWSAFSCFQKA | | |
| | | QLKSANTGNNERIINVSIKKLKRKPPSTNPGRRQKHTLTCP | | |
| | | SCDSYEKKPPKEFLERFKSLLQKMIHQHLSSRTHGSEDS | | |
| IL-21_6393 | I45T, R114G | MRSSPGNMERIVICLMVIFLGTLVHKSSSQGQDRHMIRMRQ | 2.72 | 2396 |
| | | LIDTVDQLKNYVNDLVPEFLPAPEDVETNCEWSAFSCFQKA | | |
| | | QLKSANTGNNERIINVSIKKLKRKPPSTNAGGRQKHRLTCP | | |
| | | SCDSYEKKPPKEFLERFKSLLQKMIHQHLSSRTHGSEDS | | |

A further set of 24 stabilized variants identified using AlphaSeq™ was produced as Fe fusion proteins in ExpiCHO™ cells. Titers and analytical SEC percent main peaks for this set of variants is shown in Table 11 below. As with the first iteration of stabilized variants, the second tested set showed a range of purities and titers, with the top variants demonstrating >200 ug/mL titer and >90% purity by analytical SEC.

TABLE 11
Protein Expression Yields and Purity for Second
Iteration Stabilized IL-21-Fc Fusion Proteins

| Variant | Titer (ug/mL) | Percent main peak | SEQ ID NO: |
|---|---|---|---|
| IL-21_5272 | 120.0 | 81.5 | 892 |
| IL-21_6018 | 241.8 | 93.0 | 894 |
| IL-21_5120 | 128.5 | 45.0 | 905 |
| IL-21_5707 | 174.7 | 86.6 | 933 |
| IL-21_5997 | 123.5 | 78.9 | 930 |
| IL-21_5702 | 178.8 | 73.8 | 923 |
| IL-21_6318 | 217.0 | 93.4 | 950 |
| IL-21_5706 | 239.7 | 56.8 | 925 |
| IL-21_5999 | 239.1 | 82.8 | 958 |
| IL-21_5474 | 133.9 | 97.8 | 964 |
| IL-21_5834 | 218.3 | 65.1 | 996 |
| IL-21_6312 | 217.5 | 82.7 | 1025 |
| IL-21_5765 | 298.2 | 93.3 | 1192 |
| IL-21_5818 | 117.3 | 59.4 | 1201 |
| IL-21_5662 | 159.0 | 43.6 | 1218 |
| IL-21_6365 | 123.2 | 36.2 | 1223 |
| IL-21_5828 | 95.9 | 38.9 | 1253 |
| IL-21_5832 | 103.3 | 27.6 | 1464 |
| IL-21_5816 | 106.5 | 61.2 | 1490 |
| IL-21_5830 | 89.9 | 29.6 | 1501 |
| IL-21_6409 | 92.1 | 68.3 | 1675 |
| IL-21_5826 | 103.7 | 45.9 | 1744 |
| IL-21_5845 | 118.6 | 84.2 | 1698 |
| IL-21_6420 | 107.5 | 28.7 | 1904 |

### Example 5: Identification of Stabilized and Detuned IL-21 Variants Using AlphaSeq™

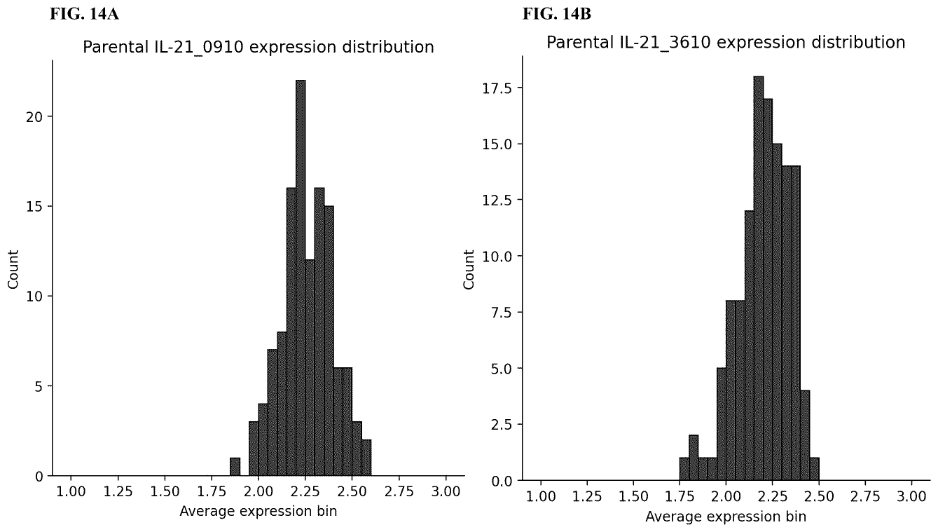

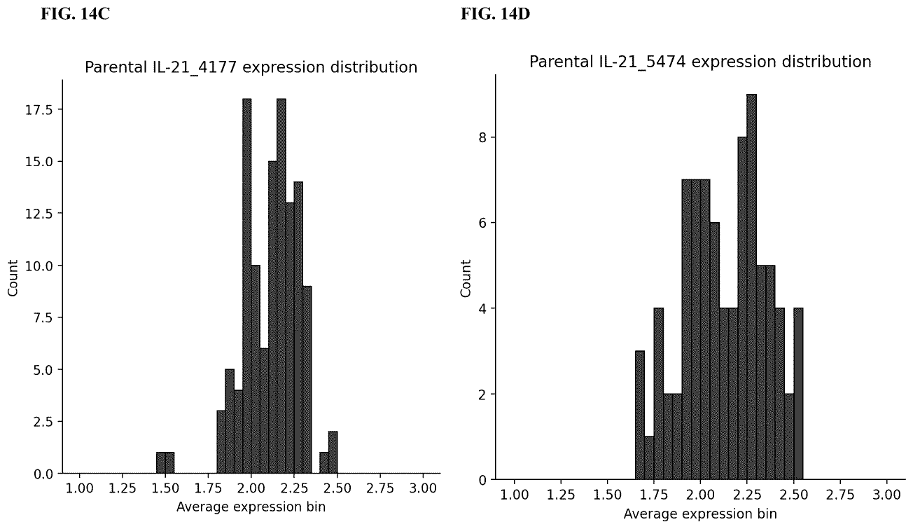

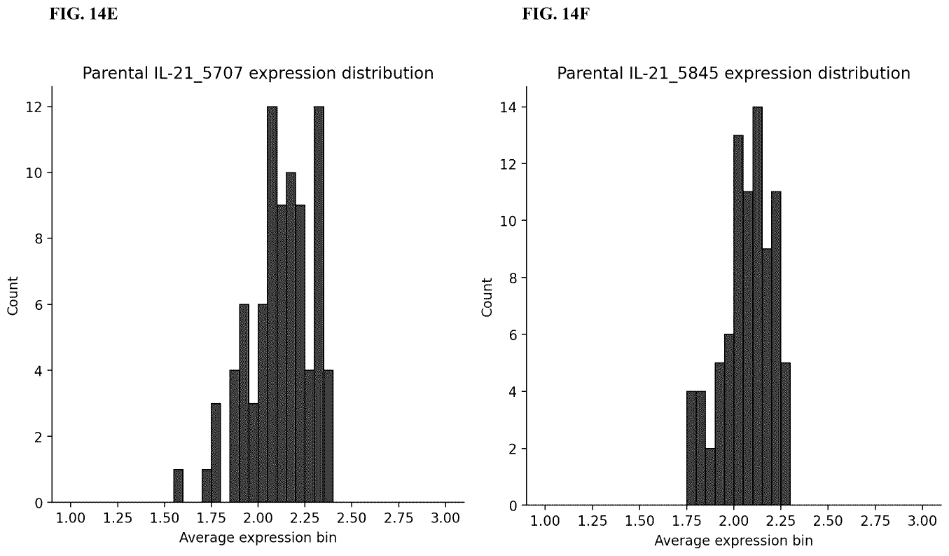

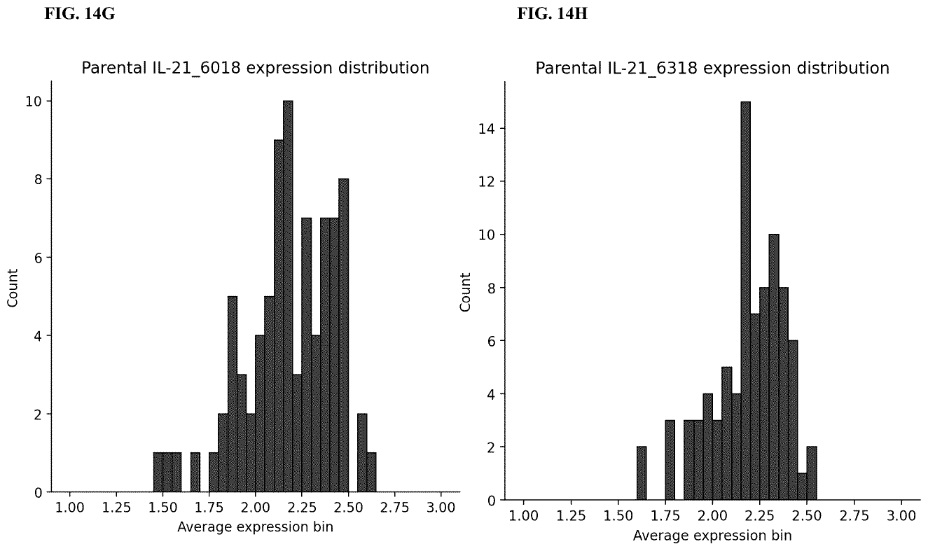

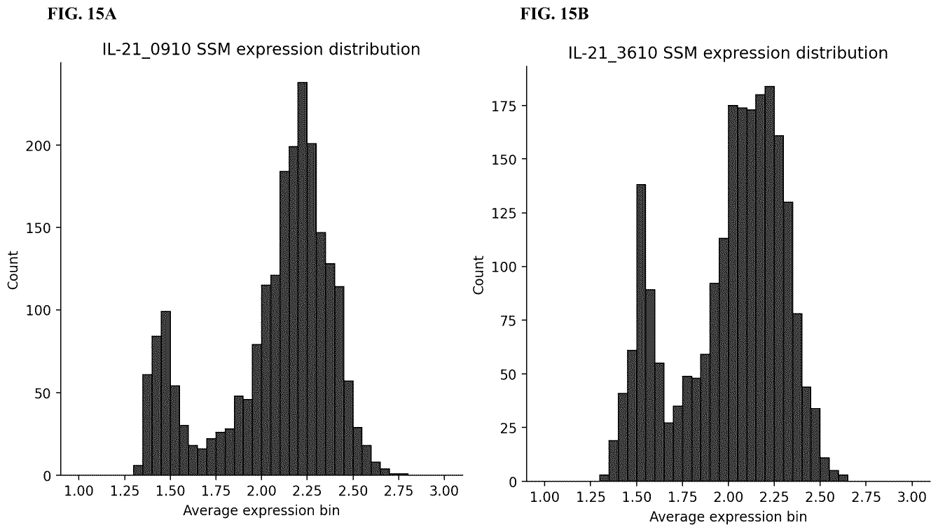

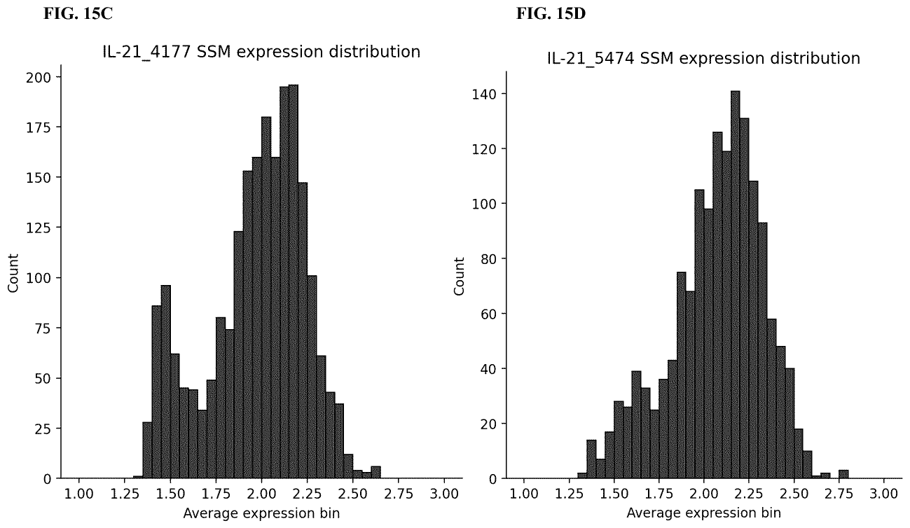

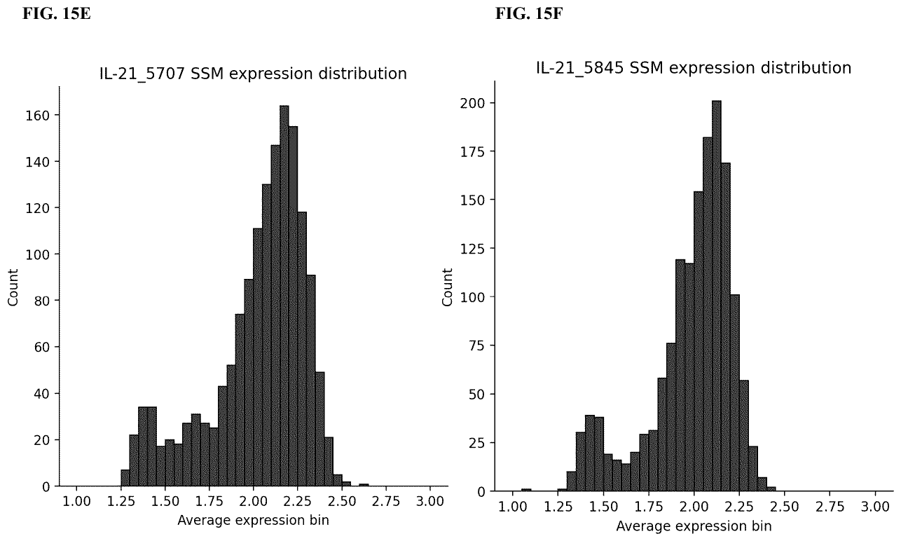

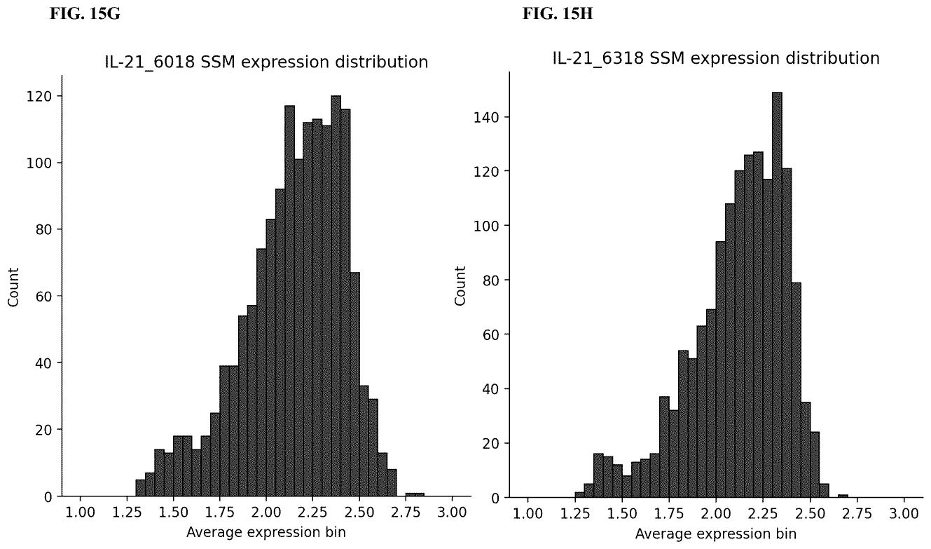

To identify IL-21 variants with both stabilizing and detuning properties, a series of AlphaSeq™ assays were performed in which an SSM library was constructed for each of several stabilized variants identified in the experiments described in EXAMPLE 4. In the first assay, IL-21_0910, IL-21_3610, and IL-21_4177 were used as parental sequences, and a full SSM library was constructed for each parental sequence. In the second assay, IL-21_5474, IL-21_5707, IL-21_5845, IL-21_6018, and IL-21_6318 were selected as parental sequences, and a reduced SSM covering only positions Q32 to R115 in IL-21 was constructed for each parental sequence. A total of 14191 IL-21 variants were designed to be tested across the two assays for binding to human IL-21R. SSM library construction and AlphaSeq™ assays were performed as described in EXAMPLE 2, using unbuffered media. FACS-based expression values were also measured for each variant as described in EXAMPLE 2, with the modification that staining was performed using an antibody recognizing an N-terminal c-myc tag instead of a C-terminal HA tag. The distribution of replicate expression measurements for each of the parental IL-21 sequences is shown in FIG. 14. The distribution of expression values for each SSM library is shown in FIG. 15.

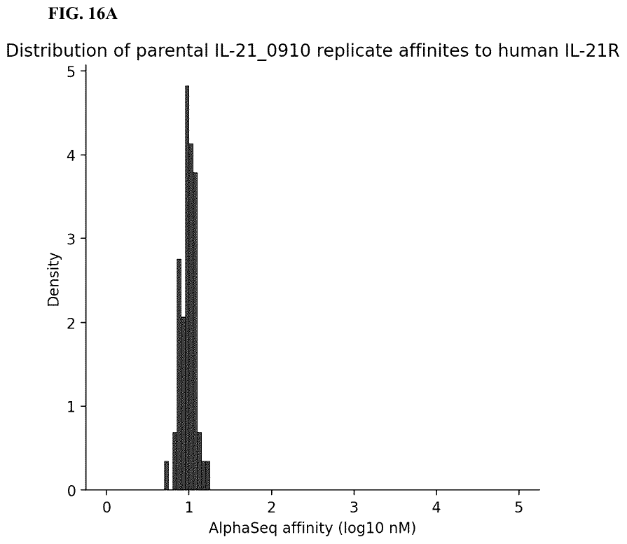

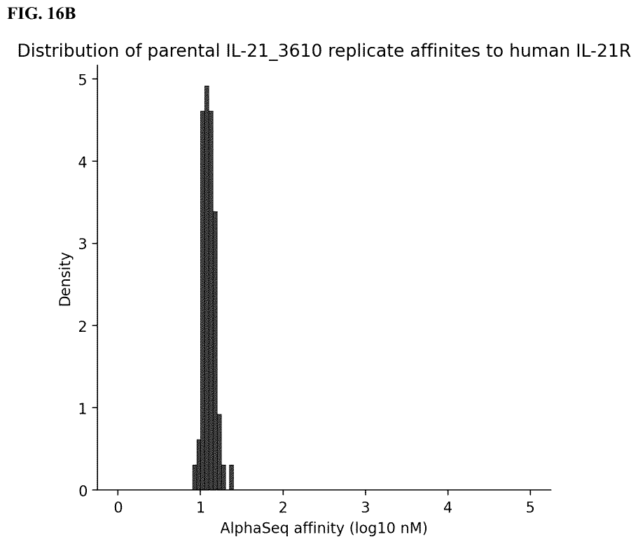

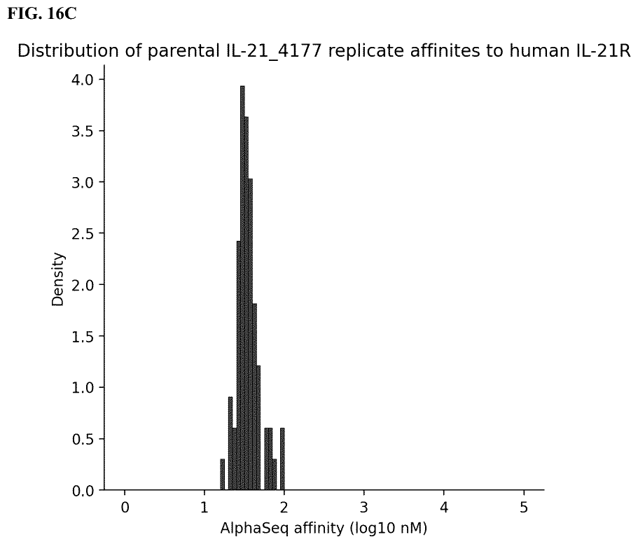

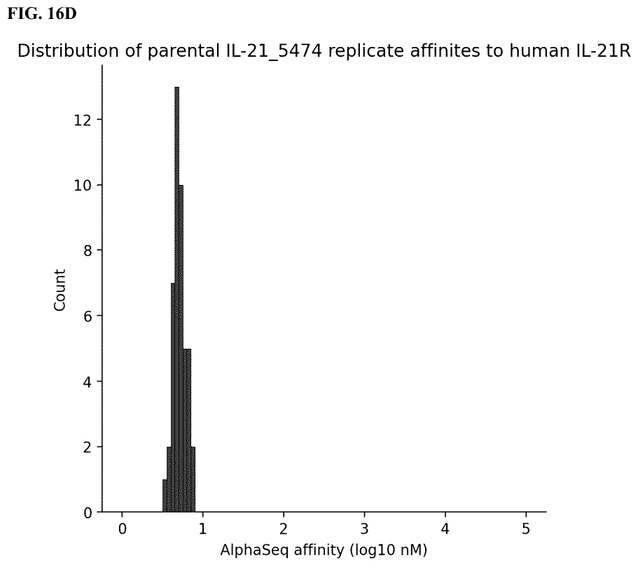

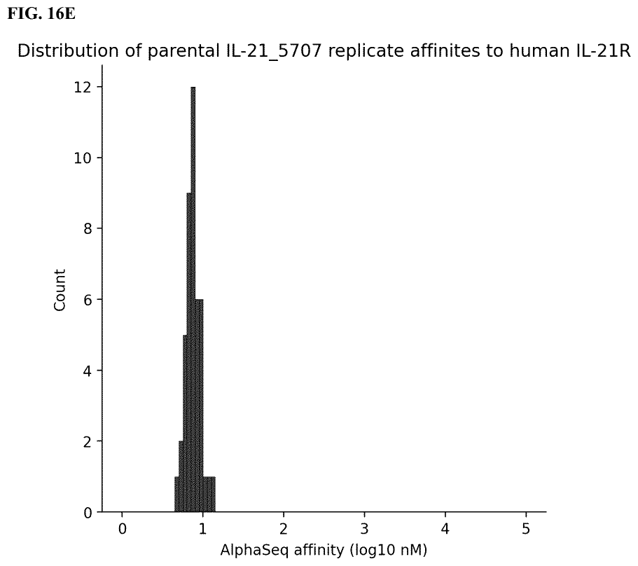

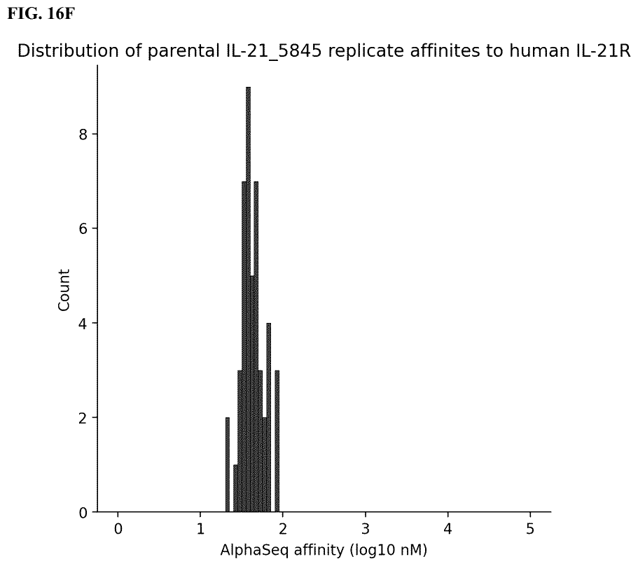

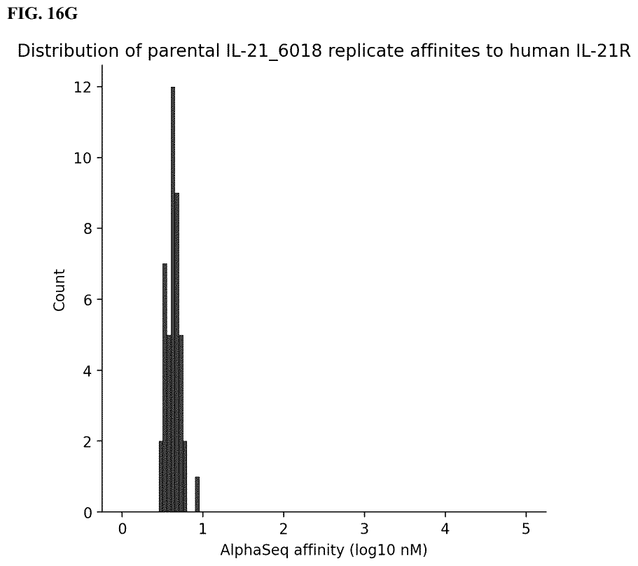

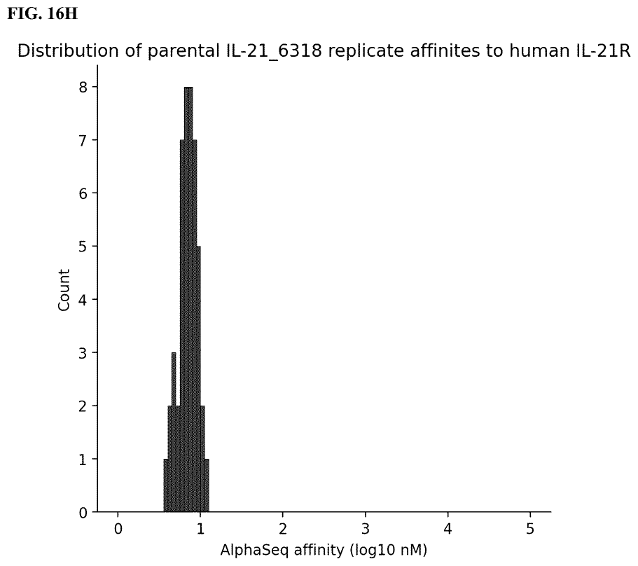

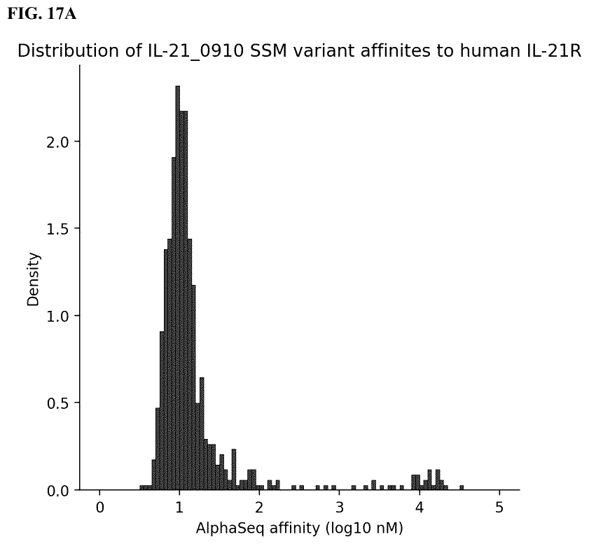

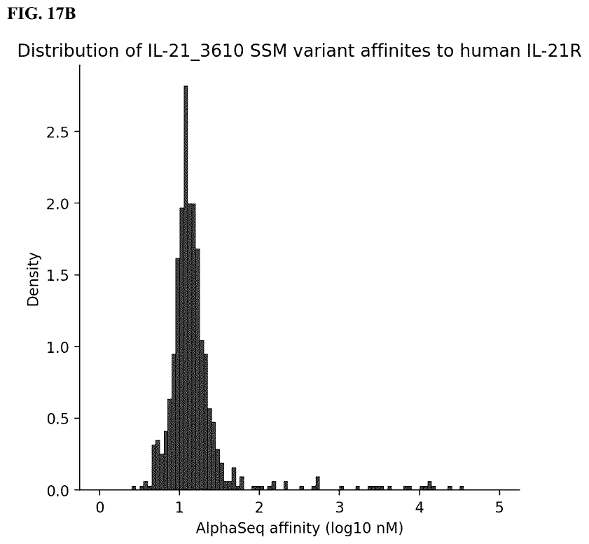

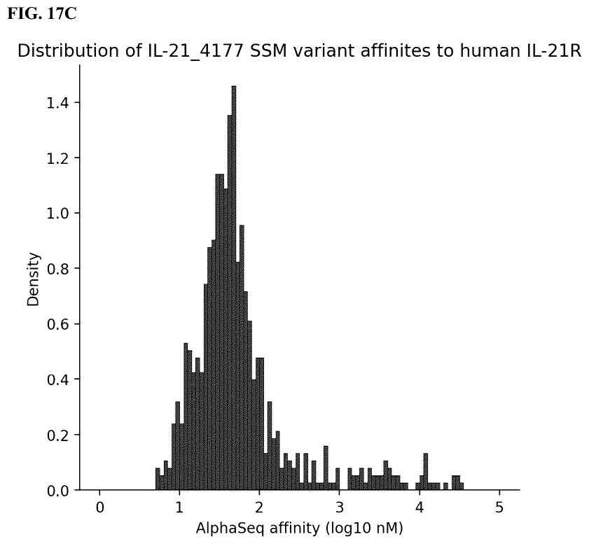

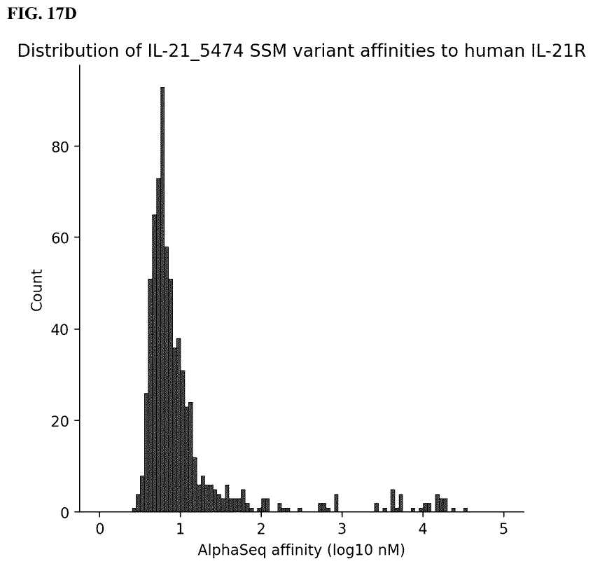

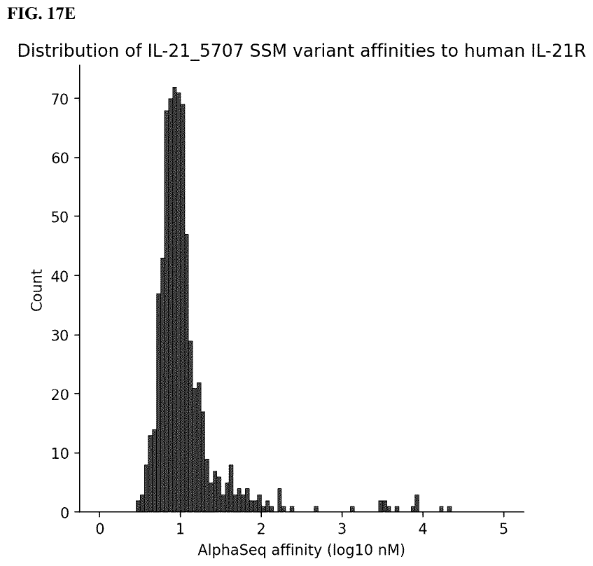

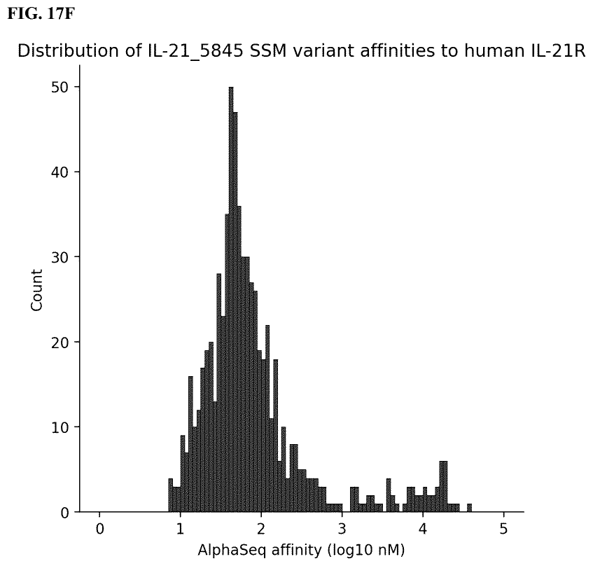

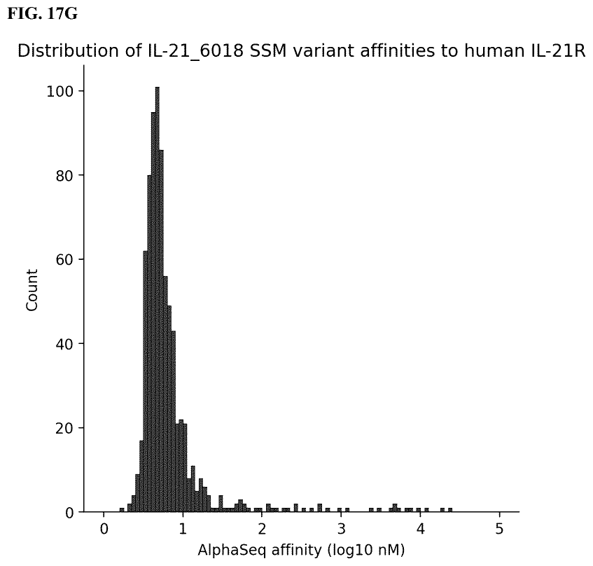

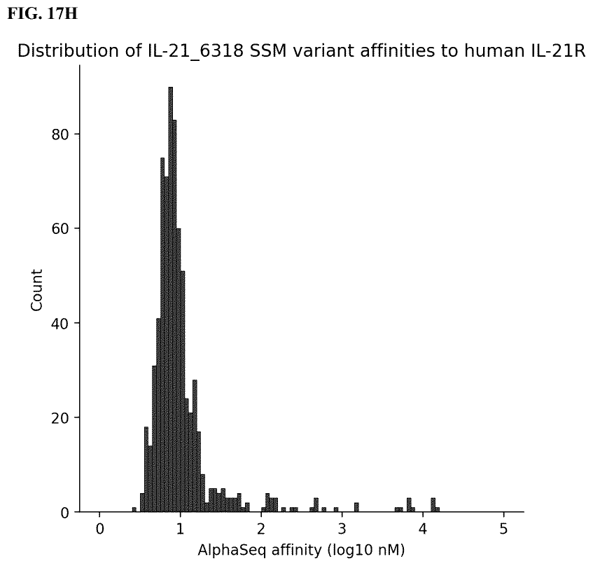

A set of two criteria was used to define detuned variants across the eight mutant libraries. First, as described above in EXAMPLE 2, detuned IL-21 variants were required to have expression values similar to or higher than parental IL-21, to avoid falsely identifying variants with decreased expression or stability as detuned. The expression value cutoff was defined relative to each parental IL-21 (e.g., IL-21_0910, IL-21_3610, and IL-21_4177) as 1 standard deviation less than the average of expression measurements of the parental IL-21. Second, the affinity to human IL-21R had to be at least a half-log reduced relative to the parental IL-21 sequence. The expression and affinity cutoffs for each of the variant libraries is shown in Table 12 below. The AlphaSeq™ replicate affinity distributions for each parental IL-21 sequence, filtered for those meeting the expression cutoff defined above, binding to human IL-21R are shown in FIG. 16. The affinity distributions for each SSM library, filtered for variants meeting the expression cutoff defined above, binding to human IL-21R are shown in FIG. 17.

From the eight mutant libraries tested, a total of 721 detuned stabilized variants were identified. These variants are listed in Table 13 below. The number of times each individual substitution was identified as detuning across the eight stabilized backgrounds are described below. Substitutions that confer detuning in multiple stabilized backgrounds can be considered general detuning substitutions, in contrast to those only identified in one stabilized background, which are more likely to be context-dependent detuning substitutions.

### Substitutions Identified as Detuning in One Stabilized Background:

A112K, A75V, A75Y, A82H, A82I, A82K, A82M, A82T, A82V, A82Y, A87P, D110N, D110R, D110S, D33H, D33P, D33R, D33T, D33V, D33W, D44R, D47F, D47G, D47K, D47R, D55K, D55M, D55R, D55Y, D66K, D81A, D81F, D81I, D81L, D81M, D81Q, D81S, D81W, E59V, E59Y, E65R, E68G, E68R, E72R, E72T, E72W, E93F, E93I, E93K, E93P, E93V, E93Y, E98F, E98G, E98M, E98N, E98P, E98S, E98T, E98V, F76I, F76L, F76M, F76R, F76S, F76V, F79A, F79I, F79L, F79V, G113H, G70M, G70P, G70R, G70W, G90K, G90Y, H118K, I100A, I100E, I100Q, I100Y, I37A, I37Q, I43F, I43R, I43Y, I45F, I45N, I45Y, I95G, I96F, I96G, I96K, I96M, I96N, I96R, K101D, K101E, K102D, K102E, K102G, K102L, K102P, K102T, K104P, K106D, K106G, K106H, K106S, K85P, L103D, L103H, L103I, L103N, L103Q, L103T, L103W, L120S, L144M, L42R, L49F, L56D, L56E, L56F, L56T, L56V, L61K, L84F, L84P, L84T, M36P, M39A, M39P, N111R, N92G, N92P, N92Y, N97H, N97R, N97W, P107Y, P108E, P108H, P108L, P108M, P108Q, P108S, P112R, P58K, P58M, P62L, Q32I, Q41D, Q41E, Q80F, Q80H, Q80I, Q80L, Q80M, Q80S, Q80V, Q83H, Q83K, Q83V, R34A, R34G, R34H, R34I, R34P, R34T, R34Y, R38F, S74K, S74R, S77K, S77R, S77W, S86L, S86M, S86R, S99G, T110H, T45A, T89F, V107A, V107H, V107K, V107R, V53M, V53T, V67G, V67K, V67L, V98F, W73K, Y52G, Y52H, Y52K, Y52N, Y52Q, Y52R, Y52S, and Y52T.

### Substitutions Identified as Detuning in Two Stabilized Backgrounds:

A112R, A82L, A82W, D110H, D44G, D44I, D44K, D44N, D44Q, D44V, D81Y, E93T, E98A, E98H, E98K, E98R, E98Y, G113R, G90I, G90Q, HOOD, I100F, I100G, I100H, I100R, I37F, I37S, I37T, I45G, I45R, I95K, I95R, I96D, I96H, I96P, I96Q, K102I, L103A, L103E, L103F, L103V, L103Y, L42K, L56S, M36R, M39G, N111K, N88R, N92W, N97K, N97P, P107G, P107H, P107K, P108A, P108G, P108N, P108R, Q41P, Q48R, Q80E, Q80G, Q80K, Q80N, Q80W, Q80Y, R105P, R34E, R34W, and S109K.

### Substitutions Identified as Detuning in Three Stabilized Backgrounds:

D44A, D44H, D44L, D44M, D44P, D44W, D81H, D81K, E93S, F76T, I100S, I37K, I37P, I45A, I45K, I45S, I96A, I96E, I96S, I96Y, K102V, L103S, P107R, P108K, P108W, P108Y, Q41K, Q41R, Q41W, Q48K, Q80A, Q80R, Q83F, Q83L, Q83W, R105G, R38G, S99K, and S99P.

### Substitutions Identified as Detuning in Four Stabilized Backgrounds:

D44S, D44T, D81R, E93R, F76K, I37D, I37E, I45Q, I96T, Q80D, R105A, R105E, R105F, R105T, R38E, R38T, S99R, and V98K.

### Substitutions Identified as Detuning in Five Stabilized Backgrounds:

D44F, I37G, I37H, I37N, I37R, R105D, R105H, R105K, R105S, R105V, R34D, R38A, R38P, R38Q, R38S, and R38V.

### Substitutions Identified as Detuning in Six Stabilized Backgrounds:

D44Y, I37W, I45W, R105I, R105M, R105N, R105Q, R38D, R38I, R38K, and R38M.

### Substitutions Identified as Detuning in Seven Stabilized Backgrounds:

R105L, R105W, R105Y, R38L, R38N, and R38W.

TABLE 12
Cutoffs for defining detuned, stabilized variants from AlphaSeq ™

| Parental | Expression | Affinity cutoff | Affinity cutoff | SEQ |
|---|---|---|---|---|
| sequence | cutoff | (nM) | (log₁₀ nM) | ID NO |
| IL-21_0910 | 2.12 | 30.7 | 1.49 | 465 |
| IL-21_3610 | 2.06 | 40.3 | 1.61 | 462 |
| IL-21_4177 | 1.94 | 111.6 | 2.05 | 463 |
| IL-21_5474 | 1.91 | 16.2 | 1.21 | 964 |
| IL-21_5707 | 1.95 | 23.8 | 1.38 | 933 |
| IL-21_5845 | 1.94 | 135.8 | 2.13 | 1698 |
| IL-21_6018 | 1.94 | 13.6 | 1.13 | 894 |
| IL-21_6318 | 1.99 | 22.0 | 1.34 | 950 |

TABLE 13
Detuned IL-21 Variants Identified from Eight Stabilized Parental IL-21 Sequences

| | | | | | SEQ |
|---|---|---|---|---|---|
| | | | | Predicted | ID |
| var_id | parent | mutations | Full_aa_ seq | affinity | NO |
| IL-21_ | IL-21_ | H35F, | MRSSPGNMERIVICLMVIFLGTLVH | 1.141865875 | 2397 |
| 14667 | 6018 | D44Y, | KSSSQGQDRFMIRMRQLIYIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.142647921 | 2398 |
| 15601 | 6018 | K81D, | KSSSQGQDRFMIRMRQLIDIVDQLK | | |
| | | 196T, | NYVNDLVPEFLPAPEDVETNCEWSA | | |
| | | V98E, | FSCFQDAQLKSANTGNNERITNESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.146424431 | 2399 |
| 15575 | 6018 | K81D, | KSSSQGQDRFMIRMRQLIDIVDQLK | | |
| | | 195K, | NYVNDLVPEFLPAPEDVETNCEWSA | | |
| | | V98E, | FSCFQDAQLKSANTGNNERKINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.146491509 | 2400 |
| 15312 | 6018 | Q80R, | KSSSQGQDRFMIRMRQLIDIVDQLK | | |
| | | K81D, | NYVNDLVPEFLPAPEDVETNCEWSA | | |
| | | V98E, | FSCFRDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.149689616 | 2401 |
| 14666 | 6018 | D44W, | KSSSQGQDRFMIRMRQLIWIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.15175483 | 2402 |
| 15385 | 6018 | K81D, | KSSSQGQDRFMIRMRQLIDIVDQLK | | |
| | | L84T, | NYVNDLVPEFLPAPEDVETNCEWSA | | |
| | | V98E, | FSCFQDAQTKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.158552215 | 2403 |
| 14677 | 6018 | I45N, | KSSSQGQDRFMIRMRQLIDNVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.181751989 | 2404 |
| 14338 | 6018 | K81D, | KSSSQGQDRFMIRMRQLIDIVDQLK | | |
| | | V98E, | NYVNDLVPEFLPAPEDVETNCEWSA | | |
| | | T110H | FSCFQDAQLKSANTGNNERIINESI | | |
| | | | KKLKRKPPSHNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.183022098 | 2405 |
| 14668 | 6018 | I45A, | KSSSQGQDRFMIRMRQLIDAVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.190623133 | 2406 |
| 15282 | 6018 | F79A, | KSSSQGQDRFMIRMRQLIDIVDQLK | | |
| | | K81D, | NYVNDLVPEFLPAPEDVETNCEWSA | | |
| | | V98E, | FSCAQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.214196921 | 2407 |
| 20126 | 5474 | K81D, | KSSSQGQDRHMIRMRQLIDIVDQLK | | |
| | | N97K, | NYVNDLVPEFLPAPEDVETGCEWSA | | |
| | | R114Q, | FSCFQDAQLKSANTGNNERIIKVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.216458896 | 2408 |
| 14734 | 6018 | Q48R, | KSSSQGQDRFMIRMRQLIDIVDRLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.221774047 | 2409 |
| 15809 | 6018 | K81D, | KSSSQGQDRFMIRMRQLIDIVDQLK | | |
| | | V98E, | NYVNDLVPEFLPAPEDVETNCEWSA | | |
| | | P108K, | FSCFQDAQLKSANTGNNERIINESI | | |
| | | T110D | KKLKRKPKSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.223326102 | 2410 |
| 14346 | 6018 | K81D, | KSSSQGQDRFMIRMRQLIDIVDQLK | | |
| | | V98E, | NYVNDLVPEFLPAPEDVETNCEWSA | | |
| | | T110R | FSCFQDAQLKSANTGNNERIINESI | | |
| | | | KKLKRKPPSRNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.223946407 | 2411 |
| 15615 | 6018 | K81D, | KSSSQGQDRFMIRMRQLIDIVDQLK | | |
| | | N97P, | NYVNDLVPEFLPAPEDVETNCEWSA | | |
| | | V98E, | FSCFQDAQLKSANTGNNERIIPESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.232401147 | 2412 |
| 14685 | 6018 | I45Y, | KSSSQGQDRFMIRMRQLIDYVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I37T, | MRSSPGNMERIVICLMVIFLGTLVH | 1.235249266 | 2413 |
| 19053 | 5474 | N70G, | KSSSQGQDRHMTRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | Y52K, | MRSSPGNMERIVICLMVIFLGTLVH | 1.238349424 | 2414 |
| 19316 | 5474 | N70G, | KSSSQGQDRHMIRMRQLIDIVDQLK | | |
| | | K81D, | NKVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.238444874 | 2415 |
| 15241 | 6018 | F76T, | KSSSQGQDRFMIRMRQLIDIVDQLK | | |
| | | K81D, | NYVNDLVPEFLPAPEDVETNCEWSA | | |
| | | V98E, | TSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.238712327 | 2416 |
| 14653 | 6018 | D44G, | KSSSQGQDRFMIRMRQLIGIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I37S, | MRSSPGNMERIVICLMVIFLGTLVH | 1.247121451 | 2417 |
| 19052 | 5474 | N70G, | KSSSQGQDRHMSRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.24944817 | 2418 |
| 15071 | 6018 | V67K, | KSSSQGQDRFMIRMRQLIDIVDQLK | | |
| | | K81D, | NYVNDLVPEFLPAPEDKETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.255119474 | 2419 |
| 20282 | 5474 | K81D, | KSSSQGQDRHMIRMRQLIDIVDQLK | | |
| | | K106D, | NYVNDLVPEFLPAPEDVETGCEWSA | | |
| | | R114Q, | FSCFQDAQLKSANTGNNERIINVSI | | |
| | | R115D | KKLKRDPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.260803913 | 2420 |
| 15301 | 6018 | Q80D, | KSSSQGQDRFMIRMRQLIDIVDQLK | | |
| | | K81D, | NYVNDLVPEFLPAPEDVETNCEWSA | | |
| | | V98E, | FSCFDDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.265351538 | 2421 |
| 19738 | 5474 | A75V, | KSSSQGQDRHMIRMRQLIDIVDQLK | | |
| | | K81D, | NYVNDLVPEFLPAPEDVETGCEWSV | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.266310706 | 2422 |
| 19836 | 5474 | K81H, | KSSSQGQDRHMIRMRQLIDIVDQLK | | |
| | | R114Q, | NYVNDLVPEFLPAPEDVETGCEWSA | | |
| | | R115D | FSCFQHAQLKSANTGNNERIINVSI | | |
| | | | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.267316873 | 2423 |
| 20057 | 5474 | K81D, | KSSSQGQDRHMIRMRQLIDIVDQLK | | |
| | | E93P, | NYVNDLVPEFLPAPEDVETGCEWSA | | |
| | | R114Q, | FSCFQDAQLKSANTGNNPRIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.275292788 | 2424 |
| 15653 | 6018 | K81D, | KSSSQGQDRFMIRMRQLIDIVDQLK | | |
| | | V98E, | NYVNDLVPEFLPAPEDVETNCEWSA | | |
| | | S99R, | FSCFQDAQLKSANTGNNERIINERI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.283712661 | 2425 |
| 19857 | 5474 | K81D, | KSSSQGQDRHMIRMRQLIDIVDQLK | | |
| | | A82M, | NYVNDLVPEFLPAPEDVETGCEWSA | | |
| | | R114Q, | FSCFQDMQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R34E, | MRSSPGNMERIVICLMVIFLGTLVH | 1.284462806 | 2426 |
| 14474 | 6018 | H35F, | KSSSQGQDEFMIRMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | Y52G, | MRSSPGNMERIVICLMVIFLGTLVH | 1.284556077 | 2427 |
| 19313 | 5474 | N70G, | KSSSQGQDRHMIRMRQLIDIVDQLK | | |
| | | K81D, | NGVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.285114941 | 2428 |
| 15329 | 6018 | K81R, | KSSSQGQDRFMIRMRQLIDIVDQLK | | |
| | | V98E, | NYVNDLVPEFLPAPEDVETNCEWSA | | |
| | | T110D | FSCFQRAQLKSANTGNNERIINESI | | |
| | | | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | D44A, | MRSSPGNMERIVICLMVIFLGTLVH | 1.286819917 | 2429 |
| 19165 | 5474 | N70G, | KSSSQGQDRHMIRMRQLIAIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.287555273 | 2430 |
| 14716 | 6018 | D47R, | KSSSQGQDRFMIRMRQLIDIVRQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.289622695 | 2431 |
| 15625 | 6018 | K81D, | KSSSQGQDRFMIRMRQLIDIVDQLK | | |
| | | V98F, | NYVNDLVPEFLPAPEDVETNCEWSA | | |
| | | T110D | FSCFQDAQLKSANTGNNERIINESI | | |
| | | | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.293005848 | 2432 |
| 19847 | 5474 | K81W, | KSSSQGQDRHMIRMRQLIDIVDQLK | | |
| | | R114Q, | NYVNDLVPEFLPAPEDVETGCEWSA | | |
| | | R115D | FSCFQWAQLKSANTGNNERIINVSI | | |
| | | | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.303920884 | 2433 |
| 20010 | 5474 | K81D, | KSSSQGQDRHMIRMRQLIDIVDQLK | | |
| | | G90Y, | NYVNDLVPEFLPAPEDVETGCEWSA | | |
| | | R114Q, | FSCFQDAQLKSANTYNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.304372763 | 2434 |
| 15322 | 6018 | K81H, | KSSSQGQDRFMIRMRQLIDIVDQLK | | |
| | | V98E, | NYVNDLVPEFLPAPEDVETNCEWSA | | |
| | | T110D | FSCFQHAQLKSANTGNNERIINESI | | |
| | | | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I37N, | MRSSPGNMERIVICLMVIFLGTLVH | 1.315935377 | 2435 |
| 19048 | 5474 | N70G, | KSSSQGQDRHMNRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.319433734 | 2436 |
| 14558 | 6018 | R38W, | KSSSQGQDRFMIWMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.332413877 | 2437 |
| 19848 | 5474 | K81Y, | KSSSQGQDRHMIRMRQLIDIVDQLK | | |
| | | R114Q, | NYVNDLVPEFLPAPEDVETGCEWSA | | |
| | | R115D | FSCFQYAQLKSANTGNNERIINVSI | | |
| | | | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.333623295 | 2438 |
| 15341 | 6018 | K81D, | KSSSQGQDRFMIRMRQLIDIVDQLK | | |
| | | A82K, | NYVNDLVPEFLPAPEDVETNCEWSA | | |
| | | V98E, | FSCFQDKQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.33412654 | 2439 |
| 19843 | 5474 | K81R, | KSSSQGQDRHMIRMRQLIDIVDQLK | | |
| | | R114Q, | NYVNDLVPEFLPAPEDVETGCEWSA | | |
| | | R115D | FSCFQRAQLKSANTGNNERIINVSI | | |
| | | | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | L56E, | MRSSPGNMERIVICLMVIFLGTLVH | 1.338807053 | 2440 |
| 19383 | 5474 | N70G, | KSSSQGQDRHMIRMRQLIDIVDQLK | | |
| | | K81D, | NYVNDEVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45S, | MRSSPGNMERIVICLMVIFLGTLVH | 1.347240428 | 2441 |
| 19196 | 5474 | N70G, | KSSSQGQDRHMIRMRQLIDSVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.349383072 | 2442 |
| 14879 | 6018 | L56S, | KSSSQGQDRFMIRMRQLIDIVDQLK | | |
| | | K81D, | NYVNDSVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.351202973 | 2443 |
| 20173 | 5474 | K81D, | KSSSQGQDRHMIRMRQLIDIVDQLK | | |
| | | I100A, | NYVNDLVPEFLPAPEDVETGCEWSA | | |
| | | R114Q, | FSCFQDAQLKSANTGNNERIINVSA | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.353932794 | 2444 |
| 20215 | 5474 | K81D, | KSSSQGQDRHMIRMRQLIDIVDQLK | | |
| | | K102I, | NYVNDLVPEFLPAPEDVETGCEWSA | | |
| | | R114Q, | FSCFQDAQLKSANTGNNERIINVSI | | |
| | | R115D | KILKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.360107483 | 2445 |
| 15351 | 6018 | K81D, | KSSSQGQDRFMIRMRQLIDIVDQLK | | |
| | | A82W, | NYVNDLVPEFLPAPEDVETNCEWSA | | |
| | | V98E, | FSCFQDWQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.368302268 | 2446 |
| 13409 | 6318 | P58M, | KSSSQGQDRFMIRMRQLIDIVDQLK | | |
| | | K81D, | NYVNDLVMEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.369335913 | 2447 |
| 13039 | 6318 | I37W, | KSSSQGQDRFMWRMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.374591907 | 2448 |
| 14380 | 6318 | K81D, | KSSSQGQDRFMIRMRQLIDIVDQLK | | |
| | | V98E, | NYVNDLVPEFLPAPEDVETNCEWSA | | |
| | | A112R | FSCFQDAQLKSANTGNNERIINESI | | |
| | | | KKLKRKPPSTNRGRRQKHRLTCPSC | | |
| | | | DSYEKKPPKEFLERFKSLLQKMIHQ | | |
| | | | HLSSRTHGSEDS | | |
| IL-21_ | IL-21_ | D47K, | MRSSPGNMERIVICLMVIFLGTLVH | 1.376791122 | 2449 |
| 19225 | 5474 | N70G, | KSSSQGQDRHMIRMRQLIDIVKQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.378488015 | 2450 |
| 13407 | 6318 | P58K, | KSSSQGQDRFMIRMRQLIDIVDQLK | | |
| | | K81D, | NYVNDLVKEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.381511818 | 2451 |
| 18633 | 5707 | V98A, | KSSSQGQDRHMIRMRQLIDIVDQLK | | |
| | | P107V | NYVNDLVPEFLPAPEDVETNCEWSA | | |
| | | | FSCFQDAQLKSANTGNNERIINASI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44T, | MRSSPGNMERIVICLMVIFLGTLVH | 1.382273341 | 2452 |
| 17676 | 5707 | K81D, | KSSSQGQDRHMIRMRQLITIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.385790216 | 2453 |
| 20144 | 5474 | K81D, | KSSSQGQDRHMIRMRQLIDIVDQLK | | |
| | | V98K, | NYVNDLVPEFLPAPEDVETGCEWSA | | |
| | | R114Q, | FSCFQDAQLKSANTGNNERIINKSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.38600227 | 2454 |
| 14323 | 6318 | K81D, | KSSSQGQDRFMIRMRQLIDIVDQLK | | |
| | | V98E, | NYVNDLVPEFLPAPEDVETNCEWSA | | |
| | | S109K | FSCFQDAQLKSANTGNNERIINESI | | |
| | | | KKLKRKPPKTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.386218421 | 2455 |
| 20115 | 5474 | K81D, | KSSSQGQDRHMIRMRQLIDIVDQLK | | |
| | | 196T, | NYVNDLVPEFLPAPEDVETGCEWSA | | |
| | | R114Q, | FSCFQDAQLKSANTGNNERITNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37W, | MRSSPGNMERIVICLMVIFLGTLVH | 1.397477421 | 2456 |
| 19055 | 5474 | N70G, | KSSSQGQDRHMWRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | Q32I, | MRSSPGNMERIVICLMVIFLGTLVH | 1.403740249 | 2457 |
| 12941 | 6318 | H35F, | KSSSQGIDRFMIRMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.404420084 | 2458 |
| 18371 | 5707 | Q83L, | KSSSQGQDRHMIRMRQLIDIVDQLK | | |
| | | V98E, | NYVNDLVPEFLPAPEDVETNCEWSA | | |
| | | P107V | FSCFQDALLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.405097312 | 2459 |
| 18597 | 5707 | 196A, | KSSSQGQDRHMIRMRQLIDIVDQLK | | |
| | | V98E, | NYVNDLVPEFLPAPEDVETNCEWSA | | |
| | | P107V | FSCFQDAQLKSANTGNNERIANESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D47F, | MRSSPGNMERIVICLMVIFLGTLVH | 1.409036741 | 2460 |
| 19221 | 5474 | N70G, | KSSSQGQDRHMIRMRQLIDIVFQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.409171876 | 2461 |
| 20311 | 5474 | K81D, | KSSSQGQDRHMIRMRQLIDIVDQLK | | |
| | | P107R, | NYVNDLVPEFLPAPEDVETGCEWSA | | |
| | | R114Q, | FSCFQDAQLKSANTGNNERIINVSI | | |
| | | R115D | KKLKRKRPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.410019216 | 2462 |
| 18627 | 5707 | N97R, | KSSSQGQDRHMIRMRQLIDIVDQLK | | |
| | | V98E, | NYVNDLVPEFLPAPEDVETNCEWSA | | |
| | | P107V | FSCFQDAQLKSANTGNNERIIRESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | V67G, | MRSSPGNMERIVICLMVIFLGTLVH | 1.410964946 | 2463 |
| 18079 | 5707 | K81D, | KSSSQGQDRHMIRMRQLIDIVDQLK | | |
| | | V98E, | NYVNDLVPEFLPAPEDGETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.414031637 | 2464 |
| 13057 | 6318 | R38W, | KSSSQGQDRFMIWMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.415862315 | 2465 |
| 18719 | 5707 | V98E, | KSSSQGQDRHMIRMRQLIDIVDQLK | | |
| | | K102V, | NYVNDLVPEFLPAPEDVETNCEWSA | | |
| | | P107V | FSCFQDAQLKSANTGNNERIINESI | | |
| | | | KVLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44V, | MRSSPGNMERIVICLMVIFLGTLVH | 1.417572884 | 2466 |
| 17677 | 5707 | K81D, | KSSSQGQDRHMIRMRQLIVIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.420755309 | 2467 |
| 18876 | 5707 | V98E, | KSSSQGQDRHMIRMRQLIDIVDQLK | | |
| | | P107V, | NYVNDLVPEFLPAPEDVETNCEWSA | | |
| | | N111R | FSCFQDAQLKSANTGNNERIINESI | | |
| | | | KKLKRKVPSTRAGRRQKHRLTCPSC | | |
| | | | DSYEKKPPKEFLERFKSLLQKMIHQ | | |
| | | | HLSSRTHGSEDS | | |
| IL-21_ | IL-21_ | L61K, | MRSSPGNMERIVICLMVIFLGTLVH | 1.423181753 | 2468 |
| 19478 | 5474 | N70G, | KSSSQGQDRHMIRMRQLIDIVDQLK | | |
| | | K81D, | NYVNDLVPEFKPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.438081251 | 2469 |
| 19818 | 5474 | Q80G, | KSSSQGQDRHMIRMRQLIDIVDQLK | | |
| | | K81D, | NYVNDLVPEFLPAPEDVETGCEWSA | | |
| | | R114Q, | FSCFGDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.440410333 | 2470 |
| 13028 | 6318 | 137H, | KSSSQGQDRFMHRMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.445347673 | 2471 |
| 13025 | 6318 | I37E, | KSSSQGQDREMERMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.446214488 | 2472 |
| 14672 | 6018 | I45G, | KSSSQGQDRFMIRMRQLIDGVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.448314715 | 2473 |
| 14120 | 6318 | K81D, | KSSSQGQDRFMIRMRQLIDIVDQLK | | |
| | | V98A | NYVNDLVPEFLPAPEDVETNCEWSA | | |
| | | | FSCFQDAQLKSANTGNNERIINASI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.448984601 | 2474 |
| 19803 | 5474 | F79L, | KSSSQGQDRHMIRMRQLIDIVDQLK | | |
| | | K81D, | NYVNDLVPEFLPAPEDVETGCEWSA | | |
| | | R114Q, | FSCLQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.450257264 | 2475 |
| 18636 | 5707 | V98G, | KSSSQGQDRHMIRMRQLIDIVDQLK | | |
| | | P107V | NYVNDLVPEFLPAPEDVETNCEWSA | | |
| | | | FSCFQDAQLKSANTGNNERIINGSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.452694091 | 2476 |
| 14136 | 6318 | K81D, | KSSSQGQDRFMIRMRQLIDIVDQLK | | |
| | | V98Y | NYVNDLVPEFLPAPEDVETNCEWSA | | |
| | | | FSCFQDAQLKSANTGNNERIINYSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.456376867 | 2477 |
| 13154 | 6318 | D44I, | KSSSQGQDRFMIRMRQLIIIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44I, | MRSSPGNMERIVICLMVIFLGTLVH | 1.463486288 | 2478 |
| 17667 | 5707 | K81D, | KSSSQGQDRHMIRMRQLIIIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.466406698 | 2479 |
| 5726 | 5474 | R114Q, | KSSSQGQDRHMIRMRQLIDIVDQLK | | |
| | | R115D | NYVNDLVPEFLPAPEDVETGCEWSA | | |
| | | | FSCFQKAQLKSANTGNNERIINVSI | | |
| | | | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.472491529 | 2480 |
| 13798 | 6318 | Q80D, | KSSSQGQDRFMIRMRQLIDIVDQLK | | |
| | | K81D, | NYVNDLVPEFLPAPEDVETNCEWSA | | |
| | | V98E | FSCFDDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.47279202 | 2481 |
| 20053 | 5474 | K81D, | KSSSQGQDRHMIRMRQLIDIVDQLK | | |
| | | E93K, | NYVNDLVPEFLPAPEDVETGCEWSA | | |
| | | R114Q, | FSCFQDAQLKSANTGNNKRIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.475944771 | 2482 |
| 19831 | 5474 | Q80Y, | KSSSQGQDRHMIRMRQLIDIVDQLK | | |
| | | K81D, | NYVNDLVPEFLPAPEDVETGCEWSA | | |
| | | R114Q, | FSCFYDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | D44K, | MRSSPGNMERIVICLMVIFLGTLVH | 1.482147269 | 2483 |
| 19171 | 5474 | N70G, | KSSSQGQDRHMIRMRQLIKIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37W, | MRSSPGNMERIVICLMVIFLGTLVH | 1.484882695 | 2484 |
| 17552 | 5707 | K81D, | KSSSQGQDRHMWRMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.487888672 | 2485 |
| 15651 | 6018 | K81D, | KSSSQGQDRFMIRMRQLIDIVDQLK | | |
| | | V98E, | NYVNDLVPEFLPAPEDVETNCEWSA | | |
| | | S99P, | FSCFQDAQLKSANTGNNERIINEPI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.48841929 | 2486 |
| 14205 | 6318 | K81D, | KSSSQGQDRFMIRMRQLIDIVDQLK | | |
| | | V98E, | NYVNDLVPEFLPAPEDVETNCEWSA | | |
| | | K102T | FSCFQDAQLKSANTGNNERIINESI | | |
| | | | KTLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.489715311 | 2487 |
| 15240 | 6018 | F76S, | KSSSQGQDRFMIRMRQLIDIVDQLK | | |
| | | K81D, | NYVNDLVPEFLPAPEDVETNCEWSA | | |
| | | V98E, | SSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I45A, | MRSSPGNMERIVICLMVIFLGTLVH | 1.493380021 | 2488 |
| 17680 | 5707 | K81D, | KSSSQGQDRHMIRMRQLIDAVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.493784765 | 2489 |
| 14867 | 6018 | L56D, | KSSSQGQDRFMIRMRQLIDIVDQLK | | |
| | | K81D, | NYVNDDVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.494626816 | 2490 |
| 14552 | 6018 | R38N, | KSSSQGQDRFMINMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.49494524 | 2491 |
| 11826 | 0910 | K81D, | KSSSQGQDRHMIRMRQLIDTVDQLK | | |
| | | E93S, | NYVNDLVPEFLPAPEDVETNCEWSA | | |
| | | V98E | FSCFQDAQLKSANTGNNSRIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.496040626 | 2492 |
| 18642 | 5707 | V98N, | KSSSQGQDRHMIRMRQLIDIVDQLK | | |
| | | P107V | NYVNDLVPEFLPAPEDVETNCEWSA | | |
| | | | FSCFQDAQLKSANTGNNERIINNSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37D, | MRSSPGNMERIVICLMVIFLGTLVH | 1.499191879 | 2493 |
| 17537 | 5707 | K81D, | KSSSQGQDRHMDRMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.500458197 | 2494 |
| 14660 | 6018 | D44P, | KSSSQGQDRFMIRMRQLIPIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.500534358 | 2495 |
| 13151 | 6318 | D44F, | KSSSQGQDRFMIRMRQLIFIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.503749724 | 2496 |
| 12277 | 0910 | E68R, | KSSSQGQDRHMIRMRQLIDTVDQLK | | |
| | | K81D, | NYVNDLVPEFLPAPEDVRTNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.509008208 | 2497 |
| 13032 | 6318 | I37N, | KSSSQGQDRFMNRMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34I, | MRSSPGNMERIVICLMVIFLGTLVH | 1.510447886 | 2498 |
| 12892 | 0910 | I45T, | KSSSQGQDIHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44S, | MRSSPGNMERIVICLMVIFLGTLVH | 1.513808121 | 2499 |
| 17675 | 5707 | K81D, | KSSSQGQDRHMIRMRQLISIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.516229732 | 2500 |
| 14197 | 6318 | K81D, | KSSSQGQDRFMIRMRQLIDIVDQLK | | |
| | | V98E, | NYVNDLVPEFLPAPEDVETNCEWSA | | |
| | | K102I | FSCFQDAQLKSANTGNNERIINESI | | |
| | | | KILKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44H, | MRSSPGNMERIVICLMVIFLGTLVH | 1.518333885 | 2501 |
| 19169 | 5474 | N70G, | KSSSQGQDRHMIRMRQLIHIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | M36R, | MRSSPGNMERIVICLMVIFLGTLVH | 1.518521226 | 2502 |
| 12852 | 0910 | I45T, | KSSSQGQDRHRIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.527290858 | 2503 |
| 20224 | 5474 | K81D, | KSSSQGQDRHMIRMRQLIDIVDQLK | | |
| | | K102V, | NYVNDLVPEFLPAPEDVETGCEWSA | | |
| | | R114Q, | FSCFQDAQLKSANTGNNERIINVSI | | |
| | | R115D | KVLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37H, | MRSSPGNMERIVICLMVIFLGTLVH | 1.527628673 | 2504 |
| 19044 | 5474 | N70G, | KSSSQGQDRHMHRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.527718503 | 2505 |
| 18871 | 5707 | V98E, | KSSSQGQDRHMIRMRQLIDIVDQLK | | |
| | | P107V, | NYVNDLVPEFLPAPEDVETNCEWSA | | |
| | | N111K | FSCFQDAQLKSANTGNNERIINESI | | |
| | | | KKLKRKVPSTKAGRRQKHRLTCPSC | | |
| | | | DSYEKKPPKEFLERFKSLLQKMIHQ | | |
| | | | HLSSRTHGSEDS | | |
| IL-21_ | IL-21_ | I45A, | MRSSPGNMERIVICLMVIFLGTLVH | 1.531957757 | 2506 |
| 12702 | 0910 | K81D, | KSSSQGQDRHMIRMRQLIDAVDQLK | | |
| | | V98E | NYVNDLVPEFLPAPEDVETNCEWSA | | |
| | | | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.53234003 | 2507 |
| 11827 | 0910 | K81D, | KSSSQGQDRHMIRMRQLIDTVDQLK | | |
| | | E93R, | NYVNDLVPEFLPAPEDVETNCEWSA | | |
| | | V98E | FSCFQDAQLKSANTGNNRRIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.534772893 | 2508 |
| 14314 | 6318 | K81D, | KSSSQGQDRFMIRMRQLIDIVDQLK | | |
| | | V98E, | NYVNDLVPEFLPAPEDVETNCEWSA | | |
| | | P108W | FSCFQDAQLKSANTGNNERIINESI | | |
| | | | KKLKRKPWSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.535863014 | 2509 |
| 13178 | 6318 | I45Q, | KSSSQGQDRFMIRMRQLIDQVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.536138695 | 2510 |
| 11560 | 0910 | K81D, | KSSSQGQDRHMIRMRQLIDTVDQLK | | |
| | | V98E, | NYVNDLVPEFLPAPEDVETNCEWSA | | |
| | | P108Q | FSCFQDAQLKSANTGNNERIINESI | | |
| | | | KKLKRKPQSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D33P, | MRSSPGNMERIVICLMVIFLGTLVH | 1.53836819 | 2511 |
| 12906 | 0910 | I45T, | KSSSQGQPRHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.549471445 | 2512 |
| 18646 | 5707 | V98S, | KSSSQGQDRHMIRMRQLIDIVDQLK | | |
| | | P107V | NYVNDLVPEFLPAPEDVETNCEWSA | | |
| | | | FSCFQDAQLKSANTGNNERIINSSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.55071588 | 2513 |
| 20095 | 5474 | K81D, | KSSSQGQDRHMIRMRQLIDIVDQLK | | |
| | | 195R, | NYVNDLVPEFLPAPEDVETGCEWSA | | |
| | | R114Q, | FSCFQDAQLKSANTGNNERRINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | Q80G, | MRSSPGNMERIVICLMVIFLGTLVH | 1.556806268 | 2514 |
| 18314 | 5707 | K81D, | KSSSQGQDRHMIRMRQLIDIVDQLK | | |
| | | V98E, | NYVNDLVPEFLPAPEDVETNCEWSA | | |
| | | P107V | FSCFGDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Q48K, | MRSSPGNMERIVICLMVIFLGTLVH | 1.56151121 | 2515 |
| 17740 | 5707 | K81D, | KSSSQGQDRHMIRMRQLIDIVDKLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37H, | MRSSPGNMERIVICLMVIFLGTLVH | 1.562356041 | 2516 |
| 17541 | 5707 | K81D, | KSSSQGQDRHMHRMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34D, | MRSSPGNMERIVICLMVIFLGTLVH | 1.570789486 | 2517 |
| 12972 | 6318 | H35F, | KSSSQGQDDEMIRMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.572191517 | 2518 |
| 14209 | 6318 | K81D, | KSSSQGQDRFMIRMRQLIDIVDQLK | | |
| | | V98E, | NYVNDLVPEFLPAPEDVETNCEWSA | | |
| | | L103A | FSCFQDAQLKSANTGNNERIINESI | | |
| | | | KKAKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D33H, | MRSSPGNMERIVICLMVIFLGTLVH | 1.573981072 | 2519 |
| 17470 | 5707 | K81D, | KSSSQGQHRHMIRMRQLIDIVDQLK | | |
| | | V98E, | NYVNDLVPEFLPAPEDVETNCEWSA | | |
| | | P107 | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.577110892 | 2520 |
| 19821 | 5474 | Q80K, | KSSSQGQDRHMIRMRQLIDIVDQLK | | |
| | | K81D, | NYVNDLVPEFLPAPEDVETGCEWSA | | |
| | | R114Q, | FSCFKDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37E, | MRSSPGNMERIVICLMVIFLGTLVH | 1.577482361 | 2521 |
| 19041 | 5474 | N70G, | KSSSQGQDRHMERMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R34W, | MRSSPGNMERIVICLMVIFLGTLVH | 1.577997468 | 2522 |
| 19001 | 5474 | N70G, | KSSSQGQDWHMIRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.580018355 | 2523 |
| 12138 | 0910 | F76L, | KSSSQGQDRHMIRMRQLIDTVDQLK | | |
| | | K81D, | NYVNDLVPEFLPAPEDVETNCEWSA | | |
| | | V98E | LSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.582651145 | 2524 |
| 14542 | 6018 | R38A, | KSSSQGQDREMIAMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I45Q, | MRSSPGNMERIVICLMVIFLGTLVH | 1.583475419 | 2525 |
| 17691 | 5707 | K81D, | KSSSQGQDRHMIRMRQLIDQVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34T, | MRSSPGNMERIVICLMVIFLGTLVH | 1.584919005 | 2526 |
| 12884 | 0910 | I45T, | KSSSQGQDTHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.586291424 | 2527 |
| 13170 | 6318 | I45F, | KSSSQGQDRFMIRMRQLIDFVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.586672575 | 2528 |
| 20166 | 5474 | K81D, | KSSSQGQDRHMIRMRQLIDIVDQLK | | |
| | | S99P, | NYVNDLVPEFLPAPEDVETGCEWSA | | |
| | | R114Q, | FSCFQDAQLKSANTGNNERIINVPI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.589590958 | 2529 |
| 11694 | 0910 | K81D, | KSSSQGQDRHMIRMRQLIDTVDQLK | | |
| | | V98E, | NYVNDLVPEFLPAPEDVETNCEWSA | | |
| | | K101E | FSCFQDAQLKSANTGNNERIINESI | | |
| | | | EKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | M39G, | MRSSPGNMERIVICLMVIFLGTLVH | 1.590567897 | 2530 |
| 19079 | 5474 | N70G, | KSSSQGQDRHMIRGRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R34P, | MRSSPGNMERIVICLMVIFLGTLVH | 1.591612429 | 2531 |
| 12887 | 0910 | I45T, | KSSSQGQDPHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.60352385 | 2532 |
| 19817 | 5474 | Q80F, | KSSSQGQDRHMIRMRQLIDIVDQLK | | |
| | | K81D, | NYVNDLVPEFLPAPEDVETGCEWSA | | |
| | | R114Q, | FSCFFDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.604339748 | 2533 |
| 18352 | 5707 | A82L, | KSSSQGQDRHMIRMRQLIDIVDQLK | | |
| | | V98E, | NYVNDLVPEFLPAPEDVETNCEWSA | | |
| | | P107V | FSCFQDLQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | V53T, | MRSSPGNMERIVICLMVIFLGTLVH | 1.607306576 | 2534 |
| 10375 | 3610 | P107V, | KSSSQGQDRHMIRMRQLIDIVDQLK | | |
| | | R114Q, | NYTNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.61694292 | 2535 |
| 18350 | 5707 | A82I, | KSSSQGQDRHMIRMRQLIDIVDQLK | | |
| | | V98E, | NYVNDLVPEFLPAPEDVETNCEWSA | | |
| | | P107V | FSCFQDIQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.62109777 | 2536 |
| 18555 | 5707 | E93R, | KSSSQGQDRHMIRMRQLIDIVDQLK | | |
| | | V98E, | NYVNDLVPEFLPAPEDVETNCEWSA | | |
| | | P107V | FSCFQDAQLKSANTGNNRRIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.622464182 | 2537 |
| 11558 | 0910 | K81D, | KSSSQGQDRHMIRMRQLIDTVDQLK | | |
| | | V98E, | NYVNDLVPEFLPAPEDVETNCEWSA | | |
| | | P108S | FSCFQDAQLKSANTGNNERIINESI | | |
| | | | KKLKRKPSSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.626104963 | 2538 |
| 18641 | 5707 | V98M, | KSSSQGQDRHMIRMRQLIDIVDQLK | | |
| | | P107V | NYVNDLVPEFLPAPEDVETNCEWSA | | |
| | | | FSCFQDAQLKSANTGNNERIINMSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.627234315 | 2539 |
| 14087 | 6318 | K81D, | KSSSQGQDRFMIRMRQLIDIVDQLK | | |
| | | 196F, | NYVNDLVPEFLPAPEDVETNCEWSA | | |
| | | V98E | FSCFQDAQLKSANTGNNERIENESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44Y, | MRSSPGNMERIVICLMVIFLGTLVH | 1.62880169 | 2540 |
| 19182 | 5474 | N70G, | KSSSQGQDRHMIRMRQLIYIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.629057359 | 2541 |
| 18799 | 5707 | V98E, | KSSSQGQDRHMIRMRQLIDIVDQLK | | |
| | | P107H | NYVNDLVPEFLPAPEDVETNCEWSA | | |
| | | | FSCFQDAQLKSANTGNNERIINESI | | |
| | | | KKLKRKHPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.631039002 | 2542 |
| 14168 | 6318 | K81D, | KSSSQGQDRFMIRMRQLIDIVDQLK | | |
| | | V98E, | NYVNDLVPEFLPAPEDVETNCEWSA | | |
| | | I100S | FSCFQDAQLKSANTGNNERIINESS | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.631818856 | 2543 |
| 15704 | 6018 | K81D, | KSSSQGQDRFMIRMRQLIDIVDQLK | | |
| | | V98E, | NYVNDLVPEFLPAPEDVETNCEWSA | | |
| | | K102P, | FSCFQDAQLKSANTGNNERIINESI | | |
| | | T110D | KPLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.632038347 | 2544 |
| 19853 | 5474 | K81D, | KSSSQGQDRHMIRMRQLIDIVDQLK | | |
| | | A82H, | NYVNDLVPEFLPAPEDVETGCEWSA | | |
| | | R114Q, | FSCFQDHQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | F79V, | MRSSPGNMERIVICLMVIFLGTLVH | 1.632416107 | 2545 |
| 18307 | 5707 | K81D, | KSSSQGQDRHMIRMRQLIDIVDQLK | | |
| | | V98E, | NYVNDLVPEFLPAPEDVETNCEWSA | | |
| | | P107V | FSCVQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.633469804 | 2546 |
| 13045 | 6318 | R38G, | KSSSQGQDRFMIGMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44M, | MRSSPGNMERIVICLMVIFLGTLVH | 1.640496046 | 2547 |
| 17670 | 5707 | K81D, | KSSSQGQDRHMIRMRQLIMIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.646210536 | 2548 |
| 12130 | 0910 | F76V, | KSSSQGQDRHMIRMRQLIDTVDQLK | | |
| | | K81D, | NYVNDLVPEFLPAPEDVETNCEWSA | | |
| | | V98E | VSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44L, | MRSSPGNMERIVICLMVIFLGTLVH | 1.646382709 | 2549 |
| 17669 | 5707 | K81D, | KSSSQGQDRHMIRMRQLILIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37W, | MRSSPGNMERIVICLMVIFLGTLVH | 1.653871994 | 2550 |
| 10661 | 3610 | P107V, | KSSSQGQDRHMWRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.655819722 | 2551 |
| 14150 | 6318 | K81D, | KSSSQGQDRFMIRMRQLIDIVDQLK | | |
| | | V98E, | NYVNDLVPEFLPAPEDVETNCEWSA | | |
| | | S99R | FSCFQDAQLKSANTGNNERIINERI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.6578084 | 2552 |
| 14101 | 6318 | K81D, | KSSSQGQDRFMIRMRQLIDIVDQLK | | |
| | | 196Y, | NYVNDLVPEFLPAPEDVETNCEWSA | | |
| | | V98E | FSCFQDAQLKSANTGNNERIYNESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.658597331 | 2553 |
| 11567 | 0910 | K81D, | KSSSQGQDRHMIRMRQLIDTVDQLK | | |
| | | V98E, | NYVNDLVPEFLPAPEDVETNCEWSA | | |
| | | P108G | FSCFQDAQLKSANTGNNERIINESI | | |
| | | | KKLKRKPGSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37D, | MRSSPGNMERIVICLMVIFLGTLVH | 1.661450518 | 2554 |
| 10676 | 3610 | P107V, | KSSSQGQDRHMDRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.663091164 | 2555 |
| 11571 | 0910 | K81D, | KSSSQGQDRHMIRMRQLIDTVDQLK | | |
| | | V98E, | NYVNDLVPEFLPAPEDVETNCEWSA | | |
| | | P108A | FSCFQDAQLKSANTGNNERIINESI | | |
| | | | KKLKRKPASTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.663277441 | 2556 |
| 14530 | 6018 | I37K, | KSSSQGQDRFMKRMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.664919533 | 2557 |
| 12137 | 0910 | F76M, | KSSSQGQDRHMIRMRQLIDTVDQLK | | |
| | | K81D, | NYVNDLVPEFLPAPEDVETNCEWSA | | |
| | | V98E | MSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37N, | MRSSPGNMERIVICLMVIFLGTLVH | 1.670752567 | 2558 |
| 17545 | 5707 | K81D, | KSSSQGQDRHMNRMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38W, | MRSSPGNMERIVICLMVIFLGTLVH | 1.67455096 | 2559 |
| 19073 | 5474 | N70G, | KSSSQGQDRHMIWMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.675654287 | 2560 |
| 13035 | 6318 | I37R, | KSSSQGQDRFMRRMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37T, | MRSSPGNMERIVICLMVIFLGTLVH | 1.6814949 | 2561 |
| 12832 | 0910 | I45T, | KSSSQGQDRHMTRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.681741838 | 2562 |
| 14551 | 6018 | R38M, | KSSSQGQDRFMIMMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.683695788 | 2563 |
| 20129 | 5474 | K81D, | KSSSQGQDRHMIRMRQLIDIVDQLK | | |
| | | N97P, | NYVNDLVPEFLPAPEDVETGCEWSA | | |
| | | R114Q, | FSCFQDAQLKSANTGNNERIIPVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45S, | MRSSPGNMERIVICLMVIFLGTLVH | 1.683769767 | 2564 |
| 10520 | 3610 | P107V, | KSSSQGQDRHMIRMRQLIDSVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.68671297 | 2565 |
| 11674 | 0910 | K81D, | KSSSQGQDRHMIRMRQLIDTVDQLK | | |
| | | V98E, | NYVNDLVPEFLPAPEDVETNCEWSA | | |
| | | K102G | FSCFQDAQLKSANTGNNERIINESI | | |
| | | | KGLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.689515919 | 2566 |
| 11695 | 0910 | K81D, | KSSSQGQDRHMIRMRQLIDTVDQLK | | |
| | | V98E, | NYVNDLVPEFLPAPEDVETNCEWSA | | |
| | | K101D | FSCFQDAQLKSANTGNNERIINESI | | |
| | | | DKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Q41E, | MRSSPGNMERIVICLMVIFLGTLVH | 1.690581322 | 2567 |
| 12772 | 0910 | I45T, | KSSSQGQDRHMIRMRELIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37G, | MRSSPGNMERIVICLMVIFLGTLVH | 1.69194705 | 2568 |
| 17540 | 5707 | K81D, | KSSSQGQDRHMGRMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D55K, | MRSSPGNMERIVICLMVIFLGTLVH | 1.694054594 | 2569 |
| 19369 | 5474 | N70G, | KSSSQGQDRHMIRMRQLIDIVDQLK | | |
| | | K81D, | NYVNKLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.695897592 | 2570 |
| 18379 | 5707 | Q83W, | KSSSQGQDRHMIRMRQLIDIVDQLK | | |
| | | V98E, | NYVNDLVPEFLPAPEDVETNCEWSA | | |
| | | P107V | FSCFQDAWLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.697113245 | 2571 |
| 11797 | 0910 | K81D, | KSSSQGQDRHMIRMRQLIDTVDQLK | | |
| | | I95K, | NYVNDLVPEFLPAPEDVETNCEWSA | | |
| | | V98E | FSCFQDAQLKSANTGNNERKINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | P107V, | MRSSPGNMERIVICLMVIFLGTLVH | 1.699215785 | 2572 |
| 9397 | 3610 | P108H, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVHSTNAGQDQKHRLTCPSC | | |
| | | | DSYEKKPPKEFLERFKSLLQKMIHQ | | |
| | | | HLSSRTHGSEDS | | |
| IL-21_ | IL-21_ | P107V, | MRSSPGNMERIVICLMVIFLGTLVH | 1.699399491 | 2573 |
| 9377 | 3610 | S109K, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPKTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.705571714 | 2574 |
| 15725 | 6018 | K81D, | KSSSQGQDRFMIRMRQLIDIVDQLK | | |
| | | V98E, | NYVNDLVPEFLPAPEDVETNCEWSA | | |
| | | L103S, | FSCFQDAQLKSANTGNNERIINESI | | |
| | | T110D | KKSKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.706755615 | 2575 |
| 14644 | 6018 | I43R, | KSSSQGQDRFMIRMRQLRDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.71033211 | 2576 |
| 13165 | 6318 | D44W, | KSSSQGQDRFMIRMRQLIWIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D33R, | MRSSPGNMERIVICLMVIFLGTLVH | 1.71167565 | 2577 |
| 12965 | 6318 | H35F, | KSSSQGQRRFMIRMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D55R, | MRSSPGNMERIVICLMVIFLGTLVH | 1.714481786 | 2578 |
| 19375 | 5474 | N70G, | KSSSQGQDRHMIRMRQLIDIVDQLK | | |
| | | K81D, | NYVNRLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.725065364 | 2579 |
| 5833 | 5707 | P107V | KSSSQGQDRHMIRMRQLIDIVDQLK | | |
| | | | NYVNDLVPEFLPAPEDVETNCEWSA | | |
| | | | FSCFQDAQLKSANTGNNERIINVSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.733252549 | 2580 |
| 18647 | 5707 | V98T, | KSSSQGQDRHMIRMRQLIDIVDQLK | | |
| | | P107V | NYVNDLVPEFLPAPEDVETNCEWSA | | |
| | | | FSCFQDAQLKSANTGNNERIINTSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.733313396 | 2581 |
| 13167 | 6318 | I45A, | KSSSQGQDRFMIRMRQLIDAVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.73562517 | 2582 |
| 14223 | 6318 | K81D, | KSSSQGQDRFMIRMRQLIDIVDQLK | | |
| | | V98E, | NYVNDLVPEFLPAPEDVETNCEWSA | | |
| | | L103T | FSCFQDAQLKSANTGNNERIINESI | | |
| | | | KKTKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.735966996 | 2583 |
| 20230 | 5474 | K81D, | KSSSQGQDRHMIRMRQLIDIVDQLK | | |
| | | L103F, | NYVNDLVPEFLPAPEDVETGCEWSA | | |
| | | R114Q, | FSCFQDAQLKSANTGNNERIINVSI | | |
| | | R115D | KKEKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | M39A, | MRSSPGNMERIVICLMVIFLGTLVH | 1.736631431 | 2584 |
| 10641 | 3610 | P107V, | KSSSQGQDRHMIRARQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.736920606 | 2585 |
| 18657 | 5707 | V98E, | KSSSQGQDRHMIRMRQLIDIVDQLK | | |
| | | S99K, | NYVNDLVPEFLPAPEDVETNCEWSA | | |
| | | P107V | FSCFQDAQLKSANTGNNERIINEKI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.740073703 | 2586 |
| 12140 | 0910 | F76I, | KSSSQGQDRHMIRMRQLIDTVDQLK | | |
| | | K81D, | NYVNDLVPEFLPAPEDVETNCEWSA | | |
| | | V98E | ISCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.741388477 | 2587 |
| 19830 | 5474 | Q80W, | KSSSQGQDRHMIRMRQLIDIVDQLK | | |
| | | K81D, | NYVNDLVPEFLPAPEDVETGCEWSA | | |
| | | R114Q, | FSCFWDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.745539562 | 2588 |
| 15765 | 6018 | K81D, | KSSSQGQDRFMIRMRQLIDIVDQLK | | |
| | | V98E, | NYVNDLVPEFLPAPEDVETNCEWSA | | |
| | | R105Y, | FSCFQDAQLKSANTGNNERIINESI | | |
| | | T110D | KKLKYKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I45G, | MRSSPGNMERIVICLMVIFLGTLVH | 1.748118414 | 2589 |
| 17684 | 5707 | K81D, | KSSSQGQDRHMIRMRQLIDGVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.750783932 | 2590 |
| 11584 | 0910 | K81D, | KSSSQGQDRHMIRMRQLIDTVDQLK | | |
| | | V98E, | NYVNDLVPEFLPAPEDVETNCEWSA | | |
| | | P107G | FSCFQDAQLKSANTGNNERIINESI | | |
| | | | KKLKRKGPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.75093725 | 2591 |
| 15635 | 6018 | K81D, | KSSSQGQDRFMIRMRQLIDIVDQLK | | |
| | | V98R, | NYVNDLVPEFLPAPEDVETNCEWSA | | |
| | | T110D | FSCFQDAQLKSANTGNNERIINRSI | | |
| | | | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.751116739 | 2592 |
| 13166 | 6318 | D44Y, | KSSSQGQDRFMIRMRQLIYIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.758229766 | 2593 |
| 18806 | 5707 | V98E, | KSSSQGQDRHMIRMRQLIDIVDQLK | | |
| | | P107R | NYVNDLVPEFLPAPEDVETNCEWSA | | |
| | | | FSCFQDAQLKSANTGNNERIINESI | | |
| | | | KKLKRKRPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.760647271 | 2594 |
| 19815 | 5474 | Q80D, | KSSSQGQDRHMIRMRQLIDIVDQLK | | |
| | | K81D, | NYVNDLVPEFLPAPEDVETGCEWSA | | |
| | | R114Q, | FSCFDDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.760945616 | 2595 |
| 15756 | 6018 | K81D, | KSSSQGQDRFMIRMRQLIDIVDQLK | | |
| | | V98E, | NYVNDLVPEFLPAPEDVETNCEWSA | | |
| | | R105L, | FSCFQDAQLKSANTGNNERIINESI | | |
| | | T110D | KKLKLKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.761102517 | 2596 |
| 19747 | 5474 | F76K, | KSSSQGQDRHMIRMRQLIDIVDQLK | | |
| | | K81D, | NYVNDLVPEFLPAPEDVETGCEWSA | | |
| | | R114Q, | KSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | P107A, | MRSSPGNMERIVICLMVIFLGTLVH | 1.763253985 | 2597 |
| 9419 | 3610 | R114Q, | KSSSQGQDRHMIRMRQLIDIVDQLK | | |
| | | R115D | NYVNDLVPEFLPAPEDVETNCEWSA | | |
| | | | FSCFQKAQLKSANTGNNERIINVSI | | |
| | | | KKLKRKAPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | Q41D, | MRSSPGNMERIVICLMVIFLGTLVH | 1.763649727 | 2598 |
| 12773 | 0910 | I45T, | KSSSQGQDRHMIRMRDLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38A, | MRSSPGNMERIVICLMVIFLGTLVH | 1.772571 | 2599 |
| 10659 | 3610 | P107V, | KSSSQGQDRHMIAMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37G, | MRSSPGNMERIVICLMVIFLGTLVH | 1.783864414 | 2600 |
| 19043 | 5474 | N70G, | KSSSQGQDRHMGRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.784483903 | 2601 |
| 20255 | 5474 | K81D, | KSSSQGQDRHMIRMRQLIDIVDQLK | | |
| | | K104P, | NYVNDLVPEFLPAPEDVETGCEWSA | | |
| | | R114Q, | FSCFQDAQLKSANTGNNERIINVSI | | |
| | | R115D | KKLPRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.784954835 | 2602 |
| 18818 | 5707 | V98E, | KSSSQGQDRHMIRMRQLIDIVDQLK | | |
| | | P107V, | NYVNDLVPEFLPAPEDVETNCEWSA | | |
| | | P108K | FSCFQDAQLKSANTGNNERIINESI | | |
| | | | KKLKRKVKSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105Y, | MRSSPGNMERIVICLMVIFLGTLVH | 1.786129956 | 2603 |
| 9438 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKYKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | D44F, | MRSSPGNMERIVICLMVIFLGTLVH | 1.788776762 | 2604 |
| 19167 | 5474 | N70G, | KSSSQGQDRHMIRMRQLIFIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | D44F, | MRSSPGNMERIVICLMVIFLGTLVH | 1.7905758 | 2605 |
| 17664 | 5707 | K81D, | KSSSQGQDRHMIRMRQLIFIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38Q, | MRSSPGNMERIVICLMVIFLGTLVH | 1.807290972 | 2606 |
| 17566 | 5707 | K81D, | KSSSQGQDRHMIQMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.816652324 | 2607 |
| 14124 | 6318 | K81D, | KSSSQGQDRFMIRMRQLIDIVDQLK | | |
| | | V98H | NYVNDLVPEFLPAPEDVETNCEWSA | | |
| | | | FSCFQDAQLKSANTGNNERIINHSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34Y, | MRSSPGNMERIVICLMVIFLGTLVH | 1.821879136 | 2608 |
| 12881 | 0910 | I45T, | KSSSQGQDYHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.828017878 | 2609 |
| 18366 | 5707 | Q83F, | KSSSQGQDRHMIRMRQLIDIVDQLK | | |
| | | V98E, | NYVNDLVPEFLPAPEDVETNCEWSA | | |
| | | P107V | FSCFQDAFLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.834490433 | 2610 |
| 14743 | 6018 | L49F, | KSSSQGQDRFMIRMRQLIDIVDQFK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.83490278 | 2611 |
| 18649 | 5707 | V98Y, | KSSSQGQDRHMIRMRQLIDIVDQLK | | |
| | | P107V | NYVNDLVPEFLPAPEDVETNCEWSA | | |
| | | | FSCFQDAQLKSANTGNNERIINYSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34H, | MRSSPGNMERIVICLMVIFLGTLVH | 1.841821826 | 2612 |
| 12893 | 0910 | I45T, | KSSSQGQDHHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34D, | MRSSPGNMERIVICLMVIFLGTLVH | 1.844032452 | 2613 |
| 18986 | 5474 | N70G, | KSSSQGQDDHMIRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.846762744 | 2614 |
| 18775 | 5707 | V98E, | KSSSQGQDRHMIRMRQLIDIVDQLK | | |
| | | R105Y, | NYVNDLVPEFLPAPEDVETNCEWSA | | |
| | | P107V | FSCFQDAQLKSANTGNNERIINESI | | |
| | | | KKLKYKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.848014472 | 2615 |
| 19819 | 5474 | Q80H, | KSSSQGQDRHMIRMRQLIDIVDQLK | | |
| | | K81D, | NYVNDLVPEFLPAPEDVETGCEWSA | | |
| | | R114Q, | FSCFHDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.848667405 | 2616 |
| 13159 | 6318 | D44P, | KSSSQGQDRFMIRMRQLIPIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44G, | MRSSPGNMERIVICLMVIFLGTLVH | 1.852204231 | 2617 |
| 17665 | 5707 | K81D, | KSSSQGQDRHMIRMRQLIGIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34A, | MRSSPGNMERIVICLMVIFLGTLVH | 1.852558397 | 2618 |
| 12898 | 0910 | I45T, | KSSSQGQDAHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.853716812 | 2619 |
| 19913 | 5474 | K81D, | KSSSQGQDRHMIRMRQLIDIVDQLK | | |
| | | K85P, | NYVNDLVPEFLPAPEDVETGCEWSA | | |
| | | R114Q, | FSCFQDAQLPSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.866530475 | 2620 |
| 11671 | 0910 | K81D, | KSSSQGQDRHMIRMRQLIDTVDQLK | | |
| | | V98E, | NYVNDLVPEFLPAPEDVETNCEWSA | | |
| | | K102L | FSCFQDAQLKSANTGNNERIINESI | | |
| | | | KLLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.869008011 | 2621 |
| 11602 | 0910 | K81D, | KSSSQGQDRHMIRMRQLIDTVDQLK | | |
| | | V98E, | NYVNDLVPEFLPAPEDVETNCEWSA | | |
| | | K106G | FSCFQDAQLKSANTGNNERIINESI | | |
| | | | KKLKRGPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.876258345 | 2622 |
| 18663 | 5707 | V98E, | KSSSQGQDRHMIRMRQLIDIVDQLK | | |
| | | S99R, | NYVNDLVPEFLPAPEDVETNCEWSA | | |
| | | P107V | FSCFQDAQLKSANTGNNERIINERI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.888853469 | 2623 |
| 11569 | 0910 | K81D, | KSSSQGQDRHMIRMRQLIDTVDQLK | | |
| | | V98E, | NYVNDLVPEFLPAPEDVETNCEWSA | | |
| | | P108E | FSCFQDAQLKSANTGNNERIINESI | | |
| | | | KKLKRKPESTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44T, | MRSSPGNMERIVICLMVIFLGTLVH | 1.904052184 | 2624 |
| 12706 | 0910 | I45T, | KSSSQGQDRHMIRMRQLITTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.916642621 | 2625 |
| 11554 | 0910 | K81D, | KSSSQGQDRHMIRMRQLIDTVDQLK | | |
| | | V98E, | NYVNDLVPEFLPAPEDVETNCEWSA | | |
| | | P108Y | FSCFQDAQLKSANTGNNERIINESI | | |
| | | | KKLKRKPYSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44Y, | MRSSPGNMERIVICLMVIFLGTLVH | 1.917438735 | 2626 |
| 17679 | 5707 | K81D, | KSSSQGQDRHMIRMRQLIYIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37F, | MRSSPGNMERIVICLMVIFLGTLVH | 1.928208091 | 2627 |
| 12843 | 0910 | I45T, | KSSSQGQDRHMFRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | V98E, | MRSSPGNMERIVICLMVIFLGTLVH | 1.929634795 | 2628 |
| 5823 | 5707 | P107V | KSSSQGQDRHMIRMRQLIDIVDQLK | | |
| | | | NYVNDLVPEFLPAPEDVETNCEWSA | | |
| | | | FSCFQKAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.941003163 | 2629 |
| 14549 | 6018 | R38K, | KSSSQGQDRFMIKMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 1.94492025 | 2630 |
| 12043 | 0910 | K81R, | KSSSQGQDRHMIRMRQLIDTVDQLK | | |
| | | V98E | NYVNDLVPEFLPAPEDVETNCEWSA | | |
| | | | FSCFQRAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | G90Q, | MRSSPGNMERIVICLMVIFLGTLVH | 1.945741822 | 2631 |
| 9713 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTQNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | D44A, | MRSSPGNMERIVICLMVIFLGTLVH | 1.966844929 | 2632 |
| 12720 | 0910 | I45T, | KSSSQGQDRHMIRMRQLIATVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 1.967064093 | 2633 |
| 18766 | 5707 | V98E, | KSSSQGQDRHMIRMRQLIDIVDQLK | | |
| | | R105L, | NYVNDLVPEFLPAPEDVETNCEWSA | | |
| | | P107V | FSCFQDAQLKSANTGNNERIINESI | | |
| | | | KKLKLKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 1.973653405 | 2634 |
| 14621 | 6018 | L42K, | KSSSQGQDRFMIRMRQKIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | R38M, | MRSSPGNMERIVICLMVIFLGTLVH | 1.974843424 | 2635 |
| 17563 | 5707 | K81D, | KSSSQGQDRHMIMMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105L, | MRSSPGNMERIVICLMVIFLGTLVH | 1.977922998 | 2636 |
| 9447 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKLKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81R, | MRSSPGNMERIVICLMVIFLGTLVH | 1.985918259 | 2637 |
| 18339 | 5707 | V98E, | KSSSQGQDRHMIRMRQLIDIVDQLK | | |
| | | P107V | NYVNDLVPEFLPAPEDVETNCEWSA | | |
| | | | FSCFQRAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 1.998621431 | 2638 |
| 19895 | 5474 | K81D, | KSSSQGQDRHMIRMRQLIDIVDQLK | | |
| | | L84P, | NYVNDLVPEFLPAPEDVETGCEWSA | | |
| | | R114Q, | FSCFQDAQPKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38W, | MRSSPGNMERIVICLMVIFLGTLVH | 2.00707852 | 2639 |
| 17570 | 5707 | K81D, | KSSSQGQDRHMIWMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37G, | MRSSPGNMERIVICLMVIFLGTLVH | 2.007511318 | 2640 |
| 10673 | 3610 | P107V, | KSSSQGQDRHMGRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.018535778 | 2641 |
| 20162 | 5474 | K81D, | KSSSQGQDRHMIRMRQLIDIVDQLK | | |
| | | S99K, | NYVNDLVPEFLPAPEDVETGCEWSA | | |
| | | R114Q, | FSCFQDAQLKSANTGNNERIINVKI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37R, | MRSSPGNMERIVICLMVIFLGTLVH | 2.021430425 | 2642 |
| 19051 | 5474 | N70G, | KSSSQGQDRHMRRMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.024439352 | 2643 |
| 20270 | 5474 | K81D, | KSSSQGQDRHMIRMRQLIDIVDQLK | | |
| | | R105K, | NYVNDLVPEFLPAPEDVETGCEWSA | | |
| | | R114Q, | FSCFQDAQLKSANTGNNERIINVSI | | |
| | | R115D | KKLKKKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37H, | MRSSPGNMERIVICLMVIFLGTLVH | 2.031647072 | 2644 |
| 12841 | 0910 | I45T, | KSSSQGQDRHMHRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.040750453 | 2645 |
| 14288 | 6318 | K81D, | KSSSQGQDRFMIRMRQLIDIVDQLK | | |
| | | V98E, | NYVNDLVPEFLPAPEDVETNCEWSA | | |
| | | P107K | FSCFQDAQLKSANTGNNERIINESI | | |
| | | | KKLKRKKPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.049793877 | 2646 |
| 7545 | 4177 | N70G, | KSSSQGQDRIMIRMRQLIDIVDQLK | | |
| | | G90Q, | NYVNDLVPEFLPAPEDVETGCEWSA | | |
| | | T110D, | FSCFQKAQLKSANTQNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.053724225 | 2647 |
| 13182 | 6318 | I45W, | KSSSQGQDRFMIRMRQLIDWVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.059603982 | 2648 |
| 15597 | 6018 | K81D, | KSSSQGQDRFMIRMRQLIDIVDQLK | | |
| | | 196P, | NYVNDLVPEFLPAPEDVETNCEWSA | | |
| | | V98E, | FSCFQDAQLKSANTGNNERIPNESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.069162639 | 2649 |
| 13050 | 6318 | R38M, | KSSSQGQDRFMIMMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.07037494 | 2650 |
| 20101 | 5474 | K81D, | KSSSQGQDRHMIRMRQLIDIVDQLK | | |
| | | 196A, | NYVNDLVPEFLPAPEDVETGCEWSA | | |
| | | R114Q, | FSCFQDAQLKSANTGNNERIANVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.07118802 | 2651 |
| 7580 | 4177 | N70G, | KSSSQGQDRIMIRMRQLIDIVDQLK | | |
| | | N88R, | NYVNDLVPEFLPAPEDVETGCEWSA | | |
| | | T110D, | FSCFQKAQLKSARTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.073824213 | 2652 |
| 7140 | 4177 | N70G, | KSSSQGQDRIMIRMRQLIDIVDQLK | | |
| | | T110D, | NYVNDLVPEFLPAPEDVETGCEWSA | | |
| | | A112P, | FSCFQKAQLKSANTGNNERIINVSI | | |
| | | G113H | KKLKRKPPSDNPHRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 2.074659355 | 2653 |
| 18763 | 5707 | V98E, | KSSSQGQDRHMIRMRQLIDIVDQLK | | |
| | | R105H, | NYVNDLVPEFLPAPEDVETNCEWSA | | |
| | | P107V | FSCFQDAQLKSANTGNNERIINESI | | |
| | | | KKLKHKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.080648353 | 2654 |
| 7172 | 4177 | N70G, | KSSSQGQDRIMIRMRQLIDIVDQLK | | |
| | | T110D, | NYVNDLVPEFLPAPEDVETGCEWSA | | |
| | | N111K, | FSCFQKAQLKSANTGNNERIINVSI | | |
| | | A112P | KKLKRKPPSDKPGRRQKHRLTCPSC | | |
| | | | DSYEKKPPKEFLERFKSLLQKMIHQ | | |
| | | | HLSSRTHGSEDS | | |
| IL-21_ | IL-21_ | I45W, | MRSSPGNMERIVICLMVIFLGTLVH | 2.081701336 | 2655 |
| 17695 | 5707 | K81D, | KSSSQGQDRHMIRMRQLIDWVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.082266996 | 2656 |
| 19856 | 5474 | K81D, | KSSSQGQDRHMIRMRQLIDIVDQLK | | |
| | | A82L, | NYVNDLVPEFLPAPEDVETGCEWSA | | |
| | | R114Q, | FSCFQDLQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.088418702 | 2657 |
| 20103 | 5474 | K81D, | KSSSQGQDRHMIRMRQLIDIVDQLK | | |
| | | 196E, | NYVNDLVPEFLPAPEDVETGCEWSA | | |
| | | R114Q, | FSCFQDAQLKSANTGNNERIENVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.09005915 | 2658 |
| 8501 | 4177 | I37P, | KSSSQGQDRIMPRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.09128725 | 2659 |
| 13738 | 6318 | F76T, | KSSSQGQDRFMIRMRQLIDIVDQLK | | |
| | | K81D, | NYVNDLVPEFLPAPEDVETNCEWSA | | |
| | | V98E | TSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.093814801 | 2660 |
| 7516 | 4177 | N70G, | KSSSQGQDRIMIRMRQLIDIVDQLK | | |
| | | N92G, | NYVNDLVPEFLPAPEDVETGCEWSA | | |
| | | T110D, | FSCFQKAQLKSANTGNGERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.096793399 | 2661 |
| 14293 | 6318 | K81D, | KSSSQGQDRFMIRMRQLIDIVDQLK | | |
| | | V98E, | NYVNDLVPEFLPAPEDVETNCEWSA | | |
| | | P107R | FSCFQDAQLKSANTGNNERIINESI | | |
| | | | KKLKRKRPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.099827872 | 2662 |
| 15239 | 6018 | F76R, | KSSSQGQDRFMIRMRQLIDIVDQLK | | |
| | | K81D, | NYVNDLVPEFLPAPEDVETNCEWSA | | |
| | | V98E, | RSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.103074525 | 2663 |
| 7223 | 4177 | N70G, | KSSSQGQDRIMIRMRQLIDIVDQLK | | |
| | | P108M, | NYVNDLVPEFLPAPEDVETGCEWSA | | |
| | | T110D, | FSCFQKAQLKSANTGNNERIINVSI | | |
| | | A112P | KKLKRKPMSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.103660646 | 2664 |
| 13048 | 6318 | R38K, | KSSSQGQDRFMIKMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.106163316 | 2665 |
| 8477 | 4177 | R38W, | KSSSQGQDRIMIWMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.106485886 | 2666 |
| 7183 | 4177 | N70G, | KSSSQGQDRIMIRMRQLIDIVDQLK | | |
| | | T110S, | NYVNDLVPEFLPAPEDVETGCEWSA | | |
| | | A112P | FSCFQKAQLKSANTGNNERIINVSI | | |
| | | | KKLKRKPPSSNPGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 2.107292106 | 2667 |
| 12642 | 0910 | Q48K, | KSSSQGQDRHMIRMRQLIDTVDKLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37R, | MRSSPGNMERIVICLMVIFLGTLVH | 2.108337686 | 2668 |
| 10665 | 3610 | P107V, | KSSSQGQDRHMRRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38K, | MRSSPGNMERIVICLMVIFLGTLVH | 2.109827531 | 2669 |
| 17561 | 5707 | K81D, | KSSSQGQDRHMIKMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.114136025 | 2670 |
| 7489 | 4177 | N70G, | KSSSQGQDRIMIRMRQLIDIVDQLK | | |
| | | E93S, | NYVNDLVPEFLPAPEDVETGCEWSA | | |
| | | T110D, | FSCFQKAQLKSANTGNNSRIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.124936797 | 2671 |
| 7005 | 4177 | N70G, | KSSSQGQDRIMIRMRQLIDIVDQLK | | |
| | | T110D, | NYVNDLVPEFLPAPEDVETGCEWSA | | |
| | | A112P, | FSCFQKAQLKSANTGNNERIINVSI | | |
| | | L120S | KKLKRKPPSDNPGRRQKHRSTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.125623049 | 2672 |
| 7488 | 4177 | N70G, | KSSSQGQDRIMIRMRQLIDIVDQLK | | |
| | | E93T, | NYVNDLVPEFLPAPEDVETGCEWSA | | |
| | | T110D, | FSCFQKAQLKSANTGNNTRIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.125774336 | 2673 |
| 8500 | 4177 | I37Q, | KSSSQGQDRIMQRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.12833226 | 2674 |
| 14253 | 6318 | K81D, | KSSSQGQDRFMIRMRQLIDIVDQLK | | |
| | | V98E, | NYVNDLVPEFLPAPEDVETNCEWSA | | |
| | | R105L | FSCFQDAQLKSANTGNNERIINESI | | |
| | | | KKLKLKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.12921114 | 2675 |
| 8166 | 4177 | L56F, | KSSSQGQDRIMIRMRQLIDIVDQLK | | |
| | | N70G, | NYVNDFVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.131265802 | 2676 |
| 7222 | 4177 | N70G, | KSSSQGQDRIMIRMRQLIDIVDQLK | | |
| | | P108N, | NYVNDLVPEFLPAPEDVETGCEWSA | | |
| | | T110D, | FSCFQKAQLKSANTGNNERIINVSI | | |
| | | A112P | KKLKRKPNSDNPGRRQKHRLTCPSC | | |
| | | | DSYEKKPPKEFLERFKSLLQKMIHQ | | |
| | | | HLSSRTHGSEDS | | |
| IL-21_ | IL-21_ | E65R, | MRSSPGNMERIVICLMVIFLGTLVH | 2.13367096 | 2677 |
| 16548 | 5845 | N70G, | KSSSQGQDRHMIRMRQLIDIVDQLK | | |
| | | K81D | NYVNDLVPEFLPAPRDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.134473328 | 2678 |
| 8100 | 4177 | E59V, | KSSSQGQDRIMIRMRQLIDIVDQLK | | |
| | | N70G, | NYVNDLVPVELPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | D44Q, | MRSSPGNMERIVICLMVIFLGTLVH | 2.134833056 | 2679 |
| 16170 | 5845 | N70G, | KSSSQGQDRHMIRMRQLIQIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.135970401 | 2680 |
| 17350 | 5845 | K81D, | KSSSQGQDRHMIRMRQLIDIVDQLK | | |
| | | T110H | NYVNDLVPEFLPAPEDVETGCEWSA | | |
| | | | FSCFQDAQLKSANTGNNERIINVSI | | |
| | | | KKLKRKPPSHNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.140647089 | 2681 |
| 7619 | 4177 | N70G, | KSSSQGQDRIMIRMRQLIDIVDQLK | | |
| | | S86M, | NYVNDLVPEFLPAPEDVETGCEWSA | | |
| | | T110D, | FSCFQKAQLKMANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.145496152 | 2682 |
| 13041 | 6318 | R38A, | KSSSQGQDRFMIAMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | E59Y, | MRSSPGNMERIVICLMVIFLGTLVH | 2.145911428 | 2683 |
| 16445 | 5845 | N70G, | KSSSQGQDRHMIRMRQLIDIVDQLK | | |
| | | K81D | NYVNDLVPYFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44S, | MRSSPGNMERIVICLMVIFLGTLVH | 2.146037087 | 2684 |
| 12707 | 0910 | I45T, | KSSSQGQDRHMIRMRQLISTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.146632486 | 2685 |
| 7187 | 4177 | N70G, | KSSSQGQDRIMIRMRQLIDIVDQLK | | |
| | | T110N, | NYVNDLVPEFLPAPEDVETGCEWSA | | |
| | | A112P | FSCFQKAQLKSANTGNNERIINVSI | | |
| | | | KKLKRKPPSNNPGRRQKHRLTCPSC | | |
| | | | DSYEKKPPKEFLERFKSLLQKMIHQ | | |
| | | | HLSSRTHGSEDS | | |
| IL-21_ | IL-21_ | D55Y, | MRSSPGNMERIVICLMVIFLGTLVH | 2.148614987 | 2686 |
| 16373 | 5845 | N70G, | KSSSQGQDRHMIRMRQLIDIVDQLK | | |
| | | K81D | NYVNYLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.148852592 | 2687 |
| 15604 | 6018 | K81D, | KSSSQGQDRFMIRMRQLIDIVDQLK | | |
| | | 196Y, | NYVNDLVPEFLPAPEDVETNCEWSA | | |
| | | V98E, | FSCFQDAQLKSANTGNNERIYNESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.150582557 | 2688 |
| 6576 | 4177 | N70G, | KSSSQGQDRIMIRMRQLIDIVDQLK | | |
| | | T110D, | NYVNDLVPEFLPAPEDVETGCEWSA | | |
| | | A112P, | FSCFQKAQLKSANTGNNERIINVSI | | |
| | | L144M | KKLKRKPPSDNPGRRQKHRLTCPSC | | |
| | | | DSYEKKPPKEFLERFKSLMQKMIHQ | | |
| | | | HLSSRTHGSEDS | | |
| IL-21_ | IL-21_ | D33W, | MRSSPGNMERIVICLMVIFLGTLVH | 2.152164842 | 2689 |
| 15979 | 5845 | N70G, | KSSSQGQWRHMIRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.152273267 | 2690 |
| 7686 | 4177 | N70G, | KSSSQGQDRIMIRMRQLIDIVDQLK | | |
| | | A82T, | NYVNDLVPEFLPAPEDVETGCEWSA | | |
| | | T110D, | FSCFQKTQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.154181035 | 2691 |
| 16869 | 5845 | K81D, | KSSSQGQDRHMIRMRQLIDIVDQLK | | |
| | | Q83K | NYVNDLVPEFLPAPEDVETGCEWSA | | |
| | | | FSCFQDAKLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.154817069 | 2692 |
| 7736 | 4177 | N70G, | KSSSQGQDRIMIRMRQLIDIVDQLK | | |
| | | Q80A, | NYVNDLVPEFLPAPEDVETGCEWSA | | |
| | | T110D, | FSCFAKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.157664249 | 2693 |
| 17057 | 5845 | K81D, | KSSSQGQDRHMIRMRQLIDIVDQLK | | |
| | | E93V | NYVNDLVPEFLPAPEDVETGCEWSA | | |
| | | | FSCFQDAQLKSANTGNNVRIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.16174098 | 2694 |
| 17227 | 5845 | K81D, | KSSSQGQDRHMIRMRQLIDIVDQLK | | |
| | | L103I | NYVNDLVPEFLPAPEDVETGCEWSA | | |
| | | | FSCFQDAQLKSANTGNNERIINVSI | | |
| | | | KKIKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Y52H, | MRSSPGNMERIVICLMVIFLGTLVH | 2.165790853 | 2695 |
| 16307 | 5845 | N70G, | KSSSQGQDRHMIRMRQLIDIVDQLK | | |
| | | K81D | NHVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.166075096 | 2696 |
| 16993 | 5845 | K81D, | KSSSQGQDRHMIRMRQLIDIVDQLK | | |
| | | G90I | NYVNDLVPEFLPAPEDVETGCEWSA | | |
| | | | FSCFQDAQLKSANTINNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.166954881 | 2697 |
| 13111 | 6318 | Q41W, | KSSSQGQDRFMIRMRWLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.167668321 | 2698 |
| 15757 | 6018 | K81D, | KSSSQGQDRFMIRMRQLIDIVDQLK | | |
| | | V98E, | NYVNDLVPEFLPAPEDVETNCEWSA | | |
| | | R105M, | FSCFQDAQLKSANTGNNERIINESI | | |
| | | T110D | KKLKMKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.170126289 | 2699 |
| 14167 | 6318 | K81D, | KSSSQGQDRFMIRMRQLIDIVDQLK | | |
| | | V98E, | NYVNDLVPEFLPAPEDVETNCEWSA | | |
| | | I100R | FSCFQDAQLKSANTGNNERIINESR | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D55M, | MRSSPGNMERIVICLMVIFLGTLVH | 2.170458815 | 2700 |
| 16364 | 5845 | N70G, | KSSSQGQDRHMIRMRQLIDIVDQLK | | |
| | | K81D | NYVNMLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D66K, | MRSSPGNMERIVICLMVIFLGTLVH | 2.17573168 | 2701 |
| 16560 | 5845 | N70G, | KSSSQGQDRHMIRMRQLIDIVDQLK | | |
| | | K81D | NYVNDLVPEFLPAPEKVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.176321443 | 2702 |
| 16821 | 5845 | Q80S, | KSSSQGQDRHMIRMRQLIDIVDQLK | | |
| | | K81D | NYVNDLVPEFLPAPEDVETGCEWSA | | |
| | | | FSCFSDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.17705247 | 2703 |
| 16944 | 5845 | K81D, | KSSSQGQDRHMIRMRQLIDIVDQLK | | |
| | | A87P | NYVNDLVPEFLPAPEDVETGCEWSA | | |
| | | | FSCFQDAQLKSPNTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.180018661 | 2704 |
| 7150 | 4177 | N70G, | KSSSQGQDRIMIRMRQLIDIVDQLK | | |
| | | T110D, | NYVNDLVPEFLPAPEDVETGCEWSA | | |
| | | A112R | FSCFQKAQLKSANTGNNERIINVSI | | |
| | | | KKLKRKPPSDNRGRRQKHRLTCPSC | | |
| | | | DSYEKKPPKEFLERFKSLLQKMIHQ | | |
| | | | HLSSRTHGSEDS | | |
| IL-21_ | IL-21_ | M36R, | MRSSPGNMERIVICLMVIFLGTLVH | 2.180793616 | 2705 |
| 16027 | 5845 | N70G, | KSSSQGQDRHRIRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.181254 | 2706 |
| 7503 | 4177 | N70G, | KSSSQGQDRIMIRMRQLIDIVDQLK | | |
| | | N92Y, | NYVNDLVPEFLPAPEDVETGCEWSA | | |
| | | T110D, | FSCFQKAQLKSANTGNYERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.181308395 | 2707 |
| 14222 | 6318 | K81D, | KSSSQGQDRFMIRMRQLIDIVDQLK | | |
| | | V98E, | NYVNDLVPEFLPAPEDVETNCEWSA | | |
| | | L103S | FSCFQDAQLKSANTGNNERIINESI | | |
| | | | KKSKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.182220081 | 2708 |
| 7784 | 4177 | N70G, | KSSSQGQDRIMIRMRQLIDIVDQLK | | |
| | | S77K, | NYVNDLVPEFLPAPEDVETGCEWSA | | |
| | | T110D, | FKCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | Q80A, | MRSSPGNMERIVICLMVIFLGTLVH | 2.183142292 | 2709 |
| 9903 | 3610 | P107V, | KSSSQGQDRHMIRMRQLIDIVDQLK | | |
| | | R114Q, | NYVNDLVPEFLPAPEDVETNCEWSA | | |
| | | R115D | FSCFAKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | E68G, | MRSSPGNMERIVICLMVIFLGTLVH | 2.186384581 | 2710 |
| 16593 | 5845 | N70G, | KSSSQGQDRHMIRMRQLIDIVDQLK | | |
| | | K81D | NYVNDLVPEFLPAPEDVGTGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.186975479 | 2711 |
| 17318 | 5845 | K81D, | KSSSQGQDRHMIRMRQLIDIVDQLK | | |
| | | P108L | NYVNDLVPEFLPAPEDVETGCEWSA | | |
| | | | FSCFQDAQLKSANTGNNERIINVSI | | |
| | | | KKLKRKPLSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.188118585 | 2712 |
| 16677 | 5845 | E72T, | KSSSQGQDRHMIRMRQLIDIVDQLK | | |
| | | K81D | NYVNDLVPEFLPAPEDVETGCTWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.188121687 | 2713 |
| 7132 | 4177 | N70G, | KSSSQGQDRIMIRMRQLIDIVDQLK | | |
| | | T110D, | NYVNDLVPEFLPAPEDVETGCEWSA | | |
| | | A112P, | FSCFQKAQLKSANTGNNERIINVSI | | |
| | | G113R | KKLKRKPPSDNPRRRQKHRLTCPSC | | |
| | | | DSYEKKPPKEFLERFKSLLQKMIHQ | | |
| | | | HLSSRTHGSEDS | | |
| IL-21_ | IL-21_ | I37K, | MRSSPGNMERIVICLMVIFLGTLVH | 2.19187472 | 2714 |
| 10671 | 3610 | P107V, | KSSSQGQDRHMKRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.19566728 | 2715 |
| 17280 | 5845 | K81D, | KSSSQGQDRHMIRMRQLIDIVDQLK | | |
| | | K106H | NYVNDLVPEFLPAPEDVETGCEWSA | | |
| | | | FSCFQDAQLKSANTGNNERIINVSI | | |
| | | | KKLKRHPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D33V, | MRSSPGNMERIVICLMVIFLGTLVH | 2.196212518 | 2716 |
| 15978 | 5845 | N70G, | KSSSQGQVRHMIRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D33T, | MRSSPGNMERIVICLMVIFLGTLVH | 2.19671623 | 2717 |
| 15977 | 5845 | N70G, | KSSSQGQTRHMIRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.199634927 | 2718 |
| 17327 | 5845 | K81D, | KSSSQGQDRHMIRMRQLIDIVDQLK | | |
| | | P108Y | NYVNDLVPEFLPAPEDVETGCEWSA | | |
| | | | FSCFQDAQLKSANTGNNERIINVSI | | |
| | | | KKLKRKPYSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 2.199739522 | 2719 |
| 11555 | 0910 | K81D, | KSSSQGQDRHMIRMRQLIDTVDQLK | | |
| | | V98E, | NYVNDLVPEFLPAPEDVETNCEWSA | | |
| | | P108W | FSCFQDAQLKSANTGNNERIINESI | | |
| | | | KKLKRKPWSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 2.203468362 | 2720 |
| 18643 | 5707 | V98P, | KSSSQGQDRHMIRMRQLIDIVDQLK | | |
| | | P107V | NYVNDLVPEFLPAPEDVETNCEWSA | | |
| | | | FSCFQDAQLKSANTGNNERIINPSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.203715507 | 2721 |
| 17055 | 5845 | K81D, | KSSSQGQDRHMIRMRQLIDIVDQLK | | |
| | | E93S | NYVNDLVPEFLPAPEDVETGCEWSA | | |
| | | | FSCFQDAQLKSANTGNNSRIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 2.204284899 | 2722 |
| 18722 | 5707 | V98E, | KSSSQGQDRHMIRMRQLIDIVDQLK | | |
| | | L103A, | NYVNDLVPEFLPAPEDVETNCEWSA | | |
| | | P107V | FSCFQDAQLKSANTGNNERIINESI | | |
| | | | KKAKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.206065275 | 2723 |
| 8214 | 4177 | V53M, | KSSSQGQDRIMIRMRQLIDIVDQLK | | |
| | | N70G, | NYMNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 2.207023856 | 2724 |
| 18801 | 5707 | V98E, | KSSSQGQDRHMIRMRQLIDIVDQLK | | |
| | | P107K | NYVNDLVPEFLPAPEDVETNCEWSA | | |
| | | | FSCFQDAQLKSANTGNNERIINESI | | |
| | | | KKLKRKKPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I43Y, | MRSSPGNMERIVICLMVIFLGTLVH | 2.207419577 | 2725 |
| 16158 | 5845 | N70G, | KSSSQGQDRHMIRMRQLYDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.21417666 | 2726 |
| 19866 | 5474 | K81D, | KSSSQGQDRHMIRMRQLIDIVDQLK | | |
| | | A82Y, | NYVNDLVPEFLPAPEDVETGCEWSA | | |
| | | R114Q, | FSCFQDYQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.216214166 | 2727 |
| 17040 | 5845 | K81D, | KSSSQGQDRHMIRMRQLIDIVDQLK | | |
| | | N92W | NYVNDLVPEFLPAPEDVETGCEWSA | | |
| | | | FSCFQDAQLKSANTGNWERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.217235225 | 2728 |
| 8374 | 4177 | D44Q, | KSSSQGQDRIMIRMRQLIQIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.222310266 | 2729 |
| 7726 | 4177 | N70G, | KSSSQGQDRIMIRMRQLIDIVDQLK | | |
| | | Q80N, | NYVNDLVPEFLPAPEDVETGCEWSA | | |
| | | T110D, | FSCENKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 2.225320276 | 2730 |
| 11677 | 0910 | K81D, | KSSSQGQDRHMIRMRQLIDTVDQLK | | |
| | | V98E, | NYVNDLVPEFLPAPEDVETNCEWSA | | |
| | | K102D | FSCFQDAQLKSANTGNNERIINESI | | |
| | | | KDLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.226508641 | 2731 |
| 16749 | 5845 | F76T, | KSSSQGQDRHMIRMRQLIDIVDQLK | | |
| | | K81D | NYVNDLVPEFLPAPEDVETGCEWSA | | |
| | | | TSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.227481614 | 2732 |
| 16839 | 5845 | K81S | KSSSQGQDRHMIRMRQLIDIVDQLK | | |
| | | | NYVNDLVPEFLPAPEDVETGCEWSA | | |
| | | | FSCFQSAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.227514134 | 2733 |
| 7778 | 4177 | N70G, | KSSSQGQDRIMIRMRQLIDIVDQLK | | |
| | | S77R, | NYVNDLVPEFLPAPEDVETGCEWSA | | |
| | | T110D, | FRCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.232032473 | 2734 |
| 7246 | 4177 | N70G, | KSSSQGQDRIMIRMRQLIDIVDQLK | | |
| | | P107G, | NYVNDLVPEFLPAPEDVETGCEWSA | | |
| | | T110D, | FSCFQKAQLKSANTGNNERIINVSI | | |
| | | A112P | KKLKRKGPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 2.239973514 | 2735 |
| 18767 | 5707 | V98E, | KSSSQGQDRHMIRMRQLIDIVDQLK | | |
| | | R105M, | NYVNDLVPEFLPAPEDVETNCEWSA | | |
| | | P107V | FSCFQDAQLKSANTGNNERIINESI | | |
| | | | KKLKMKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.240313095 | 2736 |
| 8370 | 4177 | D44V, | KSSSQGQDRIMIRMRQLIVIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.247636884 | 2737 |
| 7775 | 4177 | N70G, | KSSSQGQDRIMIRMRQLIDIVDQLK | | |
| | | S77W, | NYVNDLVPEFLPAPEDVETGCEWSA | | |
| | | T110D, | FWCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.247907431 | 2738 |
| 7390 | 4177 | N70G, | KSSSQGQDRIMIRMRQLIDIVDQLK | | |
| | | S99G, | NYVNDLVPEFLPAPEDVETGCEWSA | | |
| | | T110D, | FSCFQKAQLKSANTGNNERIINVGI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.24868668 | 2739 |
| 20186 | 5474 | K81D, | KSSSQGQDRHMIRMRQLIDIVDQLK | | |
| | | I100S, | NYVNDLVPEFLPAPEDVETGCEWSA | | |
| | | R114Q, | FSCFQDAQLKSANTGNNERIINVSS | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 2.24889862 | 2740 |
| 11663 | 0910 | K81D, | KSSSQGQDRHMIRMRQLIDTVDQLK | | |
| | | V98E, | NYVNDLVPEFLPAPEDVETNCEWSA | | |
| | | K102V | FSCFQDAQLKSANTGNNERIINESI | | |
| | | | KVLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.249882244 | 2741 |
| 16964 | 5845 | K81D, | KSSSQGQDRHMIRMRQLIDIVDQLK | | |
| | | N88R | NYVNDLVPEFLPAPEDVETGCEWSA | | |
| | | | FSCFQDAQLKSARTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.252733497 | 2742 |
| 8509 | 4177 | I37E, | KSSSQGQDRIMERMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.257488828 | 2743 |
| 16706 | 5845 | S74K, | KSSSQGQDRHMIRMRQLIDIVDQLK | | |
| | | K81D | NYVNDLVPEFLPAPEDVETGCEWKA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.258091769 | 2744 |
| 7409 | 4177 | N70G, | KSSSQGQDRIMIRMRQLIDIVDQLK | | |
| | | V98F, | NYVNDLVPEFLPAPEDVETGCEWSA | | |
| | | T110D, | FSCFQKAQLKSANTGNNERIINESI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.25934197 | 2745 |
| 17047 | 5845 | K81D, | KSSSQGQDRHMIRMRQLIDIVDQLK | | |
| | | E93I | NYVNDLVPEFLPAPEDVETGCEWSA | | |
| | | | FSCFQDAQLKSANTGNNIRIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.261753111 | 2746 |
| 16867 | 5845 | K81D, | KSSSQGQDRHMIRMRQLIDIVDQLK | | |
| | | Q83H | NYVNDLVPEFLPAPEDVETGCEWSA | | |
| | | | FSCFQDAHLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.261999435 | 2747 |
| 14095 | 6318 | K81D, | KSSSQGQDRFMIRMRQLIDIVDQLK | | |
| | | 196Q, | NYVNDLVPEFLPAPEDVETNCEWSA | | |
| | | V98E | FSCFQDAQLKSANTGNNERIQNESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.264466711 | 2748 |
| 15589 | 6018 | K81D, | KSSSQGQDRFMIRMRQLIDIVDQLK | | |
| | | 196E, | NYVNDLVPEFLPAPEDVETNCEWSA | | |
| | | V98E, | FSCFQDAQLKSANTGNNERIENESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.265171147 | 2749 |
| 17082 | 5845 | K81D, | KSSSQGQDRHMIRMRQLIDIVDQLK | | |
| | | 195G | NYVNDLVPEFLPAPEDVETGCEWSA | | |
| | | | FSCFQDAQLKSANTGNNERGINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.275441678 | 2750 |
| 8495 | 4177 | I37W, | KSSSQGQDRIMWRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 2.282310747 | 2751 |
| 18637 | 5707 | V98H, | KSSSQGQDRHMIRMRQLIDIVDQLK | | |
| | | P107V | NYVNDLVPEFLPAPEDVETNCEWSA | | |
| | | | FSCFQDAQLKSANTGNNERIINHSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | V67L, | MRSSPGNMERIVICLMVIFLGTLVH | 2.287553091 | 2752 |
| 16580 | 5845 | N70G, | KSSSQGQDRHMIRMRQLIDIVDQLK | | |
| | | K81D | NYVNDLVPEFLPAPEDLETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.290443184 | 2753 |
| 16688 | 5845 | W73K, | KSSSQGQDRHMIRMRQLIDIVDQLK | | |
| | | K81D | NYVNDLVPEFLPAPEDVETGCEKSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I43F, | MRSSPGNMERIVICLMVIFLGTLVH | 2.290724331 | 2754 |
| 16144 | 5845 | N70G, | KSSSQGQDRHMIRMRQLFDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.297622558 | 2755 |
| 17298 | 5845 | K81D, | KSSSQGQDRHMIRMRQLIDIVDQLK | | |
| | | P107H | NYVNDLVPEFLPAPEDVETGCEWSA | | |
| | | | FSCFQDAQLKSANTGNNERIINVSI | | |
| | | | KKLKRKHPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70P, | MRSSPGNMERIVICLMVIFLGTLVH | 2.297793725 | 2756 |
| 16636 | 5845 | K81D | KSSSQGQDRHMIRMRQLIDIVDQLK | | |
| | | | NYVNDLVPEFLPAPEDVETPCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.299317227 | 2757 |
| 16834 | 5845 | K81M | KSSSQGQDRHMIRMRQLIDIVDQLK | | |
| | | | NYVNDLVPEFLPAPEDVETGCEWSA | | |
| | | | FSCFQMAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | L56S, | MRSSPGNMERIVICLMVIFLGTLVH | 2.299498227 | 2758 |
| 19394 | 5474 | N70G, | KSSSQGQDRHMIRMRQLIDIVDQLK | | |
| | | K81D, | NYVNDSVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | V98K, | MRSSPGNMERIVICLMVIFLGTLVH | 2.300233633 | 2759 |
| 9574 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINKSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.305812291 | 2760 |
| 17059 | 5845 | K81D, | KSSSQGQDRHMIRMRQLIDIVDQLK | | |
| | | E93Y | NYVNDLVPEFLPAPEDVETGCEWSA | | |
| | | | FSCFQDAQLKSANTGNNYRIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.306153022 | 2761 |
| 8498 | 4177 | I37S, | KSSSQGQDRIMSRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.306508948 | 2762 |
| 8510 | 4177 | I37D, | KSSSQGQDRIMDRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.306872497 | 2763 |
| 16810 | 5845 | Q80E, | KSSSQGQDRHMIRMRQLIDIVDQLK | | |
| | | K81D | NYVNDLVPEFLPAPEDVETGCEWSA | | |
| | | | FSCFEDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.307418786 | 2764 |
| 14570 | 6018 | M39P, | KSSSQGQDRFMIRPRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.310064986 | 2765 |
| 17056 | 5845 | K81D, | KSSSQGQDRHMIRMRQLIDIVDQLK | | |
| | | E93T | NYVNDLVPEFLPAPEDVETGCEWSA | | |
| | | | FSCFQDAQLKSANTGNNTRIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105F, | MRSSPGNMERIVICLMVIFLGTLVH | 2.315304129 | 2766 |
| 9452 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKFKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.317688487 | 2767 |
| 8385 | 4177 | D44A, | KSSSQGQDRIMIRMRQLIAIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45W, | MRSSPGNMERIVICLMVIFLGTLVH | 2.321466573 | 2768 |
| 19199 | 5474 | N70G, | KSSSQGQDRHMIRMRQLIDWVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.33530255 | 2769 |
| 7233 | 4177 | N70G, | KSSSQGQDRIMIRMRQLIDIVDQLK | | |
| | | P107Y, | NYVNDLVPEFLPAPEDVETGCEWSA | | |
| | | T110D, | FSCFQKAQLKSANTGNNERIINVSI | | |
| | | A112P | KKLKRKYPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.342215031 | 2770 |
| 17387 | 5845 | K81D, | KSSSQGQDRHMIRMRQLIDIVDQLK | | |
| | | A112K | NYVNDLVPEFLPAPEDVETGCEWSA | | |
| | | | FSCFQDAQLKSANTGNNERIINVSI | | |
| | | | KKLKRKPPSTNKGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.342806878 | 2771 |
| 7730 | 4177 | N70G, | KSSSQGQDRIMIRMRQLIDIVDQLK | | |
| | | Q80I, | NYVNDLVPEFLPAPEDVETGCEWSA | | |
| | | T110D, | FSCFIKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45S, | MRSSPGNMERIVICLMVIFLGTLVH | 2.359326608 | 2772 |
| 16190 | 5845 | N70G, | KSSSQGQDRHMIRMRQLIDSVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Q48K, | MRSSPGNMERIVICLMVIFLGTLVH | 2.364697651 | 2773 |
| 16237 | 5845 | N70G, | KSSSQGQDRHMIRMRQLIDIVDKLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.367834744 | 2774 |
| 16994 | 5845 | K81D, | KSSSQGQDRHMIRMRQLIDIVDQLK | | |
| | | G90K | NYVNDLVPEFLPAPEDVETGCEWSA | | |
| | | | FSCFQDAQLKSANTKNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44S, | MRSSPGNMERIVICLMVIFLGTLVH | 2.368609322 | 2775 |
| 16172 | 5845 | N70G, | KSSSQGQDRHMIRMRQLISIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.369055148 | 2776 |
| 16818 | 5845 | Q80N, | KSSSQGQDRHMIRMRQLIDIVDQLK | | |
| | | K81D | NYVNDLVPEFLPAPEDVETGCEWSA | | |
| | | | FSCENDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 2.36927041 | 2777 |
| 18765 | 5707 | V98E, | KSSSQGQDRHMIRMRQLIDIVDQLK | | |
| | | R105K, | NYVNDLVPEFLPAPEDVETNCEWSA | | |
| | | P107V | FSCFQDAQLKSANTGNNERIINESI | | |
| | | | KKLKKKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.373662246 | 2778 |
| 8371 | 4177 | D44T, | KSSSQGQDRIMIRMRQLITIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.374863099 | 2779 |
| 8377 | 4177 | D44M, | KSSSQGQDRIMIRMRQLIMIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.377081002 | 2780 |
| 7278 | 4177 | N70G, | KSSSQGQDRIMIRMRQLIDIVDQLK | | |
| | | R105L, | NYVNDLVPEFLPAPEDVETGCEWSA | | |
| | | T110D, | FSCFQKAQLKSANTGNNERIINVSI | | |
| | | A112P | KKLKLKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.380046617 | 2781 |
| 13120 | 6318 | L42K, | KSSSQGQDRFMIRMRQKIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.383847033 | 2782 |
| 17310 | 5845 | K81D, | KSSSQGQDRHMIRMRQLIDIVDQLK | | |
| | | P108A | NYVNDLVPEFLPAPEDVETGCEWSA | | |
| | | | FSCFQDAQLKSANTGNNERIINVSI | | |
| | | | KKLKRKPASTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.393109747 | 2783 |
| 7215 | 4177 | N70G, | KSSSQGQDRIMIRMRQLIDIVDQLK | | |
| | | P108Y, | NYVNDLVPEFLPAPEDVETGCEWSA | | |
| | | T110D, | FSCFQKAQLKSANTGNNERIINVSI | | |
| | | A112P | KKLKRKPYSDNPGRRQKHRLTCPSC | | |
| | | | DSYEKKPPKEFLERFKSLLQKMIHQ | | |
| | | | HLSSRTHGSEDS | | |
| IL-21_ | IL-21_ | P62L, | MRSSPGNMERIVICLMVIFLGTLVH | 2.393606458 | 2784 |
| 16490 | 5845 | N70G, | KSSSQGQDRHMIRMRQLIDIVDQLK | | |
| | | K81D | NYVNDLVPEFLLAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.397737741 | 2785 |
| 16828 | 5845 | K81F | KSSSQGQDRHMIRMRQLIDIVDQLK | | |
| | | | NYVNDLVPEFLPAPEDVETGCEWSA | | |
| | | | FSCFQFAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37F, | MRSSPGNMERIVICLMVIFLGTLVH | 2.402782204 | 2786 |
| 16036 | 5845 | N70G, | KSSSQGQDRHMFRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.405329419 | 2787 |
| 7571 | 4177 | N70G, | KSSSQGQDRIMIRMRQLIDIVDQLK | | |
| | | T89F, | NYVNDLVPEFLPAPEDVETGCEWSA | | |
| | | T110D, | FSCFQKAQLKSANFGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R34G, | MRSSPGNMERIVICLMVIFLGTLVH | 2.408168989 | 2788 |
| 12894 | 0910 | I45T, | KSSSQGQDGHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.409372926 | 2789 |
| 14132 | 6318 | K81D, | KSSSQGQDRFMIRMRQLIDIVDQLK | | |
| | | V98R | NYVNDLVPEFLPAPEDVETNCEWSA | | |
| | | | FSCFQDAQLKSANTGNNERIINRSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.410763539 | 2790 |
| 16826 | 5845 | K81A | KSSSQGQDRHMIRMRQLIDIVDQLK | | |
| | | | NYVNDLVPEFLPAPEDVETGCEWSA | | |
| | | | FSCFQAAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.410794614 | 2791 |
| 8231 | 4177 | Y52N, | KSSSQGQDRIMIRMRQLIDIVDQLK | | |
| | | N70G, | NNVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.417786418 | 2792 |
| 15600 | 6018 | K81D, | KSSSQGQDRFMIRMRQLIDIVDQLK | | |
| | | 196S, | NYVNDLVPEFLPAPEDVETNCEWSA | | |
| | | V98E, | FSCFQDAQLKSANTGNNERISNESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | R38F, | MRSSPGNMERIVICLMVIFLGTLVH | 2.423028688 | 2793 |
| 16054 | 5845 | N70G, | KSSSQGQDRHMIFMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.426454524 | 2794 |
| 16808 | 5845 | Q80A, | KSSSQGQDRHMIRMRQLIDIVDQLK | | |
| | | K81D | NYVNDLVPEFLPAPEDVETGCEWSA | | |
| | | | FSCFADAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44N, | MRSSPGNMERIVICLMVIFLGTLVH | 2.430205336 | 2795 |
| 16168 | 5845 | N70G, | KSSSQGQDRHMIRMRQLINIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.430823477 | 2796 |
| 17392 | 5845 | K81D, | KSSSQGQDRHMIRMRQLIDIVDQLK | | |
| | | A112R | NYVNDLVPEFLPAPEDVETGCEWSA | | |
| | | | FSCFQDAQLKSANTGNNERIINVSI | | |
| | | | KKLKRKPPSTNRGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.436120113 | 2797 |
| 7192 | 4177 | N70G, | KSSSQGQDRIMIRMRQLIDIVDQLK | | |
| | | T110H, | NYVNDLVPEFLPAPEDVETGCEWSA | | |
| | | A112P | FSCFQKAQLKSANTGNNERIINVSI | | |
| | | | KKLKRKPPSHNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.438378799 | 2798 |
| 15659 | 6018 | K81D, | KSSSQGQDRFMIRMRQLIDIVDQLK | | |
| | | V98E, | NYVNDLVPEFLPAPEDVETNCEWSA | | |
| | | I100D, | FSCFQDAQLKSANTGNNERIINESD | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.439139595 | 2799 |
| 17119 | 5845 | K81D, | KSSSQGQDRHMIRMRQLIDIVDQLK | | |
| | | N97H | NYVNDLVPEFLPAPEDVETGCEWSA | | |
| | | | FSCFQDAQLKSANTGNNERIIHVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.446263937 | 2800 |
| 17410 | 5845 | K81D, | KSSSQGQDRHMIRMRQLIDIVDQLK | | |
| | | G113R | NYVNDLVPEFLPAPEDVETGCEWSA | | |
| | | | FSCFQDAQLKSANTGNNERIINVSI | | |
| | | | KKLKRKPPSTNARRRQKHRLTCPSC | | |
| | | | DSYEKKPPKEFLERFKSLLQKMIHQ | | |
| | | | HLSSRTHGSEDS | | |
| IL-21_ | IL-21_ | R34D, | MRSSPGNMERIVICLMVIFLGTLVH | 2.453555415 | 2801 |
| 8562 | 4177 | H35I, | KSSSQGQDDIMIRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R34D, | MRSSPGNMERIVICLMVIFLGTLVH | 2.455208785 | 2802 |
| 15982 | 5845 | N70G, | KSSSQGQDDHMIRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.455399497 | 2803 |
| 17288 | 5845 | K81D, | KSSSQGQDRHMIRMRQLIDIVDQLK | | |
| | | K106S | NYVNDLVPEFLPAPEDVETGCEWSA | | |
| | | | FSCFQDAQLKSANTGNNERIINVSI | | |
| | | | KKLKRSPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70M, | MRSSPGNMERIVICLMVIFLGTLVH | 2.455531491 | 2804 |
| 16634 | 5845 | K81D | KSSSQGQDRHMIRMRQLIDIVDQLK | | |
| | | | NYVNDLVPEFLPAPEDVETMCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.458407793 | 2805 |
| 7269 | 4177 | N70G, | KSSSQGQDRIMIRMRQLIDIVDQLK | | |
| | | R105Y, | NYVNDLVPEFLPAPEDVETGCEWSA | | |
| | | T110D, | FSCFQKAQLKSANTGNNERIINVSI | | |
| | | A112P | KKLKYKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.461225856 | 2806 |
| 7245 | 4177 | N70G, | KSSSQGQDRIMIRMRQLIDIVDQLK | | |
| | | P107H, | NYVNDLVPEFLPAPEDVETGCEWSA | | |
| | | T110D, | FSCFQKAQLKSANTGNNERIINVSI | | |
| | | A112P | KKLKRKHPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.464806401 | 2807 |
| 7551 | 4177 | N70G, | KSSSQGQDRIMIRMRQLIDIVDQLK | | |
| | | G90I, | NYVNDLVPEFLPAPEDVETGCEWSA | | |
| | | T110D, | FSCFQKAQLKSANTINNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.466687841 | 2808 |
| 7504 | 4177 | N70G, | KSSSQGQDRIMIRMRQLIDIVDQLK | | |
| | | N92W, | NYVNDLVPEFLPAPEDVETGCEWSA | | |
| | | T110D, | FSCFQKAQLKSANTGNWERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.468097472 | 2809 |
| 16823 | 5845 | Q80V, | KSSSQGQDRHMIRMRQLIDIVDQLK | | |
| | | K81D | NYVNDLVPEFLPAPEDVETGCEWSA | | |
| | | | FSCFVDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38K, | MRSSPGNMERIVICLMVIFLGTLVH | 2.494937637 | 2810 |
| 19064 | 5474 | N70G, | KSSSQGQDRHMIKMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.498848827 | 2811 |
| 16831 | 5845 | K81I | KSSSQGQDRHMIRMRQLIDIVDQLK | | |
| | | | NYVNDLVPEFLPAPEDVETGCEWSA | | |
| | | | FSCFQIAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.503972333 | 2812 |
| 16837 | 5845 | K81Q | KSSSQGQDRHMIRMRQLIDIVDQLK | | |
| | | | NYVNDLVPEFLPAPEDVETGCEWSA | | |
| | | | FSCFQQAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38G, | MRSSPGNMERIVICLMVIFLGTLVH | 2.508054904 | 2813 |
| 10655 | 3610 | P107V, | KSSSQGQDRHMIGMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | M36P, | MRSSPGNMERIVICLMVIFLGTLVH | 2.511104582 | 2814 |
| 16025 | 5845 | N70G, | KSSSQGQDRHPIRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37P, | MRSSPGNMERIVICLMVIFLGTLVH | 2.514942085 | 2815 |
| 16043 | 5845 | N70G, | KSSSQGQDRHMPRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.522553903 | 2816 |
| 14608 | 6018 | Q41R, | KSSSQGQDRFMIRMRRLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.527628663 | 2817 |
| 7216 | 4177 | N70G, | KSSSQGQDRIMIRMRQLIDIVDQLK | | |
| | | P108W, | NYVNDLVPEFLPAPEDVETGCEWSA | | |
| | | T11OD, | FSCFQKAQLKSANTGNNERIINVSI | | |
| | | A112P | KKLKRKPWSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.536062332 | 2818 |
| 16859 | 5845 | K81D, | KSSSQGQDRHMIRMRQLIDIVDQLK | | |
| | | A82V | NYVNDLVPEFLPAPEDVETGCEWSA | | |
| | | | FSCFQDVQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44W, | MRSSPGNMERIVICLMVIFLGTLVH | 2.547481059 | 2819 |
| 12704 | 0910 | I45T, | KSSSQGQDRHMIRMRQLIWTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.549119856 | 2820 |
| 16712 | 5845 | S74R, | KSSSQGQDRHMIRMRQLIDIVDQLK | | |
| | | K81D | NYVNDLVPEFLPAPEDVETGCEWRA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.551501777 | 2821 |
| 7734 | 4177 | N70G, | KSSSQGQDRIMIRMRQLIDIVDQLK | | |
| | | Q80E, | NYVNDLVPEFLPAPEDVETGCEWSA | | |
| | | T110D, | FSCFEKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.555177259 | 2822 |
| 17314 | 5845 | K81D, | KSSSQGQDRHMIRMRQLIDIVDQLK | | |
| | | P108G | NYVNDLVPEFLPAPEDVETGCEWSA | | |
| | | | FSCFQDAQLKSANTGNNERIINVSI | | |
| | | | KKLKRKPGSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.557491354 | 2823 |
| 7414 | 4177 | N70G, | KSSSQGQDRIMIRMRQLIDIVDQLK | | |
| | | N97W, | NYVNDLVPEFLPAPEDVETGCEWSA | | |
| | | T110D, | FSCFQKAQLKSANTGNNERIIWVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.571758009 | 2824 |
| 7510 | 4177 | N70G, | KSSSQGQDRIMIRMRQLIDIVDQLK | | |
| | | N92P, | NYVNDLVPEFLPAPEDVETGCEWSA | | |
| | | T110D, | FSCFQKAQLKSANTGNPERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | D47G, | MRSSPGNMERIVICLMVIFLGTLVH | 2.572226303 | 2825 |
| 16215 | 5845 | N70G, | KSSSQGQDRHMIRMRQLIDIVGQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.572493085 | 2826 |
| 16675 | 5845 | E72R, | KSSSQGQDRHMIRMRQLIDIVDQLK | | |
| | | K81D | NYVNDLVPEFLPAPEDVETGCRWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.582368969 | 2827 |
| 8378 | 4177 | D44L, | KSSSQGQDRIMIRMRQLILIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.591617882 | 2828 |
| 7727 | 4177 | N70G, | KSSSQGQDRIMIRMRQLIDIVDQLK | | |
| | | Q80M, | NYVNDLVPEFLPAPEDVETGCEWSA | | |
| | | T110D, | FSCFMKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.5980863 | 2829 |
| 16877 | 5845 | K81D, | KSSSQGQDRHMIRMRQLIDIVDQLK | | |
| | | Q83V | NYVNDLVPEFLPAPEDVETGCEWSA | | |
| | | | FSCFQDAVLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.601780463 | 2830 |
| 16833 | 5845 | K81L | KSSSQGQDRHMIRMRQLIDIVDQLK | | |
| | | | NYVNDLVPEFLPAPEDVETGCEWSA | | |
| | | | FSCFQLAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44T, | MRSSPGNMERIVICLMVIFLGTLVH | 2.608516191 | 2831 |
| 16173 | 5845 | N70G, | KSSSQGQDRHMIRMRQLITIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44L, | MRSSPGNMERIVICLMVIFLGTLVH | 2.62310379 | 2832 |
| 16166 | 5845 | N70G, | KSSSQGQDRHMIRMRQLILIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.623302381 | 2833 |
| 17104 | 5845 | K81D, | KSSSQGQDRHMIRMRQLIDIVDQLK | | |
| | | 196M | NYVNDLVPEFLPAPEDVETGCEWSA | | |
| | | | FSCFQDAQLKSANTGNNERIMNVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.635990659 | 2834 |
| 14248 | 6318 | K81D, | KSSSQGQDRFMIRMRQLIDIVDQLK | | |
| | | V98E, | NYVNDLVPEFLPAPEDVETNCEWSA | | |
| | | R105F | FSCFQDAQLKSANTGNNERIINESI | | |
| | | | KKLKFKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.644676649 | 2835 |
| 8511 | 4177 | I37A, | KSSSQGQDRIMARMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.649459276 | 2836 |
| 15753 | 6018 | K81D, | KSSSQGQDRFMIRMRQLIDIVDQLK | | |
| | | V98E, | NYVNDLVPEFLPAPEDVETNCEWSA | | |
| | | R105H, | FSCFQDAQLKSANTGNNERIINESI | | |
| | | T110D | KKLKHKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.653283196 | 2837 |
| 13049 | 6318 | R38L, | KSSSQGQDRFMILMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.656681292 | 2838 |
| 13053 | 6318 | R38Q, | KSSSQGQDRFMIQMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37W, | MRSSPGNMERIVICLMVIFLGTLVH | 2.657747906 | 2839 |
| 16049 | 5845 | N70G, | KSSSQGQDRHMWRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70R, | MRSSPGNMERIVICLMVIFLGTLVH | 2.663688686 | 2840 |
| 16638 | 5845 | K81D | KSSSQGQDRHMIRMRQLIDIVDQLK | | |
| | | | NYVNDLVPEFLPAPEDVETRCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70W, | MRSSPGNMERIVICLMVIFLGTLVH | 2.665361621 | 2841 |
| 16642 | 5845 | K81D | KSSSQGQDRHMIRMRQLIDIVDQLK | | |
| | | | NYVNDLVPEFLPAPEDVETWCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.666337783 | 2842 |
| 7724 | 4177 | N70G, | KSSSQGQDRIMIRMRQLIDIVDQLK | | |
| | | Q80R, | NYVNDLVPEFLPAPEDVETGCEWSA | | |
| | | T110D, | FSCFRKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.668012751 | 2843 |
| 8229 | 4177 | Y52Q, | KSSSQGQDRIMIRMRQLIDIVDQLK | | |
| | | N70G, | NQVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38K, | MRSSPGNMERIVICLMVIFLGTLVH | 2.668792411 | 2844 |
| 10652 | 3610 | P107V, | KSSSQGQDRHMIKMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.67289903 | 2845 |
| 14097 | 6318 | K81D, | KSSSQGQDRFMIRMRQLIDIVDQLK | | |
| | | 196S, | NYVNDLVPEFLPAPEDVETNCEWSA | | |
| | | V98E | FSCFQDAQLKSANTGNNERISNESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.674310461 | 2846 |
| 7728 | 4177 | N70G, | KSSSQGQDRIMIRMRQLIDIVDQLK | | |
| | | Q80L, | NYVNDLVPEFLPAPEDVETGCEWSA | | |
| | | T110D, | FSCFLKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.676341508 | 2847 |
| 7620 | 4177 | N70G, | KSSSQGQDRIMIRMRQLIDIVDQLK | | |
| | | S86L, | NYVNDLVPEFLPAPEDVETGCEWSA | | |
| | | T110D, | FSCFQKAQLKLANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | D44M, | MRSSPGNMERIVICLMVIFLGTLVH | 2.692199857 | 2848 |
| 16167 | 5845 | N70G, | KSSSQGQDRHMIRMRQLIMIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44P, | MRSSPGNMERIVICLMVIFLGTLVH | 2.692737856 | 2849 |
| 17672 | 5707 | K81D, | KSSSQGQDRHMIRMRQLIPIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105K, | MRSSPGNMERIVICLMVIFLGTLVH | 2.701224253 | 2850 |
| 9448 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKKKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.715749053 | 2851 |
| 20118 | 5474 | K81D, | KSSSQGQDRHMIRMRQLIDIVDQLK | | |
| | | 196Y, | NYVNDLVPEFLPAPEDVETGCEWSA | | |
| | | R114Q, | FSCFQDAQLKSANTGNNERIYNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R34W, | MRSSPGNMERIVICLMVIFLGTLVH | 2.718412983 | 2852 |
| 15997 | 5845 | N70G, | KSSSQGQDWHMIRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105M, | MRSSPGNMERIVICLMVIFLGTLVH | 2.721161267 | 2853 |
| 9446 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKMKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45Q, | MRSSPGNMERIVICLMVIFLGTLVH | 2.726334267 | 2854 |
| 16188 | 5845 | N70G, | KSSSQGQDRHMIRMRQLIDQVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 2.726618644 | 2855 |
| 11676 | 0910 | K81D, | KSSSQGQDRHMIRMRQLIDTVDQLK | | |
| | | V98E, | NYVNDLVPEFLPAPEDVETNCEWSA | | |
| | | K102E | FSCFQDAQLKSANTGNNERIINESI | | |
| | | | KELKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.727394872 | 2856 |
| 14684 | 6018 | I45W, | KSSSQGQDRFMIRMRQLIDWVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.729528665 | 2857 |
| 20185 | 5474 | K81D, | KSSSQGQDRHMIRMRQLIDIVDQLK | | |
| | | I100R, | NYVNDLVPEFLPAPEDVETGCEWSA | | |
| | | R114Q, | FSCFQDAQLKSANTGNNERIINVSR | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.731308687 | 2858 |
| 15662 | 6018 | K81D, | KSSSQGQDRFMIRMRQLIDIVDQLK | | |
| | | V98E, | NYVNDLVPEFLPAPEDVETNCEWSA | | |
| | | I100G, | FSCFQDAQLKSANTGNNERIINESG | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.739195456 | 2859 |
| 17110 | 5845 | K81D, | KSSSQGQDRHMIRMRQLIDIVDQLK | | |
| | | 196T | NYVNDLVPEFLPAPEDVETGCEWSA | | |
| | | | FSCFQDAQLKSANTGNNERITNVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.739989913 | 2860 |
| 8376 | 4177 | D44N, | KSSSQGQDRIMIRMRQLINIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38W, | MRSSPGNMERIVICLMVIFLGTLVH | 2.746274627 | 2861 |
| 10643 | 3610 | P107V, | KSSSQGQDRHMIWMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | D44H, | MRSSPGNMERIVICLMVIFLGTLVH | 2.754179299 | 2862 |
| 16163 | 5845 | N70G, | KSSSQGQDRHMIRMRQLIHIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.757141758 | 2863 |
| 14126 | 6318 | K81D, | KSSSQGQDRFMIRMRQLIDIVDQLK | | |
| | | V98K | NYVNDLVPEFLPAPEDVETNCEWSA | | |
| | | | FSCFQDAQLKSANTGNNERIINKSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | L56V, | MRSSPGNMERIVICLMVIFLGTLVH | 2.762131591 | 2864 |
| 16389 | 5845 | N70G, | KSSSQGQDRHMIRMRQLIDIVDQLK | | |
| | | K81D | NYVNDVVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38S, | MRSSPGNMERIVICLMVIFLGTLVH | 2.769738629 | 2865 |
| 19070 | 5474 | N70G, | KSSSQGQDRHMISMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.78539456 | 2866 |
| 17320 | 5845 | K81D, | KSSSQGQDRHMIRMRQLIDIVDQLK | | |
| | | P108N | NYVNDLVPEFLPAPEDVETGCEWSA | | |
| | | | FSCFQDAQLKSANTGNNERIINVSI | | |
| | | | KKLKRKPNSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.789039153 | 2867 |
| 7220 | 4177 | N70G, | KSSSQGQDRIMIRMRQLIDIVDQLK | | |
| | | P108R, | NYVNDLVPEFLPAPEDVETGCEWSA | | |
| | | T110D, | FSCFQKAQLKSANTGNNERIINVSI | | |
| | | A112P | KKLKRKPRSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38N, | MRSSPGNMERIVICLMVIFLGTLVH | 2.798910553 | 2868 |
| 19067 | 5474 | N70G, | KSSSQGQDRHMINMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.8009417 | 2869 |
| 8372 | 4177 | D44S, | KSSSQGQDRIMIRMRQLISIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.8038699 | 2870 |
| 17170 | 5845 | K81D, | KSSSQGQDRHMIRMRQLIDIVDQLK | | |
| | | I100F | NYVNDLVPEFLPAPEDVETGCEWSA | | |
| | | | FSCFQDAQLKSANTGNNERIINVSF | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.819119165 | 2871 |
| 7277 | 4177 | N70G, | KSSSQGQDRIMIRMRQLIDIVDQLK | | |
| | | R105M, | NYVNDLVPEFLPAPEDVETGCEWSA | | |
| | | T11OD, | FSCFQKAQLKSANTGNNERIINVSI | | |
| | | A112P | KKLKMKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.820651658 | 2872 |
| 8301 | 4177 | Q48R, | KSSSQGQDRIMIRMRQLIDIVDRLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.826366965 | 2873 |
| 8227 | 4177 | Y52S, | KSSSQGQDRIMIRMRQLIDIVDQLK | | |
| | | N70G, | NSVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.828170951 | 2874 |
| 15758 | 6018 | K81D, | KSSSQGQDRFMIRMRQLIDIVDQLK | | |
| | | V98E, | NYVNDLVPEFLPAPEDVETNCEWSA | | |
| | | R105N, | FSCFQDAQLKSANTGNNERIINESI | | |
| | | T110D | KKLKNKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.831109699 | 2875 |
| 7661 | 4177 | N70G, | KSSSQGQDRIMIRMRQLIDIVDQLK | | |
| | | L84F, | NYVNDLVPEFLPAPEDVETGCEWSA | | |
| | | T110D, | FSCFQKAQFKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 2.836745154 | 2876 |
| 11581 | 0910 | K81D, | KSSSQGQDRHMIRMRQLIDTVDQLK | | |
| | | V98E, | NYVNDLVPEFLPAPEDVETNCEWSA | | |
| | | P107K | FSCFQDAQLKSANTGNNERIINESI | | |
| | | | KKLKRKKPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.838282098 | 2877 |
| 20243 | 5474 | K81D, | KSSSQGQDRHMIRMRQLIDIVDQLK | | |
| | | L103W, | NYVNDLVPEFLPAPEDVETGCEWSA | | |
| | | R114Q, | FSCFQDAQLKSANTGNNERIINVSI | | |
| | | R115D | KKWKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.84906597 | 2878 |
| 8226 | 4177 | Y52T, | KSSSQGQDRIMIRMRQLIDIVDQLK | | |
| | | N70G, | NTVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.854741602 | 2879 |
| 16843 | 5845 | K81Y | KSSSQGQDRHMIRMRQLIDIVDQLK | | |
| | | | NYVNDLVPEFLPAPEDVETGCEWSA | | |
| | | | FSCFQYAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.877542296 | 2880 |
| 8381 | 4177 | D44H, | KSSSQGQDRIMIRMRQLIHIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.900902787 | 2881 |
| 20272 | 5474 | K81D, | KSSSQGQDRHMIRMRQLIDIVDQLK | | |
| | | R105M, | NYVNDLVPEFLPAPEDVETGCEWSA | | |
| | | R114Q, | FSCFQDAQLKSANTGNNERIINVSI | | |
| | | R115D | KKLKMKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.906096052 | 2882 |
| 16679 | 5845 | E72W, | KSSSQGQDRHMIRMRQLIDIVDQLK | | |
| | | K81D | NYVNDLVPEFLPAPEDVETGCWWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.910241775 | 2883 |
| 13125 | 6318 | L42R, | KSSSQGQDRFMIRMRQRIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38L, | MRSSPGNMERIVICLMVIFLGTLVH | 2.915413978 | 2884 |
| 19065 | 5474 | N70G, | KSSSQGQDRHMILMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.921081443 | 2885 |
| 8484 | 4177 | R38M, | KSSSQGQDRIMIMMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.932868824 | 2886 |
| 20190 | 5474 | K81D, | KSSSQGQDRHMIRMRQLIDIVDQLK | | |
| | | I100Y, | NYVNDLVPEFLPAPEDVETGCEWSA | | |
| | | R114Q, | FSCFQDAQLKSANTGNNERIINVSY | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37P, | MRSSPGNMERIVICLMVIFLGTLVH | 2.946588817 | 2887 |
| 12836 | 0910 | I45T, | KSSSQGQDRHMPRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 2.947975805 | 2888 |
| 20184 | 5474 | K81D, | KSSSQGQDRHMIRMRQLIDIVDQLK | | |
| | | I100Q, | NYVNDLVPEFLPAPEDVETGCEWSA | | |
| | | R114Q, | FSCFQDAQLKSANTGNNERIINVSQ | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G | MRSSPGNMERIVICLMVIFLGTLVH | 2.96193522 | 2889 |
| 16832 | 5845 | | KSSSQGQDRHMIRMRQLIDIVDQLK | | |
| | | | NYVNDLVPEFLPAPEDVETGCEWSA | | |
| | | | FSCFQKAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.965522302 | 2890 |
| 7048 | 4177 | N70G, | KSSSQGQDRIMIRMRQLIDIVDQLK | | |
| | | T110D, | NYVNDLVPEFLPAPEDVETGCEWSA | | |
| | | A112P, | FSCFQKAQLKSANTGNNERIINVSI | | |
| | | H118K | KKLKRKPPSDNPGRRQKKRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.988154844 | 2891 |
| 7238 | 4177 | N70G, | KSSSQGQDRIMIRMRQLIDIVDQLK | | |
| | | P107R, | NYVNDLVPEFLPAPEDVETGCEWSA | | |
| | | T110D, | FSCFQKAQLKSANTGNNERIINVSI | | |
| | | A112P | KKLKRKRPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 2.988623386 | 2892 |
| 8506 | 4177 | I37H, | KSSSQGQDRIMHRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 2.996417155 | 2893 |
| 15592 | 6018 | K81D, | KSSSQGQDRFMIRMRQLIDIVDQLK | | |
| | | 196H, | NYVNDLVPEFLPAPEDVETNCEWSA | | |
| | | V98E, | FSCFQDAQLKSANTGNNERIHNESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | R38Q, | MRSSPGNMERIVICLMVIFLGTLVH | 3.036568151 | 2894 |
| 10647 | 3610 | P107V, | KSSSQGQDRHMIQMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.051342816 | 2895 |
| 15233 | 6018 | F76K, | KSSSQGQDRFMIRMRQLIDIVDQLK | | |
| | | K81D, | NYVNDLVPEFLPAPEDVETNCEWSA | | |
| | | V98E, | KSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.11364762 | 2896 |
| 7423 | 4177 | N70G, | KSSSQGQDRIMIRMRQLIDIVDQLK | | |
| | | N97K, | NYVNDLVPEFLPAPEDVETGCEWSA | | |
| | | T110D, | FSCFQKAQLKSANTGNNERIIKVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.119360137 | 2897 |
| 8502 | 4177 | I37N, | KSSSQGQDRIMNRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.127169028 | 2898 |
| 16820 | 5845 | Q80R, | KSSSQGQDRHMIRMRQLIDIVDQLK | | |
| | | K81D | NYVNDLVPEFLPAPEDVETGCEWSA | | |
| | | | FSCFRDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.13173756 | 2899 |
| 17090 | 5845 | K81D, | KSSSQGQDRHMIRMRQLIDIVDQLK | | |
| | | 195R | NYVNDLVPEFLPAPEDVETGCEWSA | | |
| | | | FSCFQDAQLKSANTGNNERRINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.136367258 | 2900 |
| 8507 | 4177 | I37G, | KSSSQGQDRIMGRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 3.138455943 | 2901 |
| 18639 | 5707 | V98K, | KSSSQGQDRHMIRMRQLIDIVDQLK | | |
| | | P107V | NYVNDLVPEFLPAPEDVETNCEWSA | | |
| | | | FSCFQDAQLKSANTGNNERIINKSI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44F, | MRSSPGNMERIVICLMVIFLGTLVH | 3.142408176 | 2902 |
| 16161 | 5845 | N70G, | KSSSQGQDRHMIRMRQLIFIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | D44Y, | MRSSPGNMERIVICLMVIFLGTLVH | 3.151484246 | 2903 |
| 16176 | 5845 | N70G, | KSSSQGQDRHMIRMRQLIYIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37D, | MRSSPGNMERIVICLMVIFLGTLVH | 3.153238055 | 2904 |
| 12845 | 0910 | I45T, | KSSSQGQDRHMDRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.160704939 | 2905 |
| 7405 | 4177 | N70G, | KSSSQGQDRIMIRMRQLIDIVDQLK | | |
| | | V98K, | NYVNDLVPEFLPAPEDVETGCEWSA | | |
| | | T110D, | FSCFQKAQLKSANTGNNERIINKSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.164223781 | 2906 |
| 13106 | 6318 | Q41P, | KSSSQGQDRFMIRMRPLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.166775952 | 2907 |
| 16830 | 5845 | K81H | KSSSQGQDRHMIRMRQLIDIVDQLK | | |
| | | | NYVNDLVPEFLPAPEDVETGCEWSA | | |
| | | | FSCFQHAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38M, | MRSSPGNMERIVICLMVIFLGTLVH | 3.191390571 | 2908 |
| 16060 | 5845 | N70G, | KSSSQGQDRHMIMMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.199335005 | 2909 |
| 7283 | 4177 | N70G, | KSSSQGQDRIMIRMRQLIDIVDQLK | | |
| | | R105F, | NYVNDLVPEFLPAPEDVETGCEWSA | | |
| | | T110D, | FSCFQKAQLKSANTGNNERIINVSI | | |
| | | A112P | KKLKFKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.199692791 | 2910 |
| 14255 | 6318 | K81D, | KSSSQGQDRFMIRMRQLIDIVDQLK | | |
| | | V98E, | NYVNDLVPEFLPAPEDVETNCEWSA | | |
| | | R105N | FSCFQDAQLKSANTGNNERIINESI | | |
| | | | KKLKNKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.230208648 | 2911 |
| 7490 | 4177 | N70G, | KSSSQGQDRIMIRMRQLIDIVDQLK | | |
| | | E93R, | NYVNDLVPEFLPAPEDVETGCEWSA | | |
| | | T110D, | FSCFQKAQLKSANTGNNRRIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45W, | MRSSPGNMERIVICLMVIFLGTLVH | 3.231264694 | 2912 |
| 16192 | 5845 | N70G, | KSSSQGQDRHMIRMRQLIDWVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.243193738 | 2913 |
| 7615 | 4177 | N70G, | KSSSQGQDRIMIRMRQLIDIVDQLK | | |
| | | S86R, | NYVNDLVPEFLPAPEDVETGCEWSA | | |
| | | T110D, | FSCFQKAQLKRANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38V, | MRSSPGNMERIVICLMVIFLGTLVH | 3.244615323 | 2914 |
| 10644 | 3610 | P107V, | KSSSQGQDRHMIVMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.277082194 | 2915 |
| 16825 | 5845 | Q80Y, | KSSSQGQDRHMIRMRQLIDIVDQLK | | |
| | | K81D | NYVNDLVPEFLPAPEDVETGCEWSA | | |
| | | | FSCFYDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.281300673 | 2916 |
| 7500 | 4177 | N70G, | KSSSQGQDRIMIRMRQLIDIVDQLK | | |
| | | E93F, | NYVNDLVPEFLPAPEDVETGCEWSA | | |
| | | T110D, | FSCFQKAQLKSANTGNNFRIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.286103083 | 2917 |
| 8499 | 4177 | I37R, | KSSSQGQDRIMRRMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.28782226 | 2918 |
| 8356 | 4177 | I45Q, | KSSSQGQDRIMIRMRQLIDQVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I37G, | MRSSPGNMERIVICLMVIFLGTLVH | 3.302128872 | 2919 |
| 16037 | 5845 | N70G, | KSSSQGQDRHMGRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.309233762 | 2920 |
| 7373 | 4177 | N70G, | KSSSQGQDRIMIRMRQLIDIVDQLK | | |
| | | I100F, | NYVNDLVPEFLPAPEDVETGCEWSA | | |
| | | T110D, | FSCFQKAQLKSANTGNNERIINVSF | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.315420414 | 2921 |
| 16809 | 5845 | Q80D, | KSSSQGQDRHMIRMRQLIDIVDQLK | | |
| | | K81D | NYVNDLVPEFLPAPEDVETGCEWSA | | |
| | | | FSCFDDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Q41W, | MRSSPGNMERIVICLMVIFLGTLVH | 3.327691602 | 2922 |
| 12758 | 0910 | I45T, | KSSSQGQDRHMIRMRWLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I37R, | MRSSPGNMERIVICLMVIFLGTLVH | 3.351454304 | 2923 |
| 16045 | 5845 | N70G, | KSSSQGQDRHMRRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.368156674 | 2924 |
| 15754 | 6018 | K81D, | KSSSQGQDRFMIRMRQLIDIVDQLK | | |
| | | V98E, | NYVNDLVPEFLPAPEDVETNCEWSA | | |
| | | R105I, | FSCFQDAQLKSANTGNNERIINESI | | |
| | | T110D | KKLKIKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | R105W, | MRSSPGNMERIVICLMVIFLGTLVH | 3.371181863 | 2925 |
| 9439 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKWKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.375843198 | 2926 |
| 8486 | 4177 | R38K, | KSSSQGQDRIMIKMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.381621332 | 2927 |
| 7720 | 4177 | N70G, | KSSSQGQDRIMIRMRQLIDIVDQLK | | |
| | | Q80W, | NYVNDLVPEFLPAPEDVETGCEWSA | | |
| | | T110D, | FSCFWKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.389250362 | 2928 |
| 17180 | 5845 | K81D, | KSSSQGQDRHMIRMRQLIDIVDQLK | | |
| | | I100S | NYVNDLVPEFLPAPEDVETGCEWSA | | |
| | | | FSCFQDAQLKSANTGNNERIINVSS | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.399062081 | 2929 |
| 7225 | 4177 | N70G, | KSSSQGQDRIMIRMRQLIDIVDQLK | | |
| | | P108K, | NYVNDLVPEFLPAPEDVETGCEWSA | | |
| | | T110D, | FSCFQKAQLKSANTGNNERIINVSI | | |
| | | A112P | KKLKRKPKSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.400389857 | 2930 |
| 8368 | 4177 | D44Y, | KSSSQGQDRIMIRMRQLIYIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.404819539 | 2931 |
| 8383 | 4177 | D44F, | KSSSQGQDRIMIRMRQLIFIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.411539655 | 2932 |
| 16741 | 5845 | F76K, | KSSSQGQDRHMIRMRQLIDIVDQLK | | |
| | | K81D | NYVNDLVPEFLPAPEDVETGCEWSA | | |
| | | | KSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38W, | MRSSPGNMERIVICLMVIFLGTLVH | 3.411894421 | 2933 |
| 12812 | 0910 | I45T, | KSSSQGQDRHMIWMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38S, | MRSSPGNMERIVICLMVIFLGTLVH | 3.41405973 | 2934 |
| 10646 | 3610 | P107V, | KSSSQGQDRHMISMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.418452293 | 2935 |
| 19801 | 5474 | F79I, | KSSSQGQDRHMIRMRQLIDIVDQLK | | |
| | | K81D, | NYVNDLVPEFLPAPEDVETGCEWSA | | |
| | | R114Q, | FSCIQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | M39G, | MRSSPGNMERIVICLMVIFLGTLVH | 3.434670766 | 2936 |
| 12806 | 0910 | I45T, | KSSSQGQDRHMIRGRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.44986491 | 2937 |
| 20175 | 5474 | K81D, | KSSSQGQDRHMIRMRQLIDIVDQLK | | |
| | | I100E, | NYVNDLVPEFLPAPEDVETGCEWSA | | |
| | | R114Q, | FSCFQDAQLKSANTGNNERIINVSE | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R105Q, | MRSSPGNMERIVICLMVIFLGTLVH | 3.455455431 | 2938 |
| 9443 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKQKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.459453592 | 2939 |
| 7286 | 4177 | N70G, | KSSSQGQDRIMIRMRQLIDIVDQLK | | |
| | | R105A, | NYVNDLVPEFLPAPEDVETGCEWSA | | |
| | | T110D, | FSCFQKAQLKSANTGNNERIINVSI | | |
| | | A112P | KKLKAKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.461896853 | 2940 |
| 7276 | 4177 | N70G, | KSSSQGQDRIMIRMRQLIDIVDQLK | | |
| | | R105N, | NYVNDLVPEFLPAPEDVETGCEWSA | | |
| | | T110D, | FSCFQKAQLKSANTGNNERIINVSI | | |
| | | A112P | KKLKNKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 3.47204279 | 2941 |
| 18770 | 5707 | V98E, | KSSSQGQDRHMIRMRQLIDIVDQLK | | |
| | | R105Q, | NYVNDLVPEFLPAPEDVETNCEWSA | | |
| | | P107V | FSCFQDAQLKSANTGNNERIINESI | | |
| | | | KKLKQKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.478147566 | 2942 |
| 15748 | 6018 | K81D, | KSSSQGQDRFMIRMRQLIDIVDQLK | | |
| | | V98E, | NYVNDLVPEFLPAPEDVETNCEWSA | | |
| | | R105A, | FSCFQDAQLKSANTGNNERIINESI | | |
| | | T110D | KKLKAKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 3.487864361 | 2943 |
| 18771 | 5707 | V98E, | KSSSQGQDRHMIRMRQLIDIVDQLK | | |
| | | R105S, | NYVNDLVPEFLPAPEDVETNCEWSA | | |
| | | P107V | FSCFQDAQLKSANTGNNERIINESI | | |
| | | | KKLKSKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.496746132 | 2944 |
| 17054 | 5845 | K81D, | KSSSQGQDRHMIRMRQLIDIVDQLK | | |
| | | E93R | NYVNDLVPEFLPAPEDVETGCEWSA | | |
| | | | FSCFQDAQLKSANTGNNRRIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.506972125 | 2945 |
| 7387 | 4177 | N70G, | KSSSQGQDRIMIRMRQLIDIVDQLK | | |
| | | S99K, | NYVNDLVPEFLPAPEDVETGCEWSA | | |
| | | T110D, | FSCFQKAQLKSANTGNNERIINVKI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I37K, | MRSSPGNMERIVICLMVIFLGTLVH | 3.514697591 | 2946 |
| 12840 | 0910 | I45T, | KSSSQGQDRHMKRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105S, | MRSSPGNMERIVICLMVIFLGTLVH | 3.520588841 | 2947 |
| 9442 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKSKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.523301371 | 2948 |
| 8155 | 4177 | L56T, | KSSSQGQDRIMIRMRQLIDIVDQLK | | |
| | | N70G, | NYVNDTVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.524841311 | 2949 |
| 20105 | 5474 | K81D, | KSSSQGQDRHMIRMRQLIDIVDQLK | | |
| | | 196G, | NYVNDLVPEFLPAPEDVETGCEWSA | | |
| | | R114Q, | FSCFQDAQLKSANTGNNERIGNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 3.530023215 | 2950 |
| 18764 | 5707 | V98E, | KSSSQGQDRHMIRMRQLIDIVDQLK | | |
| | | R105I, | NYVNDLVPEFLPAPEDVETNCEWSA | | |
| | | P107V | FSCFQDAQLKSANTGNNERIINESI | | |
| | | | KKLKIKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38T, | MRSSPGNMERIVICLMVIFLGTLVH | 3.549898707 | 2951 |
| 17568 | 5707 | K81D, | KSSSQGQDRHMITMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38V, | MRSSPGNMERIVICLMVIFLGTLVH | 3.568737131 | 2952 |
| 17569 | 5707 | K81D, | KSSSQGQDRHMIVMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.569542306 | 2953 |
| 8228 | 4177 | Y52R, | KSSSQGQDRIMIRMRQLIDIVDQLK | | |
| | | N70G, | NRVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.569702787 | 2954 |
| 17138 | 5845 | K81D, | KSSSQGQDRHMIRMRQLIDIVDQLK | | |
| | | V98K | NYVNDLVPEFLPAPEDVETGCEWSA | | |
| | | | FSCFQDAQLKSANTGNNERIINKSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.575916274 | 2955 |
| 7679 | 4177 | N70G, | KSSSQGQDRIMIRMRQLIDIVDQLK | | |
| | | Q83F, | NYVNDLVPEFLPAPEDVETGCEWSA | | |
| | | T110D, | FSCFQKAFLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I37N, | MRSSPGNMERIVICLMVIFLGTLVH | 3.578238601 | 2956 |
| 16042 | 5845 | N70G, | KSSSQGQDRHMNRMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.581433031 | 2957 |
| 16815 | 5845 | Q80K, | KSSSQGQDRHMIRMRQLIDIVDQLK | | |
| | | K81D | NYVNDLVPEFLPAPEDVETGCEWSA | | |
| | | | FSCFKDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.584276883 | 2958 |
| 8481 | 4177 | R38Q, | KSSSQGQDRIMIQMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.585853039 | 2959 |
| 7274 | 4177 | N70G, | KSSSQGQDRIMIRMRQLIDIVDQLK | | |
| | | R105Q, | NYVNDLVPEFLPAPEDVETGCEWSA | | |
| | | T110D, | FSCFQKAQLKSANTGNNERIINVSI | | |
| | | A112P | KKLKQKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.597961607 | 2960 |
| 16865 | 5845 | K81D, | KSSSQGQDRHMIRMRQLIDIVDQLK | | |
| | | Q83F | NYVNDLVPEFLPAPEDVETGCEWSA | | |
| | | | FSCFQDAFLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.615626647 | 2961 |
| 8373 | 4177 | D44R, | KSSSQGQDRIMIRMRQLIRIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.620616267 | 2962 |
| 20102 | 5474 | K81D, | KSSSQGQDRHMIRMRQLIDIVDQLK | | |
| | | 196D, | NYVNDLVPEFLPAPEDVETGCEWSA | | |
| | | R114Q, | FSCFQDAQLKSANTGNNERIDNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.621109833 | 2963 |
| 20111 | 5474 | K81D, | KSSSQGQDRHMIRMRQLIDIVDQLK | | |
| | | 196P, | NYVNDLVPEFLPAPEDVETGCEWSA | | |
| | | R114Q, | FSCFQDAQLKSANTGNNERIPNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.623816296 | 2964 |
| 20177 | 5474 | K81D, | KSSSQGQDRHMIRMRQLIDIVDQLK | | |
| | | I100G, | NYVNDLVPEFLPAPEDVETGCEWSA | | |
| | | R114Q, | FSCFQDAQLKSANTGNNERIINVSG | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.623851026 | 2965 |
| 16870 | 5845 | K81D, | KSSSQGQDRHMIRMRQLIDIVDQLK | | |
| | | Q83L | NYVNDLVPEFLPAPEDVETGCEWSA | | |
| | | | FSCFQDALLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.628637807 | 2966 |
| 20244 | 5474 | K81D, | KSSSQGQDRHMIRMRQLIDIVDQLK | | |
| | | L103Y, | NYVNDLVPEFLPAPEDVETGCEWSA | | |
| | | R114Q, | FSCFQDAQLKSANTGNNERIINVSI | | |
| | | R115D | KKYKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.630497738 | 2967 |
| 20280 | 5474 | K81D, | KSSSQGQDRHMIRMRQLIDIVDQLK | | |
| | | R105Y, | NYVNDLVPEFLPAPEDVETGCEWSA | | |
| | | R114Q, | FSCFQDAQLKSANTGNNERIINVSI | | |
| | | R115D | KKLKYKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R105N, | MRSSPGNMERIVICLMVIFLGTLVH | 3.63934268 | 2968 |
| 9445 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKNKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I37E, | MRSSPGNMERIVICLMVIFLGTLVH | 3.640175379 | 2969 |
| 12844 | 0910 | I45T, | KSSSQGQDRHMERMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.643252905 | 2970 |
| 7434 | 4177 | N70G, | KSSSQGQDRIMIRMRQLIDIVDQLK | | |
| | | 196T, | NYVNDLVPEFLPAPEDVETGCEWSA | | |
| | | T110D, | FSCFQKAQLKSANTGNNERITNVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.645383827 | 2971 |
| 17224 | 5845 | K81D, | KSSSQGQDRHMIRMRQLIDIVDQLK | | |
| | | L103F | NYVNDLVPEFLPAPEDVETGCEWSA | | |
| | | | FSCFQDAQLKSANTGNNERIINVSI | | |
| | | | KKEKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.646303864 | 2972 |
| 14556 | 6018 | R38T, | KSSSQGQDRFMITMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.648609508 | 2973 |
| 7271 | 4177 | N70G, | KSSSQGQDRIMIRMRQLIDIVDQLK | | |
| | | R105V, | NYVNDLVPEFLPAPEDVETGCEWSA | | |
| | | T110D, | FSCFQKAQLKSANTGNNERIINVSI | | |
| | | A112P | KKLKVKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.653548636 | 2974 |
| 8493 | 4177 | R38A, | KSSSQGQDRIMIAMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.654421624 | 2975 |
| 7280 | 4177 | N70G, | KSSSQGQDRIMIRMRQLIDIVDQLK | | |
| | | R105I, | NYVNDLVPEFLPAPEDVETGCEWSA | | |
| | | T110D, | FSCFQKAQLKSANTGNNERIINVSI | | |
| | | A112P | KKLKIKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38I, | MRSSPGNMERIVICLMVIFLGTLVH | 3.663092481 | 2976 |
| 17560 | 5707 | K81D, | KSSSQGQDRHMIIMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.66817399 | 2977 |
| 14674 | 6018 | I45K, | KSSSQGQDRFMIRMRQLIDKVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 3.674727658 | 2978 |
| 11617 | 0910 | K81D, | KSSSQGQDRHMIRMRQLIDTVDQLK | | |
| | | V98E, | NYVNDLVPEFLPAPEDVETNCEWSA | | |
| | | R105K | FSCFQDAQLKSANTGNNERIINESI | | |
| | | | KKLKKKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.684875474 | 2979 |
| 15760 | 6018 | K81D, | KSSSQGQDRFMIRMRQLIDIVDQLK | | |
| | | V98E, | NYVNDLVPEFLPAPEDVETNCEWSA | | |
| | | R105Q, | FSCFQDAQLKSANTGNNERIINESI | | |
| | | T110D | KKLKQKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.687663366 | 2980 |
| 17160 | 5845 | K81D, | KSSSQGQDRHMIRMRQLIDIVDQLK | | |
| | | S99P | NYVNDLVPEFLPAPEDVETGCEWSA | | |
| | | | FSCFQDAQLKSANTGNNERIINVPI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.690168988 | 2981 |
| 13051 | 6318 | R38N, | KSSSQGQDRFMINMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.698970737 | 2982 |
| 20114 | 5474 | K81D, | KSSSQGQDRHMIRMRQLIDIVDQLK | | |
| | | 196S, | NYVNDLVPEFLPAPEDVETGCEWSA | | |
| | | R114Q, | FSCFQDAQLKSANTGNNERISNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.707179585 | 2983 |
| 20275 | 5474 | K81D, | KSSSQGQDRHMIRMRQLIDIVDQLK | | |
| | | R105Q, | NYVNDLVPEFLPAPEDVETGCEWSA | | |
| | | R114Q, | FSCFQDAQLKSANTGNNERIINVSI | | |
| | | R115D | KKLKQKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.708291658 | 2984 |
| 7272 | 4177 | N70G, | KSSSQGQDRIMIRMRQLIDIVDQLK | | |
| | | R105T, | NYVNDLVPEFLPAPEDVETGCEWSA | | |
| | | T110D, | FSCFQKAQLKSANTGNNERIINVSI | | |
| | | A112P | KKLKTKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | Q41P, | MRSSPGNMERIVICLMVIFLGTLVH | 3.710581777 | 2985 |
| 19122 | 5474 | N70G, | KSSSQGQDRHMIRMRPLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.7121124 | 2986 |
| 20266 | 5474 | K81D, | KSSSQGQDRHMIRMRQLIDIVDQLK | | |
| | | R105F, | NYVNDLVPEFLPAPEDVETGCEWSA | | |
| | | R114Q, | FSCFQDAQLKSANTGNNERIINVSI | | |
| | | R115D | KKLKFKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | Q41K, | MRSSPGNMERIVICLMVIFLGTLVH | 3.725656386 | 2987 |
| 19118 | 5474 | N70G, | KSSSQGQDRHMIRMRKLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.739763992 | 2988 |
| 14261 | 6318 | K81D, | KSSSQGQDRFMIRMRQLIDIVDQLK | | |
| | | V98E, | NYVNDLVPEFLPAPEDVETNCEWSA | | |
| | | R105W | FSCFQDAQLKSANTGNNERIINESI | | |
| | | | KKLKWKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.740015673 | 2989 |
| 15761 | 6018 | K81D, | KSSSQGQDRFMIRMRQLIDIVDQLK | | |
| | | V98E, | NYVNDLVPEFLPAPEDVETNCEWSA | | |
| | | R105S, | FSCFQDAQLKSANTGNNERIINESI | | |
| | | T110D | KKLKSKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.744825187 | 2990 |
| 8379 | 4177 | D44K, | KSSSQGQDRIMIRMRQLIKIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R34E, | MRSSPGNMERIVICLMVIFLGTLVH | 3.777914781 | 2991 |
| 12896 | 0910 | I45T, | KSSSQGQDEHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.77984391 | 2992 |
| 17162 | 5845 | K81D, | KSSSQGQDRHMIRMRQLIDIVDQLK | | |
| | | S99R | NYVNDLVPEFLPAPEDVETGCEWSA | | |
| | | | FSCFQDAQLKSANTGNNERIINVRI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.791582231 | 2993 |
| 7666 | 4177 | N70G, | KSSSQGQDRIMIRMRQLIDIVDQLK | | |
| | | Q83W, | NYVNDLVPEFLPAPEDVETGCEWSA | | |
| | | T110D, | FSCFQKAWLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.80013393 | 2994 |
| 15762 | 6018 | K81D, | KSSSQGQDRFMIRMRQLIDIVDQLK | | |
| | | V98E, | NYVNDLVPEFLPAPEDVETNCEWSA | | |
| | | R105T, | FSCFQDAQLKSANTGNNERIINESI | | |
| | | T110D | KKLKTKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.801152304 | 2995 |
| 13047 | 6318 | R38I, | KSSSQGQDREMIIMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38T, | MRSSPGNMERIVICLMVIFLGTLVH | 3.806207894 | 2996 |
| 16065 | 5845 | N70G, | KSSSQGQDRHMITMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105I, | MRSSPGNMERIVICLMVIFLGTLVH | 3.808276608 | 2997 |
| 9449 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKIKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38I, | MRSSPGNMERIVICLMVIFLGTLVH | 3.808961926 | 2998 |
| 16057 | 5845 | N70G, | KSSSQGQDRHMIIMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.822920385 | 2999 |
| 8479 | 4177 | R38T, | KSSSQGQDRIMITMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.830604898 | 3000 |
| 13042 | 6318 | R38D, | KSSSQGQDRFMIDMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Q41K, | MRSSPGNMERIVICLMVIFLGTLVH | 3.841211608 | 3001 |
| 16112 | 5845 | N70G, | KSSSQGQDRHMIRMRKLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.849028724 | 3002 |
| 13730 | 6318 | F76K, | KSSSQGQDRFMIRMRQLIDIVDQLK | | |
| | | K81D, | NYVNDLVPEFLPAPEDVETNCEWSA | | |
| | | V98E | KSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.855516361 | 3003 |
| 14245 | 6318 | K81D, | KSSSQGQDRFMIRMRQLIDIVDQLK | | |
| | | V98E, | NYVNDLVPEFLPAPEDVETNCEWSA | | |
| | | R105A | FSCFQDAQLKSANTGNNERIINESI | | |
| | | | KKLKAKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38L, | MRSSPGNMERIVICLMVIFLGTLVH | 3.8561938 | 3004 |
| 17562 | 5707 | K81D, | KSSSQGQDRHMILMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38N, | MRSSPGNMERIVICLMVIFLGTLVH | 3.856925011 | 3005 |
| 10649 | 3610 | P107V, | KSSSQGQDRHMINMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.858793268 | 3006 |
| 15764 | 6018 | K81D, | KSSSQGQDRFMIRMRQLIDIVDQLK | | |
| | | V98E, | NYVNDLVPEFLPAPEDVETNCEWSA | | |
| | | R105W, | FSCFQDAQLKSANTGNNERIINESI | | |
| | | T110D | KKLKWKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | R38A, | MRSSPGNMERIVICLMVIFLGTLVH | 3.876216314 | 3007 |
| 16051 | 5845 | N70GK81D | KSSSQGQDRHMIAMRQLIDIVDQLK | | |
| | | | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.879852172 | 3008 |
| 17265 | 5845 | K81D, | KSSSQGQDRHMIRMRQLIDIVDQLK | | |
| | | R105L | NYVNDLVPEFLPAPEDVETGCEWSA | | |
| | | | FSCFQDAQLKSANTGNNERIINVSI | | |
| | | | KKLKLKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.888941657 | 3009 |
| 17322 | 5845 | K81D, | KSSSQGQDRHMIRMRQLIDIVDQLK | | |
| | | P108R | NYVNDLVPEFLPAPEDVETGCEWSA | | |
| | | | FSCFQDAQLKSANTGNNERIINVSI | | |
| | | | KKLKRKPRSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38V, | MRSSPGNMERIVICLMVIFLGTLVH | 3.896952246 | 3010 |
| 19072 | 5474 | N70G, | KSSSQGQDRHMIVMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38N, | MRSSPGNMERIVICLMVIFLGTLVH | 3.90112088 | 3011 |
| 17564 | 5707 | K81D, | KSSSQGQDRHMINMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38I, | MRSSPGNMERIVICLMVIFLGTLVH | 3.901230692 | 3012 |
| 12822 | 0910 | I45T, | KSSSQGQDRHMIIMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38D, | MRSSPGNMERIVICLMVIFLGTLVH | 3.909666172 | 3013 |
| 17555 | 5707 | K81D, | KSSSQGQDRHMIDMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 3.925613904 | 3014 |
| 11622 | 0910 | K81D, | KSSSQGQDRHMIRMRQLIDTVDQLK | | |
| | | V98E, | NYVNDLVPEFLPAPEDVETNCEWSA | | |
| | | R105E | FSCFQDAQLKSANTGNNERIINESI | | |
| | | | KKLKEKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.932332122 | 3015 |
| 17274 | 5845 | K81D, | KSSSQGQDRHMIRMRQLIDIVDQLK | | |
| | | R105Y | NYVNDLVPEFLPAPEDVETGCEWSA | | |
| | | | FSCFQDAQLKSANTGNNERIINVSI | | |
| | | | KKLKYKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.93763486 | 3016 |
| 17236 | 5845 | K81D, | KSSSQGQDRHMIRMRQLIDIVDQLK | | |
| | | L103V | NYVNDLVPEFLPAPEDVETGCEWSA | | |
| | | | FSCFQDAQLKSANTGNNERIINVSI | | |
| | | | KKVKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 3.945929914 | 3017 |
| 18762 | 5707 | V98E, | KSSSQGQDRHMIRMRQLIDIVDQLK | | |
| | | R105G, | NYVNDLVPEFLPAPEDVETNCEWSA | | |
| | | P107V | FSCFQDAQLKSANTGNNERIINESI | | |
| | | | KKLKGKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38L, | MRSSPGNMERIVICLMVIFLGTLVH | 3.946176705 | 3018 |
| 12820 | 0910 | I45T, | KSSSQGQDRHMILMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38D, | MRSSPGNMERIVICLMVIFLGTLVH | 3.950887324 | 3019 |
| 12827 | 0910 | I45T, | KSSSQGQDRHMIDMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 3.963326266 | 3020 |
| 15663 | 6018 | K81D, | KSSSQGQDRFMIRMRQLIDIVDQLK | | |
| | | V98E, | NYVNDLVPEFLPAPEDVETNCEWSA | | |
| | | I100H, | FSCFQDAQLKSANTGNNERIINESH | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 3.963455059 | 3021 |
| 7307 | 4177 | N70G, | KSSSQGQDRIMIRMRQLIDIVDQLK | | |
| | | L103V, | NYVNDLVPEFLPAPEDVETGCEWSA | | |
| | | T110D, | FSCFQKAQLKSANTGNNERIINVSI | | |
| | | A112P | KKVKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.966205226 | 3022 |
| 16878 | 5845 | K81D, | KSSSQGQDRHMIRMRQLIDIVDQLK | | |
| | | Q83W | NYVNDLVPEFLPAPEDVETGCEWSA | | |
| | | | FSCFQDAWLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R34D, | MRSSPGNMERIVICLMVIFLGTLVH | 3.970170078 | 3023 |
| 12897 | 0910 | I45T, | KSSSQGQDDHMIRMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.98455441 | 3024 |
| 20273 | 5474 | K81D, | KSSSQGQDRHMIRMRQLIDIVDQLK | | |
| | | R105N, | NYVNDLVPEFLPAPEDVETGCEWSA | | |
| | | R114Q, | FSCFQDAQLKSANTGNNERIINVSI | | |
| | | R115D | KKLKNKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38N, | MRSSPGNMERIVICLMVIFLGTLVH | 3.984608757 | 3025 |
| 12818 | 0910 | I45T, | KSSSQGQDRHMINMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 3.992191659 | 3026 |
| 17266 | 5845 | K81DR105M | KSSSQGQDRHMIRMRQLIDIVDQLK | | |
| | | | NYVNDLVPEFLPAPEDVETGCEWSA | | |
| | | | FSCFQDAQLKSANTGNNERIINVSI | | |
| | | | KKLKMKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.01136832 | 3027 |
| 8351 | 4177 | I45W, | KSSSQGQDRIMIRMRQLIDWVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.016841295 | 3028 |
| 17098 | 5845 | K81D, | KSSSQGQDRHMIRMRQLIDIVDQLK | | |
| | | 196E | NYVNDLVPEFLPAPEDVETGCEWSA | | |
| | | | FSCFQDAQLKSANTGNNERIENVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.017409114 | 3029 |
| 20174 | 5474 | K81D, | KSSSQGQDRHMIRMRQLIDIVDQLK | | |
| | | I100D, | NYVNDLVPEFLPAPEDVETGCEWSA | | |
| | | R114Q, | FSCFQDAQLKSANTGNNERIINVSD | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38P, | MRSSPGNMERIVICLMVIFLGTLVH | 4.027278482 | 3030 |
| 10648 | 3610 | P107V, | KSSSQGQDRHMIPMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38D, | MRSSPGNMERIVICLMVIFLGTLVH | 4.027943568 | 3031 |
| 19058 | 5474 | N70G, | KSSSQGQDRHMIDMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.031778951 | 3032 |
| 8492 | 4177 | R38D, | KSSSQGQDRIMIDMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.032392008 | 3033 |
| 11616 | 0910 | K81D, | KSSSQGQDRHMIRMRQLIDTVDQLK | | |
| | | V98E, | NYVNDLVPEFLPAPEDVETNCEWSA | | |
| | | R105L | FSCFQDAQLKSANTGNNERIINESI | | |
| | | | KKLKLKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.036728475 | 3034 |
| 17105 | 5845 | K81D, | KSSSQGQDRHMIRMRQLIDIVDQLK | | |
| | | 196N | NYVNDLVPEFLPAPEDVETGCEWSA | | |
| | | | FSCFQDAQLKSANTGNNERINNVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.044170164 | 3035 |
| 17259 | 5845 | K81D, | KSSSQGQDRHMIRMRQLIDIVDQLK | | |
| | | R105E | NYVNDLVPEFLPAPEDVETGCEWSA | | |
| | | | FSCFQDAQLKSANTGNNERIINVSI | | |
| | | | KKLKEKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.057089258 | 3036 |
| 8427 | 4177 | Q41R, | KSSSQGQDRIMIRMRRLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38E, | MRSSPGNMERIVICLMVIFLGTLVH | 4.057254479 | 3037 |
| 12826 | 0910 | I45T, | KSSSQGQDRHMIEMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 4.058120175 | 3038 |
| 14543 | 6018 | R38D, | KSSSQGQDRFMIDMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | R38E, | MRSSPGNMERIVICLMVIFLGTLVH | 4.064375956 | 3039 |
| 10657 | 3610 | P107V, | KSSSQGQDRHMIEMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.065786238 | 3040 |
| 8480 | 4177 | R38S, | KSSSQGQDRIMISMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.066663741 | 3041 |
| 7284 | 4177 | N70G, | KSSSQGQDRIMIRMRQLIDIVDQLK | | |
| | | R105E, | NYVNDLVPEFLPAPEDVETGCEWSA | | |
| | | T110D, | FSCFQKAQLKSANTGNNERIINVSI | | |
| | | A112P | KKLKEKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.070021509 | 3042 |
| 20178 | 5474 | K81D, | KSSSQGQDRHMIRMRQLIDIVDQLK | | |
| | | I100H, | NYVNDLVPEFLPAPEDVETGCEWSA | | |
| | | R114Q, | FSCFQDAQLKSANTGNNERIINVSH | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.072042578 | 3043 |
| 20113 | 5474 | K81D, | KSSSQGQDRHMIRMRQLIDIVDQLK | | |
| | | 196R, | NYVNDLVPEFLPAPEDVETGCEWSA | | |
| | | R114Q, | FSCFQDAQLKSANTGNNERIRNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45K, | MRSSPGNMERIVICLMVIFLGTLVH | 4.073006502 | 3044 |
| 16183 | 5845 | N70G, | KSSSQGQDRHMIRMRQLIDKVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.074023537 | 3045 |
| 17257 | 5845 | K81D, | KSSSQGQDRHMIRMRQLIDIVDQLK | | |
| | | R105A | NYVNDLVPEFLPAPEDVETGCEWSA | | |
| | | | FSCFQDAQLKSANTGNNERIINVSI | | |
| | | | KKLKAKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.075129027 | 3046 |
| 8485 | 4177 | R38L, | KSSSQGQDRIMILMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.075411528 | 3047 |
| 11610 | 0910 | K81D, | KSSSQGQDRHMIRMRQLIDTVDQLK | | |
| | | V98E, | NYVNDLVPEFLPAPEDVETNCEWSA | | |
| | | R105T | FSCFQDAQLKSANTGNNERIINESI | | |
| | | | KKLKTKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.079244581 | 3048 |
| 7281 | 4177 | N70G, | KSSSQGQDRIMIRMRQLIDIVDQLK | | |
| | | R105H, | NYVNDLVPEFLPAPEDVETGCEWSA | | |
| | | T110D, | FSCFQKAQLKSANTGNNERIINVSI | | |
| | | A112P | KKLKHKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38S, | MRSSPGNMERIVICLMVIFLGTLVH | 4.101457912 | 3049 |
| 16064 | 5845 | N70G, | KSSSQGQDRHMISMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.114072725 | 3050 |
| 17263 | 5845 | K81D, | KSSSQGQDRHMIRMRQLIDIVDQLK | | |
| | | R105I | NYVNDLVPEFLPAPEDVETGCEWSA | | |
| | | | FSCFQDAQLKSANTGNNERIINVSI | | |
| | | | KKLKIKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38S, | MRSSPGNMERIVICLMVIFLGTLVH | 4.114600169 | 3051 |
| 12815 | 0910 | I45T, | KSSSQGQDRHMISMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.115980247 | 3052 |
| 11611 | 0910 | K81D, | KSSSQGQDRHMIRMRQLIDTVDQLK | | |
| | | V98E, | NYVNDLVPEFLPAPEDVETNCEWSA | | |
| | | R105S | FSCFQDAQLKSANTGNNERIINESI | | |
| | | | KKLKSKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38L, | MRSSPGNMERIVICLMVIFLGTLVH | 4.125978963 | 3053 |
| 10651 | 3610 | P107V, | KSSSQGQDRHMILMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 4.129523722 | 3054 |
| 13056 | 6318 | R38V, | KSSSQGQDRFMIVMRQLIDIVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 4.133518559 | 3055 |
| 14260 | 6318 | K81D, | KSSSQGQDRFMIRMRQLIDIVDQLK | | |
| | | V98E, | NYVNDLVPEFLPAPEDVETNCEWSA | | |
| | | R105V | FSCFQDAQLKSANTGNNERIINESI | | |
| | | | KKLKVKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.13362881 | 3056 |
| 7448 | 4177 | N70G, | KSSSQGQDRIMIRMRQLIDIVDQLK | | |
| | | 196A, | NYVNDLVPEFLPAPEDVETGCEWSA | | |
| | | T110D, | FSCFQKAQLKSANTGNNERIANVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38P, | MRSSPGNMERIVICLMVIFLGTLVH | 4.135393327 | 3057 |
| 12817 | 0910 | I45T, | KSSSQGQDRHMIPMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.138645716 | 3058 |
| 11607 | 0910 | K81D, | KSSSQGQDRHMIRMRQLIDTVDQLK | | |
| | | V98E, | NYVNDLVPEFLPAPEDVETNCEWSA | | |
| | | R105Y | FSCFQDAQLKSANTGNNERIINESI | | |
| | | | KKLKYKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 4.14296174 | 3059 |
| 13173 | 6318 | I45K, | KSSSQGQDRFMIRMRQLIDKVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105V, | MRSSPGNMERIVICLMVIFLGTLVH | 4.14736135 | 3060 |
| 9440 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKVKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.154299485 | 3061 |
| 19740 | 5474 | A75Y, | KSSSQGQDRHMIRMRQLIDIVDQLK | | |
| | | K81D, | NYVNDLVPEFLPAPEDVETGCEWSY | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.155447488 | 3062 |
| 20229 | 5474 | K81D, | KSSSQGQDRHMIRMRQLIDIVDQLK | | |
| | | L103E, | NYVNDLVPEFLPAPEDVETGCEWSA | | |
| | | R114Q, | FSCFQDAQLKSANTGNNERIINVSI | | |
| | | R115D | KKEKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.165015394 | 3063 |
| 20265 | 5474 | K81D, | KSSSQGQDRHMIRMRQLIDIVDQLK | | |
| | | R105E, | NYVNDLVPEFLPAPEDVETGCEWSA | | |
| | | R114Q, | FSCFQDAQLKSANTGNNERIINVSI | | |
| | | R115D | KKLKEKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.168505012 | 3064 |
| 20238 | 5474 | K81D, | KSSSQGQDRHMIRMRQLIDIVDQLK | | |
| | | L103Q, | NYVNDLVPEFLPAPEDVETGCEWSA | | |
| | | R114Q, | FSCFQDAQLKSANTGNNERIINVSI | | |
| | | R115D | KKQKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | Q41K, | MRSSPGNMERIVICLMVIFLGTLVH | 4.177367825 | 3065 |
| 12767 | 0910 | I45T, | KSSSQGQDRHMIRMRKLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H351, | MRSSPGNMERIVICLMVIFLGTLVH | 4.177384607 | 3066 |
| 8355 | 4177 | I45R, | KSSSQGQDRIMIRMRQLIDRVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 4.177614169 | 3067 |
| 13179 | 6318 | I45R, | KSSSQGQDRFMIRMRQLIDRVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.190114758 | 3068 |
| 17264 | 5845 | K81D, | KSSSQGQDRHMIRMRQLIDIVDQLK | | |
| | | R105K | NYVNDLVPEFLPAPEDVETGCEWSA | | |
| | | | FSCFQDAQLKSANTGNNERIINVSI | | |
| | | | KKLKKKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Q41R, | MRSSPGNMERIVICLMVIFLGTLVH | 4.190188292 | 3069 |
| 16117 | 5845 | N70G, | KSSSQGQDRHMIRMRRLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R105G, | MRSSPGNMERIVICLMVIFLGTLVH | 4.191527254 | 3070 |
| 9451 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKGKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.198670002 | 3071 |
| 17258 | 5845 | K81D, | KSSSQGQDRHMIRMRQLIDIVDQLK | | |
| | | R105D | NYVNDLVPEFLPAPEDVETGCEWSA | | |
| | | | FSCFQDAQLKSANTGNNERIINVSI | | |
| | | | KKLKDKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.200338495 | 3072 |
| 11608 | 0910 | K81D, | KSSSQGQDRHMIRMRQLIDTVDQLK | | |
| | | V98E, | NYVNDLVPEFLPAPEDVETNCEWSA | | |
| | | R105W | FSCFQDAQLKSANTGNNERIINESI | | |
| | | | KKLKWKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.200566734 | 3073 |
| 17107 | 5845 | K81D, | KSSSQGQDRHMIRMRQLIDIVDQLK | | |
| | | 196Q | NYVNDLVPEFLPAPEDVETGCEWSA | | |
| | | | FSCFQDAQLKSANTGNNERIQNVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.203547217 | 3074 |
| 20232 | 5474 | K81D, | KSSSQGQDRHMIRMRQLIDIVDQLK | | |
| | | L103H, | NYVNDLVPEFLPAPEDVETGCEWSA | | |
| | | R114Q, | FSCFQDAQLKSANTGNNERIINVSI | | |
| | | R115D | KKHKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.204363543 | 3075 |
| 11620 | 0910 | K81D, | KSSSQGQDRHMIRMRQLIDTVDQLK | | |
| | | V98E, | NYVNDLVPEFLPAPEDVETNCEWSA | | |
| | | R105G | FSCFQDAQLKSANTGNNERIINESI | | |
| | | | KKLKGKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.204639537 | 3076 |
| 20228 | 5474 | K81D, | KSSSQGQDRHMIRMRQLIDIVDQLK | | |
| | | L103D, | NYVNDLVPEFLPAPEDVETGCEWSA | | |
| | | R114Q, | FSCFQDAQLKSANTGNNERIINVSI | | |
| | | R115D | KKDKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R38Q, | MRSSPGNMERIVICLMVIFLGTLVH | 4.207688247 | 3077 |
| 16063 | 5845 | N70G, | KSSSQGQDRHMIQMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38P, | MRSSPGNMERIVICLMVIFLGTLVH | 4.209429217 | 3078 |
| 17565 | 5707 | K81D, | KSSSQGQDRHMIPMRQLIDIVDQLK | | |
| | | V98E, | NYVNDLVPEFLPAPEDVETNCEWSA | | |
| | | P107V | FSCFQDAQLKSANTGNNERIINESI | | |
| | | | KKLKRKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.211025302 | 3079 |
| 17223 | 5845 | K81D, | KSSSQGQDRHMIRMRQLIDIVDQLK | | |
| | | L103E | NYVNDLVPEFLPAPEDVETGCEWSA | | |
| | | | FSCFQDAQLKSANTGNNERIINVSI | | |
| | | | KKEKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.215451309 | 3080 |
| 11618 | 0910 | K81D, | KSSSQGQDRHMIRMRQLIDTVDQLK | | |
| | | V98E, | NYVNDLVPEFLPAPEDVETNCEWSA | | |
| | | R105I | FSCFQDAQLKSANTGNNERIINESI | | |
| | | | KKLKIKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38N, | MRSSPGNMERIVICLMVIFLGTLVH | 4.22983306 | 3081 |
| 16061 | 5845 | N70G, | KSSSQGQDRHMINMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.231177227 | 3082 |
| 8489 | 4177 | R38G, | KSSSQGQDRIMIGMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.23182307 | 3083 |
| 11623 | 0910 | K81D, | KSSSQGQDRHMIRMRQLIDTVDQLK | | |
| | | V98E, | NYVNDLVPEFLPAPEDVETNCEWSA | | |
| | | R105D | FSCFQDAQLKSANTGNNERIINESI | | |
| | | | KKLKDKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.23411416 | 3084 |
| 17238 | 5845 | K81D, | KSSSQGQDRHMIRMRQLIDIVDQLK | | |
| | | L103Y | NYVNDLVPEFLPAPEDVETGCEWSA | | |
| | | | FSCFQDAQLKSANTGNNERIINVSI | | |
| | | | KKYKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.241752224 | 3085 |
| 20279 | 5474 | K81D, | KSSSQGQDRHMIRMRQLIDIVDQLK | | |
| | | R105W, | NYVNDLVPEFLPAPEDVETGCEWSA | | |
| | | R114Q, | FSCFQDAQLKSANTGNNERIINVSI | | |
| | | R115D | KKLKWKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.245169565 | 3086 |
| 17267 | 5845 | K81D, | KSSSQGQDRHMIRMRQLIDIVDQLK | | |
| | | R105N | NYVNDLVPEFLPAPEDVETGCEWSA | | |
| | | | FSCFQDAQLKSANTGNNERIINVSI | | |
| | | | KKLKNKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38V, | MRSSPGNMERIVICLMVIFLGTLVH | 4.252938002 | 3087 |
| 16066 | 5845 | N70G, | KSSSQGQDRHMIVMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | Q41W, | MRSSPGNMERIVICLMVIFLGTLVH | 4.254191642 | 3088 |
| 16121 | 5845 | N70G, | KSSSQGQDRHMIRMRWLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.265503244 | 3089 |
| 19865 | 5474 | K81D, | KSSSQGQDRHMIRMRQLIDIVDQLK | | |
| | | A82W, | NYVNDLVPEFLPAPEDVETGCEWSA | | |
| | | R114Q, | FSCFQDWQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.268598014 | 3090 |
| 17234 | 5845 | K81D, | KSSSQGQDRHMIRMRQLIDIVDQLK | | |
| | | L103S | NYVNDLVPEFLPAPEDVETGCEWSA | | |
| | | | FSCFQDAQLKSANTGNNERIINVSI | | |
| | | | KKSKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.270428278 | 3091 |
| 20236 | 5474 | K81D, | KSSSQGQDRHMIRMRQLIDIVDQLK | | |
| | | L103N, | NYVNDLVPEFLPAPEDVETGCEWSA | | |
| | | R114Q, | FSCFQDAQLKSANTGNNERIINVSI | | |
| | | R115D | KKNKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.270808621 | 3092 |
| 17269 | 5845 | K81D, | KSSSQGQDRHMIRMRQLIDIVDQLK | | |
| | | R105Q | NYVNDLVPEFLPAPEDVETGCEWSA | | |
| | | | FSCFQDAQLKSANTGNNERIINVSI | | |
| | | | KKLKQKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.277723283 | 3093 |
| 11613 | 0910 | K81D, | KSSSQGQDRHMIRMRQLIDTVDQLK | | |
| | | V98E, | NYVNDLVPEFLPAPEDVETNCEWSA | | |
| | | R105P | FSCFQDAQLKSANTGNNERIINESI | | |
| | | | KKLKPKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 4.28093546 | 3094 |
| 15749 | 6018 | K81D, | KSSSQGQDRFMIRMRQLIDIVDQLK | | |
| | | V98E, | NYVNDLVPEFLPAPEDVETNCEWSA | | |
| | | R105D, | FSCFQDAQLKSANTGNNERIINESI | | |
| | | T110D | KKLKDKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | R38L, | MRSSPGNMERIVICLMVIFLGTLVH | 4.285445774 | 3095 |
| 16059 | 5845 | N70G, | KSSSQGQDRHMILMRQLIDIVDQLK | | |
| | | K81D | NYVNDLVPEFLPAPEDVETGCEWSA | | |
| | | | FSCFQDAQLKSANTGNNERIINVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38P, | MRSSPGNMERIVICLMVIFLGTLVH | 4.297027381 | 3096 |
| 19068 | 5474 | N70G, | KSSSQGQDRHMIPMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.299377658 | 3097 |
| 17273 | 5845 | K81D, | KSSSQGQDRHMIRMRQLIDIVDQLK | | |
| | | R105W | NYVNDLVPEFLPAPEDVETGCEWSA | | |
| | | | FSCFQDAQLKSANTGNNERIINVSI | | |
| | | | KKLKWKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38M, | MRSSPGNMERIVICLMVIFLGTLVH | 4.299696029 | 3098 |
| 12819 | 0910 | I45T, | KSSSQGQDRHMIMMRQLIDTVDQLK | | |
| | | K81D, | NYVNDLVPEFLPAPEDVETNCEWSA | | |
| | | V98E | FSCFQDAQLKSANTGNNERIINESI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.333413125 | 3099 |
| 7285 | 4177 | N70G, | KSSSQGQDRIMIRMRQLIDIVDQLK | | |
| | | R105D, | NYVNDLVPEFLPAPEDVETGCEWSA | | |
| | | T110D, | FSCFQKAQLKSANTGNNERIINVSI | | |
| | | A112P | KKLKDKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.343984569 | 3100 |
| 11619 | 0910 | K81D, | KSSSQGQDRHMIRMRQLIDTVDQLK | | |
| | | V98E, | NYVNDLVPEFLPAPEDVETNCEWSA | | |
| | | R105H | FSCFQDAQLKSANTGNNERIINESI | | |
| | | | KKLKHKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.347945466 | 3101 |
| 17272 | 5845 | K81D, | KSSSQGQDRHMIRMRQLIDIVDQLK | | |
| | | R105V | NYVNDLVPEFLPAPEDVETGCEWSA | | |
| | | | FSCFQDAQLKSANTGNNERIINVSI | | |
| | | | KKLKVKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | K81D, | MRSSPGNMERIVICLMVIFLGTLVH | 4.349901067 | 3102 |
| 18769 | 5707 | V98E, | KSSSQGQDRHMIRMRQLIDIVDQLK | | |
| | | R105P, | NYVNDLVPEFLPAPEDVETNCEWSA | | |
| | | P107V | FSCFQDAQLKSANTGNNERIINESI | | |
| | | | KKLKPKVPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.357966217 | 3103 |
| 20277 | 5474 | K81D, | KSSSQGQDRHMIRMRQLIDIVDQLK | | |
| | | R105T, | NYVNDLVPEFLPAPEDVETGCEWSA | | |
| | | R114Q, | FSCFQDAQLKSANTGNNERIINVSI | | |
| | | R115D | KKLKTKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | R105D, | MRSSPGNMERIVICLMVIFLGTLVH | 4.369748312 | 3104 |
| 9454 | 3610 | P107V, | KSSSQGQDRHMIRMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKDKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | H35F, | MRSSPGNMERIVICLMVIFLGTLVH | 4.374510525 | 3105 |
| 14544 | 6018 | R38E, | KSSSQGQDRFMIEMRQLIDIVDQLK | | |
| | | K81D, | NYVNDLVPEFLPAPEDVETNCEWSA | | |
| | | V98E, | FSCFQDAQLKSANTGNNERIINESI | | |
| | | T110D | KKLKRKPPSDNAGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.382828635 | 3106 |
| 17262 | 5845 | K81D, | KSSSQGQDRHMIRMRQLIDIVDQLK | | |
| | | R105H | NYVNDLVPEFLPAPEDVETGCEWSA | | |
| | | | FSCFQDAQLKSANTGNNERIINVSI | | |
| | | | KKLKHKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.402751804 | 3107 |
| 7447 | 4177 | N70G, | KSSSQGQDRIMIRMRQLIDIVDQLK | | |
| | | 196D, | NYVNDLVPEFLPAPEDVETGCEWSA | | |
| | | T110D, | FSCFQKAQLKSANTGNNERIDNVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.440190316 | 3108 |
| 17270 | 5845 | K81D, | KSSSQGQDRHMIRMRQLIDIVDQLK | | |
| | | R105S | NYVNDLVPEFLPAPEDVETGCEWSA | | |
| | | | FSCFQDAQLKSANTGNNERIINVSI | | |
| | | | KKLKSKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.447511588 | 3109 |
| 7674 | 4177 | N70G, | KSSSQGQDRIMIRMRQLIDIVDQLK | | |
| | | Q83L, | NYVNDLVPEFLPAPEDVETGCEWSA | | |
| | | T110D, | FSCFQKALLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.453942703 | 3110 |
| 7270 | 4177 | N70G, | KSSSQGQDRIMIRMRQLIDIVDQLK | | |
| | | R105W, | NYVNDLVPEFLPAPEDVETGCEWSA | | |
| | | T11OD, | FSCFQKAQLKSANTGNNERIINVSI | | |
| | | A112P | KKLKWKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.465446324 | 3111 |
| 8487 | 4177 | R38I, | KSSSQGQDRIMIIMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.500922798 | 3112 |
| 20107 | 5474 | K81D, | KSSSQGQDRHMIRMRQLIDIVDQLK | | |
| | | 196K, | NYVNDLVPEFLPAPEDVETGCEWSA | | |
| | | R114Q, | FSCFQDAQLKSANTGNNERIKNVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | I45T, | MRSSPGNMERIVICLMVIFLGTLVH | 4.510978155 | 3113 |
| 11609 | 0910 | K81D, | KSSSQGQDRHMIRMRQLIDTVDQLK | | |
| | | V98E, | NYVNDLVPEFLPAPEDVETNCEWSA | | |
| | | R105V | FSCFQDAQLKSANTGNNERIINESI | | |
| | | | KKLKVKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | H35I, | MRSSPGNMERIVICLMVIFLGTLVH | 4.530671298 | 3114 |
| 8482 | 4177 | R38P, | KSSSQGQDRIMIPMRQLIDIVDQLK | | |
| | | N70G, | NYVNDLVPEFLPAPEDVETGCEWSA | | |
| | | T110D, | FSCFQKAQLKSANTGNNERIINVSI | | |
| | | A112P | KKLKRKPPSDNPGRRQKHRLTCPSC | | |
| | | | DSYEKKPPKEFLERFKSLLQKMIHQ | | |
| | | | HLSSRTHGSEDS | | |
| IL-21_ | IL-21_ | R38I, | MRSSPGNMERIVICLMVIFLGTLVH | 4.534231146 | 3115 |
| 10653 | 3610 | P107V, | KSSSQGQDRHMIIMRQLIDIVDQLK | | |
| | | R114Q, | NYVNDLVPEFLPAPEDVETNCEWSA | | |
| | | R115D | FSCFQKAQLKSANTGNNERIINVSI | | |
| | | | KKLKRKVPSTNAGQDQKHRLTCPSC | | |
| | | | DSYEKKPPKEFLERFKSLLQKMIHQ | | |
| | | | HLSSRTHGSEDS | | |
| IL-21_ | IL-21_ | N70G, | MRSSPGNMERIVICLMVIFLGTLVH | 4.56820794 | 3116 |
| 17101 | 5845 | K81D, | KSSSQGQDRHMIRMRQLIDIVDQLK | | |
| | | 196H | NYVNDLVPEFLPAPEDVETGCEWSA | | |
| | | | FSCFQDAQLKSANTGNNERIHNVSI | | |
| | | | KKLKRKPPSTNAGRRQKHRLTCPSC | | |
| | | | DSYEKKPPKEFLERFKSLLQKMIHQ | | |
| | | | HLSSRTHGSEDS | | |
| IL-21_ | IL-21_ | R38E, | MRSSPGNMERIVICLMVIFLGTLVH | 5.072531317 | 3117 |
| 19059 | 5474 | N70G, | KSSSQGQDRHMIEMRQLIDIVDQLK | | |
| | | K81D, | NYVNDLVPEFLPAPEDVETGCEWSA | | |
| | | R114Q, | FSCFQDAQLKSANTGNNERIINVSI | | |
| | | R115D | KKLKRKPPSTNAGQDQKHRLTCPSC | | |
| | | | DSYEKKPPKEFLERFKSLLQKMIHQ | | |
| | | | HLSSRTHGSEDS | | |

### Example 6: Efficacy Modeling for IL-21v-Anti-CD8 Fusions

To demonstrate the therapeutic effect of delivering detuned, stabilized IL-21 molecules to cytotoxic T cells, a surrogate detuned mouse IL-21 molecule fused to an anti-mouse-CD8 antibody was tested in a syngeneic mouse tumor growth inhibition study. MC38 colon adenocarcinoma cells were subcutaneously implanted in the flank of C57BL/6 mice. Implanted tumors were grown to approximate mean tumor volumes of 80 mm³, as determined by caliper measurement and calculated as L×W×H/2. Non-outlier, tumor bearing mice were randomized into treatment groups consisting of 10 mice per group on DO, and immunocytokine or vehicle test agents were dosed intravenously. Body weights and tumor volume measurements were performed three times a week throughout the duration of the study. Mice received 5 mg/kg of test agent or vehicle control on DO, D7, and D14. Mice were taken off study when tumor volumes exceeded 2000 mm³, lost more than 20% body weight or tumors became ulcerated. In some instances, mice were found dead from tumor or toxicity complications.

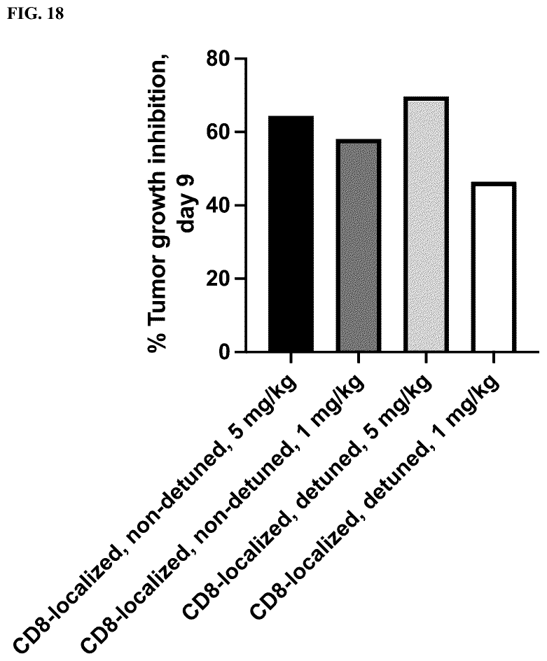

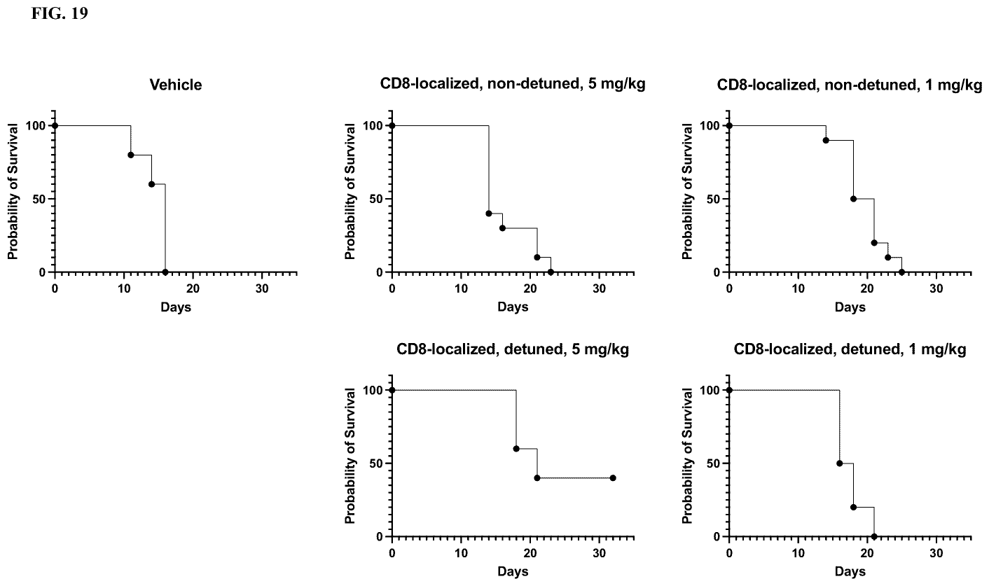

On study day 9, the first mouse in the vehicle treatment group was taken off study and tumor growth inhibition was calculated as (1−(mean tumor volume treatment group/mean tumor volume control group))*100. As shown in FIG. 18, the anti-CD8 localized detuned IL-21 showed similar tumor growth inhibition as anti-CD8 localized stabilized, non-detuned IL-21 cytokine. However, mice treated with the anti-CD8 targeted non-detuned cytokine experienced lethal toxicity in the 24 hours following the third dose (D14), shown in the survival curves in FIG. 19. In contrast, none of the mice treated with anti-CD8 targeted detuned IL-21 exhibited toxicity or needed to be taken off study at that time. These results demonstrate detuned CD8-localized IL-21 elicited increased tumor growth inhibition with a wider therapeutic index compared to the similarly formatted and localized version of the non-detuned IL-21 cytokine.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A human interleukin 21 (IL-21) variant, wherein the IL-21 variant has:
(i) decreased or no binding to a human interleukin 21 receptor (IL-21R) as compared to a wild-type human IL-21 polypeptide; or
(ii) increased stability as compared to a wild-type human IL-21 polypeptide; or
(iii) decreased or no binding to a human interleukin 21 receptor (IL-21R) as compared to a wild-type human IL-21 polypeptide and increased stability as compared to a wild-type human IL-21 polypeptide.
